(19) United States
(12) Patent Application Publication
(10) Pub. No.: US 2005/0075375 A1
(43) Pub. Date: Apr. 7, 2005

(54) Heterocyclic compounds for treating hepatitis C virus

(75) Inventors: Dionisios Vourloumis, San Diego, CA (US); Masayuki Takahashi, San Diego, CA (US); Geoffrey C. Winters, Coquitlam (CA); Jinglan Zhou, San Diego, CA (US); Russell Duchene, San Diego, CA (US)

Correspondence Address: CONNOLLY BOVE LODGE & HUTZ LLP, SUITE 800, 1990 M STREET NW, WASHINGTON, DC, 20036-3425, US

(73) Assignee: Anadys Pharmaceuticals, Inc., San Diego, CA (US)

(21) Appl. No.: 10/845,587

(22) Filed: May 14, 2004

Related U.S. Application Data
(60) Provisional application No. 60/470,200, filed on May 14, 2003.

(51) Int. Cl. A61K 31/535; A61K 31/433; A61K 31/426; A61K 31/421; A61K 31/4178

(52) U.S. Cl. 514/362; 514/364; 514/365; 514/374; 514/396

(57) ABSTRACT

The invention is directed to heterocyclic compounds and pharmaceutical compositions of the same for treating Hepatitis C virus.

This application claims priority to Provisional Application No. 60/470,200 filed May 14, 2003.

The invention is directed to heterocyclic compounds and pharmaceutical compositions thereof that are particularly useful in treating infections by Hepatitis C virus.

Hepatitis C is a major health problem world-wide. The World Health Organization estimates that 170 million people are chronic carriers of the hepatitis C virus (HCV), with 4 million carriers in the United States alone. In the United States, HCV infection accounts for 40% of chronic liver disease and HCV disease is the most common cause for liver transplantation. HCV infection leads to a chronic infection and about 70% of persons infected will develop chronic histological changes in the liver (chronic hepatitis) with a 10-40% risk of cirrhosis and an estimated 4% lifetime risk of hepatocellular carcinoma. The CDC estimates that each year in the United States there are 35,000 new cases of HCV infection and approximately ten thousand deaths attributed to HCV disease.

The current standard of care is a pegylated interferon/ribavirin combination at a cost of approximately $31,000/year. These drugs have difficult dosing problems and side-effects that preclude their use in almost half of diagnosed patients. Pegylated interferon treatment is associated with menacing flu-like symptoms, irritability, inability to concentrate, suicidal ideation, and leukocytopenia. Ribavirin is associated with hemolytic anemia and birth defects.

The overall response to this standard therapy is low; approximately one third of patients do not respond. Of those who do respond, a large fraction relapse within six months of completing 6-12 months of therapy. As a consequence, the long-term response rate for all patients entering treatment is only about 50%. The relatively low response rate and the significant side-effects of current therapy anti-HCV drug treatments, coupled with the negative long term effects of chronic HCV infection, result in a continuing medical need for improved therapy. Antiviral pharmaceuticals to treat RNA virus diseases like HCV are few, and as described above are often associated with multiple adverse effects. While there are, in some cases, medicines available to reduce disease symptoms, there are few drugs to effectively inhibit replication of the underlying virus. The significance and prevalence of RNA virus diseases, including but not limited to chronic infection by the hepatitis C virus, and coupled with the limited availability and effectiveness of current antiviral pharmaceuticals, have created a compelling and continuing need for new pharmaceuticals to treat these diseases.

### SUMMARY OF THE INVENTION

The present invention has addressed this need by the discovery of heterocyclic compounds and pharmaceutically acceptable prodrugs, pharmaceutically active metabolites, and pharmaceutically acceptable salts thereof (such compounds, prodrugs, metabolites and salts are collectively referred to as “agents”) described below, which are useful in the treatment of HCV replication.

In a general aspect, the invention relates to compounds of Formula I: 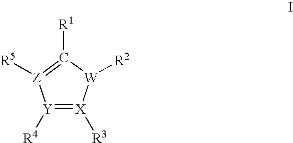
wherein:

X, Y, and Z are independently selected from C and N atoms;

W is selected from N, O, and S atoms;

R¹, R³, R⁴, and R⁵ are independently selected from hydrogen, halogen, nitro, or a unsubstituted or substituted alkyl, alkoxy, aryl, heteroaryl, Ring A, and Ring B, or R⁴ and R⁵ combine to form, together with Y and Z, a five- or six-membered heterocycloalkyl;

R² is selected from hydrogen or an unsubstituted or substituted alkyl, Ring A, and Ring B when W is N;

wherein at least two of W, X, Y, and Z are heteroatoms;

wherein one of the R¹-R⁵ groups is Ring A and one of the R¹-R⁵ groups are Ring B, and the remaining R¹-R⁵ groups are selected from hydrogen, halogen, nitro, or a unsubstituted or substituted alkyl and alkoxy; and

wherein Ring A and Ring B are independently selected from an unsubstituted or substituted aryl, alkylaryl, heteroaryl, alkylheteroaryl, or heterocycloalkyl.

In a specific embodiment, the invention relates to compounds of Formula I selected from

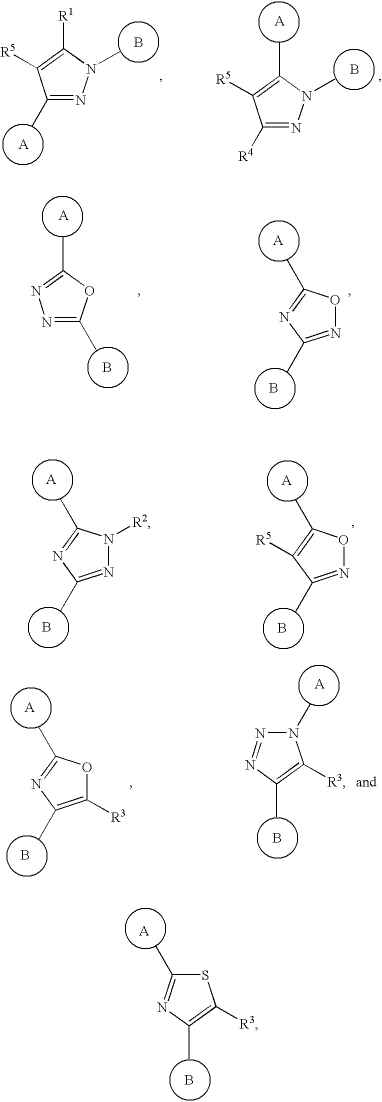

wherein R¹, R², R³, R⁴, and R⁵ are independently selected from hydrogen, halogen, or nitro, or an unsubstituted or substituted alkyl or alkoxy, and Ring A is an unsubstituted or substituted aryl or heteroaryl selected from the group consisting of 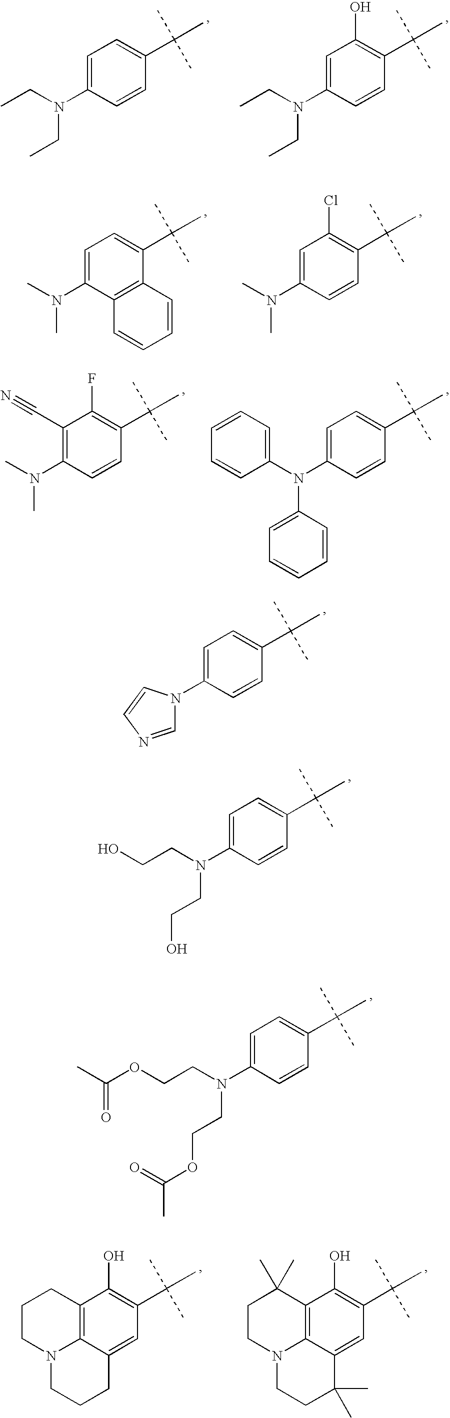 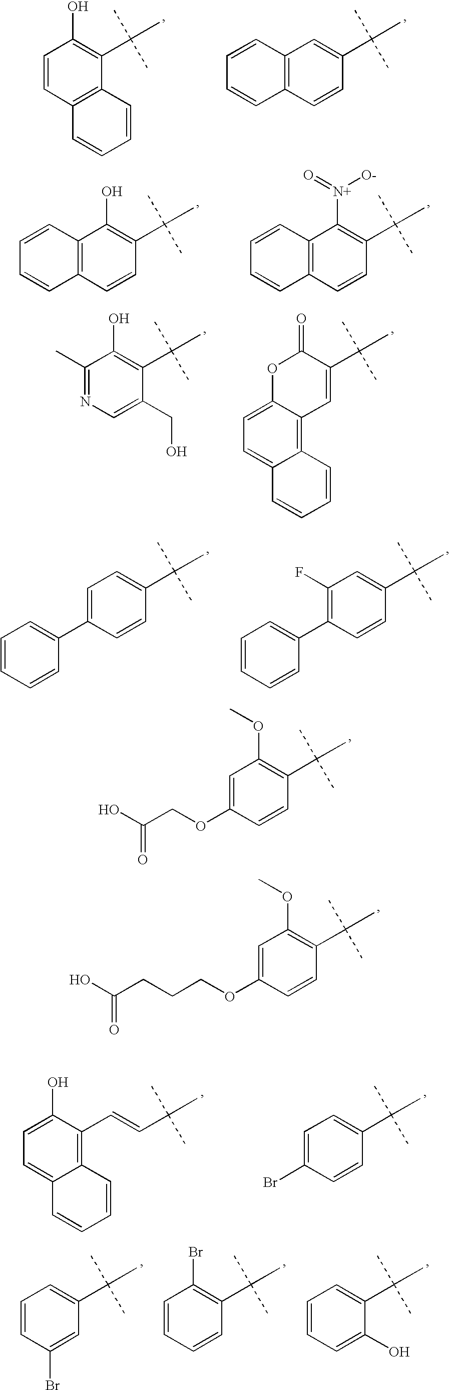 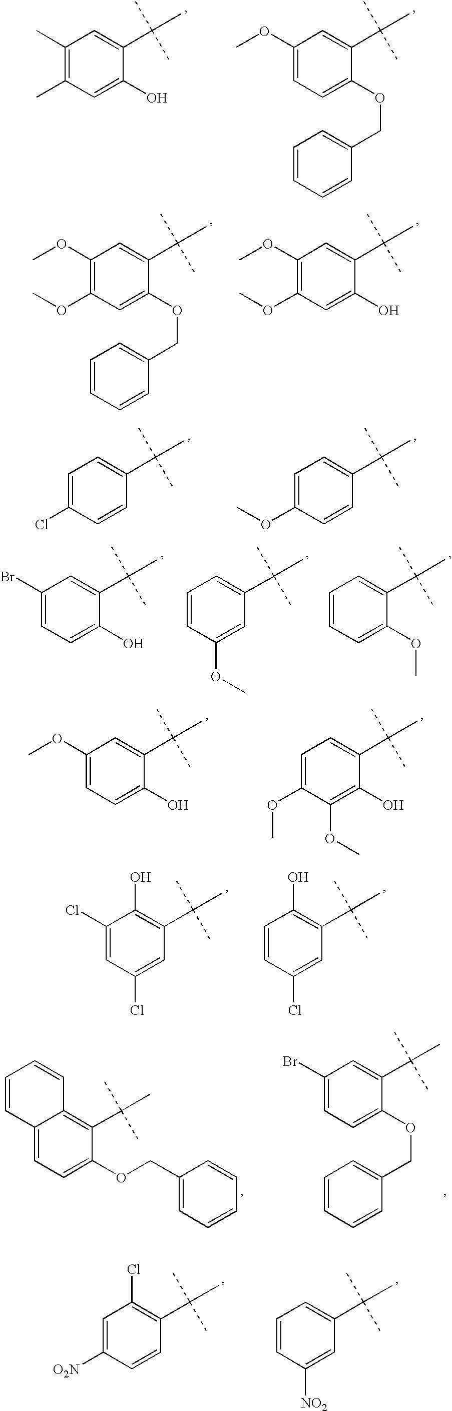 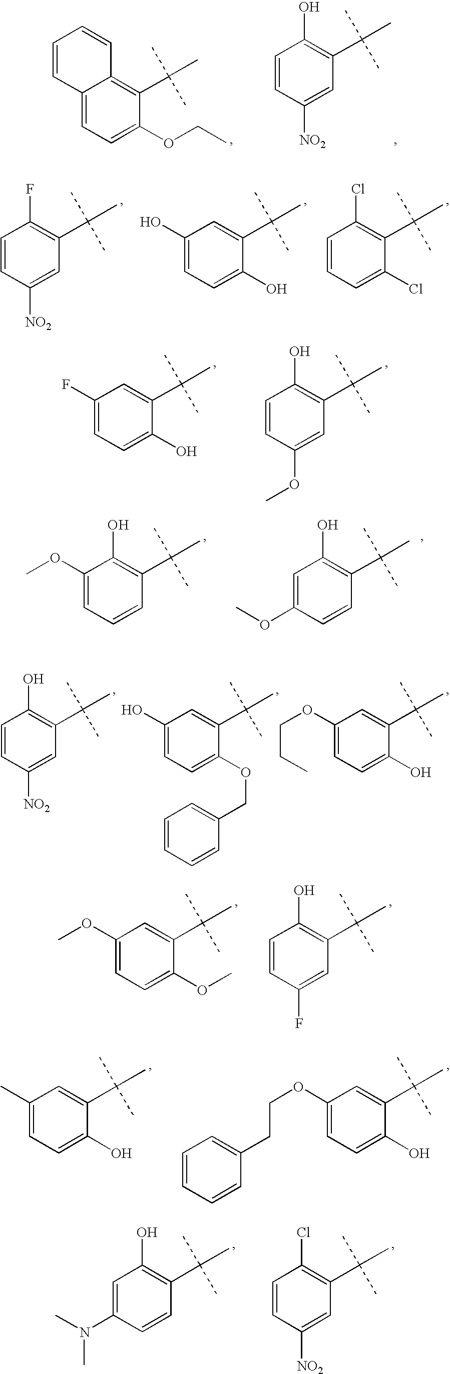 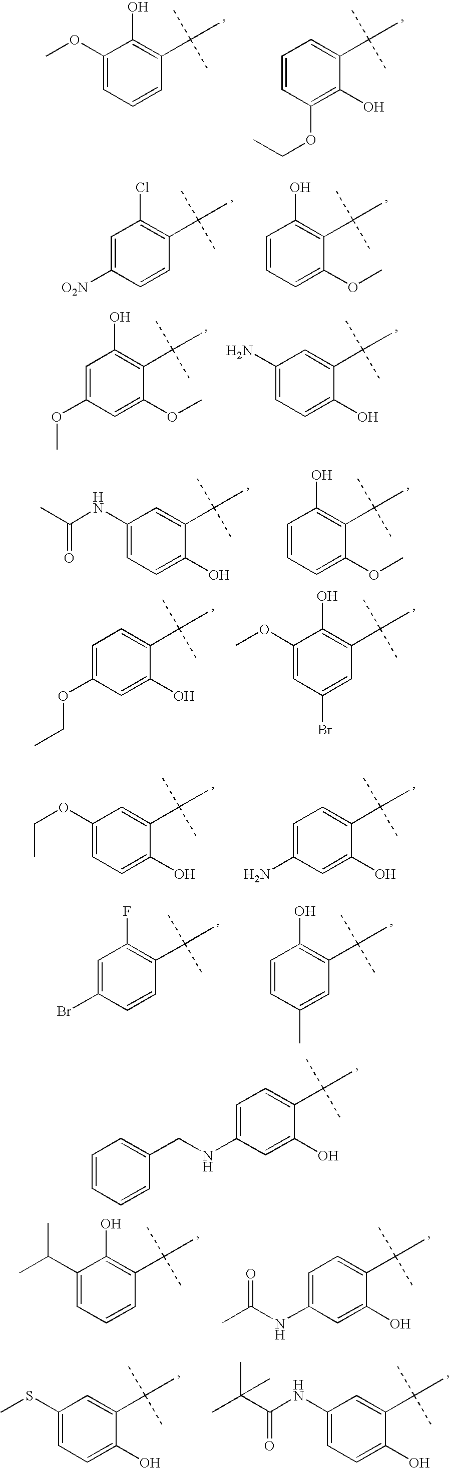 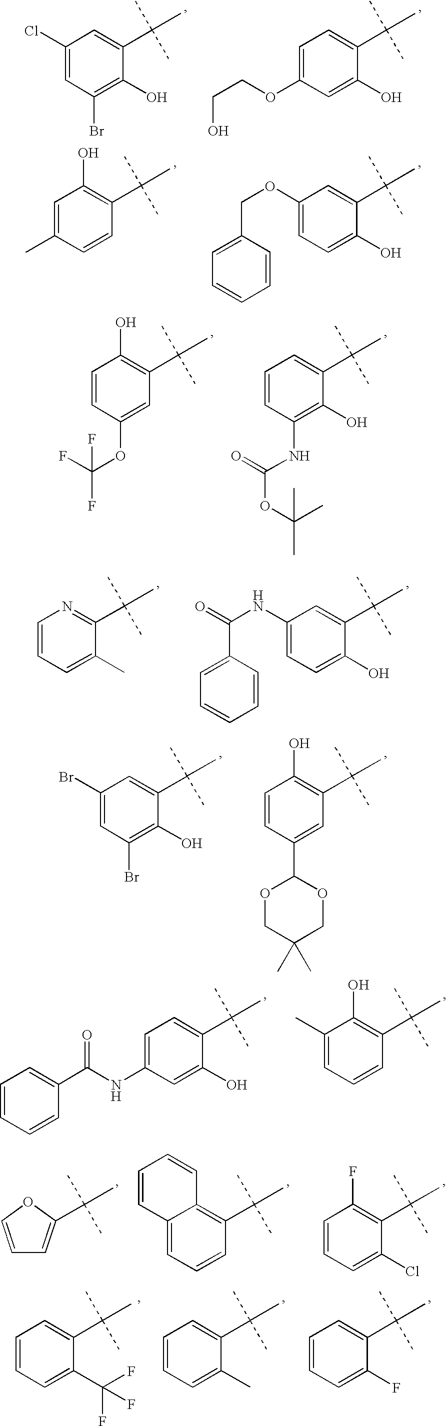 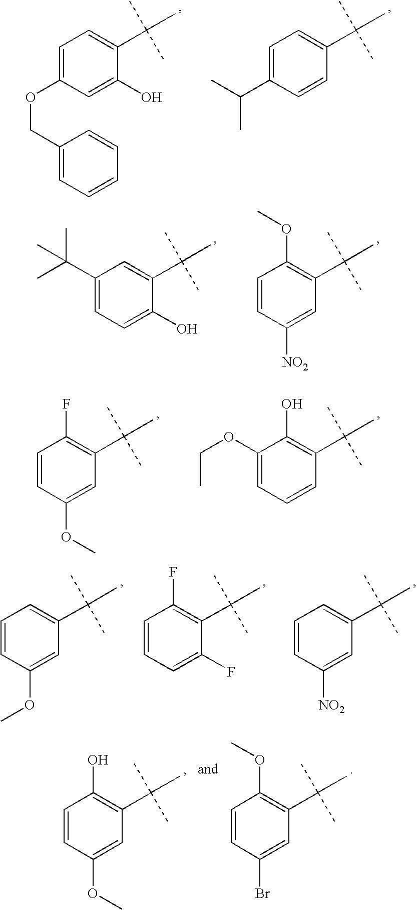
and Ring B is an unsubstituted or substituted aryl or heteroaryl selected from 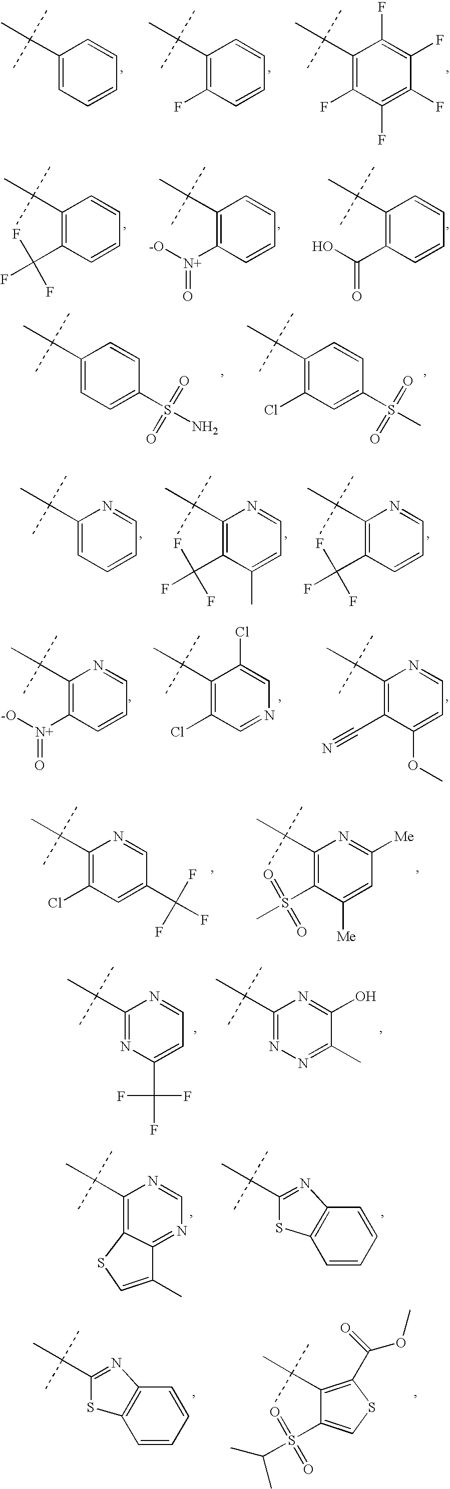 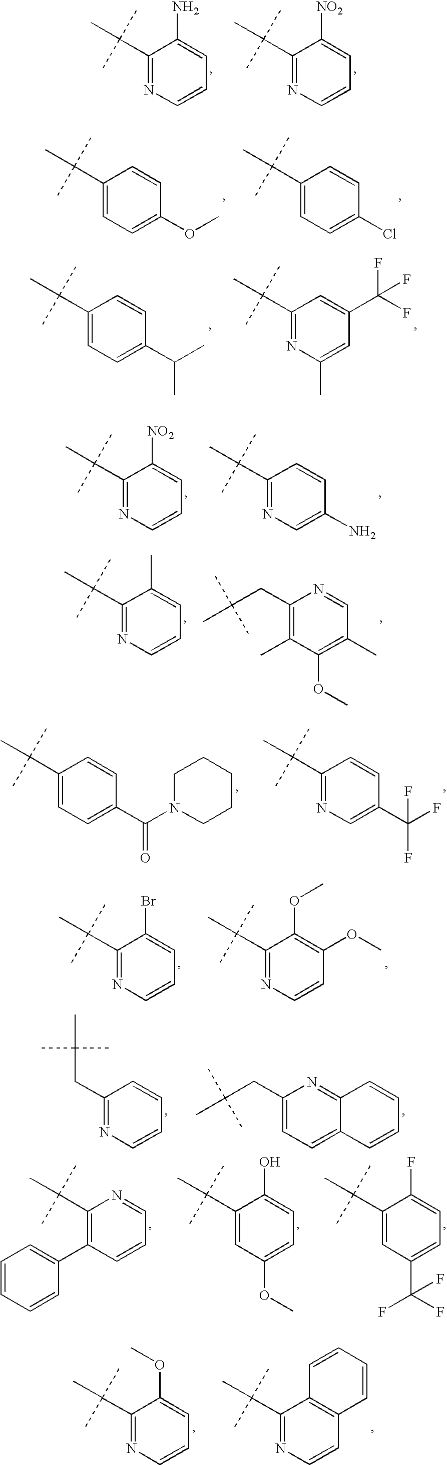 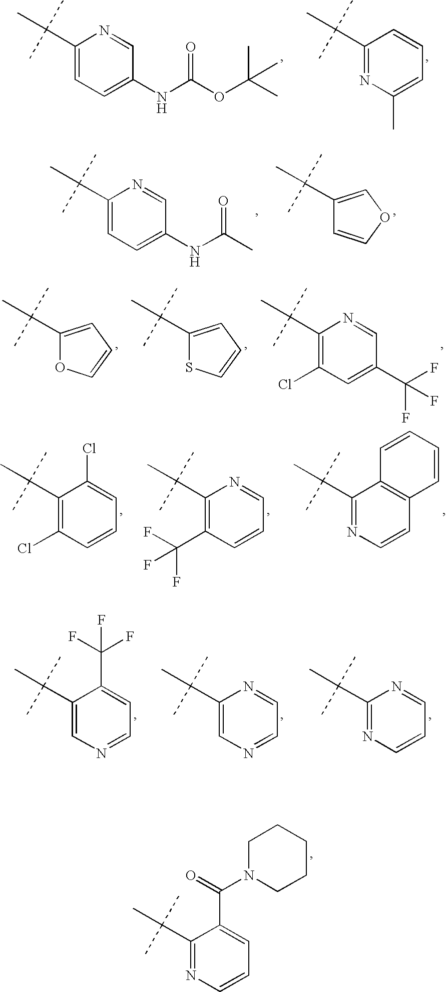
and

In another specific embodiment, the invention relates to compounds of Formula I selected from 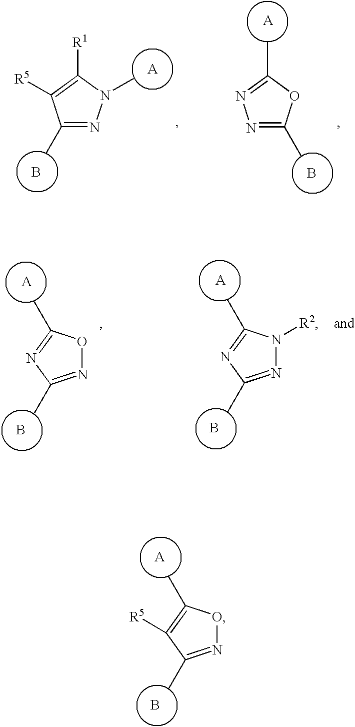
wherein R¹, R², R³, R⁴, and R⁵ are independently selected from hydrogen, halogen, or nitro, or an unsubstituted or substituted alkyl or alkoxy, and Ring A is an unsubstituted or substituted aryl, alkylaryl, heteroaryl, or alkylaryl selected from 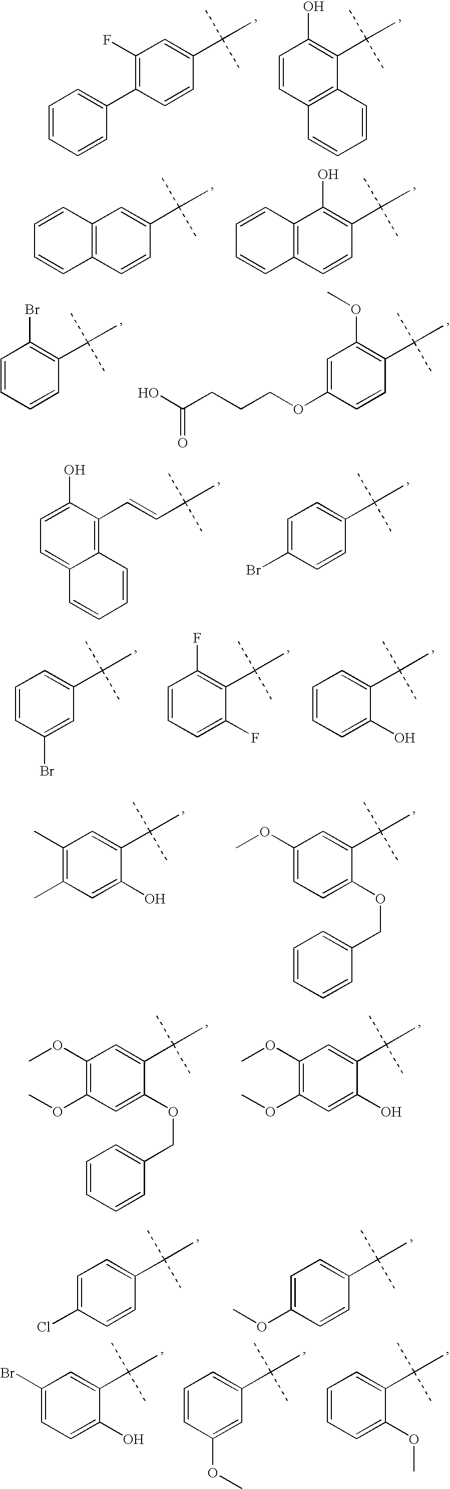 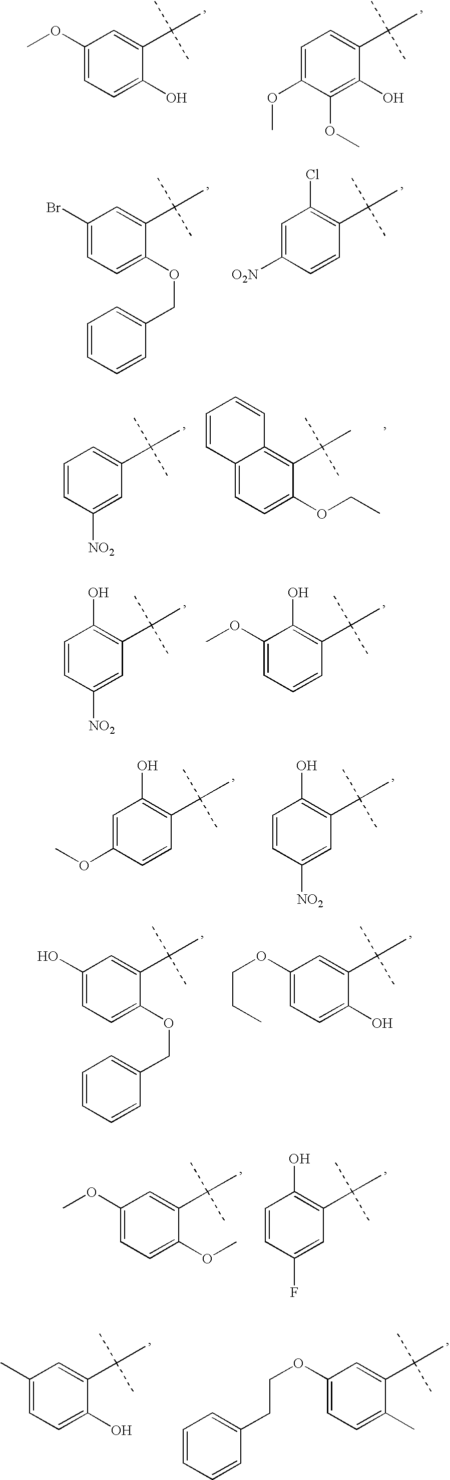 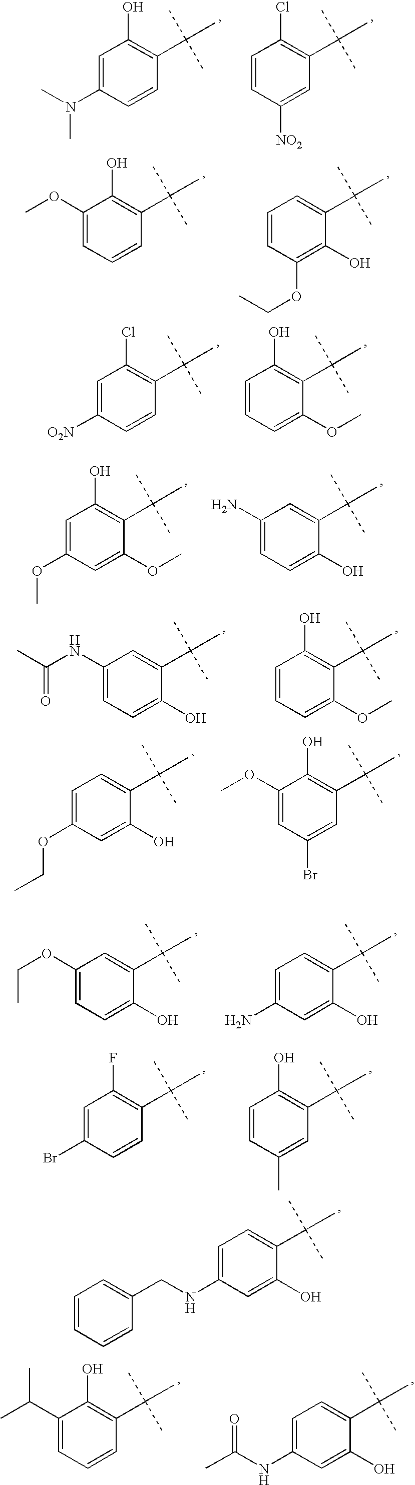 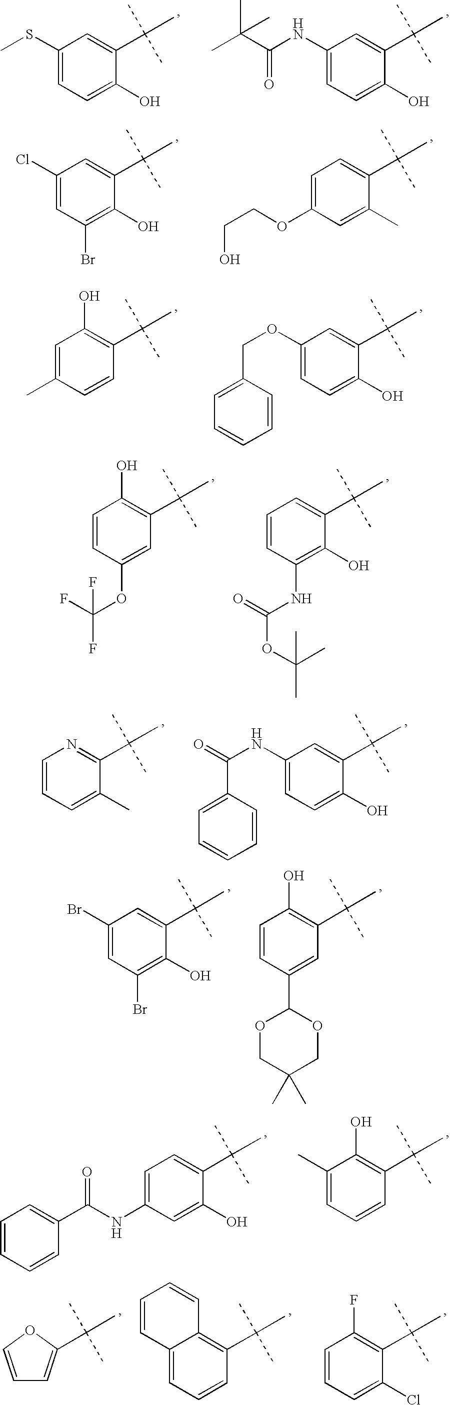 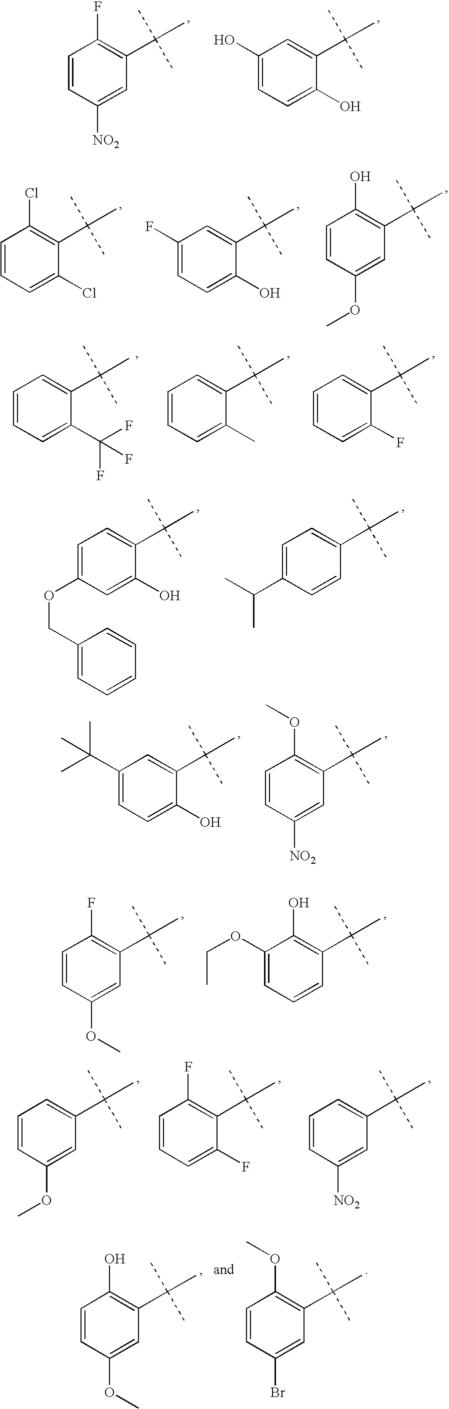
and Ring B is an unsubstituted or substituted heteroaryl selected from selected from

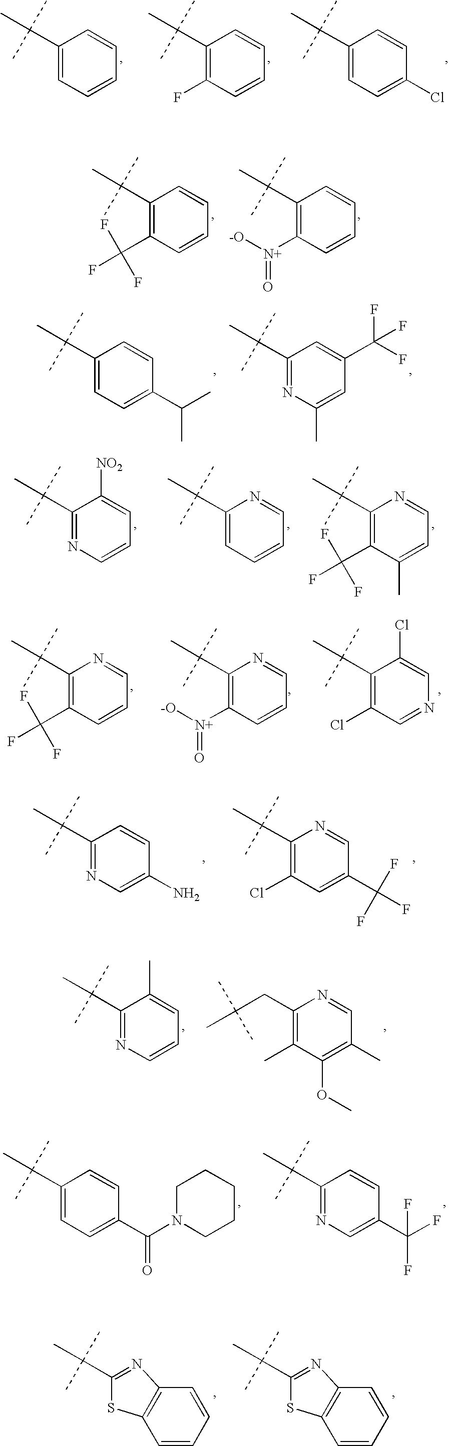

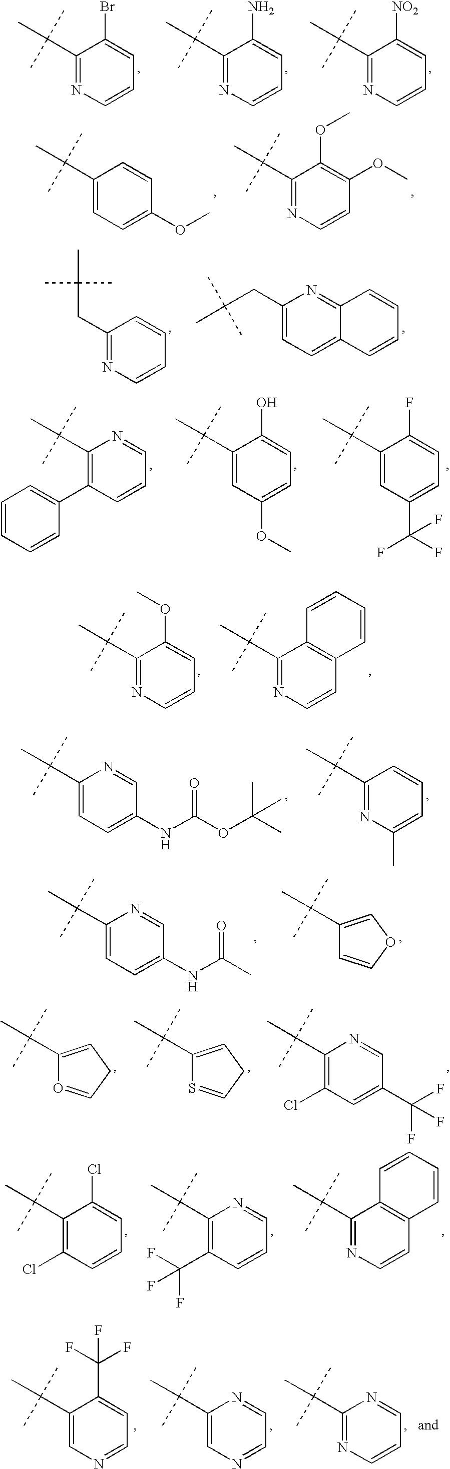

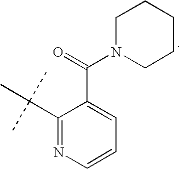

In one embodiment, the invention comprises a compound of Formula I wherein W is an N atom and wherein Y and Z form an unsaturated bond. In another embodiment, Z can be a C atom.

In another embodiment, the invention comprises a compound of Formula I wherein W is an O atom and wherein Y and Z form an unsaturated bond. In yet another embodiement, Z can be a C atom.

In a preferred embodiment, the invention comprises a compound of Formula I selected from the group consisting of:

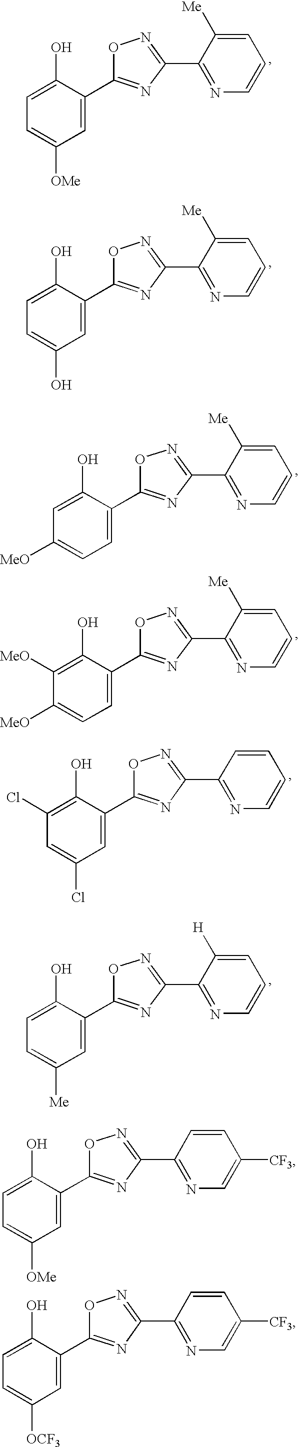

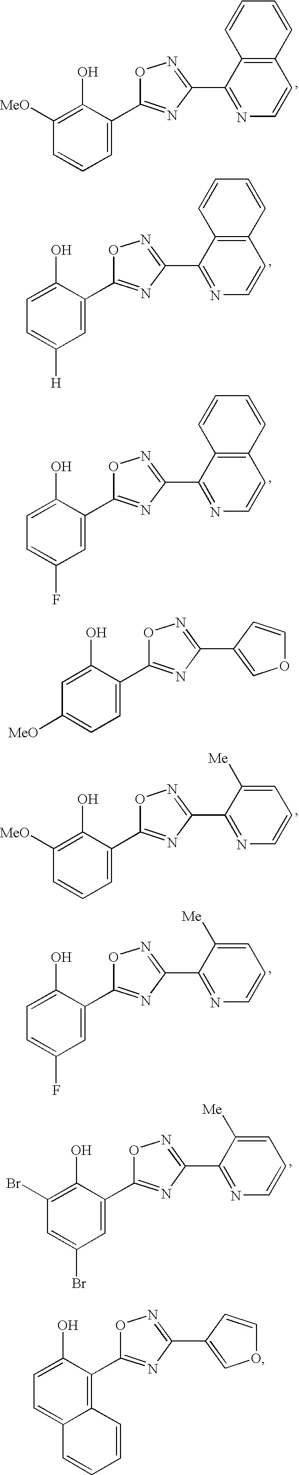

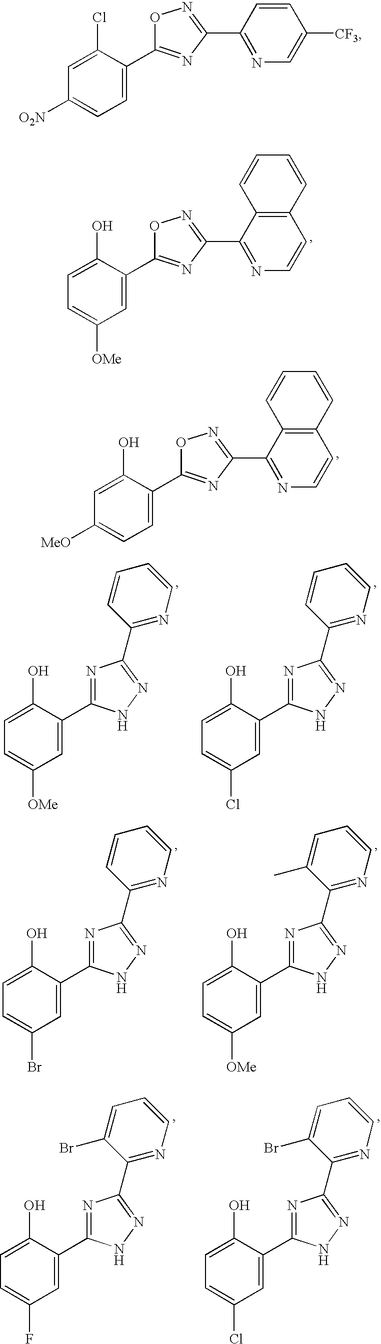

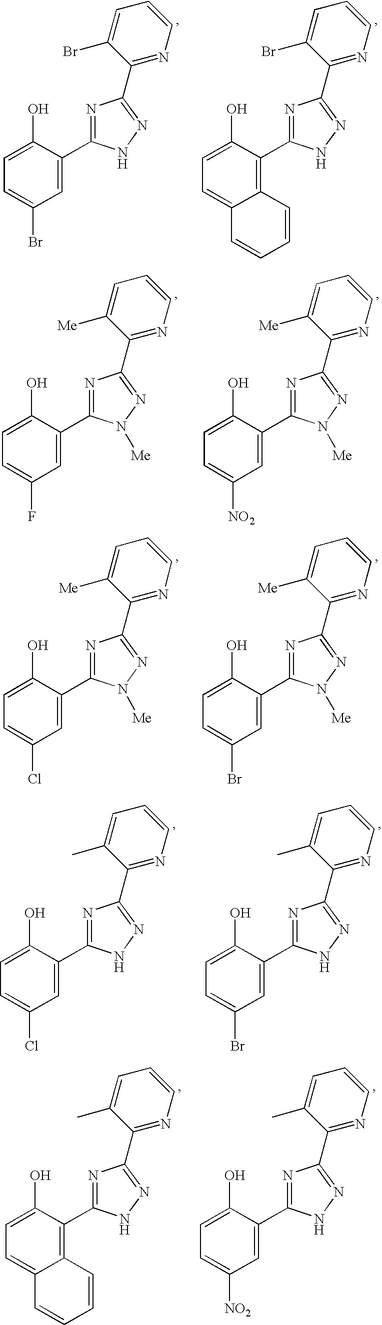

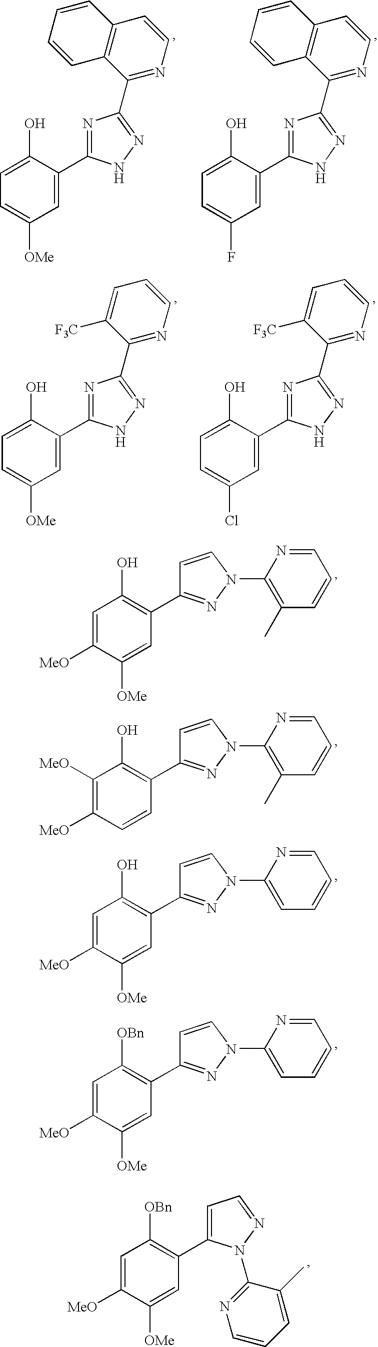

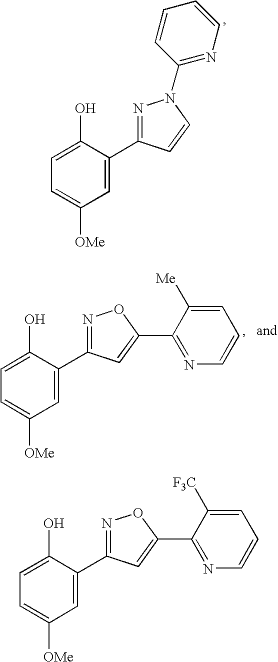

The invention is also directed to pharmaceutically acceptable prodrugs, pharmaceutically active metabolites, and pharmaceutically acceptable salts of the compounds, prodrugs, or metabolites of Formula I. Advantageous methods of making the compounds of Formula I are also described.

In another aspect, the invention relates to a method for the treatment hepatitis C virus in a mammal, including a human, comprising administering to said mammal an amount of a compound of the Formula I as defined above, or a pharmaceutically acceptable prodrug, pharmaceutically active metabolite, pharmaceutically acceptable solvate, or pharmaceutically acceptable salt, that is effect in treating hepatitis C virus. Advantageously, the mammal can be in need of the treatment. The compounds of the present invention inhibit HCV Internal Ribosome Entry Site (IRES)-specific translation activities. Since IRES-driven translation occurs in a number of other medically important RNA viruses, this approach offers the potential of identifying an antiviral agent with utility beyond HCV.

In another aspect, Formula I compounds or pharmaceutically acceptable compositions thereof are utilized in a method for treating the full range of viral diseases in mammals, including humans, by administering to the mammal a therapeutically effective amount of the compounds. Viral diseases contemplated to be treated with Formula I compounds include acute and chronic infections caused by RNA viruses. Without limiting in any way the range of viral infections that may be treated, compounds of Formulas I are particularly useful in the treatment of infections caused by flaviviruses including hepatitis C virus, yellow fever virus, west Nile virus, Dengue virus, and Tick-borne encephalitis; picornaviruses including poliovirus, coxsackievirus, enterovirus, and rhinovirus; and togaviruses including rubellavirus; and nidoviruses including SARS-Coronavirus.

### DETAILED DESCRIPTION OF THE INVENTION AND PREFERRED EMBODIMENTS

Where the following terms are used in this specification, they are used as defined below:

The terms “comprising” and “including” are used herein in their open, non-limiting sense.

The term “alkyl” as used herein refers to a straight- or branched-chain alkyl group having one to twelve carbon atoms. Exemplary alkyl groups include methyl (Me, which also may be structurally depicted by “/”), ethyl (Et), n-propyl, isopropyl, butyl, isobutyl, sec-butyl, tert-butyl (tBu), pentyl, isopentyl, tert-pentyl, hexyl, isohexyl, and the like.

The term “alkoxy” refers to —O-alkyl. Illustrative examples include methoxy, ethoxy, propoxy, and the like.

The term “alkenyl” represents alkyl moieties having at least one carbon-carbon double bond wherein alkyl is as defined above and including E and Z isomers of said alkenyl moiety.

The term “halogen” represents chlorine, fluorine, bromine or iodine. The term “halo” represents chloro, fluoro, bromo or iodo.

The term “cycloalkyl” refers to a saturated or partially saturated, monocyclic or fused or spiro polycyclic, carbocycle having from three to twelve ring atoms per ring. Illustrative examples of cycloalkyl groups include the following moieties: 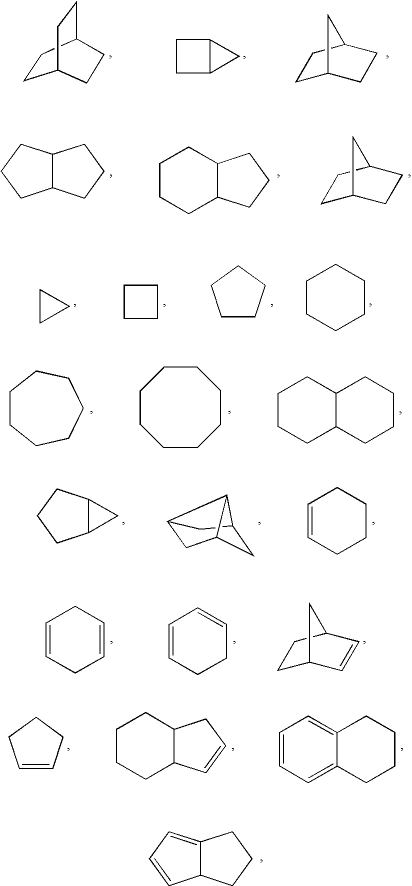
and the like.

A “heterocycloalkyl” refers to a monocyclic, or fused or spiro polycyclic, ring structure that is saturated or partially saturated and has from three to twelve ring atoms per ring selected from C atoms and N, O, and S heteroatoms. Illustrative examples of heterocycloalkyl groups include: 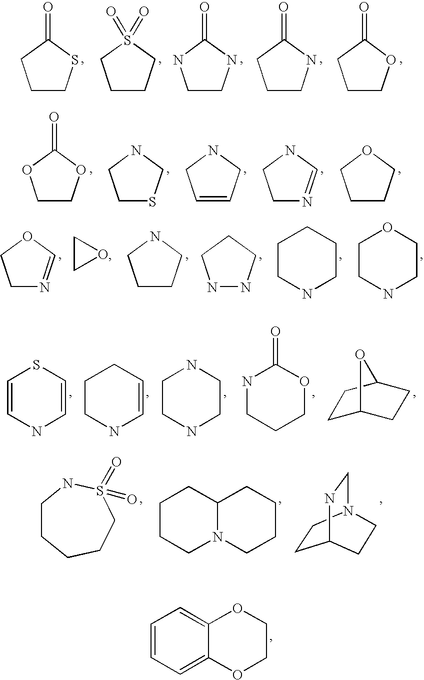
and the like.

The term “aryl” (Ar) refers to a monocyclic, or fused or Spiro polycyclic, aromatic carbocycle (ring structure having ring atoms that are all carbon) having from three to twelve ring atoms per ring. Illustrative examples of aryl groups include the following moieties: 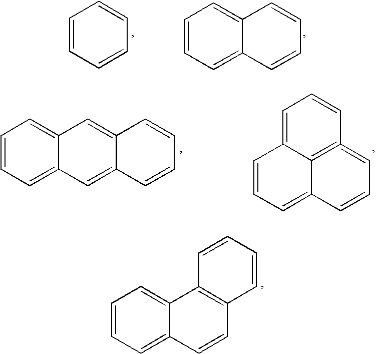
and the like.

The term alkylaryl as used herein refers to a straight- or branched-chain alkyl group having one to twelve carbon atoms substituted with one or more aryl groups.

The term “heteroaryl” (heteroAr) refers to a monocyclic, or fused or Spiro polycyclic, aromatic heterocycle (ring structure having ring atoms selected from carbon atoms as well as nitrogen, oxygen, and sulfur heteroatoms) having from three to twelve ring atoms per ring. Illustrative examples of aryl groups include the following moieties: 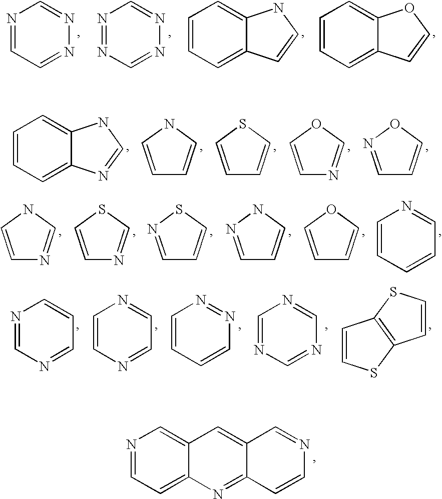
and the like.

The term alkylheteroaryl as used herein refers to a straight- or branched-chain alkyl group having one to twelve carbon atoms substituted with one or more heteroaryl groups.

The term “substituted” means that the specified group or moiety bears one or more substituents. The term “unsubstituted” means that the specified group bears no substituents.

A substituted alkyl, alkoxy, alkenyl, cycloalkyl, heterocycloalkyl, aryl, alkylaryl, heteroaryl, or alkylheteroaryl is substituted by one or more substituents including halogen (F, Cl, Br, or I), lower alkyl (C₁₋₆), —OH, —NO₂, —CN, —CO₂H, —O-lower alkyl, cydoalkyl, heterocycloalkyl-aryl, -aryl-lower alkyl, —CO₂CH₃, —CONH₂, —CONH(alkyl), —CONH(aryl), —CONH(heteroaryl), —OCH₂CONH₂, —NH₂, —NH(alkyl), —NH(aryl), —NH(heteroaryl), —SO₂NH₂, haloalkyl (e.g., —CF₃, —CH₂CF₃), and —O-haloalkyl (e.g., —OCF₃, —OCHF₂), wherein the alkyl, cydoalkyl, heterocycloalkyl aryl, and heteroaryl groups are optionally substituted by one or more substituents including halogen (F, Cl, Br, or I), lower alkyl (C₁₋₆), —OH, —NO₂, —CN, —CO₂H, —O-lower alkyl, -aryl, -aryl-lower alkyl, —CO₂CH₃, —CONH₂, —CONH(alkyl), —CONH(aryl), —CONH(heteroaryl), —OCH₂CONH₂, —NH₂, —NH(alkyl), —NH(aryl), —NH(heteroaryl), —SO₂NH₂, haloalkyl (e.g., —CF₃, —CH₂CF₃), and —O-haloalkyl (e.g., —OCF₃, —OCHF₂).

In accordance with a convention used in the art, 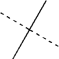
is used in structural formulae herein to depict the bond that is the point of attachment of the moiety or substituent to the core or backbone structure. Moreover, 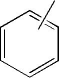
is used in structural formulae herein to depict that the point of attachment of the moiety or substituent to the core of the backbone aryl structure is unspecified.

The term “preventing” refers to the ability of a compound or composition of the invention to prevent a disease identified herein in patients diagnosed as having the disease or who are at risk of developing such disease. The term also encompasses preventing further progression of the disease in patients who are already suffering from or have symptoms of such disease.

The term “treating” refers to:

(i) preventing a disease, disorder, or condition from occurring in an animal or human that may be predisposed to the disease, disorder and/or condition, but has not yet been diagnosed as having it;

(ii) inhibiting the disease, disorder, or condition, i.e., arresting its development; and

(iii) relieving the disease, disorder, or condition, i.e., causing regression of the disease, disorder, and/or condition.

The term “treatment” refers to the act of treating as “treating” is defined immediately above.

The compounds of the invention may exhibit the phenomenon of tautomerism. While Formula I cannot expressly depict all possible tautomeric forms, it is to be understood that Formula I is intended to represent any tautomeric form of the depicted compound and are not to be limited merely to a specific compound form depicted by the formula drawings.

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, Formula I is intended to cover solvated as well as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formula I, the invention includes pharmaceutically acceptable prodrugs, pharmaceutically active metabolites, and pharmaceutically acceptable salts of such compounds and metabolites.

“A pharmaceutically acceptable prodrug” is a compound that may be converted under physiological conditions or by solvolysis to the specified compound or to a pharmaceutically acceptable salt of such compound prior to exhibiting its pharmacological effect (s). Typically, the prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side-effects (e.g., toxicity). The prodrug can be readily prepared from the compounds of Formulas I by using methods known in the art, such as those described by Burger's Medicinal Chemistry and Drug Chemistry, 1, 172-178, 949-982 (1995). See also Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

“A pharmaceutically active metabolite” is intended to mean a pharmacologically active product produced through metabolism in the body of a specified compound or salt thereof. After entry into the body, most drugs are substrates for chemical reactions that may change their physical properties and biologic effects. These metabolic conversions, which usually affect the polarity of the Formula I compounds, alter the way in which drugs are distributed in and excreted from the body. However, in some cases, metabolism of a drug is required for therapeutic effect. For example, anticancer drugs of the anti-metabolite class must be converted to their active forms after they have been transported into a cancer cell.

Since most drugs undergo metabolic transformation of some kind, the biochemical reactions that play a role in drug metabolism may be numerous and diverse. The main site of drug metabolism is the liver, although other tissues may also participate.

A feature characteristic of many of these transformations is that the metabolic products, or “metabolites,” are more polar than the parent drugs, although a polar drug does sometime yield a less polar product. Substances with high lipid/water partition coefficients, which pass easily across membranes, also diffuse back readily from tubular urine through the renal tubular cells into the plasma. Thus, such substances tend to have a low renal clearance and a long persistence in the body. If a drug is metabolized to a more polar compound, one with a lower partition coefficient, its tubular reabsorption will be greatly reduced. Moreover, the specific secretory mechanisms for anions and cations in the proximal renal tubules and in the parenchymal liver cells operate upon highly polar substances.

As a specific example, phenacetin (acetophenetidin) and acetanilide are both mild analgesic and antipyretic agents, but are transformed within the body to a more polar and more effective metabolite, p-hydroxyacetanilid (acetaminophen), which is widely used today. When a dose of acetanilide is given to a person, the successive metabolites peak and decay in the plasma sequentially. During the first hour, acetanilide is the principal plasma component. In the second hour, as the acetanilide level falls, the metabolite acetaminophen concentration reaches a peak. Finally, after a few hours, the principal plasma component is a further metabolite that is inert and can be excreted from the body. Thus, the plasma concentrations of one or more metabolites, as well as the drug itself, can be pharmacologically important.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both finctional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fuimaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

A further aspect of the present invention is directed to a pharmaceutical composition comprising a pharmaceutically acceptable carrier or a diluent and a therapeutically effective amount of a Formula I compound, a pharmaceutically acceptable salt, hydrate, ester, solvate, prodrug, metabolite, or stereoisomer.

Formula I compounds are useful in the manufacture of pharmaceutical formulations comprising an effective amount thereof in conjunction with or as an admixture with excipients or carriers suitable for either enteral or parenteral application. As such, formulations of the present invention suitable for oral administration may be in the form of discrete units such as capsules, cachets, tablets, troche or lozenges, each containing a predetermined amount of the active ingredient; in the form of a powder or granules; in the form of a solution or a suspension in an aqueous liquid or nonaqueous liquid; or in the form of an oil-in-water emulsion or a water-in-oil emulsion. The active ingredient may also be in the form of a bolus, electuary, or paste.

The composition will usually be formulated into a unit dosage form, such as a tablet, capsule, aqueous suspension or solution. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

Particularly preferred formulations include tablets and gelatin capsules comprising the active ingredient together with (a) diluents, such as lactose, dextrose, sucrose, mannitol, sorbitol, cellulose, dried corn starch, and glycine; and/or (b) lubricants, such as silica, talcum, stearic acid, its magnesium or calcium salt, and polyethylene glycol.

Tablets may also contain binders, such as magnesium aluminum silicate, starch paste, gelatin, tragacanth, methylcellulose, sodium carbosymethylcellulose and polyvinylpyrrolidone; carriers, such as lactose and corn starch; disintegrants, such as starches, agar, alginic acid or its sodium salt, and effervescent mixtures; and/or absorbents, colorants, flavors, and sweeteners. The compositions of the invention may be sterilized and/or contain adjuvants, such as preserving, stabilizing, swelling or emulsifying agents, solution promoters, salts for regulating osmotic pressure, and/or buffers. In addition, the composition may also contain other therapeutically valuable substances. Aqueous suspensions may contain emulsifying and suspending agents combined with the active ingredient. All oral dosage forms may further contain sweetening and/or flavoring and/or coloring agents.

These compositions are prepared according to conventional mixing, granulating, or coating methods, respectively, and contain about 0.1 to 75% of the active ingredient, preferably about 1 to 50% of the same. A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding, in a suitable machine, a mixture of the powdered active ingredient and a suitable carrier moistened with an inert liquid diluent.

When administered parenterally, the composition will normally be in a unit dosage, sterile injectable form (aqueous isotonic solution, suspension, or emulsion) with a pharmaceutically acceptable carrier. Such carriers are preferably non-toxic, parenterally-acceptable and contain non-therapeutic diluents or solvents. Examples of such carriers include water; aqueous solutions, such as saline (isotonic sodium chloride solution), Ringer's solution, dextrose solution, and Hanks' solution; and nonaqueous carriers, such as 1,3-butanediol, fixed oils (e.g., corn, cottonseed, peanut, sesame oil, and synthetic mono- or di-glyceride), ethyl oleate, and isopropyl myristate.

Oleaginous suspensions can be formulated according to techniques known in the art using suitable dispersing or wetting agents and suspending agents. Among the acceptable solvents or suspending mediums are sterile fixed oils. For this purpose, any bland fixed oil may be used. Fatty acids, such as oleic acid and its glyceride derivatives, including olive oil and castor oil, especially in their polyoxyethylated forms, are also useful in the preparation of injectables. These oil solutions or suspensions may also contain long-chain alcohol diluents or dispersants.

Sterile saline is a preferred carrier, and the compounds are often sufficiently water soluble to be made up as a solution for all foreseeable needs. The carrier may contain minor amounts of additives, such as substances that enhance solubility, isotonicity, and chemical stability, e.g., anti-oxidants, buffers and preservatives.

When administered rectally, the composition will usually be formulated into a unit dosage form such as a suppository or cachet. These compositions can be prepared by mixing the compound with suitable non-irritating excipients that are solid at room temperature, but liquid at rectal temperature, such that they will melt in the rectum to release the compound. Common excipients include cocoa butter, beeswax and polyethylene glycols or other fatty emulsions or suspensions.

Formulations suitable for nasal or buccal administration (such as self-propelling powder dispensing formulations), may comprise about 0.1% to about 5% w/w of the active ingredient or, for example, about 1% w/w of the same. In addition, some formulations can be compounded into a sublingual troche or lozenge.

Moreover, the compounds may be administered topically, especially when the conditions addressed for treatment involve areas or organs readily accessible by topical application, including disorders of the eye, the skin or the lower intestinal tract.

For topical application to the eye, or ophthalmic use, the compounds can be formulated as micronized suspensions in isotonic, pH-adjusted sterile saline or, preferably, as a solution in isotonic, pH-adjusted sterile saline, either with or without a preservative such as benzylalkonium chloride. Alternatively, the compounds may be formulated into ointments, such as petrolatum.

For topical application to the skin, the compounds can be formulated into suitable ointments containing the compounds suspended or dissolved, for example, mixtures with one or more of the following: mineral oil, liquid petrolatum, white petrolatum, propylene glycol, polyoxyethylene compound, polyoxypropylene compound, emulsifying wax and water. Alternatively, the compounds can be formulated into suitable lotions or creams containing the active compound suspended or dissolved in, for example, a mixture of one or more of the following: mineral oil, sorbitan monostearate, polysorbate 60, cetyl ester wax, cetearyl alcohol, 2-octyldodecanol, benzyl alcohol and water.

Topical application to the lower intestinal tract can be effected in rectal suppository formulations (see above) or in suitable enema formulations.

The formulations may conveniently be presented in unit dosage form and may be prepared by any of the methods well known in the art of pharmacy. All methods include the step of bringing the active ingredient into association with the carrier, which constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

The pharmaceutical composition of the present invention is used in amount that are therapeutically effective and the amounts used may depend upon the desire release profile, the concentration of the pharmaceutical composition required for the sensitizing effect, and the length of time that the pharmaceutical composition has to be released for treatment.

Formula I compounds of the invention are preferably administered as a capsule or tablet containing a single or divided dose of the compound, or as a sterile solution, suspension, or emulsion, for parenteral administration in a single or divided dose.

The compounds of the invention are used in the composition in amounts that are therapeutically effective. While the effective amount of the Formula I compounds will depend upon the particular compound being used, amounts of these compounds varying from about 1% to about 65% have been easily incorporated into liquid or solid carrier delivery systems.

For medical use, the amount required of a Formula I compound to achieve a therapeutic effect will vary according to the particular compound administered, the route of administration, the mammal under treatment, and the particular disorder in disease concerned. A suitable systemic dose of a Formula I compound for a mammal suffering from, or likely to suffer from, any condition as described herein is typically in the range of about 0.1 to about 100 mg of base per kilogram of body weight. It is understood that the ordinarily skilled physician or veterinarian will readily be able to determine and prescribe the amount of the compound effective for the desired prophylactic or therapeutic treatment.

In so proceeding, the physician or veterinarian may employ an intravenous bolus followed by an intravenous infusion and repeated administrations, as considered appropriate. In the methods of the present invention, the compounds may be administered, for example, orally, parentally, in inhalation spray, topically, rectally, nasally, buccally, sublingually, vaginally, intraventricularly, or via an implanted reservoir in dosage formulations containing conventional non-toxic pharmaceutically-acceptable carriers, adjuvants and vehicles.

Parenteral includes, but is not limited to, the following examples of administration: intravenous, subcutaneous, intramuscular, intraspinal, intraosseous, intraperitoneal, intrathecal, intraventricular, intrastemal or intracranial injection and infusion techniques, such as by subdural pump. Invasive techniques are preferred, particularly direct administration to damaged neuronal tissue. While it is possible for the Formula I compounds to be administered alone, it is preferable to provide it as part of a pharmaceutical formulation.

To be effective therapeutically as central nervous system targets, the compounds used in the methods of the present invention should readily penetrate the blood-brain barrier when peripherally administered. Compounds that cannot penetrate the blood-brain barrier, however, can still be effectively administered by an intraventricular route.

The compounds used in the methods of the present invention may be administered by a single dose, multiple discrete doses or continuous infusion. Since the compounds are small, easily diffusible and relatively stable, they are well suited to continuous infusion. Pump means, particularly subcutaneous or subdural pump means, are preferred for continuous infusion.

For the methods of the present invention, any effective administration regimen regulating the timing and sequence of doses may be used. Doses of the compounds preferably include pharmaceutical dosage units comprising an efficacious quantity of active compound. By an efficacious quantity is meant a quantity sufficient to provide immune enhancing response and/or derive the desired beneficial effects through administration of one or more of the pharmaceutical dosage units.

An exemplary daily dosage unit for a vertebrate host comprises an amount of from about 0.001 mg/kg to about 50 mg/kg. Typically, dosage levels on the order of about 0.1 mg to about 10,000 mg of the active ingredient compound are useful in the treatment of the above conditions, with preferred levels being about 0.5 mg to about 2,000 mg. The specific dose level for any particular patient will vary depending upon a variety of factors, including the activity of the specific compound employed; the age, body weight, general health, sex, and diet of the patient; the time of administration; the rate of excretion, any combination of the compound with other drugs; the severity of the particular disease being treated; and the form and route of administration. Typically, in vitro dosage-effect results provide useful guidance on the proper doses for patient administration. Studies in animal models can also be helpful. The considerations for determining the proper dose levels are well known in the art.

The compounds and compositions can be co-administered with one or more therapeutic agents either (i) together in a single formation, or (ii) separately in individual formulations designed for optimal release rates of their respective active agent. Each formulation may contain from about 0.01% to about 99.99% by weight, preferably from about 3.5% to about 60% by weight, of the compound of the invention, as well as one or more pharmaceutical excipients, such as wetting, emulsifying and pH buffering agents. When the compounds used in the methods of the invention are administered in combination with one or more other therapeutic agents, specific dose levels for those agents will depend upon considerations such as those identified above for compositions and methods of the invention in general.

For the methods of the present invention, any administration regimen regulating the timing and sequence of delivery of the compound can be used and repeated as necessary to effect treatment. Such regimen may include pretreatment and/or co-administration with additional therapeutic agents.

The inventive agents may be prepared using the reaction routes and synthesis schemes as described below, employing the general techniques known in the art using starting materials that are readily available. The synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

Preparation of Compounds

In the synthetic schemes described below, unless otherwise indicated all temperatures are set forth in degrees Celsius and all parts and percentages are by weight. Reagents were purchased from commercial suppliers such as Aldrich Chemical Company or Lancaster Synthesis Ltd. and were used without further purification unless otherwise indicated. Tetrahydrofuran (THF) and N, N-dimethylforamide (DMF) were purchased from Aldrich in Sure Seal bottles and used as received. Unless otherwise indicated, the following solvents and reagents were distilled under a blanket of dry nitrogen. THF, and Et₂O were distilled from Na-benzophenone ketyl; CH₂Cl₂, diisopropylamine, pyridine and Et₃N were distilled from CaH₂; MeCN was distilled first from P₂O₅, then from CaH₂; MeOH was distilled from Mg; PhMe, EtOAc and i-PrOAc were distilled from CaH₂; TFAA was purified via simple atmospheric distillation under dry argon.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried. The reactions were assayed by TLC and terminated as judged by the consumption of starting material. Analytical thin layer chromatography (TLC) was performed on aluminum-backed silica gel 60 F₂₅₄ 0.2 mm plates (EM Science), and visualized with UV light (254 mn) followed by heating with commercial ethanolic phosphomolybdic acid. Preparative thin layer chromatography (TLC) was performed on aluminum-backed silica gel 60 F₂₅₄ 1.0 mm plates (EM Science) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or Mg₂SO₄ prior to filtration and evaporation of the solvents under reduced pressure on a rotary evaporator and noted as solvents removed in vacuo. Column chromatography was completed under positive pressure using 230-400 mesh silica gel or 50-200 mesh neutral alumina. Hydrogenolysis was done at the pressure indicated in the examples or at ambient pressure.

¹H-NMR spectra were recorded on a Varian Mercury-VX400 instrument operating at 400 MHz and ¹³C-NMR spectra were recorded operating at 75 MHz. NMR spectra were obtained as CDCl₃ solutions (reported in ppm), using chloroform as the reference standard (7.27 ppm and 77.00 ppm), CD₃OD (3.4 and 4.8 ppm and 49.3 ppm), DMSO-d₆, or internally tetramethylsilane (0.00 ppm) when appropriate. Other NMR solvents were used as needed. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

Infrared (IR) spectra were recorded on a FT-IR Spectrometer as neat oils, as KBr pellets, or as CDCl₃ solutions, and when given are reported in wave numbers (cm⁻¹). Mass spectra reported are (+)-ES LC/MS conducted by the Analytical Chemistry Department of Anadys Pharmaceuticals, Inc. Elemental analyses were conducted by the Atlantic Microlab, Inc. in Norcross, Ga. Melting points (mp) were determined on an open capillary apparatus, and are uncorrected.

The described synthetic pathways and experimental procedures utilize many common chemical abbreviations, THF (tetrahydrofuran), DMF (N,N-dimethylformamide), EtOAc (ethyl acetate), DMSO (di-methyl sulfoxide), DMAP (4-dimethylaminopyridine), DBU (1,8-diazacyclo[5.4.0]undec-7-ene), DCM (4-(dicyanomethylene)-2-methyl-6-(4-dimethylamino-styryl)-4 H-pyran), TFAA (trifluoroacetic anhydride), pyBOP (benzotriazol-1-yloxy)tripyrrolidinophosphonium hexafluorophosphate), DIEA (diisopropylethylamine), and the like.

After evaporation of solvents, the compounds of Formula I are purified by HPLC if needed.

The following are representative examples of Formula I compounds.

Pyrazoles

Scheme 1 shows a general procedure to prepare the 1,2- and 1,3-disubstituted pyrazoles from the corresponding arylmethylketones.

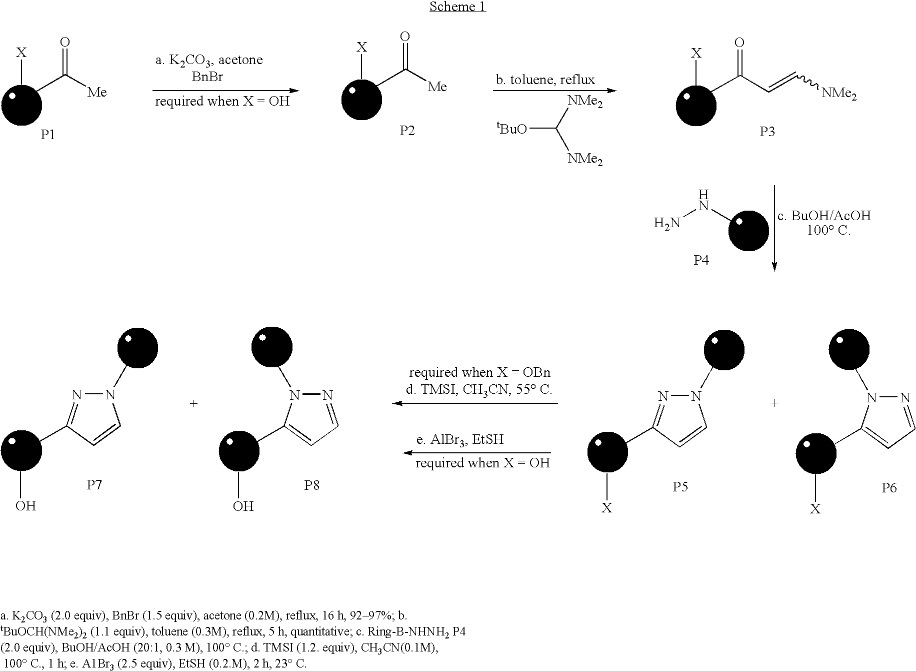

In a typical synthetic route, the o-hydroxyl arylmethyl ketone P1 is first protected as the corresponding benzyl-ether as shown in scheme 1. Reaction with tert-bulylbis-(dimethylamino)methane in refluxing toluene furnished masked ketoaldehyde P3 in quantitative yield. The desired protected 1,3- and 1,2-pyrazoles, P5 and P6 respectively, resulted from treatment of the above intermediate with a variety of arylhydrazines P4 in a butanol/acetic acid solvent system. The final products P7 and P8 were obtained after the removal of the benzyl protecting group by trimethylsilyl iodide and chromatographic separation. A modification of the above sequence, that involves the replacement of the final deprotection by treatment with AlBr₃ and EtSH, is employed when the commercially available methyl ethers were utilized in the same synthetic transformations.

Scheme 2 shows a general procedure to prepare the 1,2- and 1,3-disubstituted pyrazoles from the corresponding monosubstituted pyrazoles.

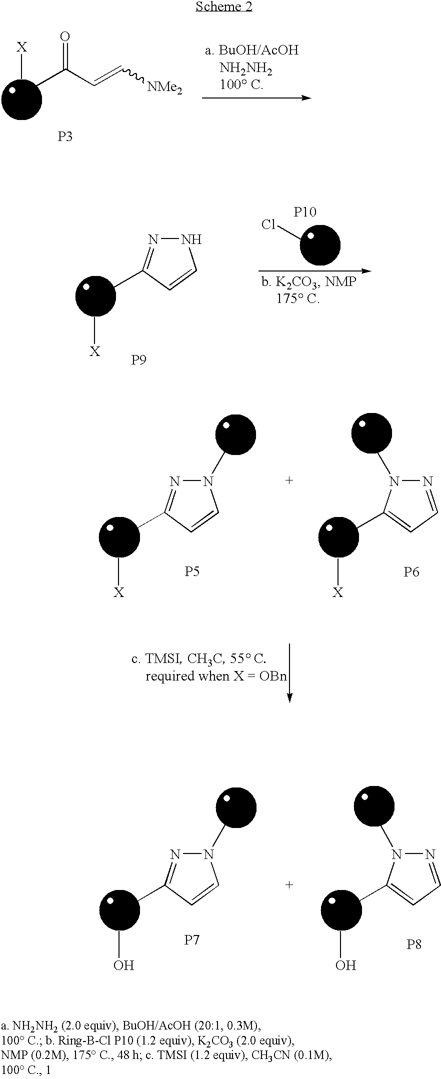

In a typical procedure the same masked ketoaldehyde intermediate P3 is reacted with hydrazine to produce monosubstituted pyrazole P9. Deprotonation of P9 with potassium carbonate in N-methyl pyrolidinone with heating at high temperatures in the presence of chloro-pyridines P10 as the corresponding electrophiles, resulted in the formation of 1 ,3-disubstituted pyrazole P5 as the major products. Small amounts of the 1,2-disubstituted pyrazoles P6 were also obtained, albeit in very low yields (5-10%). Deprotection of the benzyl ethers was accomplished as described previously.

Scheme 3 shows a general procedure to prepare the 1,2- and 1,3-disubstituted pyrazoles from the corresponding diketones.

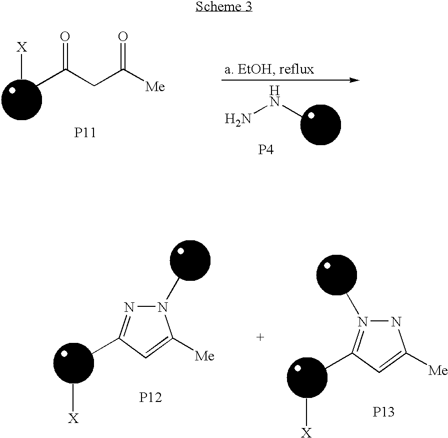

Specifically, diketones P11, obtained from commercial sources or synthesized according to known literature procedures, were reacted with arylhydrazines P4 in refluxing ethanol to furnish pyrazoles P12 and P13.

Scheme 4 shows a general procedure to prepare pyrazole-3-carboxylic acid derivatives from the corresponding diketoesters.

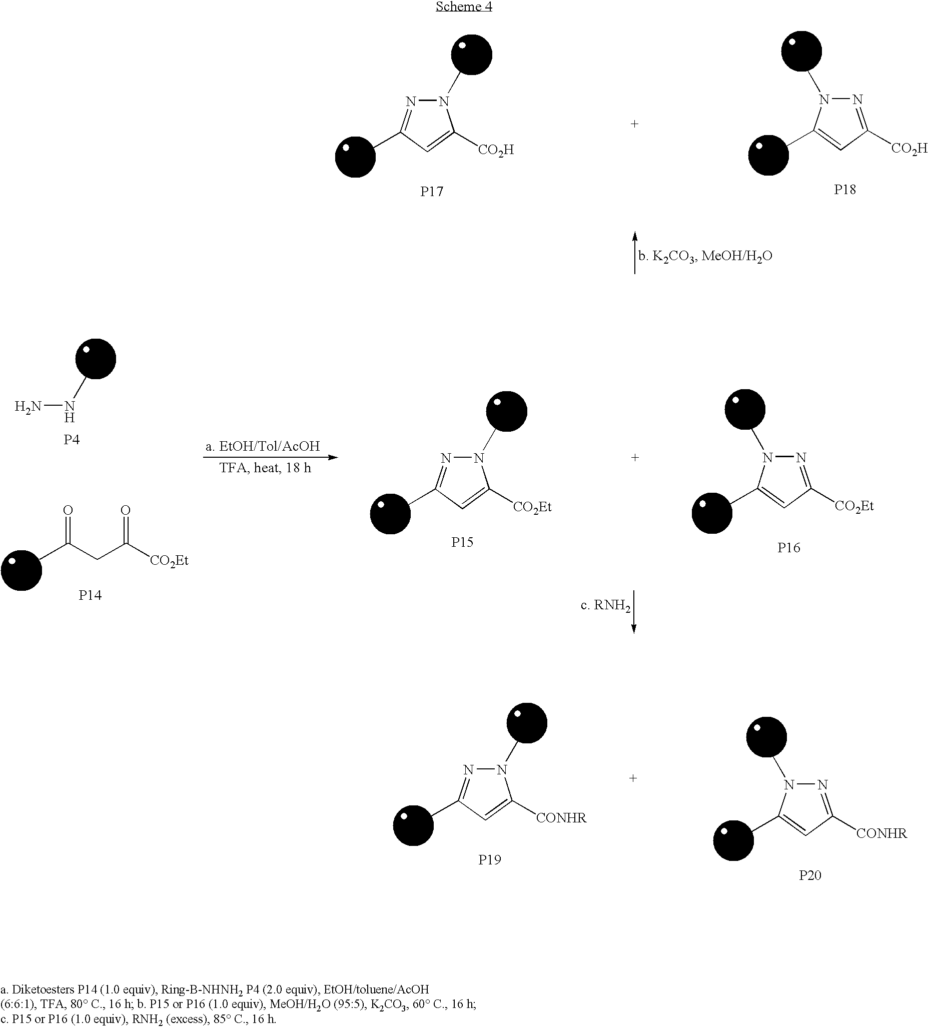

Specifically, diketoesters P14 were reacted with hydrazines P4 to produce a chromatographically separable mixture of pyrazoles P15 and P16. Hydrolysis of the ester functionality firnished carboxylic acids P17 and P18, while treatment with a variety of amines furnished the corresponding amides P19 and P20 in good yields.

### EXAMPLE 1

Synthesis of Compound 84: 4-Methoxy-2-(1-pyridin-2-yl-1H-pyrazol-3-yl)-phenol and Compound 85: 4-Methoxy-2-(2-pyridin-2-yl-2H-pvrazol-3-yl)-phenol.

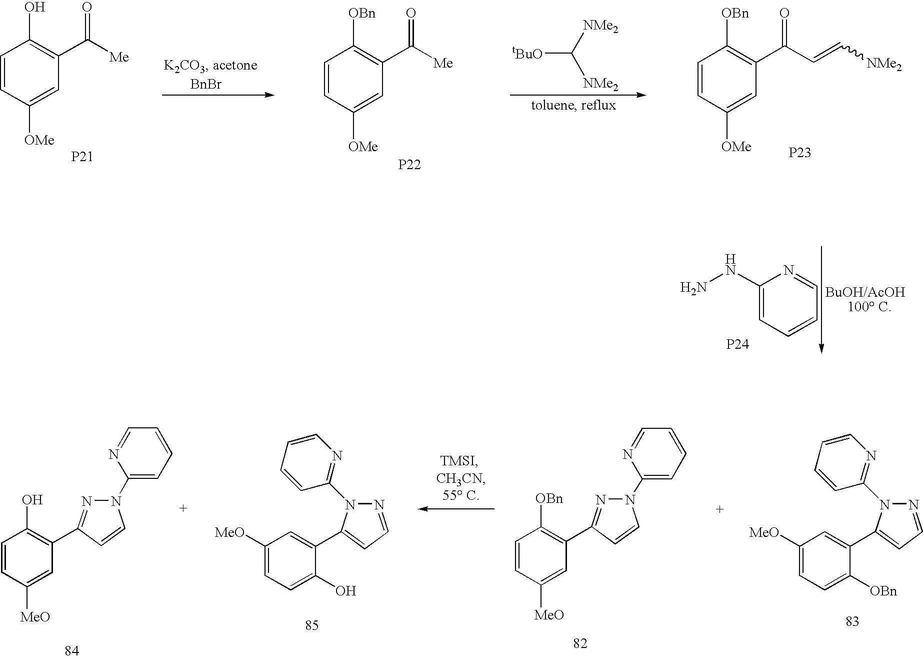

Step 1: Preparation of 1-(2-Benzyloxy-5-methoxy-phenyl)-ethanone P22.

To a solution of phenol P21 (lg, 6.02 mmoles) in acetone there was added solid K₂CO₃ (2.0 equiv, 1.66 g, 12.04 mmoles) followed by the dropwise addition of benzylbromide (1.5 equiv, 1.07 mL, 9.03 mmoles). A condenser was attached and the reaction mixture was refluxed for 16 h under N₂. After cooling to ambient temperature, the solvent was removed under reduced pressure and the solid residue was partitioned between water and ethyl acetate. The organic phase was separated, dried with magnesium sulfate and purified by column chromatography to produce benzyl ether P22 in 97% yield; [M+H]⁺256.8; R_{f}: 0.47 (15% EtOAc/Hex); ¹H (400 MHz, CDCl₃) δ 7.44-7.32 (m, 5H), 7.30 (d, J=3.2 Hz, 1H), 7.00 (dd, J=8.80, 2.80 Hz, 1H), 6.96 (d, J=8.80 Hz, 1H), 5.13 (s, 2H), 3.81 (s, 3H), 2.62 (s, 3H).

Step 2: Preparation of 1-(2-Benzyloxy-5-methoxy-phenyl)-3-dimethylamino-propenone (P23).

To a solution of arylmethylketone P22 (1.5 g, 5.85 mmoles) in toluene (20 mL, 0.3M) was added tert-butoxybis-(dimethylamino)methane (1.1 equiv, 6.44 mmoles) and the reaction mixture was refluxed under N₂ for 5 h. Evaporation of the solvent under reduced pressure furmished P23 in quantitative yield. The product of the reaction was used for the next step without further purification; ¹H (400 MHz, CDCl₃) δ 7.60 (bs, 1H), 7.46-7.26 (m, 4H), 7.21 (bs, 1H), 6.91 (d, J=8.80 Hz, 1H), 6.87 (dd, J=8.80, 3.2 Hz, 1H), 5.75 (bd, J=15.6 Hz 1H), 5.16 (s, 2H), 3.80 (s, 3H), 3.10 (bs, 3H), 2.70 (bs, 3H).

Step 3: Preparation of Compound 82: 2-[3-(2-Benzvloxy-5-methoxy-phenvl)-pvrazol-1-yl]-pyridine and Compound 83: 2-[5-(2-Benzvloxy-5-methoxy-phenyl)-pvrazol-1-yl]-pyridine.

A mixture of masked ketoaldehyde P23 (300 mg, 0.963 mmoles) and 2-pyridyl hydrazine P24 (2.0 equiv, 1.93 mmoles, 210 mg) was dissolved in a butanol/acetic acid solvent system (20:1, 5 mL) and was heated to 100° C. for 5 h. After that time the reaction mixture was cooled to ambient temperature, the solvent was evaporated under reduced pressure and the crude product was purified by HPLC producing 82 (35%) and 83 (30%). For 82: [M+H)⁺358.2; Rf 0.35 (25% EtOAc/hexanes); ¹H (400 MHz, CDCl₃) δ 8.51-8.48 (m, 2H), 8.12 (d, J=8.0 Hz, 1H), 7.89 (dd, J=7.2, 2.0 Hz, 1H), 7.70 (d, J=3.2 Hz, 1H), 7.48-4.44 (m, 2H), 7.42-7.32 (m, 3H), 7.27-7.22 (m, 1H), 7.08 (d, J=2.8 Hz, 1H), 7.00 (d, J=9.2 Hz, 1H), 6.89 (dd, J=8.8, 3.2 Hz, 1H), 5.13 (s, 2H), 3.88 (s, 3H); for 83: [M+H]⁺358.1; ¹H (400 MHz, CDCl₃) δ 8.37 (δ , J=5.2 Hz, 1H), 7.86 (d, J=2.0 Hz, 1H), 7.63 (dd, J=7.2, 1.6 Hz, 1H), 7.26-7.20 (m, 5H), 6.96-6.93 (m, 3H), 6.86 (dd, J=8.8, 2.8 Hz, 1H), 6.77 (d, J=8.8 Hz, 1H), 6.53 (d, J=1.6 Hz, 1H), 4.62 (s, 2H), 3.77 (s, 3H).

Step 4: Preparation of Compound 84: 4-Methoxy-2-(1-pyridin-2-yl-1H-pyrazol-3-yl)-phenol and Compound 85: 4-Methoxy-2-(2-pyridin-2-yl-2H-pyrazol-3-yl)-phenol.

Benzyl ether 82 (100 mg, 0.28 mmoles) was dissolved in acetonitrile (0.1M, 2.8 mL) and heated to 50° C. Trimethylsilyl iodide (1.2 equiv, 0.336 mmoles, 0.05 mL) was then added dropwise and the reaction mixture was stirred for 1 h at the same temperature. After cooling to ambient temperature, the mixture was extracted once with water and the organic phase, after isolation, was partitioned between a saturated sodium thiosulfate solution and ethyl acetate. The aqueous layer was back extracted with ethyl acetate (×2) and the combined organic extracts were dried with MgSO₄ and concentrated. Chromatographic purification furnished the desired product 84 in 60% yield. Rf 0.032 (25% EtOAc/hexanes); ¹H (400 MHz, CDCl₃) δ 10.22 (bs, 1H), 8.64 (d, J=2.8 Hz, 1H), 8.45-8.41 (m, 1H), 7.90-7.82 (m, 2H), 7.24-7.19 (m, 1H), 7.16 (d, J=2.8 Hz, 1H), 7.00 (d, J=8.8 Hz, 1H), 6.88 (dd, J=8.8, 2.8 Hz, 1H), 6.84 (d, J=2.4 Hz, 1H), 3.84 (s, 3H).

Following the same procedure, 85 was obtained in 52% yield. Rf 0.13 (50% EtOAc/hexanes); ¹H (400 MHz, CDCl₃) δ 8.33 (d, J=4.0 Hz, 1H), 7.90 (dd, J=9.6, 8.0 Hz, 1H), 7.83 (d, J=9.2 Hz, 1H), 7.88 (d, J=1.2 Hz, 1H), 7.29 (dd, J=8.4, 5.2 Hz, 1H), 7.04 (d, J=8.8 Hz, 1H), 6.91 (dd, J=8.8, 3.2 Hz, 1H), 6.69 (d, J=3.2 Hz, 1H), 6.43 (d, J=1.6 Hz, 1H), 3.74 (s, 3H).

The following compounds were made according to the procedure of Example 1—Step 1:

1-(2-Benzyloxy-4-methoxy-phenyl)-ethanone (P25): [M+H]⁺257.2:

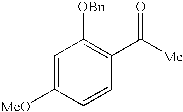

1-(2-Benzyloxy-6-methoxy-phenyl)-ethanone (P26): [M+H]⁺ 257.2:

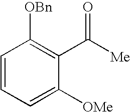

1-(2-Benzyloxy-5-bromo-phenyl)-ethanone (P27): [M+H]⁺ 305.2:

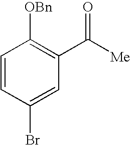

1-(2-Benzyloxy-4,5-dimethoxy-phenyl)-ethanone (P28): [M+H]⁺ 287.3:

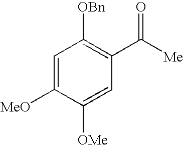

1-(2-Benzyloxy-3,4-dimethoxy-phenyl)-ethanone (P29): [M+H]⁺ 287.3:

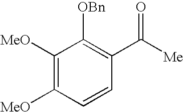

1-(2-Benzyloxy-5-chloro-4-methyl-3-nitro-phenyl)-ethanone (P30): [M+H]⁺ 320.4:

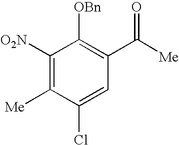

1-(2-Benzyloxy-4-methyl-phenyl)-ethanone (P31): [M+H]⁺ 241.3:

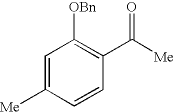

1-(2-Benzyloxy-5-methyl-phenyl)-ethanone (P32): [M+H]⁺ 241.2:

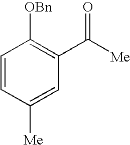

1-(2-Benzyloxy-4,5-dimethyl-phenyl)-ethanone (P33): [M+H]⁺ 256.3:

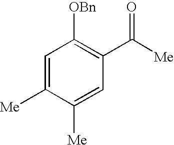

1-(2-Benzyloxy-4-fluoro-phenyl)-ethanone (P34): [M+H]⁺ 245.2:

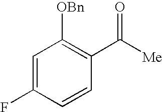

1-(2-Benzyloxy-5-fluoro-phenyl)-ethanone (P35): [M+H]⁺ 245.2:

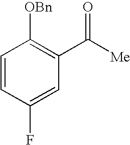

1-(2-Benzyloxy-5-chloro-phenyl)-ethanone (P36): [M+H]⁺ 261.2:

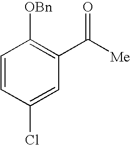

1-(2-Benzyloxy-5-chloro-4-methyl-phenyl)-ethanone (P37): [M+H]⁺ 275.1:

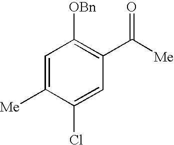

1-(2-Benzyloxy-5-methyl-3-nitro-phenyl)-ethanone (P38): [M+H]⁺ 286.1:

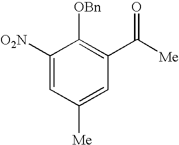

1-(2-Benzyloxy-3-bromo-5-chloro-phenyl)-ethanone (P39): [M+H]⁺ 341.1:

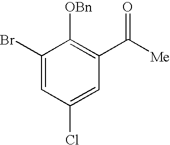

The following compounds were made according to the procedure of Example 1—Step 2:

3-Dimethylamino-1-(2-methoxy-phenyl)-propenone (P40): ¹H (400 MHz, CDCl₃) δ 7.53 (bs, 1H), 7.46 (bd, J=8.8,Hz, 1H), 7.33 (dd, J=8.8, 7.2 Hz, 1H), 6.96 (dd, J=8.8, 8.8 Hz, 1H), 6.93 (d, J=8.8 Hz, 1H), 5.55 (d, J=15.6 Hz, 1H), 3.86 (s, 1H), 3.06 (bs, 3H), 2.90 (bs, 3H):

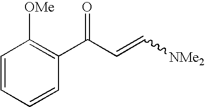

1-(2-Benzyloxy-naphthalen-1-yl)-3-dimethylamino-propenone (P41): ¹H (400 MHz, CDCl₃) δ 7.83 (bd, J=8.0 Hz, 1H), 7.78-7.72 (m, 2H), 7.45-7.39 (m, 3H), 7.36-7.30 (m, 3H), 7.29-7.23 (m, 3H), 5.51 (bd, J=14.8 Hz, 1H), 5.24 (s, 2H), 2.92 (bs, 3H), 2.84 (bs, 3H):

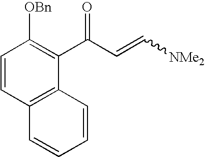

3-Dimethylamino-1-(2-fluoro-4-methoxy-phenyl)-propenone (P42): 1H (400 MHz, CDCl₃) δ 7.82 (dd, J=8.8, 8.8 Hz, 1H), 7.78 (bd, J=13.2 Hz, 1H), 6.72 (dd, J=8.4, 2.0 Hz, 1H), 6.58 (dd, J=12.8, 2.4 Hz, 1H), 5.68 (dd, J=12.4, 2.0 Hz, 1H), 3.84 (s, 3H), 3.14 (bs, 3H), 2.91 (bs, 3H):

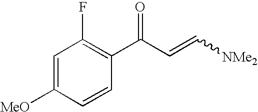

The following masked ketoaldehydes were synthesized and used in the following step without further characterization.

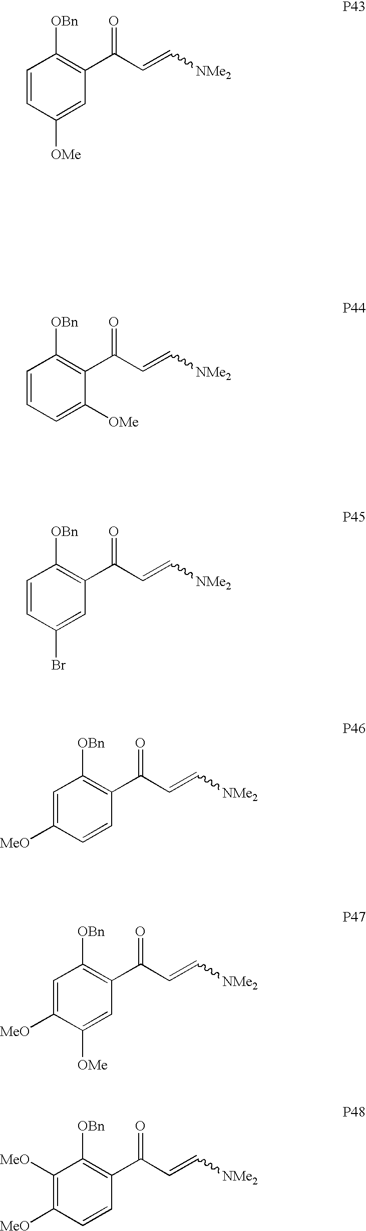

The following compounds were made according to the procedure of Example 1—Step 3:

1-(2-Fluoro-phenyl)-3-(2-methoxy-phenyl)-1H-pyrazole (33): [M+H]⁺ 268.7; ¹H (400 MHz, CDCl₃) δ 7.80 (d, J=1.6 Hz, 1H), 7.32-7.23 (m, 4H), 7.11-7.04 (m, 2H), 6.94 (dd, J=8.0, 7.6 Hz, 1H), 6.78 (d, J=8.0 Hz, 1H), 6.52 (d, J=1.6 Hz, 1H), 3.50 )s, 3H):

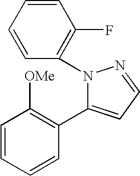

2-[3-(2-Methoxy-phenyl)-pyrazol-1-yl]-3-nitro-pyridine (36): ¹H (400 MHz, CDCl₃) δ 8.49 (dd, J=4.8, 1.2 Hz, 1H), 8.20 (dd, J=8.0, 1.6 Hz, 1H), 7.78 (d, J=1.6 Hz, 1H), 7.41-7.32 (m, 3H), 7.01 (ddd, J=7.6, 7.6, 1.2 Hz, 1H), 6.77 (d, J=7.6 Hz, 1H), 6.54 (d, J=1.6 Hz, 1H), 3.41 (s, 3H):

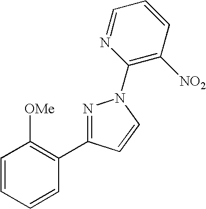

3-(2-Methoxy-phenyl)-1-(2-nitro-phenyl)-1H-pyrazole (31): [M+H]⁺ 296.3; ¹H (400 MHz, CDCl₃) δ 7.94 (dd, J=7.6, 1.6 Hz, 1H), 7.86 (d, J=8.0 Hz, 1H), 7.74 (d, J=2.4 Hz, 1H), 7.69-7.63 (m, 2H), 7.51-7.44 (m, 1H), 7.32 (ddd, J=7.6, 7.2, 2.0 Hz, 1H), 7.05 (d, J=2.4 Hz, 1H), 7.01 (ddd, J=7.6, 7.2, 1.2 Hz, 1H), 6.98 (d, J=8.4 Hz, 1H), 3.93 (s, 3H):

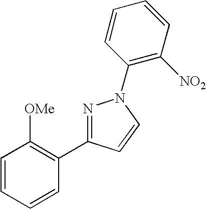

5-(2-Methoxy-phenyl)-1-(2-nitro-phenyl)-1H-pyrazole (32): ¹H (400 MHz, CDCl₃) δ 7.82 (dd, J=8.0, 1.6 Hz, 1H), 7.78 (d, J=2.0 Hz, 1H), 7.53 (dd, J=8.4, 7.6 Hz, 1H), 7.44-7.36 (m, 2H), 7.32 (dd, J=8.0, 7.2 Hz, 1H), 7.23 (dd, J=7.2, 1.2 Hz, 1H), 6.94 (dd, J=7.6, 1.2 Hz, 1H), 6.78 (d, J=8.0 Hz, 1H), 6.51 (d, J=1.6 Hz, 1H), 3.43 (s, 3H):

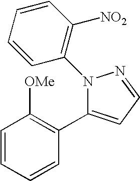

2-[5-(2-Methoxy-phenyl)-pyrazol-1-yl]-3-nitro-pyridine (37): [M+H]⁺ 297.3; ¹H (400 MHz, CDCl₃) δ 8.55 (d, J=4.8 Hz, 1H), 8.41 (d, J=3.2 Hz, 1H), 7.97 (d, J=8.0 Hz, 1H), 7.92 (d, J=7.6 Hz, 1H), 7.35-7.26 (m, 1H), 7.08 (d, J=3.2 Hz, 1H), 7.03 (dd, J=8.0, 7.2 Hz, 1H), 6.97 (d, J=4.8 Hz, 1H), 3.93 (s. 3H):

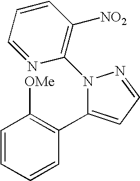

2-[3-(2-Methoxy-phenyl)-pyrazol-1-yl]-pyridine (39): ¹H (400 MHz, CDCl₃) δ 8.23 (d, J=4.8 Hz, 1H), 7.75 ((d, J=1.2 Hz, 1H), 7.71 ((ddd, J=7.6, 7.2, 2.0 Hz, 1H), 7.54 ((d, J=8.0 Hz, 1H), 7.40-7.31 (m, 2H), 7.12 ((ddd, J=7.2, 4.8, 1.2 Hz, 1H), 7.01 (ddd, J=8.0, 8.0, 1.2 Hz, 1H), 6.77 (d, J=4.8 Hz, 1H), 6.46 (d, J=1.6 Hz, 1H), 3.36 (s, 3H):

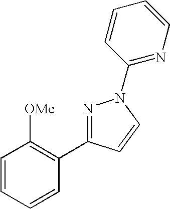

2-[5-(2-Methoxy-phenyl)-pyrazol-1-yl]-pyridine (40): ¹H (400 MHz, CDCl₃) δ 8.58 (d, J=2.4 Hz, 1H),8.41 (d,J=4.0Hz, 1H),8.11 (d,J=8.8 Hz, 1H),7.81 (ddd,J=7.6,7.2, 1.6 Hz, 1H), 7.34 (ddd, J=7.6, 7.6, 2.0 Hz, 1H), 7.16 (d, J=7.2, 4.8, 0.8 Hz, 1H), 7.06 (ddd, J=8.0, 7.2, 1.2 Hz, 1H), 7.03 (d, J=2.8 Hz, 1H), 7.01 (d, J=8.4 Hz, 1H), 3.96 (s, 3H):

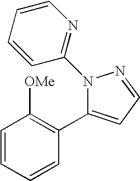

2-[3-(2-Methoxy-phenyl)-pyrazol-1-yl]-3-trifluoromethyl-pyridine (38): ¹H (400 MHz, CDCl₃) δ 8.64 (dd, J=4.8, 1.6 Hz, 1H), 8.30 (d, J=2.4 Hz, 1H), 8.20 (dd, J=8.0, 1.6 Hz, 1H), 8.14 (dd, J=7.6, 1.6 Hz, 1H), 7.37 (dd, J=8.4, 4.8 Hz, 1H), 7.35-7.30 (m, 1H), 7.10 (d, J=2.8 Hz, 1H), 7.05 (ddd, J=8.4, 8.0, 1.2 Hz, 1H), 7.00 (d, J=8.0 Hz, 1H), 3.95 (s, 3H):

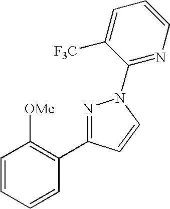

2-[3-(2-Fluoro-4-methoxy-phenyl)-pyrazol-1-yl]-3-nitro-pyridine (45): [M+H]⁺ 315.2; ¹H (400 MHz, CDCl₃) δ 8.57 (dd, J=4.8, 1.6 Hz, 1H), 8.43 (d, J=2.4 Hz, 1H), 8.00 (dd, J=8.0, 1.6 Hz, 1H), 7.87 (dd, J=8.8, 8.8 Hz, 1H), 7.33 (dd, J=8.0, 7.6 Hz, 1H), 6.90 (dd, J=3.6, 2.4 Hz, 1H), 6.77 (dd, J=8.8, 2.4 Hz, 1H), 6.66 (dd, J=9.2, 2.8 Hz, 1H), 3.83 (s, 3H):

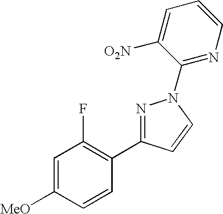

2-[5-(2-Fluoro-4-methoxy-phenyl)-pyrazol-1-yl]-3-nitro-pyridine (46): ¹H (400 MHz, CDCl₃) δ 8.52 (dd, J=8.0, 1.2 Hz, 1H), 8.23 (dd, J=8.0, 1.6 Hz, 1H), 7.78 (d, J=1.6 Hz, 1H), 7.44 (dd, J=8.0, 5.2 Hz, 1H), 7.24 (dd, J=8.8, 8.4 Hz, 1H), 6.69 (dd, J=8.4, 2.8 Hz, 1H), 6.58-6.54 (m, 2H), 3.81 (s, 3H):

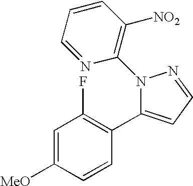

3-Chloro-2-[3-(2-fluoro-4-methoxy-phenyl)-pyrazol-1-yl]-5-trifluoromethyl-pyridine (47): [M+H]⁺ 372.2; ¹H (400 MHz, CDCl₃) δ 8.67 (s, 1H), 8.36 (d, J=2.8 Hz, 1H), 8.14 (s, 1H), 8.05 (dd, J=8.8, 8.4 Hz, 1H), 6.93 (dd, J=8.0, 7.6 Hz, 1H), 6.79 (dd, J=8.8, 2.8 Hz, 1H), 6.71 (dd, J=12.8, 2.0 Hz, 1H), 3.83 (s, 3H):

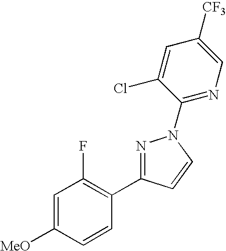

3-Chloro-2-[5-(2-fluoro-4-methoxy-phenyl)-pyrazol-1-yl]-5-trifluoromethyl-pyridine (48): ¹H (400 MHz, CDCl₃) δ 8.59 (s, 1H), 8.08 (d, J=2.4 Hz, 1H), 7.86 (d, J=1.6 Hz, 1H), 7.13 (dd, J=8.8, 8.4 Hz, 1H), 6.65 (dd, J=8.4, 2.8 Hz, 1H), 6.59 (s, 1H), 6.56 (dd, J=12.0, 2.4 Hz, 1H), 3.81 (s, 3H):

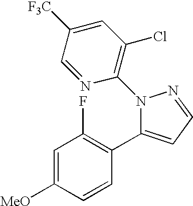

3,5-Dichloro-4-[3-(2-fluoro-4-methoxy-phenyl)-pyrazol-1-yl]-pyridine (49): [M+H]⁺ 338.2; ¹H (400 MHz, CDCl₃) δ 8.67 (s, 1H), 7.95 (dd, J=8.8, 8.4 Hz, 1H), 7.66 (d, J=2.4 Hz, 1H), 6.93 (dd, J=3.6, 2.8 Hz, 1H), 6.80-6.65 (m, 3H), 3.82 (s, 3H).

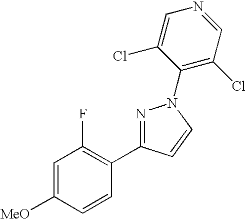

3,5-Dichloro-4-[5-(2-fluoro-4-methoxy-phenyl)-pyrazol-1-yl]-pyridine (50): ¹H (400 MHz, CDCl₃) δ 8.57 (s, 1H), 7.89 (d, J=1.6 Hz, 1H), 7.00 (dd, J=8.4, 8.0 Hz, 1H), 6.61-6.59 (m, 4H), 3.82 (s, 3H).

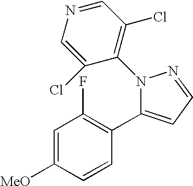

2-[3-(2-Fluoro-4-methoxy-phenyl)-pyrazol-1-yl]-3-trifluoromethyl-pyridine (51): [M+H]+ 338.2; ¹H (400 MHz, CDCl₃) δ 8.65 (d, J=4.8 Hz, 1H), 8.30 (d, J=2.4 Hz, 1H), 8.21 (dd, J=7.6, 1.6 Hz, 1H), 8.06 (dd,J=8.8, 8.8 Hz, 1H), 7.40 (dd, J=8.8, 8.4 Hz, 1H), 6.91 (dd,J=4.0, 2.4 Hz, 1H), 6.80 (dd, J=8.0, 2.4 Hz, 1H), 6.69 (dd, J=8.8, 2.0 Hz, 1H), 3.82 (s, 3H):

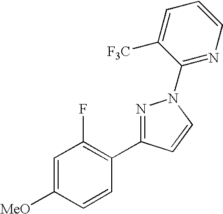

2-[5-(2-Fluoro-4-methoxy-phenyl)-pyrazol-1-yl]-3-trifluoromethyl-pyridine (52): ¹H (400 MHz, CDCl₃) δ 8.55 (dd,J=4.8, 1.2 Hz, 1H), 8.16 (d, J=8.0, 1.6 Hz, 1H), 7.79 (d,J=1.6 Hz, 1H), 7.46 (dd, J=8.0, 4.8 Hz, 1H), 7.15 (dd,J=8.8, 8.4 Hz, 1H), 6.62 (dd, J=8.8, 2.4 Hz, 1H), 6.57 (s, 1H), 6.52 (dd, J=12.0, 2.8 Hz, 1H), 3.79 (s, 3H):

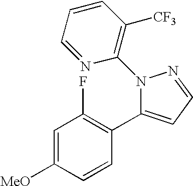

2-[3-(2-Fluoro-4-methoxy-phenyl)-pyrazol-1-yl]-pyridine (53): [M+H]⁺ 270.1; 1H (400 MHz, CDCl3) δ 18.57 (d, J=2.8 Hz, 1H), 8.42 (d, J=5.2 Hz, 1H), 8.09-8.03 (m, 2H), 7.83 (ddd, J=8.0, 7.2, 2.0 Hz, 1H), 7.18 (ddd, J=6.0, 4.8, 0.8 Hz, 1H), 6.86 (dd, J=4.0, 2.8 Hz, 1H), 6.80 (dd, J=8.8, 2.4 Hz, 1H), 6.70 (dd, J=12.8, 2.0 Hz, 1H), 3.82 (s, 3H):

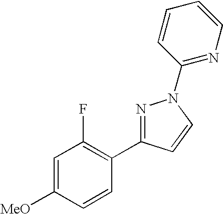

2-[5-(2-Fluoro-4-methoxy-phenyl)-pyrazol-1-yl]-pyridine (54): ¹H (400 MHz, CDCl₃) δ 8.25 (d, J=4.4 Hz, 1H), 7.79-7.74 (m, 2H), 7.65 (d, J=8.0 Hz, 1H), 7.24 (dd, J=8.8, 8.4 Hz, 1H), 7.17 (dd, J=7.2,4.8 Hz, 1H), 6.70 (dd, J=8.8, 2.8 Hz, 1H), 6.56 (dd, J=11.6, 2.4 Hz, 1H), 6.48 (d, J=2.0 Hz, 1H), 3.82, (s, 3H):

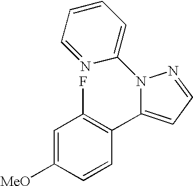

2-[3-(2-Benzyloxy-5-methoxy-phenyl)-pyrazol-1-yl]-3-trifluoromethyl-pyridine (80): [M+H]⁺ 426.2; ¹H (400 MHz, CDCl₃) δ 8.65 (d, J=4.8 Hz, 1H), 8.25 (d, J=2.4 Hz, 1H), 8.21 (d, J=8.0 Hz, 1H), 7.79 (d, J=2.8 Hz, 1H), 7.47-7.33 (m, 6H), 7.11 (dd,J=2.8, 1.2 Hz, 1H), 6.98 (d, J=8.8 Hz, 1H), 6.87 (dd, J=8.8, 3.2 Hz, 1H), 5.13 (s, 2H), 3.86 (s, 3H):

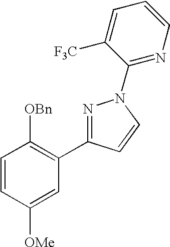

2-[3-(2-Benzyloxy-5-methoxy-phenyl)-pyrazol-1-yl]-3-nitro-pyridine (P49): ¹H (400 MHz, CDCl₃) δ 8.55 (d, J=4.8 Hz, 1H), 8.37 (d, J=2.8 Hz, 1H), 7.96 (d, J=7.6 Hz, 1H), 7.57 (d, J=3.2 Hz, 1H), 7.46-7.28 (m, 6H), 7.09 (d, J=2.8 Hz, 1H), 6.95 (d, J=9.2 Hz, 1H), 6.86 (dd, J=8.8, 3.2 Hz, 1H), 5.11 (s, 2H), 3.87 (s, 3H):

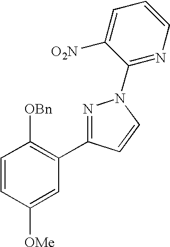

2-[3-(2-Benzyloxy-naphthalen-1-yl)-pyrazol-1-yl]-pyridine (87): ¹H (400 MHz, CDCl₃) δ 8.72 (d, J=2.4 Hz, 1H), 8.45 (d, J=4.8 Hz, 1H), 8.08 (d, J=8.4 Hz, 1H), 8.03 (d, J=8.4 Hz, 1H), 7.86 (d, J=9.2 Hz, 1H), 7.82-7.76 (m, 3H), 7.45-7.16 (m, 8H), 6.70 (d, J=2.8 Hz, 1H), 5.21 (s, 2H):

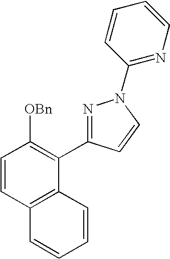

2-[3-(2-Benzyloxy-5-bromo-phenyl)-pyrazol-1-yl]-3-trifluoromethyl-pyridine (108): [M+H]⁺ 475.2:

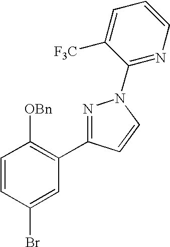

2-[3-(2-Benzyloxy-naphthalen-1-yl)-pyrazol-1-yl]-3-nitro-pyridine (P50): [M+H]⁺ 423.3:

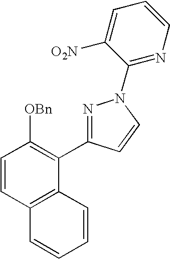

2-[3-(2-Benzyloxy-4-methoxy-phenyl)-pyrazol-1-yl]-3-methyl-pyridine (92): [M+H]⁺ 372.3:

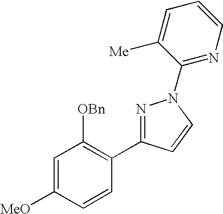

2-[3-(2-Benzyloxy-6-methoxy-phenyl)-pyrazol-1-yl]-pyridine 96: [M+H]⁺ 358.3:

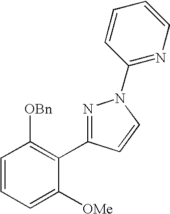

2-[3-(2-Benzyloxy-5-bromo-phenyl)-pyrazol-1-yl]-pyridine 98: [M+H]⁺ 408.3:

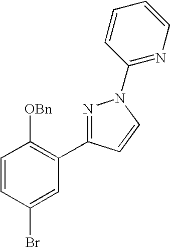

2-[3-(2-Benzyloxy-4,5-dimethoxy-phenyl)-pyrazol-1-yl]-pyridine (121): [M+H]⁺ 388.4:

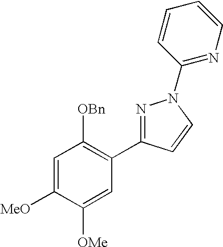

The following compounds were made according to the procedure of Example 1—Step 4:

4-Methoxy-2-[l-(3-methyl-pyridin-2-yl)-1H-pyrazol-3-yl]-phenol (93): [M+H]⁺ 282.1; ¹H (400 MHz, DMSO-d6) δ 9.88 (bs, 1H), 8.45 (d, J=2.4 Hz, 1H), 8.37 (d, J=4.8 Hz, 1H), 7.88 (d, J=8.4 Hz, 1H), 7.40 (d, J=2.8 Hz, 1H), 7.37 (dd, J=7.6, 7.2 Hz, 1H), 7.17 (d, J=2.4 Hz, 1H), 6.87 (d, J=8.8 Hz, 1H), 6.82 (dd, J=8.8, 2.8 Hz, 1H), 3.74 (s, 3H), 2.52 (s, 3H):

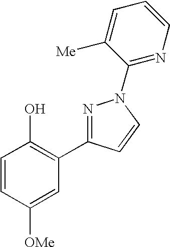

5-Methoxy-2-(1-pyridin-2-yl-1H-pyrazol-3-yl)-phenol (88): [M+H]⁺ 268.3; ¹H (400 MHz, CDCl₃) δ 8.69 (d, J=2.8 Hz, 1H), 8.44 (d, J=5.6 Hz, 1H), 7.89-7.87 (m, 2H), 7.52 (d, J=9.2 Hz, 1H), 7.26-7.21 (m, 1H), 6.78 (d, J=2.4 Hz, 1H), 6.60 (d, J=2.0 Hz, 1H), 6.54 (dd, J=8.8, 2.4 Hz, 1H), 3.85 (s, 3H):

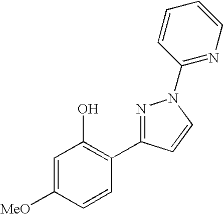

4-Bromo-2-(1-pyridin-2-yl-1H-pyrazol-3-yl)-phenol (97): [M+H]⁺ 316.1; ¹H (400 MHz, DMSO-d6) δ 10.41 (s, 1H), 8.45 (d, J=2.4 Hz, 1H), 8.67 (d, J=2.8 Hz, 1H), 8.47 (d, J=4.8 Hz, 1H), 8.07 (d, J=2.8 Hz, 1H), 8.01-7.99 (m, 1H), 7.38-7.34 (m, 2H), 7.19 (d, J=2.8 Hz, 1H), 6.93 (d, J=8.4 Hz, 1H):

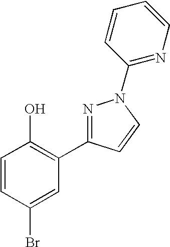

4-Bromo-2-[1-(3-trifluoromethyl-pyridin-2-yl)-1H-pyrazol-3-yl]-phenol (100): ¹H (400 MHz, CDCl₃) δ 10.33 (s, 1H), 8.67 (d, J=4.4 Hz, 1H), 8.44 (d, J=3.2 Hz, 1H), 8.23 (d, J=8.4 Hz, 1H), 7.72 (d,J=2.8 Hz, 1H), 7.45 (dd,J=8.0, 4.8 Hz, 1H), 7.34 (dd,J=8.8, 8.4 Hz, 1H), 6.94 (d, J=8.4 Hz, 1H), 6.88 (d, J=2.8 Hz, 1H):

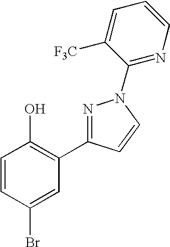

5-Methoxy-2-[1-(3-methyl-pyridin-2-yl)-1H-pyrazol-3-yl]-phenol (91): [M+H]⁺ 282.2; ¹H (400 MHz, DMSO-d6) δ 10.46 (s, 1H), 8.43 (d, J=2.8 Hz, 1H), 8.36 (dd, J=4.4, 1.6 Hz, 1H), 7.87 (d, J=7.2 Hz, 1H), 7.74 (d, J=7.2 Hz, 1H), 7.36 (dd, J=7.2, 4.4 Hz, 1H), 7.03 (d, J=2.8 Hz, 1H), 6.52 (dd, J=9.2, 2.8 Hz, 1H), 6.51 (s, 1H), 3.75 (s, 3H), 3.52 (s, 3H):

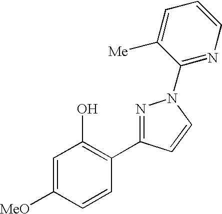

4-Bromo-2-[1-(3-methyl-pyridin-2-yl)-1H-pyrazol-3-yl]-phenol (99): [M+H]⁺ 332.0; ¹H (400 MHz, DMSO-d6) δ 10.48 (s, 1H), 8.43 (d, J=2.8 Hz, 1H), 8.37 (d, J=4.4 Hz, 1H), 8.00 (d, J=2.8 Hz, 1H), 7.90 (d, J=5.2 Hz, 1H), 7.39 (dd, J=8.0, 7.6 Hz, 1H), 7.33 (dd, J=8.4, 2.8 Hz, 1H), 7.17 (d, J=2.4 Hz, 1H), 6.92 (d,J=8.8 Hz, 1H), 2.52 (s, 3H):

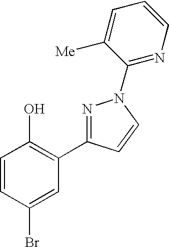

2,3-Dimethoxy-6-(1-pyridin-2-yl-1H-pyrazol-3-yl)-phenol (118): [M+H]⁺ 298.3:

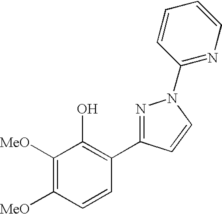

2,3-Dimethoxy-6-[1-(3-methyl-pyridin-2-yl)-1 H-pyrazol-3-yl]-phenol (119): [M+H]⁺ 312.3:

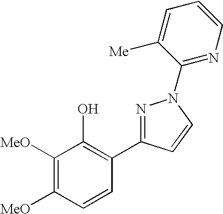

4,5-Dimethoxy-2-(1-pyridin-2-yl-1H-pyrazol-3-yl)-p henol (120): [M+H]⁺ 298.2:

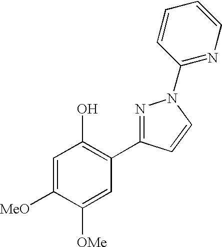

4,5-Dimethoxy-2-[l-(3-methyl-pyridin-2-yl)-1H-pyrazol-3-yl]-phenol (122): [M+H]⁺ 312.2:

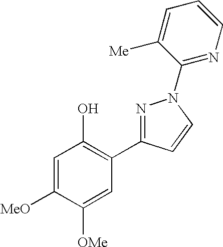

### EXAMPLE 2

Deprotection of methyl ethers with AlBr3 and synthesis of 2-[1-(2-Nitro-phenyl)-1H-pyrazol-3-yl]-phenol (34).

A solution of methyl ether 32 (46 mg, 0.156 mmoles) in CH₂Cl₂ (1 mL) was added dropwise to a mixture of EtSH (3 mL) and AlBr₃ (2.5 equiv, 52 mg, 0.39 mmoles) at ambient temperature. The reaction progress was monitor by TLC to the disappearance of the starting material, after which time the mixture was quenched by the addition of HCl (10 mL, 0.1 M). The organic phase was separated and the aqueous one was extracted three times with CH₂Cl₂. The combined organic extracts were dried with MgSO₄, followed by evaporation of the solvent under reduced pressure. Chromatographic purification firnished the desired phenol 34 in 83% yield. Rf 0.27 (40% EtOAc/hexanes); ¹H (400 MHz, CDCl₃) δ 10.11 (bs, 1H), 7.91 (d, J=8.0 Hz, 1H), 7.76 (d, J=2.4 Hz, 1H), 7.73 (dd, J=8.0, 8.0 Hz, 1H), 7.65 (d, J=7.6 Hz, 1H), 7.60 (dd, J=7.6, 1.6 Hz, 1H), 7.56 (dd, J=8.0, 7.6 Hz, 1H), 7.28-7.23 (m, 1H), 7.04 (d, J=8.0 Hz, 1H), 6.94 (dd, J=7.6, 7.6 Hz, 1H), 6.89 (d, J=2.8 Hz, 1H):

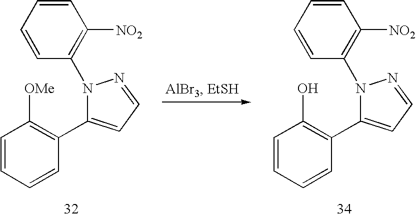

The following compounds were made according to the procedure of Example 2:

2-[1-(3-Nitro-pyridin-2-yl)-1H-pyrazol-3-yl]-phenol (41): ¹H (400 MHz, CDCl₃) δ 9.47 (s, 1H), 8.62 (dd, J=4.8, 1.6 Hz, 1H), 8.54 (d, J=2.8 Hz, 1H), 8.06 (dd, J=8.0, 1.6 Hz, 1H), 7.60 (dd, J=8.0, 1.6 Hz, 1H), 7.40 (dd, J=8.0, 4.8 Hz, 1H), 7.28 (ddd, J=8.4, 7.2, 4.8, 1.6 Hz, 1H), 7.07 (dd, J=8.4, 0.8 Hz, 1H), 6.94 (dd, J=8.4, 6.8, 1.6 Hz, 1H), 6.92 (d, J=3.2 Hz, 1H):

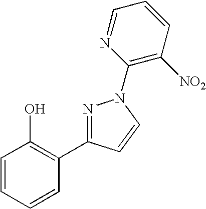

2-[2-(2-Fluoro-phenyl)-2H-pyrazol-3-yl]-phenol (35): ¹H (400 MHz, CDCl₃) δ 7.86 (d, J=2.0 Hz, 1H), 7.42 (ddd, J=8.0, 7.6, 1.6 Hz, 1H), 7.35-7.28 (m, 1H), 7.24-7.14b (m, 2H), 7.05 (ddd, J=10.0, 9.6, 1.6 Hz, 1H), 6.93-6.89 (m, 2H), 6.76 (ddd, J=8.4, 7.6, 1.2 Hz, 1H), 6.61 (d, J=2.0 Hz, 1H), 5.43 (s, 1H):

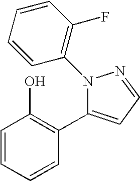

2-[1-(3-Trifluoromethyl-pyridin-2-yl)-1H-pyrazol-3-yl]-phenol (42): Rf: 0.44 (30% EtOAc/hexanes); ¹H (400 MHz, CDCl₃) δ 10.32 (s, 1H), 8.67 (dd, J=4.8, 1.6 Hz, 1H), 8.44 (d, J=2.8 Hz, 1H), 8.23 (dd, J=8.0, 1.6 Hz, 1H), 7.64 (dd, J=7.6, 1.2 Hz, 1H), 7.46-7.42 (m, 1H), 7.30-7.26 (m, 1H), 7.07 (dd, J=8.4, 1.2 Hz, 1H), 6.95 (ddd, J=8.4, 8.0, 1.6Hz, 1H), 6.92 (d, J=2.8 Hz, 1H):

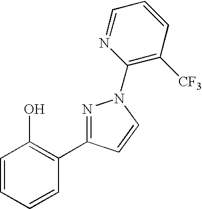

2-(2-Pyridin-2-yl-2H-pyrazol-3-yl)-phenol (44): Rf: 0.57 (30% EtOAc/hexanes); ¹H (400 MHz, CDCl₃) δ 10.68 (s, 1H), 8.64 (d, J=2.8 Hz, 1H), 8.45-8.43 (m, 1H), 7.90-7.83 (m, 2H), 7.64 (dd, J=7.6, 1.6 Hz, 1H), 7.28 (ddd, J=8.4, 6.4, 2.0 Hz, 1H), 7.23 (ddd, J=6.8, 4.4, 2.4 Hz, 1H), 7.08 (dd, J=8.0, 0.8 Hz, 1H), 6.96 (ddd, J=8.4, 7.6, 1.2 Hz, 1H), 6.88 (d, J=6.8 Hz, 1H):

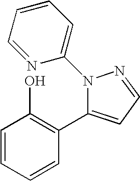

2-(1-Pyridin-2-yl-1H-pyrazol-3-yl)-phenol (43): Rf: 0.11 (30% EtOAc/hexanes); ¹H (400 MHz, CDCl₃) δ 10.21 (bs, 1H), 8.34-8.32 (m, 1H), 7.93-7.86 (m, 1H), 7.82 (d, J=8.0 Hz, 1H), 7.79 (d, J=2.0 Hz, 1H), 7.35 (ddd, J=9.2, 7.2, 2.0 Hz, 1H), 7.29 (ddd, J=7.6, 7.2, 1.2 Hz, 1H), 7.15, 7.11 (m, 2H), 6.93 (ddd, J=7.6, 7.2,1.2 Hz, 1H), 6.41 (d, J=1.6 Hz, 1H):

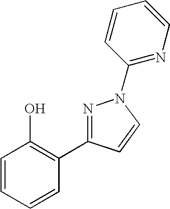

2-[1-(3-Nitro-pyridin-2-yl)-1H-pyrazol-3-yl]-phenol (41): ¹H (400 MHz, CDCl₃) δ 9.46 (s, 1H), 8.62 (dd, J=4.4, 1.6 Hz, 1H), 8.54 (d, J=2.8 Hz, 1H), 8.06 (dd, J=8.0, 1.2 Hz, 1H), 7.60 (dd, J=8.0, 1.6 Hz, 1H), 7.40 (dd, J=8.4, 4.8 Hz, 1H), 7.30 (ddd, J=8.4, 7.6, 1.6 Hz, 1H), 7.07 (d, J=8.4 Hz, 1H), 6.95 (d, J=8.4 Hz, 1H), 6.92 (d, J=2.8 Hz, 1H):

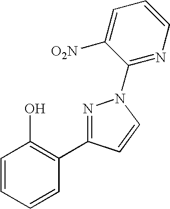

### EXAMPLE 3

Preparation of 4-Methoxy-2-[1-(3-trifluoromethyl-pyridin-2-yl)-1H-pyrazol-3-yl]-phenol (89).

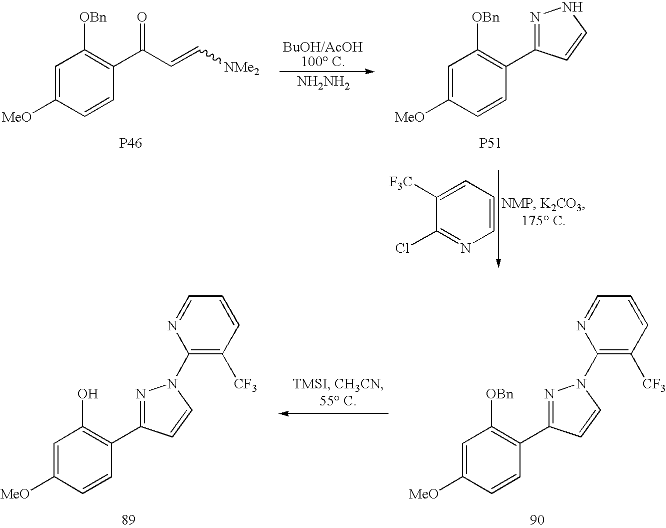

Step 1: Preparation of 3-(2-Benzyloxy-4-methoxy-phenyl)-1H-pyrazole (P51).

The same procedure presented in example 1, step 3, was followed, producing pyrazole P51 in quantitative yield. The product was used for the next step without purification. [M+H]⁺ 281.3; ¹H (400 MHz, DMSO-d6) δ 7.70 (d, J=8.8 Hz, 1H), 7.53 (d, J=1.6 Hz, 1H),7.48-7.45 (m, 2H), 7.40-7.34 (m, 3H), 6.72 (d, J=2.4 Hz, 1H), 6.58 (dd, J=8.8, 2.4 Hz, 1H), 6.56 (d, J=2.4 Hz, 1H), 5.21 (s, 2H), 3.76 (s, 3H).

Step 2: 2-13-(2-Benzyloxy-4-methoxy-phenyl)-pvrazol-1-yl]-3-trifluoromethyl-pyridine (90).

Pyrazole P51 (70 mg, 0.25 mmoles), 2-chloro-3-trifluoromethyl pyridine (1.2 equiv, 0.3 mmoles, 55 mg) and K₂CO₃ (2.0 equiv, 0.5 mmoles, 70 mg) were dissolved in NMP (2 mL) and heated to 175° C. for 48 h. After cooling to ambient temperature, water was added and the reaction mixture was extracted with ethylacetate (5 mL, ×3). The organic extracts were combined, the solvent was evaporated under reduced pressure and the organic residue was purified by HPLC to produce the final 1,3-disubstituted pyrazole 90 in 86% yield. ¹H (400 MHz, CDCl₃) δ 8.60 (dd, J=4.8, 1.6 Hz, 1H), 8.23 (d, J=2.4 Hz, 1H), 8.17 (dd, J=8.0, 2.0 Hz, 1H), 8.14 (d, J=8.4 Hz, 1H), 7.49-7.31 (m, 6H), 7.01 (d, J=2.4 Hz, 1H), 6.64-6.61 (m, 2H), 5.16 (s, 2H), 3.83 (s, 3H); 13C (100 MHz, CDCl₃) ,160.6, 157.1, 151.1, 150.9, 148.9, 137.8 (q, J=5.3 Hz), 136.6, 129.8, 129.5, 128.4, 127.8, 127.4, 124.1, 121.4, 121.1, 114.9, 109.1, 105.6, 99.9, 70.5, 55.4.

Step 3: 5-Methoxy-2-[1-(3-trifluoromethyl-pyridin-2-yl)-1H-pyrazol-3-yl]-phenol (89).

The final deprotection was performed as described before in example 1—step 4, to produce phenol 89 in 77% yield. [M+H]⁺ 336.3; ¹H (400 MHz, CDCl₃) δ 10.46 (bs, 1H), 8.64 (d, J=4.8 Hz, 1H), 8.42 (d, J=2.8 Hz, 1H), 8.21 (d, J=7.6 Hz, 1H), 7.52 (d, J=8.4 Hz, 1H), 7.40 (dd, J=8.4, 4.8 Hz, 1H), 6.82 (d, J=2.8 Hz, 1H), 6.61 (d, J=2.4 Hz, 1H), 6.54 (dd, J=8.4, 2.4 Hz, 1H), 3.81 (s, 3H); ¹³C (100 MHz, CDCl₃) δ 161.2, 157.3, 154.2, 151.1, 138.0 (q, J=5.3 Hz), 130.8, 127.7, 122.5 (q, J=271 Hz), 121.6, 117.4, 109.0, 106.6, 104.5, 101.6, 55.4.

The following compounds were made according to the procedure of Example 3—Step 1:

3-(2-Benzyloxy-5-methoxy-phenyl)-1H-pyrazole (P52): [M+H]⁺ 281.2; ¹H (400 MHz, DMSO-d6) δ 7.64 (bs, 1H), 7.50-7.43 (m, 2H), 7.36 (dd, J=7.6, 7.4 Hz, 1H), 7.32-7.28 (m, 2H), 7.08 (d, J=8.8 Hz, 1H), 6.84-6.80 (m, 2H), 6.70 (bs, 1H), 5.41 (s, 2H), 3.73 (s, 3H):

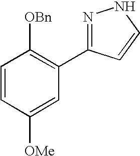

The following compounds were made according to Example 3—Step 2:

2-[5-(2-Benzyloxy-5-methoxy-phenyl)-pyrazol-1-yl]-3-trifluoromethyl-pyridine (81): [M+H]⁺ 426.2; ¹H (400 MHz, CDCl₃) δ 8.54 (dd, J=4.4, 1.6 Hz, 1H), 8.07 (dd, J=8.0, 2.0 Hz, 1H), 7.83 (d, J=1.6 Hz, 1H), 7.44 (dd, J=8.4, 7.6 Hz, 1H), 7.30-7.24 (m, 2H), 7.10-7.06 (m, 2H), 6.79 (d, J=6.8 Hz, 1H), 6.76-6.69 (m, 4H), 6.63 (d, J=2.0 Hz, 1H), 4.81 (s, 2H), 3.66 (s, 3H):

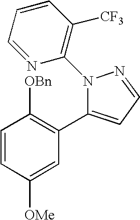

2-[5-(2-Benzyloxy-5-bromo-phenyl)-pyrazol-1-yl]-3-trifluoromethyl-pyridine (107): ¹H (400 MHz, CDCl₃) δ 8.47 (dd, J=4.8, 1.6 Hz, 1H), 8.06 (dd, J=8.0, 1.6 Hz, 1H), 7.79. (d, J=2.0 Hz, 1H), 7.44 (d, J=2.4 Hz, 1H), 7.39 (dd, J=7.6, 4.8 Hz, 1H), 7.30-7.24 (m, 4H), 7.00-6.97 (m, 2H), 6.62 (d, J=8.8 Hz, 1H), 6.57 (d, J=2.0 Hz, 1H), 4.79 (s, 2H):

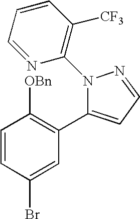

2-[3-(2-Benzyloxy-5-methoxy-phenyl)-pyrazol-1-ylmethyl]-pyridine (114): ¹H (400 MHz, CDCl₃) δ 8.63 (d, J=5.2 Hz, 1H), 7.72 (dd, J=7.6, 7.4 Hz, 1H), 7.60 (d, J=3.2 Hz, 1H), 7.51 (d, J=2.4 Hz, 1H), 7.45-7.29 (m, 6H), 7.13 (d, J=8.0 Hz, 1H), 6.96 (d, J=8.8 Hz, 1H), 6.89 (d, J=2.0 Hz, 1H), 6.82 (dd, J=8.8, 3.2 Hz, 1H), 5.89 (s, 2H), 5.10 (s, 2H), 3.83 (s, 3H):

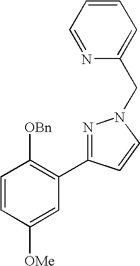

2-[5-(2-Benzyloxy-5-methoxy-phenyl)-pyrazol-1-ylmethyl]-pyridine (116): [M+H]⁺ 372.2:

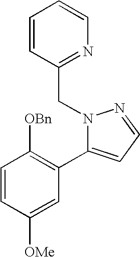

2-[5-(2-Benzyloxy-5-methoxy-phenyl)-pyrazol-1-ylmethyl]-quinoline 110: [M+H]⁺ 422.3:

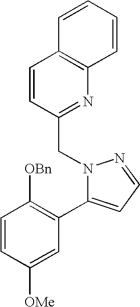

2-[3-(2-Benzyloxy-5-methoxy-phenyl)-pyrazol-1-ylmethyl]-3,4-dimethoxy-pyridine (103):
[M+H]⁺ 432.3:

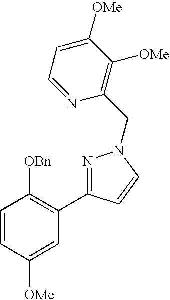

2-[3-(2-Benzyloxy-5-methoxy-phenyl)-pyrazol-1-ylmethyl]-quinoline (112): [M+H]⁺ 422.3:

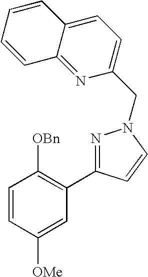

The following compounds were made according to the procedure of Example 3—Step 3:

4-Bromo-2-[1-(3-trifluoromethyl-pyridin-2-yl)-1H-pyrazol-3-yl]-phenol (100): [M+H]+384.2; ¹H (400 MHz, CDCl₃) δ 10.34 (bs, 1H), 8.68 (d, J=4.4 Hz, 1H), 8.45 (d, J=2.4 Hz, 1H), 8.24 (d, J=8.0 Hz, 1H), 7.73 (d, J=2.0 Hz, 1H), 7.46 (dd, J=7.6, 4.8 Hz, 1H), 7.34 (dd, J=8.8, 2.0 Hz, 1H), 6.95 (d, J=8.4 Hz, 1H), 6.88 (d, J=2.8 Hz, 1H):

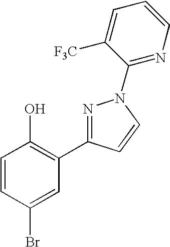

4-Bromo-2-[2-(3-trifluoromethyl-pyridin-2-yl)-2H-pyrazol-3-yl]-phenol (106): [M+H]+384.2; 1H (400 MHz, CDCl₃) δ 8.60 (dd, J=5.2, 1.2 Hz, 1H), 8.23 (dd, J=8.0, 2.0 Hz, 1H), 7.84 (d, J=1.6 Hz, 1H), 7.58 (dd, J=8.0, 5.2 Hz, 1H), 7.37 d(d, J=8.8, 2.8 Hz, 1H), 7.24 (d, J=2.4 Hz, 1H), 6.87 (d, J=8.8 Hz, 1H), 6.48 (d, J=1.2 Hz, 1H):

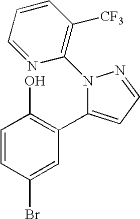

4-Methoxy-2-[1-(3-trifluoromethyl-pyridin-2-yl)-1H-pyrazol-3-yl]-phenol (86): ¹H (400 MHz, CDCl₃) δ 9.93 (bs, 1H), 8.65 (d, J=4.8 Hz, 1H), 8.43 (d, J=2.8 Hz, 1H), 8.22 (d, J=8.0 Hz, 1H), 7.43 (dd, J=8.0, 4.8 Hz, 1H), 7.15 (d, J=3.2 Hz, 1H), 6.99 (d, J=8.8 Hz, 1H), 6.89-6.86 (m, 2H), 3.84 (s, 3H):

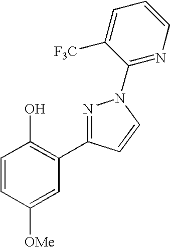

4-Methoxy-2-[1-(3-nitro-pyridin-2-yl)-1H-pyrazol-3-yl]-phenol (117): [M+H]⁺ 313.3; ¹H (400 MHz, CDCl₃) δ 9.08 (bs, 1H), 8.60 (dd, J=4.8, 1.6 Hz, 1H), 8.53 (d, J=3.2 Hz, 1H), 8.04 (dd, J=8.4, 1.6 Hz, 1H), 7.39 (dd, J=8.0, 4.8 Hz, 1H), 7.10 (d, J=3.2 Hz, 1H), 6.99 (d, J=4.8 Hz, 1H), 6.89-6.86 (m, 2H), 3.83 (s, 3H); 1³C (100 MHz, CDCl₃) 5154.8, 152.4, 150.3, 149.9, 133.4, 129.8, 128.7, 127.6, 121.7, 118.1, 116.6, 115.3, 112.0, 106.0, 56.0:

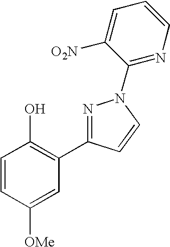

2-[1-(3,4-Dimethoxy-pyridin-2-ylmethyl)-1H-pyrazol-3-yl]-4-methoxy-phenol (105): [M+H]+342.3; ¹H (400 MHz, CDCl₃) δ 8.46 (d, J=6.0 Hz, 1H), 7.88 (d, J=2.0 Hz, 1H), 7.21 (d, J=6.4 Hz, 1H), 7.02 (d, J=3.5 Hz, 1H), 6.85 (d, J=8.8 Hz, 1H), 6.76 (d, J=8.8 Hz, 1H), 6.59 (dd, J=2.8 Hz, 1H), 5.69 (s, 2H), 4.10 (s, 3H), 4.02 (s, 3H), 3.78 (s, 3H):

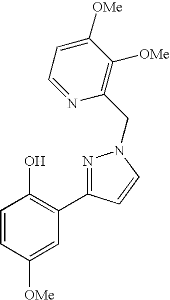

4-Methoxy-2-(1-pyridin-2-ylmethyl-1H-pyrazol-3-yl)-phenol (113): [M+H]⁺ 282.3; ¹H (400 MHz, CDCl₃) δ 9.01 (bs, 1H), 8.63 (d, J=4.8 Hz, 1H), 8.15 (dd, J=8.0, 7.6 Hz, 1H), 7.76 (d, J=8.0 Hz, 1H), 7.71 (d, J=1.6 Hz, 1H), 7.63 (dd, J=7.2, 6.0 Hz, 1H), 6.95 (d, J=8.8 Hz, 1H), 6.88 (dd, J=9.2, 3.2 Hz, 1H), 6.69 (d, J=3.2 Hz, 1H), 6.40 (d, J=2.0 Hz, 1H), 5.78 (s, 2H), 3.76 (s, 3H):

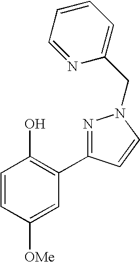

4-Methoxy-2-(2-pyridin-2-ylmethyl-2H-pyrazol-3-yl)-phenol (115): [M+H]⁺ 282.3:

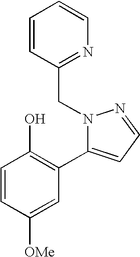

4-Methoxy-2-(1-quinolin-2-ylmethyl-1H-pyrazol-3-yl)-phenol (111): [M+H]⁺ 332.3;. ¹H (400 MHz, CDCl₃) δ 8.26 (d, J=8.8 Hz, 1H), 8.22 (d, J=8.4 Hz, 1H), 7.86 (d, J=8.0 Hz, 1H), 7.83 (dd, J=7.2, 6.8 Hz, 1H), 7.70 (d, J=2.4 Hz, 1H), 7.63 (dd, J=7.6, 7.2 Hz, 1H), 7.30 (d, J=8.8 Hz, 1H), 7.10 (d, J=2.8 Hz, 1H), 6.94 (d, J=8.8 Hz, 1H), 6.82 (dd, J=8.8, 3.2 Hz, 1H), 6.69 (d, J=2.0 Hz, 1H), 5.80 (s, 2H), 3.82 (s,3H):

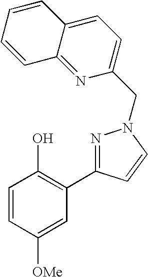

4-Methoxy-2-(2-quinolin-2-ylmethyl-2H-pyrazol-3-yl)-phenol (109): [M+H]⁺ 332.3; ¹H (400 MHz, CDCl₃) δ 8.52 (d, J=8.4 Hz, 1H), 8.22 (d, J=8.0 Hz, 1H), 7.97 (d, J=8.4 Hz, 1H), 7.92 (dd, J=7.2,1.6 Hz, 1H), 7.76 (dd, J=8.0,7.6 Hz, 1H), 7.72 (d, J=2.0 Hz, 1H), 7.67 (d, J=8.4 Hz, 1H), 6.91 (d, J=8.8 Hz, 1H), 6.77 (dd, J=8.8, 3.2 Hz, 1H), 6.62 (d, J=3.2 Hz, 1H), 6.42 (d, J=2.0 Hz, 1H), 5.88 (s, 2H), 3.67 (s, 3H):

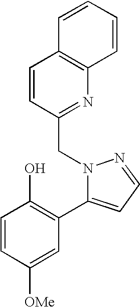

4-Methoxy-2-[1-(4-methoxy-3,5-dimethyl-pyridin-2-ylmethyl)-1H-pyrazol-3-yl]-phenol (101): [M+H]⁺ 340.3; ¹H (400 MHz, CDCl₃) δ 8.49 (s, 1H), 7.95 (d, J=2.4 Hz, 1H), 7.04 (d, J=3.2 Hz, 1H), 6.89 (d, J=8.8 Hz, 1H), 6.80 (dd, J=8.8,2.8 Hz, 1H), 6.62 (d, J=2.8 Hz, 1H), 5.77 (s, 2H), 4.03 (s, 3H), 3.80 (s, 3H), 2.61 (s, 3H), 2.43 (s, 3H):

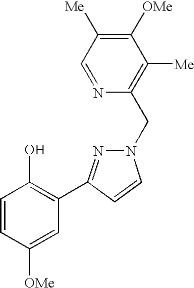

{6-[3-(2-Hydroxy-5-methoxy-phenyl)-pyrazol-1-yl]-pyridin-3-yl)-piperidin-1-yl-methanone (102): [M+H]⁺ 379.4; ¹H (400 MHz, CDCl₃) δ 10.10 (bs, 1H), 8.62 (d, J=3.5 Hz, 1H), 8.48 (d, J=1.2 Hz, 1H), 7.9007.87 m, 2H), 7.13 (d, J=2.4 Hz, 1H), 6.98 (d, J=8.0 Hz, 1H), 6.89-6.84 (m, 2H), 3.83 (s, 3H), 3.77 (bs, 2H), 3.43 (bs, 2H), 1.71-1.60 (m, 6H); ¹³C (100 MHz, CDCl₃) δ 166.8, 153.9, 152.5, 150.5, 150.0, 146.8, 137.9, 129.8, 128.2, 117.6, 116.1, 115.8, 111.7, 111.3, 105.4, 55.9, 49.0, 43.5, 26.6, 25.6, 24.5:

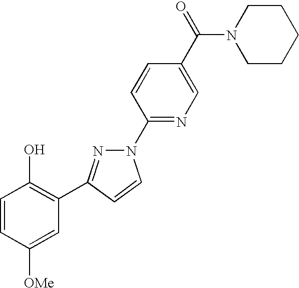

{2-[3-(2-Hydroxy-5-methoxy-phenyl)-pyrazol-1-yl]-pyridin-3-yl}-piperidin-1-yl-methanone (104): [M+H]⁺ 379.4; ¹H (400 MHz, CDCl₃) δ 8.59 (d, J=3.6 Hz, 1H), 8.46 (d, J=5.6 Hz, 1H), 7.75 (d, J=8.0 Hz, 1H), 7.30-7.26 (m, 1H), 7.11 (d, J=2.8 Hz, 1H), 6.98 (d, J=8.8 Hz, 1H), 6.87-6.22 (m, 2H), 4.11-4.05 (m, 1H), 3.82 (s, 3H), 3.59-3.52 (m, 1H), 3.25-3.18 (m, 1H), 2.99-2.92 (m, 1H), 1.75-1.61 (m, 2H), 1.61-1.45 (m, 2H), 1.41-1.30 (m, 1H), 1.30-1.20 (m, 1H); ¹³C (100 MHz, CDCl₃) δ 166.4, 153.5, 152.3, 149.9, 148.4, 145.7, 137.8, 129.2, 122.5, 121.6, 117.8, 115.9, 115.8, 111.7, 105.0, 55.9, 47.8, 42.7, 25.8, 25.1, 24.3:

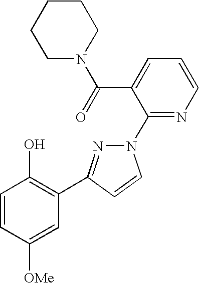

### EXAMPLE 4

Synthesis of 8-Methoxy-3-methyl-2-pyridin-2-yl-2H-chromeno[4,3-clpyrazol-4-one (94) and 3-Methyl-2-pyridin-2-yl-2H-chromeno[4,3-clpyrazol-4-one (95).

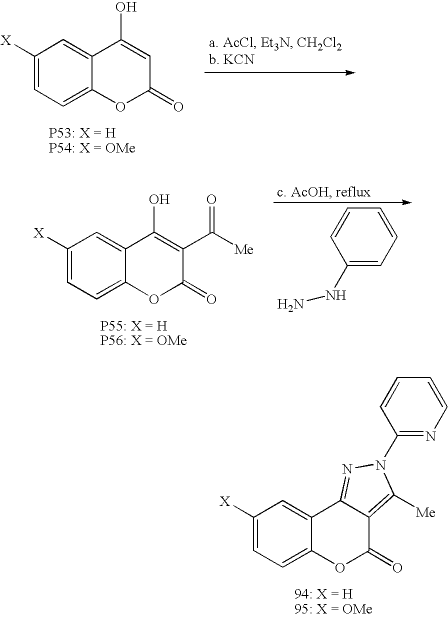

Step 1: Preparation of 3-Acetyl-4-hvdroxy-6-methoxy-chromen-2-one (P56): To a suspension of 4-hydroxy-6-methoxycoumarin P54 (0.0156 moles, 3 g) in CH₂Cl₂ (0.25M, 60 mL) was added triethylamine (5.0 equiv, 0.078 moles, 11 mL) and the reaction mixture was stirred for 15 min at ambient temperature, leading to a homogeneous solution. To that was added acetylchloride (1.2 equiv, 0.0187 moles, 1.33 nmL) dropwise and the whole was stirred for 3 h at ambient temperature. Solid potassium cyanide (3.0 equiv, 0.0468 moles, 3.05 g) was added and stirring was continued for 2 days. The reaction mixture was quenched with HCl 6M to pH 2 and extracted with CH₂Cl₂ (x3). The combined organic extracts were dried with MgSO₄ and concentrated. Recrystallization from EtOH produced P56 in 56% yield. [M+H]⁺ 235.1; ¹H (400 MHz, DMSO-d6) δ 7.39 (dd, J=9.2, 2.8 Hz, 1H), 7.37 (s, 1H), 7.34 (dd, J=3.6, 3.2 Hz, 1H), 3.83 (s, 3H), 2.66 (s, 3H); ¹³C (100 MHz, DMSO) δ 206.0, 178.1, 156.3, 149.3, 147.3, 125.6, 119.0, 115.7, 106.5, 102.0, 56.6, 30.4.

Step 2: Preparation of 8-Methoxy-3-methyl-2-pyridin-2-yl-2H-chromeno[4,3-clpyrazol-4-one (95): The procedure described in example 1—step 3 was followed. [M+H]⁺ 308.3; 1H (400 MHz, CDCl₃) δ 8.50 (d, J=4.0 Hz, 1H), 8.05 (d, J=8.4 Hz, 1H), 7.91 (dd, J=8.0, 7.6 Hz, 1H), 7.63 (d, J=9.2 Hz, 1H), 7.43 (d, J=2.8 Hz, 1H), 7.30-7.23 (m, 2H), 3.89 (s, 3H), 2.77 (s, 3H).

The following compounds were prepared according to the procedure of Example 4—Step 1:

3-Acetyl-4-hydroxy-chromen-2-one (P55): ¹H (400 MHz, DMSO-d6) δ 8.00 (dd, J=8.0, 1.2 Hz, 1H), 7.82 (ddd, J=8.0, 7.2, 1.6 Hz, 1H), 7.45-7.40 )m, 2H), 2.67 (s, 3H):

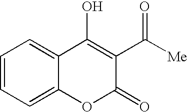

The following compounds were prepared according to the procedure of Example 4—Step 2:

3-Methyl-2-pyridin-2-yl-2H-chromeno[4,3-clpyrazol-4-one (94): [M+H]⁺ 278.2; ¹H (400 MHz, CDCl₃) δ 8.70 (d, J=5.6 Hz, 1H), 8.57 (d, J=8.4 Hz, 1H), 8.30 (d, J=7.6 Hz, 1H), 8.06 (dd, J=7.6, 7.2 Hz, 1H), 7.65-7.61 (m, 1H), 7.54-7.49 (m, 2H), 7.34 (ddd, J=5.6, 5.4, 0.8 Hz, 1H), 2.88 (s, 3H):

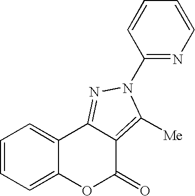

### EXAMPLE 5

2-(5-Methyl-1-pyridin-2-yl-1H-pyrazol-3-yl)-phenol (P58) and 2-(5-Methyl-2-pyridin-2-yl-2H-pyrazol-3-yl)-phenol (21).

A mixture of diketone P57 (50 mg, 0.28 mmoles) and 3-nitro-2-pyridyl hydrazine (2.2 equiv, 0.62 mmoles, 115 mg) was dissolved in ethanol and the whole was refluxed for 16 h. Evaporation of the solvent under reduced pressure, followed by chromatographic purification, fuinished 1,3-pyrazole P58 (37%) and 1,2-pyrazole 21 (29%).For P58: Rf 0.83 (100% CH₂Cl₂); [M+H]⁺ 296.2; ¹H (400 MHz, CDCl₃) δ 11.02 (s, 1H), 8.23 (d, J=7.6 Hz, 1H), 8.16 (d, J=8.8 Hz, 1H), 7.51 (dd, J=7.2, 6.4 Hz, 1H), 7.41 (dd, J=8.0, 6.8 Hz, 1H), 7.29-7.17 (m, 2H), 6.76 (dd, J=6.4, 6.4 Hz, 1H), 6.11 (s, 1H), 2.32 (s, 3H). For 21: Rf 0.71 (100% CH₂Cl₂); ); [M+H]+296.1:

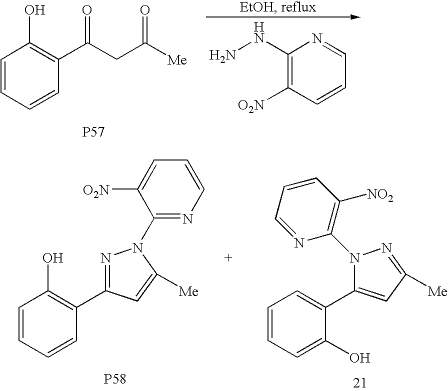

The following compounds were prepared according to the above procedure:

1-(5-Methyl-1-phenyl-1H-pyrazol-3-yl)-naphthalen-2-ol (4): [M+H]⁺ 301.2:

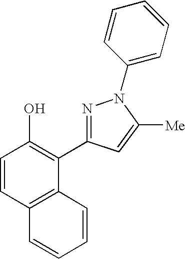

1-[5-Methyl-1-(2-trifluoromethyl-phenyl)-1H-pyrazol-3-yl]-naphthalen-2-ol (5): [M+H]+369.3; ¹H (400 MHz, CDCl₃) δ 7.72 (dd, J=9.2, 8.0 Hz, 2H), 7.63 (d, J=7.6 Hz, 1H), 7.53 (d, J=8.8 Hz, 1H), 7.39 (dd, J=7.6, 7.2 Hz, 1H), 7.32-7.21 (m, 2H), 7.17 (dd, J=8.0, 6.8 Hz, 1H), 7.10-7.02 (m, 2H), 6.44 (s, 1H), 2.49 (s, 3H):

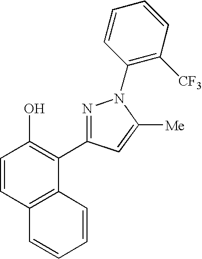

1-[5-Methyl-2-(2-trifluoromethyl-phenyl)-2H-pyrazol-3-yl]-naphthalen-2-ol (6): [M+H]+369.3.

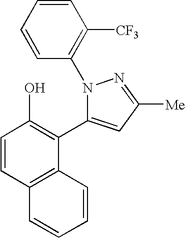

1-[5-Methyl-1-(2-nitro-phenyl)-1 H-pyrazol-3-yl]-naphthalen-2-ol (1): [M+H]⁺ 346.2:

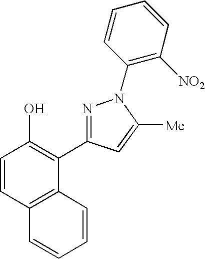

2-[5-Methyl-1-(2-nitro-phenyl)-1H-pyrazol-3-yl]-phenol (2): ¹H (400 MHz, CDCl₃) δ 11.04 (s, 1H), 8.22 (dd, J=8.0, 1.6 Hz, 1H), 8.16 (dd, J=8.8, 1.6 Hz, 1H), 8.01 (dd, J=8.4, 0.8 Hz, 1H), 7.55-7.50 (m, 1H), 7.41 (ddd, J=7.2, 6.8, 1.2 Hz, 1H), 7.27 (ddd, J=7.6, 7.2, 6.8, 0.8 Hz, 1H), 7.18 (dd, J=8.8, 1.2 Hz, 1H), 6.76 (ddd, J=8.0, 6.8, 0.8 Hz, 1H), 6.19 (s, 1H), 2.31 (s, 3H):

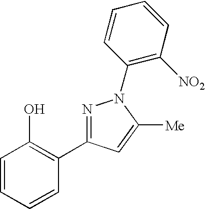

2-[5-Methyl-2-(2-nitro-phenyl)-2H-pyrazol-3-yl]-phenol (3): [M+H]⁺ 296.2:

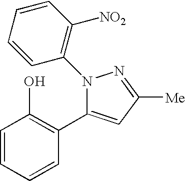

2-(5-Methyl-1-phenyl-1H-pyrazol-3-yl)-phenol (11): [M+H]⁺ 251.1; ¹H (400 MHz, CDCl₃) δ 10.89 (s, 1H), 7.58 (dd, J=8.0, 2.0 Hz, 1H), 7.52-7.48 (m, 4H), 7.44-7.41 (m, 2H), 7.22 (ddd, J=8.4, 8.4, 1.6Hz, 1H), 7.03 (dd, J=8.4, 1.2Hz, 1H), 6.92 (ddd, J=7.6, 7.2, 1.2 Hz, 1H), 2.44 (s, 3H):

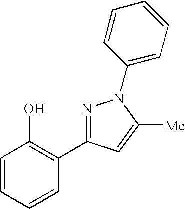

2-[1-(4-Methoxy-phenyl)-5-methyl-1H-pyrazol-3-yl]-phenol (14): [M+H]⁺ 281.3; H (400 MHz, CDCl₃) δ 7.20-7.13 (m, 1H), 7.14 (d, J=8.8 Hz, 2H), 6.94 (d, J=8.0, 2.0 Hz, 1H), 6.85 (d, J=8.0 Hz, 1H), 6.81-6.74 (m, 1H), 6.75 (d, J=8.8 Hz, 2H), 6.31 (s, 1H), 6.22 (bs, 1H), 3.75 (s, 3H), 2.37 (s, 3H):

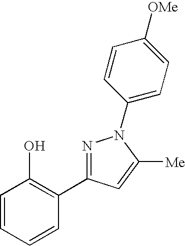

2-[5-Methyl-i-(2-trifluoromethyl-phenyl)-1H-pyrazol-3-yl]-phenol (7): [M+H]⁺ 319.2; ¹H (400 MHz, CDCl₃) δ 510.62 (s, 1H), 7.85 (dd, J=8.0,1.6 Hz, 1H), 7.71-7.65 (m, 2H), 7.57 (dd, J=7.2,1.2 Hz, 1H), 7.40 (d, J=7.2 Hz, 1H), 7.19 (dd, J=8.8,2.0 Hz, 1H), 6.98 (d, J=8.0 Hz, 1H), 6.91 (ddd, J=8.0, 8.0, 0.8 Hz, 1H), 6.60 (s, 1H), 2.19 (s, 3H):

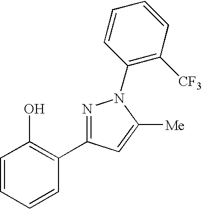

2-[5-Methyl-1-(2-nitro-phenyl)-1H-pyrazol-3-yl]-naphthalen-1-ol 12: [M+H]⁺ 346.2:

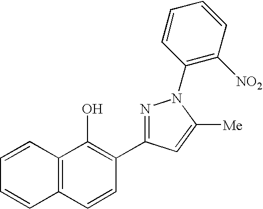

1-[1-(4-Chloro-phenyl)-5-methyl-1H-pyrazol-3-yl]-naphthalen-2-ol (18): [M+H]⁺ 337.2:

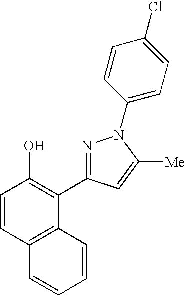

1-[1-(4-Methoxy-phenyl)-5-methyl-1H-pyrazol-3-ylj-naphthalen-2-ol (8): [M+H]⁺ 331.2:

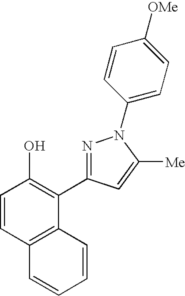

1-[1-(4-Isopropyl-phenyl)-5-methyl-1H-pyrazol-3-yl]-naphthalen-2-ol (13): [M+H]⁺ 343.3:

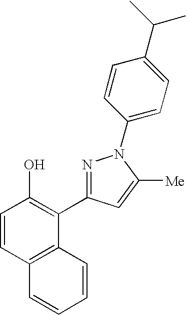

2-[2-(4-Chloro-phenyl)-5-methyl-2H-pyrazol-3-yl]-naphthalen-1-ol (19): [M+H]⁺ 335.2; ¹H (400 MHz, CDCl₃) δ 8.30-8.23 (m, 1H), 7.78-7.75 (m, 1H), 7.55-7.52 (m, 2H), 7.29 (d, J=8.8 Hz, 1H), 7.20 (s, 4H), 6.90 (d, J=8.8 Hz, 1H), 6.45 (s, 1H), 6.13 (s, 1H), 2.45 (s, 3H); ¹³C (100 MHz, CDCl₃) δ 150.5, 149.7, 138.3, 138.2, 134.8, 132.7, 129.1, 127.7, 127.5, 126.9, 126.0, 125.1, 124.4, 122.8, 120.4, 110.2, 109.0, 14.1:

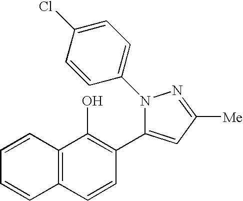

2-(5-Methyl-2-pyridin-2-yl-2H-pyrazol-3-yl)-naphthalen-1-ol (9): [M+H]⁺ 302.2; ¹H (400 MHz, CDCl₃) δ 11.02 (s, 1H), 8.56-8.50 (m, 1H), 8.30-8.25 (m, 1H), 7.90-7.75 (m, 2H), 7.60-7.48 (m, 3H), 7.37 (d, J=8.4 Hz, 1H), 7.25-7.20 (m, 1H), 7.19 (d, J=8.4 Hz, 1H), 6.26 (s, 1H), 2.43 (s, 3H):

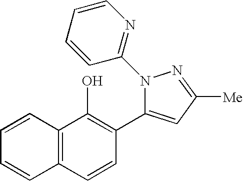

2-[5-Methyl-1-(2-trifluoromethyl-phenyl)-1H-pyrazol-3-yl]-naphthalen-1-ol (16): [M+H]+369.2:

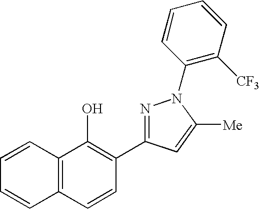

2-[1-(4-Isopropyl-phenyl)-5-methyl-1H-pyrazol-3-yl]-naphthalen-1-ol (29): [M+H]⁺ 343.3:

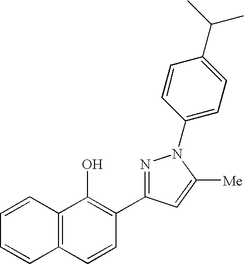

4,5-Dimethyl-2-(5-methyl-1-phenyl-1H-pyrazol-3-yl)-phenol (17): [M+H]⁺ 279.3:

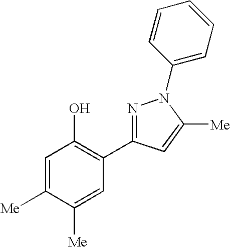

2-[1-(4-Chloro-phenyl)-5-methyl-1H-pyrazol-3-yl]-4,5-dimethyl-phenol (22): [M+H]⁺ 313.3; ¹H (400 MHz, CDCl₃) δ 10.43 (s, 1H), 7.48-7.41 (m, 3H), 7.29 (s, 1H), 7.25 (s, 1H), 6.81 (s, 1H), 6.56 (s, 1H), 2.41 (s, 3H), 2.25 (s, 3H), 2.24 (s, 3H):

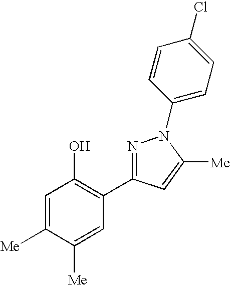

4,5-Dimethyl-2-[5-methyl-i-(2-trifluoromethyl-phenyl)-1H-pyrazol-3-yl]-phenol (24): [M+H]⁺ 347.3; ¹H (400 MHz, CDCl₃) δ 7.84 (d, J=7.6 Hz, 1H), 7.70-7.62 (m, 3H), 7.40 (d, J=8.0 Hz, 1H), .7.30 (s, 1H), 6.79 (s, 1H), 6.53 (s, 1H), 2.25 (s, 3H), 2.24 (s, 3H), 2.18 (s, 3H):

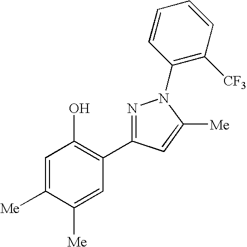

4,5-Dimethyl-2-(5-methyl-2-o-tolyl-2H-pyrazol-3-yl)-phenol (10): [M+H]⁺ 293.3:

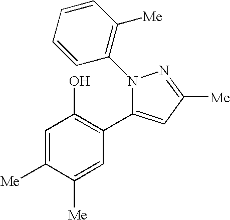

2-[1-(2-Fluoro-phenyl)-5-methyl-1H-pyrazol-3-yl]-4,5-dimethyl-phenol (15): [M+H]⁺ 297.3:

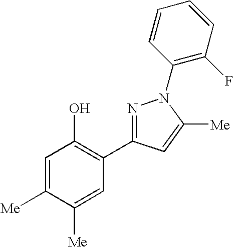

2-[1-(4-Isopropyl-phenyl)-5-methyl-1H-pyrazol-3-yl]-4,5-dimethyl-phenol (30): [M+H]+321.3; ¹H (400 MHz, CDCl₃) δ 7.25 (s, 1H), 7.17 (d, J=8.0 Hz, 2H), 7.12 (d, J=8.0 Hz, 2H), 6.71 (d, J=2.8 Hz, 1H), 6.27 (s, 1H), 2.92-2.82 (m, 1H), 2.39 (s, 3H), 2.22 (s, 3H), 2.06 (s, 3H), 1.21 (d, J=7.2 Hz, 6H):

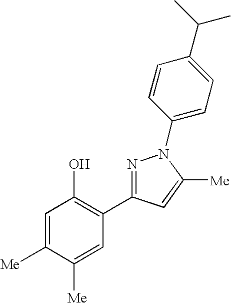

2-(l-Benzothiazol-2-yl-5-methyl-1H-pyrazol-3-yl)-4,5-dimethyl-phenol (23): [M+H]⁺ 336.3; ¹H (400 MHz, CDCl₃) δ 8.71 (bs 1H), 7.80-7.77 (m, 2H), 7.41 (ddd, J=8.8, 8.0, 1.2 Hz, 1H), 7.33 (ddd, J=8.8, 8.4, 1.2 Hz, 1H), 7.01 (d, J=6.4 Hz, 2H), 6.28 (s, 1H), 2.41 (s, 3H), 2.30 (s, 3H), 2.21 (s, 3H):

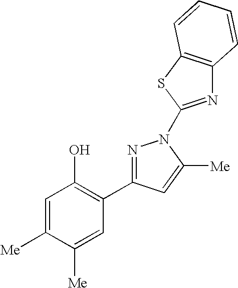

1-[5-Methyl-1-(3-trifluoromethyl-pyridin-2-yl)-1H-pyrazol-3-yl]-naphthalen-2-ol (26): [M+H]⁺ 370.2:

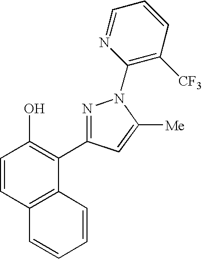

1-[5-Methyl-i -(6-methyl-4-trifluoromethyl-pyridin-2-yl)-1 H-pyrazol-3-yl]-naphthalen-2-ol (27): [M+H]⁺ 384.1:

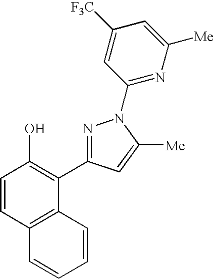

1-(5-Methyl-1-pyridin-2-yl-1H-pyrazol-3-yl)-naphthalen-2-ol (28): [M+H]⁺ 302.2:

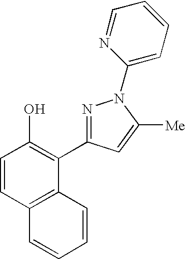

1-[1-(3-Chloro-5-trifluoromethyl-pyridin-2-yl)-5-methyl-1H-pyrazol-3-yl]-naphthalen-2-ol (20): [M+H]⁺ 404.1; ¹H (400 MHz, CDCl₃) δ 10.40 (bs, 1H), 8.77 (s, 1H), 8.30 (d, J=8.4 Hz, 1H), 8.23 (s, 1H), 7.82-7.75 (m, 2H), 7.50 (ddd, J=8.0,7.6,1.2 Hz, 1H), 7.36 (ddd, J=8.0, 7.4, 1.2 Hz, 1H), 7.27 (d, J=8.8 Hz, 1H), 6.82 (s, 1H), 2.55 (s, 3H):

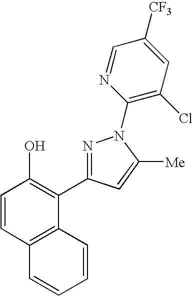

1-[5-Methyl-1-(3-nitro-pyridin-2-yl)-1H-pyrazol-3-yl]-naphthalen-2-ol (25): [M+H]⁺ 347.2:

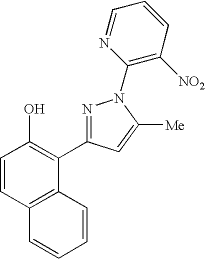

### EXAMPLE 6

Synthesis of 5-(4-Methoxy-phenyl)-2-pyridin-2-yl-2H-pyrazole-3-carboxylic acid ethyl ester (P59), 5-(4-Methoxy-phenyl)-1-pyridin-2-vI-1H-pyrazole-3-carboxylic acid ethyl ester (P60), 5-(4-Methoxy-phenyl)-2-pyridin-2-yl-2H-pyrazole-3-carboxylic acid (63) and 5-(4-Methoxy-phenyl)-2-pyridin-2-yl-2H-pyrazole-3-carboxylic acid propylamide (62).

Step 1: Preparation of 5-(4-Methoxy-phenyl)-2-pyridin-2-yl-2H-pyrazole-3-carboxylic acid ethyl ester (P59) and 5-(4-Methoxy-phenyl)-1-pyridin-2-yl-1H-pyrazole-3-carboxylic acid ethyl ester (P60).

To a stirred solution of 4-(4-Methoxy-phenyl)-2,4-dioxo-butyric acid ethyl ester (P58) (0.150 g, 0.6 mmol) in ethanol/toluene/acetic acid (6:6:1, 14 mL) was added pyridine-2-yl hydrazine hydrochloride (0.131 g, 1.2 mmol) and trifluoroacetic acid (0.1 mL). After stirring in a sealed vial at 80° C. for 16 h the solution was allowed to cool to room temperature, transferred to a pear shaped flask, concentrated and chromatographed to give the title compounds as a mixture that was separated by HPLC. [M+H]⁺ 324.2; for P59: ¹H NMR (CDCl₃) δ 8.40-8.42 (m, 1 H), 7.79 (td, J=2.0, 8.4 Hz, 1 H), 7.53 (dd, J=0.8, 8.0 Hz, 1 H), 7.29 (ddd, J=0.8, 4.8, 8.4 Hz, 1 H), 7.14-7.17 (m, 2 H), 6.98 (s, 1 H), 6.80-6.85 (m, 2 H), 4.45 (q,J=7.6Hz, 2 H), 3.81 (s, 3 H), 1.43 (t, J=7.6 Hz, 3 H); for P60: ¹H NMR (CDCl₃) δ 8.48 (dd, J=1.2, 4.8 Hz, 1 H), 7.87 (td, J=2.0, 8.0 Hz, 1 H), 7.79-7.82 (m, 2 H), 7.74 (d, J=8.0 Hz, 1 H), 7.31 (ddd, J=0.8, 4.8, 8.0 Hz, 1 H), 7.13 (s, 1 H), 6.94-6.97 (m, 2 H), 4.32 (q, J=7.2 Hz, 2 H), 3.85 (s, 3 H), 1.28 (t, J=7.2 Hz,3H).

Step 2: Preparation of 5-(4-Methoxy-phenyl)-2-pyridin-2-yl-2H-p3razole-3-carboxylic acid (63).

To 5-(4-Methoxy-phenyl)-2-pyridin-2-yl-2H-pyrazole-3-carboxylic acid ethyl ester (0.028 g), dissolved in wet methanol (5% H₂0) in an 8 mL vial was added K₂CO₃ (0.050 g) and the suspension was shaken at 60° C. overnight. The resulting solution was acidified with 6M HCl (aq) and diluted with ethyl acetate (10 mL). The organic phase was washed with brine, dried (MgSO₄) and concentrated to give the title compound (0.021 g) as a colorless solid. [M+H]⁺ 296.0

Step 3: Preparation of 5-(4-Methoxy-phenyl)-2-pyridin-2-yl-2H-pyrazole-3-carboxylic acid propylamide (62).

5-(4-Methoxy-phenyl)-2-pyridin-2-yl-2H-pyrazole-3-carboxylic acid ethyl ester (0.025 g) was placed in an 8 mL vial, dissolved in propylamine (2 mL), capped and heated to 85° C. with stirring for 16 h. The solution was concentrated to dryness to give an oil that was co-evaporated with ethanol (3×5 mL). The remaining solid was shown to be pure by ¹H NMR. ¹H NMR (CDCl₃) δ 8.49-8.51 (m, 1 H), 7.73 (td, J=2.0, 8.0 Hz, 1 H), 7.25-7.31 (m, 2 H), 7.13-7.16 (m, 2 H), 7.04-7.07 (m, 1 H), 6.99 (s, 1 H), 6.81-6.85 (m, 2 H), 3.80 (s, 3 H), 3.42 (q, J=6.0 Hz, 2 H), 1.61-1.67 (m, 2 H), 0.99 (t, J=7.2 Hz, 3 H):

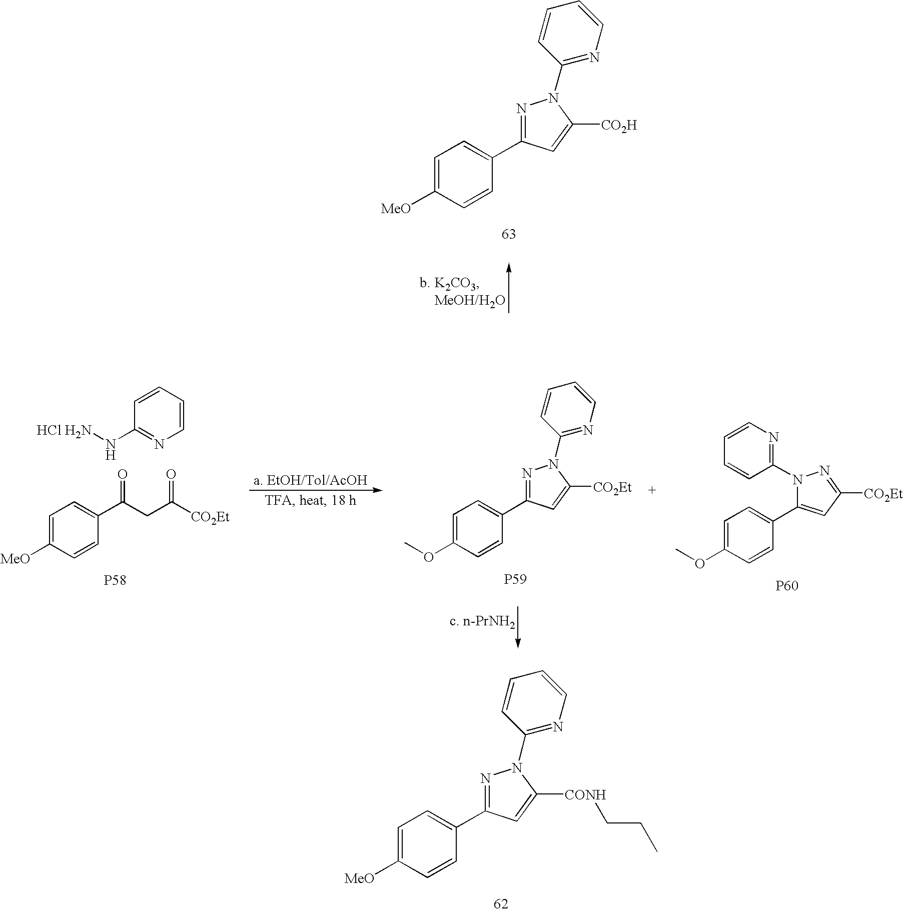

The following compounds were made by the procedure of Example 6—Step 1:

2-(4-Chloro-phenyl)-5-(4-methoxy-phenyl)-2H-pyrazole-3-carboxylic acid ethyl ester (P61): Prepared according to the same procedure as 5-(4-Methoxy-phenyl)-2-pyridin-2-yl-2H-pyrazole-3-carboxylic acid ethyl ester. ¹H NMR (CDCl₃) δ 7.25-7.34 (m, 4 H), 7.10-7.14 (m, 2 H), 6.96 10 (s, 1 H), 6.82-6.86 (m, 2 H), 4.45 (q, J=7.6 Hz, 2 H), 3.81 (s, 3 H), 1.43 (t, J=7.6 Hz, 3 H):

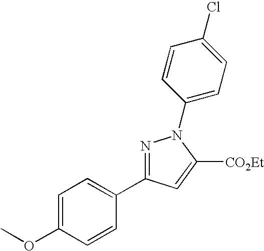

Compound 56: 5-(4-Methoxy-phenyl)-2-(3-trifluoromethyl-pyridin-2-yl)-2H-pyrazole-3-carboxylic acid ethyl ester; and

Compound 57: 5-(4-Methoxy-phenyl)-1-(3-trifluoromethyl-pyridin-2-yl)-1H-pyrazole-3-carboxylic acid ethyl ester: Prepared according to the same procedure as 5-(4-Methoxy-phenyl)-2-pyridin-2-yl-2H-pyrazole-3-carboxylic acid ethyl ester. For 56: [M+H]⁺ 392.2 ¹H NMR (CDCl₃) δ 8.72 (dd, J=2.0, 4.8 Hz, 1 H), 8.12 (dd, J=2.0, 8.0 Hz, 1 H), 7.56 (dd, J=4.8, 8.0 Hz, 1 H), 7.11-7.14 (m, 2 H), 7.01 (s, 1 H), 6.75-6.78 (m, 2 H), 4.45 (q, J=7.6 Hz, 2 H), 3.52 (s, 3 H), 1.40 (t, J=7.6 Hz, 3 H); for 57: ¹H NMR (CDCl₃) δ 8.79 (dd, J=2.0, 4.8 Hz, 1 H), 8.20 (dd, J=2.0, 8.0 Hz, 1 H), 7.76-7.81 (m, 2 H), 7.61 (dd, J=4.8, 8.0 Hz, 1 H), 7.25 (s, 1 H), 6.92-6.96 (m, 2 H), 4.21 (q, J=7.6Hz, 2 H), 3.85 (s, 3 H), 1.22 (t, J=7.6Hz, 3 H):

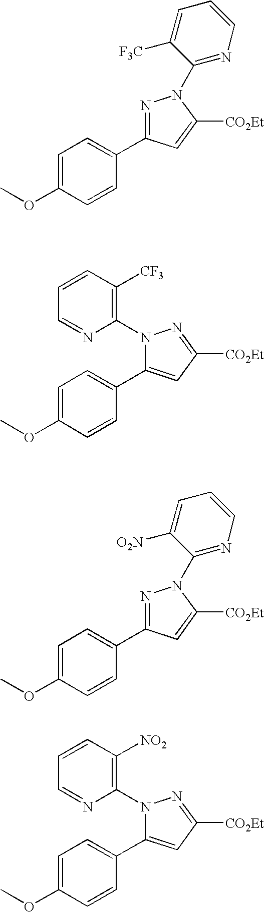

Compound 60: 5-(4-Methoxy-phenyl)-2-(3-nitro-pyridin-2-yl)-2H-pyrazole-3-carboxylic acid ethyl ester; and

Compound 61: 5-(4-Methoxy-phenyl)-1-(3-nitro-pyridin-2-yl)-1H-pyrazole-3-carboxylic acid ethyl ester: Prepared according to the same procedure as 5-(4-Methoxy-phenyl)-2-pyridin-2-yl-2H-pyrazole-3-carboxylic acid ethyl ester. For 60: [M+H]⁺ 369.1 ¹H NMR (CDCl₃) δ 8.74 (dd, J=2.0, 4.8 Hz, 1 H), 8.36 (dd, J=2.0, 8.0 Hz, 1 H), 7.59 (dd, J=4.8, 8.0 Hz, 1 H), 7.11-7.14 (m, 2 H), 7.02 (s, 1 H), 6.79-6.82 (m, 2 H), 4.45 (q, J=7.6 Hz, 2 H), 3.70 (s, 3 H), 1.40 (t, J =7.6 Hz, 3 H); for 61: ¹H NMR (CDCl₃) δ 8.81 (dd, J=2.0, 4.8 Hz, 1 H), 8.50 (dd, J=2.0, 8.0 Hz, 1 H), 7.76-7.81 (m, 3H), 7.62(dd, J=4.8, 8.0 Hz, 1 H), 6.92-6.96 (m, 2 H), 4.21 (q, J=7.6 Hz, 2 H), 3.80(s, 3 H), 1.35, J=7.6 Hz, 3 H):

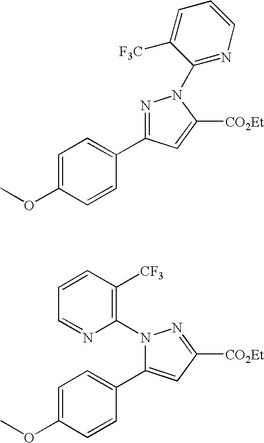

Compound 65: 5-Naphthalen-2-yl-2-pyridin-2-yl-2H-pyrazole-3-carboxylic acid ethyl ester; and

Compound 66: 5-Naphthalen-2-yl-1-pyridin-2-yl-1H-pyrazole-3-carboxylic acid ethyl ester: Prepared according to the same procedure as 5-(4-Methoxy-phenyl)-2-pyridin-2-yl-2H-pyrazole-3-carboxylic acid ethyl ester. For 65: [M+H]⁺ 344.2 for the 1,3-isomer ¹H NMR (CDCl₃) δ 8.33 (dd, J=1.8, 4.8 Hz, 1 H), 7.71-7.81 (m, 5 H), 7.63 (dt, J=1.8, 8.0 Hz), 7.46-7.53 (m, 2 H), 7.27 (ddd, J=1.2, 4.8, 7.6 Hz, 1 H), 7.23 (dd, J=1.8, 8.4Hz, 1 H), 7.13 (s, 3 H), 4.47 (q, J=7.6 Hz, 2 H), 3.48 (s, 3 H), 1.45 (t, J=7.6 Hz, 3 H); for 66: ¹H NMR (CDCl₃) δ 8.51 (dd, J=1.8, 4.8 Hz, 1 H), 8.33 (br s, 1 H), 8.04 (dd, J=1.8, 8.4 Hz, 1 H), 7.71-7.93 (m, 6 H), 7.46-7.51 (m, 2 H), 7.35 (s, 1 H), 4.35 (q, J=7.6 Hz, 2 H), 1.32 (t, J=7.6 Hz, 3 H):

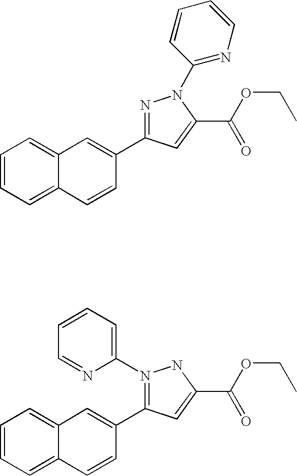

Compound 68: 5-Naphthalen-2-yl-2-(3-trifluoromethyl-pyridin-2-yl)-2H-pyrazole-3-carboxylic acid methyl ester; and

Compound 67: 5-Naphthalen-2-yl-1-(3-trifluoromethyl-pyridin-2-yl)-1H-pyrazole-3-carboxylic acid ethyl ester: Prepared according to the same procedure as 5-(4-Methoxy-phenyl)-2-pyridin-2-yl-2H-pyrazole-3-carboxylic acid ethyl ester. For 68: [M+H]⁺ 412.2 for 1,3-isomer and [M+H]⁺ 412.3 for 1,2-isomer. 1,3-isomer ¹H NMR (CDCl₃) δ 8.71 (dd, J=1.8, 4.8 Hz, 1 H), 8.13 (dd, J=2.2 8.0 Hz, 1 H), 7.66-7.79 (m, 5 H), 7.55 (ddd, J=1.2, 4.8, 8.0 Hz, 1 H), 7.42-7.50 (m, 2 H), 7.29 (dd, J=2.2, 8.8 Hz, 1 H), 7.18 (s, 1 H), 4.47 (q, J=7.6 Hz, 2 H), 1.44 (t, J=7.6 Hz, 3 H); for 67: ¹H NMR (CDCl₃) δ 8.81 (dd, J=1.8, 4.8 Hz, 1 H), 8.35 (br s, 1 H), 8.22 (dd, J=1.8, 8.4 Hz, 1 H), 8.00 (dd, J=1.8, 8.0 Hz, 1 H), 7.80-7.93 (m, 4 H), 7.64 (dd, J=4.8, 8.0 Hz, 1 H), 7.44-7.52 (m, 3 H), 4.24 (q, J=7.6 Hz, 2 H), 1.25 (t, J=7.6 Hz, 3 H):

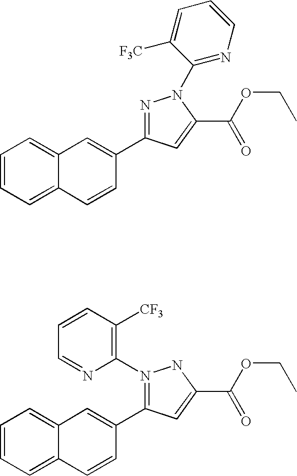

Compound 64: 5-Naphthalen-2-yl-2-(3-nitro-pyridin-2-yl)-2H-pyrazole-3-carboxylic acid methyl ester; and

Compound 69: 5-Naphthalen-2-yl-1-(6-nitro-pyridin-2-yl)-1H-pyrazole-3-carboxylic acid methyl ester: Prepared according to the same procedure as 5-(4-Methoxy-phenyl)-2-pyridin-2-yl-2H-pyrazole-3-carboxylic acid ethyl ester. For 64: [M+H]⁺ 389.2; ¹H NMR (CDCl₃) δ 8.85 (dd, J=1.5, 4.8 Hz, 1 H), 8.54 (dd, J=1.5, 8.1 Hz, 1 H), 8.31 (br s, 1 H), 7.98 (dd, J=1.5, 8.1 Hz, 1 H), 7.81-7.91 (m, 5 H), 7.68 (dd, J=4.8, 8.1 Hz, 1 H), 7.46-7.51 (m, 3 H), 4.29 (q, J=7.6 Hz, 2 H), 1.32 (t, J=7.6 Hz, 3 H); for 69: [M+H]⁺ 389.3:

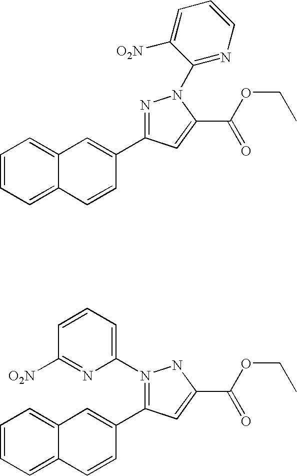

The following compounds were made by the procedure of Example 6—Step 2:

Compound 62: 5-(4-Methoxy-phenyl)-2-(3-trifluoromethyl-pyridin-2-yl)-2H-pyrazole-3-carboxylic acid: Prepared according to the same procedure used for 5-(4-Methoxy-phenyl)-2-pyridin-2-yl-2H-pyrazole-3-carboxylic acid. [M+H]⁺ 364.2:

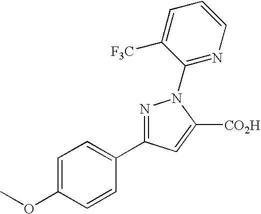

Compound 63: 5-Naphthalen-2-yl-1-(3-trifluoromethyl-pyridin-2-yl)-1 H-pyrazole-3-carboxylic acid: Prepared according to the same procedure used for 5-(4-Methoxy-phenyl)-2-pyridin-2-yl-2H-pyrazole-3-carboxylic acid. [M+H]⁺ 364.1:

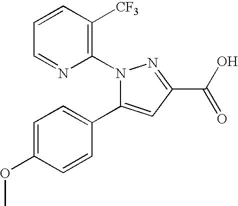

Compound 73: 5-Naphthalen-2-yl-2-pyridin-2-yl-2H-pyrazole-3-carboxylic acid: Prepared according to the same procedure used for 5-(4-Methoxy-phenyl)-2-pyridin-2-yl-2H-pyrazole-3-carboxylic acid. [M+H]⁺ 316.3:

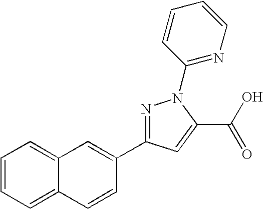

Compound 74: 5-Naphthalen-2-yl-1-pyridin-2-yl-1H-pyrazole-3-carboxylic acid: Prepared according to the same procedure used for 5-(4-Methoxy-phenyl)-2-pyridin-2-yl-2H-pyrazole-3-carboxylic acid. [M+H]⁺ 316.1:

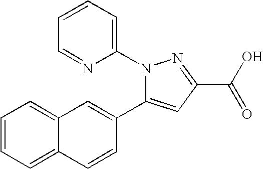

Compound 78: 5-Naphthalen-2-yl-2-(3-trifluoromethyl-pyridin-2-yl)-2H-pyrazole-3-carboxylic acid: Prepared according to the same procedure used for 5-(4-Methoxy-phenyl)-2-pyridin-2-yl-2H-pyrazole-3-carboxylic acid. [M+H]⁺ 384.3; 1H NMR (CD₃OD) δ 8.78 (dd, J=1.8, 4.8 Hz, 1 H), 8.37 (dd, J=1.8, 8.0 Hz, 1 H), 7.67-7.82 (m, 5 H), 7.43-7.51 (m, 2 H), 7.29 (dd, J=1.8, 8.4 Hz, 1 H), 7.22 (s, 1 H):

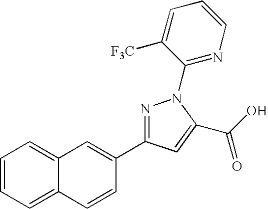

Compound 79: 5-Naphthalen-2-yl-1-(3-trifluoromethyl-pyridin-2-yl)-1H-pyrazole-3-carboxylic acid: [M+H]⁺ 384.2:

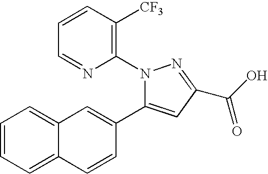

Compound 75: 5-Naphthalen-2-yl-1-(3-trifluoromethyl-pyridin-2-yl)-1H-pyrazole-3-carboxylic acid: Prepared according to the same procedure used for 5-(4-Methoxy-phenyl)-2-pyridin-2-yl-2H-pyrazole-3-carboxylic acid. [M+H]⁺ 361.3:

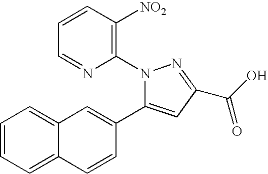

The following compounds were made according to the procedure of Example 6—Step 3: Compound 55: 2-(4-Chloro-phenyl)-5-(4-methoxy-phenyl)-2H-pyrazole-3-carboxylic acid propylamide: [M+H]⁺ 405.2:

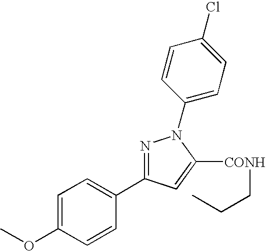

Compound 59: 5-(4-Methoxy-phenyl)-2-(3-trifluoromethyl-pyridin-2-yl)-2H-pyrazole-3-carboxylic acid propylamide: Prepared according to the same procedure as 5-(4-Methoxy-phenyl)-2-pyridin-2-yl-2H-pyrazole-3-carboxylic acid propylamide. [M+H]⁺ 405.2:

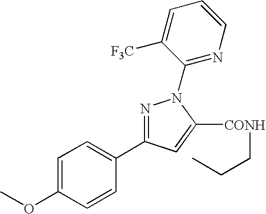

Compound 64: 5-(4-Methoxy-phenyl)-1-(3-trifluoromethyl-pyridin-2-yl)-1H-pyrazole-3-carboxylic acid propylamide: Prepared according to the same procedure as 5-(4-Methoxy-phenyl)-2-pyridin-2-yl-2H-pyrazole-3-carboxylic acid propylamide. [M+H]⁺ 405.2:

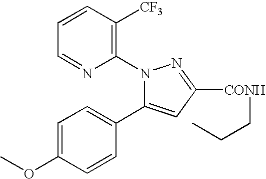

Compound 70: 5-(4-Methoxy-phenyl)-2-(3-nitro-pyridin-2-yl)-2H-pyrazole-3-carboxylic acid propylamide: [M+H]⁺ 382.2:

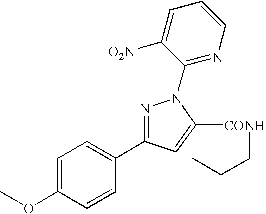

Compound 71: 5-Naphthalen-2-yl-2-pyridin-2-yl-2H-pyrazole-3-carboxylic acid propylamide: Prepared according to the same procedure as 5-(4-Methoxy-phenyl)-2-pyridin-2-yl-2H-pyrazole-3-carboxylic acid propylamide. [M+H]⁺ 357.3 ¹H NMR (CD₃OD) δ 8.33 (dd, J=1.8, 4.8 Hz, 1 H), 7.93 (td, J=1.8, 8.0 Hz, 1 H), 7.75-7.85 (m, 4 H), 7.61 (dt, J=1.2, 8.4 Hz, 1 H), 7.45-7.53 (m, 2 H), 7.41 (ddd, J=1.2, 4.8, 7.8 Hz, 1 H), 7.25 (dd, J=1.8, 8.4 Hz, 1 H), 7.09 (s, 1 H), 3.39 (t, J=7.4 Hz, 2 H), 1.63-1.73 (m, 2 H), 1.01 (t, J=7.6 Hz, 3 H):

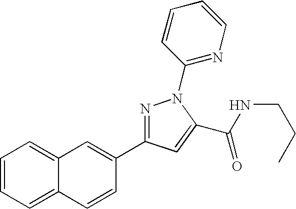

Compound 72: 5-Naphthalen-2-yl-1-pyridin-2-yl-1H-pyrazole-3-carboxylic acid propylamide: Prepared according to the same procedure as 5-(4-Methoxy-phenyl)-2-pyridin-2-yl-2H-pyrazole-3-carboxylic acid propylamide. [M+H]⁺ 357.3:

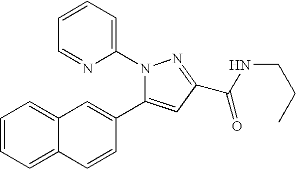

Compound 76: 5-Naphthalen-2-yl-2-(3-trifluoromethyl-pyridin-2-yl)-2H-pyrazole-3-carboxylic acid propylamide (76): Prepared according to the same procedure as 5-(4-Methoxy-phenyl)-2-pyridin-2-yl-2H-pyrazole-3-carboxylic acid propylamide. [M+H]⁺ 425.3; ¹H NMR (CD₃OD) δ 8.74 (dd, J=1.8, 4.4 Hz, 1 H), 8.39 (dd, J=1.8, 7.4 Hz, 1 H), 7.73-7.83 (m, 3 H), 7.67-7.73 (m, 2 H), 7.43-7.51 (m, 2 H), 7.28 (dd, J=1.8, 9.2 Hz, 1 H), 7.16 (s, 1 H), 3.38 (t, J=7.3 Hz, 2 H), 1.61-1.70 (m, 2 H), 0.97 (t, J=8.1 Hz, 3 H):

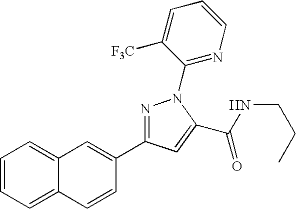

Compound 77: 5-Naphthalen-2-yl-1-(3-trifluoromethyl-pyridin-2-yl)-1H-pyrazole-3-carboxylic acid propylamide (77): Prepared according to the same procedure as 5-(4-Methoxy-phenyl)-2-pyridin-2-yl-2H-pyrazole-3-carboxylic acid propylamide. [M+H]⁺ 425.3; ¹H NMR (CD₃OD) δ 8.78 (dd, J=1.8, 4.4 Hz, 1 H), 8.66-8.72 (m, 1 H), 8.39 (dd, J=1.8, 7.4 Hz, 1 H), 8.32 (br s, 1 H), 7.99 (dd, J=1.8, 8.0 Hz, 1 H), 7.76-7.90 (m, 5 H), 7.44-7.54 (m, 3 H), 3.23 (q, J=7.6 Hz, 2 H), 1.55-1.66 (m, 2 H), 0.98 (t, J=7.4 Hz, 3 H): 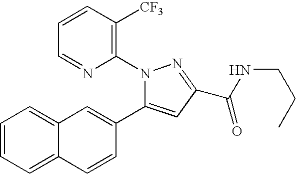
Isoxazoles

Scheme 5 shows a general procedure to prepare the 1,3-disubstituted isoxazoles.

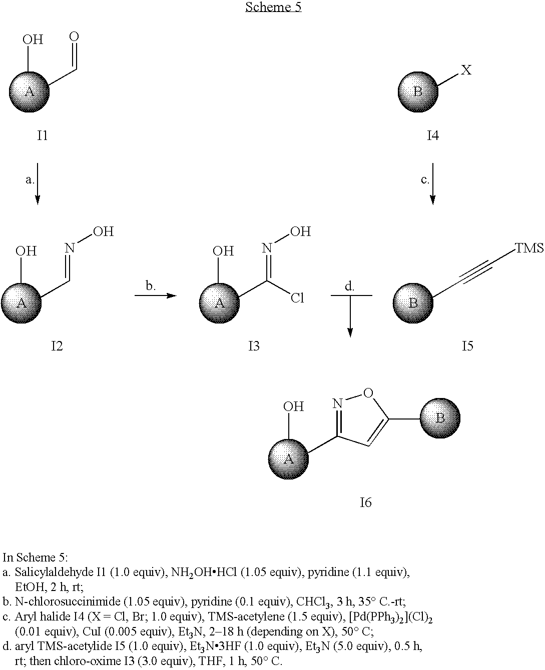

In a typical synthetic route, salicylaldehyde I1 is reacted with hydroxylamine hydrochloride in ethanol to furnish the corresponding aldoxime I2, and crystallized from the reaction solution as a single stereoisomer by the addition of water. The aldoxime is then reacted with 1 equivalent of N-chlorosuccinimide at ambient temperature (with only electron deficient systems requiring any heat to initiate the reaction) to afford chloroaldoxime I3. Consumption of the N-chlorosuccinimide is conveniently monitored with starch paper. Electron rich aryl systems often exhibited ring chlorination at a rate that was competitive with chloroaldoxime formation. In these cases, an excess of the crude chloroaldoxime was used for the formation of the isoxazole. For the other component of the isoxazole, aryl or pyridyl halides I4 were reacted with trimethylsilyl acetylene under Sonagashira coupling conditions; using a catalytic amount of bis-triphenylphosphine palladium dichloride and copper (I) iodide in triethylamine as a solvent. In the case of aryl or pyridyl bromides, the reactions were sufficiently rapid to not require any heating, whereas the aryl or pyridyl chlorides required heating, prolonged reaction times and higher catalyst loading. The reactions were diluted with diethyl ether, filtered and concentrated, with the crude oils being purified by chromatography. The trimethylsilyl aryl or pyridyl acetylides were then dissolved in triethylamine and triethylamine trihydrofluoride was added. When TLC analysis indicated complete conversion of the trimethylsilylalkyne to the free alkyne, a solution of the desired chloroaldoxime in THF was added and the resulting solution heated to 50° C. for 2 hours. The resulting solution was concentrated and purified by chromatography to yield the desired compound I6.

### EXAMPLE 7

Compound 125: 4-Methoxy-2-(5-pyridin-2-yl-isoxazol-3-yl)-phenol

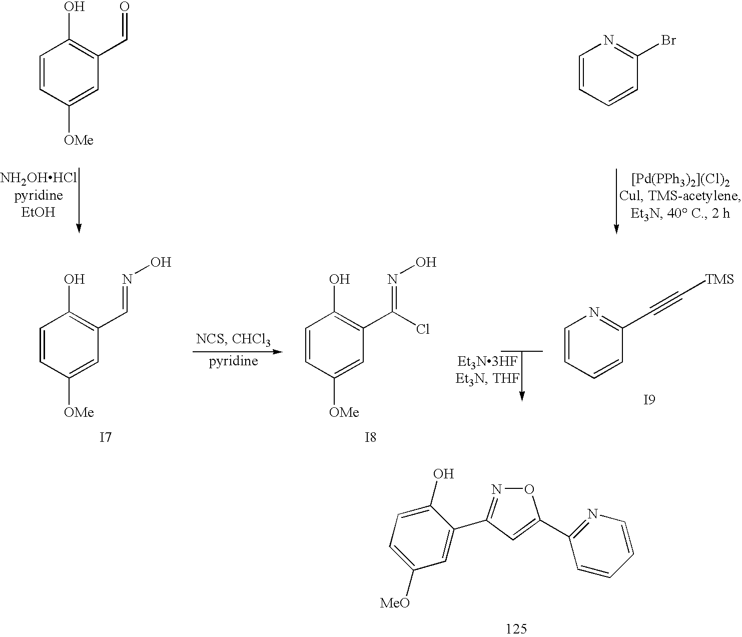

Step 1: Preparation of 2-Hydroxy-5-methoxy-benzaldehyde oxime (I7).

To a stirred solution of 2-hydroxy-5-methoxy-benzaldehyde (0.913 g, 6.0 mmol) in ethanol (10 mL) was added hydroxylamine hydrochloride (1.05 equiv, 0.459 g, 6.3 mmol) and pyridine (1.05 equiv, 500 μL, 6.35 mmol). The solution was briefly heated to 35° C. and after 1 hour, distilled water (3 mL) was added with heating. The crystals that formed upon cooling were filtered off, affording the title compound I7 as a white solid (0.982 g, 98%). [M+H]⁺ 168.0; ¹H (d⁶-DMSO) δ 11.25 (s, 1 H), 9.55 (s, 1 H), 8.25 (s, 1 H), 7.03 (d, J=3.2 Hz, 1 H), 6.77-6.83 (m, 2 H), 3.67 (s, 3 H).

Step 2: Preparation of 2-Hydroxy-5-methoxy-benzaldehyde chloroxime (I8).

To a stirred solution of 2-hydroxy-5-methoxy-benzaldehyde oxime I7 (0.881 g, 5.30 mmol) in dry chloroform (10 mL) was added pyridine (20 μL) and N-chlorosuccinimide (1.1 equiv, 0.774 g, 5.83 mmol). The resulting solution was briefly heated to 35° C. and stirred at room temperature for 2 h at which point it was analyzed by HPLC-MS which indicated a small amount of dichlorinated product was present. The reaction was diluted with CH₂Cl₂ (20 mL) and washed with water (2×40 mL), brine (2×40 mL), dried (MgSO₄), and concentrated to yield a deep orange solid that was used without further purification. [M+H]⁺ 202.2.

Step 3: Preparation of 2-Trimethylsilanylethynyl-pyridine I9.

To a stirred solution of 2-bromopyridine (3.95 g, 25 mmol) in triethylamine (20 mL) was added bis-triphenylphosphine palladium dichloride ([Pd(PPh₃)₂](Cl)₂, 0.18 g, 0.01 equiv), copper (I) iodide (0.020 g, 0.005 equiv) and trimethylsilyl-acetylene (1.25 equiv, 4.30 miL, 31 mmol). The resulting suspension underwent a rapid color change (yellow→dark brown) and was heated to 50° C. for 2 h at which point TLC (20% EtOAc in hexanes) indicated complete conversion of the starting material. The reaction was diluted with diethyl ether (50 mL), filtered through a pad of celite, concentrated and the resulting syrup was purified by chromatography (0-20% EtOAc in hexanes) to yield the pyridyl-alkyne I9 as a golden liquid (4.20 g, 96%). ¹H (CDCl₃) δ 8.55 (bd, J=4.8 Hz, 1 H), 7.62 (td, J=2.0, 7.6 Hz, 1 H), 7.44 (dt, J=1.2, 7.6 Hz, 1 H), 7.21 (ddd, J=1.2, 4.8, 7.8 Hz, 1 H), 0.28 (s, 9 H)

Step 4

Compound 125: 4-Methoxy-2-(5-pyridin-2-yl-isoxazol-3-yl)-phenol

To a cooled (0° C.), stirred solution of 2-trimethylsilanylethynyl-pyridine I9 (0.263 g, 1.5 mmol) in THF (2 mL) and triethylamine (2 mL) was added triethylamine trihydrofluoride (0.250 mL, 1 equiv). Once TLC analysis indicated conversion of the trimethylsilyl-acetylide to a more polar spot (20% EtOAc in hexanes, UV and KMnO₄), a solution of 2-hydroxy-5-methoxy-benzaldehyde chloroxime (I8) in tetrahydrofuran (2 mL) was treated with triethylamine (1 mL) and stirred until a precipitate was observed. The suspension was then transferred to the solution containing the pyridyl-alkyne with stirring and the mixture was heated to 50° C. for 1 hour. The resulting mixture was concentrated and purified by chromatography to yield the title compound as a slightly yellow solid. ¹H (CDCl₃) δ 9.39 (s, 1 H), 8.57-8.59 (m, 1 H), 8.17 (d, J=2.8 Hz, 1 H), 7.67 (dt, J=1.6, 7.6 Hz, 1 H), 7.49 (bd, J=7.6 Hz, 1 H), 7.26-7.30 (m, 2 H), 6.85-6.91 (m, 2 H), 6.75 (dd, J=3.2, 15.2 Hz, 1 H), 3.78 (s, 3 H).

The following compounds were prepared according to the procedure of Example 7—step 1:

5-bromo-2-hydroxy-benzaldehyde oxime (I10): Prepared according to the same procedure used for 2-hydroxy-5-methoxy-benzaldehyde oxime. ¹H NMR (d⁶-DMSO) δ 11.43 (s, 1 H), 10.26 (s, 1 H), 8.24 (s, 1 H), 7.61 (d, J=2.8 Hz, 1 H), 7.34 (dd, J=2.8, 8.4 Hz, 1 H), 6.83 (d, J=8.4 Hz, 1 H):

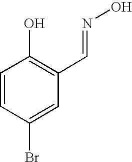

3,5-Dichloro-2-hydroxy-benzaldehyde oxime (I11): Prepared according to the same procedure used for 2-hydroxy-5-methoxy-benzaldehyde oxime. ¹H NMR (d⁶-DMSO) δ 11.87 (s, 1 H), 10.81 (s, 1 H), 8.36 (s, 1 H), 7.54 (d, J=2.4 Hz, 1 H), 7.50 (d, J=2.4 Hz, 1 H):

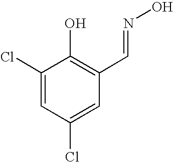

5-chloro-2-hydroxy-benzaldehyde oxime (I12): Prepared according to the same procedure used for 2-hydroxy-5-methoxy-benzaldehyde oxime. ¹H NMR (CDCl₃) δ 9.71 (s, 1 H), 8.15 (s, 1 H), 7.39 (s, 1 H), 7.23 (dd, J=2.8, 8.8 Hz, 1 H), 7.15 (d, J=2.8 Hz, 1 H), 6.91 (d, J=8.8 Hz, 1 H):

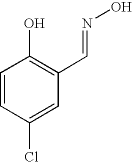

T3 the following compounds were prepared according to the procedure of Example 7—step 2: 3-Methyl-2-trimethylsilanylethynyl-pyridine I(13): Prepared according to the same procedure used for 2-trimthylsilanylethynyl-pyridine. 1H NMR (CDCl₃) δ 8.39 (dd, J=1.6, 4.8 Hz, 1 H), 7.49 (app d, J=7.6 Hz, 1 H), 7.12 (dd, J=4.8, 7.6 Hz, 1 H), 2.44 (s, 3 H), 0.29 (s, 9 H):

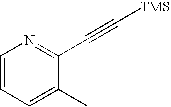

3-Trifluoromethyl-2-trimethylsilanylethynyl-pyridine (I14): Prepared according to the same procedure used for 2-trimthylsilanylethynyl-pyridine. ¹H NMR (CDCl₃) δ 8.79 (dd, J=1.6, 4.8 Hz, 1 H), 8.32 (app d, J=7.6 Hz, 1 H), 7.43 (dd, J=4.8, 7.6 Hz, 1 H), 0.29 (s, 9 H):

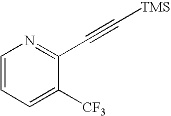

3-Nitro-2-trimethylsilanylethynyl-pyridine (I15): Prepared according to the same procedure used for 2-trimthylsilanylethynyl-pyridine. [M+HJ+222.3; ¹H NMR (CDCl₃) δ 8.79 (dd, J=1.6, 4.8 Hz, 1 H), 8.31 (dd, J=1.6, 8.8 Hz, 1 H), 7.43 (dd, J=4.8, 8.8 Hz, 1 H), 0.32 (s, 9 H):

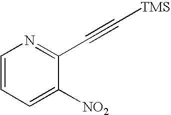

The following compounds were prepared according to the procedure of Example 7—step 4:

Compound 127: 2,4-Dichloro-6-(5-pyridin-2-yl-isoxazol-3-yl)-phenol: Prepared according to the same procedure used for 4-methoxy-2-(5-pyridin-2-yl-isoxazol-3-yl)-phenol. [M+H]⁺ 307.2; ¹H NMR (CDCl₃) δ 9.98 (s, 1 H), 8.74 (app d, J=4.8 Hz, 1 H), 7.96 (app d, J=8.0 Hz, 1 H), 7.88 (td, J=2.0, 8.0 Hz, 1 H), 7.52 (d, J=2.0 Hz, 1 H), 7.46 (d, J=2.4 Hz, 1 H), 7.42 (ddd, J=1.2, 4.8, 7.2 Hz, 1 H), 7.34 (s, 1 H):

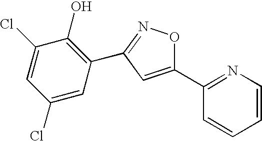

Compound 126: 2-chloro-6-(5-pyridin-2-yl-isoxazol-3-yl)-phenol: Prepared according to the same procedure used for 4-methoxy-2-(5-pyridin-2-yl-isoxazol-3-yl)-phenol. ¹H NMR (CDCl₃) δ 10.25 (s, 1 H), 8.79 (d, J=4.8 Hz, 1 H), 7.99 (d, J=8.0 Hz, 1 H), 7.94 (td, J=1.6, 7.6 Hz, 1 H), 7.55 (d, J=2.8 Hz, 1 H), 7.46 (ddd, J=1.6, 2.8, 5.2 Hz, 1 H), 7.31 (dd, J=2.4, 8.8 Hz, 1 H), 7.04 (d, J=8.8 Hz, 1 H):

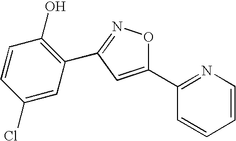

Compound 129: 4-Methoxy-2-[5-(3-trifluoromethyl-pyridin-2-yl)-isoxazol-3-yl]-phenol: Prepared according to the same procedure used for 4-methoxy-2-(5-pyridin-2-yl-isoxazol-3-yl)-phenol. [M+H]⁺ 337.3; ¹H (CDCl₃) δ 9.00 (s, 1 H), 8.93 (d, J=4.8 Hz, 1 H), 8.20 (d, J=7.6 Hz, 1 H), 7.58 (dd, J=4.8, 8.8 Hz, 1 H), 7.25 (s, 1 H), 7.08 (d, J=2.8 Hz, 1 H), 7.05 (d, J=8.8 Hz, 1 H), 6.98 (dd, J=2.8, 7.6 Hz, 1 H):

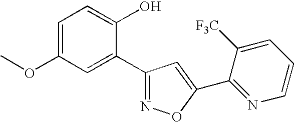

Compound 128: 4-Bromo-2-[5-(3-trifluoromethyl-pyridin-2-yl)-isoxazol-3-yl]-phenol: Prepared according to the same procedure used for 4-methoxy-2-(5-pyridin-2-yl-isoxazol-3-yl)-phenol.¹H (CDCl₃) δ 9.44(s, 1 H), 8.93 (d, J=3.6 Hz, 1 H), 8.20 (dd, J=1.2, 8.0 Hz, 1 H), 7.69 (d, J=2.0 Hz, 1 H), 7.59 (dd, J=4.8, 8.0 Hz, 1 H), 7.44 (dd, J=2.0, 8.8 Hz, 1 H), 7.24-7.27 (m, 2H),7.01 (d,J=8.8Hz, 1 H):

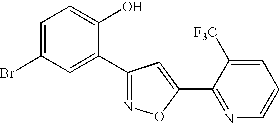

Compound 132: 4-Methoxy-2-[5-(3-methyl-pyridin-2-yl)-isoxazol-3-yl]-phenol: Prepared according to the same procedure used for 4-methoxy-2-(5-pyridin-2-yl-isoxazol-3-yl)-phenol. [M+H]⁺ 283.2:

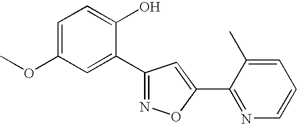

Compound 131: 4-Bromo-2-[5-(3-methyl-pyridin-2-yl)-isoxazol-3-yl]-phenol: Prepared according to the same procedure used for 4-methoxy-2-(5-pyridin-2-yl-isoxazol-3-yl)-phenol. [M+H]⁺ 331.3, 333.3 (Br^{79, 81}):

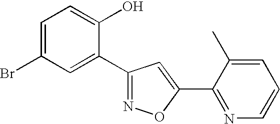

Compound 130: 4-Bromo-2-(5-pyridin-2-yl-isoxazol-3-yl)-phenol: Prepared according to the same procedure used for 4-methoxy-2-(5-pyridin-2-yl-isoxazol-3-yl)-phenol. [M+H]+317.0, 319.0 (Br^{79,81}) ¹H NMR (CDCl₃) δ 9.50 (s, 1 H), 8.73 (d, J=4.8 Hz, 1 H), 7.95 (d, J=8.0 Hz, 1 H), 7.87 (td, J=1.6, 8.0 Hz, 1 H), 7.71 (d, J=2.8 Hz, 1 H), 7.38-7.45 (m, 2 H), 7.33 (s, 1 H), 6.99 (d, J-=8.0 Hz, 1 H):

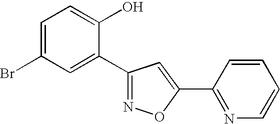

Compound 116: 4-Nitro-2-(5-pyridin-2-yl-isoxazol-3-yl)-phenol: Prepared according to the same procedure used for 4-methoxy-2-(5-pyridin-2-yl-isoxazol-3-yl)-phenol. [M+H]+284.1:

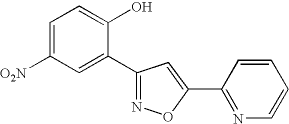

Compound 124: 2-Methoxy-6-(5-pyridin-2-yl-isoxazol-3-yl)-phenol: Prepared according to the same procedure used for 4-methoxy-2-(5-pyridin-2-yl-isoxazol-3-yl)-phenol. [M+H]+269.3; ¹H NMR (CDCl₃) δ 9.07 (s, 1 H), 8.72 (d, J=4.4 Hz, 1 H), 7.97 ( dd, J=0.8, 8.0 Hz, 1 H), 7.88 (tt, J=1.6, 8.4 Hz, 1 H), 7.38-7.42 (m, 1 H), 7.33 (d, J=2.0 Hz, 1 H), 7.02-7.10 (m, 2 H), 6.96 (dd, J=2.0, 8.8 Hz, 1 H), 4.01 (s, 3 H):

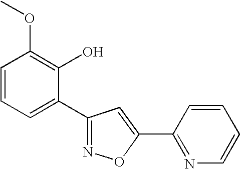

Compound 123: 2-(5-Pyridin-2-yl-isoxazol-3-yl)-phenol: Prepared according to the same 15 procedure used for 4-methoxy-2-(5-pyridin-2-yl-isoxazol-3-yl)-phenol. ¹H NMR (CDCl₃) δ 9.47 (s, 1 H), 8.73 (dd, J=1.2, 4.8 Hz, 1 H), 7.97 (d, J=6.4 Hz, 1 H), 7.88 (td, J=1.6, 8.0 Hz, 1 H), 7.61 (dd, J=1.2, 8.0 Hz, 1 H), 7.35-7.42 (m, 2 H), 7.10 (d, J=8.0 Hz, 1 H), 7.00 (td, J=1.2, 7.6 Hz, 1 H): 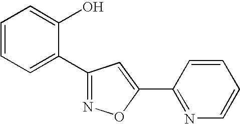
1.2,4-Oxadiazoles

Scheme 6 shows a general procedure to prepare the 3,5-disubstituted 1,2,4-oxadiazoles.

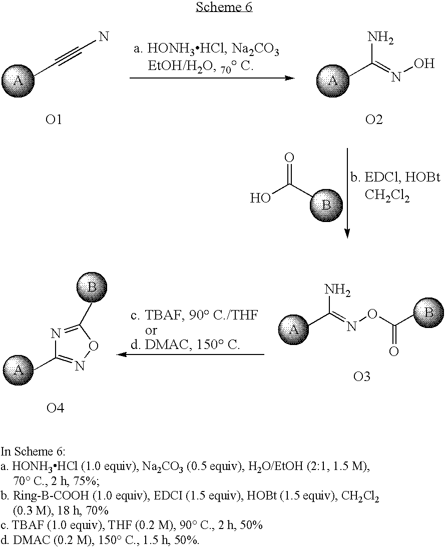

In a typical synthetic route, the Aryl nitrile O1 is first converted to the N-hydroxycarboxamidine as shown in scheme O2. A coupling reaction with a carboxylic acid furmished the O-Aroyl-carboxamidoxime O3. The desired 3,5-disubstituted 1,2,4-oxadiazoles O4 were then obtained through intramolecular cyclization of the above intermediate, followed by chromatographic separation from impurities.

A typical synthetic route for analogs in which A (see scheme 6) requires a pyridinyl-2-carbonitrile moiety begins with a pyridinyl moiety as shown in scheme 7. The pyridinyl moiety O5 is first converted to the corresponding N-oxide O6 by heating with hydrogen peroxide in acetic acid at reflux. The above intermediate is then regioselectively cyanated at the ortho position with trimethylsilyl cyanide. Purification of the nitrile O7 product is then carried out by chromatographic separation.

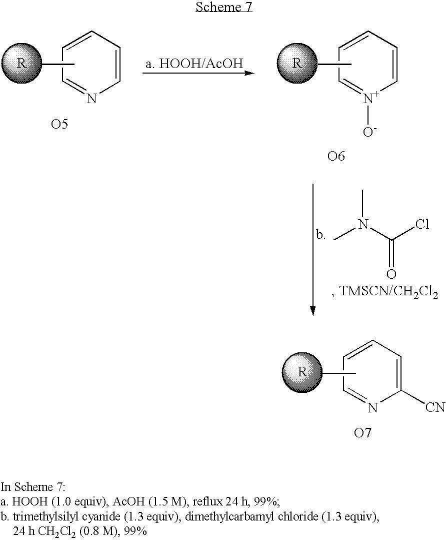

### EXAMPLE 8

Compound 220: 2-[5-(3-Methoxy-phenyl)-[1,2,41oxadiazol-3-yl]-5-trifluoromethyl-Pyridine (220)

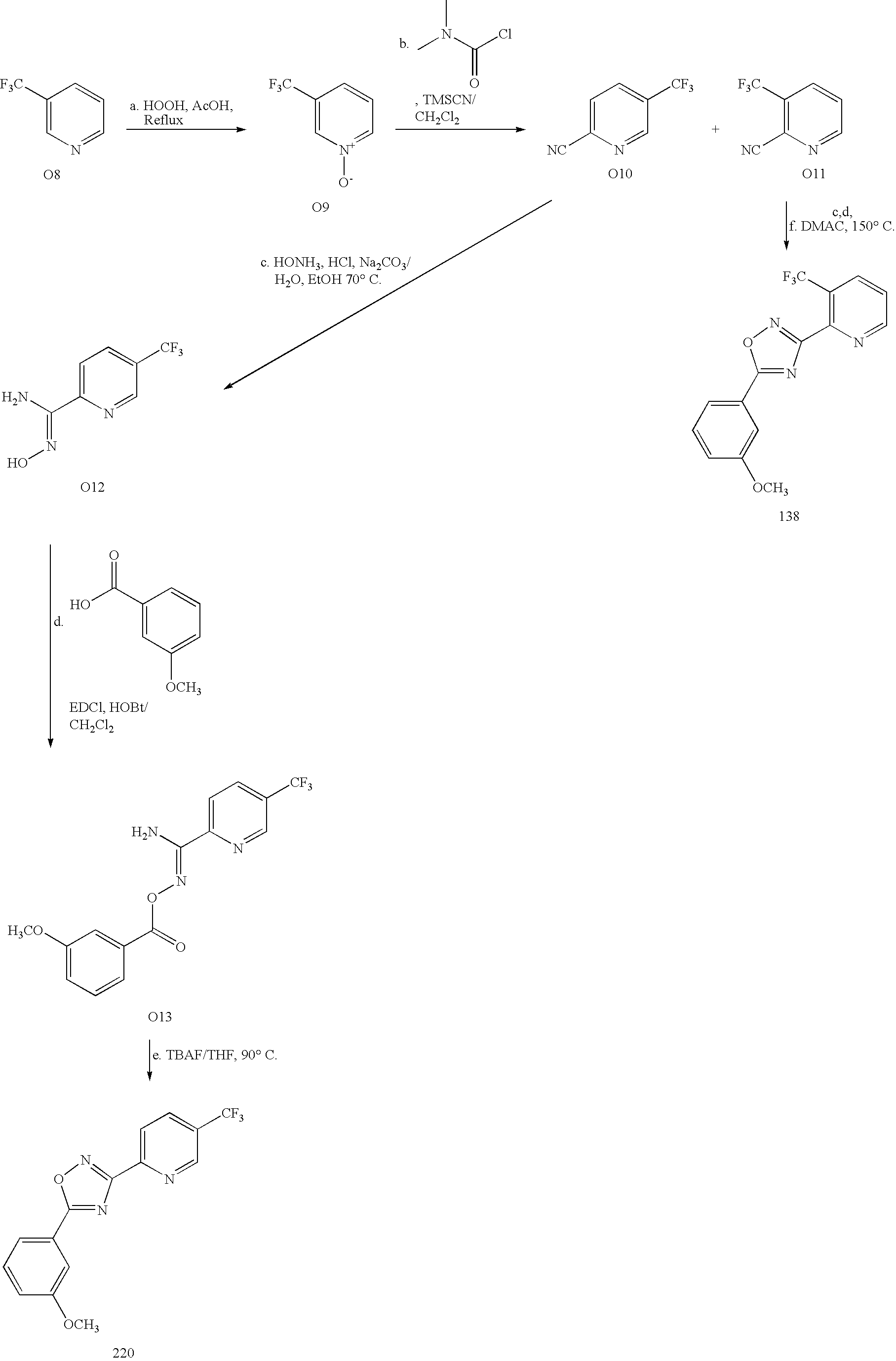

Step 1: Preparation of 3-Trifluoromethyl-pyridine 1-oxide O9.

To a solution of O8 3-trifluoromethylpyridine (5 g, 34 mmoles) in acetic acid was added 20% hydrogen peroxide in water (1.1 equiv, 5.8 mL, 39 numoles). A condenser was attached and the reaction mixture was refluxed for 24 h with stirring. After cooling to ambient temperature, the solvent was removed under reduced pressure. The residue was purified by column chromatography to produce 5.5 g of N-Oxide O9 as a yellow oil in 99% yield; [M+H]⁺ 164.3; R_{f}: 0.60 (10% MeOH/CH₂Cl₂); ¹H (CDCl₃) δ 8.53 (s, 1H), 8.42 (d, J=6.4Hz, 1H), 7.52 (d, J=8.4Hz, 1H), 7.45 (dd, J=6.4, 8 Hz, 1H).

Step 2: Preparation of 3-Trifluoromethyl-pyridine-2-carbonitrile O10 and 5-Trifluoromethyl-pyridine-2-carbonitrile O11.

To a solution of O9 N-oxide (5.5 g, 34 nmroles) in CH₂Cl₂ (40 mL) under nitrogen was added trimethylsilylcyanide (1.3 equiv, 5.8 mL, 44 mmoles) and stirred for 10 m. To the mixture, dimethylcarbamyl chloride (1.3 equiv, 4.0 mL, 44 mmoles) was added dropwise and stirred at ambient temperature for 24 h. The mixture was partitioned between dichloromethane and 5% NaHCO₃ in water, then brine. The organic phase was separated, dried with magnesium sulfate. The final products were obtained by chromatographic separation of the isomers to yield: O10 5-trifluoromethyl-pyridine-2-carbonitrile (3.3 g, 58% yield) was a clear oil; R_{f}: 0.59 (50% EtOAc/Hex); ¹H (CDCl₃) δ 8.93 (d, J=1.6 Hz, 1H), 8.22 (dd, J=8.0, 2.0 Hz, 1H), 7.86 (d, J=8.4 Hz, 1H); O11 3-trifluoromethyl-pyridine-2-carbonitrile (2.4 g, 41% yield) was a clear oil; R_{f}: 0.52 (50% EtOAc/Hex); ¹H (CDCl₃) δ 8.90 (d, J=4.4 Hz, 1H), 8.13 (dd, J=8.0, 0.8 Hz, 1H), 7.71 (ddd, J=8, 4.8, 0.8 Hz, ).

Step 3: Preparation of O12 N-Hydroxy-3-trifluoromethyl-pyridine-2-carboxamidine.

Hydroxylamine hydrochloride (1.03 equiv, 832 mg, 12.0 mmoles) was dissolved in 8 mL of water. Sodium Carbonate (0.5 equiv, 615 mg, 5.8 mmoles) was added to the solution at room temperature then warmed to 70° C. with stirring for 5 minutes. A solution of O10 5-trifluoromethyl-pyridine-2-carbonitrile (2.0 g, 11.6 mmols) dissolved in 6 mL of Ethanol was then added to the solution above. The reaction stirred for 3 h at 70° C. The reaction was cooled to ambient temperature and concentrated in vacuo. The white solid residue was then partitioned between ethyl acetate and water. The organic phase was separated, dried with magnesium sulfate, and dried in vacuo to yield 1.54 g of the desired product as a white powder in 65% yield. [M+H]⁺ 206.1; ¹H (CDCl₃) δ 8.91 (s, 1H), 8.16 (d, J=8.4Hz, 1H), 8.02 (d, J=8.4 Hz, 1H)., 5.90 (b, 3H).

Step 4: Preparation of O13 3-Methoxy-O-benzoyl-(5-trifluoromethyl-pyridine-2-carboxamidoxime) ester.

N-Hydroxy-5-trifluoromethyl-pyridine-2-carboxamidine O12 (60 mg, 0.29 mmoles) was dissolved in 2 ml dichloromethane. 3-methoxybenzoic acid (1.0 equiv., 50 mg, 0.29 rumoles), EDCI (1.5 equiv, 83 mg, 0.43 mmoles), and HOBt (1.5 equiv,59 mg, 0.43 mmoles) were then added, and the mixture was stirred at ambient temperature for 18 h. The mixture was then diluted in dichloromethane and partitioned with 5% sodium bicarbonate in water. The organic layer was then separated and dried over magnesium sulfate, and crude 3-Methoxy-O-benzoyl-(5-trifluoromethyl-pyridine-2-carboxamidoxime) ester O13 was isolated without further purification as 92 mg of crude white foam. [M+H]⁺ 340.2

Step 5

Compound 220: 2-[5-(3-Methoxy-phenyl)-[1,2,4]oxadiazol-3-yl]-5-trifluoromethyl-pyridine.

Crude O13 3-Methoxy-O-benzoyl-(5-trifluoromethyl-pyridine-2-carboxamidoxime) ester (92 mg) was dissolved in tetrahyrofuran (2 mL). Tetrabutylammoniumfluoride (290 □L, 1.0 N in THF) was added, and the reaction stirred in a sealed vial at 90° C. for 1.5 hours. After cooling to ambient temperature, the product was purified by chromatographic separation to yield 4.2 mg of a white powder in 5% yield.; R_{f}: 0.45 (10% MeOH/CH₂Cl₂); [M+H]⁺ 322.2; ¹H (CDCl₃) □ 9.09 (p, J=0.8 Hz, 1H), 8.37 (d, J=8.0, 1H), 8.14(dt, J=8.8, 2.8 Hz, 1H) , 7.87 (dt, J=8.0, 1.6 Hz, 1H), 7.78 (dd, J=8.0, 2.4, 1.6 Hz, 1H), 7.48 (t, J=8.0 Hz, 1H), 7.18 (ddd, J=8.4, 2.8, 1.2 Hz, 1H), 3.92 (s, 3H).

### EXAMPLE 9

Compound 138: 2-[5-(3-Methoxy-phenyl)-[1,2,4]oxadiazol-3-yl]-pyridine (according to the procedure of scheme 8 above)

Step 1: Preparation of O14 N-Hydroxy-3-trifluoromethyl-pyridine-2-carboxamidine.

O11 3-trifluoromethyl-pyridine-2-carbonitrile (1.8 g, 10.5 mmoles) , was subjected to conditions outlined in Example 8, step 3; to produce 1.10 g of a white powder in 52% yield. [M+H]⁺ 206.1; ¹H (CDCl₃) δ 8.68 (d, J=4.8 Hz, 1H), 8.00 (d, J=8.4Hz, 1H), 7.39 (dd, J=8.0, 4.8 Hz, 1H), 5.50 (b, 3H):

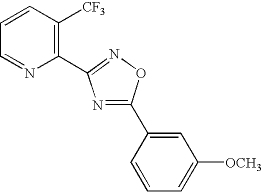

Step 2: Preparation of O15 3-Methoxy-O-benzoyl-(3-trifluoromethyl-pyridine-2-carboxamidoxime) ester.

O14 N-Hydroxy-3-trifluoromethyl-pyridine-2-carboxamidine (100 mg, 0.49 mmoles) was dissolved in 2 ml dichloromethane. 3-methoxybenzoic acid (1.0 equiv., 75 mg, 0.49 inmoles), EDCI (1.5 equiv, 141 mg, 0.73 mmoles), and HOBt (1.5 equiv,99 mg, 0.73 mmoles) were then added, and the mixture was stirred at ambient temperature for 18 h. The mixture was then diluted in dichloromethane and partitioned with 5% sodium bicarbonate in water. The organic layer was then separated and dried over magnesium sulfate, and purified by column chromatography to produce 100 mg of the O-aroyl-carboxamidoxime ester O15 as a white powder in 57% yield. [M+H]⁺ 340.2; ¹H (CDCl₃) δ 8.86 (d, J=0.4 Hz, 1H), 8.42(d, J=8.0 Hz, 1H), 8.02 (d, J=8.4, 2.4 Hz, 1H), 7.69 (dt, J=7.6, 1.2 Hz, 1H), 7.62 (dd, J=2.8,1.6 Hz, 1H), 7.40 (t, J=8 Hz, 1H), 7.15 (ddd, J=8.0, 2,4, 0.4, 1H), 3.90 (s, 3H):

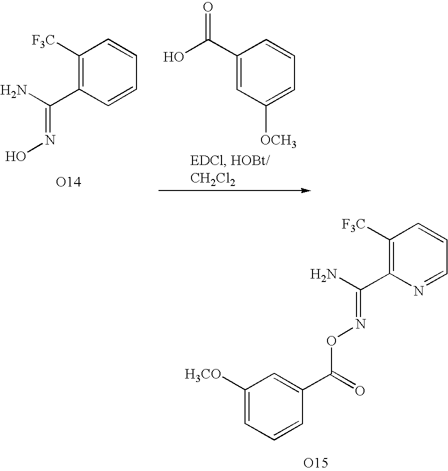

Step 3

Compound 138: 2-[5-(3-Methoxy-phenyl)-[1,2,4]oxadiazol-3-yl]-3-trifluoromethyl-pyridine.

O15 3-Methoxy-O-benzoyl-(3-trifluoromethyl-pyridine-2-carboxamidoxime) ester (100 mg, 0.29 mmoles) was dissolved in N,N-dimethylacetamide (2 mL), and stirred at 150° C. for 1.5 hours. The desired 2-[5-(3-Methoxy-phenyl)-[1,2,4]oxadiazol-3-yl]-3-trifluoromethyl-pyridine 138 was then purified by HPLC to yield 3 mg of a white powder in 3% yield. [M+H]⁺ 322.2:

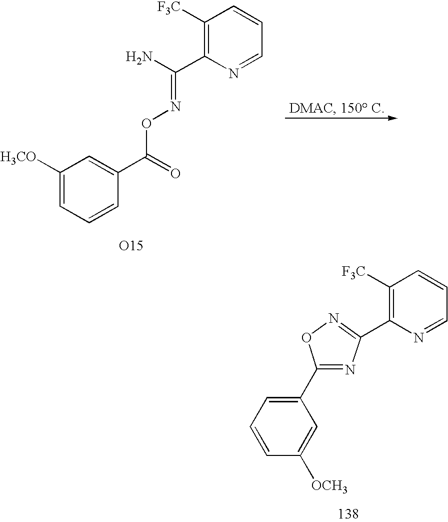

The following compounds were synthesized according to the procedure of Example 8.

Compound 153: 2-(5-Naphthalen-1-yl-[1,2,4]oxadiazol-3-yl)-3-trifluoromethyl-pyridine: 1-naphtoic acid and O14 N-Hydroxy-3-trifluoromethyl-pyridine-2-carboxamidine were subjected to protocol described above to yield 16mg of a brown oily solid in 22% yield. [M+H]⁺ 342.2; ¹H (CDCl₃) δ 9.21 (dd, J=8.4, 0.8 Hz, 1H), 9.01 (dd, J=8.0, 0.8 1H), 8.40 (dd, J=7.2, 1.2 Hz, 1H) , 8.24 (dd, J=8.4, 1.2 Hz, 1H), 8.12 (d, J=8.4 Hz, 1H), 7.95 (d, J=7.2 Hz, 1H), 7.66(m, 5H):

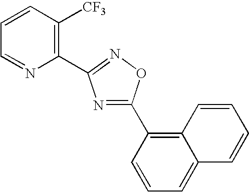

Compound 207: 2-Methoxy-6-[3-(3-trifluoromethyl-pyridin-2-yl)-[1,2,4]oxadiazol-5-yl]-phenol: 3-methoxysalicylic acid (82 mg, 0.49 mmoles) and O14 N-Hydroxy-3-trifluoromethyl-pyridine-2-carboxamidine (100 mg, 0.49 mmoles) were subjected to protocol described above to yield 30 mg of white powder in 18% yield. R_{f}: 0.52 (10% MeOH/CH₂Cl₂); [M+H]⁺ 338.2:

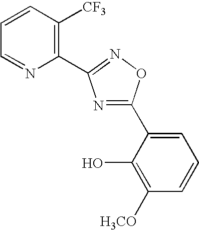

Compound 208: 4-Methoxy-2-[3-(3-trifluoromethyl-pyridin-2-yl)-[1,2,4]oxadiazol-5-yl]-phenol: 5-methoxysalicylic acid (82 mg, 0.49 mmoles) and O14 N-Hydroxy-3-trifluoromethyl-pyridine-2-carboxamidine (100 mg, 0.49 mmoles) were subjected to protocol described above to yield 22 mg of white powder in 13% yield. [M+H]⁺ 338.2; ¹H (CDCl₃) δ 8.98 (dd, J=4.4, 0.8 Hz, 1H), 8.22 (dd, J=8.0, 1.2 Hz, 1H), 7.66 (ddd, J=8.0, 4.8, 0.8 Hz, 1H) , 7.46 (d, J=3.2 Hz, 1H), 7.15 (dd, J=9.2, 2.8 Hz, 1H), 7.07 (d, J=8.8 Hz, 1H), 3.92(s, 3H):

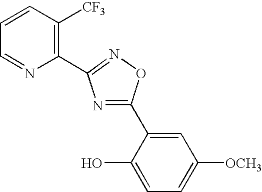

Compound 211: 5-Methoxy-2-[3-(3-trifluoromethyl-pyridin-2-yl)-[1,2,4]oxadiazol-5-yl]-phenol: 4-methoxysalicylic acid (33 mg, 0.20 mmoles) and O14 N-Hydroxy-3-trifluoromethyl-pyridine-2-carboxarnidine (40 mg, 0.20 mmoles) (100 mg, 0.49 mmoles) were subjected to protocol described above to yield 18 mg of white powder in 26% yield. [M+H]⁺ 338.1; ¹H (CDCl₃) δ 9.85 (s, 1H), 8.99(dd, J=6.0, 1.2 Hz, 1H), 8.23 (dt, J=8.0, 0.8 Hz, 1H), 7.67 (ddd, J=8.0, 4.8, 0.8), 7.46 (d, J=3.2 Hz, 1H), 7.16 (dd, J=9.2, 3.2 Hz, 1H), 7.08 (d, J=9.2 Hz, 1H), 3.90 (s, 1H):

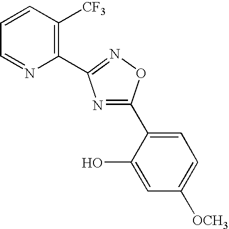

Compound 292: 4-Bromo-2-[3-(3-trifluoromethyl-pyridin-2-yl)-[1,2,4]oxadiazol-5-yl]-phenol: 5-Bromosalicylic acid (106 mg, 0.48 mmoles) and O14 N-Hydroxy-3-trifluoromethyl-pyridine-2-carboxamidine (100 mg, 0.48 mmoles) were subjected to protocol described above to yield 53 mg of white powder in 28% yield. [M+H]⁺ 386.2, 388.2; ¹H (CDCl₃) δ 10.20, (s, 1H), 8.99 (d, J=3.2Hz, 1H), 8.23 (d, J=8.0 Hz, 1H), 8.16 (d, J=2.8 Hz, 1H), 7.68 (dd, J=8.4, 4.8 Hz, 1H), 7.61 (dd, J=8.8, 2.4 Hz, 1H), 7.05 (d, J=8.8 Hz, 1H), 3.05 (s, 3H):

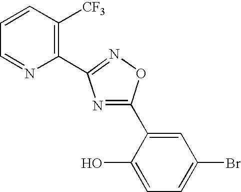

Compound 213: 4-Bromo-2-13-(5-trifluoromethyl-pyridin-2-yl)-[1 ,2,4]oxadiazol-5-yl]-phenol: N-Hydroxy-5-trifluoromethyl-pyridine-2-carboxamidine 012 (60 mg, 0.29 mrnoles) and. 5-bromosalicylic acid (1.0 equiv, 50 mg, 0.29 mmoles) were subjected to protocol described above to yield 24 mg of white powder in 21% yield. [M+H]⁺ 386.2, 388.0; ¹H (CDCl₃) δ 9.09 (s, 1H), 8.30 (d, J=7.6 Hz, 1H), 8.16 (m, 2H), 7.63 (dd, J=9.2, 2.4 Hz, 1H), 7.08 (d, J=9.2 Hz, 1H):

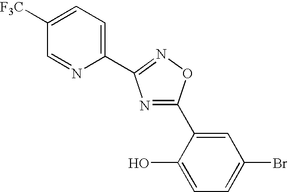

Compound 214: 2-[5-(2,5-Dimethoxy-phenyl)-[1,2,4]oxadiazol-3-yl]-5-trifluoromethyl-pyridine: N-Hydroxy-5-trifluoromethyl-pyridine-2-carboxamidine 012 (60 mg, 0.29 mmoles) and. 2,5-dimethoxybenzoic acid (1.0 equiv, 53 mg, 0.29 mmoles) were subjected to protocol described above to yield 23 mg of white powder in 23% yield. [M+H]⁺ 352.3; ¹H (CDCl₃) δ 9.07 (s, 1H), 8.37 (d, J=8.4 Hz, 1H), 8.12 (dd, J=8.4, 2.4 Hz, 1H), 7.72 (d, J=3.2 Hz, 1H), 7.15 (dd, J=9.2, 3.2 Hz, I1H), 7.04 (d, J=9.2 Hz, 1H), 3.98 (s, 3H), 3.87 (s, 3H):

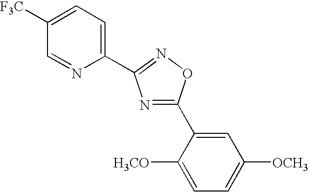

Compound 215: 2-[5-(3-Nitro-phenyl)-[11,2,4]oxadiazol-3-yl]-5-trifluoromethyl-pyridine: N-Hydroxy-5-trifluoromethyl-pyridine-2-carboxamnidine 012 (60 mg, 0.29 mmoles) and. 3-nitrobenzoic acid (1.0 equiv, 48 mg, 0.29 mmoles) were subjected to protocol described above to yield 6 mg of yellow powder in 6% yield. [M+H]⁺ 337.2; ¹H (CDCl₃) δ 9.15 (d, J=2.0 Hz, 1H), 9.11 (q, J=0.8 Hz, 1H), 8.62 (td, J=7.6, 1.2 Hz, 1H), 8.51 (ddd, J=8.4, 2.8, 1.2 Hz, 1H), 8.39 (d, J=8.4 Hz, 1H), 8.17 (dd, J=8.4, 2.0 Hz, 1H), 7.82 (t, J=8.4 Hz, 1H):

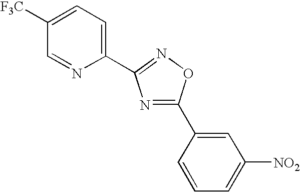

Compound 216 2-[5-(2-Chloro-4-nitro-phenyl)-[1,2,4]oxadiazol-3-yl]-5-trifluoromethyl-pyridine: N-Hydroxy-5-trifluoromethyi-pyridine-2-carboxamidine 012 (60 mg, 0.29 mmoles) and. 2-chloro-4-nitrobenzoic acid (1.0 equiv, 58 mg, 0.29 mmoles) were subjected to protocol described above to yield 44 mg of white powder in 41% yield. [M+H]⁺ 371.3, 373.3; ¹H (CDCl₃) δ 9.10 (s, 1H), 8.48 (d, J=1.6 Hz, 1H), 8.47 (dd, J=8.4, 2.4 Hz, 1H), 8.38 (dd, J=8.4, 0.8 Hz, 1H), 8.30 (dd, J=8.8, 2.4 Hz, 1H), 8.17 (dd, J=8.0, 2.0 Hz, 1H):

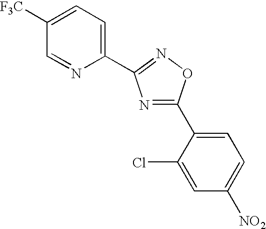

Compound 217: 2-[3-(5-Trifluoromethyl-pyridin-2-yl)-[1,2,4]oxadiazol-5-yl]-benzene-1,4-diol: N-Hydroxy-5-trifluoromethyl-pyridine-2-carboxamidine O12 (60 mg, 0.29 mmoles) and. 2,5-dihydroxybenzoic acid (1.0 equiv, 44 mg, 0.29 mmoles) were subjected to protocol described above to yield 17 mg of white powder in 18% yield. [M H]⁺: 324.2; ¹H NMR (CDCl₃) δ 10.53 (s, 1H), 9.62 (s, 1H), 9.00 (d, J=0.8 Hz, 1H), 8.71 (br s, 1H), 8.23 (d, J=8.4 Hz, 1H), 8.09 (dd, J=8.4, 2.4 Hz, 1H), 7.40 (d, J=3.2 Hz, 1H), 7.04 (dd, J=8.8, 3.2 Hz, 1H), 6.93 (d, J=9.2 Hz, 1H):

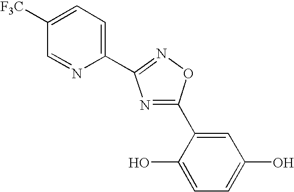

Compound 218: 4-Chloro-2-[3-(5-trifluoromethyl-pyridin-2-yl)-[1,2,4]oxadiazol-5-yl]-phenol: N-Hydroxy-5-trifluoromethyl-pyridine-2-carboxamidine O12 (60 mg, 0.29 mmoles) and. 5-chlorosalicylic acid (1.0 equiv, 50 mg, 0.29 mmoles) were subjected to protocol described above to yield 28 mg of white powder in 28% yield. [M H]⁺: 342.2, 344.2; ¹H NMR (CDCl₃) 10.20 (br s, 1H), 9.08 (s, 1H), 8.28 (d, J=8.0 Hz, 1H), 8.15 (dd, J=8.4, 2.4 Hz, 1H), 8.00 (d, J=2.8 Hz, 1H), 7.49 (dd, J=8.8, 2.4 Hz, 1H), 7.12 (d, J=9.2 Hz, 1H):

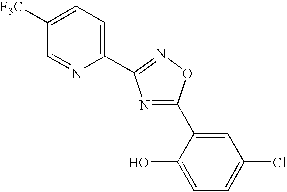

Compound 219: 4-Fluoro-2-[3-(5-trifluoromethyl-pyridin-2-yl)-[1,2,4]oxadiazol-5-yl]-phenol: N-Hydroxy-5-trifluoromethyl-pyridine-2-carboxamidine O12 (60 mg, 0.29 mmoles) and. 5-fluorosalicylic acid (1.0 equiv, 50 mg, 0.29 mmoles) were subjected to protocol described above to yield 12 mg of white powder in 13% yield. [M H]⁺: 326.2; ¹H NMR (CDCl₃) 10.06 (s, 1H), 9.09 (p, J=0.8Hz, 1H), 8.30 (dd, J=8.8, 0.8 Hz, 1H), 8.16 (dd, J=7.6, 2.0 Hz, 1H), 7.73 (dd, J=8.4, 2.0 Hz, 1H), 7.29 (ddd, J=8.8, 7.6, 2.8 Hz, 1H), 7.15 (dd, J=9.2, 4.4 Hz, 1H):

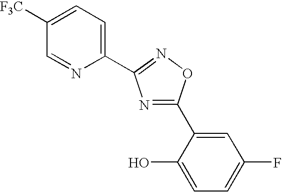

Compound 221: 1-[3-(5-Trifluoromethyl-pyridin-2-yl)-[1,2,4]oxadiazol-5-yl]-naphthalen-2-ol: N-Hydroxy-5-trifluoromethyl-pyridine-2-carboxamidine O12 (60 mg, 0.29 mmoles) and. 2-hydroxy-1-naphthoic acid (1.0 equiv, 55 mg, 0.29 mmoles) were subjected to protocol described above to yield 5 mg of a yellow wax in 5% yield. [M H]⁺: 358.2; ¹H NMR (CDCl₃) 9.11 (s, 1H), 8.94 (d, J=8.8 Hz, 1H), 8.33 (d, J=8.4 Hz, 1H), 8.17 (dd, J=8.0, 1.2 Hz, 1H), 8.00 (d, J=8.8 Hz, 1H), 7.85 (d, J=8.0 Hz, 1H), 7.73 (td, J=8.4, 1.6 Hz, 1H), 7.48 (td, J=8.0, 1.2 Hz, 1H), 7.34 (d, J=9.2 Hz, 1H):

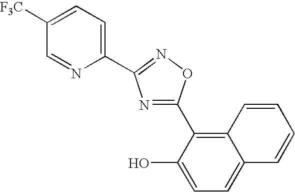

Compound 222: 5-Methoxy-2-[3-(5-trifluoromethyl-pyridin-2-yl)-[1,2,4]oxadiazol-5-yl]-phenol: N-Hydroxy-5-trifluoromethyl-pyridine-2-carboxamidine O12 (60 mg, 0.29 mmoles) and. 4-methoxysalicylic acid (1.0 equiv, 48 mg, 0.29 mmoles) were subjected to protocol described above to yield 4 mg of white powder in 4% yield. [M H]⁺: 338.3:

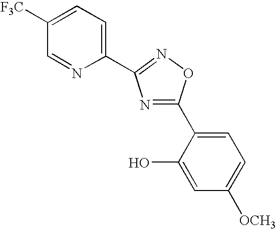

Compound 223: 2-[3-(5-Trifluoromethyl-pyridin-2-yl)-11,2,4]oxadiazol-5-yl]-phenol: N-Hydroxy-5-trifluoromethyl-pyridine-2-carboxarnidine O12 (60 mg, 0.29 mmoles) and. salicylic acid (1.0 equiv, 40 mg, 0.29 mmoles) were subjected to protocol described above to yield 11 mg of white powder in 12% yield. [M H]⁺: 308.2:

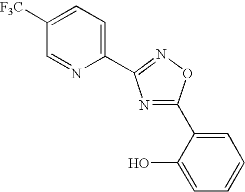

Compound 224: 4-Methoxy-2-[3-(5-trifluoromethyl-pyridin-2-yl)-[1,2,4]oxadiazol-5-yl]-phenol: N-Hydroxy-5-trifluoromethyl-pyridine-2-carboxamidine O12 (60 mg, 0.29 mmoles) and. 5-methoxysalicylic acid (1.0 equiv, 49 mg, 0.29 mmoles) were subjected to protocol described above to yield 6 mg of white powder in 6% yield. [M H]⁺: 338.3; ¹H NMR (CDCl₃) 9.85 (br s, 1H), 9.087 (s, 1H), 8.30 (d, J=8.4 Hz, 1H), 8.16 (dd, J=8.0, 1.6 Hz, 1H), 7.46 (d, J=3.2 Hz, 1H), 7.17 (m, 1H), 7.10 (d, J=9.2 Hz, 1H), 3.87 (s, 3H):

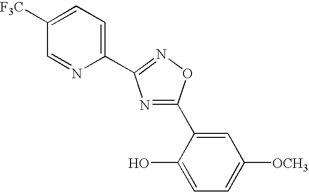

Compound 225: 4-Methyl-2-[3-(5-trifluoromethyl-pyridin-2-yl)-[1,2,4]oxadiazol-5-yl]-phenol: N-Hydroxy-5-trifluoromethyl-pyridine-2-carboxamidine O12 (60 mg, 0.29mmoles) and. 5-methylsalicylic acid (1.0 equiv, 44 mg, 0.29 mmoles) were subjected to protocol described above to yield 7 mg of white powder in 8% yield. [M H]⁺: 322.2; ¹H NMR (CDCl₃) 10.02 (s, 1H), 9.07 (s, 1H), 8.28 (d, J=8.4 Hz, 1H), 8.14 (dd, J=7.6, 2.0 Hz, 1H), 7.82 (s, 1H), 7.35 (dd, J=8.8, 2.0 Hz, 1H), 7.05 (d, J=8.8 Hz, 1H), 2.40 (s, 3H).

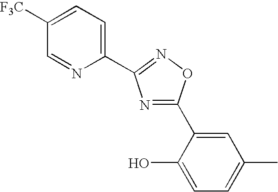

Compound 226: 2-[5-(2-Chloro-5-nitro-phenyl)-[1,2,4]oxadiazol-3-yl]-5-trifluoromethyl-pyridine: N-Hydroxy-5-trifluoromethyl-pyridine-2-carboxamidine O12 (60 mg, 0.29 mmoles) and 2-chloro-5-nitrobenzoic acid (1.0 equiv, 58 mg, 0.29 mmoles) were subjected to protocol described above to yield 27 mg of yellow powder in 25% yield. [M H]⁺: 371.2; ¹H NMR (CDCl₃) 10.02 (br s, 1H), 9.08 (s, 1H), 8.29 (d, J=8.4 Hz, 1H), 8.16 (dd, J=8.0, 2.4 Hz, 1H), 7.83 (q, J=0.8 Hz, 1H), 7.36 (dd, J=8.4, 2.4 Hz, 1H), 7.07 (d, J=8.4 Hz, 1H):

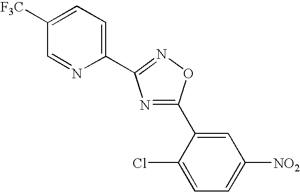

Compound 227: 2-(5-Naphthalen-1-yl-[1,2,4]oxadiazol-3-yl)-5-trifluoromethyl-pyridine: N-Hydroxy-5-trifluoromethyl-pyridine-2-carboxamidine O12 (60 mg, 0.29 mmoles) and. 1-naphthoic acid (1.0 equiv, 50 mg, 0.29 mmoles) were subjected to protocol described above to yield 5 mg of white powder in 5% yield. [M H]⁺: 342.3; ¹H NMR (CDCl₃) 9.20 (dd, J=8.4, 0.8 Hz, 1H), 9.12(s, 1H), 8.51 (dd, J=7.6, 1.6 Hz, 1H), 8.45 (d, J=8.4 Hz, 1H), 8.18 (dd, J=8.4, 2.4 Hz, 1H), 8.14 (d, J=8.4 Hz, 1H), 7.98 (d, J=8.4 Hz, 1H), 7.74 (td, J=8.0, 1.2 Hz, 1H), 7.65 (m, 2H):

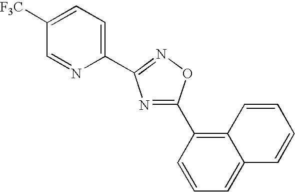

Compound 265: 2,3-Dimethoxy-6-[3-(5-trifluoromethyl-pyridin-2-yl)-[1,2,4]oxadiazol-5-yl]-phenol: N-Hydroxy-5-trifluoromethyl-pyridine-2-carboxamidine O12 (60 mg, 0.29 mmoles) and. 5-methoxysalicylic acid (1.0 equiv, 49 mg, 0.29 mmoles) were subjected to protocol described above to yield 6 mg of white powder in 6% yield. [M H]⁺: 368.3 ; ¹H NMR (CDCl₃) 10.23 (br s, 1H), 9.07 (s, 1H), 8.27 (d, J=8.4 Hz, 1H), 8.15 (dd, J=8.0, 2.0, 1H), 7.78 (d, J=9.2 Hz, 1H), 6.68 (d, J=8.8 Hz, 1H), 3.98 (s, 3H), 3.96 (s, 3H):

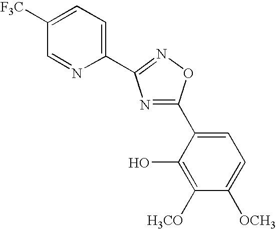

Compound 269: 2-Ethoxy-6-[3-(5-trifluoromethyl-pyridin-2-yl)-[1,2,4]oxadiazol-5-yl]-phenol: N-Hydroxy-5-trifluoromethyl-pyridine-2-carboxamidine 012 (102 mg, 0.50 mmoles) and. 4-ethoxysalicylic acid (1.0 equiv, 91 mg, 0.50 mmoles) were subjected to protocol described in example 1 to yield 1 mg of white powder in 0.6% yield. [M H]⁺: 352.2; ¹H NMR (CDCl₃) δ 810.21 (s, 1H), 9.075 (s, 1H), 8.26 (d, J=8.0 Hz, 1H), 8.16 (dd, J=8.0, 2.4 Hz, 1H), 7.65 (dd, J=7.6, 1.2 Hz, 1H), 7.12 (d, J=8.0 Hz, 1H), 6.995 (t, J=8.0 Hz, 1H), 4.19 (q, J=6.8 Hz, 2H), 1.53 (t, J=6.8 Hz, 1H):

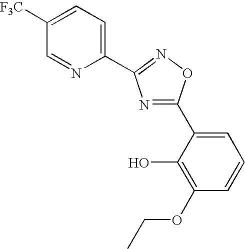

### EXAMPLE 10

Compound 287: 4-Trifluoromethoxy-2-[3-(5-trifluoromethyl-pyridin-2-yl)-[1,2,4]oxadiazol-5-yl]-phenol (287)

Step 1: Preparation of O16 5-(trifluoromethoxy) salicylic acid

5-(trifluoromethoxy)salicylaldehyde (400 mg, 1.94 mmoles) was dissolved in THF (4 ml) and 2-methyl-2-propanol (20 ml). 2-methyl-2-butene (10 equiv., 9 ml of 2.0 N in THF, 17.5 mmoles) was then added. This was quickly followed by the addition of a separate solution of sodium chlorite (3.0 equiv, 526 mg, 5.82 mmoles) and sodium hydrogensulfate monohydrate(3.0 equiv., 803 mg, 5.82 mmoles) dissolved in water (4 ml). The reaction stirred at ambient temperature for 2 hours. The solvents were removed in vacuo, and the residue was partitioned between Ethyl Acetate and 0.1 N aqueous HCl. The organic layer was separated and dried over magnesium sulfate, then purified by chromatographic separation to produce 389 mg of brown waxy solid in 90% yield. [M H]⁺: 221.3; ¹H (d₆-DMSO) δ 7.64 (d, J=2.4 Hz, 1H), 7.51 (dd, J=8.8, 2.2 Hz, 1H), 7.05 (d, J=8.8 Hz, 1H).

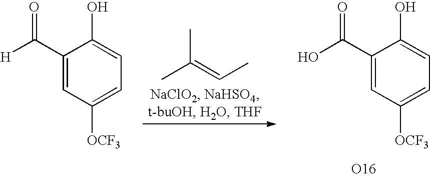

Step 2

Compound 287: 4-Trifluoromethoxy-2-[3-(5-trifluoromethyl-pyridin-2-yl)-[1,2,4]oxadiazol-5-yl]-phenol.

N-Hydroxy-5-trifluoromethyl-pyridine-2-carboxamidine 012 (92 mg, 0.45 mmoles) and. 5-trifluoromethoxysalicylic acid (1.0 equiv, 100 mg, 0.45 mmoles) were subjected to protocol described in example 8 to yield 45 mg of white powder in 25% yield. [M H]⁺: 392.3; ¹H (CDCl₃) δ 10.30 (s, 1H), 9.10 (s, 1H), 8.31 (d, J=8.0 Hz, 1H), 8.17 (d, J=8.0 Hz, 1H), 7.91 (d, J=2.4 Hz, 1H), 7.44 (dd, J=9.2, 2.8 Hz, 1H), 7.21 (d, J=9.2 Hz, 1H):

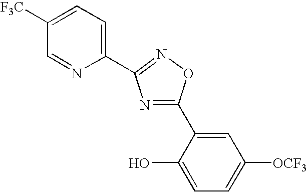

### EXAMPLE 11

Compound 133: 2-(3-Pyridin-2-yl-[1,2,4]oxadiazol-5-yl)-phenol

Step 1: Preparation of O17 N-Hydroxy-pyridine-2-carboxamidine

2-cyanopyridine using was subjected to conditions outlined in Example 8 to yield 12.4 g of desired product in 94% yield. ¹H (CDCl₃) δ 8.55 (ddd, J=4.8, 1.6, 1.2 Hz, 1H), 7.93 (dt, J=8.0, 1.2 Hz, 1H), 7.70 (td, J=7.2, 1.6), 5.75 (b, 2H):

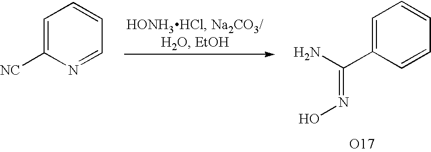

Step 2

Compound 133: 2-(3-Pyridin-2-yl-[1,2,4]oxadiazol-5-yl)-phenol

Synthesis was carried out with salicylic acid and O17 N-Hydroxy-pyridine-2-carboxamidine using protocol outlined in example 8 to produce 36 mg of white solid in 21% yield. [M+H]⁺ 240.1; ¹H (CDCl₃) δ 8.79 (s, 1H), 8.12 (d, J=7.6Hz, 1H), 7.99 (d, J=7.6 Hz, 1H)., 7.87(dd, J=7.2,6.4 Hz, 1H), 7.47 (m, 2H), 7.11 (d, , J=8.4, 1H), 7.01 (m, 2H); ¹³C (CDCl₃) δ 166.92, 158.21, 150.59, 145.68, 137.34, 135.55, 128.05, 126.06, 123.60, 120.32, 118.05, 108.16:

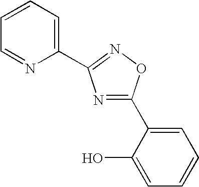

Compound 134: 1-(3-Pyridin-2-yl-[1,2,4]oxadiazol-5-yl)-naphthalen-2-ol:

O17 N-Hydroxy-pyridine-2-carboxamidine and 2-hydroxy-1-naphthoic acid were subjected to conditions outlined in example 8 to produce 95 mg of white powder in 22% yield. [M+H]⁺ 290.1; ¹H (CDCl₃) δ 8.79 (t, J=8.8, 1H), 8.71 (d, J=4.4, 1H), 8.03 (t, J=7.6, 1H), 7.78 (m, 2H), 7.67, (t, J=8.4, 1H), 7.54, (dd, J=7.6, 7.2, 1H), 7.32, (m, 2H), 7.16 (t, 8.4, 1H); ¹³C (CDCl₃) δ 150.24, 137.91, 136.94, 135.01, 130.78, 129.55, 129.39, 128.64, 126.31, 124.57, 124.54, 123.83, 122,30, 119.46:

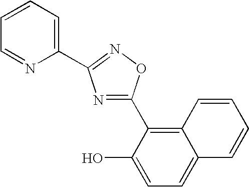

Compound 139: 2-[5-(3-Methoxy-phenyl)-[1,2,4]oxadiazol-3-yl]-pyridine:

O17 N-Hydroxy-pyridine-2-carboxamidine and 3-methoxybenzoic acid were subjected to conditions outlined in example 8 to produce 44 mg of white powder in 35% yield. [M+H]⁺: 254.3:

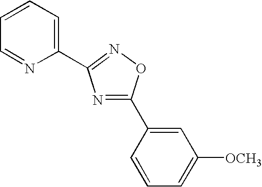

Compound 149: 2-(5-Naphthalen-1-yl-[1,2,4]oxadiazol-3-yl)-pyridine:

O17 N-Hydroxy-pyridine-2-carboxamidine and 3-methoxybenzoic acid were subjected to conditions outlined in example XX to produce 20 mg of white powder in 15% yield. [M+H]⁺: 274.2:

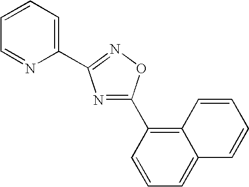

Compound 141: 2-[5-(2-Chloro-4-nitro-phenyl)-[1,2,4]oxadiazol-3-yl]-pyridine:

O17 N-Hydroxy-pyridine-2-carboxamidine and 2-chloro-4-nitrobenzoic acid were subjected to conditions outlined in example 8 to produce 42 mg of white powder in 27% yield. [M+H]⁺: 303.1:

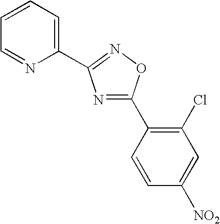

Compound 142: 2-[5-(2,5-Dimethoxy-phenyl)-[1,2,4]oxadiazol-3-yl]-pyridine:

O17 N-Hydroxy-pyridine-2-carboxamidine 2,5-dimethoxybenzoic acid were subjected to conditions outlined in example 8 to produce 48 mg of white powder in 34% yield. [M+H]⁺: 284.2

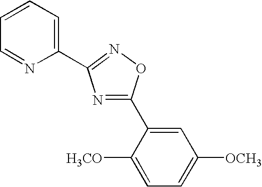

Compound 143: 2-[5-(2-Methoxy-naphthalen-1-yl)-[1,2,4]oxadiazol-3-yl]-pyridine:

O17 N-Hydroxy-pyridine-2-carboxamidine and 2-methoxy-1-naphthoic acid were subjected to conditions outlined in example 8 to produce 13 mg of brown waxy solid in 9% yield. [M+H]⁺: 304.2:

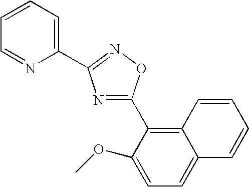

Compound 144: 4-Bromo-2-(3-pyridin-2-yl-[1,2,4]oxadiazol-5-yl)-phenol:

O17 N-Hydroxy-pyridine-2-carboxamidine and 5-bromosalicylic acid were subjected to conditions outlined in example 8 to produce 87 mg of white powder in 37% yield. [M+H]⁺: 318.2, 320.0; ¹H (CDCl₃) δ 10.37 (s, 1H), 8.84 (ddd, J=4.8, 1.6, 0.8 Hz, 1H), 8.15 (m, 2H), 7.91 (td, J=8.0, 2.0 Hz, 1H), 7.61 (dd, J=8.8, 2.8 Hz, 1H), 7.50 (ddd, J=7.6, 4.8, 1.2 Hz, 1H), 7.06 (d, J=8.8 Hz, 1H):

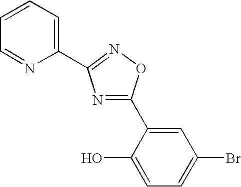

Compound 145: 4-Chloro-2-(3-pyridin-2-yl-[1,2,4]oxadiazol-5-yl)-phenol:

O17 N-Hydroxy-pyridine-2-carboxamidine and 5-chlorosalicylic acid were subjected to conditions outlined in example 8 to produce 95 mg of white powder in 48% yield. [M+H]⁺: 274.2:

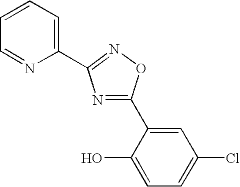

Compound 146: 4-Nitro-2-(3-pyridin-2-yl-[1,2,4]oxadiazol-5-yl)-phenol:

N-Hydroxy-pyridine-2-carboxamidine and 5-nitrosalicylic acid were subjected to conditions outlined in example 8 to produce 61 mg of white powder in 29% yield. [M+H]⁺: 285.2:

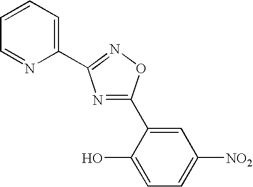

Compound 147: 4-Methoxy-2-(3-pyridin-2-yl-[1,2,4]oxadiazol-5-yl)-phenol:

O17 N-Hydroxy-pyridine-2-carboxamidine and 5-methoxysalicylic acid were subjected to conditions outlined in example 8 to produce 38 mg of white powder in 19% yield. [M+H]⁺: 270.1; ¹H (CDCl₃) δ 8.92 (d, J=4.8 Hz, 1H) 8.23 (dt, J=4.4, 0.8 Hz, 1H), 7.97 (td, J=7.6, 1.2 Hz, 1H), 7.57 (ddd, J=8.8, 4.8, 0.8 Hz, 1H), 7.46 (d, J=2.8 hz, 1H), 7.16 (dd, J=9.2, 3.6 Hz, 1H), 7.11 (d, J=8.8 Hz, 1H), 3.89 (s, 3H):

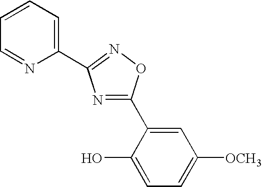

Compound 148: 2-[5-(3-Nitro-phenyl)-[1,2,4]oxadiazol-3-yl]-pyridine:

O17 N-Hydroxy-pyridine-2-carboxamidine and 3-nitrobenzoic acid were subjected to conditions outlined in example 8 to produce 31 mg of white powder in 16% yield. [M+H]⁺: 269.3:

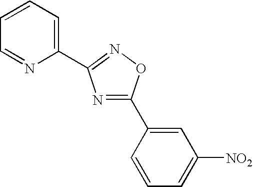

Compound 149: 2-[5-(2-Fluoro-5-nitro-phenyl)-1l,2,4]oxadiazol-3-yl]-pyridine:

O17 N-Hydroxy-pyridine-2-carboxamidine and 2-fluoro-5-nitrobenzoic acid were subjected to conditions outlined in example 8 to produce 61 mg of white powder in 29% yield. [M+H]⁺: 287.1:

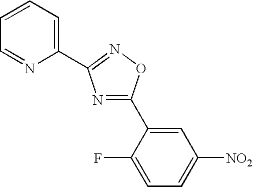

Compound 150: 2-(3-Pyridin-2-yl-[1,2,4]oxadiazol-5-yl)-benzene-1,4-diol:

O17 N-Hydroxy-pyridine-2-carboxamidine and 2,5,dihydroxybenzoic acid were subjected to conditions outlined in example 8 to produce 15 mg of white powder in 6% yield. [M+H]⁺: 256.1:

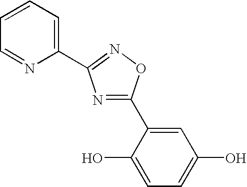

Compound 151: 2-Methoxy-6-(3-pyridin-2-yl-[1,2,4]oxadiazol-5-yl)-phenol:

O17 N-Hydroxy-pyridine-2-carboxamidine and 3-methoxysalicylic acid were subjected to conditions outlined in example 8 to produce 36 mg of white powder in 21% yield. [M+H]⁺: 270.2:

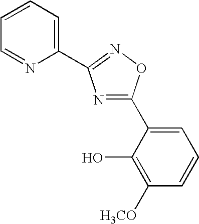

Compound 152: 5-Methoxy-2-(3-pyridin-2-yl-[1,2,4]oxadiazol-5-yl)-phenol:

O17 N-Hydroxy-pyridine-2-carboxamidine and 4-methoxysalicylic acid were subjected to conditions outlined in example 8 to produce 23 mg of white powder in 12% yield. [M+H]⁺: 270.2:

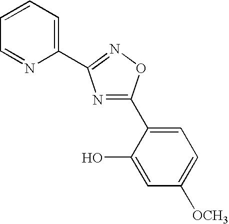

Compound 163: 5-Dimethylamino-2-(3-pyridin-2-yl-[1,2,4]oxadiazol-5-yl)-phenol:

O17 N-Hydroxy-pyridine-2-carboxamidine and 4-dimethylaminosalicylic acid were subjected to conditions outlined in example 8 to produce 51 mg of white powder in 25% yield. [M+H]⁺: 283.1; ¹H (CDCl₃) δ 8.82 (dd, J=4.0, 2.0 Hz, 1H), 8.13 (dt, J=8.0, 0.8 Hz, 1H), 7.88 (td, J=8.0, 1.6 Hz, 1H), 7.82 (d, J=8.8Hz, 1H), 7.46 (ddd, J=8.0, 4.8, 1.2 Hz, 1H), 6.39 (dd, J=9.2, 2.4 Hz, 1H), 6.31 (d, J=2.4 Hz, 1H), 3.09 (s, 6H):

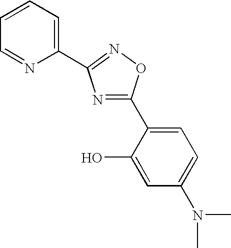

Compound 164: 4-Fluoro-2-(3-pyridin-2-yl-[1,2,4]oxadiazol-5-yl)-phenol:

O17 N-Hydroxy-pyridine-2-carboxamidine and 5-fluorosalicylic acid were subjected to conditions outlined in example 8 to produce 28 mg of white powder in 15% yield. [M+H]⁺: 258.0; ¹H (CDCl₃) δ 8.85 (dt, J=4.8, 0.8 Hz, 1H), 8.16 (dd, J=8.0, 1.2 Hz, 1H), 7.91 (td, J=8.0, 2.0 Hz, 1H), 7.70 (dd, J=8.4, 3.2 Hz, 1H), 7.50 (ddd, J=8.0, 4.8, 1.2 Hz, 1H), 7.26 (m, 1H), 7.12 (dd, J=9.2, 4.8 Hz, 1H):

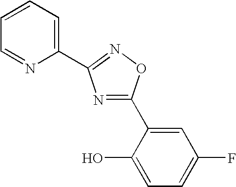

Compound 172: 4-Methyl-2-(3-pyridin-2-yl-[1,2,4]oxadiazol-5-yl)-phenol:

O17 N-Hydroxy-pyridine-2-carboxamidine and 5-methylsalicylic acid were subjected to conditions outlined in example 8 to produce 51 mg of white powder in 28% yield. [M+H]⁺: 254.2:

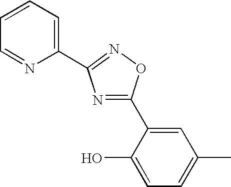

Compound 173: 2-[5-(2-Chloro-5-nitro-phenyl)-[1,2,4]oxadiazol-3-yl]-pyridine:

O17 N-Hydroxy-pyridine-2-carboxamidine and 2-chloro-5-nitrobenzoic acid were subjected to conditions outlined in example 8 to produce 32 mg of white powder in 15% yield. [M+H]⁺: 303.1:

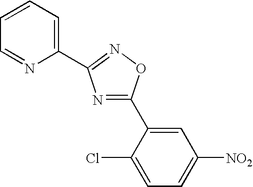

Compound 281: 3,6-Dichloro-2-(3-pyridin-2-yl-[1,2,4]oxadiazol-5-yl)-phenol:

Step 1: Preparation of O18 3,5-dichlorosalicylic acid: 3,5-dichlorosalicylaldehyde (200 mg, 1.04 mmoles) was subjected to the protocol described in example 10 to yield 210 mg of a white solid in 98% yield. R_{f}: 0.33 (9:1 CH₃Cl/THF); ¹H (d₆-DMSO) δ 11.0 (s, 1H), 7.81 (d, J=2.8, 1H), 7.70 (d, J=2.4, 1H); ¹³C (DMSO) δ 170.91, 156.45, 135.00, 128.78, 122.86, 122.67, 116.43

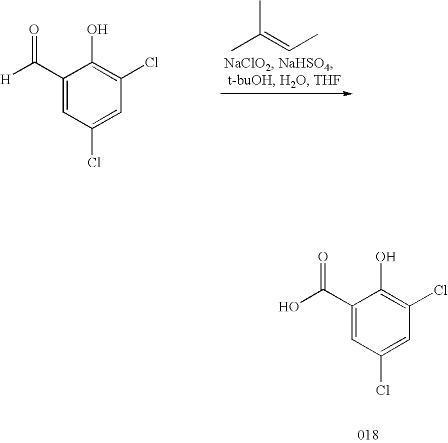

Step 2

Compound 281: 3,6-Dichloro-2-(3-pyridin-2-yl-[1,2,4]oxadiazol-5-yl)-phenol O18 3,5-dichlorosalicylic acid and O17 N-Hydroxy-pyridine-2-carboxamidine were subjected to the protocol outlined in Example 8 to yield 6.4 mg of white powder in 5% yield.; [M+H]⁺ 308.2, 310.2; ¹H (CDCl3) δ 8.85 (dt, J=4.8, 1.2 Hz, 1H), 8.14 (dd, J=7.6, 1.2 Hz, 1H), 7.94 (m, 2H), 7.63 (dd, J=1.6,1.2 Hz, 1H), 7.52 (ddd, J=6.8, 4.8, 1.2 Hz, 1H):

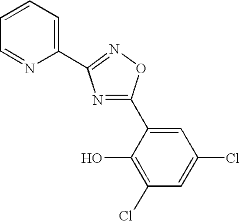

### EXAMPLE 12

Compound 137: 1-[3-(3-Nitro-pyridin-2-yl)-[1,2,4]oxadiazol-5-yl]-naphthalen-2-ol

Step 1: Preparation of O19 3-Nitro-pyridine-2-carbonitrile. 2-Bromo-3-nitropyridine (10 g, 49 mmoles) was dissolved in dry DMF (150 mL) under Argon. Tetrakis(triphenylphosphine) palladium(O) (0.06 equiv, 3.4 g, 2.94 mmoles) and zinc cyanide (0.6 equiv, 3.45 g, 29.4 mmoles) was then added. The reaction stirred at reflux for 15 h. The mixture was then concentrated in vacuo, and the resulting residue was partitioned between ethyl acetate and water, then brine. Dried over magnesium sulfate. The organic layer was then purified by chromatographic separation to yield 5.9 g of the desired product as a yellow solid in 81% yield; [M]⁻: 148.1; ¹H (CDCl3) δ 9.01 (dd, J=4.8, 1.6, Hz, 1H), 8.62 (dd, J=8.4, 1.2), 7.81 (dd, J=8.4, 4.8)

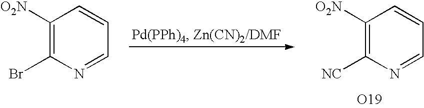

Step 2: Preparation of O20 N-Hydroxy-3-nitro-pyridine-2-carboxamidine. Synthesis was carried out using O19 3-Nitro-pyridine-2-carbonitrile and protocol outlined in Example 1 to produce 6.Og of the desired product in 88% yield. [M H]⁺: 183.1; ¹H (CDCl₃) δ 10.14 (s, 1H), 8.79 (dd, J=4.8, 1.6 Hz, 1H), 8.24 (dd, J=8.0, 1.2 Hz, 1H), 7.65 (dd, J=8.0, 4.8), 5.96 (s, 2H):

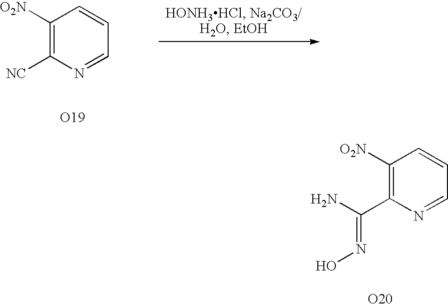

Step 3

Compound 137: 1-13-(3-Nitro-pyridin-2-yl)-[1,2,4]oxadiazol-5-yl]-naphthalen-2-ol.

O20 N-Hydroxy-3-nitro-pyridine-2-carboxamidine and 2-hydroxynaphthoic acid were subjected to conditions outlined in Example 8 to produce 35 mg of desired product [137] in 32% yield. ¹H (CDCl₃) δ 11.75 (s, 1H), 9.03 (dd, , J=4.8, 1.2 Hz, 1H), 8.92 (d, 8.8 Hz, 1H), 8.39 (dd, J=8.4, 1.6 Hz, 1H), 7.98 (d, J=9.2 Hz, 1H), 7.84 (d, J=8.0 Hz, 1H), 7.73 (m, 2H), 7.48 (t, J=7.2 Hz, 1H), 7.35 (d, J=9.2 Hz, 1H):

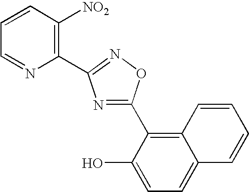

4-Chloro-2-[3-(3-nitro-pyridin-2-yl)-[1,2,4]oxadiazol-5-yl]-phenol (154):

O20 N-Hydroxy-3-nitro-pyridine-2-carboxamidine and 5-chlorosalicylic acid were subjected to conditions outlined in Example 8 to produce 60 mg of desired product in 31% yield. ¹H (d₆-DMSO) δ 9.05 (dd, J=4.4, 1.2 Hz, 1H), 8.64 (dd, J=8.0, 1.2 Hz, !H), 7.96 (dd, J=8.4, 4.8 Hz, 1H), 7.88 (d, J=2.8 Hz, 1H), 7.57 (dd, J=8.8, 2.8 Hz, 1H), 7.13 (d, J=9.2 Hz, 1H):

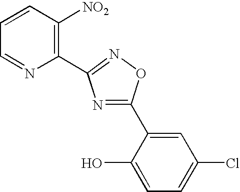

4-Bromo-2-[3-(3-nitro-pyridin-2-yl)-[1,2,4]oxadiazol-5-yl]-phenol (155):

O20 N-Hydroxy-3-nitro-pyridine-2-carboxamidine and 5-bromosalicylic acid were subjected to conditions outlined in Example 8 to produce 52 mg of desired product in 24% yield. ¹H (CDCl₃) δ 9.89 (s, 1H), 9.02 (dd, J=4.8, 1.6 hz, 1H), 8.39 (dd, , J=8.8, 1.6 Hz, 1H), 8.15 (d, J=2.4 Hz, 1H) 7.75 (dd, J=8.4, 4.8 Hz, 1H), 7.62 (dd, J=8.8, 2.4 Hz, 1H), 7.04 (d, J=9.2 Hz, 1H):

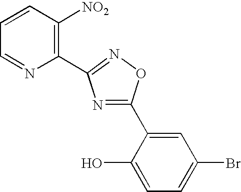

3-Nitro-2-[5-(3-nitro-phenyl)-[1,2,4]oxadiazol-3-yl]-pyridine (156):

O20 N-Hydroxy-3-nitro-pyridine-2-carboxamidine and 3-nitrobenzoic acid were subjected to conditions outlined in Example 8 to produce 16 mg of desired product in 9% yield. [M H]⁺: 313.8; ¹H (CDCl₃) δ 9.07 (t, J=1.6Hz, 1H), 9.03 (dd, J=4.8, 1.6 Hz, 1H), 8.54 (dt, J=8.0, 2.0 Hz, 1H), 8.50 (ddd, J=8.4, 2.4, 1.2 Hz, 1H), 8.37 (dd, J=8.4, 1.6 Hz, 1H), 7.80 (t, J=8.OHz, 1H), 7.74 (dd, J=8.4, 4.8 Hz, 1H):

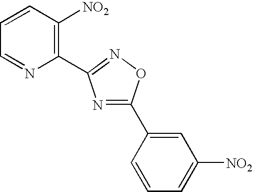

2-(5-Naphthalen-1-yl-[1,2,4]oxadiazol-3-yl)-3-nitro-pyridine (157):

O20 N-Hydroxy-3-nitro-pyridine-2-carboxamidine and l-naphthoic acid were subjected to conditions outlined in Example 8 to produce 36 mg of desired product in 19% yield. [M H]⁺: 319.2; ¹H (CDCl₃) δ 9.07 (d, J=8.4 Hz, 1H), 9.01 (dd, J=4.8, 1.6 Hz, 1H), 8.43 (dd, J=7.6, 1.2 Hz, 1H), 8.33 (dd, J=8.0, 1.2 Hz, 1H), 8.11, (d, J=8.4 Hz, 1H), 7.93 (dd, J=8.0, 0.8 Hz, 1H), 7.70 (m, 2H), 7.60 (m, 2H):

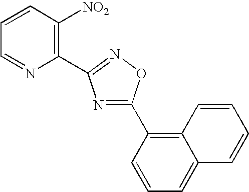

4-Nitro-2-[3-(3-nitro-pyridin-2-yl)-[1,2,4]oxadiazol-5-yl]-phenol (158):

O20 N-Hydroxy-3-nitro-pyridine-2-carboxamidine and 5-nitrosalicylic acid were subjected to conditions outlined in Example 8 to produce 25 mg of desired product in 13% yield. ¹H (CDCl₃) δ 9.05 (dd, J=4.8, 04 Hz, 1H), 8.73 (d, J=2.8Hz, 1H), 8.64 (dd, J=8.4, 1.6 Hz, 1H), 8.36 (dd, J=9.6, 2.8Hz, 1H), 7.95 (dd, J=8.0, 4.8 Hz, 1H), 7.28 (d, J=9.2 Hz, 1H):

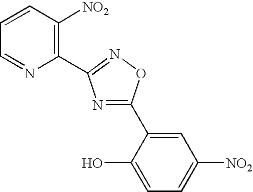

2-[5-(2,5-Dimethoxy-phenyl)-[1,2,4]oxadiazol-3-ylp9 -3-nitro-pyridine (159):

O20 N-Hydroxy-3-nitro-pyridine-2-carboxamidine and 2,5-dimethoxybenzoic acid were subjected to conditions outlined in Example 8 to produce 42 mg of desired product in 21% yield. [M H]⁺: 329.1 ¹H (CDCl₃) δ 8.99 (dd, J=4.8, 1.6 Hz, 1H), 8.30 (dd, J=8.4, 1.6Hz, 1H), 7.66 (m, 2H), 7.13 (dd, J=9.2, 2.8 Hz, 1H), 7.03 (d, J=9.2 Hz, 1H), 3.96 (s, 3H), 3.84 (s, 3H):

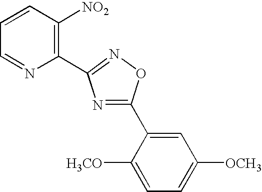

4-Methoxy-2-[3-(3-nitro-pyridin-2-yl)-[1,2,4]oxadiazol-5-yl]-phenol (160):

O20 N-Hydroxy-3-nitro-pyridine-2-carboxamidine and 5-methoxysalicylic acid were subjected to conditions outlined in Example 8 to produce 19 mg of desired product in 10% yield. [M H]⁺: 315.2; ¹H (CDCl₃) δ 9.52 (s, 1H), 9.01 (dd, J=4.8, 1.6 Hz, 1H), 8.37 (dd, J=8.8, 1.6 Hz, 1H), 7.74 (dd, J=8.4, 4.8 Hz, 1H), 7.44 (d, J=3.2 Hz, 1H), 7.15 (dd, J=9.2, 3.2 Hz, 1H), 7.06 (d, J=9.2 Hz, 1H), 3.87 (s, 3H).

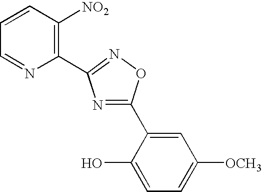

2-[3-(3-Nitro-pyridin-2-yl)-[1,2,4]oxadiazol-5-yl]-phenol (161):

N-Hydroxy-3-nitro-pyridine-2-carboxamidine and salicylic acid were subjected to conditions outlined in Example 8 to produce 46 mg of desired product in 27% yield. [M H]⁺: 285.2; ¹H (CDCl₃) δ 9.01 (dd, J=4.8, 1.6 Hz, 1H), 8.37 (dd, J=8.0, 1.2 Hz, 1H), 8.03 (dd, J=8.0, 1.6 Hz, 1H), 7.74 (dd, J=8.0, 4.8 Hz, 1H), 7.14 (d, J=8.8 Hz, 1H), 7.062 (t, J=8.0 Hz, 1H):

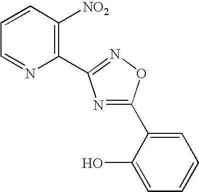

4-Fluoro-2-[3-(3-nitro-pyridin-2-yl)-[1,2,4]oxadiazol-5-yl]-phenol (162):

O20 N-Hydroxy-3-nitro-pyridine-2-carboxamidine and 5-fluorosalicylic acid were subjected to conditions outlined in Example 8 to produce 20 mg of desired product in 11% yield. ¹H (CDCl₃) δ 9.72 (s, 1H), 9.02 (dd, J=4.4, 1.6 Hz, 1H), 8.39 (dd, J=8.0, 1.2 Hz, 1H), 7.73 (m, 2H), 7.28 (m, 1H), 7.11 (dd, J=9.2, 4.4 Hz, 1H):

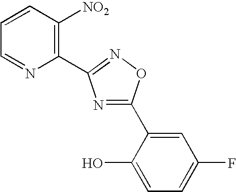

2-[5-(3-Methoxy-phenyl)-[1,2,4]oxadiazol-3-yl]-3-nitro-pyridine (165):

O20 N-Hydroxy-3-nitro-pyridine-2-carboxamidine and 3-methoxybenzoic acid were subjected to conditions outlined in example 8 to produce 29 mg of desired product in 16% yield. [M H]⁺: 299.1; ¹H (CDCl₃) δ 9.01 (dd, J=4.8, 1.2 Hz, 1H), 8.32 (dd, J=8.0, 1.2Hz, 1H), 7.81 (td, J=7.6, 1.2 Hz, 1H), 7.70 (m, 2H), 7.46 (t, J=8.0 Hz, 1H), 3.91 (s, 3H):

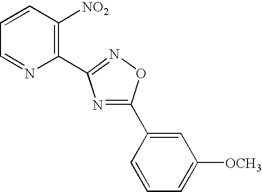

2-Methoxy-6-[3-(3-nitro-pyridin-2-yl)-[1,2,4]oxadiazol-5-yl]-phenol (166):

O20 N-Hydroxy-3-nitro-pyridine-2-carboxamidine and 3-methoxysalicylic acid were subjected to conditions outlined in example 8 to produce 13 mg of desired product in 7% yield. [M H]⁺: 315.1; ¹H (CDCl₃) δ 9.92 (s, 1H), 9.02 (dd, J=4.8, 1.2 Hz, 1H), 8.37 (dd, J=8.4, 1.6 Hz, 1H), 7.74 (dd, J=8.4, 4.4 Hz, 1H), 7.64 (dd, J=8.0, 1.6 Hz, 1H), 7.13 (dd, J=8.0, 1.6 Hz, 1H), 7.02 (t, J=8.0 Hz, 1H), 3.96 (s, 3H):

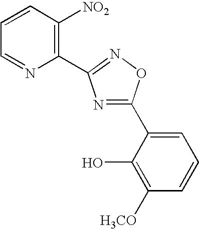

2-[5-(2-Chloro-4-nitro-phenyl)-[1,2,4]oxadiazol-3-yl]-3-nitro-pyridine (167):

O20 N-Hydroxy-3-nitro-pyridine-2-carboxamidine and 2-chloro-4-nitrobenzoic acid were subjected to conditions outlined in Example 8 to produce 29 mg of desired product in 14% yield. ¹H (CDCl₃) δ 9.03 (dd, J=4.8, 1.6 Hz, 1H), 8.47 (d, J=2.0 Hz, 1H), 8.40 (d, J=8.8 Hz, 1H), 8.37 (dd, J=8.8, 1.2 Hz, 1H), 8.29 (ddd, J=8.8, 2.4, 0.8 Hz, 1H), 7.74 (dd, J=9.2, 4.8, 0.8 Hz, 1H):

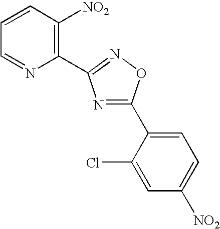

2-[5-(2-Fluoro-5-nitro-phenyl)-[1,2,4]oxadiazol-3-yl]-3-nitro-pyridine (168):

O20 N-Hydroxy-3-nitro-pyridine-2-carboxamidine and 5-fluorosalicylic acid were subjected to conditions outlined in Example 8 to produce 21 mg of desired product in 11% yield. [M H]⁺: 332.1; ¹H (d₆-DMSO) δ 9.07 (dd, J=4.8, 1.2 Hz, 1H), 8.50 (dd, J=6.0, 3.2 Hz, 1H), 8.67 (dd, J=8.4, 1.6 Hz, 1H), 8.62 (m, 1H), 7.99 (dd, J=8.0, 4.8 Hz, 1H), 7.85 (t, J=8.8 Hz, 1H):

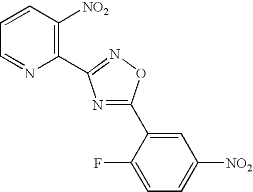

2-15-(2-Methoxy-naphthalen-1-yl)-[1,2,4]oxadiazol-3-yl]-3-nitro-pyridine (169):

O20 N-Hydroxy-3-nitro-pyridine-2-carboxamidine and 2-methoxy-1-naphthoic acid were subjected to conditions outlined in Example 8 to produce 14 mg of desired product in 7% yield. ¹H (CDCl₃) δ 8.11 (m, 2H), 7.89 (d, J=8.0 Hz, 1H), 7.64 (m, 3H), 7.48 (m, 2 H), 7.42 (d, J=9.2 hz, 1H), 4.15 (s, 3H):

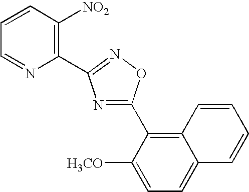

5-Methoxy-2-[3-(3-nitro-pyridin-2-yl)-[1,2,4]oxadiazol-5-yl]-phenol (170):

O20 N-Hydroxy-3-nitro-pyridine-2-carboxamidine and 4-methoxysalicylic acid were subjected to conditions outlined in Example 8 to produce 13 mg of desired product in 7% yield. [M H]⁺: 315.2; ¹H (CDCl₃) δ 10.01 (br s, 1H), 9.00 (dd, J=8.4, 1.6 Hz, 1H), 7.91 (d, J=8.4, 1H), 7.72 (dd, J=8.4, 4.8 Hz, 1H), 6.63 (dd, J=8.8, 2.4 Hz, 1H), 6.60, (d, J=2.4 Hz, 1H), 3.87 (s, 3H):

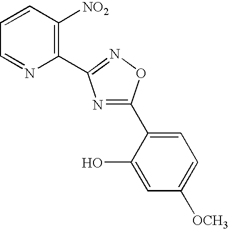

2-[3-(3-Nitro-pyridin-2-yl)-[1,2,4]oxadiazol-5-yl]-benzene-1,4-diol (171):

O20 N-Hydroxy-3-nitro-pyridine-2-carboxamidine and 2,5-dihydroxybenzoic acid were subjected to conditions outlined in Example 8 to produce 16 mg of desired product in 9% yield. [M H]⁺: 301.1; ¹H (CDCl₃/d₆-DMSO) δ 9.16 (br s, 2H), 8.82 (dd, J=4.8, 1.2 Hz, 1H), 8.22 (dd, J=8.0, 1.2 Hz, 1H), 7.59 (dd, J=8.0, 4.8 Hz, 1H), 7.25 (d, J=2.8 Hz, 1H), 6.90 (dd, J=9.2, 3.2 Hz, 1H), 6.76 (d, J=9.2 Hz, 1H):

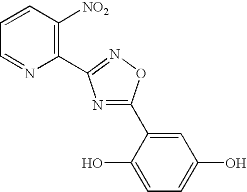

4-Methyl-2-[3-(3-nitro-pyridin-2-yl)-[1,2,4]oxadiazol-5-yl]-phenol (174):

O20 N-Hydroxy-3-nitro-pyridine-2-carboxamidine and 5-methylsalicylic acid were subjected to conditions outlined in Example 8 to produce 51 mg of desired product in 28% yield. [M H]⁺: 299.1:

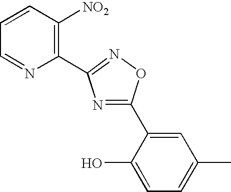

### EXAMPLE 13

4-Methoxy-2-13-(3-methyl-pyridin-2-yl)-[1,2,41oxadiazol-5-yl]-phenol (185)

Step 1: Preparation of O21 N-Hydroxy-3-methyl-pyridine-2-carboxamidine. The synthesis was carried out with 3-methylpicolonitrile as outlined by the protocol in Example 8 to yield 6.3 g of white powder in 72% yield. ¹H NMR (CDCl3) δ 8.43 (d, J=3.6 Hz, 1H), 7.55 (ddd, J=8.0, 1.2, 0.4 Hz, 1H), 7.21 (dd, J=7.6, 4.4 Hz, 1H), 5.65 (b, 3H).

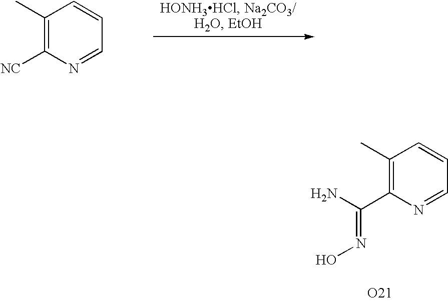

Step 2: Preparation of 4-Methoxy-2-[3-(3-methyl-pyridin-2-yl)-[1,2,4]oxadiazol-5-yl]-phenol (185). O21 N-Hydroxy-3-methyl-pyridine-2-carboxamidine and 5-methoxysalicylic acid were subjected to conditions outlined in Example 8 to produce 23 mg of desired product in 12% yield. [M H]⁺: 284.0 ¹H(CDCl₃) δ 10.07 (s, 1H), 8.67 (dd, J=4.8, 2.0 Hz, 1H), 7.70 (d, J=7.6 Hz, 1H), 7.47 (d, J=3.2 Hz, 1H), 7.38 (dd, J=8.0, 4.8 Hz, 1H), 7.15 (dd, J=8.8, 2.8 Hz, 1H), 7.08 (d, J=9.2 H, 1H), 3.85 (s, 3H), 2.65 (s, 3H):

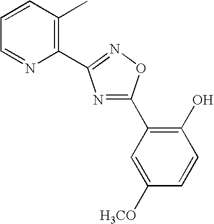

2-Methoxy-6-[3-(3-methyl-pyridin-2-yl)-[1,2,4]oxadiazol-5-yl]-phenol (184): O21 N-Hydroxy-3-methyl-pyridine-2-carboxamidine and 3-methoxysalicylic acid were subjected to conditions outlined in Example 8 to produce 30 mg of desired product in 16% yield. [M H]⁺: 284.0; ¹H(CDCl₃) δ 8.67 (d, J=4.8 Hz, 1H), 7.71 (d, J=8.8 Hz, 1H), 7.65 (dd, J=8.0, 1.6 Hz, 1H), 7.39 (dd, J=8.0, 4.8 Hz, 1H), 7.11 (dd, J=8.0, 1.2 Hz, 1H), 7.00 (t, J=8.0 Hz), 3.97 (s,3H), 2.67 (s, 3H):

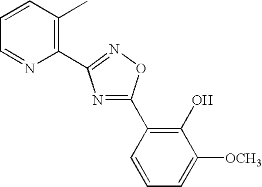

4-Bromo-2-[3-(3-methyl-pyridin-2-yl)-[1,2,4]oxadiazol-5-yl]-phenol (190):

O21 N-Hydroxy-3-methyl-pyridine-2-carboxamidine and 5-bromsalicylic acid were subjected to conditions outlined in Example 8 to produce 21 mg of desired product in 10% yield. [M H]⁺: 332.2, 334.0; ¹H(CDCl₃) δ 10.47 (s, 1H), 8.67 (d, J=4.0 Hz, 1H), 8.15 (d, J=2.0 Hz, 1H), 7.71 (d, J=7.6 Hz, 1H), 7.60 (dd, J=8.8, 2.8 Hz, 1H), 7.39 (dd, J=8.0, 4.8 Hz, 1H), 7.06 (d, J=8.8 Hz, 1H), 2.70 (s, 3H):

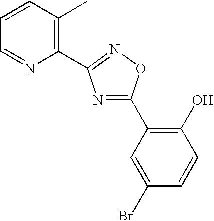

5-Methoxy-2-[3-(3-methyl-pyridin-2-yl)-[1,2,4]oxadiazol-5-yl]-phenol (175):

O21 N-Hydroxy-3-methyl-pyridine-2-carboxamidine and 4-methoxysalicylic acid were subjected to conditions outlined in Example 2 to produce 37 mg of desired product in 39% yield. [M+H]⁺: 284.0:

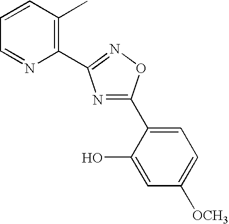

2-[5-(3-Methoxy-phenyl)-[1,2,4]oxadiazol-3-yl]-3-methyl-pyridine (176):

O21 N-Hydroxy-3-methyl-pyridine-2-carboxamidine and 3-methoxybenzoic acid were subjected to conditions outlined in Example 8 to produce 37 mg of desired product in 20% yield. [M+H]⁺: 268.1:

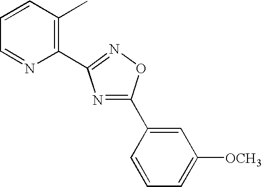

3-Methyl-2-(5-naphthalen-1-yl-[1,2,4]oxadiazol-3-yl)-pyridine (177):

O21 N-Hydroxy-3-methyl-pyridine-2-carboxamidine and 1-naphthoic acid were subjected to conditions outlined in Example 8 to produce 61 mg of desired product in 32% yield. [M+H]⁺: 288.0:

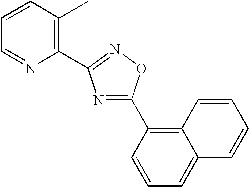

2-[5-(2,5-Dimethoxy-phenyl)-[1,2,4]oxadiazol-3-yl]-3-methyl-pyridine (178):

O21 N-Hydroxy-3-methyl-pyridine-2-carboxamidine and 2,5-dimethoxybenzoic acid were subjected to conditions outlined in Example 8 to produce 58 mg of desired product in 30% yield. [M+H]⁺: 298.2:

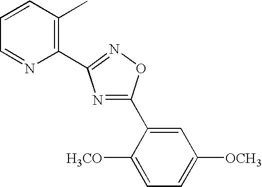

2-[3-(3-Methyl-pyridin-2-yl)-[1,2,4]oxadiazol-5-yl]-benzene-1,4-diol (179):

O21 N-Hydroxy-3-methyl-pyridine-2-carboxamidine and 2,5-dihydroxybenzoic acid were subjected to conditions outlined in Example 8 to produce 30 mg of desired product in 17% yield. [M+H]⁺: 270.1:

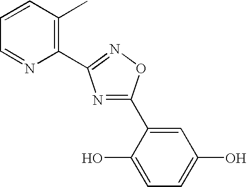

5-Dimethylamino-2-[3-(3-methyl-pyridin-2-yl)-t1,2,4]oxadiazol-5-yl]-phenol (180):

O21 N-Hydroxy-3-methyl-pyridine-2-carboxamidine and 4-dimethylaminosalicylic acid were subjected to conditions outlined in Example 8 to produce 13 mg of desired product in 7% yield. [M+H]⁺: 297.1:

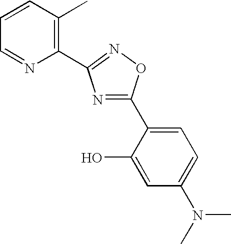

3-Methyl-2-15-(3-nitro-phenyl)-[1,2,4]oxadiazol-3-yl]-pyridine (181):

O21 N-Hydroxy-3-methyl-pyridine-2-carboxarnidine and 3-nitrobenzoic acid were subjected to conditions outlined in Example 8 to produce 39 mg of desired product in 21% yield. [M+H]⁺: 283.2:

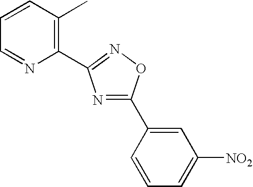

2-[5-(2-Chloro-4-nitro-phenyl)-[1,2,4]oxadiazol-3-yl]-3-methyl-pyridine (182):

O21 N-Hydroxy-3-methyl-pyridine-2-carboxamidine and 2-chlor-4-nitrobenzoic acid were subjected to conditions outlined in Example 8 to produce 51 mg of desired product in 24% yield. [M+H]⁺: 317.1:

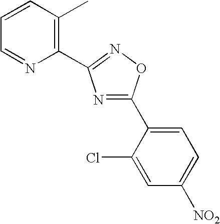

2-[5-(2-Methoxy-naphthalen-1-yl)-11,2,4]oxadiazol-3-yl]-3-methyl-pyridine (183):

O21 N-Hydroxy-3-methyl-pyridine-2-carboxamidine and 2-methoxy-1-naphthoic acid were subjected to conditions outlined in Example 8 to produce 9 mg of desired product in 4% yield. [M+H]⁺: 318.2:

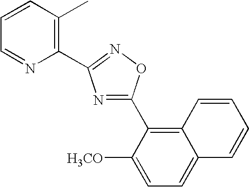

4-Chloro-2-[3-(3-methyl-pyridin-2-yl)-[1,2,4]oxadiazol-5-yl]-phenol (186):

O21 N-Hydroxy-3-methyl-pyridine-2-carboxamidine and 5-chlorosalicylic acid were subjected to conditions outlined in Example 8 to produce 25 mg of desired product in 13% yield. [M+H]⁺: 288.0:

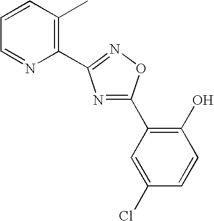

4-Fluoro-2-[3-(3-methyl-pyridin-2-yl)-[1,2,4]oxadiazol-5-yll -phenol (187):

O21 N-Hydroxy-3-methyl-pyridine-2-carboxamidine and 5-fluorosalicylic acid were subjected to conditions outlined in Example 8 to produce 24 mg of desired product in 13% yield. [M+H]⁺: 272.2:

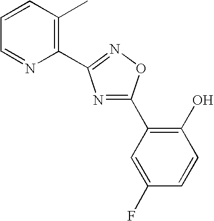

2-[3-(3-Methyl-pyridin-2-yl)-[1,2,4]oxadiazol-5-yl]-phenol (188):

O21 N-Hydroxy-3-methyl-pyridine-2-carboxamidine and salicylic acid were subjected to conditions outlined in Example 8 to produce 60 mg of desired product in 36% yield. [M+H]⁺: 254.0:

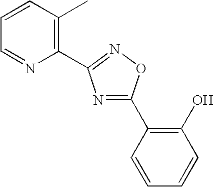

1-[3-(3-Methyl-pyridin-2-yl)-[1,2,4]oxadiazol-5-yl]-naphthalen-2-ol (189):

O21 N-Hydroxy-3-methyl-pyridine-2-carboxamidine and 2-hydroxy-1-naphthoic acid were subjected to conditions outlined in Example 8 to produce 23 mg of desired product in 11% yield. [M+H]⁺: 304.2:

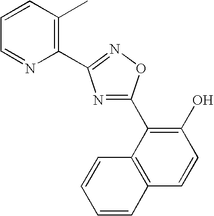

2-[5-(2-Chloro-5-nitro-phenyl)-[1,2,4]oxadiazol-3-yl]-3-methyl-pyridine (191):

O21 N-Hydroxy-3-methyl-pyridine-2-carboxamidine and 2-chloro-5-ntrobenzoic acid were subjected to conditions outlined in Example 8 to produce 44 mg of desired product in 21% yield. [M+H]⁺: 317.1:

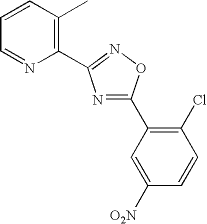

4-Methyl-2-[3-(3-methyl-pyridin-2-yl)-[1,2,4]oxadiazol-5-yl]-phenol (192):

O21 N-Hydroxy-3-methyl-pyridine-2-carboxamidine and 5-methylsalicylic acid were subjected to conditions outlined in Example 8 to produce 34 mg of desired product in 19% yield. [M+H]⁺: 268.2:

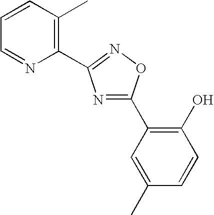

5-Methyl-2-[3-(3-methyl-pyridin-2-yl)-t1,2,4]oxadiazol-5-yl]-phenol (295):

O21 N-Hydroxy-3-methyl-pyridine-2-carboxamidine and 4-fluorosalicylic acid were subjected to conditions outlined in Example 8 to produce 11 mg of desired product in 6% yield. [M H]⁺: 268.3:

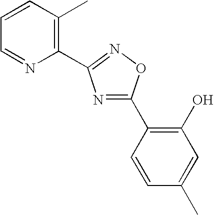

3,5-Dimethoxy-2-[3-(3-methyl-pyridin-2-yl)-[1,2,4]oxadiazol-5-yl]-phenol (240):

O21 N-Hydroxy-3-methyl-pyridine-2-carboxamidine and 4,6-dimethoxysalicylic acid were subjected to conditions outlined in Example 8 to produce 39 mg of desired product in 19% yield. [M H]⁺: 314.2; H NMR (CDCl₃) δ 11.80 (br s, 1H), 8.63 (dd, J=4.4, 1.2 Hz, 1H), 7.67 (dd, J=7.2, 1.6 Hz, 1H), 7.34 (dd, J=7.6, 4.4 Hz, 1H), 6.26 (d, J=2.0 Hz, 1H), 6.09 (d, J=2.8 Hz, 1H), 3.97 (s, 3H), 3.85 (s, 3H), 2.64 (s, 3H):

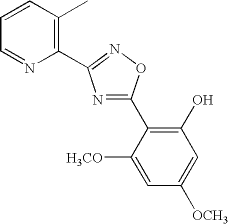

3-Methoxy-2-[3-(3-methyl-pyridin-2-yl)-[1,2,4]oxadiazol-5-yl]-phenol (241):

O21 N-Hydroxy-3-methyl-pyridine-2-carboxamidine and 6-methoxysalicylic acid were subjected to conditions outlined in Example 8 to produce 41 mg of desired product in 22% yield. [M H]⁺: 284.0; ¹H NMR (CDCl₃) δ 11.63 (s, 1H), 8.65 (dd, J=4.8, 1.2 Hz, 1H), 7.69 (d, J=7.6 Hz, 1H), 7.41 (t, J=8.4 Hz, 1H), 7.36 (dd, J=8.0, 4.8 Hz, 1H), 6.75 (dd, J=9.2, 0.8 Hz, 1H), 6.55 (d, J=8.4 Hz, 1H), 4.02 (s, 3H), 2.66 (s, 3H):

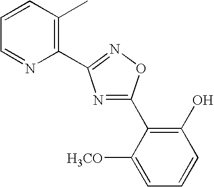

2-[3-(3-Methyl-pyridin-2-yl)-[1,2,4]oxadiazol-5-yl]-4-methylsulfanyl-phenol (294):

O21 N-Hydroxy-3-methyl-pyridine-2-carboxamidine and 5-(methylthio)salicylic acid were subjected to conditions outlined in Example 8 to produce 51 mg of desired product in 26% yield. [M H]⁺: 300.3 ; ¹H NMR (CDCl₃) δ 8.80 (s, 1H), 7.96 (d, J=2.4 Hz, 1H), 7.84 (d, J=7.2 Hz, 1H), 7.50 (m, 2H), 7.12 (d, J=8.8 Hz, 1H), 2.78 (s, 3H), 2.55 (s, 3H):

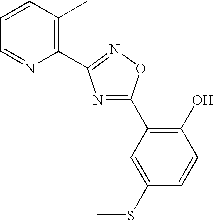

4-Bromo-2-methoxy-6-[3-(3-methyl-pyridin-2-yl)-[1,2,4]oxadiazol-5-yl]-phenol (242):

O21 N-Hydroxy-3-methyl-pyridine-2-carboxamidine and 5-bromo-3-methoxysalicylic acid were subjected to conditions outlined in Example 8 to produce 16 mg of desired product in 7% yield. [M H]⁺: 364.3, 365.3 ; ¹H NMR (CDCl₃) δ 10.60 (br s, 1H), 8.66 (d, J=4.8 Hz, 1H), 7.77 (d, J=2.4 Hz, 1H), 7.70 (d, J=7.6 Hz, 1H), 7.39 (dd, J=7.6, 4.8 Hz, 1H), 7.17 (d, J=2.0 Hz, 1H), 3.96 (s, 3H), 2.66 (s, 3H):

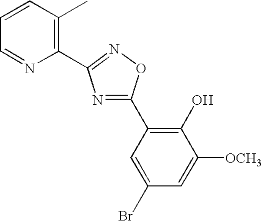

5-(2-Hydroxy-ethoxy)-2-[3-(3-methyl-pyridin-2-yl)-[1,2,4]oxadiazol-5-yl]-phenol (296):

O21 N-Hydroxy-3-methyl-pyridine-2-carboxamidine and 4-(2-hydroxyethoxy)salicylic acid were subjected to conditions outlined in Example 8 to produce 17 mg of desired product in 8% yield. [M H]⁺: 314.4; ¹H NMR (d₆-DMSO) δ 10.73 (s, 1H), 8.59 (d, J=3.2 Hz, 1H), 7.93 (d, J=8.4 Hz, 1H), 7.86 (d, J=7.6 Hz, 1H), 7.50 (dd, J=8.0, 4.8 Hz, 1H), 6.66 (m, 2H), 4.05 (t, J=4.4 Hz, 2H), 3.72 (m, 2H):

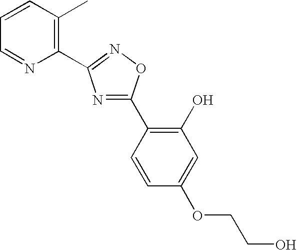

### EXAMPLE 14

Synthesis of 5-Benzyloxy-2-[3-(3-methyl-pYridin-2-yl)-1,2,41 oxadiazol-5-yl]-phenol (274).

Step 1: Preparation of O22 4-benzyloxysalicylic acid.

4-benzyloxysalicylaldehyde was subjected to the conditions outlined in example 281, step 1 to produce 970 mg of white powder in 72% yield.

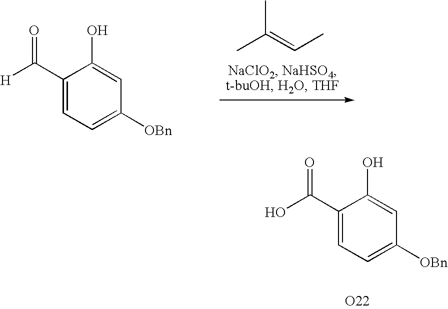

Step 2: Preparation of 5-Benzyloxy-2-[3-(3-methyl-pyridin-2-yl)-1,2,41 oxadiazol-5-yl]-phenol (274).

O21 N-Hydroxy-3-methyl-pyridine-2-carboxamidine and O22 4-benzyloxysalicylic acid were subjected to conditions outlined in Example 8 to produce 232 mg of desired product in 16% yield. [M H]⁺: 360.2; ¹H NMR (CDCl₃) δ 8.17 (dd, J=4.4, 1.2 Hz, 1H), 7.97 (d, J=8.8 Hz, 1H), 7.56 (d, J=8.0 Hz, 1H), 7.38 (m, 5H), 6.95 (dd, J=7.6, 5.2 Hz, 1H), 6.93 (dd, J=8.8, 2.4 Hz, 1H), 6.86 (d, J=2.0 Hz, 1H), 5.15 (s, 2H), 2.43 (s, 3H):

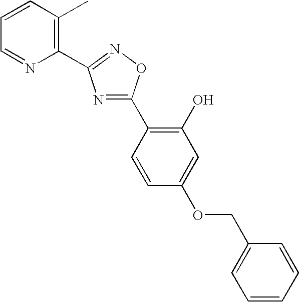

2-Methyl-6-[3-(3-methyl-pyridin-2-yl)-[1,2,4]oxadiazol-5-yl]-phenol (293):

O21 N-Hydroxy-3-methyl-pyridine-2-carboxamidine and 3-methylsalicylic acid were subjected to conditions outlined in Example 8 to produce 42 mg of desired product in 22% yield. [M H]⁺: 268.3; ¹H NMR (CDCl₃) δ 8.92 (d, J=4.8 Hz, 1H), 7.95 (d, J=7.6 Hz, 1H), 7.86 (dd, J=8.0, 0.8 Hz, 1H), 7.59 (dd, J=7.6, 5.2 Hz, 1H), 7.40 (d, J=7.6 Hz, 1HO, 6.94 (t, J=8.0 Hz, 1H), 2.81 (s, 3H), 2.37 (s, 3H):

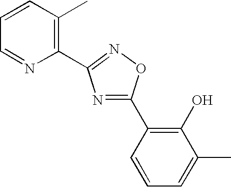

2,3-Dimethoxy-6-[3-(3-methyl-pyridin-2-yl)-[1,2,4]oxadiazol-5-yl]-phenol (282):

O21 N-Hydroxy-3-methyl-pyridine-2-carboxamidine and 3,4-dimethoxysalicylic acid were subjected to conditions outlined in Example 8 to produce 2 mg of desired product in 0.8% yield. [M H]⁺: 314.3; ¹H NMR (CDCl₃) δ 10.53 (br s, 1H), 8.67 (d, J=4.8 Hz, 1H), 7.78 (d, J=8.8 Hz, 1H), 7.70 (td, J=7.6, 0.8 Hz, 1H), 7.38 (dd, J=8.0, 4.8 hz, 1H), 6.67 (d, J=8.8 Hz, 1H), 3.98 (s, 3H), 3.96 (s,3H), 2.67 (s, 3H):

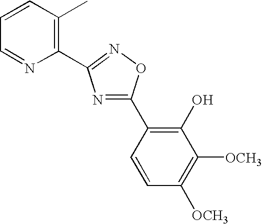

2,4-Dichloro-6-[3-(3-methyl-pyridin-2-yl)-[1,2,4]oxadiazol-5-yl]-phenol (278):

O18 3,5-dichlorosalicylic acid and O21 N-Hydroxy-3-methyl-pyridine-2-carboxamidine were subjected to protocol outlined in Example 8 to produce 23 mg of desired product in 15% yield. [M H]⁺: 322.2, 324.2; ¹H (CDCl₃) δ 8.66 (d, J=4.4 Hz, 1H), 7.94 (d, J=2.4 Hz, 1H), 7,72 (d, J=7.2 Hz, !H), 7.61 (d, J=2.0 1Hz, 1H), 7.40 (dd, J=7.6, 4.8 Hz, 1H):

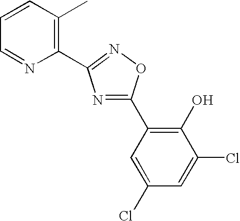

### EXAMPLE 15

Synthesis of 2-Bromo-4-chloro-6-[3-(3-methyl-pyridin-2-yl)-[1 ,2,41oxadiazol-5-yl]-phenol (279)

Step 1: Preparation of O23 3-bromo-5-chlorosalicylic acid.

3-bromo-5chlorosalicylaldehyde was subjected to the protocol outlined in example 10 to produce 160 mg of beige powder in 75% yield. [M H]⁺: 249.2, 251.1; ¹H (d₆-DMSO) δ 7.92 (dd, J=2.4, 1.2 Hz, 1H), 7.74 (dd, J=2.8, 1.2 Hz, 1H):

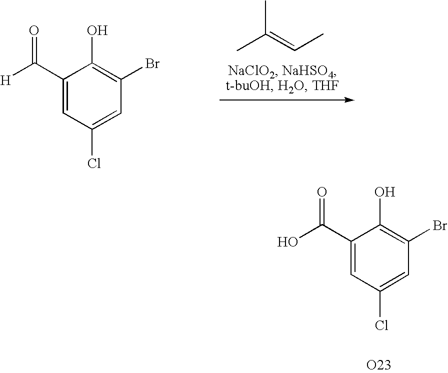

Step 2: Preparation of 2-Bromo-4-chloro-6-13-(3-methyl-pyridin-2-yl)-11.2,41oxadiazol-5-yl]-phenol (279)

O23 3-bromo-5-chlorosalicylic acid and O21 N-Hydroxy-3-methyl-pyridine-2-carboxamidine using protocol outlined in Example 8 to produce a yellow powder in 7% yield. [M H]⁺: 366.2, 368.2; ¹H (CDCl₃) δ 8.66 (dd, J=4.4, 1.2 Hz, 1H), 7.99 (d, J=2.4 Hz, 1H), 7.78 (d, J=2.8 Hz, 1H), 7.72 (dd, J=7.6, 0.8 Hz, 1H), 7.40 (dd, J=8.0, 4.8 Hz, 1H):

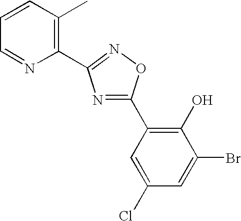

2,4-Dibromo-6-[3-(3-methyl-pyridin-2-yl)-[1,2,4]oxadiazol-5-yl]-phenol (280):

3,5-dibromosalicylic acid and O21 N-Hydroxy-3-methyl-pyridine-2-carboxamidine were subjected to conditions outlined in example 8 to produce 10 mg of yellow powder in 6% yield. [M H]⁺: 410.0, 412.0, 414.0; ¹H (CDCl₃) δ 8.66 (d, J=4.4 Hz, 1H), 8.13 (d, J=2.0 Hz, 1H), 7.91 (d, J=2.4 Hz, 1H), 7.71 (d, J=7.6 Hz, 1H), 7.40 (dd, J=7.6, 4.4 Hz, 1H), 2.64 (s, 3H):

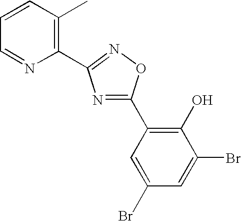

2-Isopropyl-6-[3-(3-methyl-pyridin-2-yl)-[1 ,2,4]oxadiazol-5-yl]-phenol (300):

3-isopropyl-2 hydroxybenzoic acid and O21 N-Hydroxy-3-methyl-pyridine-2-carboxamidine were subjected to conditions outlined in example 8 to produce 9 mg of yellow powder in 5% yield. [M H]⁺: 296.3; ¹H NMR (CDCl₃) δ 8.46 (d, J=4.8 Hz, 1H), 7.78 (dd, J=8.0, 2.0, 1H), 7.65 (td, J=7.6, 0.8 Hz, 1H), 7.44 (dd, J=7.6, 2.0 Hz, 1H), 7.31 (dd, J=7.6, 4.4 Hz, 1H), 6.89 (t, J=7.6 Hz, 1H), 3.40 (spt, J=6.8 Hz, 1H), 1.27 (d, J=6.8 Hz, 6H):

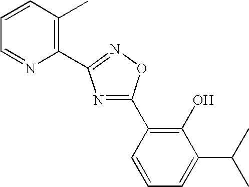

5-Ethoxy-2-[3-(3-methyl-pyridin-2-yl)-[1,2,4]oxadiazol-5-yl]-phenol (243):

4-Ethoxysalicylic acid and O21 N-Hydroxy-3-methyl-pyridine-2-carboxamidine were subjected to conditions outlined in example 8 to produce 17 mg of yellow-brown powder in 9% yield. [M H]⁺: 298.1; ¹H NMR (CDCl₃) δ 10.56 (s, 1H), 8.65 (dd, J=4.8, 1.2 Hz, 1H), 7.90 (d, J=9.2 Hz, 1H), 7.69 (d, J=8.0 Hz, 1H), 7.36 (dd, J=7.6, 4.8 Hz, 1H), 6.60 (m, 2H), 4.10 (q, J=7.2 Hz, 2H), 2.66 (s, 3H), 1.46 (t, J=7.2 Hz, 3H):

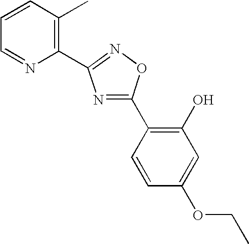

### EXAMPLE 16

Synthesis of 2-Ethoxy-6-[3-(3-methyl-pyridin-2-yl)-[1,2,4]oxadiazol-5-yl]-phenol (268)

Step 1: Preparation of O24 3-ethoxysalicylic acid

Oxidation of 3-ethoxysalicylaldehyde was carried out using protocol outlined in example 10 to yield 200 mg of desired product O24 in 94% yield. ¹H NMR (d₆-DMSO) δ 7.20 (dd, J=8.0, 1.6 Hz, 1H), 7.16 (dd, J=8.0, 1.6 Hz), 7.099 (t, J=8.0 Hz, 1H), 4.08 (q, J=6.8 Hz, 1H), 1.36 (t, J=6.8 Hz, 1H):

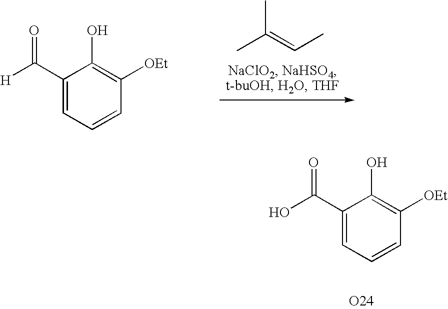

Step 2: Preparation of 2-Ethoxy-6-[3-(3-methyl-pyridin-2-yl)-[ 1,2,41 oxadiazol-5-yl]-phenol (268)

O24 2-ethoxysalyclic acid and O21 N-Hydroxy-3-methyl-pyridine-2-carboxamidine were subjected to protocol outlined in example 8 to produce 2.8 mg of a white solid in 24% yield. [M H]⁺: 298.2; ¹H NMR (CDCl₃) δ 10.54 (s, 1H), 8.66 (d, J=4.4 Hz, 1H), 7.70 (d, J=8.8 Hz, 1H), 7.65 (dd, J=8.0, 1.6 Hz, 1H), 7.38 (dd, J=8.4, 4.8 Hz, 1H), 7.12 (d, J=7.6 Hz, 1H), 6.98 (t, J=8.0 Hz, 1H), 4.18 (q, J=7.2 Hz, 1H), 1.23 (t, J=7.2 Hz, 1H):

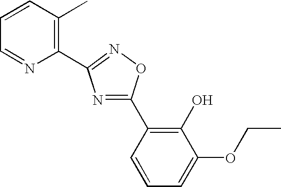

### EXAMPLE 17

Synthesis of 4-Benzyloxy-3-[3-(3-methyl-pyridin-2-yl)-[1,2,4loxadiazol-5-yl]-phenol (267)

Step 1: Preparation of [O25] 2-Benzyloxy-5-hydroxy-benzaldehyde 2,5-dihydroxybenzaldehyde (2.14 g, 15.5 mmoles) was dissolved in dry N,N-dimethylformamide (60 ml) under argon. Cesium Carbonate (2.0 equiv, 10.1 g, 31 mmoles) and Benzyl Bromide (1.5 equiv, 2.7 ml, 23.2 mmoles) wete then added. The mixture stirred at ambient temperature for 15 hours and was then concentrated in vacuo. The residue was then partitioned in ethyl acetate and 0.1 N HCL. The organic layer was dried with magnesium sulfate, filtered, and purified by chromatography to produce 1.2 g of a red-brown solid in 34% yield. R_{f}: 0.43 (9:1 CHCl₃/THF); ¹H NMR (CDCl₃) δ 10.35 (s, 1H), 7.918 (s, 1H), 7.28 (m, 6H), 7.00 (dd, J=9.6, 3.2 Hz, 1H), 6.85 (d, J=9.2 Hz), 5.03 (s, 2H):

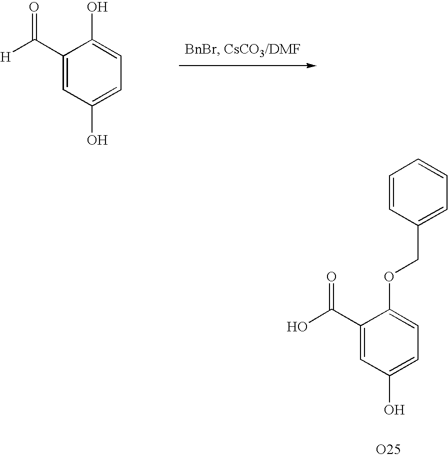

Step2: Preparation of O26 2-Benzyloxy-5-hydroxy-benzoic acid

Synthesis was carried out using 2-Benzyloxy-5-hydroxy-benzaldehyde O25 and protocol outlined in example 10 to yield 433 mg of white powder in 81% yield. R_{f}: 0.17 (9:1 CHCl₃/THF); [M H]⁺: 245.2; ¹H NMR (CDCl₃) δ 7.77 (d, J=3.2 Hz, 1H), 7.40 (m, 5H), 7.09 (dd, J=8.8, 3.2 Hz, 1H), 7.00 (d, J=8.8 Hz, 1H), 5.21 (s, 2H):

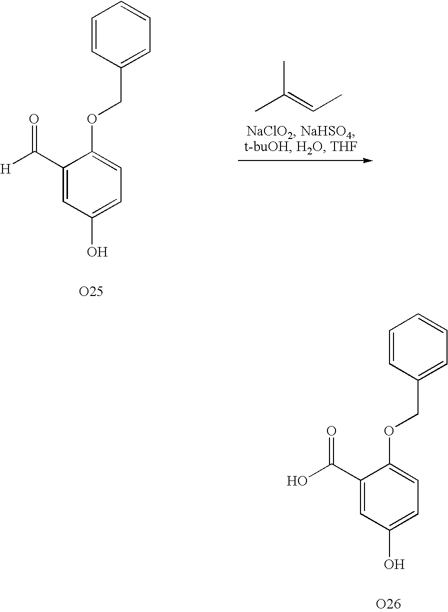

Step3: Preparation of 4-Benzyloxy-3-13-(3-methyl-pyridin-2-yl)-11,2,41 oxadiazol-5-yl]-phenol (267)

Synthesis was carried out with 2-Benzyloxy-5-hydroxy-benzoic acid O26 and N-Hydroxy-3-methyl-pyridine-2-carboxamidine O21 were subjected to protocol outlined in example 8 to produce 103 mg of white powder in 14% yield. [M H]⁺: 360.3; ¹H NMR (CDCl₃) δ 8.61 (s, 1H), 8.57 (d, J=3.6 Hz, 1H), 7.64 (d, J=2.8 Hz., 1H), 7.60 (d, J=8.0, 1H), 7.47 (d, J=7.6 Hz, 1H), 7.26 (m, 5H), 6.97 (dd, J=8.8, 2.8 Hz, 1H), 6.91 (d, J=8.8 Hz, 1H), 5.12 (s, 2H), 2.58 (s, 3H):

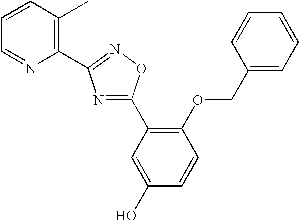

### EXAMPLE 18

Synthesis of 4-Ethoxy-2-[3-(3-methyl-pyridin-2-yl)-[1,2,4oxadiazol-5-yl]-phenol (271)

Step 1: Preparation of O27 2-15-[5-Ethoxy-2-(2-methylene-pent-3-enyloxy)-phenyyl]-[1,2 4]oxadiazol-3-yl]-3-methyl-pyridine

4-Benzyloxy-3-[3-(3-methyl-pyridin-2-yl)-[1,2,4]oxadiazol-5-yl]-phenol (267) (33 mg, 0.09 mmoles) was dissolved in acetonitrile (0.2 ml). Potassium carbonate (1.5 equiv, 18 mg, 0.13 mmoles) and ethyl bromide (1.5 equiv, 10 μl, 0.13 mmoles), and 18-crown-6 (1.5 equiv, 34 mg, 0.13 mmoles) were then added. The mixture stirred at ambient temperature for 6 h. The crude material was then purified by chromatography to produce 23 mg of a clear oily residue in 66% yield. R_{f}: 0.52 (20:1 CH₂Cl₂/MeOH); ¹H NMR (CDCl₃) δ 8.68 (dt, J=4.8, 0.8 Hz, 1H), 7.77 (d, J=2.8 Hz, 1H), 7.67 (d, J=7.6 Hz, 1H), 7.54, (d, J=7.2 Hz, 1H), 7.34 (m, 5H), 7.05 (m, 2H), 5.28 (s, 2H), 4.06 (q, J=6.8 Hz, 2H), 2.68 (s, 3H), 1.41 (t, J=6.8 Hz, 3H):

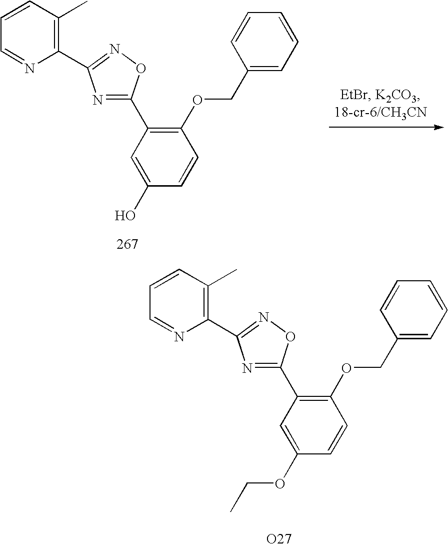

Step 2: Preparation of 4-Ethoxy-2-[3-(3-methyl-pyridin-2-yl)-[1,2,4]oxadiazol-5-yl]-phenol (271)

O27 2-[5-(2-Benzyloxy-5-ethoxy-phenyl)-[1,2,4]oxadiazol-3-yl]-3-methyl-pyridine (23 mg, 0.06 mmoles) was dissolved in chloroform (300 el). lodotrimethylsilane (3.0 equiv, 29 μl, 0.18 mmoles) was then added and the reaction stirred for 2 h at 50° C. The reaction was quenched with the addition of methanol (1 ml). The mixture was then dried in vacuo, and purified by chromatography to produce 18 mg of a yellow powder in quantitative yield. R_{f}: 0.25 (20:1 CH₂Cl₂/MeOH); ¹H NMR (CDCl₃) δ 10.04 (s, 1H), 8.65 (dd, J=4.8, 1.2 Hz, 1H), 7.70 (dq, J=8.0, 0.8 Hz), 7.45 (d, J=2.8 Hz, 1H), 7.37 (dd, J=7.6, 4.4 Hz, 1H), 7.13 (dd, J=8.8, 2.8 Hz, 1H), 7.06 (d, J=8.8 Hz, 1H), 4.07 (q, J=7.2 Hz, 2H), 2.66 (s, 3H), 1.45 (t, 7.2 Hz, 3H):

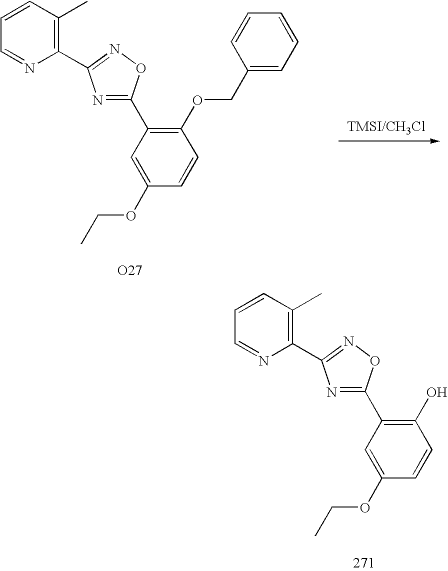

2-[3-(3-Methyl-pyridin-2-yl)-[1,2,4]oxadiazol-5-yl]-4-propoxy-phenol (272):

4-Benzyloxy-3-[3-(3-methyl-pyridin-2-yl)-[1,2,4]oxadiazol-5-yl]-phenol (267) was alkylated with propylbromide, and then debenzylated using protocol as outlined in example 18 to produce 7.5 mg of a yellow solid in 24% yield. R_{f}: 0.22 (20:1 CH₂Cl₂/MeOH); ¹H NMR (CDCl₃) δ 10.04 (s, 1H), 8.65 (dd, J=4.8, 1.6 Hz, 1H), 7.70 (dd, J=8.8, 0.8 Hz, 1H), 7.46 (d, J=3.2 Hz, 1H), 7.37 (dd, J=7.6, 4.4 Hz), 7.13 (dd, , J=9.2, 2.8 Hz, 1H), 3.96 (t, J=6.8 Hz, 2H), 2.66 (s, 3H), 1.84 (sx, J=7.2 Hz, 2H), 1.08 (t, J=7.2 Hz, 3H):

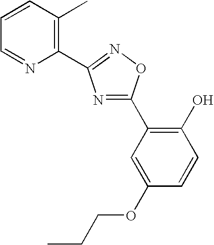

2-[3-(3-Methyl-pyridin-2-yl)-[1,2,4]oxadiazol-5-yl]-4-phenethyloxy-phenol (273):

4-Benzyloxy-3-[3-(3-methyl-pyridin-2-yl)-[1,2,4]oxadiazol-5-yl]-phenol (267) was alkylated with 2-(bromoethyl)benzene and then debenzylated using protocol outlined in example 18 to produce 12 mg of yellow powder in 38% yield. R_{f}: 0.12 (20:1 CH₂Cl₂/MeOH); [M H]⁺: 374.2; ¹H NMR (CDCl₃) δ 10.04 (s, 1H), 8.65 (d, J=4.4, 1H), 7.69 (dd, J=7.6, 0.8 Hz, 1H), 7.45 (d, J=2.8 Hz, 1H), 7.37 (dd, J=7.6, 4.8, 1H), 7.31 (m,5H), 7.19 (dd, J=8.8, 2.8 Hz, 1H), 4.22 (t, J=6.8 Hz, 2H), 3.70 (s, 3H), 3.13 (t, 2H):

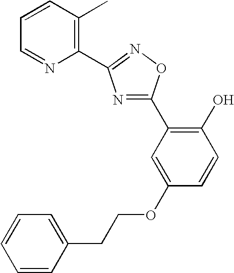

### EXAMPLE 19

Synthesis of 12-Hydroxy-3-[3-(3-methyl-pyridin-2-yl)-[ 1,2,4]oxadiazol-5-yl]-phenyl}-carbamic acid tert-butyl ester (298)

Step 1: Preparation of O28 3-tert-Butoxycarbonylamino-2-hydroxy-benzoic acid

3-aminosalicylic acid (1.0 g, 6.5 mmoles) was dissolved in dry tetrahydrofuran (50 ml) under argon. Diisopropylethylamine (2.0 equiv, 2.3 ml, 13.0 mmoles) and di-t-butyldicarbonate (1.5 equiv, 2. 1 g, 9.7 mmoles) were added. The reaction stirred at ambient temperature for 18 h. The mixture was then concentrated in vacuo, diluted in ethyl acetate and partitioned with 0.1 N aqueous HCl. The organic layer was dried over magnesium sulfate, filtered, and purified by chromatography. 770 mg of a grey-white solid was isolated in 47% yield. R_{f}: 0.26 (20:1 CH₂Cl₂/MeOH); [M]⁻: 252.3; ¹H NMR (d₆-DMSO) δ 8.02 (s, 1H), 7.82 (d, J=8.0 Hz, 1H), 7.48 (dd, J=8.0, 1.6 Hz, 1H), 6.85 (t, J=8.0, 1H), 1.45 (s, 9H):

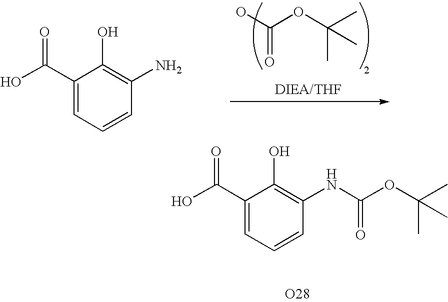

Step 2: Preparation of O12-Hydroxy-3-[3-(3-methyl-pyridin-2-yl)-[1,2,4]oxadiazol-5-yl]-phenyl]-carbamic acid tert-butyl ester (298)

Synthesis was carried out with 3-tert-Butoxycarbonylamino-2-hydroxy-benzoic acid O28 and N-Hydroxy-3-methyl-pyridine-2-carboxamidine O21 using protocol outlined in example 8 to produce 123 mg of white powder in 40% yield. [M H]⁺: 369.3; ¹H NMR (CDCl₃) δ 8.67(dt, J=4.4, 0.8 Hz, 1H), 8.34 (d, J=7.6 Hz, 1H), 7.70 (dt, J=6.8, 0.8 Hz, 1H), 7.67 (dd, J=8.0, 1.2 Hz, 1H), 7.39 (dd, J=7.6, 4.8 Hz, 1H), 7.24 (b, 1H), 7.038 (t, J=8.0 Hz, 1H), 2.65 (s, 3H), 1.57 (s, 9H):

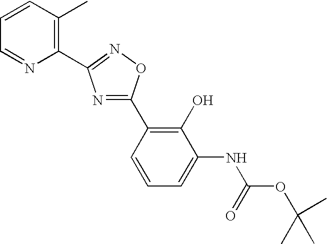

{3-Hydroxy-4-[3-(3-methyl-pyridin-2-yl)-[1,2,4]oxadiazol-5-yl]-phenyl}-carbamic acid tert-butyl ester (276):

The synthesis was carried out with BOC protected 4-aminosalicylic acid and and N-Hydroxy-3-methyl-pyridine-2-carboxamidine using protocol outlined in example 19 to produce 170 mg of white powder in 25% yield. R_{f}: 0.24 (9:1 CHCl₃/THF); [M H]⁺: 369.3; ¹H NMR (CDCl₃) δ 10.44 (s, 1H), 8.63 (s,1H), 7.88 (d, J=8.8 Hz, 1H), 7.68 (d, J=7.2 Hz, 1H), 7.35 (s, 1H), 7.21 (s, 1H), 7.04 (d, J=8.4 Hz, 1H), 6.87 (s, 1H), 2.64 (s, 3H), 1.52 (s, 9H).

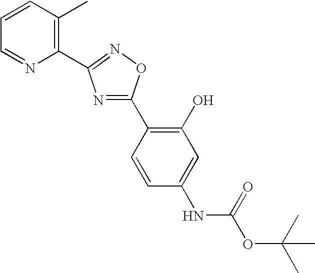

### EXAMPLE 20

Synthesis of 5-Amino-2-13-(3-methyl-pyridin-2-yl)-[1.2,41oxadiazol-5-yl]-phenol (277)

{3-Hydroxy-4-[3-(3-methyl-pyridin-2-yl)-[ 1,2,4]oxadiazol-5-yl]-phenyl}-carbamic acid tert-butyl ester (276) (134 mg, 0.36 mmoles) was dissolved in dichloromethane (500 μl). Trifluoroacetic acid (500 μl) was then added. The mixture stirred for 30 m at ambient temperature, and was then diluted in dichloromethane, and partitioned with aqueous 5% sodium bicarbonate. The organic layer was dried over magnesium sulfate, filtered, and dried in vacuo to produce 125 mg the TFA salt as a white powder in quantitative yield. R_{f}: 0.40 (9:1 CH₂Cl₂/MeOH); [M H]⁺: 269.2; ¹H NMR (d₆-DMSO) δ 10.30 (s, 1H), 8.58 (d, J=2.4 Hz, 1H), 7.64 (d, J=8.8 Hz, 1H), 7.49 (dd, J=7.6, 4.8 Hz, 1H), 6.26 (dd, J=8.8, 2.0 Hz, 1H), 6.17 (s, 3H), 2.48 (s, 3H):

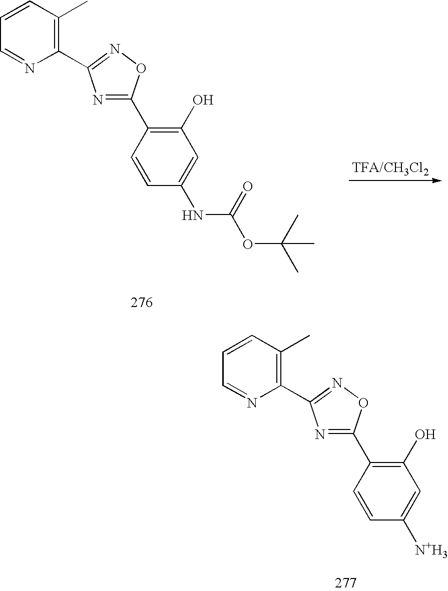

### EXAMPLE 21

Synthesis of N-{3-Hydroxy-4-[3-(3-methyl-pyridin-2-yl)-[1,2,41oxadiazol-5-yl]-phenyl}-acetamide (291)

5-Amino-2-[3-(3-methyl-pyridin-2-yl)-[1,2,4]oxadiazol-5-yl]-phenol (277) (25 mg, 0.09 mmoles) was dissolved in dry N,N-dimethylformamide (250 μl). Acetic anhydride (1.0 equiv, 9 μl, 0.09 mmoles) and diisopropylethylamine (2.0 equiv, 31 μl, 0.18 mmoles) were then added, and the reaction stirred for 15 hours at ambient temperature. The crude mixture was dried in vacuo, and purified by chromatography to produce 8 mg of white powder in 28% yield. [M H]⁺: 311.3; ¹H NMR (CDCl₃) δ 8.64 (d, J=4.4 Hz, 1H), 8.13 (d, J=8.8 Hz, 1H). 7.66 (d, J=7.6 Hz, 1H), 7.32 (dd, J=7.6, 4.8 Hz, 1H), 6.62 (dd, J=8.8, 2.0 Hz, 1H), 6.44 (d, J=2 Hz, 1H), 2.68 (s, 3H), 2.48 (s, 3H):

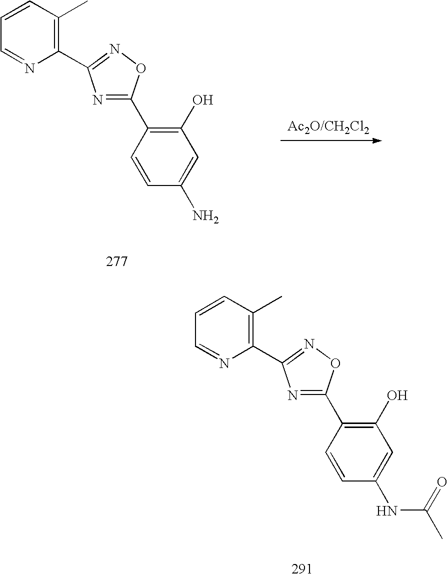

### EXAMPLE 22

Synthesis of N-{3-Hydroxy-4-[3-(3-methyl-pyridin-2-yl)-[1,2,4oxadiazol-5-yl]-phenyl}-benzamide (289)

5-Amino-2-[3-(3-methyl-pyridin-2-yl)-[1,2,4]oxadiazol-5-yl]-phenol (277) (25 mg, 0.09 mmoles) was dissolved in CH₂Cl₂ (250 μl) and cooled to 0° C. Benzoyl chloride (1.0 equiv, 10 μl, 0.09 mmoles) and diisopropylethylamine (2 equiv, 31 μl, 0.18 mmoles) were added, and stirred for 1 h with warming to ambient temperature. The crude mixture was then dried in vacuo, and purified by chromatography to produce 5 mg of white powder in 12% yield. [M H]⁺: 373.3; ¹H NMR (CDCl₃) δ 8.72 (d, J=4.0, 1H), 8.28 (dd, J=8.0, 1.2 Hz, 1H), 8.20 (d, J=8.8 Hz, 1H), 7.75 (d, J=8.0, 1H), 7.65 (t, J=7.2 Hz, 1H), 7.52 (t, J=8.0 Hz, 2H), 7.44, (dd, J=7.6, 5.2 Hz, 1H), 6.69 (dd, J=8.8, 2.0 Hz, 1H), 6.60 (d, J=2.0 Hz, 1H), 2.50 (s, 1H).

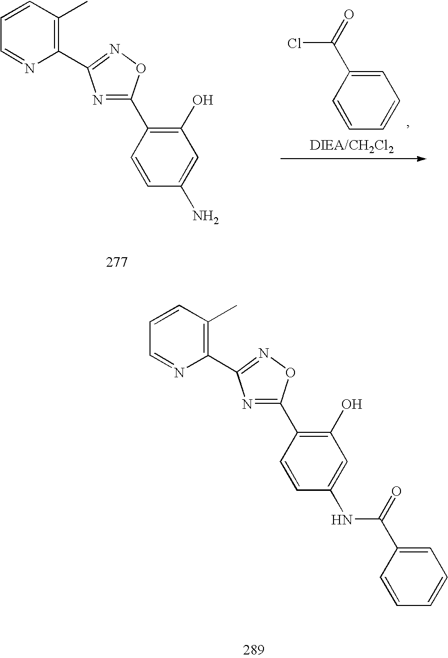

### EXAMPLE 23

Synthesis of 5-Benzylamino-2-[3-(3-methyl-pyridin-2-yl)-[1,2,4]oxadiazol-5-yl]-phenol (288)

5-Amino-2-[3-(3-methyl-pyridin-2-yl)-[1,2,4]oxadiazol-5-yl]-phenol (277) (25 mg, 0.09 mmoles) and benzaldehyde (5 equiv, 46 μl, 0.45 mmoles) were dissolved in methanol (2 ml) and glacial acetic acid (100 μl). Sodium borohydride (1.0 equiv, 4 mg, 0.09 mmoles) was then added with stirring at ambient temperature for 1 h. The mixture was concentrated in vacuo and purified by chromatography to prduce 8 mg of yellow powder in 25% yield. [M H]⁺: 359.3 ; ¹H NMR (d₆-DMSO) δ 10.35 (s, 1H), 8.58 (s, 1H), 7.83 (m, 1H), 7.66 (d, J=8.8 Hz, 1H), 7.48 (m, 1H), 7.32 (m,4H ), 7.23 (m, 1H), 6.37 (d, J=8.4 Hz, 1H), 6.15 (s, 1H), 5.72 (s, 1H), 5.69 (s, 1H), 4.34 (s, 2H), 2.52 (s, 3H):

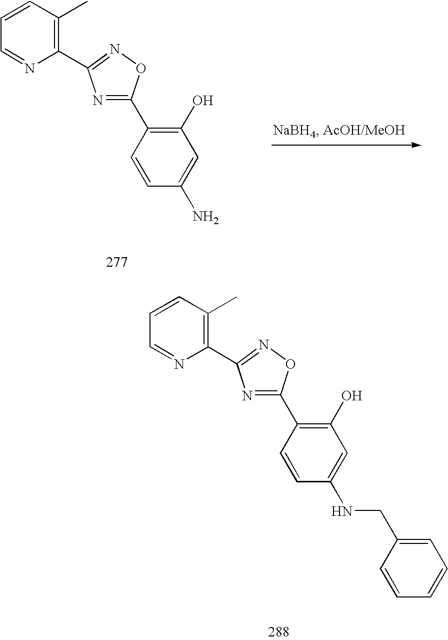

{4-Hydroxy-3-[3-(3-methyl-pyridin-2-yl)-[1,2,4]oxadiazol-5-yl]-phenyl}-carbamic acid tert-butyl ester (266):

The synthesis was carried out with BOC protected 4-aminosalicylic acid and and O21 N-Hydroxy-3-methyl-pyridine-2-carboxamidine using protocol outlined in example 19 to produce 305 mg of clear crystals in 31% yield. [M H]⁺: 369.4; ¹H NMR (CDCl₃) δ 8.65 (dd, , J=4.0, 0.8 Hz, 1H), 8.04 (d, J=2.4 Hz, 1H), 7.69 (dq, J=7.6, 0.8 Hz, 1H), 7.46 (d, J=7.2 Hz, 1H), 7.37 (dd, J=7.6, 4.8 Hz, 1H), 7.06 (d, J=8.8 Hz, 1H), 6.57 (b, 1H), 2.63 (s, 3H), 1.55 (s, 9H):

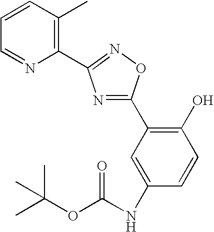

4-Amino-2-[3-(3-methyl-pyridin-2-yl)-[1,2,4]oxadiazol-5-yl]-phenol (270):

Deprotection of {4-Hydroxy-3-[3-(3-methyl-pyridin-2-yl)-[ 1 ,2,4]oxadiazol-5-yl]-phenyl}-carbamic acid tert-butyl ester was carried out using the protocol outlined in example 20 to produce 535 mg of white powder as the TFA salt in quantitative yield. [M H]⁺: 269.3; ¹H NMR (CDCl₃) δ 8.50 (dd, J=4.8, 1.2 Hz, 1H), 7.67 (d, J=2.4 Hz, 1H), 7.60 (dd, J=8.0, 0.8 Hz, 1H), 7.25 (m, 2H), 6.94 (d, J=8.8 Hz, 1H), 2.52 (s, 3H):

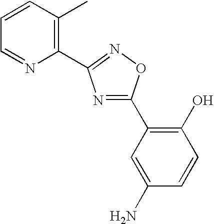

N-{4-Hydroxy-3-[3-(3-methyl-pyridin-2-yl)-[1,2,4]oxadiazol-5-yl]-phenyl}-acetamide (275):

The synthesis was carried out using 4-Amino-2-[3-(3-methyl-pyridin-2-yl)-[1,2,4]oxadiazol-5-yl]-phenol and the protocol outlined in example 21 to produce 21 mg of a yellow oily solid in 84% yield. [M H]⁺: 311.3; ¹H NMR (CDCl₃) o 8.64 (b, 1H), 8.06 (b, 1H), 7.68 (d, J=7.2 Hz, 1H), 7.53 (d, J=10.0 Hz, 1H), 7.35 (m, 1H), 7.11 (s, 1H), 7.02 (d, J=8.8 Hz, 1H), 2.608 (s, 3H), 2.128 (s, 1H):

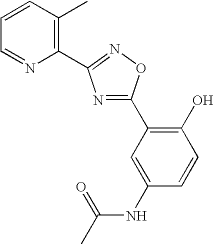

N-{4-Hydroxy-3-[3-(3-methyl-pyridin-2-yl)-[1,2,4]oxadiazol-5-yl]-phenyl}-benzamide (290):

The synthesis was carried out using 4-Amino-2-[3-(3-methyl-pyridin-2-yl)-[1,2,4]oxadiazol-5-yl]-phenol and the protocol outlined in example 22 to produce 4.5 mg of white powder in 12% yield. [M H]⁺: 373.3; ¹H NMR (CDCl₃) δ 8.76 (d, J=3.2 Hz, 1H), 8.33 (d, J=2.8Hz, 1H), 7.90 (t, J=6.8 Hz, 2H), 7.79 (t, J=8.0, 2H), 7.51 (m, 3H), 7.25 (s, 1H), 7.18 (d, J=9.2 Hz, 1H), 2.70 (s, 3H):

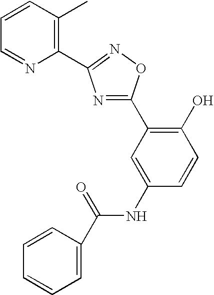

### EXAMPLE 24

Synthesis of [297] 4-(5,5-Dimethyl-[1,3]dioxan-2-yl)-2-[3-(3-methyl-pyridin-2-yl)-[1,2,4]oxadiazol-5-yl]-phenol

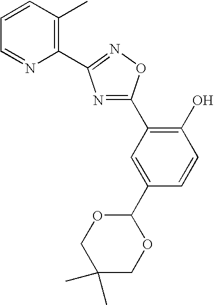

Step 1: Preparation of [O29] 5-(5,5-Dimethyl-[1,3]dioxan-2-yl)-2-hydroxy-benzoic acid

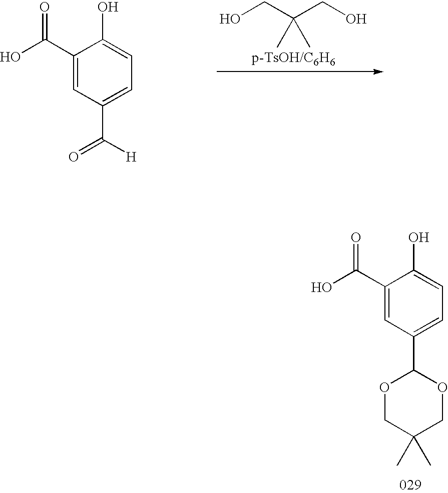

5-formylsalicylic acid (530 mg, 3.2 mmoles) and 2,2-dimethyl-1,3-propanediol (1.1 equiv, 366 mg, 3.5 mmoles) were dissolved in dry benzene (12 ml). P-toluenesulfonic acid (0.01 equiv, 6 mg, 0.03 mmoles) was then added, and the mixture was heated to reflux for 15 h in a flask fitted with a Dean-Stark trap and condenser. The mixture was then concentrated in vacuo and purified by chromatography to produce 782 mg of a white solid in 97% yield. [M]⁻: 251.4; ¹H NMR (d₆-DMSO) δ 7.82 (d, J=2.4 Hz, 1H), 7.52 (dd, J=8.4, 2.0 Hz, 1H), 6.919 (d, J=8.8 Hz, 1H), 5.36 (s, 1H), 3.62 (q, J=10.4 Hz, 4 H), 1.16 (s, 3H), 0.74 (s, 3H).

Step 2: Preparation of [2971 4-(5,5-Dimethyl-[],3]dioxan-2-yl)-2-[3-(3-methyl-pyridin-2-yl)-[1,2,4]oxadiazol-5-yl]-phenol

The synthesis was carried out with [O29] 5-(5,5-Dimethyl-[1,3]dioxan-2-yl)-2-hydroxy-benzoic acid and [O21]N-Hydroxy-3-methyl-pyridine-2-carboxamidine using protocol outlined in example 8 to produce 96 mg of a yellow solid in 48% yield. [M H]⁺: 368.4; 1H NMR (CDCl₃) δ 8.66 (dd, J=3.6, 1.6 Hz, 1H), 8.19 (d, J=2.4 Hz, 1H), 7.69 (m, 2H), 7.38 (dd, J=7.6, 4.4 Hz, 1H), 7.15 (d, J=8.8Hz, 1H), 5.42 (s, 1H), 3.81 (d, J=11.2 Hz, 2H), 3.69 (d, J=11.2 Hz, 2H), 2.67 (s, 3H), 1.33 (s, 3H), 0.84 (s, 3H).

### EXAMPLE 14

Synthesis of [274] 5-Benzyloxy-2-[3-(3-methyl-pyridin-2-yl)-[1,2,4]oxadiazol-5-yl]-phenol

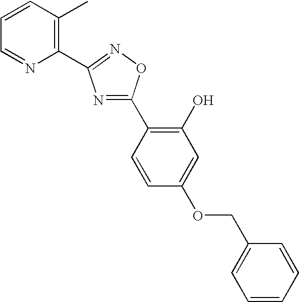

Step 1: Preparation of [O22] 4-benzyloxysalicylic acid

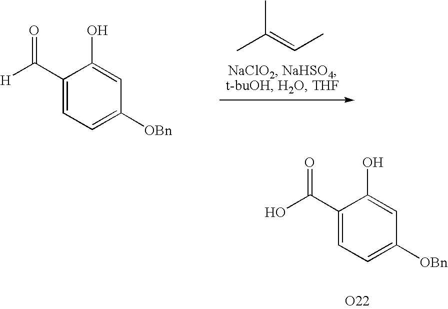

4-benzyloxysalicylaldehyde was subjected to the conditions outlined in example 281, step 1 to produce 970 mg of white powder in 72% yield.

Step 2: Preparation of [274] 5-Benzyloxy-2-[3-(3-methyl-pyridin-2-yl)-[1,2,4]oxadiazol-5-yl]-phenol

[O21]N-Hydroxy-3-methyl-pyridine-2-carboxamidine and [O22] 4-benzyloxysalicylic acid were subjected to conditions outlined in Example 8 to produce 232 mg of desired product in 16% yield. [M H]⁺: 360.2; ¹H NMR (CDCl₃) δ 8.17 (dd, J=4.4, 1.2 Hz, 1H), 7.97 (d, J=8.8 Hz, 1H), 7.56 (d, J=8.0 Hz, 1H), 7.38 (m, 5H), 6.95 (dd, J=7.6, 5.2 Hz, 1H), 6.93 (dd, J=8.8, 2.4 Hz, 1H), 6.86 (d, J=2.0 Hz, 1H), 5.15 (s, 2H), 2.43 (s, 3H).

293: 2-Methyl-6-[3-(3-methyl-pyridin-2-yl)-[1,2,4]oxadiazol-5-yl]-phenol

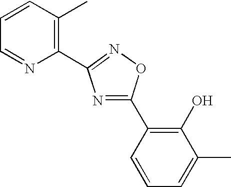

[O21]N-Hydroxy-3-methyl-pyridine-2-carboxamidine and 3-methylsalicylic acid were subjected to conditions outlined in Example 8 to produce 42 mg of desired product in 22% yield.

[M H]⁺: 268.3; ¹H NMR (CDCl₃) δ 8.92 (d, J=4.8 Hz, 1H), 7.95 (d, J=7.6 Hz, 1H), 7.86 (dd, J=8.0, 0.8 Hz, 1H), 7.59 (dd, J=7.6, 5.2 Hz, 1H), 7.40 (d, J=7.6 Hz, 1HO, 6.94 (t, J=8.0 Hz, 1H), 2.81 (s, 3H), 2.37 (s, 3H).

282: 2,3-Dimethoxy-6-[3-(3-methyl-pyridin-2-yl)-[1,2,4]oxadiazol-5-yl]-phenol

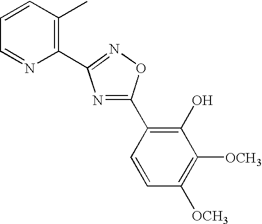

p 021] N-Hydroxy-3-methyl-pyridine-2-carboxamidine and 3,4-dimethoxysalicylic acid were subjected to conditions outlined in Example 8 to produce 2 mg of desired product in 0.8% yield. [M H]⁺: 314.3; ¹H NMR (CDCl₃) δ 10.53 (br s, 1H), 8.67 (d, J=4.8 Hz, 1H), 7.78 (d, J=8.8 Hz, 1H), 7.70 (td, J=7.6, 0.8 Hz, 1H), 7.38 (dd, J=8.0, 4.8 hz, 1H), 6.67 (d, J=8.8 Hz, 1H), 3.98 (s, 3H), 3.96 (s,3H), 2.67 (s, 3H).

278: 2,4-Dichloro-6-[3-(3-methyl-pyridin-2-yl)-[1,2,4]oxadiazol-5-yl]-phenol

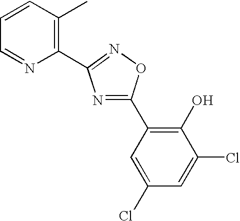

[O18] 3,5-dichlorosalicylic acid and [O21]N-Hydroxy-3-methyl-pyridine-2-carboxamidine were subjected to protocol outlined in Example 8 to produce 23 mg of desired product in 15% yield. [M H]⁺: 322.2, 324.2; ¹H (CDCl₃) δ 8.66 (d, J=4.4 Hz, 1H), 7.94 (d, J=2.4 Hz, 1H), 7,72 (d, J=7.2 Hz, !H), 7.61 (d, J=2.0 1Hz, 1H), 7.40 (dd, J=7.6, 4.8 Hz, 1H).

### EXAMPLE 15

Synthesis of [279] 2-Bromo-4-chloro-6-[3-(3-methyl-pyridin-2-yl)-[1,2,4]oxadiazol-5-yl]-phenol

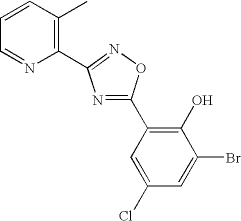

Step 1: Preparation of [O23] 3-bromo-5-chlorosalicylic acid

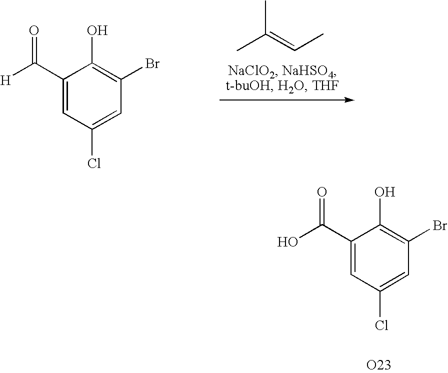

3-bromo-5chlorosalicylaldehyde was subjected to the protocol outlined in example 10 to produce 5 160 mg of beige powder in 75% yield. [M H]⁺: 249.2, 251.1; 1H (d₆-DMSO) δ 7.92 (dd, J=2.4, 1.2 Hz, 1H), 7.74 (dd, J=2.8, 1.2 Hz, 1H).

Step 2: Preparation of [279]2-Bromo-4-chloro-6-[3-(3-methyl-pyridin-2-yl)-[1, 2,4]oxadiazol-5-yl]-phenol

[O23]3-bromo-5-chlorosalicylic acid and [O21]N-Hydroxy-3-methyl-pyridine-2-carboxamidine using protocol outlined in Example 8 to produce a yellow powder in 7% yield. [M H]⁺: 366.2, 368.2; ¹H (CDCl₃) δ 8.66 (dd, J=4.4, 1.2 Hz, 1H), 7.99 (d, J=2.4 Hz, 1H), 7.78 (d, J=2.8 Hz, 1H), 7.72 (dd, J=7.6, 0.8 Hz, 1H), 7.40 (dd, J=8.0, 4.8 Hz, 1H).

Compound 280: 2,4-Dibromo-6-[3-(3-methyl-pyridin-2-yl)-[1,2,4]oxadiazol-5-yl]-phenol

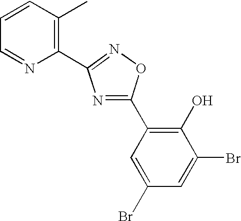

3,5-dibromosalicylic acid and [O21] N-Hydroxy-3-methyl-pyridine-2-carboxamidine were subjected to conditions outlined in example 8 to produce 10 mg of yellow powder in 6% yield. [M H]⁺: 410.0, 412.0, 414.0; ¹H (CDCl₃) δ 8.66 (d, J=4.4 Hz, 1H), 8.13 (d, J=2.0 Hz, 1H), 7.91 (d, J=2.4 Hz, 1H), 7.71 (d, J=7.6 Hz, 1H), 7.40 (dd, J=7.6, 4.4 Hz, 1H), 2.64 (s, 3H).

Compound-300: 2-Isopropyl-6-[3-(3-methyl-pyridin-2-yl)-[1,2,4]oxadiazol-5-yl]-phenol

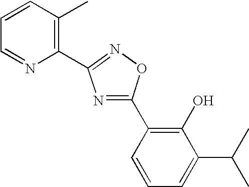

3-isopropyl-2 hydroxybenzoic acid and [O21]N-Hydroxy-3-methyl-pyridine-2-carboxamidine were subjected to conditions outlined in example 8 to produce 9 mg of yellow powder in 5% yield. [M H]⁺: 296.3; ¹H NMR (CDCl₃) δ 8.46 (d, J=4.8 Hz, 1H), 7.78 (dd, J=8.0, 2.0, 1H), 7.65 (td, J=7.6, 0.8 Hz, 1H), 7.44 (dd, J=7.6, 2.0 Hz, 1H), 7.31 (dd, J=7.6, 4.4 Hz, 1H), 6.89 (t, J=7.6 Hz, 1H), 3.40 (spt, J=6.8 Hz, 1H), 1.27 (d, J=6.8 Hz, 6H).

Compound 243: 5-Ethoxy-2-[3-(3-methyl-pyridin-2-yl)-[1,2,4]oxadiazol-5-yl]-phenol

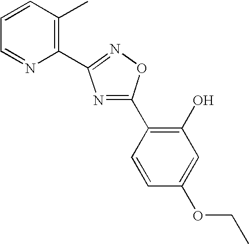

4-Ethoxysalicylic acid and [O21]N-Hydroxy-3-methyl-pyridine-2-carboxamidine were subjected to conditions outlined in example 8 to produce 17 mg of yellow-brown powder in 9% yield. [M H]⁺: 298.1; ¹H NMR (CDCl₃) δ 10.56 (s, 1H), 8.65 (dd, J=4.8, 1.2 Hz, 1H), 7.90 (d, J=9.2 Hz, 1H), 7.69 (d, J=8.0 Hz, 1H), 7.36 (dd, J=7.6, 4.8 Hz, 1H), 6.60 (m, 2H), 4.10 (q, J=7.2 Hz, 2H), 2.66 (s, 3H), 1.46 (t, J=7.2 Hz, 3H).

### EXAMPLE 16

Compound 268: 2-Ethoxy-6-[3-(3-methyl-pyridin-2-yl)-[1,2,4]oxadiazol-5-yl]-phenol

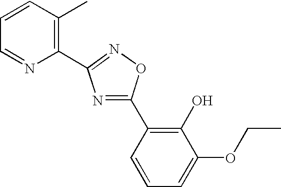

Step 1: Preparation of 3-ethoxysalicylic acid (O24)

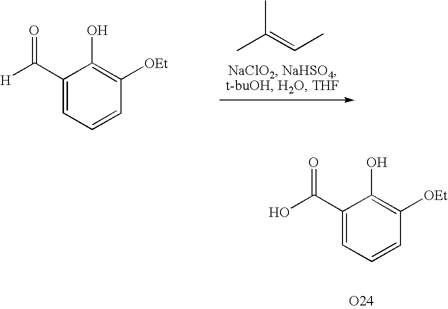

Oxidation of 3-ethoxysalicylaldehyde was carried out using protocol outlined in example 10 to yield 200 mg of desired product [O24]in 94% yield. ¹H NMR (d₆-DMSO) δ 7.20 (dd, J=8.0, 1.6 Hz, 1H), 7.16 (dd, J=8.0, 1.6 Hz), 7.099 (t, J=8.0 Hz, 1H), 4.08 (q, J=6.8 Hz, 1H), 1.36 (t, J=6.8 Hz, 1H)

Step 2

Compound 268: 2-Ethoxy-6-[3-(3-methyl-pyridin-2-yl)-[1,2,4]oxadiazol-5-yl]-phenol

[O24] 2-ethoxysalyclic acid and [O21]N-Hydroxy-3-methyl-pyridine-2-carboxamidine were subjected to protocol outlined in example 8 to produce 2.8 mg of a white solid in 24% yield. [M H]⁺: 298.2; ¹H NMR (CDCl₃) δ 10.54 (s, 1H), 8.66 (d, J=4.4 Hz, 1H), 7.70 (d, J=8.8 Hz, 1H), 7.65 (dd, J=8.0, 1.6 Hz, 1H), 7.38 (dd, J=8.4, 4.8 Hz, 1H), 7.12 (d, J=7.6 Hz, 1H), 6.98 (t, J=8.0 Hz, 1H), 4.18 (q, J=7.2 Hz, 1H), 1.23 (t, J=7.2 Hz, 1H)

### EXAMPLE 17

Compound 267: 4-Benzyloxy-3-[3-(3-methyl-pyridin-2-yl)-[1,2,4]oxadiazol-5-yl]-phenol

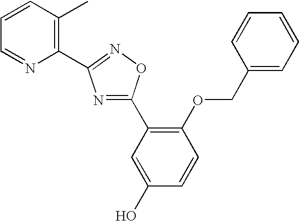

Step 1: Preparation of 2-Benzyloxy-5-hydroxy-benzaldehyde (O25)

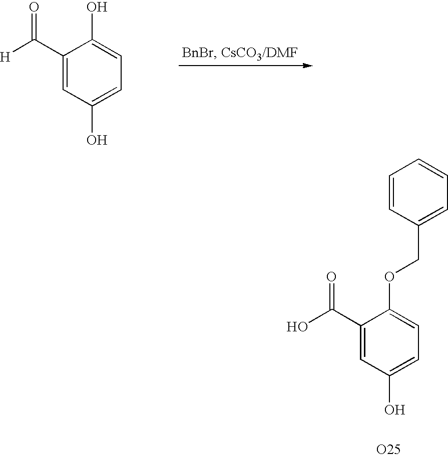

2,5-dihydroxybenzaldehyde (2.14 g, 15.5 mmoles) was dissolved in dry N,N-dimethylformamide (60 ml) under argon. Cesium Carbonate (2.0 equiv, 10.1 g, 31 mmoles) and Benzyl Bromide (1.5 equiv, 2.7 ml, 23.2 mmoles) wete then added. The mixture stirred at ambient temperature for 15 hours and was then concentrated in vacuo. The residue was then partitioned in ethyl acetate and 0.1 N HCL. The organic layer was dried with magnesium sulfate, filtered, and purified by chromatography to produce 1.2 g of a red-brown solid in 34% yield. R_{f}: 0.43 (9:1 CHCl₃/THF); ¹H NMR (CDCl₃) δ 10.35 (s, 1H), 7.918 (s, 1H), 7.28 (m, 6H), 7.00 (dd, J=9.6, 3.2 Hz, 1H), 6.85 (d, J=9.2 Hz), 5.03 (s, 2H).

Step 2: Preparation of [O261 2-Benzyloxy-5-hydroxy-benzoic acid

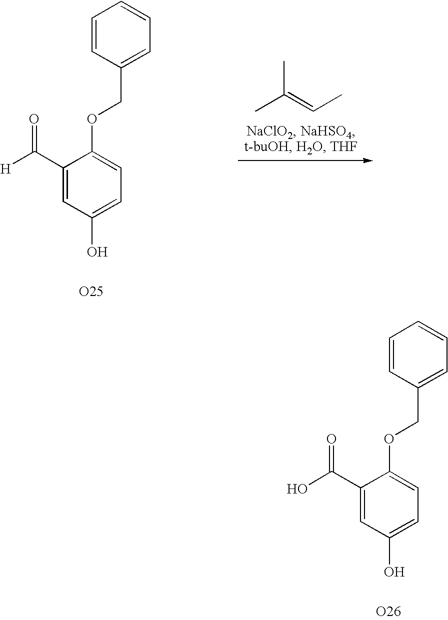

Synthesis was carried out using 2-Benzyloxy-5-hydroxy-benzaldehyde [O25]and protocol outlined in example 10 to yield 433 mg of white powder in 81% yield. R_{f}: 0.17 (9:1 CHCl₃/THF); [M H]⁺: 245.2; ¹H NMR (CDCl₃) δ 7.77 (d, J=3.2 Hz, 1H), 7.40 (m, 5H), 7.09 (dd, J=8.8, 3.2 Hz, 1H), 7.00 (d, J=8.8 Hz, 1H), 5.21 (s, 2H).

Step 3

Compound 267: 4-Benzyloxy-3-[3-(3-methyl-pyridin-2-yl)-[1,2,4]oxadiazol-5-yl]-phenol

Synthesis was carried out with 2-Benzyloxy-5-hydroxy-benzoic acid [O26]and N-Hydroxy-3-methyl-pyridine-2-carboxamidine[O21]were subjected to protocol outlined in example 8 to produce 103 mg of white powder in 14% yield. [M H]⁺: 360.3; ¹H NMR (CDCl₃) δ 8.61 (s, 1H), 8.57 (d, J=3.6 Hz, 1H), 7.64 (d, J=2.8 Hz., 1H), 7.60 (d, J=8.0, 1H), 7.47 (d, J=7.6 Hz, 1H), 7.26 (m, 5H), 6.97 (dd, J=8.8, 2.8 Hz, 1H), 6.91 (d, J=8.8 Hz, 1H), 5.12 (s, 2H), 2.58 (s, 3H)

### EXAMPLE 18

Compound 271: 4-Ethoxy-2-[3-(3-methyl-pyridin-2-yl)-[1,2,4]oxadiazol-5-yl]-phenol

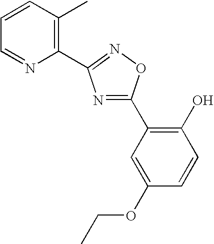

Step 1: Preparation of 2-{5-[5-Ethoxy-2-(2-methylene-pent-3-enyloxy)-phenyl]-[1,2,4]oxadiazol-3-yl}-3-methyl-pyndine (O27)

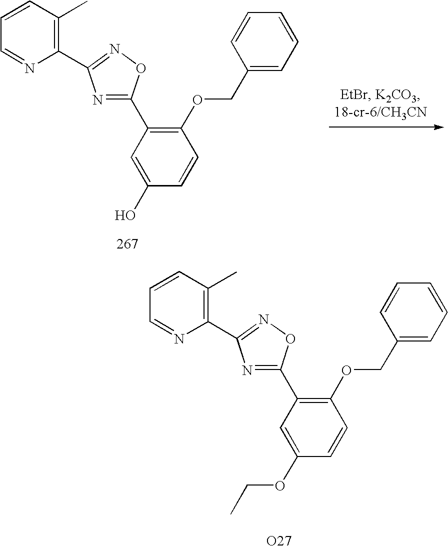

4-Benzyloxy-3-[3-(3-methyl-pyridin-2-yl)-[1,2,4]oxadiazol-5-yl]-phenol (267) (33 mg, 0.09 mmoles) was dissolved in acetonitrile (0.2 ml). Potassium carbonate (1.5 equiv, 18 mg, 0.13 mmoles) and ethyl bromide (1.5 equiv, 10 μl, 0.13 mmoles), and 18-crown-6 (1.5 equiv, 34 mg, 0.13 mmoles) were then added. The mixture stirred at ambient temperature for 6 h. The crude material was then purified by chromatography to produce 23 mg of a clear oily residue in 66% yield. R_{f}: 0.52 (20:1 CH₂Cl₂/MeOH); ¹H NMR (CDCl₃) δ 8.68 (dt, J=4.8, 0.8 Hz, 1H), 7.77 (d, J=2.8 Hz, 1H), 7.67 (d, J=7.6 Hz, 1H), 7.54, (d, J=7.2 Hz, 1H), 7.34 (m, 5H), 7.05 (m, 2H), 5.28 (s, 2H), 4.06 (q, J=6.8 Hz, 2H), 2.68 (s, 3H), 1.41 (t, J=6.8 Hz, 3H)

Step 2

Compound 271: 4-Ethoxy-2-[3-(3-methyl-pyridin-2-yl)-[1,2,4]oxadiazol-5-yl]-phenol

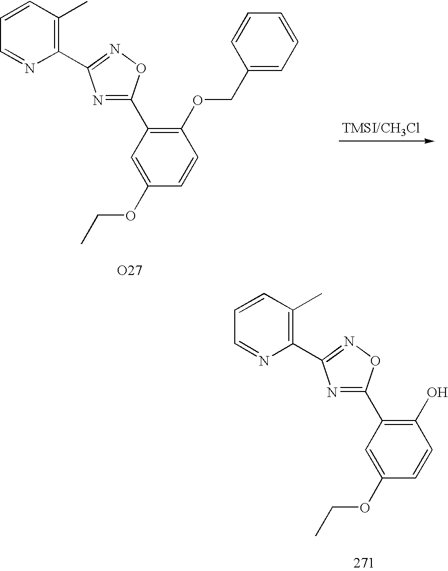

2-[5-(2-Benzyloxy-5-ethoxy-phenyl)-[ 1 ,2,4]oxadiazol-3-yl]-3-methyl-pyridine (O27) (23 mg, 0.06 mmoles) was dissolved in chloroform (300 μl). Iodotrimethylsilane (3.0 equiv, 29 μl, 0.18 mmoles) was then added and the reaction stirred for 2 h at 50° C. The reaction was quenched with the addition of methanol (1 ml). The mixture was then dried in vacuo, and purified by chromatography to produce 18 mg of a yellow powder in quantitative yield. R_{f}: 0.25 (20:1 CH₂Cl₂/MeOH); ¹H NMR (CDCl₃) δ 10.04 (s, 1H), 8.65 (dd, J=4.8, 1.2 Hz, 1H), 7.70 (dq, J=8.0, 0.8 Hz), 7.45 (d, J=2.8 Hz, 1H), 7.37 (dd, J=7.6, 4.4 Hz, 1H), 7.13 (dd, J=8.8, 2.8 Hz, 1H), 7.06 (d, J=8.8 Hz, 1H), 4.07 (q, J=7.2 Hz, 2H), 2.66 (s, 3H), 1.45 (t, 7.2 Hz, 3H)

Compound 272: 2-[3-(3-Methyl-pyridin-2-yl)-[1,2,4]oxadiazol-5-yl]-4-propoxy-phenol

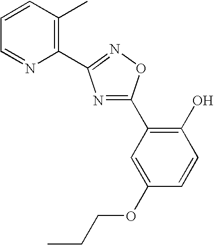

4-Benzyloxy-3-[3-(3-methyl-pyridin-2-yl)-[1,2,4]oxadiazol-5-yl]-phenol [267]was alkylated with propylbromide, and then debenzylated using protocol as outlined in example 18 to produce 7.5 mg of a yellow solid in 24% yield. R_{f}: 0.22 (20:1 CH₂Cl₂/MeOH); ¹H NMR (CDCl₃) δ 10.04 (s, 1H), 8.65 (dd, J=4.8, 1.6 Hz, 1H), 7.70 (dd, J=8.8, 0.8 Hz, 1H), 7.46 (d, J=3.2 Hz, 1H), 7.37 (dd, J=7.6, 4.4 Hz), 7.13 (dd, , J=9.2, 2.8 Hz, 1H), 3.96 (t, J=6.8 Hz, 2H), 2.66 (s, 3H), 1.84 (sx, J=7.2 Hz, 2H), 1.08 (t, J=7.2 Hz, 3H)

Compound 273: 2-[3-(3-Methyl-pyridin-2-yl)-[1,2,4]oxadiazol-5-yl]-4-phenethyloxy-phenol

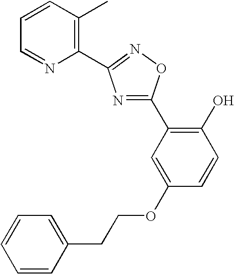

4-Benzyloxy-3-[3-(3-methyl-pyridin-2-yl)-[1,2,4]oxadiazol-5-yl]-phenol [267]was alkylated with 2-(bromoethyl)benzene and then debenzylated using protocol outlined in example 18 to produce 12 mg of yellow powder in 38% yield. R_{f}: 0.12 (20:1 CH₂Cl₂/MeOH); [M H]⁺: 374.2; ¹H NMR (CDCl₃) δ 10.04 (s, 1H), 8.65 (d, J=4.4, 1H), 7.69 (dd, J=7.6, 0.8 Hz, 1H), 7.45 (d, J=2.8 Hz, 1H), 7.37 (dd, J=7.6, 4.8, 1H), 7.31 (m,5H), 7.19 (dd, J=8.8, 2.8 Hz, 1H), 4.22 (t, J=6.8 Hz, 2H), 3.70 (s, 3H), 3.13 (t, 2H).

### EXAMPLE 19

Compound 298: {2-Hydroxy-3-[3-(3-methyl-pyridin-2-yl)-[1,2,4]oxadiazol-5-yl]-phenyl}-carbamic acid tert-butyl ester

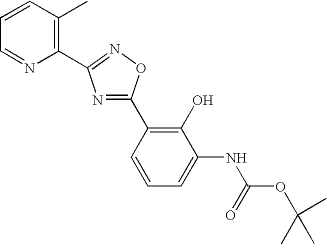

Step 1: Preparation of 3-tert-Butoxycarbonylamino-2-hydroxy-benzoic acid (O28)

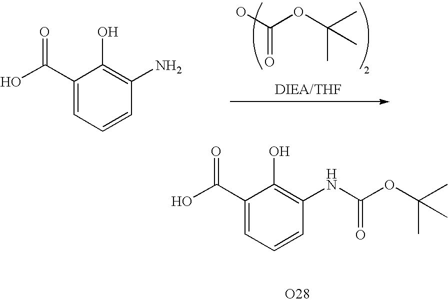

3-aminosalicylic acid (1.0 g, 6.5 mmoles) was dissolved in dry tetrahydrofuran (50 ml) under argon. Diisopropylethylamine (2.0 equiv, 2.3 ml, 13.0 mmoles) and di-t-butyldicarbonate (1.5 equiv, 2.1 g, 9.7 mmoles) were added. The reaction stirred at ambient temperature for 18 h. The mixture was then concentrated in vacuo, diluted in ethyl acetate and partitioned with 0.1 N aqueous HCl. The organic layer was dried over magnesium sulfate, filtered, and purified by chromatography. 770 mg of a grey-white solid was isolated in 47% yield. R_{f}: 0.26 (20:1 CH₂Cl₂/MeOH); [M]⁻: 252.3; ¹H NMR (d₆-DMSO) δ 8.02 (s, 1H), 7.82 (d, J=8.0 Hz, 1H), 7.48 (dd, J=8.0, 1.6 Hz, 1H), 6.85 (t, J=8.0, 1H), 1.45 (s, 9H)

Step 2

Compound 298: {2-Hydroxy-3-[3-(3-methyl-pyridin-2-yl)-[1,2,4]oxadiazol-5-yl]-phenyl}-carbamic acid tert-butyl ester

Synthesis was carried out with 3-tert-Butoxycarbonylamino-2-hydroxy-benzoic acid [O28]and N-Hydroxy-3-methyl-pyridine-2-carboxamidine [O21]using protocol outlined in example 8 to produce 123 mg of white powder in 40% yield. [M H]⁺: 369.3; ¹H NMR (CDCl₃) δ 8.67(dt, J=4.4, 0.8 Hz, 1H), 8.34 (d, J=7.6 Hz, 1H), 7.70 (dt, J=6.8, 0.8 Hz, 1H), 7.67 (dd, J=8.0, 1.2 Hz, 1H), 7.39 (dd, J=7.6, 4.8 Hz, 1H), 7.24 (b, 1H), 7.038 (t, J=8.0 Hz, 1H), 2.65 (s, 3H), 1.57 (s, 9H).

Compound 276: {3-Hydroxy-4-[3-(3-methyl-pyridin-2-yl)-[1,2,4]oxadiazol-5-yl]-phenyl}-carbamic acid tert-butyl ester

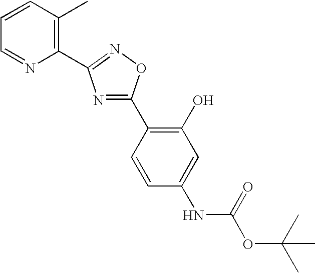

The synthesis was carried out with BOC protected 4-aminosalicylic acid and and N-Hydroxy-3-methyl-pyridine-2-carboxamidine using protocol outlined in example 19 to produce 170 mg of white powder in 25% yield. R_{f}: 0.24 (9:1 CHCl₃/THF); [M H]⁺: 369.3; ¹H NMR (CDCl₃) δ 10.44 (s, 1H), 8.63 (s, 1H), 7.88 (d, J=8.8 Hz, 1H), 7.68 (d, J=7.2 Hz, 1H), 7.35 (s, 1H), 7.21 (s, 1H), 7.04 (d, J=8.4 Hz, 1H), 6.87 (s, 1H), 2.64 (s, 3H), 1.52 (s, 9H).

### EXAMPLE 20

Compound 277: 5-Amino-2-[3-(3-methyl-pyridin-2-yl)-[1,2,4]oxadiazol-5-yl]-phenol

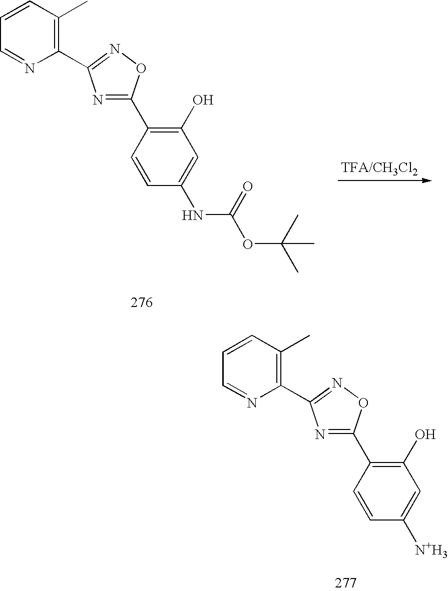

{3-Hydroxy-4-[3-(3-methyl-pyridin-2-yl)-[1,2,4]oxadiazol-5-yl]-phenyl}-carbamic acid tert-butyl ester (276) (134 mg, 0.36 mmoles) was dissolved in dichloromethane (500 μl). trifluoroacetic acid (500 μl ) was then added. The mixture stirred for 30 m at ambient temperature, and was then diluted in dichloromethane, and partitioned with aqueous 5% sodium bicarbonate. The organic layer was dried over magnesium sulfate, filtered, and dried in vacuo to produce 125 mg the TFA salt as a white powder in quantitative yield. R_{f}: 0.40 (9:1 CH₂Cl₂/MeOH); [M H]⁺: 269.2; ¹H NMR (d₆-DMSO) δ 10.30 (s,1H), 8.58 (d, J=2.4 Hz, 1H), 7.64 (d, J=8.8 Hz, 1H), 7.49 (dd, J=7.6, 4.8 Hz, 1H), 6.26 (dd, J=8.8, 2.0Hz, 1H), 6.17 (s, 3H), 2.48 (s, 3H).

### EXAMPLE 21

Compound 291: N-{3-Hydroxy-4-[3-(3-methyl-pyridin-2-yl)-[1,2,4]oxadiazol-5-yl]-phenyl}-acetamide

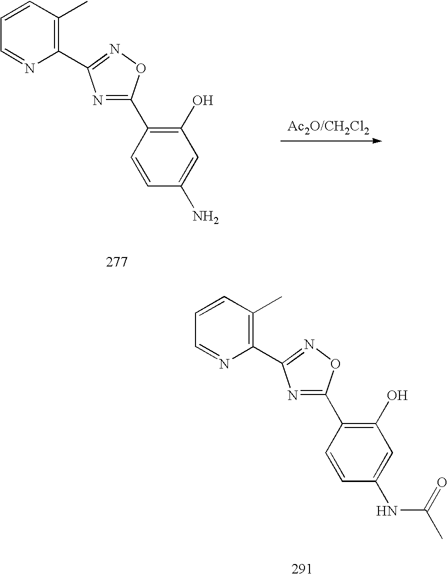

5-Amino-2-[3-(3-methyl-pyridin-2-yl)-[1,2,4]oxadiazol-5-yl]-phenol (277) (25 mg, 0.09 mmoles) was dissolved in dry N,N-dimethylformamide (250 μl). Acetic anhydride (1.0 equiv, 9 μl, 0.09 mumoles) and diisopropylethylamine (2.0 equiv, 31 ,l, 0.18 mmoles) were then added, and the reaction stirred for 15 hours at ambient temperature. The crude mixture was dried in vacuo, and purified by chromatography to produce 8 mg of white powder in 28% yield. [M H]⁺: 311.3; ¹H NMR (CDCl₃) δ 8.64 (d, J=4.4 Hz, 1H), 8.13 (d, J=8.8 Hz, 1H). 7.66 (d, J=7.6 Hz, 1H), 7.32 (dd, J=7.6, 4.8 Hz, 1H), 6.62 (dd, J=8.8, 2.0 Hz, 1H), 6.44 (d, J=2 Hz, 1H), 2.68 (s, 3H), 2.48 (s, 3H).

### EXAMPLE 22

Compound 289: N-{3-Hydroxy-4-[3-(3-methyl-pyridin-2-yl)-[1,2,4]oxadiazol-5-yl]-phenyl}-benzamide

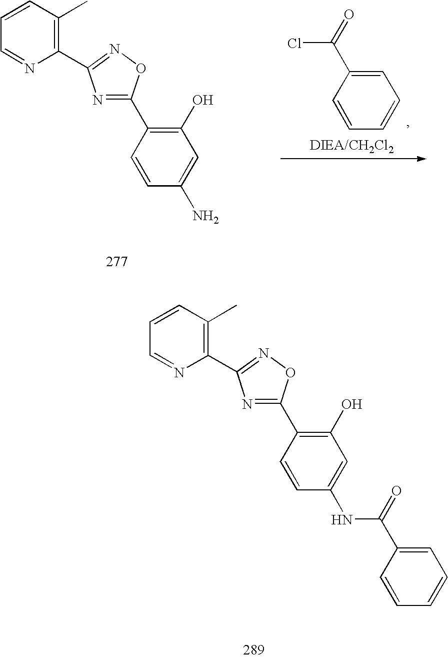

5-Amino-2-[3-(3-methyl-pyridin-2-yl)-[1,2,4]oxadiazol-5-yl]-phenol (277) (25 mg, 0.09 mmoles) was dissolved in CH₂Cl₂ (250 μl) and cooled to 0° C. Benzoyl chloride (1.0 equiv, 10 μl, 0.09 mmoles) and diisopropylethylamine (2 equiv, 31 μl, 0.18 mmoles) were added, and stirred for 1 h with warming to ambient temperature. The crude mixture was then dried in vacuo, and purified by chromatography to produce 5 mg of white powder in 12% yield. [M H]⁺: 373.3; ¹H NMR (CDCl₃) δ 8.72 (d, J=4.0, 1H), 8.28 (dd, J=8.0, 1.2 Hz, 1H), 8.20 (d, J=8.8 Hz, 1H), 7.75 (d, J=8.0, 1H), 7.65 (t, J=7.2 Hz, 1H), 7.52 (t, J=8.0 Hz, 2H), 7.44, (dd, J=7.6, 5.2 Hz, 1H), 6.69 (dd, J=8.8, 2.0 Hz, 1H), 6.60 (d, J=2.0 Hz, 1H), 2.50 (s, 1H).

### EXAMPLE 23

Compound 288: 5-Benzylamino-2-[3-(3-methyl-pyridin-2-yl)-[1,2,4]oxadiazol-5-yl]-phenol

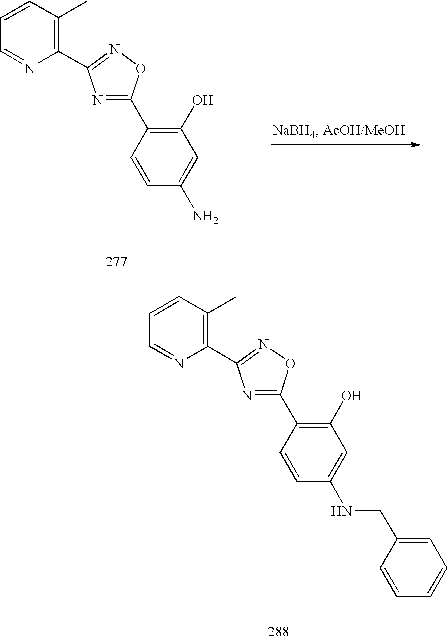

5-Amino-2-[3-(3-methyl-pyridin-2-yl)-[1,2,4]oxadiazol-5-yl]-phenol (277) (25 mg, 0.09 mmoles) and benzaldehyde (5 equiv, 46 μl, 0.45 mmoles) were dissolved in methanol (2 ml) and glacial acetic acid (100 μl). Sodium borohydride (1.0 equiv, 4 mg, 0.09 mmoles) was then added with stirring at ambient temperature for lh. The mixture was concentrated in vacuo and purified by chromatography to prduce 8 mg of yellow powder in 25% yield. [M H]⁺: 359.3 ; ¹H NMR (d₆-DMSO) δ 10.35 (s, 1H), 8.58 (s, 1H), 7.83 (m, 1H), 7.66 (d, J=8.8 Hz, 1H), 7.48 (m, 1H), 7.32 (m,4H), 7.23 (m, 1H), 6.37 (d, J=8.4 Hz, 1H), 6.15 (s, 1H), 5.72 (s, 1H), 5.69 (s, 1H), 4.34 (s, 2H), 2.52 (s, 3H)

Compound 266: {4-Hydroxy-3-[3-(3-methyl-pyridin-2-yl)-[1,2,4]oxadiazol-5-yl]-phenyl}-carbamic acid tert-butyl ester

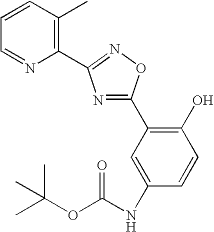

The synthesis was carried out with BOC protected 4-aminosalicylic acid and and [O21]N-Hydroxy-3-methyl-pyridine-2-carboxamidine using protocol outlined in example 19 to produce 305 mg of clear crystals in 31% yield. [M H]⁺: 369.4; 1H NMR (CDCl₃) δ 8.65 (dd, J=4.0, 0.8 Hz, 1H), 8.04 (d, J=2.4 Hz, 1H), 7.69 (dq, J=7.6, 0.8 Hz, 1H), 7.46 (d, J=7.2 Hz, 1H), 7.37 (dd, J=7.6, 4.8 Hz, 1H), 7.06 (d, J=8.8 Hz, 1H), 6.57 (b, 1H), 2.63 (s, 3H), 1.55 (s, 9H).

Compound 270: 4-Amino-2-[3-(3-methyl-pyridin-2-yl)-[1,2,4]oxadiazol-5-yl]-phenol

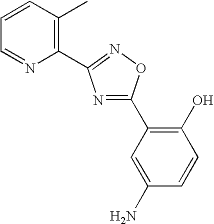

Deprotection of {4-Hydroxy-3-[3-(3-methyl-pyridin-2-yl)-[1,2,4]oxadiazol-5-yl]-phenyl}-carbamic acid tert-butyl ester was carried out using the protocol outlined in example 20 to produce 535 mg of white powder as the TFA salt in quantitative yield. [M H]⁺: 269.3; ¹H NMR (CDCl₃) δ 8.50 (dd, J=4.8, 1.2 Hz, 1H), 7.67 (d, J=2.4 Hz, 1H), 7.60 (dd, J=8.0, 0.8 Hz, 1H), 7.25 (m, 2H), 6.94 (d, J=8.8 Hz, 1H), 2.52 (s, 3H).

Compound 275: N-{4-Hydroxy-3-[3-(3-methyl-pyridin-2-yl)-[1,2,4]oxadiazol-5-yl]-phenyl}-acetamide

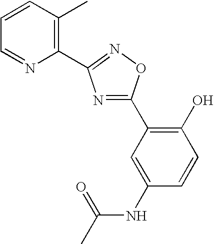

The synthesis was carried out using 4-Amino-2-[3-(3-methyl-pyridin-2-yl)-[1,2,4]oxadiazol-5-yl]-phenol and the protocol outlined in example 21 to produce 21 mg of a yellow oily solid in 84% yield. [M H]⁺: 311.3; ¹H NMR (CDCl₃) δ 8.64 (b, 1H), 8.06 (b, 1H), 7.68 (d, J=7.2 Hz, 1H), 7.53 (d, J=10.0 Hz, 1H), 7.35 (m, 1H), 7.11 (s, 1H), 7.02 (d, J=8.8 Hz, 1H), 2.608 (s, 3H), 2.128 (s, 1H).

Compound 290: N-{4-Hydroxy-3-[3-(3-methyl-pyridin-2-yl)-[1,2,4]oxadiazol-5-yl]-phenyl}-benzamide

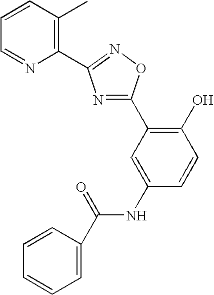

The synthesis was carried out using 4-Amino-2-[3-(3-methyl-pyridin-2-yl)-[1,2,4]oxadiazol-5-yl]-phenol and the protocol outlined in example 22 to produce 4.5 mg of white powder in 12% yield. [M H]⁺: 373.3; ¹H NMR (CDCl₃) δ 8.76 (d, J=3.2 Hz, 1H), 8.33 (d, J=2.8Hz, 1H), 7.90 (t, J=6.8 Hz, 2H), 7.79 (t, J=8.0, 2H), 7.51 (m, 3H), 7.25 (s, 1H), 7.18 (d, J=9.2 Hz, 1H), 2.70 (s, 3H).

### EXAMPLE 24

Compound 297: 4-(5,5-Dimethyl-[1,3]dioxan-2-yl)-2-[3-(3-methyl-pyridin-2-yl)-[1,2,4]oxadiazol-5-yl]-phenol

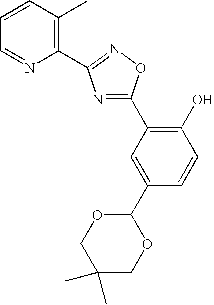

Step 1: Preparation of 5-(5,5-Dimethyl-rl3]dioxan-2-yl)-2-hvdroxy-benzoic acid (O29)

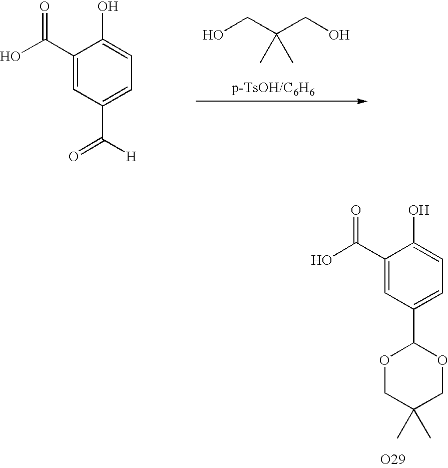

5-formylsalicylic acid (530 mg, 3.2 mmoles) and 2,2-dimethyl-1,3-propanediol (1.1 equiv, 366 mg, 3.5 mmoles) were dissolved in dry benzene (12 ml). P-toluenesulfonic acid (0.01 equiv, 6 mg, 0.03 mmoles) was then added, and the mixture was heated to reflux for 15 h in a flask fitted with a Dean-Stark trap and condenser. The mixture was then concentrated in vacuo and purified by chromatography to produce 782 mg of a white solid in 97% yield. [M]⁻: 251.4; ¹H NMR (d₆-DMSO) δ 7.82 (d, J=2.4 Hz, 1H), 7.52 (dd, J=8.4, 2.0 Hz, 1H), 6.919 (d, J=8.8 Hz, 1H), 5.36 (s, 1H), 3.62 (q, J=10.4 Hz, 4 H), 1.16 (s, 3H), 0.74 (s, 3H).

Step 2

Compound 297: 4-(5,5-Dimethyl-[1,3]dioxan-2-yl)-2-[3-(3-methyl-pyridin-2-yl)-[1,2,4]oxadiazol-5-yl]-phenol

The synthesis was carried out with 5-(5,5-Dimethyl-[1,3]dioxan-2-yl)-2-hydroxy-benzoic acid (O29) and N-Hydroxy-3-methyl-pyridine-2-carboxamidine (O21) using protocol outlined in example 8 to produce 96 mg of a yellow solid in 48% yield. [M H]⁺: 368.4; ¹H NMR (CDCl₃) δ 8.66 (dd, J=3.6, 1.6 Hz, 1H), 8.19 (d, J=2.4 Hz, 1H), 7.69 (m, 2H), 7.38 (dd, J=7.6, 4.4 Hz, 1H), 7.15 (d, J=8.8Hz, 1H), 5.42 (s, 1H), 3.81 (d, J=11.2 Hz, 2H), 3.69 (d, J=11.2 Hz, 2H), 2.67 (s, 3H), 1.33 (s, 3H), 0.84 (s, 3H).

Compound 246: 2-Methoxy-6-[3-(3-methoxy-pyridin-2-yl)-[1,2,4]oxadiazol-5-yl]-phenol

### EXAMPLE 25

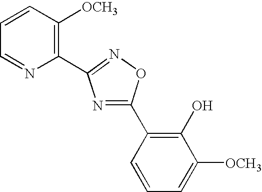

Step 1: Preparation of 3-Methoxy-nyridine 1-oxide (O30)

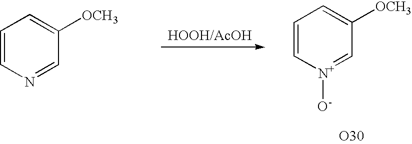

3-methoxy pyridine (10.0 g, 91.6 mmoles) was subjected to conditions outlined in Example 8 to yield 11.45 g of white powder in quantitative yield. ¹H NMR (CDCl₃) δ 8.20 (s, 1H), 8.00 (d, J=6.4 Hz, 1H), 7.22 (dd, J=8.4, 6.0 Hz), 6.99 (dd, J=8.8, 2.0 Hz, 1H), 3.85 (s, 3H).

Step 2: Preparation of 3-Methoxy-pyridine-2-carbonitrile (O31)

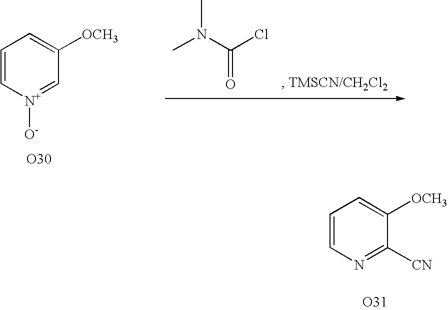

3-Methoxy-pyridine 1-oxide (O30) (11.45 g, 91.6 mmoles) was subjected to conditions outlined in Example 8 to yield 9.0 g of white powder with selective substitution at the ortho position in 73% yield. [M H]⁺: 135.0; ¹H NMR (CDCl₃) δ 8.28 (dd, J=4.4, 1.2 Hz, 1H), 7.47 (dd, J=8.4, 4.4 Hz, 1H), 7.34 (dd, J=8.4, 1.2 Hz, 1H), 3.68 (s, 3H).

Step 3: Preiparation of N-Hydroxy-3-methoxy-pyridine-2-carboxamidine (O32)

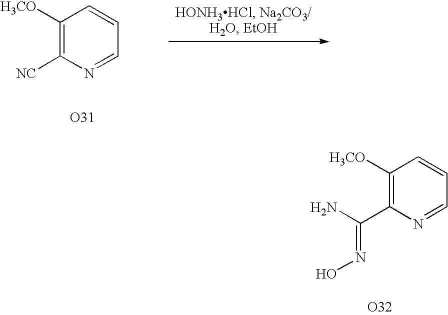

3-Methoxy-pyridine-2-carbonitrile (O31) (5.0 g, 37 mmoles) was reacted in the conditions detailed in example 8 to yield 3.4 g of white-yellow crystals in 55% yield. [M H]⁺: 168.0; 1H NMR (CDCl₃) δ 9.66 (s, 1H), 8.13 (dd, J=4.4, 1.2 Hz, 1H), 7.49 (d, J=8.4 Hz, 1H), 7.37 (dd, J=8.4, 4.4 Hz, 1H), 5.60 (s, 2H), 3.78 (s, 3H).

Step 2

Compound 246: 2-Methoxy-6-13-(3-methoxy-pyridin-2-yl)-[1,2,4]oxadiazol-5-yl]-phenol

N-Hydroxy-3-methoxy-pyridine-2-carboxamidine (O32) and 3-methoxysalicylic acid were subjected to protocol outlined in Example 8 to produce 45 mg of beige powder in 30% yield. [M H]⁺: 300.3.

Compound 247: 5-Methoxy-2-[3-(3-methoxy-pyridin-2-yl)-[1,2,4]oxadiazol-5-yl]-phenol

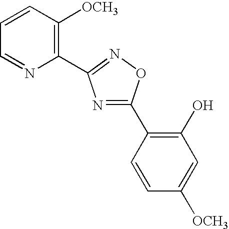

The synthesis was carried out with N-Hydroxy-3-methoxy-pyridine-2-carboxamidine (O32) and 4-methoxysalicylic acid using protocol detailed in Example 8 to produce 26 mg of white powder in 19% yield. [M H]⁺: 300.1.

Compound 248: 4-Methoxy-2-[3-(3-methoxy-pyridin-2-yl)-[1,2,4]oxadiazol-5-yl]-phenol

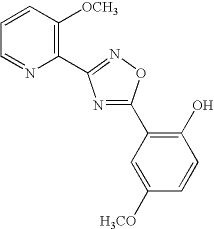

The synthesis was carried out with N-Hydroxy-3-methoxy-pyridine-2-carboxamidine (O32) and 5-methoxysalicylic acid using protocol detailed in Example 8 to produce 7.3 mg of white powder in 17% yield. [M H]⁺: 300.2.

Compound 249: 4-Bromo-2-[3-(3-methoxy-pyridin-2-yl)-[1,2,4]oxadiazol-5-yl]-phenol

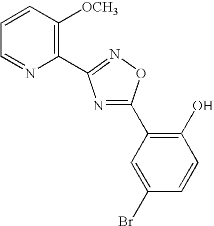

The synthesis was carried out with N-Hydroxy-3-methoxy-pyridine-2-carboxamidine (O32) and 5-bromosalicylic acid using protocol detailed in Example 8 to produce 31 mg of white powder. [M H]⁺: 350.0 ¹H NMR (CDCl₃) δ 10.66 (s, 1H), 8.43 (dd, J=4.4, 1.2 Hz, 1H), 8.13 (d, J=2.4 Hz, 1H), 7.59 (dd, J=8.8, 2.8 Hz, 1H), 7.47 (m, 2H), 7.05 (d, J=9.2 Hz, 1H), 4.01 (s, 3H).

Compound 250: 2-[3-(3-Methoxy-pyridin-2-yl)-[1,2,4]oxadiazol-5-yl]-4-methyl-phenol

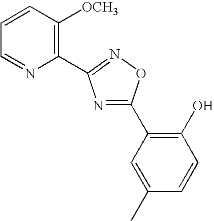

The synthesis was carried out with N-Hydroxy-3-methoxy-pyridine-2-carboxamidine (O32) and 5-bromosalicylic acid using protocol detailed in Example 8 to produce 31 mg of white powder. [M H]⁺: 284.2; ¹H NMR (CDCl₃) δ 10.43 (s, 1H), 8.42 (dd, J=4.0, 1.6 Hz, 1H), 7.81 (s, 1H), 7.45 (m, 2H), 7.31 (dd, J=8.4, 2.8 Hz, 1H), 7.03 (d, J=8.4 Hz, 1H), 4.00 (s, 3H), 2.38 (s, 3H).

Compound 256: 1-[3-(3-Bromo-pyridin-2-yl)-[1,2,4]oxadiazol-5-yl]-naphthalen-2-ol

### EXAMPLE 26

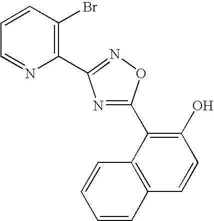

Step 1: Preparation of 3-Bromo-N-hydroxy-pyridine-2-carboxamidine (O33)

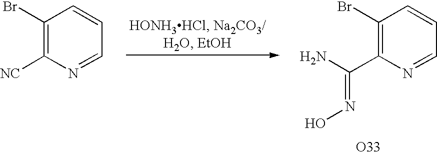

3-bromo-2-cyanopyridine (500 mg, 2.7 mmoles) was subjected to reaction conditions detailed in the protocol of Example 1 to produce 402 mg of yellow powder in 69% yield. ¹H NMR (CDCl₃) δ 8.55 (dd, J=4.4, 1.2 Hz, 1H), 8.12 (br s, 1H), 8.00 (dd, J=8.0, 1.6 Hz), 7.18 (dd, J=8.0, 4.4 Hz, 1H), 5.48 (br s, 2H).

Step 2

Compound 256: 1-[3-(3-Bromo-pyridin-2-yl)-[1,2,4]oxadiazol-5-yl]-naphthalen-2-ol

The synthesis was carried out with 3-Bromo-N-hydroxy-pyridine-2-carboxamidine (O33) and 2-hydroxy-1-naphthoic acid using the protocol outlined in example 8 to produce 4 mg of brown oily solid in 4% yield. [M H]⁺: 368.2, 370.2; ¹H NMR (CDCl₃) δ 8.90 (d, J=0.8 Hz, 1H), 8.69 (dd, J=4.8, 1.6 Hz, 1H), 8.04 (dd, J=8.0, 2.8 Hz, 1H), 7.90 (d, J=9.2 Hz, 1H), 7.76 (d, J=8.0 Hz, 1H), 7.63 (td, J=8.4, 1.6 Hz, 1H), 7.38 (td, J=8.0, 1.2 Hz, 1H), 7.31 (dd, J=7.6, 3.2 Hz, 1H), 7.24 (d, J=8.8 Hz, 1H).

Compound 257: 2-[3-(3-Bromo-pyridin-2-yl)-[1,2,4]oxadiazol-5-yl]-6-methoxy-phenol

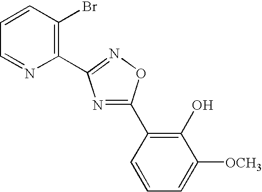

The synthesis was carried out with 3-Bromo-N-hydroxy-pyridine-2-carboxamidine (O33) and 3-methoxysalicylic acid using the protocol outlined in example 8 to produce 6 mg of yellow solid in 8% yield. [M H]⁺: 348.2, 350.2; ¹H NMR (CDCl₃) δ 8.75 (dd, J=4.4, 1.2 Hz, 1H), 8.11 (dd, J=8.0, 1.2 Hz, 1H), 7.64 (dd, J=7.6, 1.6 Hz, 1H), 7.37 (dd, J=8.4, 4.8 Hz, 1H), 7.12 (dd, J=8.0, 1.6 Hz, 1H), 7.01 (t, J=8.4 Hz, 1H), 3.95 (s, 3H).

Compound 258: 2-[3-(3-Bromo-pyridin-2-yl)-[1,2,4]oxadiazol-5-yl]-4-methoxy-phenol

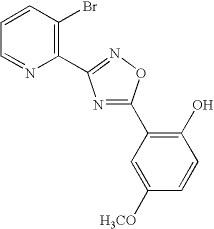

The synthesis was carried out with 3-Bromo-N-hydroxy-pyridine-2-carboxamidine (O33) and 5-methoxysalicylic acid using the protocol outlined in example 8 to produce 5 mg of yellow solid in 6% yield. [M H]⁺: 348.1, 350.1; ¹H NMR (CDCl₃) δ 10.029 (s, 1H), 8.75 (dd, J=4.8, 1.6 Hz, 1H), 8.11 (dd, J=8.0, 1.2 Hz, 1H), 7.46 (d, J=2.8 Hz, 1H), 7.37 (m, 2H), 7.15 (dd, J=8.8, 2.8 Hz, 1H), 7.08 (d, J=8.8 Hz, 1H), 3.87 (s, 3H).

Compound 259: 2-[3-(3-Bromo-pyridin-2-yl)-[1,2,4]oxadiazol-5-yl]-5-methoxy-phenol

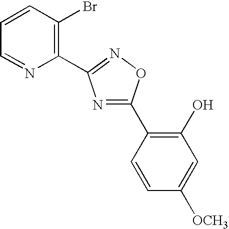

The synthesis was carried out with 3-Bromo-N-hydroxy-pyridine-2-carboxamidine (O33) and 4-methoxysalicylic acid using the protocol outlined in example 8 to produce 3 mg of yellow solid in 3% yield. [M H]⁺: 348.2, 350.2; ¹H NMR (CDCl₃) δ 10.75 (br s, 1H), 8.74 (dd, J=4.8, 1.2 Hz, 1H), 8.10 (dd, J=8.4,.1.6 Hz, 1H), 7.92 (dd, J=7.2, 2.0 Hz, 1H), 7.36 (dd, J=8.0, 4.4 Hz, 1Hz), 6.64 (dd, J=8.0, 2.4 Hz, 1H), 6.62 (s, 1H), 3.90 (s,3H).

Compound 228: 4-Bromo-2-[3-(3-phenyl-pyridin-2-yl)-[1,2,4]oxadiazol-5-yl]-phenol

### EXAMPLE 27

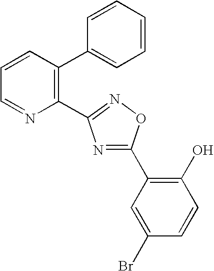

Step 1: Preparation of 3-Phenyl-pyridine-2-carbonitrile (O34)

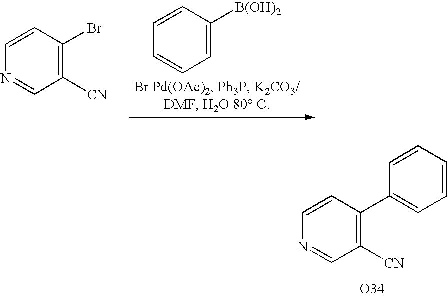

3-Bromo-2-cyanopyridine (4.35 g, 23.8 mmoles) was dissolved in N,N-Dimethylfornamide (40 ml) and water (4 ml). Phenylboronic acid (1.0 equiv, 2.90 g, 23.8 10 mmoles), potassium carbonate (2.0 equiv, 6.56 g, 47.6 mmoles), triphenylphosphine (0.1 equiv, 600 mg, 2.3 mmoles), and palladium acetate (0.1 equiv, 515 mg, 2.3 mmoles) were then added. The mixture was heated to eighty degrees centigrade for 24 hours then cooled to ambient temperature. The residue was partitioned between ethyl acetate and water. The organic layer was purified by chromatography to produce 3.8 g of solid in 89% yield. ¹H NMR (CDCl₃) δ 8.60 15 (dd, J=4.8, 1.6 Hz, 1H), 7.77 (dd, J=8.0, 1.6 Hz, 1H), 7.45 (m, 6H).

Step 2: Preparation of N-Hydroxy-3-phenyl-pyridine-2-carboxamidine

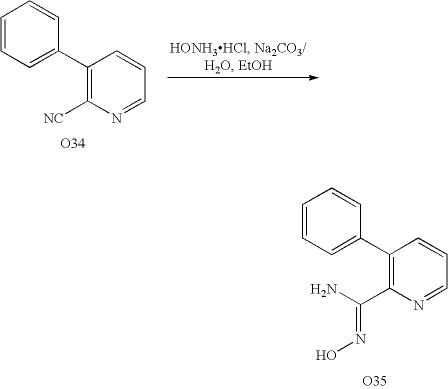

3-Phenyl-pyridine-2-carbonitrile (O34) was subjected to the protocol detailed in example 8 to produce 2.5 g of yellow crystals in quantitative yield. [M H]⁺: 214.1; ¹H NMR (d₆-DMSO) δ 9.33 (s, 1H), 8.55 (d, J=4.8 Hz, 1H), 7.74 (d, J=8.0 Hz, 1H), 7.36 (m, 6H), 5.68 (s, 2H).

Step 3

Compound 228: 4-Bromo-2-[3-(3-phenyl-pyridin-2-yl) -[1,2,4]oxadiazol-5-yl]-phenol

The synthesis was carried out with N-Hydroxy-3-phenyl-pyridine-2-carboxatnidine (O35) and 5-bromosalicylic acid using the protocol detailed in Example 1 to produce 16 mg of beige powder in 20% yield. [M H]⁺: 394.2, 396.2; ¹H NMR (CDCl₃) δ 9.36 (s, 1H), 8.72 (d, J=4.8, 1.2 Hz, 1H), 7.93 (d, J=2.4 Hz, 1H), 7.75 (dd, J=8.0, 1.6 Hz, 1H), 7.46 (dd, J=8.0, 4.8, Hz, 1H), 7.42 (dd, J=8.8, 2.4 Hz, 1H), 7.31 (m, 3H), 7.19 (m, 2H), 6.82 (d, J=9.2 Hz, 1H).

Compound 229: 1-[3-(3-Phenyl-pyridin-2-yl)-[1,2,4]oxadiazol-5-yl]-naphthalen-2-ol

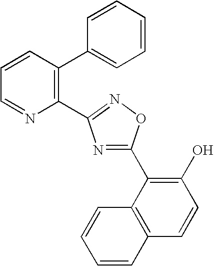

The synthesis was carried out with N-Hydroxy-3-phenyl-pyridine-2-carboxamidine (O35) and 2-hydroxy-1-naphthoic acid using the protocol detailed in Example 8 to produce 14 mg of brown oily solid in 17% yield. [M H]⁺: 366.3; ¹H NMR (CDCl₃) δ 8.88 (d, J=8.8 Hz, 1H), 8.84 (dd, J=4.4, 1.2 Hz, 1H), 7.90 (d, J=9.2, 1H), 7.86 (dd, J=7.6, 1.2 Hz, 1H), 7.79 (dd, J=8.0, 1.6 Hz, 1H), 7.66 (td, J=6.8, 1.2 Hz, 1H), 7.57 (dd, J=8.0, 4.4 Hz, 1H), 7.42 (m, 4H), 7.34 (m, 3H), 7.19 (d, J=9.2 Hz, 1H).

Compound 230: 2-Methoxy-6-[3-(3-phenyl-pyridin-2-yl)-[1,2,4]oxadiazol-5-yl]-phenol

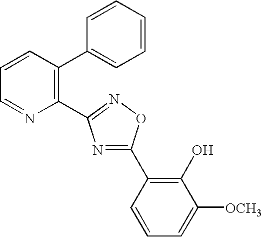

The synthesis was carried out with N-Hydroxy-3-phenyl-pyridine-2-carboxamidine (O35) and 3-methoxysalicylic acid using the protocol detailed in Example 8 to produce 16 mg of beige powder in 18% yield. [M H]⁺: 346.2; ¹H NMR (CDCl₃) δ 9.62 (s, 1H), 8.79 (dd, J=4.4, 1.2 Hz, 1H), 7.83 (dd, J=8.4, 2.0 Hz, 1H), 7.53 (dd, J=7.6, 4.8 hz, 1H), 7.52 (dd, J=8.0, 1.6 Hz, 1H), 7.37 (m, 3H), 7.27 (m, 2H), 7.02 (dd, J=8.0, 1.2 Hz, 1H), 6.91 (t, J=8.4 Hz, 1H), 3.89 (s, 3H).

Compound 231: 5-Dimethylamino-2-[3-(3-phenyl-pyridin-2-yl)-[1,2,4]oxadiazol-5-yl]-phenol

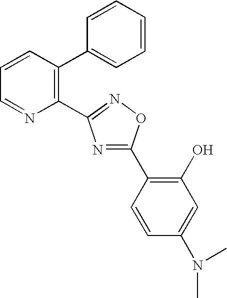

The synthesis was carried out with N-Hydroxy-3-phenyl-pyridine-2-carboxamidine (O35) and 4-dimethylaminosalicylic acid using the protocol detailed in Example 8 to produce 5 mg of beige powder in 9% yield. [M H]⁺: 359.3; ¹H NMR (CDCl₃) δ 9.50 (s, 1H), 8.80 (dd, J=4.8, 1.6 Hz, 1H), 7.81 (dd, J=8.0, 2.0 Hz, 1H), 7.71 (d, (J=8.8 Hz, 1H), 7.52 (dd, J=8.0, 4.4 Hz, 1H), 7.39 (m, 3H), 7.31 (m, 2H), 6.32 (dd, J=8.8, 2.8 Hz, 1H), 6.18 (d, J=2.4 Hz, 1H), 3.03 (s, 6H).

Compound 232: 4-Methyl-2-[3-(3-phenyl-pyridin-2-yl)-[1,2,4]oxadiazol-5-yl]-phenol

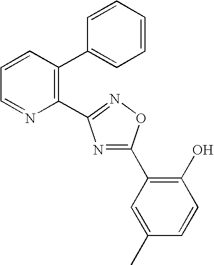

The synthesis was carried out with N-Hydroxy-3-phenyl-pyridine-2-carboxamidine (O35) and 5-methylsalicylic acid using the protocol detailed in Example 8 to produce 37 mg of beige powder in 48% yield. [M H]⁺: 330.2; ¹H NMR (CDCl₃) δ 9.25 (s, 1H), 8.81 (dd, J=4.8, 1.6 Hz, 1H), 7.70 (m, 1H), 7.54 (dd, J=8.0, 4.4 Hz, 1H), 7.39 (m, 3H), 7.28 (m, 2H), 7.25 (dd, J=8.4, 2.4 Hz, 1H), 6.90 (d, J=8.8 Hz, 1H), 2.32 (s, 3H).

Compound 233: 3,5-Dimethoxy-2-[3-(3-phenyl-pyridin-2-yl)-[1 ,2,4]oxadiazol-5-yl]-phenol

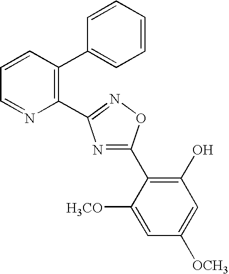

The synthesis was carried out with N-Hydroxy-3-phenyl-pyridine-2-carboxamidine and 4,6-dimethoxysalicylic acid using the protocol detailed in Example 8 to produce 6 mg of brown oily solid in 8% yield. [M H]⁺: 376.2; ¹H NMR (CDCl₃) δ 10.90 (s, 1H), 8.80 (dd, J=4.0, 1.2 Hz, 1H), 7.82 (dd, J=8.0, 1.6 Hz, 1H), 7.52 (dd, J=7.2, 4.8 Hz, 1H), 7.38 (m, 3H), 7.29 (m, 2H), 6.15 (d, J=2.0 Hz, 1H), 6.04 (d, J=2.4 Hz, 1H), 3.92 (s, 3H), 3.82 (s, 3H).

Compound 234: 5-Ethoxy-2-[3-(3-phenyl-pyridin-2-yl)-[1,2,4]oxadiazol-5-yl]-phenol

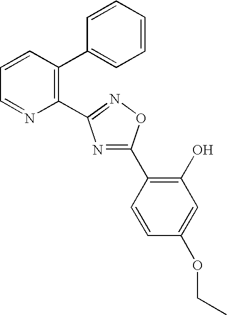

The synthesis was carried out with N-Hydroxy-3-phenyl-pyridine-2-carboxamidine and 4-ethoxysalicylic acid using the protocol detailed in Example 8 to produce 12 mg of beige oily solid in 15% yield. [M H]⁺: 360.2; ¹H NMR (CDCl₃) δ 9.60 (s, 1H), 8.80 (dd, J=4.8, 1.6 Hz, 1H), 7.82 (dd, J=7.6, 1.2 Hz, 1H), 7.79 (d, J=8.8 Hz, 1H), 7.53 (dd, J=7.6, 4.4 Hz, 1H), 7.39 (m, 3H), 7.29 (m, 2H), 6.62 (dd, J=8.8, 2.0 Hz, 1H), 6.47 (d, J=2.0 Hz, 1H), 4.05 (q, J=7.2 Hz, 2H), 1.43 (t, J=7.2 Hz, 3H).

Compound 235: 5-Methoxy-2-[3-(3-phenyl-pyridin-2-yl)-[1,2,4]oxadiazol-5-yl]-phenol

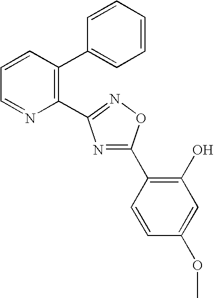

The synthesis was carried out with N-Hydroxy-3-phenyl-pyridine-2-carboxamidine (O35) and 4-methoxysalicylic acid using the protocol detailed in Example 8 to produce 24 mg of brown oily solid in 27% yield. [M H]⁺: 346.2.

Compound 236: 2-[3-(3-Phenyl-pyridin-2-yl)-[1,2,4]oxadiazol-5-yl]-benzene-1,4-diol

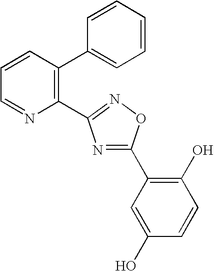

The synthesis was carried out with N-Hydroxy-3-phenyl-pyridine-2-carboxamidine and 2,5-dihydroxybenzoic acid using the protocol detailed in Example 8 to produce 9 mg of yellow solid in 11% yield. [M H]⁺: 332.2; ¹H NMR (CDCl₃) δ 9.01 (s, 1H), 8.80 (dd, J=4.4, 1.6 Hz, 1H), 7.85 (dd, J=8.0, 2.0 Hz, 1H), 7.39 (m, 3H), 7.35 (d, J=3.2 Hz, 1H), 7.28 (m, 2H), 6.99 (dd, J=8.8, 2.8 Hz, 1H), 6.88 (d, J=8.8 Hz, 1H).

Compound 244: {6-[5-(5-Bromo-2-hydroxy-phenyl)-[1,2,4]oxadiazol-3-yl]-pyridin-3-yl}-carbamic acid tert-butyl ester

### EXAMPLE 28

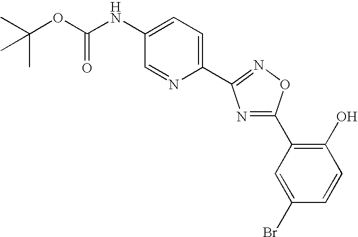

Step 1: Preparation of [6-(N-Hydroxycarbamimidoyl)-pyridin-3-yl]-carbamic acid tert-butyl ester

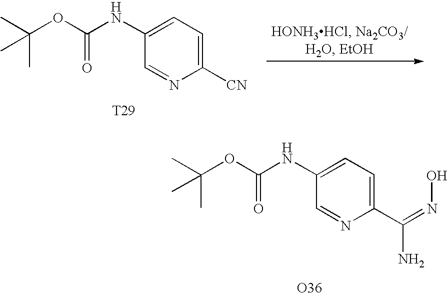

(6-Cyano-pyridin-3-yl)-carbamic acid tert-butyl ester (T29) (1.3 g, 6.7 nmroles) was subjected to conditions detailed in example 8 to produce 978 mg of solid product in 58% yield. [M H]⁺: 253.3; ¹H NMR (d₆-DMSO) δ 9.68 (s, 1H), 9.65 (s, 1H), 8.57 (m, 1H), 7.87 (m, 1H), 7.72 (d, J=8.8 Hz, 1H), 5.70 (br s, 2 H), 1.48 (s, 9H).

Step 2

Compound 244: {6-[5-(5-Bromo-2-hydroxy-phenyl)-[1,2,4]oxadiazol-3-yl]-pyridin-3-yl}-carbamic acid tert-butyl ester

[6-(N-Hydroxycarbamimidoyl)-pyridin-3-yl]-carbamic acid tert-butyl ester (O36) (200 mg, 0.80 mmoles) and 5-bromosalicylic acid (139 mg, 0.80 mmoles) were subjected to the conditions detailed in example 8 to produce 17 mg of white powder in 5% yield. [M H]⁺: 433.2, 435.2; ¹H NMR (CDCl₃) δ 8.56 (d, J=2.0 Hz, 1H), 8.26 (d, J=8.8 Hz, 1H), 8.13 (d, J=2.4 Hz, 1H), 8.08 (d, J=8.4 Hz, 1H), 7.59 (dd, J=8.8, 2.4 Hz, 1H), 7.05 (d, J=8.8 Hz, 1H), 6.78 (br s, 2H), 1.55 (s, 9H).

Compound 254: 2-[3-(5-Amino-pyridin-2-yl)-[1,2,4]oxadiazol-5-yl]-4-bromo-phenol

### EXAMPLE 29

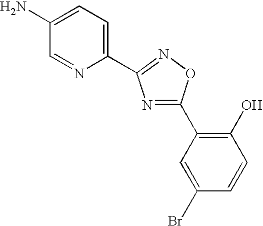

{6-[5-(5-Bromo-2-hydroxy-phenyl)-[1,2,4]oxadiazol-3-yl]-pyridin-3-yl} -carbamic acid tert-butyl ester (244) (11 mg, 0.03 mmoles) was dissolved in dichloromethane (500 μl). Trifluoroacetic acid (500 μl) was then added. The mixture stirred for 30 m at ambient temperature, and was then concentrated in vacuo. The residue was then immediately purified by chromatography to produce 15 mg of yellow solid in quantitative yield. [M H]⁺: 333.0, 335.0.

Compound 263: N-{6-[5-(5-Bromo-2-hydroxy-phenyl)-[1,2,4]oxadiazol-3-yl]-pyridin-3-yl}-acetamide

### EXAMPLE 30

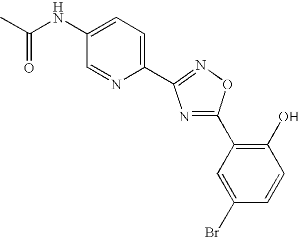

2-[3-(5-Amino-pyridin-2-yl)-[1,2,4]oxadiazol-5-yl]-4-bromo-phenol (254) (10 mg, 0.03 mmoles) and acetic anhydride (2.5 equiv, 7 μl, 0.07 mmoles) were dissolved in N,N-dimethylformamide (500 μl). Diisopropylethylamine (7 equiv., 39 μl, 0.22 mmoles) were then added. The mixture stirred at ambient temperature for 15 hours, and was then concentrated in vacuo. The residue was then purified by chromatography to produce 10 mg of yellow powder in 89% yield. [M H]⁺: 375.3, 377.3.

Compound 245: {6-[5-(2-Hydroxy-5-methyl-phenyl)-[1,2,4]oxadiazol-3-yl]-pyridin-3-yl}-carbamic acid tert-butyl ester

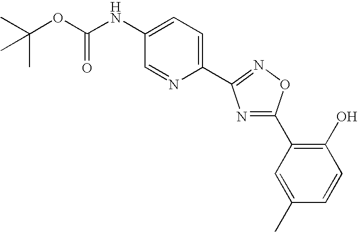

[6-(N-Hydroxycarbamimidoyl)-pyridin-3-yl]-carbamic acid tert-butyl ester (O36) (200 mg, 0.80 mmoles) and 5-metylsalicylic acid (122 mg, 0.80 mmoles) were subjected to the conditions detailed in example 8 to produce 18 mg of white powder in 8% yield. [M H]⁺: 369.3; ¹H NMR (CDCl₃) δ 10.20 (s, 1H), 8.57 (d, J=3.2 Hz, 1H), 8.26 (d, J=7.6 Hz, 1H), 8.09 (d, J=8.4 Hz, 1H), 7.82 (s, 1H), 7.33 (dd, J=8.4, 2.0 Hz, 1H), 7.05 (d, J=8.4 hz, 1H), 2.38 (s, 3H).

Compound 255: 2-[3-(5-Amino-pyridin-2-yl)-[1,2,4]oxadiazol-5-yl]-4-methyl-phenol

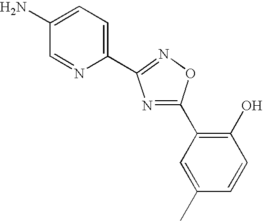

{6-[5-(2-Hydroxy-5-methyl-phenyl)-[1,2,4]oxadiazol-3-yl]-pyridin-3-yl} -carbamic acid tert-butyl ester (245) (16 mg, 0.04 mmoles) was subjected to the procedures detailed in example 29 to produce 12 mg of a white powder in quantitative yield. [M H]⁺: 269.1; ¹H NMR (CDCl₃) δ 8.26 (d, J=2.8 Hz, 1H), 7.93 (d, J=8.4 Hz, 1H), 7.81 (s, 1H), 7.32 ( dd, J=8.4, 2.0 Hz, 1H), 7.08 (dd, J=8.4, 2.8 Hz, 1H), 7.04 (d, J=8.4 Hz, 1H), 4.10 (br s, 2H), 2.38 (s, 3H).

Compound 264: N-{6-[5-(2-Hydroxy-5-methyl-phenyl)-[1,2,4]oxadiazol-3-yl]-pyridin-3-yl}-acetamide

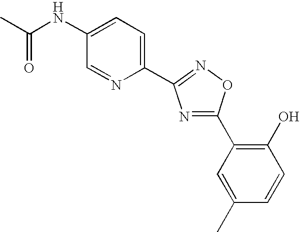

2-[3-(5-Amino-pyridin-2-yl)-[1,2,4]oxadiazol-5-yl]-4-methyl-phenol (255) (10 mg, 0.04 mmoles) was subjected to the procedures detailed in example 30 to produce 6 mg of a white powder in 52% yield. [M H]⁺: 311.3, ¹H NMR (CDCl₃) δ 8.69 (d, J=2.4 Hz, 1H), 8.51 (dd, J=8.8, 2.0 Hz, 1H), 8.15 (s, 1H), 8.12 (s, 1H), 7.86 (br s, 1H), 7.43 (dd, 8.0, 1.6 Hz, 1H), 7.13 (d, J=8.4 Hz, 1H), 2.42 (s, 3H), 2.24 (s, 3H).

Compound 237: {6-[5-(2-Hydroxy-3-methoxy-phenyl)-[1,2,4]oxadiazol-3-yl]-pyridin-3-yl}-carbamic acid tert-butyl ester

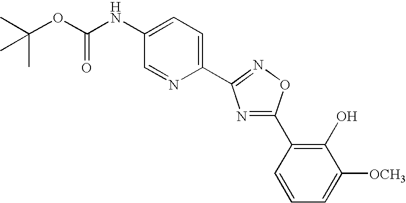

[6-(N-Hydroxycarbamimidoyl)-pyridin-3-yl]-carbamic acid tert-butyl ester (O36) (200 mg, 0.80 mmoles) and 3-methoxysalicylic acid (135 mg, 0.80 mmoles) were subjected to the conditions detailed in example 8 to produce 13 mg of white powder in 6% yield. [M H]⁺: 385.3; ¹H NMR (CDCl₃) δ 10.50 (s, 1H), 8.57 (d, J=2.4 Hz, 1H), 8.26 (d, J=8.4 Hz, 1H), 8.07 (d, J=8.4 Hz, 1H), 7.62 (dd, J=8.0, 1.6 Hz, 1H), 7.10 (dd, J=8.0, 1.6 Hz, 1H), 6.99 (t, J=8.0 Hz, 1H), 6.80 (s, 1H)3.96 (s, 3H), 1.56 (s, 9H).

Compound 251: 2-[3-(5-Amino-pyridin-2-yl)-[1,2,4]oxadiazol-5-yl]-6-methoxy-phenol

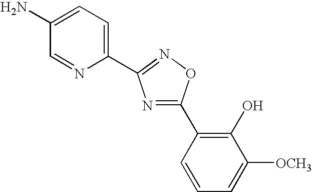

{6-[5-(2-Hydroxy-3-methoxy-phenyl)-[1,2,4]oxadiazol-3-yl]-pyridin-3-yl} -carbamic acid tert-butyl ester (237) (11 mg, 0.03 mmoles) was subjected to the procedures detailed in example 29 to produce 9 mg of a white powder in quantitative yield. [M H]⁺: 285. 1; ¹H NMR (d₄-CD₃OD) δ 8.09 (d, J=2.4 Hz, 1H), 7.93 (d, J=8.4 Hz, 1H), 7.59 (dd, J=8.0, 1.2 Hz, 1H), 7.22 (dd, J=8.0, 1.2 Hz, 1H), 7.15 (dd, J=8.4, 3.2 Hz, 1H), 7.01 (t, J=8.0 Hz, 1H), 3.93 (s, 3H).

Compound 260: N-{6-[5-(2-Hydroxy-3-methoxy-phenyl)-[1,2,4]oxadiazol-3-yl]-pyridin-3-yl}-acetamide

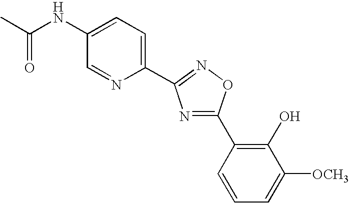

2-[3-(5-Amino-pyridin-2-yl)-[1,2,4]oxadiazol-5-yl]-6-methoxy-phenol (251) (9 mg, 0.03 mmoles) was subjected to the procedures detailed in example 30 to produce 4 mg of a yellow-white powder in 35% yield. [M H]⁺: 327.3; ¹H NMR (CDCl₃) δ 8.66 (s, 1H), 8.47 (d, J=7.6 Hz, 1H), 8.11 (d, J=8.4 Hz, 1H), 8.08 (s, 1H), 7.86 (dd, J=8.4, 1.2 Hz, 1H), 7.37 (t, J=8.0 Hz, 1H), 7.21 (d, J=8.4 Hz, 1H), 3.91 (s, 3H), 2.23 (s, 3H).

Compound 238: {6-[5-(2-Hydroxy-4-methoxy-phenyl)-[1,2,4]oxadiazol-3-yl]-pyridin-3-yl}-carbamic acid tert-butyl ester

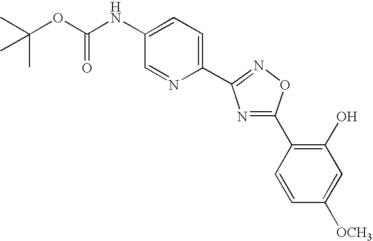

[6-(N-Hydroxycarbamimidoyl)-pyridin-3-yl]-carbamic acid tert-butyl ester (O36) (200 mg, 0.80 mmoles) and 4-methoxysalicylic acid (135 mg, 0.80 mmoles) were subjected to the conditions detailed in example 8 to produce 16 mg of white powder in 7% yield. [M H]⁺: 385.3; ¹H NMR (CDCl₃) δ 8.10.55 (s, 1H), 8.56 (d, J=2.4 Hz, 1H), 8.25 (d, J=7.2 Hz, 1H), 8.07 (d, J=8.8 Hz, 1H), 7.91 (d, J=8.4 Hz, 1H), 6.82 (br s, 1H), 6.62 (s, 1H), 6.61 (dd, J=6.8, 2.4 Hz, 1H), 3.88 (s, 3H), 1.55 (s, 9H).

Compound 253: 2-[3-(5-Amino-pyridin-2-yl)-[1,2,4]oxadiazol-5-yl]-6-methoxy-phenol

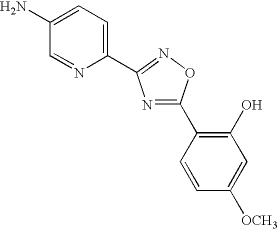

{6-[5-(2-Hydroxy-4-methoxy-phenyl)-[1,2,4]oxadiazol-3-yl]-pyridin-3-yl}-carbamic acid tert-butyl ester (14 mg, 0.04 mmoles) was subjected to the conditions detailed in example XX to produce 10 mg of white powder in 88% yield. [M H]⁺: 285.2 Compound 261: N-{6-[5-(2-Hydroxy-4-methoxy-phenyl)-[1,2,4]oxadiazol-3-yl]-pyridin-3-yl}-acetamide

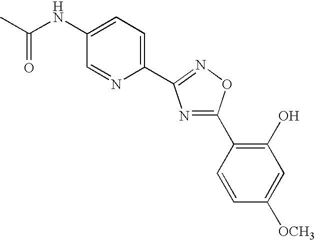

2-[3-(5-Amino-pyridin-2-yl)-[1,2,4]oxadiazol-5-yl]-6-methoxy-phenol (253) (10 mg, 0.04 mmoles) was subjected to the conditions detailed in example 30 to produce 11 mg of yellow powder in 88% yield. [M H]⁺: 327.4; ¹H NMR (CDCl₃) δ 9.57 (br s, 1H), 9.04 (s, 1H), 8.86 (d, J=7.6 Hz, 1H), 8.16 (m, 2H), 6.85 (dd, J=9.2, 2.4 Hz, 1H), 8.66 (d, J=2.4 Hz, 1H), 3.82 (s, 3H), 2.42 (s, 3H).

Compound 239: {6-[5-(2-Hydroxy-5-methoxy-phenyl)-[1,2,4]oxadiazol-3-yl]-pyridin-3-yl}-carbamic acid tert-butyl ester

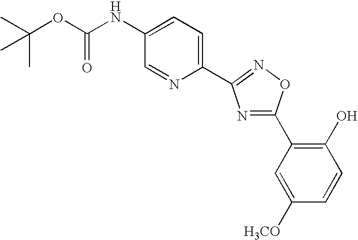

[6-(N-Hydroxycarbamimidoyl)-pyridin-3-yl]-carbamic acid tert-butyl ester (O36) (200 mg, 0.80 mmoles) and 5-methoxysalicylic acid (135 mg, 0.80 mmoles) were subjected to the conditions detailed in example 8 to produce 19 mg of white powder in 8% yield. [M H]⁺: 385.3; ¹H NMR (CDCl₃) δ 10.03 (s, 1H), 8.56 (d, J=2.8 Hz, 1H), 8.26 (d, J=8.0 Hz, 1H), 8.08 (d, J=8.8 Hz, 1H), 7.44 (d, J=2.8 Hz, 1H), 7.14 (dd, J=9.2, 3.6 Hz, 1H), 7.08 (d, J=8.8 Hz, 1H), 6.78 (br s, 1H), 3.68 (s, 3H), 1.56 (s, 9H).

Compound 252: 2-[3-(5-Amino-pyridin-2-yl)-[1,2,4]oxadiazol-5-yl]-4-methoxy-phenol

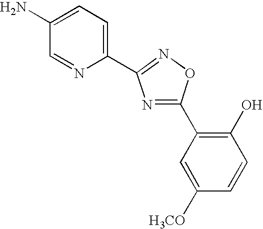

{6-[5-(2-Hydroxy-5-methoxy-phenyl)-[1,2,4]oxadiazol-3-yl]-pyridin-3-yl}-carbamic acid tert-butyl ester (239) (17 mg, 0.04 mmoles) was subjected to the conditions detailed in example 8 to produce 16 mg of white powder in quantitative yield. [M H]⁺: 285.3.

Compound 262: N-{6-[5-(2-Hydroxy-5-methoxy-phenyl)-[1,2,4]oxadiazol-3-yl]-pyridin-3-yl}-acetamide

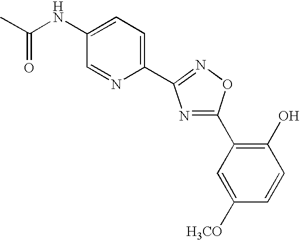

2-[3-(5-Amino-pyridin-2-yl)-[1,2,4]oxadiazol-5-yl]-4-methoxy-phenol (252) (10 mg, 0.04 mmoles) was subjected to the conditions detailed in example 30 to produce 8 mg of yellow powder in 67% yield. [M H]⁺: 327.3; ¹H NMR (CDCl₃) δ 8.70 (s, 1H), 8.51 (d, J=9.6 Hz, 1H), 8.15 (d, J=8.8 Hz, 1H), 7.791 (t, J=1.6 Hz, 1H), 7.68 (s, 1H), 7.16 (d, J=1.6 Hz, 1H), 3.91, (s, 3H), 2.28 (s, 3H).

Compound 193: 1-[5-(2,5-Dimethoxy-phenyl)-[1,2,4]oxadiazol-3-yl]-isoquinoline

### EXAMPLE 31

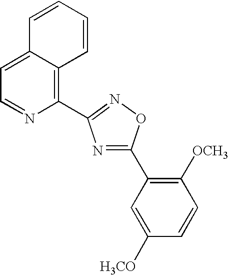

Step 1: Preparation of N-Hydroxy-isoguinoline-1-carboxamidine (O37)

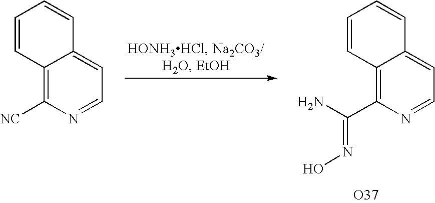

1-isoquinolinecarbonitrile was subjected to the conditions outlined in example 1 to produce 8.2 g of white powder in 96% yield. [M+H]⁺ 187.9; ¹H (CDCl₃) δ 9.14 (dd, J=8.4, 0.8 Hz, 1H), 8.51 (d, J=5.2 Hz, 1H), 8.2 (b, 1H), 7.82 (d, 7.2 Hz, 1H), 7.66 (m, 3H), 5.45 (s,2H).

Step 2

Compound 193: 1-[5-(2,5-Dimethoxy-phenyl)-[1,2,4]oxadiazol-3-yl]-isoquinoline

N-Hydroxy-isoquinoline-1-carboxamidine (O37) and 2,5-dimethoxybenzoic acid was subjected to the conditions outlined in example 1 to produce 72 mg of desired product in 41% yield. [M+H]⁺ 334.3; ¹H (CDCl₃) δ 9.04 (d, J=8.4 Hz, 1H), 8.79 (d, J=5.6 Hz, 1H), 7.93 (d, J=8.0 Hz, 1H), 7.85 (d, J=5.6 Hz, 1H), 7.76 (m, 3H), 7.15 (dd, J=9.2, 3.6 Hz, 1H), 7.05 (d, J=9.2 Hz, 1H), 4.03 (s, 3H), 3.88 (s, 3H).

Compound 210: 1-(3-Isoquinolin-1-yl-[1,2,4]oxadiazol-5-yl)-naphthalen-2-ol

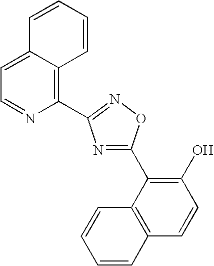

N-Hydroxy-isoquinoline-1-carboxamidine (O37) and 2-hydroxy-1-naphthoic acid were subjected to the conditions outlined in example 8 to produce 18 mg of desired product in 10% yield. [M+H]⁺: 340.1; ¹H (CDCl₃) δ 9.01 (d, J=8.8 Hz, 1H), 8.95 (d, J=8.8 Hz, 1H), 8.83 (d, J=5.6 Hz, 1H), 8.00 (d, J=8.8 Hz, 2H), 7.94 (d, J=5.2 Hz, 1H), 7.80 (m, 4H), 7.49 (t, J=7.6 Hz, 1H), 7.36 (d, J=9.2 Hz, 1H).

Compound 194: 2-(3-Isoquinolin-1-yl-[1,2,4]oxadiazol-5-yl)-4-methyl-phenol

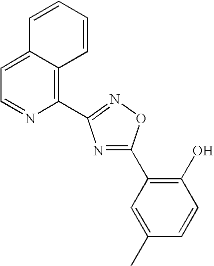

N-Hydroxy-isoquinoline-1-carboxamidine (O37) and 2,5-dimethoxybenzoic acid were subjected to the conditions outlined in example 8 to produce 35 mg of desired product in 21% yield. [M+H]⁺ 304.2.

Compound 195: 2-(3-Isoquinolin-1-yl-[1,2,4]oxadiazol-5-yl)-4-nitro-phenol

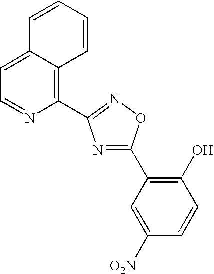

N-Hydroxy-isoquinoline-1-carboxamidine (O37) and 5-nitrosalicylic acid were subjected to the conditions outlined in example 8 to produce 8 mg of desired product in 5% yield. [M+H]⁺ 335.1; ¹H (CDCl₃) δ 9.04 (d, J=2.4 Hz, 1H), 8.94 (d, J=8.8 Hz, 1H), 8.85 (d, J=5.6 Hz, 1H), 8.43 (dd, J=9.2, 2.8 Hz, 1H), 8.01 (d, J=8.0 Hz, 1H), 7.97 (d, J=5.6 Hz, 1H), 7.84 (m, 2H), 7.31 (d, J=9.2 Hz, 1H).

Compound 196: 2-(3-Isoquinolin-1-yl-[1,2,4]oxadiazol-5-yl)-benzene-1,4-diol

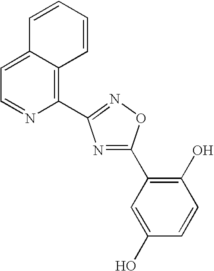

N-Hydroxy-isoquinoline-1-carboxamidine (O37) and 2,5-dihydroxybenzoic acid were subjected to the conditions outlined in example 8 to produce 35 mg of desired product in 22% yield. [M+H]⁺ 306.2.

Compound 197: 1-[5-(3-Methoxy-phenyl)-[1,2,4]oxadiazol-3-yl]-isoquinoline

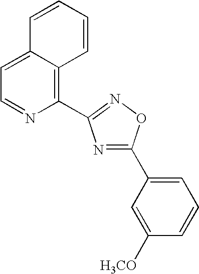

N-Hydroxy-isoquinoline-1-carboxamidine (O37) and 3-methoxybenzoic acid were subjected to the conditions outlined in example 8 to produce 54 mg of desired product in 34% yield. [M+H]⁺ 304.3.

Compound 198: 4-Chloro-2-(3-isoquinolin-1-yl-[1,2,4]oxadiazol-5-yl)-phenol

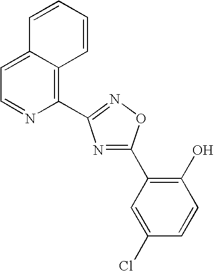

N-Hydroxy-isoquinoline-1-carboxamidine and 5-chlorosalicylic acid were subjected to the conditions outlined in example 8 to produce 32 mg of desired product in 19% yield. [M+H]⁺ 324.0.

Compound 199: 4-Fluoro-2-(3-isoquinolin-1-yl-[1,2,4]oxadiazol-5-yl)-phenol

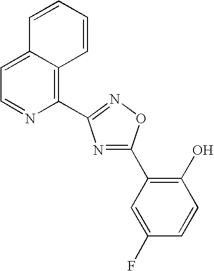

N-Hydroxy-isoquinoline-1-carboxamidine (O37) and 5-fluorosalicylic acid were subjected to the conditions outlined in example 8 to produce 27 mg of desired product in 17% yield. [M+H]⁺ 308.1.

Compound 200: 1-(5-Naphthalen-1-yl-[1,2,4]oxadiazol-3-yl)-isoquinoline

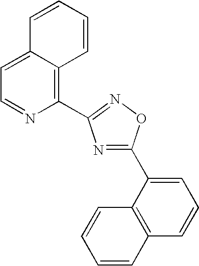

N-Hydroxy-isoquinoline-1-carboxamidine (O37) and l-naphthoic acid were subjected to the conditions outlined in example 8 to produce 77 mg of desired product in 45% yield. [M+H]⁺: 324.2.

Compound 201: 5-Dimethylamino-2-(3-isoquinolin-1-yl-[1,2,4]oxadiazol-5-yl)-phenol

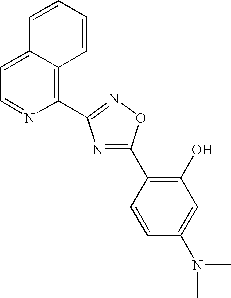

N-Hydroxy-isoquinoline-1-carboxamidine (O37) and 4-dimethylaminosalicylic acid were subjected to the conditions outlined in example 8 to produce 12 mg of desired product in 7% yield. [M+H]⁺: 333.3.

Compound 202: 2-(3-Isoquinolin-1-yl-[1,2,4]oxadiazol-5-yl)-4-methoxy-phenol

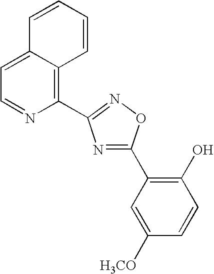

N-Hydroxy-isoquinoline-1-carboxamidine (O37) and 4-dimethylaminosalicylic acid were subjected to the conditions outlined in example 8 to produce 47 mg of desired product in 28% yield. [M+H]⁺: 320.2.

Compound 203: 1-[5-(2-Methoxy-naphthalen-1-yl)-[1,2,4]oxadiazol-3-yl]-isoquinoline

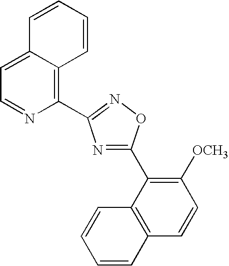

N-Hydroxy-isoquinoline-1-carboxamidine (O37) and 2-methoxy-1-naphtoic acid were subjected to the conditions outlined in example 8 to produce 8 mg of desired product in 4% yield. [M+H]⁺: 354.2.

Compound 204: 4-Bromo-2-(3-isoquinolin-1-yl-[1,2,4Joxadiazol-5-yl)-phenol

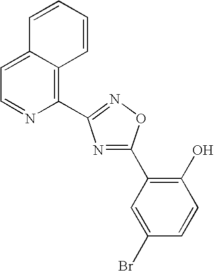

N-Hydroxy-isoquinoline-1-carboxamidine and 5-bromosalicylic acid were subjected to the conditions outlined in example 8 to produce 46 mg of desired product in 24% yield. [M+H]⁺: 368.1, 370.1.

Compound 205: 2-(3-Isoquinolin-1-yl-[1,2,4]oxadiazol-5-yl)-5-methoxy-phenol

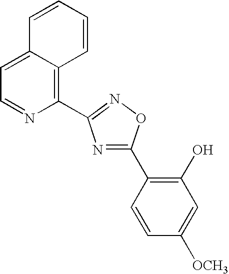

N-Hydroxy-isoquinoline-1-carboxamidine and 4-methoxysalicylic acid were subjected to the conditions outlined in example 8 to produce 31 mg of desired product in 18% yield. [M+H]⁺: 320.2.

Compound 206: 2-(3-Isoquinolin-1-yl-[1,2,4Joxadiazol-5-yl)-phenol

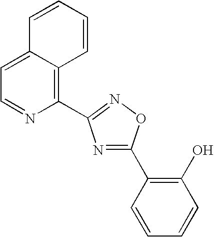

N-Hydroxy-isoquinoline-1-carboxamidine and salicylic acid were subjected to the conditions outlined in example 8 to produce 62 mg of desired product in 40% yield. [M+H]⁺: 290.1.

Compound 209: 2-(3-Isoquinolin-1-yl-[1,2,4]oxadiazol-5-yl)-6-methoxy-phenol

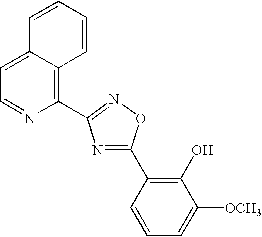

N-Hydroxy-isoquinoline-1-carboxamidine (O37) and 3-methoxysalicylic acid were subjected to the conditions outlined in example 8 to produce 20 mg of desired product in 12% yield. [M+H]⁺: 320.1.

Compound 212: 1-15-(2-Chloro-4-nitro-phenyl)-[1,2,4]oxadiazol-3-yl]-isoquinoline

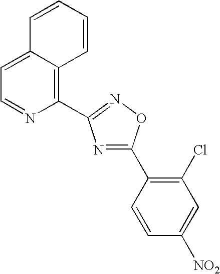

N-Hydroxy-isoquinoline-1-carboxamidine (O37) and 2-chloro-4-nitrobenzoic acid were subjected to the conditions outlined in example 8 to produce 23 mg of desired product in 12% yield. [M+H]⁺: 353.2.

Compound 283: 2-(3-Furan-3-yl-[1,2,4]oxadiazol-5-yl)-4-methoxy-phenol

### EXAMPLE 32

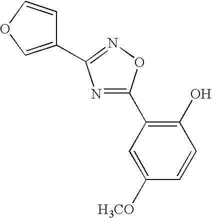

Step 1: Preparation of N-Hydroxy-furan-3-carboxamidine

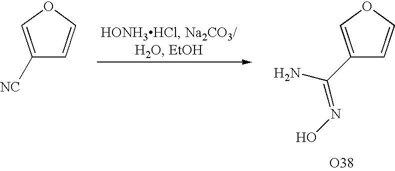

3-furonitrile (415 mg, 4.4 mmoles) was subjected to the conditions described in example 8 to produce 480 mg of brown oily solid in 86% yield.

Step 2

Compound 283: 2-(3-Furan-3-yl-[1,2,4]oxadiazol-5-yl)-4-methoxy-phenol

N-Hydroxy-furan-3-carboxamidine (O38) (96 mg, 0.76 mmoles) and 5-methoxysalicylic acid (1.0 equiv, 128 mg, 0.76 mmoles) were subjected to the conditions described in example 8 to produce 5 mg of white powder in 3% yield. [M+H]⁺: 259.2; ¹H NMR (CDCl₃) δ 8.16 (m, 1H), 7.57 (m, 1H), 7.41 (d, J=3.2 Hz, 1H), 7.14 (dd, J=9.2, 3.2 Hz, 1H), 7.07 (d, J=9.2 Hz, 1H), 6.95 (m, 1H), 3.86 (s, 3H).

Compound 284: 2-(3-Furan-3-yl-[1,2,4]oxadiazol-5-yl)-4-methyl-phenol

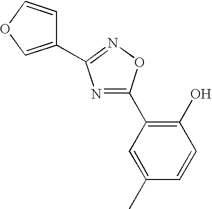

N-Hydroxy-furan-3-carboxamidine (O38) (96 mg, 0.76 mmoles) and 5-methylsalicylic acid (1.0 equiv, 116 mg, 0.76 mmoles) were subjected to the conditions described in example 8 to produce 5 mg of white powder in 3% yield. [M+H]⁺: 259.2; ¹H NMR (CDCl₃) δ 8.14 (m, 1H), 7.76 (m, 1H), 7.55 (m, 1H), 7.32 (dd, J=8.8, 2.4 Hz, 1H), 7.02 (d, J=8.4 Hz, 1H), 6.93 (m, 1H)2.37 (s, 3H).

Compound 285: 2-(3-Furan-3-yl-[1,2,4]oxadiazol-5-yl)-naphthalen-1-ol

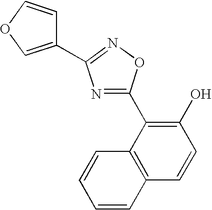

N-Hydroxy-furan-3-carboxamidine (O38) (96 mg, 0.76 mmoles) and 2-hydroxy-1-naphthoic acid (1.0 equiv, 143 mg, 0.76 mmoles) were subjected to the conditions described in example 8 to produce 3 mg of white powder in 2% yield. ¹H NMR (CDCl₃) δ 8.93 (dd, J=8.8, 0.8 Hz, 1H), 8.21 (q, J=0.8 Hz, 1H), 7.98 (d, J=8.8 Hz, 1H), 7.85 (d, J=8.0, 1H), 7.70 (td, J=8.4, 1.2 Hz, 1H), 7.60 (t, J=1.6 Hz, 1H), 7.47 (td, J=8.0, 0.8 Hz, 1H), 7.32 (d, J=8.8 Hz, 1H), 7.00 (q, J=0.8Hz, 1H).

Compound 286: 2-(3-Furan-3-yl-[1,2,4]oxadiazol-5-yl)-5-methoxy-phenol

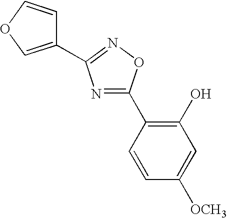

N-Hydroxy-furan-3-carboxamidine (O38) (96 mg, 0.76 mmoles) and 4-methoxysalicylic acid 1.0 equiv, 128 mg, 0.76 mmoles) were subjected to the conditions described in example 8 to produce 5 mg of white powder in 3% yield. [M H]⁺: 259.2; ¹H NMR (CDCl₃) δ 10.43 (s, 1H), 8.06 (s, 1H), 7.48 (m, 2H), 7.01 (d, J=8.0 Hz, 1H), 6.88 (m, 2H), 3.85 (s, 3H).

Compound 301: 4-Methoxy-2-[5-(3-methyl-pyridin-2-yl)-[1,2,4]oxadiazol-3-yl]-phenol

### EXAMPLE 33

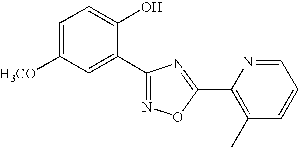

Step 1: Preparation of 2.N-Dihydroxy-5-methoxy-benzamidine (O39)

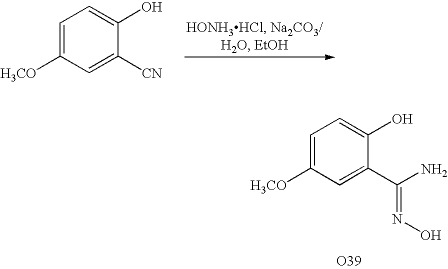

2-Hydroxy-5-methoxy-benzonitrile (250 mg, 1.7 mmoles) was subjected to the conditions described in Example 1 to produce 250 mg of brown powder in 81% yield.

Step 2

Compound 301: 4-Methoxy-2-[5-(3-methyl-pyridin-2-yl)-[1,2,4]oxadiazol-3-yl]-phenol

2,N-Dihydroxy-5-methoxy-benzamidine (O39) (83 mg, 0.46 mmoles) and 3-methylpicolinic acid (1.0 equiv, 63 mg, 0.46 mmoles) was subjected to the conditions detailed in example 8 to produce 11 mg of yellow powder in 8% yield. [M H]⁺: 284.2; ¹H NMR (CDCl₃) δ 8.51 (dd, J=4.4, 0.8 Hz, 1H), 7.72 (d, J=7.2 Hz, 1H), 7.46 (dd, J=7.6, 4.4 Hz, 1H), 7.39 (d, J=8.8 Hz, 1H), 7.16 (d, J=2.4 Hz, 1H), 6.84 (dd, J=8.8, 2.4 Hz, 1H), 3.86 (s, 3H).

Compound 299: 2-(5-Furan-3-yl-[1,2,4]oxadiazol-3-yl)-4-methoxy-phenol

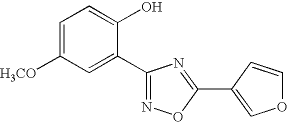

2,N-Dihydroxy-5-methoxy-benzamidine (O39) (83 mg, 0.46 mmoles) and 3-furoic acid (1.0 equiv, 52 mg, 0.46 mmoles) was subjected to the conditions described in example 8 to produce 5 mg of yellow powder in 4% yield. [M H]⁺: 259.2; ¹H NMR (CDCl₃) δ 8.22 (s, 1h), 7.46 (t, J=1.6 Hz, 1H), 7.34 (d, J=8.8 Hz, 1H), 6.88 (m, 2H), 6.82 (dd, J=9.2, 2.4 Hz, 1H), 3.83 (s, 3H).

Compound 302: 2-(5-Furan-2-yl-[1,2,4]oxadiazol-3-yl)-4-methoxy-phenol

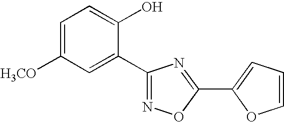

2,N-Dihydroxy-5-methoxy-benzamidine (O39) (83 mg, 0.46 mmoles) and 2-furoic acid (1.0 equiv, 52 mg, 0.46 mmoles) was subjected to the conditions described in Example 8 to produce 10 mg of yellow powder in 8% yield. [M H]⁺: 259.2; ¹H NMR (CDCl₃) δ 7.59 (s, 1H), 7.42 (dd, J=3.6, 0.8 Hz, 1H), 7.38 (d, J=8.8 Hz, 1H), 7.01 (d, J=2.4 Hz, 1H), 6.85 (dd, J=9.2, 2.8 Hz, 1H), 6.59 (dd, J=36, 2.0 Hz, 1H), 4.85 (s, 3H).

Compound 136: 1-[3-(2-Fluoro-5-trifluoromethyl-phenyl)-[1,2,4]oxadiazol-5-yl]-naphthalen-2-ol

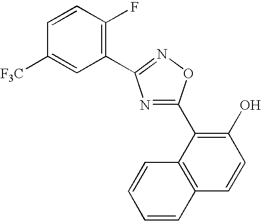

2-fluoro-5-(trifluoromethyl)benzoic acid and 2-hydroxy-1-naphthoic acid were subjected to protocol outlined in Example 2 to produce the desired product. ¹H (400 MHz, d₆-DMSO) δ 11.08 (br s, 1H), 8.40 (t, J=7.6 Hz, 1H), 8.11 (d, J=8.8 Hz, 1H), 8.02 (m, 2H), 7.94 (d, J=8.0 Hz, 1H), 7.85 (m, 1H), 7.56 (m, 1H), 7.42 (m, 1H), 7.36 (d, J=8.8 Hz, 1H).

Compound 135: 1-(3-Phenyl-[1,2,4]oxadiazol-5-yl)-naphthalen-2-ol

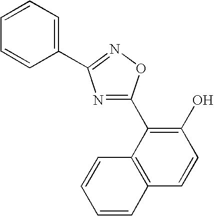

Benzoic acid and 2-hydroxy-1-naphthoic acid were subjected to protocol outlined in example 2 to produce the desired product. ¹H (400 MHz, d₆-DMSO) δ 10.64 (s, 1H), 8.78 (d, J=4.0 Hz, 1H), 8.19 (d, J=7.6 Hz, 1H), 8.02 (m, 2H), 7.62 (m, 1H), 7.54 (m, 1H), 7.13 (d, J=7.6 Hz, 1H), 7.05 (m, 1H).

1,3,4-Oxadiazoles

Scheme 9 shows a general procedure to prepare the 1,3,4-oxadiazoles.

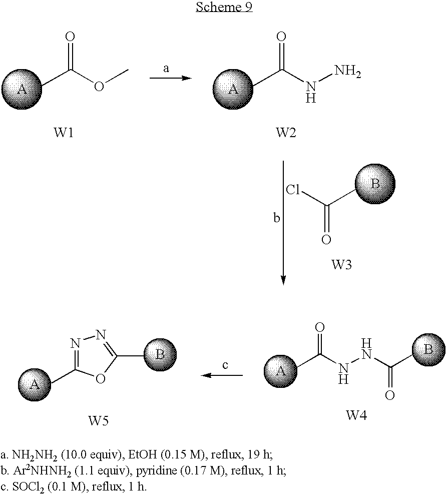

In an exemplary synthetic route, the methyl ester W1 was first treated with anhydrous hydrazine in ethanol and refluxed overnight to furnish the hydrazide W2. Reaction with various acyl chlorides W3, either commercially available or readily prepared from the corresponding carboxylic acids, in refluxing pyridine furnished the diacylhydrazides W4. The desired 1,3,4-oxadiazoles W5 were obtained after heating the diacylhydrazides in thionyl chloride at reflux. Every final product was purified by flash chromatography or preparative HPLC.

Compound 304: 1-(5-Phenyl-[1,3,4]oxadiazol-2-yl)-naphthalen-2-ol

### EXAMPLE 34

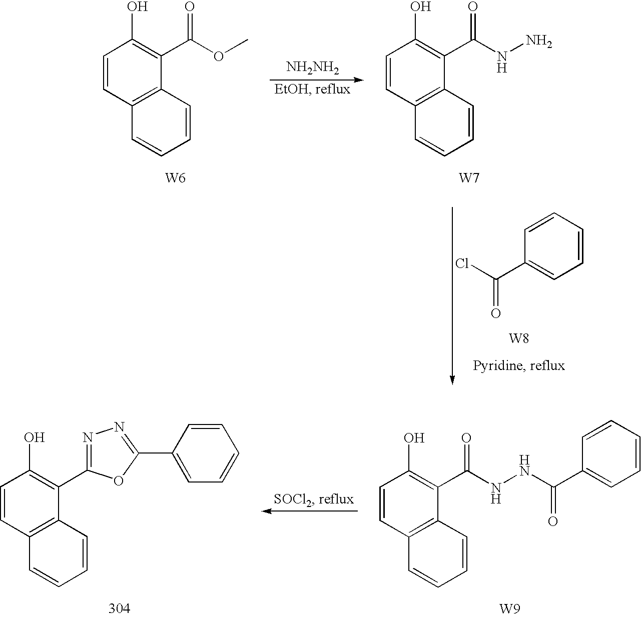

Step 1: Preparation of 2-Hydroxy-naphthalene-1-carboxylic acid hydrazide (W7)

To a solution of the methyl ester W6 (3.00 g, 14.8 mmol) in 100 ml of ethanol, anhydrous hydrazine (4.7 ml, 148 mmol, 10 equiv) was added and the solution was heated at 15 reflux for 19 h under inert atmosphere. The reaction mixture was cooled to room temperature and concentrated to dryness. Recrystallization of the residue from methanol-chloroform afforded 2.37 g (79%) of the acylhydrazide W7 as white needles; [M+H]⁺ 203.2; R_{f}: 0.70 (20% MeOH/CHCl₃); ¹H (400 MHz, DMSO) δ 9.38 (bs, 1H), 7.77-7.75 (m, 2H), 7.65 (d, J=8.4 Hz, 1H), 7.40 (ddd, J=8.0, 6.8, 0.8 Hz, 1H), 7.27 (ddd, J=7.6, 6.8, 1.2 Hz, 1H), 7.14 (d, J=9.2 Hz, 1H), 4.51 (bs, 2H).

Step 2: Preparation of Benzoic acid N′-(2-hvdroxy-naphthalene-1-carbonyl)-hydrazide (W9) A solution of the acid chloride W8 (0.128 ml, 1.1 mmol, 1.1 equiv) in 3 ml of pyridine was added to a solution of the hydrazide W7 (202 mg, 1.0 mmol) in 3 ml of pyridine, and the mixture was heated at reflux under inert atmosphere for 1 h. The solvent was removed under reduced pressure, and the residue was purified by flash chromatography (0→2.5% MeOH/CHCl₃) to afford 284 mg (93%) of the diacylhydrazide W9. [M+H]⁺ 307.2; R_{f}: 0.41 (5% MeOH/CHCl₃); ¹H (400 MHz, DMSO) δ 10.65 (s, 1H), 10.23 (s, 1H), 10.00 (s, 1H), 8.20 (d, J=8.4 Hz, 1H), 7.99-7.97 (m, 2H), 7.81 (dd, J=9.2, 8.4 Hz, 2H), 7.61-7.45 (m, 4H), 7.31 (ddd, J=8.0, 6.8, 0.8 Hz, 1H), 7.19 (d, J=8.8 Hz, 1H).

Step 3

Compound 304: Preparation of 1-(5-Phenyl-[1,3,4]oxadiazol-2-yl)-naphthalen-2-ol

The diacylhydrazide W9 (281 mg, 0.917 mmol) was dissolved in 10 ml of thionyl chloride and heated at reflux for 1 h under inert atmosphere. The mixture was concentrated to dryness, and the residue was purified by flash chromatography (0→2.5% MeOH/CHCl₃) to afford 222 mg (84%) of the oxadiazole 304 as a white solid. [M+H]⁺ 289.2; R_{f}: 0.94 (5% MeOH/CHCl₃); ¹H (400 MHz, DMSO) δ 10.81 (s, 1H), 8.09-8.04 (m, 3H), 7.91 (d, J=8.8 Hz, 2H), 7.65-7.59 (m, 3H), 7.53 (ddd, J=8.0, 6.8, 1.2 Hz, 1H), 7.39 (ddd, J=8.0, 6.8, 1.2 Hz, 1H), 7.33 (d, J=8.4 Hz, 1H).

The following compounds were prepared according to the procedure described in step 1.

2-Methyl-benzoic acid hydrazide (W10)

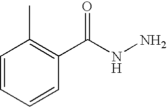

Purified by flash chromatography (0-5% MeOH/CHCl₃); [M+H]⁺ 151.1; R_{f}: 0.57 (10% MeOH/CHCl₃); ¹H (400 MHz, DMSO) δ 9.34 (s, 1H), 7.31-7.18 (m, 4H), 4.42 (s, 2H), 2.32 (s, 3H).

2-Hydroxy-benzoic acid hydrazide (W11)

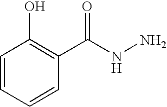

Recrystallized from MeOH/CHCl₃; [M+H]⁺ 153.2; R_{f}: 0.59 (10% MeOH/CHCl₃); 1H (400 MHz, DMSO) δ 12.42 (bs, 1H), 10.01 (bs, 1H), 7.77 (dd, J=8.0, 1.6 Hz, 1H), 7.35 (ddd, J=8.4, 6.8, 1.6 Hz, 1H), 6.87 (dd, J=8.4, 1.2 Hz, 1H), 6.83 (ddd, J=8.0, 7.2, 1.2 Hz, 1H), 4.62 (bs, 2H).

2-Methoxy-benzoic acid hydrazide (W12)

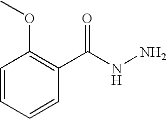

Purified by flash chromatography (0-5% MeOH/CHCl₃); [M+H]⁺ 167.2; R_{f}: 0.59 (10% MeOH/CHCl₃); ¹H (400 MHz, DMSO) δ 9.17 (s, 1H), 7.65 (dd, J=8.0, 1.6 Hz, 1H), 7.42 (ddd, J=10.0, 8.4, 2.0 Hz, 1H), 7.09 (dd, J=8.4, 0.8 Hz, 1H), 6.99 (ddd, J=7.6, 7.6, 0.8 Hz, 1H), 4.80 (s, 2H), 3.84 (s, 3H).

2-Fluoro-benzoic acid hydrazide (W13)

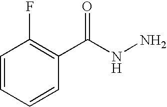

Purified by flash chromatography (0-5% MeOH/CHCl₃); [M+H]⁺ 155.1; R_{f}: 0.55 (10% MeOH/CHCl₃); ¹H (400 MHz, DMSO) δ 9.48 (s, 1H), 7.56-7.46 (m, 2H), 7.26-7.22 (m, 2H), 4.52 (s, 2H).

The following compounds were prepared according to the procedure described in step 2.

Fluoro-benzoic acid N′-(2-hydroxy-naphthalene-1-carbonl)-hydrazide (W14)

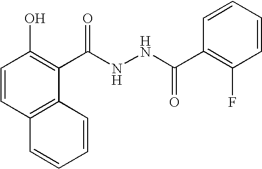

[M+H]⁺ 325.2; R_{f}: 0.31 (5% MeOH/CHCl₃); ¹H (400 MHz, DMSO) δ 10.43 (s, 1H), 10.30 (bs, 1H), 9.97 (bs, 1H), 8.15 (d, J=8.4 Hz, 1H), 7.83-7.79 (m, 2H), 7.71 (ddd, J=7.6, 7.2, 1.6 Hz, 1H), 7.61-7.55 (m, 1H), 7.46 (ddd, J=8.0, 6.4, 1.2 Hz, 1H), 7.35-7.29 (m, 3H), 7.18 (d, J=9.2 Hz, 1H).

2-Nitro-benzoic acid N′-(2-hydroxy-naphthalene-1-carbonyl)-hydrazide (W15)

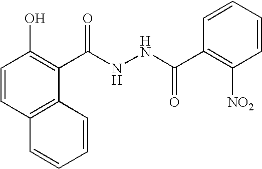

[M+H]⁺ 352.2; R_{f}0.15 (5% MeOH/CHCl₃); ¹H (400 MHz, DMSO) δ 10.78 (s, 1H), 10.44 (s, 1H), 10.00 (s, 1H), 8.08 (d, J=8.4 Hz, 1H), 7.91-7.73 (m, 5H), 7.45 (ddd, J=8.4, 6.8, 1.6 Hz, 1H), 7.30 (ddd, J=8.0, 6.8, 0.8 Hz, 1H), 7.19 (d, J=9.6 Hz, 1H).

2-Trifluoromethyl-benzoic acid N′-(2-hydroxy-naphthalene-1-carbonyl)-hydrazide (W16)

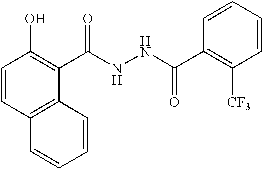

[M+H]⁺ 375.2; R_{f}: 0.26 (5% MeOH/CHCl₃); 1H (400 MHz, DMSO) δ 10.54 (s, 1H), 10.35 (s, 1H), 9.98 (s, 1H), 8.11 (d, J=8.0 Hz, 1H), 7.84-7.71 (m, 6H), 7.46 (ddd, J=8.4, 6.8, 1.2 Hz, 1H), 7.61 (ddd, J=8.4, 6.8, 1.2 Hz, 1H), 7.18 (d, J=9.2 Hz, 1H).

2,6-Dichloro-benzoic acid N′-(2-hydroxy-naiphthalene-1-carbonvl)-hydrazide (W17)

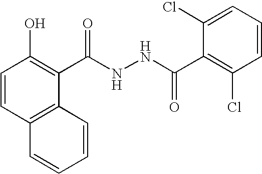

[M+H]⁺ 375.1; R_{f}: 0.26 (5% MeOH/CHCl₃); ¹H (400 MHz, DMSO) δ 10.74 (s, 1H), 10.38 (s, 1H), 9.98 (s, 1H), 8.08 (d, J=8.4 Hz, 1H), 7.82-7.79 (m, 2H), 7.54-7.44 (m, 4H), 7.31 (ddd, J=8.0, 7.2, 1.2 Hz, 1H), 7.18 (d, J=8.8 Hz, 1H).

3-Trifluoromethyl-pyridine-2-carboxylic acid N′-(2-hydroxy-naphthalene-1-carbonyl)-hydrazide (W18)

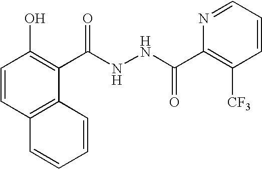

[M+H]⁺ 376.3; ¹H (400 MHz, DMSO) δ 10.72 (s, 1H), 10.38 (s, 1H), 10.00 (bs, 1H), 8.91 (dd, J=4.8, 0.8 Hz, 1H), 8.34 (dd, J=8.0, 1.6 Hz, 1H), 8.08 (d, J=8.4 Hz, 1H), 7.83-7.77 (m, 3H), 7.46 (ddd, J=8.0, 6.8, 1.2 Hz, 1H), 7.31 (ddd, J=7.6, 6.8, 0.8 Hz, 1H), 7.19 (d, J=8.8 Hz, 1H).

3-Chloro-5-trifluoromethyl-1yridine-2-carboxylic acid NA-(2-hydroxy-naphthalene-1-carbonyl)-hydrazide (W19)

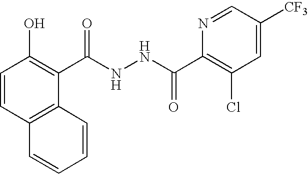

[M+H]⁺ 410.2; R_{f}: 0.32 (5% MeOH/CH₂Cl₂); ¹H (400 MHz, DMSO) δ 10.82 (s, 1H), 10.46 (bs, 1H), 9.99 (bs, 1H), 9.03 (s, 1H), 8.64 (d, J=1.6 Hz, 1H), 8.09 (d, J=8.4 Hz, 1H), 7.83-7.79 (m, 2H), 7.46 (ddd, J=8.0, 6.7, 1.2 Hz, 1H), 7.31 (ddd, J=7.6, 6.4, 0.8 Hz, 1H), 7.18 (d, J=9.2 Hz, 1H).

Furan-2-carboxylic acid N′-(2-hydroxy-naphthalene-1-carbonyl)-hydrazide (W20)

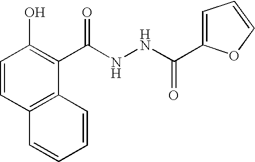

[M+H]⁺ 297.2; R_{f}: 0.46 (5% MeOH/CH₂Cl₂); ¹H (400 MHz, DMSO) δ 10.52 (s, 1H), 10.19 (bs, 1H), 9.97 (bs, 1H), 8.15 (d, J=8.4 Hz, 1H), 7.91 (dd, J=2.0, 1.2 Hz, 1H), 7.83-7.78 (m, 2H), 7.45 (ddd, J=8.4, 6.8, 1.6 Hz, 1H), 7.32-7.28 (m, 2H), 7.18 (d, J=8.8 Hz, 1H), 6.67 (dd, J=4.4, 2.0 Hz, 1H).

Thiophene-2-carboxylic acid N′-(2-hydroxy-naphthalene-1-carbonyl)-hydrazide (W21)

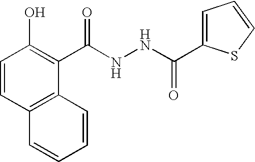

[M+H]⁺ 313.1; R_{f}: 0.41 (5% MeOH/CH₂Cl₂); ¹H (400 MHz, DMSO) δ 10.66 (s, 1H), 10.23 (s, 1H), 10.00 (s, 1H), 8.16 (d, J=8.4 Hz, 1H), 7.94 (dd, J=3.6, 1.2 Hz, 1H), 7.86 (dd, J=4.4, 1.2 Hz, 1H), 7.83-7.79 (m, 2H), 7.46 (ddd, J=8.4, 6.8, 1.2 Hz, 1H), 7.31 (ddd, J=7.6, 6.4, 0.8 Hz, 1H), 7.22-7.17 (m, 2H).

Benzoic acid N′-(2-methyl-benzoyl)-hvdrazide (W22)

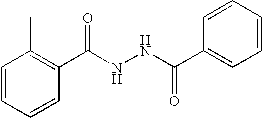

[M+H]⁺ 254.9; R_{f}: 0.39 (5% MeOH/CHCl₃); ¹H (400 MHz, DMSO) δ 10.47 (s, 1H), 10.13 (s, 1H), 7.93-7.91 (m, 2H), 7.59-7.55 (m, 1H), 7.52-7.48 (m, 2H), 7.43 (d, J=7.6 Hz, 1H), 7.89-7.35 (m, 1H), 7.29-7.25 (m, 2H), 2.43 (s, 3H).

2-Fluoro-benzoic acid N′-(2-methyl-benzovl)-hydrazide (W23)

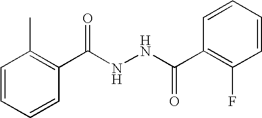

[M+H]⁺ 273.0; R_{f}: 0.53 (5% MeOH/CHCl₃); ¹H (400 MHz, DMSO) δ 10.26 (bs, 2H), 7.67 (ddd, J=7.2, 7.2, 1.6 Hz, 1H), 7.60-7.55 (m, 1H), 7.43-7.24 (m, 6H), 2.43 (s, 3H).

2-Nitro-benzoic acid N′-(2-methyl-benzoyl)-hydrazide (W24)

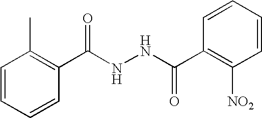

[M+H]⁺ 299.9; R_{f}: 0.19 (5% MeOH/CHCl₃); ¹H (400 MHz, DMSO) δ 10.65 (s, 1H), 10.38 (s, 1H), 8.06 (d, J=8.8 Hz, 1H), 7.85 (ddd, J=8.8, 7.2, 1.2 Hz, 1H), 7.77-7.73 (m, 2H), 7.42-7.35 (m, 2H), 7.29-7.18 (m, 2H), 2.41 (s, 3H).

2-Trifluoromethyl-benzoic acid N′-(2-methyl-benzoyl)-hydrazide (W25)

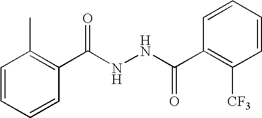

[M+H]⁺ 322.9; R_{f}: 0.28 (5% MeOH/CHCl₃); ¹H (400 MHz, DMSO) δ 10.41 (s, 1H), 10.28 (s, 1H), 7.83-7.81 (m, 2H), 7.71-7.68 (m, 2H), 7.42 (dd, J=7.6, 1.2 Hz, 1H), 7.37 (ddd, J=7.6, 7.6, 1.6 Hz, 1H), 7.28-7.24 (m, 2H), 2.42 (s, 3H).

2,6-Dichloro-benzoic acid N′-(2-methyl-benzoyl)-hydrazide (W26)

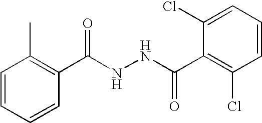

[M+H]⁺ 322.9; R_{f}: 0.25 (5% MeOH/CHCl₃); ¹H (400 MHz, DMSO) δ 10.64 (s, 1H), 10.35 (s, 1H), 7.53-7.34 (m, 5H), 7.28-7.23 (m, 2H), 2.41 (s, 3H).

Pyridine-2-carboxylic acid N′-(2-methyl-benzoyl)-hydrazide (W27)

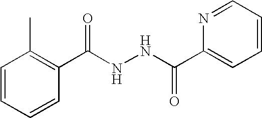

[M+H]⁺ 256.2.

3-Trifluoromethyl-pyridine-2-carboxylic acid N′-(2-methyl-benzoyl)-hydrazide (W28):

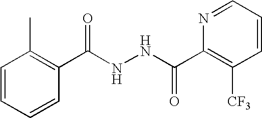

[M+H]⁺ 324.1.

5-Chloro-3-trifluoromethyl-pyridine-2-carboxylic acid N′-(2-methyl-benzoyl)-hydrazide (W29)

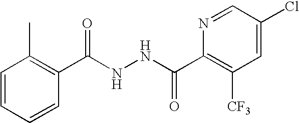

[M+H]⁺ 358.2.

Furan-2-carboxylic acid N′-(2-methyl-benzoyl)-hvdrazide (W30)

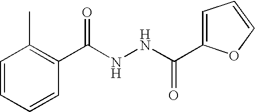

[M+H]⁺ 244.9; R_{f}: 0.19 (5% MeOH/CHCl₃); ¹H (400 MHz, DMSO) δ 10.35 (s, 1H), 10.10 (s, 1H), 7.90 (d, J=1.6 Hz, 1H), 7.40-7.34 (m, 2H), 7.28-7.24 (m, 3H), 6.66 (dd, J=3.6, 1.6 Hz, 1H), 2.41 (s, 3H).

Thiophene-2-carboxylic acid N′-(2-methyl-benzoyl)-hydrazide (W31)

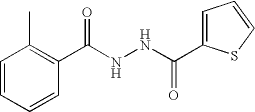

[M+H]⁺ 260.9; R_{f}: 0.16 (5% MeOH/CHCl₃); ¹H (400 MHz, DMSO) δ 10.50 (s, 1H), 10.16 (s, 1H), 7.88 (dd, J=7.6, 0.8 Hz, 1H), 7.84 (dd, J=5.2, 1.2 Hz, 1H), 7.42-7.35 (m, 2H), 7.28-7.25 (m, 2H), 7.19 (dd, J=5.6, 4.0 Hz, 1H), 2.42 (s, 3H).

Benzoic acid N′-(2-hydroxy-benzoyl)-hydrazide (W32)

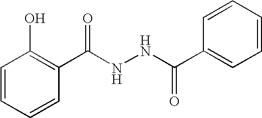

[M+H]⁺ 257.1; ¹H (400 MHz, DMSO) δ 11.92 (s, 1H), 10.66 (d, J=5.6 Hz, 2H), 7.95-7.91 (m, 3H), 7.60-7.43 (m, 4H), 6.98-6.92 (m, 2H).

2-Fluoro-benzoic acid N′-(2-hydroxy-benzoyl)-hydrazide (W33)

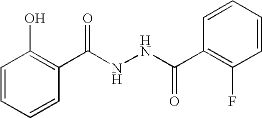

[M+H]⁺ 275.2; ¹H (400 MHz, DMSO) δ 11.83 (s, 1H), 10.66 (s, 1H), 10.44 (s, 1H), 7.84 (dd, J=8.0, 1.6 Hz, 1H), 7.60 (ddd, J=7.6, 7.2, 1.6 Hz, 1H), 7.52-7.48 (m, 1H), 7.37 (ddd, J=8.4, 7.2, 1.6 Hz, 1H), 7.28-7.23 (m, 2H), 6.90-6.84 (m, 2H).

2-Nitro-benzoic acid N′-(2-hydroxy-benzoyl)-hydrazide (W34)

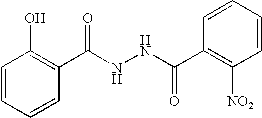

[M+H]⁺ 302.3; ¹H (400 MHz, DMSO) δ 11.88 (s, 1H), 10.96 (d, J=2.0 Hz, 1H), 10.83 (d, J=1.6 Hz, 1H), 8.09 (dd, J=7.6, 2.0 Hz, 1H), 7.95 (dd, J=7.6, 1.6 Hz, 1H), 7.86 (dd, J=8.4, 8.0, 1.2 Hz, 1H), 7.78-7.73 (m, 2H), 7.45 (ddd, J=8.8, 7.6, 1.6 Hz, 1H), 6.99-6.92 (m, 2H).

2-Trifluoromethyl-benzoic acid N′-(2-hydroxy-benzoyl)-hydrazide (W35)

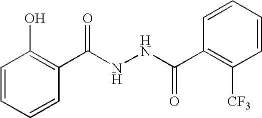

[M+H]⁺ 325.3; ¹H (400 MHz, DMSO) δ 11.93 (s, 1H), 10.76 (d, J=1.6 Hz, 1H), 10.69 (d, J=1.6 Hz, 1H), 7.95 (dd, J=8.0, 1.6 Hz, 1H), 7.84-7.77 (m, 2H), 7.73-7.69 (m, 2H), 7.45 (ddd, J=8.8, 7.6, 1.6 Hz, 1H), 6.98 (dd, J=8.4, 1.2 Hz, 1H), 6.94 (dd, J=8.0, 1.2 Hz, 1H).

2,6-Dichloro-benzoic acid N′-(2-hydroxy-benzoyl)-hydrazide (W36)

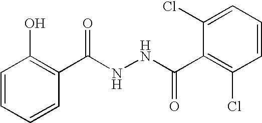

[M+H]⁺ 325.2; ¹H (400 MHz, DMSO) δ 11.85 (s, 1H), 11.02 (d, J=2.4 Hz, 1H), 10.77 (d, J=3.2 Hz, 1H), 7.88 (dd, J=7.6, 1.6 Hz, 1H), 7.47-7.35 (m, 4H), 6.92-6.85 (m, 2H).

Pyridine-2-carboxylic acid N′-(2-hydroxy-benzoyl)-hydrazide (W37)

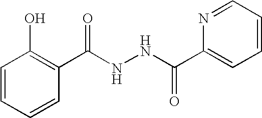

[M+H]⁺ 258.1.

3-Trifluoromethyl-pyridine-2-carboxylic acid N′-(2-hydroxy-benzoyl)-hydrazide (W38)

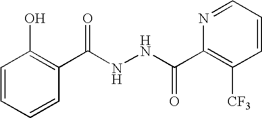

[M+H]⁺ 326.2.

3-Chloro-5-trifluoromethyl-pyridine-2-carboxylic acid N′-(2-hvdroxy-benzoyl)-hvdrazide (W39)

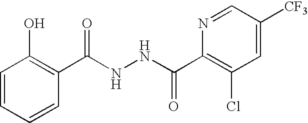

[M+H]⁺ 360.2.

Furan-2-carboxylic acid N′-(2-hydroxy-benzoyl)-hydrazide (W40)

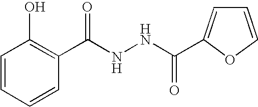

[M+H]⁺ 247.2; ¹H (400 MHz, DMSO) δ 11.79 (s, 1H), 10.50 (s, 1H), 10.44 (s, 1H), 7.83-7.81 (m, 2H), 7.37 (ddd, J=9.2, 7.2, 2.0 Hz, 1H), 7.19 (dd, J=3.2, 0.8 Hz, 1H), 6.90-6.84 (m, 2H), 6.59 (dd, J=3.6, 1.6 Hz, 1H).

Thiophene-2-carboxylic acid N′-(2-hydroxy-benzoyl)-hydrazide (W41)

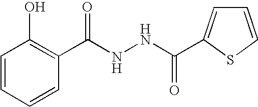

[M+H]⁺ 263.1; ¹H (400 MHz, DMSO) δ 11.76 (s, 1H), 10.58 (s, 1H), 10.55 (s, 1H), 7.83-7.81 (m, 2H), 7.77 (d, J=5.2, 0.8 Hz, 1H), 7.37 (ddd, J=8.4, 8.4, 1.6 Hz, 1H), 7.13 (dd, J=5.2, 3.6 Hz, 1H), 6.90-6.85 (m, 2H).

Benzoic acid N′-(2-methoxy-benzoyl)-hydrazide (W42)

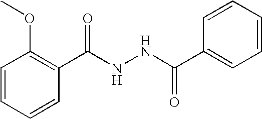

[M+H]⁺ 271.2; ¹H (400 MHz, DMSO) δ 10.62 (d, J=1.6 Hz, 1H), 10.03 (d, J=2.0 Hz, 1H), 7.94-7.92 (m, 2H), 7.78 (dd, J=7.2, 1.6 Hz, 1H), 7.89-7.47 (m, 4H), 7.16 (d, J=8.0 Hz, 1H), 7.07 (ddd, J=8.0, 7.2, 1.2 Hz, 1H), 3.91 (s, 3H).

2-Fluoro-benzoic acid N′-(2-methoxy-benzoyl)-hydrazide (W43)

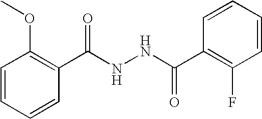

[M+H]⁺ 272.1.

3-Trifluoromethyl-piridine-2-carboxylic acid N′-(2-methoxy-benzoyl)-hydrazide (W48)

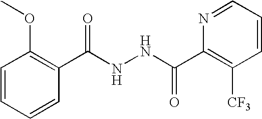

[M+H]⁺ 340.2.

5-Chloro-3-trifluoromethyl-pyridine-2-carboxylic acid N′-(2-methoxy-benzoyl)-hvdrazide (W49)

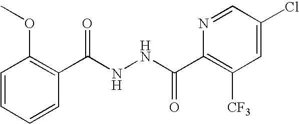

[M+H]⁺ 374.3.

Furan-2-carboxylic acid N′-(2-methoxy-benzoyl)-hydrazide (W50)

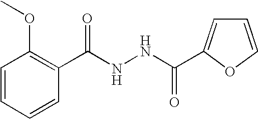

[M+H]⁺ 261.1; ¹H (400 MHz, DMSO) δ 10.44 (d, J=1.2 Hz, 1H), 9.92 (d, J=1.2 Hz, 1H), 7.87 (dd, J=2.0, 0.8 Hz, 1H), 7.73 (dd, J=8.0, 2.4 Hz, 1H), 7.50 (ddd, J=8.4, 7.6, 2.0 Hz, 1H), 7.25 (dd, J=3.2, 0.8 Hz, 1H), 7.15 (d, J=7.6 Hz, 1H), 7.05 (ddd, J=7.6, 7.6, 0.8 Hz, 1H), 6.65 (dd, J=3.2, 1.6 Hz, 1H), 3.89 (s, 3H).

Thiolphene-2-carboxylic acid N′-(2-methoxy-benzoyl)-hydrazide (W51)

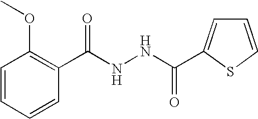

[M+H]⁺ 277.1; ¹H (400 MHz, DMSO) δ 10.62 (d, J=1.2 Hz, 1H), 10.00 (d, J=0.8 Hz, 1H), 7.90 (dd, J=3.6, 1.2 Hz, 1H), 7.82 (dd, J=5.2, 1.2 Hz, 1H), 7.73 (dd, J=7.6, 1.6 Hz, 1H), 7.50 (ddd, J=9.2, 8.0, 2.0 Hz, 1H), 7.18 (dd, J=4.4, 3.2 Hz, 1H), 7.15 (d, J=7.6 Hz, 1H), 7.05 (ddd, J=8.0, 7.6, 0.8 Hz, 1H), 3.89 (s, 3H).

Benzoic acid N′-(2-fluoro-benzoyl)-hydrazide (W52)

[M+H]⁺ 289.1; ¹H (400 MHz, DMSO) δ 10.46 (s, 1H), 10.08 (s, 1H), 7.78 (dd, J=7.6, 1.6 Hz, 1H), 7.67 (ddd, J=7.2, 7.2, 2.0 Hz, 1H), 7.59-7.49 (m, 2H), 7.33-7.29 (m, 2H), 7.16 (d, J=8.4 Hz, 1H), 7.06 (ddd, J=8.0, 7.6, 0.8 Hz, 1H), 3.91 (s, 3H).

2-Nitro-benzoic acid N′-(2-methoxy-benzoyl)-hydrazide (W44)

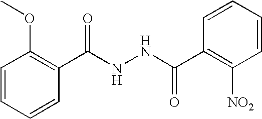

[M+H]⁺ 316.1; ¹H (400 MHz, DMSO) δ 10.92 (d, J=2.4Hz, 1H), 10.19 (d, J=2.0 Hz, 1H), 8.06 (dd,J=8.0,7.2 Hz, 1H), 7.84 (ddd,J=8.4,6.8, 1.2 Hz, 1H), 7.76-7.72 (m, 3H), 7.51 (ddd, J=8.4, 7.6, 2.0 Hz, 1H), 7.17 (dd, J=8.0, 0.8 Hz, 1H), 7.06 (ddd, J=7.6, 7.2, 0.8 Hz, 1H), 3.91 (s, 3H).

2-Trifluoromethyl-benzoic acid N′-(2-methoxy-benzoyl)-hydrazide (W45)

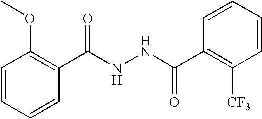

[M+H]⁺ 339.3; ¹H (400 MHz, DMSO) δ 10.63 (d, J=2.4 Hz, 1H), 10.08 (d, J=2.0 Hz, 1H), 7.83-7.75 (m, 3H), 7.72-7.70 (m, 2H), 7.51 (ddd, J=8.8, 7.2, 2.0 Hz, 1H), 7.17 (d, J=8.0 Hz, 1H), 7.07 (ddd, J=7.6, 7.2, 0.8 Hz, 1H), 3.91 (s, 3H).

2,6-Dichloro-benzoic acid N′-(2-methoxy-benzoyl)-hydrazide (W46)

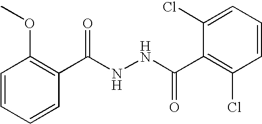

[M+H]⁺ 339.2; ¹H (400 MHz, DMSO) δ 11.11 (d,J=3.6 Hz, 1H), 10.25 (d,J=3.2 Hz, 1H), 7.76 (dd, J=7.6, 2.0 Hz, 1H), 7.52-7.43 (m, 4H), 7.17 (d, J=8.0 Hz, 1H), 7.07 (ddd, J=7.6, 7.2, 0.8 Hz, 1H), 3.92 (s, 3H).

Pyridine-2-carboxylic acid N′-(2-methoxy-benzoyl)-hydrazide (W47)

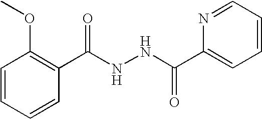

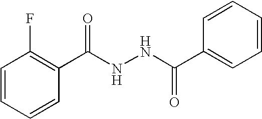

[M+H]⁺ 259.1; ¹H (400 MHz, DMSO) δ 10.60 (s, 1H), 10.32 (s, 1H), 7.93-7.91 (m, 2H), 7.67 (ddd, J=7.2, 6.8, 1.2 Hz, 1H), 7.60-7.48 (m, 4H), 7.37-7.30 (m, 2H).

2-Fluoro-benzoic acid N′-(2-fluoro-benzoyl)-hydrazide (W53)

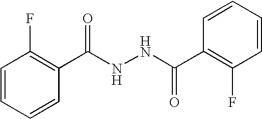

[M+H]⁺ 277.2; ¹H (400 MHz, DMSO) δ 10.40 (s, 2H), 7.66 (ddd, J=7.6, 7.2, 2.0 Hz, 2H), 7.60-7.54 (m, 2H), 7.34-7.29 (m, 4H).

2-Nitro-benzoic acid N′-(2-fluoro-benzoyl)-hydrazide (W54)

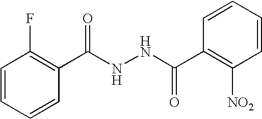

[M+H]⁺ 304.4; ¹H (400 MHz, DMSO) δ 10.76 (s, 1H), 10.56 (s, 1H), 8.08 (ddd, J=7.6, 2.0, 0.8 Hz, 1H), 7.87-7.83 (m, 1H), 7.78-7.71 (m, 2H), 7.68-7.62 (m, 1H), 7.60-7.54 (m, 1H), 7.34-7.29 (m, 2H).

2-Trifluoromethyl-benzoic acid N′-(2-fluoro-benzoyl)-hydrazide (W55)

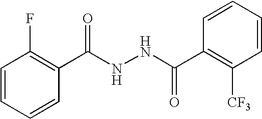

[M+H]⁺ 327.2; ¹H (400 MHz, DMSO) δ 10.53 (s, 1H), 10.46 (s, 1H), 7.83-7.77 (m, 2H), 7.72-7.64 (m, 3H), 7.60-7.54 (m, 1H), 7.34-7.29 (m, 2H).

2,6-Dichloro-benzoic acid N′-(2-fluoro-benzoyl)-hydrazide (W56)

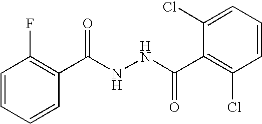

[M+H]⁺ 327.2; ¹H (400 MHz, DMSO) δ 10.78 (s, 1H), 10.56 (s, 1H), 7.64 (ddd, J=7.6, 7.2, 1.6 Hz, 1H), 7.59-7.44 (m, 4H), 7.33-7.28 (m, 2H).

Pyridine-2-carboxylic acid N′-(2-fluoro-benzoyl)-hydrazide (W57)

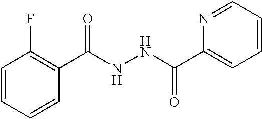

[M+H]⁺ 260.2.

3-Trifluoromethyl-pyridine-2-carboxylic acid N′-(2-fluoro-benzoyl)-hydrazide (W58)

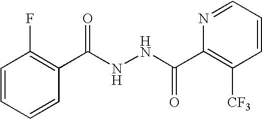

[M+H]⁺ 328.1.

5-Chloro-3-trifluoromethyl-pyridine-2-carboxylic acid N′-(2-fluoro-benzoyl)-hydrazide (W59)

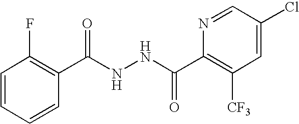

[M+H]⁺ 362.2.

Furan-2-carboxylic acid N′-(2-fluoro-benzoyl)-hydrazide (W60)

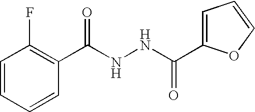

[M+H]⁺ 249.2; R_{f}: XXX (5% MeOH/CHCl₃); ¹H (400 MHz, DMSO) δ 10.45 (s, 1H), 10.27 (s, 1H), 7.89 (d, J=1.2 Hz, 1H), 7.64 (ddd, J=7.2, 7.2, 1.6 Hz, 1H), 7.60-7.54 (m, 1H), 7.34-7.25 (m, 3H), 6.66 (dd, J=2.8, 1.2 Hz, 1H).

Thiophene-2-carboxylic acid N′-(2-fluoro-benzoyl)-hydrazide (W61)

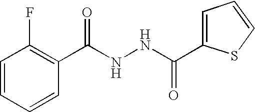

[M+H]⁺ 265.2; ¹H (400 MHz, DMSO) δ 10.59 (s, 1H), 10.33 (s, 1H), 7.88 (dd, J=3.6, 0.8 Hz, 1H), 7.83 (dd, J=5.2, 1.2 Hz, 1H), 7.64 (ddd, J=7.6, 7.6, 2.0 Hz, 1H), 7.60-7.54 (m, 1H), 7.34-7.30 (m, 2H), 7.19 (dd, J=4.8, 3.6 Hz, 1H).

Furan-2-carboxylic acid N′-(2-hvdroxy-5-methoxy-benzoyl)-hydrazide (W62)

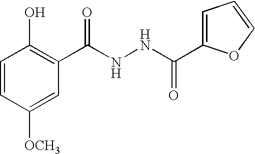

[M+H]⁺ 277.2.

Thiophene-2-carboxylic acid N′-(2-hydroxy-5-methoxy-benzoyl)-hydrazide (W63)

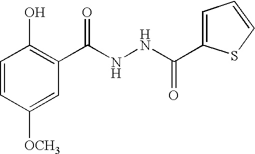

[M+H]⁺ 293.1.

Pyridine-2-carboxylic acid N′-(2-hydroxy-5-methoxy-benzoyl)-hydrazide (W64)

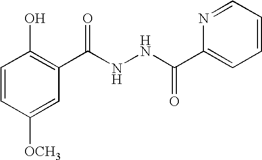

[M+H]⁺ 288.2.

3-Trifluoromethyl-pyridine-2-carboxylic acid N′-(2-hydroxy-5-methoxy-benzoyl)-hydrazide (W65)

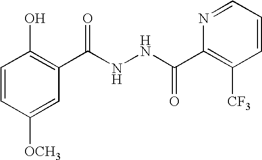

[M+H]⁺ 356.2.

3-Chloro-5-trifluoromethyl-pyridine-2-carboxylic acid N′-(2-hydroxy-5-methoxy-benzoyl)-hydrazide (W66)

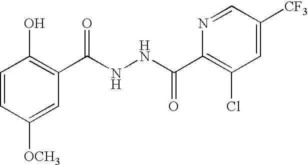

[M+H]⁺ 390.2.

The following compound were prepared according to the procedures in step 3.

Compound 305: 1-[5-(2-Fluoro-phenyl)-[1,3,4]oxadiazol-2-yl]-naphthalen-2-ol

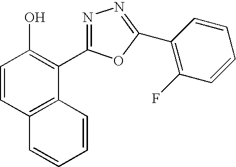

[M+H]⁺ 307.2; R_{f}: 0.95 (5% MeOH/CHCl₃); ¹H (400 MHz, DMSO) δ 10.82 (s, 1H), 8.11 (ddd, J=7.6, 7.2, 1.6 Hz, 1H), 8.06 (d, J=8.8 Hz, 1H), 7.93-7.91 (m, 2H), 7.73-7.68 (m, 1H), 7.55-7.43 (m, 3H), 7.39 (ddd, J=8.4, 7.2, 1.2 Hz, 1H), 7.32 (d, J=8.4 Hz, 1H).

Compound 311: 1-[5-(2-Nitro-phenyl)-[1,3,4]oxadiazol-2-yl]-naphthalen-2-ol

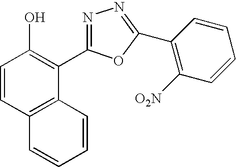

[M+H]⁺ 334.1; R_{f}: 0.78 (5% MeOH/CH₂Cl₂); ¹H (400 MHz, DMSO) δ 10.81 (s, 1H), 8.20 (dd, J=7.6, 1.6 Hz, 1H), 8.15 (dd, J=7.6, 1.6 Hz, 1H), 8.06 (d, J=8.8 Hz, 1H), 7.98-7.91 (m, 2H), 7.86 (d, J=8.4 Hz, 1H), 7.53 (ddd, J=8.4, 6.8, 1.6 Hz, 1H), 7.40 (ddd, J=8.0, 6.8, 0.8 Hz, 1H), 7.31 (d, J=8.4 Hz, 1H).

Compound 306: 1-[5-(2-Trifluoromethyl-phenyl)-[1,3,4]oxadiazol-2-yl]-naphthalen-2-ol

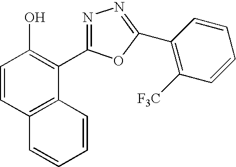

[M+H]⁺ 357.2; R_{f}: 0.94 (5% MeOH/CH₂Cl₂); ¹H (400 MHz, DMSO) δ 10.81 (s, 1H), 8.15 (d, J=7.2 Hz, 1H), 8.06 (d, J=9.6 Hz, 1H), 8.03 (dd, J=7.6, 1.2 Hz, 1H), 7.95-7.83 (m, 4H), 7.53 (ddd,J=8.0,6.8, 1.2 Hz, 1H), 7.39 (ddd,J=8.0,6.8, 1.2 Hz, 1H), 7.32 (d,J=8.4 Hz, 1H).

Compound 310: 1-[5-(2,6-Dichloro-phenyl)-[1,3,4]oxadiazol-2-yl]-naphthalen-2-ol

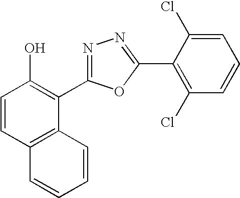

[M+H]⁺ 357.1; R_{f}: 0.97 (5% MeOH/CHCl₃); ¹H (400 MHz, DMSO) δ 10.84 (s, 1H), 8.06 (d, J=8.4 Hz, 1H), 7.32 (d,J=8.0 Hz, 1H), 7.80-7.73 (m, 3H), 7.55 (ddd,J=8.0, 6.4, 0.8 Hz, 1H), 7.40 (ddd,J=8.0, 6.8, 0.8 Hz, 1H), 7.32 (d, J=8.8 Hz, 1H).

Compound 338: 1-[5-(3-Trifluoromethyl-pyridin-2-yl)-[1,3,4]oxadiazol-2-yl]-naphthalen-2-ol

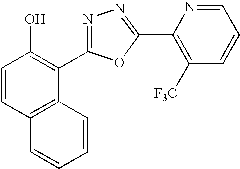

[M+H]⁺ 358.1; ¹H (400 MHz, DMSO) δ 10.88 (bs, 1H), 9.09 (d, J=5.2 Hz, 1H), 8.53 (dd, J=8.0, 0.8 Hz, 1H), 8.07 (d,J=8.8 Hz, 1H), 7.95-7.92 (m, 2H), 7.78 (dd, J=8.4, 1.2 Hz, 1H), 7.54 (ddd, J=8.4, 7.2, 1.6 Hz, 1H), 7.40 (ddd, J=8.0, 6.8, 1.6 Hz, 1H), 7.34 (d, J=9.2 Hz, 1H).

Compound 309: 1-[5-(3-Chloro-5-trifluoromethyl-pyridin-2-yl)-[1,3,4]oxadiazol-2-yl]-naphthalen-2-ol

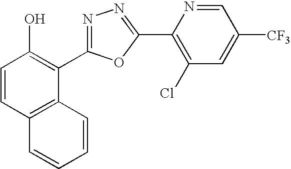

[M+H]⁺ 392.1; R_{f}: 0.92 (5% MeOH/CHCl₃); ¹H (400 MHz, DMSO) δ 10.87 (s, 1H), 9.17 (s, 1H), 8.80 (s, 1H), 8.07 (d, J=8.4 Hz, 1H), 7.92 (d, J=8.0 Hz, 1H), 7.90 (d, J=8.8 Hz, 1H), 7.53 (ddd, J=8.4, 6.8, 1.2 Hz, 1H), 7.40 (ddd, J=8.0, 6.8, 1.2 Hz, 1H), 7.33 (d, J=9.6 Hz, 1H).

Compound 307: 1-(5-Furan-2-yl-[1,3,4]oxadiazol-2-yl)-naphthalen-2-ol

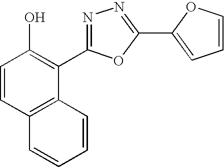

[M+H]⁺ 279.1; R_{f}: 0.92 (5% MeOH/CH₂Cl₂); ¹H (400 MHz, DMSO) δ 10.78 (s, 1H), 8.07 (dd, J=2.0, 0.8 Hz, 1H), 8.05 (d, J=8.8 Hz, 1H), 7.91 (d, J=8.4 Hz, 1H), 7.83 (dd, J=8.4, 0.4 Hz, 1H), 7.51 (ddd, J=8.4, 7.2, 2.0 Hz, 1H), 7.41-7.37 (m, 2H), 7.31 (d, J=9.6 Hz, 1H), 6.81 (dd, J=3.6, 2.0 Hz, 1H).

Compound 308: 1-(5-Thiophen-2-yl-[1,3,4]oxadiazol-2-yl)-naphthalen-2-ol

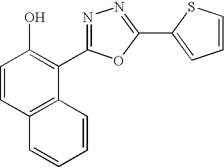

[M+H]⁺ 295.1; R_{f}: 0.93 (5% MeOH/CH₂Cl₂); ¹H (400 MHz, DMSO) δ 10.79 (s, 1H), 8.05 (d, J=8.4 Hz, 1H), 7.96 (dd, J=5.2, 1.6 Hz, 1H), 7.91 (d, J=8.0 Hz, 1H), 7.88-7.84 (m, 2H), 7.81 (ddd, J=8.4, 6.8, 1.2 Hz, 1H), 7.39 (ddd, J=8.0, 6.8, 0.8 Hz, 1H), 7.32-7.29 (m, 2H).

Compound 312: 2-Phenyl-5-o-tolyl-[1,3,4]oxadiazole

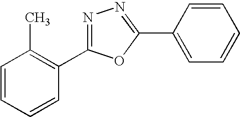

[M+H]⁺ 237.2; ¹H (400 MHz, CDCl₃) δ 8.13-8.11 (m, 2H), 8.02 (dd, J=8.4, 1.6 Hz, 1H), 7.54-7.50 (m, 3H), 7.43-7.31 (m, 3H), 2.77 (s, 3H).

Compound 313: 2-(2-Fluoro-phenyl)-5-o-tolyl-[1,3,4]oxadiazole

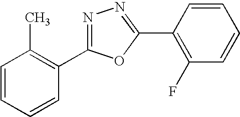

[M+H]⁺ 255.2; ¹H (400 MHz, CDCl₃) δ 8.14 (ddd, J=7.6, 7.2, 2.0 Hz, 1H), 8.04 (dd, J=8.0, 1.6 Hz, 1H), 7.56-7.50 (m, 1H), 7.44-7.39 (m, 1H), 7.35-7.23 (m, 4H), 2.77 (s, 3H).

Compound 315: 2-(2-Nitro-phenyl)-5-o-tolyl-[1,3,4]oxadiazole

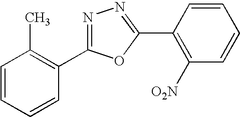

[M+H]⁺ 282.1; ¹H (400 MHz, CDCl₃) δ 8.01 (dd, J=7.6, 1.6 Hz, 1H), 7.92 (dd, J=7.6, 1.2 Hz, 1H), 7.84 (dd, J=8.0, 1.2 Hz, 1H), 7.69 (ddd, J=7.6, 7.6, 1.2 Hz, 1H), 7.65 (ddd, J=7.6, 7.6, 1.6 Hz, 1H), 7.34 (ddd, J=8.0, 7.6, 1.6 Hz, 1H), 7.27-7.21 (m, 2H), 2.63 (s, 3H).

Compound 314: 2-o-Tolyl-5-(2-trifluoromethyl-phenyl)-[1,3,4]oxadiazole

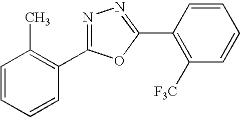

[M+H]⁺ 305.2; ¹H (400 MHz, CDCl₃) δ 8.18 (dd, J=8.0, 1.2 Hz, 1H), 8.01 (dd, J=8.0, 1.2 Hz, 1H), 7.88 (dd, J=7.6, 1.6 Hz, 1H), 7.75-7.67 (m, 2H), 7.45-7.32 (m, 3H), 2.78 (s, 3H).

Compound 316: 2-(2,6-Dichloro-phenyl)-5-o-tolyl-[1,3,4]oxadiazole

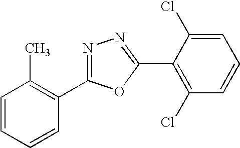

[M+H]⁺ 305.1; ¹H (400 MHz, CDCl₃) δ 8.01 (dd, J=8.0, 1.6 Hz, 1H), 7.49-7.41 (m, 4H), 7.37-7.31 (m, 2H), 2.77 (s, 3H).

Compound 339: 2-(5-o-Tolyl-[1,3,4]oxadiazol-2-yl)-pyridine

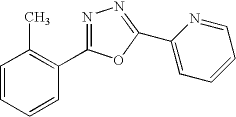

[M+H]⁺ 238.1; ¹H (400 MHz, DMSO) δ 8.79 (dd, J=4.8, 0.8 Hz, 1H), 8.24 (dd, J=8.0, 0.8 Hz, 1H), 8.06 (ddd, J=7.6, 7.6, 1.6 Hz, 1H), 8.01 (dd, J=7.6, 1.2 Hz, 1H), 7.64 (ddd, J=7.6, 4.8, 1.2 Hz, 1H), 7.54-7.41 (m, 3H), 2.69 (s, 3H).

Compound 340: 2-(5-o-Tolyl-[1,3,4]oxadiazol-2-yl)-3-trifluoromethyl-pyridine

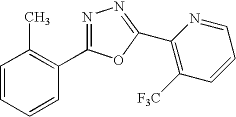

[M+H]⁺ 306.1; ¹H (400 MHz, DMSO) δ 9.09 (dd, J=4.4, 1.2 Hz, 1H), 8.51 (dd, J=4.4, 1.2 Hz, 1H), 7.96-7.91 (m, 2H), 7.54 (ddd, J=7.6, 7.2, 1.2 Hz, 1H), 7.48-7.41 (m, 2H), 2.68 (s, 3H).

Compound 341: 3-Chloro-2-(5-o-tolyl-[1,3,4]oxadiazol-2-yl)-5-trifluoromethyl-pyridine

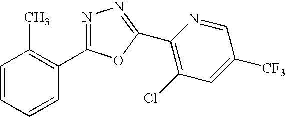

[M+H]⁺ 340.2; ¹H (400 MHz, DMSO) δ 9.18 (d, J=2.0 Hz, 1H), 8.78 (d, J=1.2 Hz, 1H), 7.99 (dd, J=3.6, 1.2 Hz, 1H), 7.54 (ddd, J=7.6, 7.6, 1.2 Hz, 1H), 7.48-7.42 (m, 2H), 2.70 (s, 3H).

Compound 303: 2-Furan-2-yl-5-o-tolyl-[1,3,4]oxadiazole

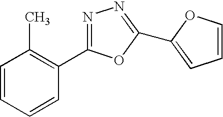

[M+H]⁺ 227.1; ¹H (400 MHz, CDCl₃) δ 7.99 (dd, J=7.2, 1.2 Hz, 1H), 7.65 (dd, J=1.6, 0.8 Hz, 1H), 7.41 (ddd, J=8.8, 7.2, 1.6 Hz, 1H), 7.35-7.30 (m, 2H), 7.21 (dd, J=3.6, 1.2 Hz 1H), 6.61 (dd, J=3.2, 1.2 Hz, 1H), 2.75 (s, 3H).

Compound 317: 2-Thiophen-2-yl-5-o-tolyl-[1,3,4]oxadiazole

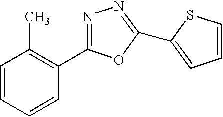

[M+H]⁺ 243.1; ¹H (400 MHz, CDCl₃) δ 7.98 (d, J=7.6 Hz, 1H), 7.81 (dd, J=3.6, 1.2 Hz, 1H), 7.55 (dd, J=5.2, 1.2 Hz, 1H), 7.43-7.31 (m, 3H), 7.18 (dd, J=5.2, 4.0 Hz, 1H), 2.75 (s, 3H).

Compound 318: 2-(5-Phenyl-[1,3,4]oxadiazol-2-yl)-phenol

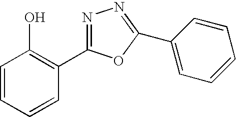

[M+H]⁺ 239.2; ¹H (400 MHz, DMSO) δ 10.27 (s, 1H), 8.09-8.06 (m, 2H), 7.90 (dd, J=4.0, 2.0 Hz, 1H), 7.63-7.58 (m, 3H), 7.46 (ddd, J=8.8, 7.6, 2.0 Hz, 1H), 7.09 (dd, J=8.0, 0.8 Hz, 1H), 7.03 (ddd, J=8.4, 7.6, 0.8 Hz, 1H).

Compound 319: 2-[5-(2-Fluoro-phenyl)-[1,3,4]oxadiazol-2-yl]-phenol

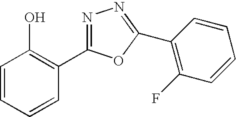

[M+H]⁺ 257.2; ¹H (400 MHz, DMSO) δ 10.28 (s, 1H), 8.09 (ddd, J=7.6, 7.2, 1.6 Hz, 1H), 7.85 (dd, J=7.6, 1.6 Hz, 1H), 7.71-7.65 (m, 1H), 7.50-7.41 (m, 3H), 7.09 (d, J=8.4 Hz, 1H), 7.02 (ddd, J=8.0, 8.0, 0.8 Hz, 1H).

Compound 321: 2-[5-(2-Nitro-phenyl)-[1,3,4]oxadiazol-2-yl]-phenol

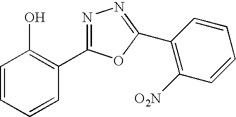

[M+H]⁺ 284.1; ¹H (400 MHz, DMSO) δ 10.35 (s, 1H), 8.17 (dd, J=8.0, 1.6 Hz, 1H), 8.12 (dd, J=7.6, 2.0 Hz, 1H), 7.96-7.88 (m, 2H), 7.80 (dd, J=8.0, 2.4 Hz, 1H), 7.46 (ddd, J=8.8, 7.6, 2.0 Hz, 1H), 7.08 (d, J=3.6 Hz, 1H), 7.00 (ddd, J=7.6, 7.6, 0.8 Hz, 1H).

Compound 320: 2-[5-(2-Trifluoromethyl-phenyl)-[1,3,4]oxadiazol-2-yl]-phenol

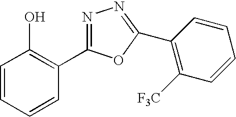

[M+H]⁺ 307.2; ¹H (400 MHz, DMSO) δ 10.31 (s, 1H), 8.13 (d, J=6.8 Hz, 1H), 8.00 (dd, J=8.0, 0.8 Hz, 1H), 7.93-7.86 (m, 2H), 7.80 (dd, J=7.6, 1.6 Hz, 1H), 7.46 (ddd, J=8.8, 7.2, 1.6 Hz, 1H), 7.09 (dd, J=8.4, 0.8 Hz, 1H), 7.01 (ddd, J=8.4, 7.6, 1.2 Hz, 1H).

Compound 322: 2-[5-(2,6-Dichloro-phenyl)-[1,3,4]oxadiazol-2-yl]-phenol

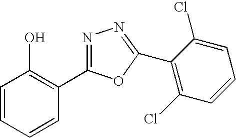

[M+H]⁺ 307.1; ¹H (400 MHz, DMSO) δ 10.40 (s, 1H), 7.85 (dd, J=8.4, 2.0 Hz, 1H), 7.76-7.73 (m, 3H), 7.47 (ddd, J=8.8, 7.2, 1.6 Hz, 1H), 7.09 (dd, J=8.8, 0.8 Hz, 1H), 7.00 (ddd, J=7.6, 7.6, 1.2 Hz, 1H).

Compound 342: 2-[5-(3-Trifluoromethyl-pyridin-2-yl)-[1,3,4]oxadiazol-2-yl]-phenol

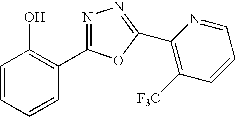

[M+H]⁺ 308.2; ¹H (400 MHz, DMSO) δ 10.38 (s, 1H), 9.08 (dd, J=4.8, 0.8 Hz, 1H), 8.51 (dd, J=8.4, 1.2 Hz, 1H), 7.94-7.91 (m, 1H), 7.84 (dd, J=8.0, 1.6 Hz, 1H), 7.48 (ddd, J=8.0, 6.8, 1.2 Hz, 1H), 7.10 (dd, J=8.4, 1.2 Hz, 1H), 7.02 (ddd, J=8.0, 8.0, 1.2 Hz, 1H).

Compound 343: 2-[5-(3-Chloro-5-trifluoromethyl-pyridin-2-yl)-[1,3,4]oxadiazol-2-yl]-phenol

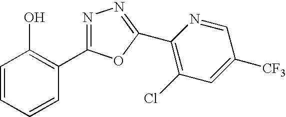

[M+H]⁺ 342.1; ¹H (400 MHz, DMSO) δ 10.40 (s, 1H), 9.18 (dd, J=2.0, 0.8 Hz, 1H), 8.77 (d, J=2.4 Hz, 1H), 7.86 (dd, J=7.6, 1.2 Hz, 1H), 7.48 (ddd, J=8.8, 7.2, 1.6 Hz, 1H), 7.10 (dd, J=8.4, 0.8 Hz, 1H), 7.03 (ddd, J=8.0, 8.0, 1.6 Hz, 1H).

Compound 323: 2-(5-Furan-2-yl-[1,3,4]oxadiazol-2-yl)-phenol

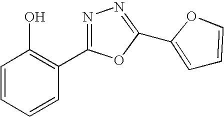

[M+H]⁺ 229.1; ¹H (400 MHz, DMSO) δ 10.27 (s, 1H), 8.06 (dd, J=1.6, 0.8 Hz, 1H), 7.82 (dd, J=7.6, 1.2 Hz, 1H), 7.46 (ddd, J=7.2, 4.8, 2.0 Hz, 1H), 7.40 (d, J=2.8 Hz, 1H), 7.08 (d, J=8.4 Hz, 1H), 7.01 (ddd, J=8.4, 7.6, 0.8 Hz, 1H), 6.80 (dd, J=3.2, 1.6 Hz, 1H).

Compound 324: 2-(5-Thiophen-2-yl-[1,3,4]oxadiazol-2-yl)-phenol

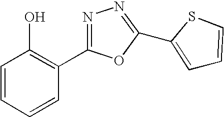

[M+H]⁺ 245.1; ¹H (400 MHz, DMSO) δ 10.26 (s, 1H), 7.94 (dd, J=5.2, 1.6 Hz, 1H), 7.88 (dd, J=4.0, 1.6 Hz, 1H), 7.83 (dd, J=8.0, 2.4 Hz, 1H), 7.45 (ddd, J=8.8, 7.2, 1.2 Hz, 1H), 7.30 (dd, J=4.8, 4.0 Hz, 1H), 7.08 (dd, J=8.4, 0.8 Hz, 1H), 7.01 (ddd, J=8.0, 8.0, 0.8 Hz, 1H).

Compound 325: 2-(2-Methoxy-phenyl)-5-phenyl-[1,3,4]oxadiazole

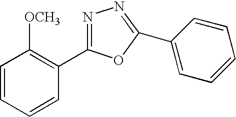

[M+H]⁺ 253.2; ¹H (400 MHz, DMSO) δ 8.06-8.04 (m, 2H), 7.93 (dd, J=7.6, 1.2 Hz, 1H), 7.62-7.57 (m, 4H), 7.26 (d, J=8.4 Hz, 1H), 7.13 (ddd, J=8.0, 8.0, 1.2 Hz, 1H), 3.93 (s, 3H).

Compound 326: 2-(2-Fluoro-phenyl)-5-(2-methoxy-phenyl)-[1,3,4]oxadiazole

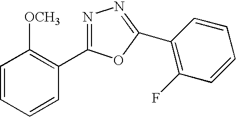

[M+H]⁺ 271.2; ¹H (400 MHz, DMSO) δ 8.07 (ddd, J=7.6, 7.2, 1.2 Hz, 1H), 7.91 (dd, J=7.6, 2.0 Hz, 1H), 7.70-7.65 (m, 1H), 7.60 (ddd, J=9.6, 7.6, 2.0 Hz, 1H), 7.50-7.41 (m, 2H), 7.26 (d, J=7.6 Hz, 1H), 7.13 (ddd, J=7.6, 7.6, 1.2 Hz, 1H), 3.91 (s, 3H).

Compound 328: 2-(2-Methoxy-phenyl)-5-(2-nitro-phenyl)-[1,3,4]oxadiazole

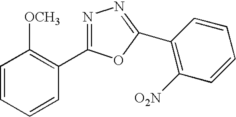

[M+H]⁺ 298.1; ¹H (400 MHz, DMSO) δ 8.17-8.11 (m, 2H), 7.96-7.87 (m, 3H), 7.62 (ddd, J=9.6, 7.2, 2.0 Hz, 1H), 7.26 (d, J=8.4 Hz, 1H), 7.13 (dd, J=8.8, 7.2 Hz, 1H), 3.90 (s, 3H).

Compound 327: 2-(2-Methoxy-phenyl)-5-(2-trifluoromethyl-phenyl)-[1,3,4]oxadiazole

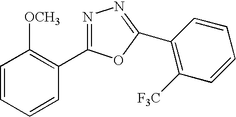

[M+H]⁺ 321.2; ¹H (400 MHz, DMSO) δ 8.12 (d, J=8.0 Hz, 1H), 8.00 (d, J=7.6 Hz, 1H), 7.93-7.86 (m, 3H), 7.61 (ddd, J=8.4, 7.2, 2.0 Hz, 1H), 7.27 (d, J=8.4 Hz, 1H), 7.14 (ddd, J=8.4, 7.6, 1.2 Hz, 1H), 3.90 (s, 3H).

Compound 329: 2-(2,6-Dichloro-phenyl)-5-(2-methoxy-phenyl)-[1,3,4]oxadiazole

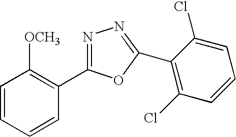

[M+H]⁺ 321.1; ¹H (400 MHz, DMSO) δ 7.90 (dd, J=8.0, 2.0 Hz, 1H), 7.77-7.72 (m, 3H), 7.63 (ddd, J=8.0, 7.2, 1.2 Hz, 1H), 7.28 (d, J=8.4 Hz, 1H), 7.14 (ddd, J=7.6, 7.6, 1.2 Hz, 1H), 3.89 (s, 3H).

Compound 344: 2-[5-(2-Methoxy-phenyl)-[1,3,4]oxadiazol-2-yl]-pyridine

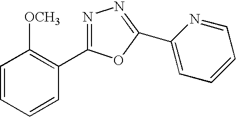

[M+H]⁺ 254.0; ¹H (400 MHz, DMSO) δ 8.79 (dd, J=7.6, 1.2 Hz, 1H), 8.21 (dd, J=8.0, 1.6 Hz, 1H), 8.05 (ddd, J=8.0, 7.6, 1.2 Hz, 1H), 7.91 (dd, J=8.0, 1.6 Hz, 1H), 7.95-7.60 (m, 2H), 7.28 (d, J=7.6 Hz, 1H), 7.15 (ddd, J=7.6, 7.2, 0.8 Hz, 1H), 3.92 (s, 3H).

Compound 345: 2-15-(2-Methoxy-phenyl)-[1,3,4]oxadiazol-2-yl]-3-trifluoromethyl-pyridine

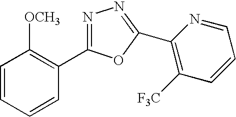

[M+H]⁺ 322.1; ¹H (400 MHz, DMSO) δ 9.07 (ddd, J=2.0, 1.6, 0.8 Hz, 1H), 8.51 (dd, J=8.8, 1.6 Hz, 1H), 7.94-7.90 (m, 2H), 7.64 (ddd, J=8.8, 7.6, 1.2 Hz, 1H), 7.29 (d, J=8.0 Hz, 1H), 7.15 (ddd, J=8.0, 7.6, 1.2 Hz, 1H), 3.90 (s, 3H).

Compound 346: 3-Chloro-2-[5-(2-methoxy-phenyl)-[1,3,4]oxadiazol-2-yl]-5-trifluoromethyl-pyridine

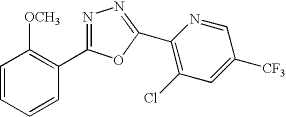

[M+H]⁺ 356.1; ¹H (400 MHz, DMSO) δ 9.16 (dd, J=2.0, 0.8 Hz, 1H), 8.75 (d, J=2.0 Hz, 1H), 7.91 (dd, J=7.6, 2.0 Hz, 1H), 7.63 (ddd, J=8.8, 7.6, 2.0 Hz, 1H), 7.28 (d, J=8.0 Hz, 1H), 7.15 (ddd, J=7.6, 7.6, 0.8 Hz, 1H), 3.91 (s, 3H).

Compound 330: 2-Furan-2-yl-5-(2-methoxy-phenyl)-[1,3,4]oxadiazole

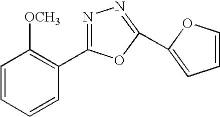

[M+H]⁺ 243.3; ¹H (400 MHz, DMSO) δ 8.05 (dd, J=2.0, 0.8 Hz, 1H), 7.87 (dd,J=7.6, 1.2 Hz, 1H), 7.60 (ddd, J=9.2, 7.6, 2.0 Hz, 1H), 7.37 (d, J=3.2 Hz, 1H), 7.26 (d, J=8.4 Hz, 1H), 7.12 (ddd, J=7.6, 7.2, 0.8 Hz, 1H), 6.79 (dd, J=3.6, 2.0 Hz, 1H), 3.91 (s, 3H).

Compound 331: 2-(2-Methoxy-phenyl)-5-thiophen-2-yl-[1,3,4]oxadiazole

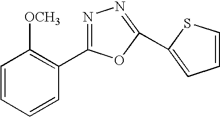

[M+H]⁺ 259.1; ¹H (400 MHz, DMSO) δ 7.93 (dd, J=4.8, 1.2 Hz, 1H), 7.89 (dd, J=8.0, 2.0 Hz, 1H), 7.85 (dd, J=4.0, 1.2 Hz, 1H), 7.59 (ddd, J=9.2, 7.2, 1.6 Hz, 1H), 7.29 (dd, J=5.2, 3.6 Hz, 1H), 7.25 (d, J=8.8 Hz, 1H), 7.12 (ddd, J=7.6, 7.6, 1.2 Hz, 1H), 3.91 (s, 3H).

Compound 332: 2-(2-Fluoro-phenyl)-5-phenyl-[1,3,4]oxadiazole

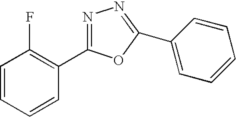

[M+H]⁺ 241.2; ¹H (400 MHz, DMSO) δ 8.11 (ddd, J=7.6, 7.2, 1.6 Hz, 1H), 8.06-8.04 (m, 2H), 7.70-7.64 (m, 1H), 7.62-7.57 (m, 3H), 7.49-7.40 (m, 2H).

Compound 333: 2,5-Bis-(2-fluoro-phenyl)-[1,3,4]oxadiazole

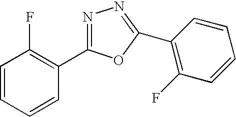

[M+H]⁺ 259.2; ¹H (400 MHz, DMSO) δ 8.08 (ddd, J=8.0, 7.2, 2.0 Hz, 2H), 7.71-7.65 (m, 2H), 7.49-7.40 (m, 4H).

Compound 334: 2-(2-Fluoro-phenyl)-5-(2-trifluoromethyl-phenyl)-[1,3,4]oxadiazole

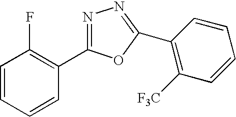

[M+H]⁺ 309.2; ¹H (400 MHz, DMSO) δ 8.14 (dd,J=8.0, 1.2 Hz, 1H), 8.05 (ddd,J=7.6, 7.2, 1.6 Hz, 1H), 8.00 (dd,J=7.6, 1.2 Hz, 1H), 7.93-7.87 (m, 2H), 7.71-7.67 (m, 1H), 7.49 (dd, J=8.4, 0.8 Hz, 1H), 7.45 (ddd,J=15.2, 7.6, 0.8 Hz, 1H).

Compound 335: 2-(2,6-Dichloro-phenyl)-5-(2-fluoro-phenyl)-[1,3,4]oxadiazole

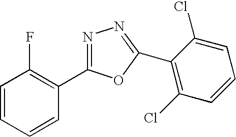

[M+H]⁺ 309.2; ¹H (400 MHz, DMSO) δ 8.10 (ddd, J=8.0, 7.2, 1.6 Hz, 1H), 7.78-7.70 (m, 4H), 7.51 (ddd, J=11.2, 8.4, 1.2 Hz, 1H), 7.45 (ddd, J=8.0, 7.2, 0.8 Hz, 1H).

Compound 347: 2-[5-(2-Fluoro-phenyl)-[1,3,4]oxadiazol-2-yl]-pyridine

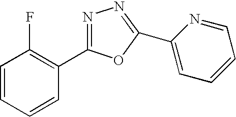

[M+H]⁺ 242.1; ¹H (400 MHz, DMSO) δ 8.80 (ddd, J=4.8, 2.0, 1.2 Hz, 1H), 8.24 (ddd, J=8.0, 1.2, 0.8 Hz, 1H), 8.12 (ddd, J=8.0, 7.6, 1.6 Hz, 1H), 8.07 (ddd, J=8.0, 8.0, 2.0 Hz, 1H), 7.74-7.68 (m, 1H), 7.65 (ddd, J=8.0, 5.2, 1.2 Hz, 1H), 7.50 (ddd, J=11.2, 8.0, 0.8 Hz, 1H), 7.45 (ddd, J=8.0, 7.2, 0.8 Hz, 1H).

Compound 348: 2-[5-(2-Fluoro-phenyl)-[1,3,4]oxadiazol-2-yl]-3-trifluoromethyl-pyridine

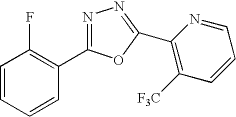

[M+H]⁺ 310.2; ¹H (400 MHz, DMSO) δ 9.09 (ddd, J=5.6, 1.6,0.8 Hz, 1H), 8.52 (dd, J=8.0, 0.8 Hz, 1H), 8.10 (ddd, J=7.2, 7.2, 1.6 Hz, 1H), 7.94 (ddd, J=8.0, 4.8, 0.8 Hz, 1H), 7.77-7.71 (m, 1H), 7.52 (ddd, J=10.8, 8.0, 0.8 Hz, 1H), 7.47 (ddd, J=7.6, 7.2, 0.8 Hz, 1H).

Compound 349: 3-Chloro-2-[5-(2-fluoro-phenyl)-[1,3,4]oxadiazol-2-yl]-5-trifluoromethyl-pyridine

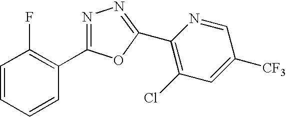

[M+H]⁺ 344.0; ¹H (400 MHz, DMSO) δ 9.18 (dd, J=2.0, 0.8 Hz, 1H), 8.77 (d, J=1.6 Hz, 1H), 8.10 (ddd, J=7.6, 7.2, 1.6 Hz, 1H), 7.76-7.70 (m, 1H), 7.53-7.44 (m, 2H).

Compound 336: 2-(2-Fluoro-phenyl)-5-furan-2-yl-[1,3,4]oxadiazole

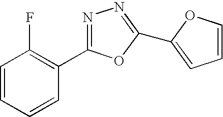

¹H (400 MHz, DMSO) δ 8.08-8.04 (m, 2H), 7.71-7.65 (m, 1H), 7.50-7.40 (m, 3H), 6.81 (dd, J=3.2, 2.0 Hz, 1H).

Compound 337: 2-(2-Fluoro-phenyl)-5-thiophen-2-yl-[1,3,4]oxadiazole

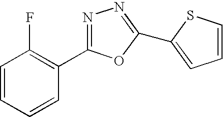

¹H (400 MHz, DMSO) δ 8.07 (ddd, J=8.0, 7.2, 1.6 Hz, 1H), 7.95 (dd, J=5.2, 1.2 Hz, 1H), 7.88 (dd, J=3.6, 0.8 Hz, 1H), 7.70-7.64 (m, 1H), 7.48-7.40 (m, 2H), 7.29 (dd, J=4.8, 3.6 Hz, 1H).

Triazoles

Scheme 10 shows a general procedure to prepare the 3,5-disubstituted triazoles.

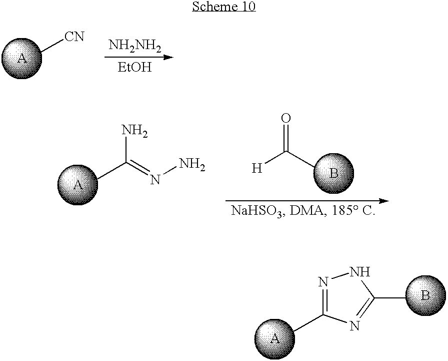

In an exemplary synthetic route, the aryl nitrile was first converted to N-aminoamidine when reacted with hydrazine hydrate in ethanol. The condensation of N-aminoamidine with aryl aldehyde in the presence of sodium hydrogen sulfite in N,N-dimethylacetamide at 185° C. provided 3,5-disubsituted triazoles.

Compound 389: 2-[5-(4-Isopropyl-phenyl)-1H-[1,2,4]triazol-3-yl]-pyridine

### EXAMPLE 35

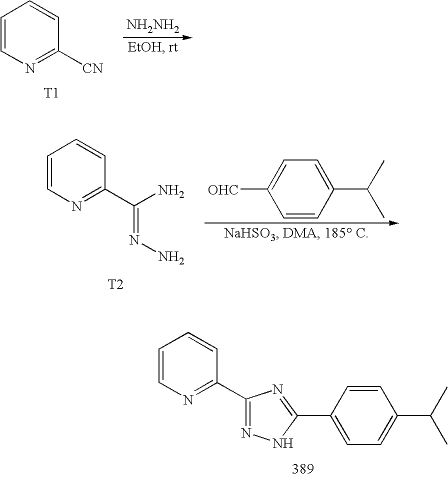

Step 1: Preparation of N-aino-pyridine-2-carboxamidine

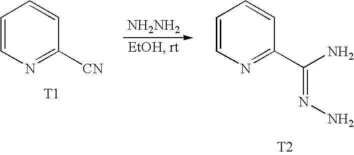

A mixture of pyridine-2-carbonitrile T1 (1.0 g, 10.0 mmol) and hydrazine (3.1 g, 100.0 mmol) in ethanol (5 mL) was stirred at room temperature for 2 hr. After removal of the solvent, the solid residue was partitioned between water and ethyl acetate. The aqueous layer was separated and extracted with ethyl acetate. The combined organic layers were washed with brine, dried over anhydrous magnesium sulfate and evaporated to dryness to yield a yellow solid T2 (1.2 g, 88%) that was used without further purification. [M+H]⁺ 137.2; 1H NMR (400 MHz, DMSO-D6) δ ppm 5.29 (bs, 2 H) 5.70 (bs, 2 H) 7.23-7.34 (m, 1 H) 7.64-7.76 (m, 1 H) 7.84-7.94 (m, 1 H) 8.40-8.51 (m, 1 H).

Step 2

Compound 389: 2-[5-(4-ispropyl-phenyl)-1-[1,2,4]triazol-3-yl]-pyridine

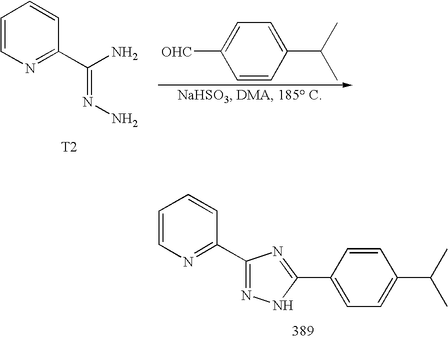

A mixture of N-amino-pyridine-2-carboxamidine T2 (68.1 mg, 0.5 mmol), 4-isopropyl-benzaldehyde (74.1 mg, 0.5 mmol) and sodium hydrogensulfite (78.0 mg, 0.75 mmol) in N,N-dimethylacetamide (1 mL) was heated at 185° C. for 2 hr. The reaction mixture was re-partitioned between ethyl acetate and water. The organic phase was washed with brine and dried over anhydrous magnesium sulfate. After removal of the solvent, the residue was purified using column chromatography (30% EtOAc-Hexane) to give a colorless solid 389 (115 mg, 87%). [M+H]⁺ 265.3; 1H NMR (400 MHz, CHLOROFORM-D) δ ppm 1.31 (d, J=6.60 Hz, 6 H) 2.87-3.05 (m, 1 H) 7.33 (d, J=8.07 Hz, 2 H) 7.37-7.46 (m, 1 H) 7.85-7.95 (m, 1 H) 8.15 (d, J=8.07 Hz, 2 H) 8.35 (d, J=7.70 Hz, 1 H) 8.83 (d, J=4.40 Hz, 1 H); ¹³C NMR ( MHz, CHLOROFORM-D) δ ppm 162.75, 155.26, 150.41, 149.56, 146.81, 137.88, 128.44, 126.89, 126.86, 125.00, 122.38, 34.48, and 24.35.

Compound 350: 2-(5-Naphthalen-1-yl-1H-[1,2,4]triazol-3-yl)-pyridine (Example 36)

A mixture of N-amino-pyridine-2-carboxamidine T2 (27.2 mg, 0.2 mmol), naphthalene-1-carbaldehyde (31.2 mg, 0.2 mmol) and sodium hydrogensulfite (31.2 mg, 0.3 mmol) in N,N-dimethylacetamide (0.4 mL) was heated at 185° C. for 2 hr. The reaction mixture was purified using preparative LC/MS to provide 2-(5-naphthalen-1-yl-1H-[1,2,4]triazol-3-yl)-pyridine. [M+H]⁺ 273.2; 1H NMR (400 MHz, DMSO-D6) δ ppm 7.54-7.70 (m, 4 H) 7.96-8.13 (m, 3 H) 8.17 (d, J=7.33 Hz, 1 H) 8.25 (d, J=7.70 Hz, 1 H) 8.76 (d, 1 H) 9.06 (d, J=8.43 Hz, 1 H).

Compound 351: 4-tert-Butyl-2-(5-pyridin-2-yl-2H-[1,2,4]triazol-3-yl)-phenol (Example 37)

4-tert-Butyl-2-(5-pyridin-2-yl-2H-[1,2,4]triazol-3-yl)-phenol was prepared from N-amino-pyridine-2-carboxamidine and 5-tert-Butyl-2-hydroxy-benzaldehyde using the protocol described in Example 36. [M+H]⁺ 295.3; 1H NMR (400 MHz, DMSO-D6) δ ppm 1.30 (s, 9 H) 6.94 (dd, J=8.43, 1.10 Hz, 1 H) 7.37 (dd, J=8.25, 2.02 Hz, 1 H) 7.54-7.62 (m, 1 H) 8.00-8.10 (m, 2 H) 8.22 (d, J=8.07 Hz, 1 H) 8.74 (d, J=4.77 Hz, 1 H).

Compound 352: 4-Nitro-2-(5-pyridin-2-yl-2H-[1,2,4]triazol-3-yl)-phenol (Example 38)

4-Nitro-2-(5-pyridin-2-yl-2H-[1,2,4]triazol-3-yl)-phenol was prepared from N-amino-pyridine-2-carboxamidine and 5-nitro-2-hydroxy-benzaldehyde using the protocol described in Example 1, step 2. [M+H]⁺ 284.1.

Compound 353: 4-Fluoro-2-(5-pyridin-2-yl-2H-[1,2,4]triazol-3-yl)-phenol (Example 39)

4-Fluoro-2-(5-pyridin-2-yl-2H-[1,2,4]triazol-3-yl)-phenol was prepared from N-amino-pyridine-2-carboxamidine and 5-fluoro-2-hydroxy-benzaldehyde using the protocol described in Example 36. [M+H]⁺ 257.1.

Compound 360: 4-Methoxy-2-(5-pyridin-2-yl-2H-[1,2,4]triazol-3-yl)-phenol (Example 40)

4-Methoxy-2-(5-pyridin-2-yl-2H-[1,2,4]triazol-3-yl)-phenol was prepared from N-amino-pyridine-2-carboxamidine and 5-methoxy-2-hydroxy-benzaldehyde using the protocol described in Example 36. [M+H]⁺ 269.3.

Compound 354: 4-Chloro-2-(5-pyridin-2-yl-2H-[1,2,4]triazol-3-yl)-phenol (Example 41)

4-Chloro-2-(5-pyridin-2-yl-2H-[1,2,4]triazol-3-yl)-phenol was prepared from N-amino-pyridine-2-carboxamidine and 5-chloro-2-hydroxy-benzaldehyde using the protocol described in Example 36. [M+H]⁺ 273.3.

Compound 355: 6-Ethoxy-2-(5-pyridin-2-yl-2H-[1,2,4]triazol-3-yl)-phenol (Example 42)

6-Ethoxy-2-(5-pyridin-2-yl-2H-[1,2,4]triazol-3-yl)-phenol was prepared from N-amino-pyridine-2-carboxamidine and 3-ethoxy-2-hydroxy-benzaldehyde using the protocol described in Example 36. [M+H]⁺ 283.2.

Compound 356: 5-Benzyloxy-2-(5-pyridin-2-yl-2H-[1,2,4]triazol-3-yl)-phenol (Example 43)

5-Benzyloxy-2-(5-pyridin-2-yl-2H-[1,2,4]triazol-3-yl)-phenol was prepared from N-amino-pyridine-2-carboxamidine and 4-benzyloxy-2-hydroxy-benzaldehyde using the protocol described in Example 36. [M+H]⁺ 345.2.

Compound 357: 2-[5-(2-Fluoro-biphenyl-4-yl)-1H-[1,2,4]triazol-3-yl]-pyridine (Example 44)

2-[5-(2-Fluoro-biphenyl-4-yl)-1H-[1,2,4]triazol-3-yl]-pyridine was prepared from N-amino-pyridine-2-carboxamidine and 2-fluoro-biphenyl-4-carbaldehyde using the protocol described in Example 36. [M+H]⁺ 317.2.

Compound 358: 4-Bromo-2-(5-pyridin-2-yl-2H-[1,2,4]triazol-3-yl)-phenol (Example 45)

4-Bromo-2-(5-pyridin-2-yl-2H-[1,2,4]triazol-3-yl)-phenol was prepared from N-amino-pyridine-2-carboxamidine and 5-bromo-2-hydroxy-benzaldehyde using the protocol described in Example 36. [M+H]⁺ 317.1.

Compound 359: 4-Methyl-2-(5-pyridin-2-yl-2H-[1,2,4]triazol-3-yl)-phenol (Example 46)

4-Methyl-2-(5-pyridin-2-yl-2H-[1,2,4]triazol-3-yl)-phenol was prepared from N-amino-pyridine-2-carboxamidine and 5-methyl-2-hydroxy-benzaldehyde using the protocol described in Example 36. [M+H]⁺ 253.2; 1H NMR (400 MHz, DMSO-D6) δ ppm 2.29 (s, 3 H) 6.91 (d, J=8.07 Hz, 1 H) 7.14 (dd, J=8.25, 2.02 Hz, 1 H) 7.56-7.63 (m, 1 H) 7.81-7.87 (m, 1 H) 8.04-8.12 (m, 1 H) 8.23 (dd, J=8.07, 0.73 Hz, 1 H).

Compound 363: 5-Methoxy-2-(5-pyridin-2-yl-2H-[1,2,4]triazol-3-yl)-phenol (Example 47)

5-Methoxy-2-(5-pyridin-2-yl-2H-[1,2,4]triazol-3-yl)-phenol was prepared from N-amino-pyridine-2-carboxamidine and 4-methoxy-2-hydroxy-benzaldehyde using the protocol described in Example 36. [M+H]⁺ 269.3.

Compound 361: 2-(5-pyridin-2-yl-2H-[1,2,4]triazol-3-yl)-phenol (Example 48)

2-(5-pyridin-2-yl-2H-[1,2,4]triazol-3-yl)-phenol was prepared from N-amino-pyridine-2-carboxamidine and 2-hydroxy-benzaldehyde using the protocol described in Example 36. [M+H]⁺ 239.2.

Compound 362: 6-Methoxy-2-(5-pyridin-2-yl-2H-[1,2,4]triazol-3-yl)-phenol (Example 49)

6-Methoxy-2-(5-pyridin-2-yl-2H-[1,2,4]triazol-3-yl)-phenol was prepared from N-amino-pyridine-2-carboxamidine and 3-methoxy-2-hydroxy-benzaldehyde using the protocol described in Example 36. [M+H]⁺ 269.3.

Compound 364: 2-[5-(3-Nitro-phenyl)-1H-[1,2,4]triazol-3-yl]-pyridine (Example 50)

2-[5-(3-Nitro-phenyl)-1H-[1,2,4]triazol-3-yl]-pyridine was prepared from N-amino-pyridine-2-carboxamidine and 3-nitro-benzaldehyde using the protocol described in Example 36. [M+H]⁺ 268.3.

Compound 365: 5-Diethylamino-2-(5-pyridin-2-yl-2H-[1,2,4]triazol-3-yl)-phenol (Example 51)

5-Diethylamino-2-(5-pyridin-2-yl-2H-[1,2,4]triazol-3-yl)-phenol was prepared from N-amino-pyridine-2-carboxamidine and 4-diethylamino-2-hydroxy-benzaldehyde using the protocol described in Example 36. [M+H]⁺ 310.2.

Compound 386: 4-Hydroxy-2-(5-pyridin-2-yl-2H-[1,2,4]triazol-3-yl)-phenol (Example 52)

4-Hydroxy-2-(5-pyridin-2-yl-2H-[1,2,4]triazol-3-yl)-phenol was prepared from N-amino-pyridine-2-carboxamidine and 2,5-dihydroxybenzaldehyde using the protocol described in Example 36. [M+H]⁺ 255.2.

Compound 387: 2-[5-(3-Methoxy-phenyl)-1H-[1,2,4]triazol-3-yl]-pyridine (Example 52)

2-[5-(3-Methoxy-phenyl)-1H-[1,2,4]triazol-3-yl]-pyridine was prepared from N-amino-pyridine-2-carboxamidine and 3-methoxy-benzaldehyde using the protocol described in Example 36. [M+H]⁺ 253.3.

Compound 388: 2-[5-(2-Fluoro-5-methoxy-phenyl)-1H-[1,2,4]triazol-3-yl]-pyridine (Example 54)

2-[5-(2-Fluoro-5-methoxy-phenyl)-1H-[1,2,4]triazol-3-yl]-pyridine was prepared from N-amino-pyridine-2-carboxamidine and 2-fluoro-5-methoxy-benzaldehyde using the protocol described in Example 36. [M+H]⁺ 271.3.

Compound 482: 1-(5-Pyridin-2-yl-2H-[1,2,4]triazol-3-yl)-naphthalen-2-ol (Example 55)

1-(5-Pyridin-2-yl-2H-[1,2,4]triazol-3-yl)-naphthalen-2-ol was prepared from N-amino-pyridine-2-carboxamidine and 2-hydroxy-naphthalene-1-carbaldehyde using the protocol described in Example 36. [M+H]⁺ 289.2; 1H NMR (400 MHz, DMSO-D6) δ ppm 7.27 (d, J=8.86 Hz, 1 H) 7.36 (t, J=6.87 Hz, 1 H) 7.48-7.62 (m, 2 H) 7.88 (dd, J=17.11, 8.25 Hz, 2 H) 8.03 (t, J=7.18 Hz, 1 H) 8.20 (d, J=7.64 Hz, 1 H) 8.75 (d, J=4.28 Hz, 1 H) 8.91 (s, 1 H).

Compound 619: 4,6-Dichloro -2-(5-pyridin-2-yl-2H-[1,2,4]triazol-3-yl)-phenol (Example 56)

4,6-Dichloro-2-(5-pyridin-2-yl-2H-[1,2,4]triazol-3-yl)-phenol was prepared from N-amino-pyridine-2-carboxamidine and 4,6-dichloro-2-hydroxy-benzaldehyde using the protocol described in Example 36. [M+H]⁺ 307.1. 1H NMR (400 MHz, DMSO-D6) δ ppm 7.51-7.63 (m, 1 H) 7.66 (s, 1 H) 7.96 (s, 1 H) 8.05 (t, J=7.22 Hz, 1 H) 8.26 (d, J=7.80 Hz, 1 H) 8.76 (d, J=4.29 Hz, 1 H) 11.75 (s, 1 H).

Compound 620: 4,6-Dibromo -2-(5-pyridin-2-yl-2H-[1,2,4]triazol-3-yl)-phenol (Example 57)

4,6-Dibromo-2-(5-pyridin-2-yl-2H-[1,2,4]triazol-3-yl)-phenol was prepared from N-amino-pyridine-2-carboxamidine and 4,6-dibromo-2-hydroxy-benzaldehyde using the protocol described in Example 36. [M+H]⁺ 397.0. 1H NMR (400 MHz, DMSO-D6) δ ppm 8.00-8.11 (m, 1 H) 8.32 (s, 1 H) 8.51 (t, J=6.83 Hz, 1 H) 8.58 (s, 1 H) 8.72 (d, J=8.19 Hz, 1 H) 9.21 (d, J=4.68 Hz, 1 H) 12.31 (s, 1 H).

Compound 621: 4-Chloro-6-bromo-2-(5-pyridin-2-yl-2H-[1,2,4]triazol-3-yl)-phenol (Example 58)

4-Chloro-6-bromo-2-(5-pyridin-2-yl-2H-[1,2,4]triazol-3-yl)-phenol was prepared from N-amino-pyridine-2-carboxamidine and 4-chloro6-bromo-2-hydroxy-benzaldehyde using the protocol described in Example 36. [M+H]⁺ 353.2. 1H NMR (400 MHz, DMSO-D6) δ ppm 8.01-8.10 (m, 1 H) 8.22 (s, 1 H) 8.43-8.55 (m, 2 H) 8.71 (d, J=7.02 Hz, 1 H) 9.21 (d, J=3.51 Hz, 1 H) 12.31 (s, 1 H).

Compound 431: 2-[5-(2-Bromo-phenyl)-1H-[1,2,4]triazol-3-yl]-pyridine (Example 59)

2-[5-(2-Bromo-phenyl)-1H-[1,2,4]triazol-3-yl]-pyridine was prepared from N-amino-pyridine-2-carboxamidine and 2-bromo-benzaldehyde using the protocol described in Example 36. [M+H]⁺ 302.2.

Compound 432: 2-[5-(3-Bromo-phenyl)-1H-[1,2,4]triazol-3-yl]-pyridine (Example 60)

2-[5-(3-Bromo-phenyl)-1H-[1,2,4]triazol-3-yl]-pyridine was prepared from N-amino-pyridine-2-carboxamidine and 3-bromo-benzaldehyde using the protocol described in Example 36. [M+H]⁺ 302.2.

Compound 433: 2-[5-(4-Bromo-phenyl)-1H-[1,2,4]triazol-3-yl]-pyridine (Example 61)

2-[5-(4-Bromo-phenyl)-1H-[1,2,4]triazol-3-yl]-pyridine was prepared from N-amino-pyridine-2-carboxamidine and 4-bromo-benzaldehyde using the protocol described in Example 36. [M+H]⁺ 302.2.

Compound 434: 2-[5-(2-Fluoro-phenyl)-1H-[1,2,4]triazol-3-yl]-pyridine (Example 62)

2-[5-(2-Fluoro-phenyl)-1H-[1,2,4]triazol-3-yl]-pyridine was prepared from N-amino-pyridine-2-carboxamidine and 2-fluoro-benzaldehyde using the protocol described in Example 36. [M+H]⁺ 241.2.

Compound 472: 2-[5-(2-Chloro-phenyl)-1H-[l,2,4]triazol-3-yl]-pyridine (Example 63)

2-[5-(2-Chloro-phenyl)-1H-[1,2,4]triazol-3-yl]-pyridine was prepared from N-amino-pyridine-2-carboxamidine and 2-chloro-benzaldehyde using the protocol described in Example 36. [M+H]⁺ 257.3.

Compound 435: 2-[5-(2,6-Dichloro -phenyl)-1H-[1,2,4]triazol-3-yl]-pyridine (Example 64)

2-[5-(2,6-Dichloro-phenyl)-1H-[1,2,4]triazol-3-yl]-pyridine was prepared from N-amino-pyridine-2-carboxamidine and 2,6-dichloro -benzaldehyde using the protocol described in Example 36. [M+H]⁺ 292.2.

Compound 436: 2-[5-(2-Fluoro-6-chloro-phenyl)-1H-[1,2,4]triazol-3-yl]-pyridine (Example 65)

2-[5-(2-Fluoro-6-chloro-phenyl)-1H-[1,2,4]triazol-3-yl]-pyridine was prepared from N-amino-pyridine-2-carboxamidine and 2-fluoro-6-chloro-benzaldehyde using the protocol described in Example 36. [M+H]⁺ 275.3.

Compound 384: 4-tert-Butyl-2-(5-isoquinolin-1-yl-2H-[1,2,4]triazol-3-yl)-phenol (Example 66)

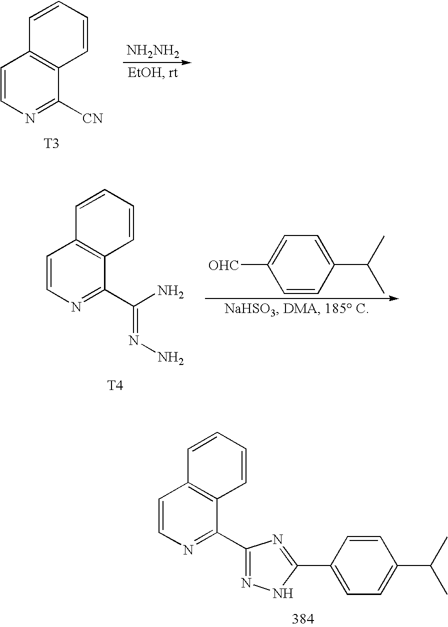

Step 1: PreparationofN-amino-isoquinoline-1-carboxamidine

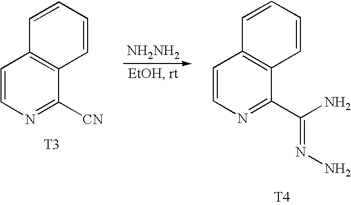

N-amino-isoquinoline-1-carboxamidine T4 was prepared from isoquinoline-1-carbonitrile T3 and hydrazine using the protocol described in Example 35, step 1. [M+H]⁺ 187.2; 1H NMR (400 MHz, DMSO-D6) δ ppm 5.52 (s, 2 H) 5.81 (s, 2 H) 7.59 (t, J=7.33 Hz, 1 H) 7.70 (t, J=7.15 Hz, 1 H) 7.74 (d, J=5.50 Hz, 1 H) 7.91 (d, J=8.07 Hz, 1 H) 8.42 (d, J=5.50 Hz, 1 H) 9.39 (d, J=8.80 Hz, 1 H).

Step 2: Preparation of 2-[5-(4-Isopropyl-phenyl)-1H-[ 1,2,4ltriazol-3-yl]-pyridine

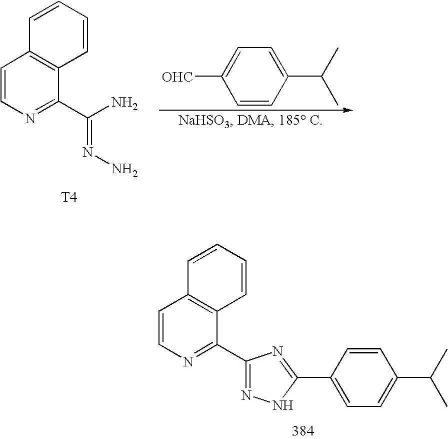

2-[5-(4-Isopropyl-phenyl)-1H-[1,2,4]triazol-3-yl]-pyridine was prepared from N-amino-isoquinoline-1-carboxamidine T4 and 4-isopropyl-benzaldehyde using the protocol described in Example 36. [M+H]⁺ 315.2.

Compound 366: 1-(5-Naphthalen-1-yl-1H-[1,2,4]triazol-3-yl)-isoquinoline (Example 67)

1-(5-Naphthalen-1-yl-1H-[1,2,4]triazol-3-yl)-isoquinoline was prepared from N-amino-isoquinoline-1-carboxamidine and naphthalene-1-carbaldehyde using the protocol described in Example 36. [M+H]⁺ 323.2.

Compound 367: 4-tert-Butyl-2-(5-isoquinolin-1-yl -2H-[1,2,4]triazol-3-yl)-phenol (Example 68)

4-tert-Butyl-2-(5-isoquinolin-1-yl-2H-[1,2,4]triazol-3-yl)-phenol was prepared from N-amino-isoquinoline-1-carboxamidine and 5-tert-Butyl-2-hydroxy-benzaldehyde using the protocol described in Example 36. [M+H]⁺ 345.3.

Compound 368: 4-Nitro-2-(5-isoquinolin-1-yl -2H-[1,2,4]triazol-3-yl)-phenol (Example 69)

4-Nitro-2-(5-isoquinolin-1-yl-2H-[1,2,4]triazol-3-yl)-phenol was prepared from N-amino-isoquinoline-1-carboxamidine and 5-nitro-2-hydroxy-benzaldehyde using the protocol described in Example 36. [M+H]⁺ 334.1.

Compound 369: 4-Fluoro-2-(5-isoquinolin-1-yl -2H-[1,2,4]triazol-3-yl)-phenol (Example 70)

4-Fluoro-2-(5-isoquinolin-1-yl-2H-[1,2,4]triazol-3-yl)-phenol was prepared from N-amino-isoquinoline-1-carboxamidine and 5-fluoro-2-hydroxy-benzaldehyde using the protocol described in Example 36. [M+H]⁺ 307.1.

Compound 370: 4-Methoxy-2-(5-isoquinolin-1-yl -2H-[1,2,4]triazol-3-yl)-phenol (Example 71)

4-Methoxy-2-(5-isoquinolin-1-yl-2H-[1,2,4]triazol-3-yl)-phenol was prepared from N-amino-isoquinoline-1-carboxamidine and 5-methoxy-2-hydroxy-benzaldehyde using the protocol described in Example 36. [M+H]⁺ 319.3.

Compound 371: 4-Chloro-2-(5-isoquinolin-1-yl -2H-[1,2,4]triazol-3-yl)-phenol (Example 72)

4-Chloro-2-(5-isoquinolin-1-yl-2H-[1,2,4]triazol-3-yl)-phenol was prepared from N-amino-isoquinoline-1-carboxamidine and 5-chloro-2-hydroxy-benzaldehyde using the protocol described in Example 36. [M+H]⁺ 323.3.

Compound 372: 6-Ethoxy-2-(5-isoquinolin-1-yl -2H-[1,2,4]triazol-3-yl)-phenol (Example 73)

6-Ethoxy-2-(5-isoquinolin-1-yl-2H-[1,2,4]triazol-3-yl)-phenol was prepared from N-amino-isoquinoline-1-carboxamidine and 3-ethoxy-2-hydroxy-benzaldehyde using the protocol described in Example 36. [M+H]⁺ 333.2.

Compound 373: 5-Benzyloxy-2-(5-isoquinolin-1-yl -2H-[1,2,4]triazol-3-yl)-phenol (Example 74)

5-Benzyloxy-2-(5-isoquinolin-1-yl-2H-[1,2,4]triazol-3-yl)-phenol was prepared from N-amino-isoquinoline-1-carboxamidine and 4-benzyloxy-2-hydroxy-benzaldehyde using the protocol described in Example 36. [M+H]⁺ 395.2.

Compound 374: 4-Bromo-2-(5-isoquinolin-1-yl -2H-[1,2,4]triazol-3-yl)-phenol (Example 75)

4-Bromo-2-(5-isoquinolin-1-yl-2H-[1,2,4]triazol-3-yl)-phenol was prepared from N-amino-isoquinoline-1-carboxamidine and 5-bromo-2-hydroxy-benzaldehyde using the protocol described in Example 36. [M+H]⁺ 367.1.

Compound 375: 4-Methyl-2-(5-isoquinolin-1-yl -2H-[1,2,4]triazol-3-yl)-phenol (Example 76)

4-Methyl-2-(5-isoquinolin-1-yl-2H-[1,2,4]triazol-3-yl)-phenol was prepared from N-amino-isoquinoline-1-carboxamidine and 5-methyl-2-hydroxy-benzaldehyde using the protocol described in Example 36. [M+H]⁺ 303.2.

Compound 376: 5-Methoxy-2-(5-isoquinolin-1-yl -2H-[1,2,4]triazol-3-yl)-phenol (Example 77)

5-Methoxy-2-(5-isoquinolin-1-yl-2H-[1,2,4]triazol-3-yl)-phenol was prepared from N-amino-isoquinoline-1-carboxamidine and 4-methoxy-2-hydroxy-benzaldehyde using the protocol described in Example 36. [M+H]⁺ 319.3.

Compound 377: 2-(5-isoquinolin-1-yl -2H-[1,2,4]triazol-3-yl)-phenol (Example 78)

2-(5-isoquinolin-1-yl-2H-[1,2,4]triazol-3-yl)-phenol was prepared from N-amino-isoquinoline-1-carboxamidine and 2-hydroxy-benzaldehyde using the protocol described in Example 36. [M+H]⁺ 289.2.

Compound 378: 6-Methoxy-2-(5-isoquinolin-1-yl -2H-[1,2,4]triazol-3-yl)-phenol (Example 79)

6-Methoxy-2-(5-isoquinolin-1-yl-2H-[1,2,4]triazol-3-yl)-phenol was prepared from N-amino-isoquinoline-1-carboxamidine and 3-methoxy-2-hydroxy-benzaldehyde using the protocol described in Example 36. [M+H]⁺ 319.3.

Compound 379: 2-[5-(3-Nitro-phenyl)-1H-[1,2,4]triazol-3-yl]-isoquinoline (Example 80)

2-[5-(3-Nitro-phenyl)-1H-[1,2,4]triazol-3-yl]-isoquinoline was prepared from N-amino-isoquinoline-1-carboxamidine and 3-nitro-benzaldehyde using the protocol described in Example 36. [M+H]⁺ 318.3.

Compound 380: 2-[5-(2,5-Dimethoxy-phenyl)-1H-[1,2,4]triazol-3-yl]-isoquinoline (Example 81)

2-[5-(2,5-Dimethoxy-phenyl)-1H-[1,2,4]triazol-3-yl]-isoquinoline was prepared from N-amino-isoquinoline-1-carboxamidine and 2,5-dimethoxy-benzaldehyde using the protocol described in Example 36. [M+H]⁺ 333.3.

Compound 381: 4-Hydroxy-2-(5-isoquinolin-1-yl -2H-[1,2,4]triazol-3-yl)-phenol (Example 82)

4-Hydroxy-2-(5-isoquinolin-1-yl-2H-[1,2,4]triazol-3-yl)-phenol was prepared from N-amino-isoquinoline-1-carboxamidine and 2,5-dihydroxybenzaldehyde using the protocol described in Example 36. [M+H]⁺ 305.2.

Compound 382: 2-[5-(3-Methoxy-phenyl)-1H-[1,2,4]triazol-3-yl]-isoquinoline (Example 83)

2-[5-(3-Methoxy-phenyl)-1H-[-1,2,4]triazol-3-yl]-isoquinoline was prepared from N-amino-isoquinoline-1-carboxamidine and 3-methoxy-benzaldehyde using the protocol described in Example 36. [M+H]⁺ 303.3.

Compound 383: 2-[5-(2-Fluoro-5-methoxy-phenyl)-1H-[1,2,4]triazol-3-yl]-isoquinoline (Example 84)

2-[5-(2-Fluoro-5-methoxy-phenyl)-1H-[1,2,4]triazol-3-yl]-isoquinoline was prepared from N-amino-isoquinoline-1-carboxamidine and 2-fluoro-5-methoxy-benzaldehyde using the protocol described in Example 36. [M+H]⁺ 321.3.

Compound 385: 1-(5-isoquinolin-1-yl -2H-[1,2,4]triazol-3-yl)-naphthalen-2-ol (Example 85)

1-(5-5-isoquinolin-1-yl-2H-[1,2,4]triazol-3-yl)-naphthalen-2-ol was prepared from N-amino-isoquinoline-1-carboxamidine and 2-hydroxy-naphthalene-1-carbaldehyde using the protocol described in Example 36. [M+H]⁺ 339.2.

Compound 437: 2-[5-(2-Bromo-phenyl)-1H-[1,2,4]triazol-3-yl]-isoquinoline (Example 86)

2-[5-(2-Bromo-phenyl)-1H-[1,2,4]triazol-3-yl]-isoquinoline was prepared from N-amino-isoquinoline-I-carboxamidine and 2-bromo-benzaldehyde using the protocol described in Example 36. [M+H]⁺ 352.2.

Compound 438: 2-[5-(3-Bromo-phenyl)-1H-[1,2,4]triazol-3-yl]-isoquinoline (Example 87)

2-[5-(3-Bromo-phenyl)-1H-[1,2,4]triazol-3-yl]-isoquinoline was prepared from N-amino-isoquinoline-1-carboxamidine and 3-bromo-benzaldehyde using the protocol described in Example 36. [M+H]⁺ 352.2.

Compound 439: 2-[5-(4-Bromo-phenyl)-1H-[1,2,4]triazol-3-yl]-isoquinoline (Example 88)

2-[5-(4-Bromo-phenyl)-1H-[1,2,4]triazol-3-yl]-isoquinoline was prepared from N-amino-isoquinoline-I-carboxamidine and 4-bromo-benzaldehyde using the protocol described in Example 36. [M+H]⁺ 352.2.

Compound 470: 2-[5-(2-Fluoro-phenyl)-1H-[1,2,4]triazol-3-yl]-isoquinoline (Example 89)

2-[5-(2-Fluoro-phenyl)-1H-[1,2,4]triazol-3-yl]-isoquinoline was prepared from N-amino-isoquinoline-1-carboxamidine and 2-fluoro-benzaldehyde using the protocol described in Example 36. [M+H]⁺ 291.2.

Compound 471: 2-[5-(2-Chloro-phenyl)-1H-[1,2,4]triazol-3-yl]-isoquinoline (Example 90)

2-[5-(2-Chloro-phenyl)-1H-[1,2,4]triazol-3-yl]-isoquinoline was prepared from N-amino-isoquinoline-1-carboxamidine and 2-chloro-benzaldehyde using the protocol described in Example 36. [M+H]⁺ 307.3.

Compound 408: 2-[5-(4-Isopropyl-phenyl)-1H-[1,2,4]triazol-3-yl]-6-methyl-pyridine (Example 91)

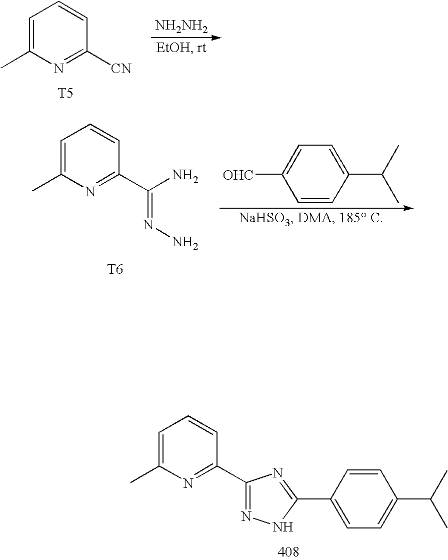

Step 1: Preparation of N-Amino-6-methyl-pyridine-2-carboxamidine T5

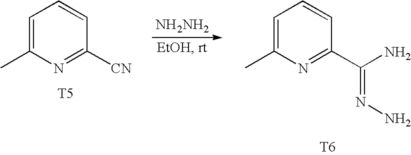

N-Amino-6-methyl-pyridine-2-carboxamidine T6 was prepared from 6-methyl-pyridine-2-carbonitrile T5 and hydrazine using the protocol described in Example 35, step 1. [M+H]⁺ 151.2; 1H NMR (400 MHz, DMSO-D6) δ ppm 2.46 (s, 3 H) 5.67 (s, 4 H) 7.13 (d, J=7.33 Hz, 1 H) 7.59 (t, J=7.70 Hz, 1 H) 7.68 (d, J=8.07 Hz, 1 H).

Step 2: Preparation of 2-[5-(4-Isopropyl-phenylD-1H-[I12,4]triazol-3-yl]-6-methyl pyridine

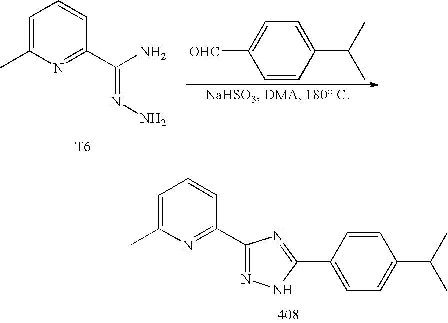

2-[5-(4-Isopropyl-phenyl)-1H-[1,2,4]triazol-3-yl]-6-methyl-pyridine was prepared from N-amino-6-methyl-pyridine-2-carboxamidine T6 and 4-isopropyl-benzaldehyde using the protocol described in Example 36. [M+H]⁺ 279.2.

Compound 390: 2-Methyl-6-(5-naphthalen-1-yl-1H-[1,2,4]triazol-3-yl)-pyridine (Example 92)

2-Methyl-6-(5-naphthalen-1-yl-1 H-[ 1,2,4]triazol-3-yl)-pyridine was prepared from N-amino-6-methyl-pyridine-2-carboxamidine and naphthalene-1-carbaldehyde using the protocol described in Example 36. [M+H]⁺ 287.2.

Compound 391: 4-tert-Butyl-2-[5-(6-methyl-pyridin-2-yl)-2H-[1,2,4]triazol-3-yl]-phenol (Example 93)

4-tert-Butyl-2-[5-(6-methyl-pyridin-2-yl)-2H-[1,2,4]triazol-3-yl]-phenol was prepared from N-amino-6-methyl-pyridine-2-carboxamidine and 5-tert-Butyl-2-hydroxy-benzaldehyde using the protocol described in Example 36. [M+H]⁺ 309.3.

Compound 392: 4-Nitro-2-(5-(6-methyl-pyridin-2-yl)-2H-[1,2,4]triazol-3-yl)-phenol (Example 94)

4-Nitro-2-(5-(6-methyl-pyridin-2-yl)-2H-[1,2,4]triazol-3-yl)-phenol was prepared from N-amino-6-methyl-pyridine-2-carboxamidine and 5-nitro-2-hydroxy-benzaldehyde using the protocol described in Example 36. [M+H]⁺ 298.1.

Compound 393: 4-Fluoro-2-(5-(6-methyl-pyridin-2-yl)-2H-[1,2,4]triazol-3-yl)-phenol (Example 94)

4-Fluoro-2-(5-(6-methyl-pyridin-2-yl)-2H-[1,2,4]triazol-3-yl)-phenol was prepared from N-amino-6-methyl-pyridine-2-carboxamidine and 5-fluoro-2-hydroxy-benzaldehyde using the protocol described Example 36. [M+H]⁺ 271.1.

Compound 394: 4-Methoxy-2-(5-(6-methyl-pyridin-2-yl)-2H-[1,2,4]triazol-3-yl)-phenol (Example 96)

4-Methoxy-2-(5-(6-methyl-pyridin-2-yl)-2H-[1,2,4]triazol-3-yl)-phenol was prepared from N-amino-6-methyl-pyridine-2-carboxamidine and 5-methoxy-2-hydroxy-benzaldehyde using the protocol described in Example 36. [M+H]⁺ 283.3.

Compound 396: 4-Chloro-2-(5-(6-methyl-pyridin-2-yl)-2H-[1,2,4]triazol-3-yl)-phenol (Example 97)

4-Chloro-2-(5-(6-methyl-pyridin-2-yl)-2H-[1,2,4]triazol-3-yl)-phenol was prepared from N-amino-6-methyl-pyridine-2-carboxamidine and 5-chloro-2-hydroxy-benzaldehyde using the protocol described in Example 36. [M+H]⁺ 287.3.

Compound 396: 6-Ethoxy-2-(5-(6-methyl-pyridin-2-yl)-2H-[1,2,4]triazol-3-yl)-phenol (Example 98)

6-Ethoxy-2-(5-(6-methyl-pyridin-2-yl)-2H-[1,2,4]triazol-3-yl)-phenol was prepared from N-amino-6-methyl-pyridine-2-carboxamidine and 3-ethoxy-2-hydroxy-benzaldehyde using the protocol described in Example 36. [M+H]⁺ 297.2.

Compound 397: 5-Benzyloxy-2-(5-(6-methyl-pyridin-2-yl)-2H-[1,2,4]triazol-3-yl)-phenol (Example 99)

5-Benzyloxy-2-(5-(6-methyl-pyridin-2-yl)-2H-[1,2,4]triazol-3-yl)-phenol was prepared from N-amino-6-methyl-pyridine-2-carboxamidine and 4-benzyloxy-2-hydroxy-benzaldehyde using the protocol described in Example 36. [M+H]⁺ 359.2.

Compound 398: 2-[5-(2-Fluoro-biphenyl-4-yi)-1H-[1,2,4]triazol-3-yl]-6-methyl-pyridine (Example 100)

2-[5-(2-Fluoro-biphenyl-4-yl)-1H-[1,2,4]triazol-3-yl]-6-methyl-pyridine was prepared from N-amino-6-methyl-pyridine-2-carboxamidine and 2-fluoro-biphenyl-4-carbaldehyde using the protocol described in Example 36. [M+H]⁺ 331.2.

Compound 399: 4-Bromo-2-(5-(6-methyl-pyridin-2-yl)-2H-[1,2,4]triazol-3-yl)-phenol (Example 101)

4-Bromo-2-(5-(6-methyl-pyridin-2-yl)-2H-[1,2,4]triazol-3-yl)-phenol was prepared from N-amino-6-methyl-pyridine-2-carboxamidine and 5-bromo-2-hydroxy-benzaldehyde using the protocol described in Example 36. [M+H]⁺ 331.1.

Compound 400: 4-Methyl-2-(5-(6-methyl-pyridin-2-yl)-2H-[1,2,4]triazol-3-yl)-phenol (Example 102)

4-Methyl-2-(5-(6-methyl-pyridin-2-yl)-2H-[1,2,4]triazol-3-yl)-phenol was prepared from N-amino-6-methyl-pyridine-2-carboxamidine and 5-methyl-2-hydroxy-benzaldehyde using the protocol described in Example 36. [M+H]⁺ 267.2.

Compound 401: 5-Methoxy-2-(5-(6-methyl-pyridin-2-yl)-2H-[1,2,4]triazol-3-yl)-phenol (Example 103)

5-Methoxy-2-(5-(6-methyl-pyridin-2-yl)-2H-[1,2,4]triazol-3-yl)-phenol was prepared from N-amino-6-methyl-pyridine-2-carboxamidine and 4-methoxy-2-hydroxy-benzaldehyde using the protocol described in Example 36. [M+H]⁺ 283.3.

Compound 402: 2-(5-(6-methyl-pyridin-2-yl)-2H-[1,2,4]triazol-3-yl)-phenol (Example 104)

2-(5-(6-methyl-pyridin-2-yl)-2H-[1,2,4]triazol-3-yl)-phenol was prepared from N-amino-6-methyl-pyridine-2-carboxamidine and 2-hydroxy-benzaldehyde using the protocol described in Example 36. [M+H]⁺ 253.2.

Compound 403: 6-Methoxy-2-(5-(6-methyl-pyridin-2-yl)-2H-[1,2,4]triazol-3-yl)-phenol (Example 105)

6-Methoxy-2-(5-(6-methyl-pyridin-2-yl)-2H-[1,2,4]triazol-3-yl)-phenol was prepared from N-amino-6-methyl-pyridine-2-carboxamidine and 3-methoxy-2-hydroxy-benzaldehyde using the protocol described in Example 36. [M+H]⁺ 283.3.

Compound 404: 6-Methyl -2-[5-(3-nitro-phenyl)-1H-[1,2,4]triazol-3-yl]-pyridine (Example 106)

6-Methyl-2-[5-(3-nitro-phenyl)-1H-[1,2,4]triazol-3-yl]-pyridine was prepared from N-amino-6-methyl-pyridine-2-carboxamidine and 3-nitro-benzaldehyde using the protocol described in Example 36. [M+H]⁺ 282.3.

Compound 405: 2-[5-(3-Methoxy-phenyl)-1H-[1,2,4]triazol-3-yl]-6-methyl-pyridine (Example 107)

2-[5-(3-Methoxy-phenyl)-1H-[1,2,4]triazol-3-yl]-6-methyl-pyridine was prepared from N-amino-6-methyl-pyridine-2-carboxamidine and 3-methoxy-benzaldehyde using the protocol described in Example 36. [M+H]⁺ 267.3.

Compound 406: 2-[5-(2-Fluoro-5-methoxy-phenyl)-1H-[1,2,4]triazol-3-yl]-6-methyl-pyridine (Example 108)

2-[5-(2-Fluoro-5-methoxy-phenyl)-1 H-[1,2,4]triazol-3-yl]-6-mehtyl-pyridine was prepared from N-amino-6-methyl-pyridine-2-carboxamidine and 2-fluoro-5-methoxy-benzaldehyde using the protocol described in Example 36. [M+H]⁺ 285.3.

Compound 407: 2-[5-(2-Methoxy-5-nitro-phenyl)-1H-[1,2,4]triazol-3-yl]-6-methyl-pyridine (Example 109)

2-[5-(2-Methoxy-5-nitro-phenyl)-1H-[1,2,4]triazol-3-yl]-6-methyl-pyridine was prepared from N-amino-6-methyl-pyridine-2-carboxamidine and 2-methoxy-5-nitro-benzaldehyde using the protocol described in Example 36. [M+H]⁺ 312.3.

Compound 409: 1-(5-(6-methyl-pyridin-2-yl)-2H-[1,2,4]triazol-3-yl)-naphthalen-2-ol (Example 101)

1-(5-(6-methyl-pyridin-2-yl)-2H-[1,2,4]triazol-3-yl)-naphthalen-2-ol was prepared from N-amino-6-methyl-pyridine-2-carboxamidine and 2-hydroxy-naphthalene-1-carbaldehyde using the protocol described in Example 36. [M+H]⁺ 303.2.

Compound 462: 2-[5-(2-Bromo-phenyl)-1H-[1,2,4]triazol-3-yl]-6-mehtyl-pyridine (Example 102)

2-[5-(2-Bromo-phenyl)-1H-[1,2,4]triazol-3-yl]-6-mehtyl-pyridine was prepared from N-amino-6-methyl-pyridine-2-carboxamidine and 2-bromo-benzaldehyde using the protocol described in Example 36. [M+H]⁺ 316.2.

Compound 463: 2-[5-(3-Bromo-phenyl)-1H-[1,2,4]triazol-3-yl]-6-methyl-pyridine (Example 112)

2-[5-(3-Bromo-phenyl)-1H-[1,2,4]triazol-3-yl]-6-methyl-pyridine was prepared from N-amino-6-methyl-pyridine-2-carboxamidine and 3-bromo-benzaldehyde using the protocol described in Example 36. [M+H]⁺ 316.2.

Compound 464: 2-[5-(4-Bromo-phenyl)-1H-[1,2,4]triazol-3-yl]-6-mehtyl-pyridine (Example 113)

2-[5-(4-Bromo-phenyl)-1H-[1,2,4]triazol-3-yl]-6-methyl-pyridine was prepared from N-amino-6-methyl-pyridine-2-carboxamidine and 4-bromo-benzaldehyde using the protocol described in Example 36. [M+H]⁺ 316.2.

Compound 465: 2-[5-(2-Fluoro-phenyl)-1H-[1,2,4]triazol-3-yl]-6-methyl-pyridine (Example 114)

2-[5-(2-Fluoro-phenyl)-1H-[1,2,4]triazol-3-yl]-6-methyl-pyridine was prepared from N-amino-6-methyl-pyridine-2-carboxamidine and 2-fluoro-benzaldehyde using the protocol described in Example 36. [M+H]⁺ 255.2.

Compound 466: 2-[5-(2-Chloro-phenyl)-1H-[1,2,4]triazol-3-yl]-6-methyl-pyridine (Example 115)

2-[5-(2-Chloro-phenyl)-1H-[1,2,4]triazol-3-yl]-6-methyl-pyridine was prepared from N-amino-6-methyl-pyridine-2-carboxamidine and 2-chloro-benzaldehyde using the protocol described in Example 36. [M+H]⁺ 271.3.

Compound 467: 2-[5-(2,6-Dichloro -phenyl)-1H-[1,2,4]triazol-3-yl]-6-methyl-pyridine (Example 116)

2-[5-(2,6-Dichloro-phenyl)-1H-[1,2,4]triazol-3-yl]-6-methyl-pyridine was prepared from N-amino-6-methyl-pyridine-2-carboxamidine and 2,6-dichloro -benzaldehyde using the protocol described in Example 36. [M+H]⁺ 306.2.

Compound 468: 2-[5-(2,6-Difluoro -phenyl)-1H-[1,2,4]triazol-3-yl]-6-methyl-pyridine (Example 117)

2-[5-(2,6-Difluoro-phenyl)-1H-[1,2,4]triazol-3-yl]-6-methyl-pyridine was prepared from N-amino-6-methyl-pyridine-2-carboxamidine and 2,6-difluoro -benzaldehyde using the protocol described in Example 36. [M+H]⁺ 273.2.

Compound 469: 2-[5-(2-Fluoro-6-chloro-phenyl)-1H-[1,2,4]triazol-3-yl]-6-methyl-pyridine (Example 118)

2-[5-(2-Fluoro-6-chloro-phenyl)-1H-[1,²,⁴]triazol-3-yl]-6-methyl-pyridine was prepared from N-amino-6-methyl-pyridine-2-carboxamidine and 2-fluoro-6-chloro-benzaldehyde using the protocol described in Example 36. [M+H]⁺ 289.3.

Compound 428: 3-[5-(4-Isopropyl-phenyl)-1H-[1,2,4]triazol-3-yl]-4-trifluoromethyl-pyridine (Example 119)

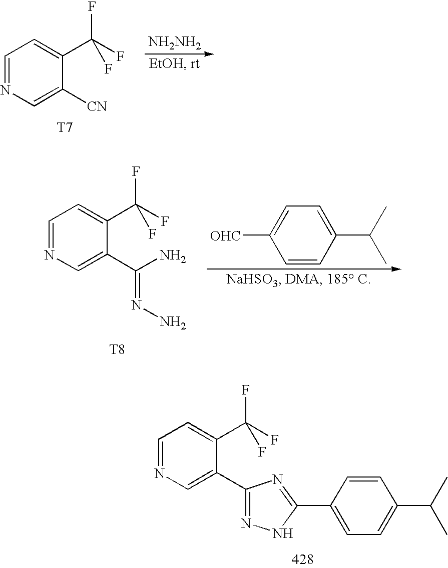

Step 1: Preparation of N-Amino-4-trifluoromethyl-nicotinamidine

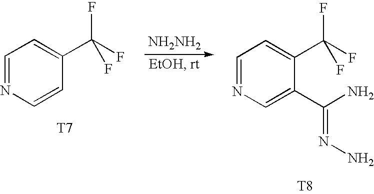

N-Amino-4-trifluoromethyl-nicotinamidine T8 was prepared from 4-trifluoromethyl-pyridine-3-carbonitrile T7 and hydrazine using the protocol described in Example 35, step 1. [M+H]⁺ 205.2; 1H NMR (400 MHz, DMSO-D6) δ ppm 5.01 (s, 2 H) 5.78 (s, 2 H) 7.69 (d, J=5.13 Hz, 1 H) 8.68 (s, 1 H) 8.75 (d, J=5.50 Hz, 1 H).

Step 2

Compound 85: 3-[5-(4-Isopropyl-phenyl)-1H-[1,2,4]triazol-3-yl]-4-trifluoromethyl-pyridine

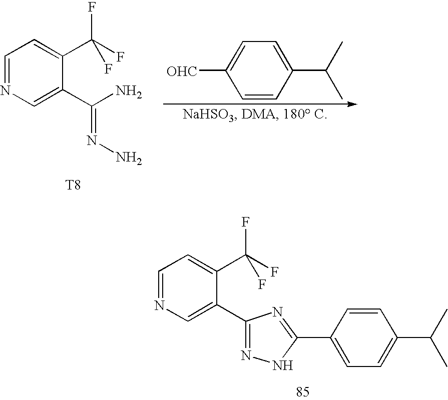

3-[5-(4-Isopropyl-phenyl)-1H-[1,2,4]triazol-3-yl]-4-trifluoromethyl-pyridine was prepared from N-amino-4-trifluoromethyl-nicotinamidine T8 and 4-isopropyl-benzaldehyde using the protocol described in Example 36. [M+H]⁺ 333.2.

Compound 410: 3-(5-Naphthalen-1-yl-1H-[1,2,4]triazol-3-yl)-4-trifluoromethyl-pyridine (Example 120)

3-(5-Naphthalen-1-yl-1H-[1,2,4]triazol-3-yl)-4-trifluoromethyl-pyridine was prepared from N-amino-4-trifluoromethyl-nicotinamidine and naphthalene-1-carbaldehyde using the protocol described in Example 36. [M+H]⁺ 341.2.

Compound 411: 4-tert-Butyl-2-[5-(4-trifluoromethyl-pyridin-3-yl)-2H-[1,2,4]triazol-3-yl]-phenol (Example 121)

4-tert-Butyl-2-[5-(4-trifluoromethyl-pyridin-3-yl)-2H-[1,2,4]triazol-3-yl]-phenol was prepared from N-amino-4-trifluoromethyl-nicotinamidine and 5-tert-Butyl-2-hydroxy-benzaldehyde using the protocol described in Example 36. [M+H]⁺ 363.3.

Compound 412: 4-Nitro-2-[5-(4-trifluoromethyl-pyridin-3-yl)-2H-[1,2,4]triazol-3-yl]-phenol (Example 122)

4-Nitro-2-[5-(4-trifluoromethyl-pyridin-3-yl)-2H-[1,2,4]triazol-3-yl]-phenol was prepared from N-amino-4-trifluoromethyl-nicotinamidine and 5-nitro-2-hydroxy-benzaldehyde using the protocol described in Example 36. [M+H]⁺ 352.1.

Compound 413: 4-Fluoro-2-[5-(4-trifluoromethyl-pyridin-3-yl)-2H-[1,2,4]triazol-3-yl]-phenol (Example 123)

4-Fluoro-2-[5-(4-trifluoromethyl-pyridin-3-yl)-2H-[1,2,4]triazol-3-yl]-phenol was prepared from N-amino-4-trifluoromethyl-nicotinamidine and 5-fluoro-2-hydroxy-benzaldehyde using the protocol described in Example 36. [M+H]⁺ 325.1.

Compound 414: 4-Methoxy-2-[5-(4-trifluoromethyl-pyridin-3-yl)-2H-[1,2,4]triazol-3-yl]-phenol (Example 124)

4-Methoxy-2-[5-(4-trifluoromethyl-pyridin-3-yl)-2H-[1,2,4]triazol-3-yl]-phenol was prepared from N-amino-4-trifluoromethyl-nicotinamidine and 5-methoxy-2-hydroxy-benzaldehyde using the protocol described in Example 36. [M+H]⁺ 336.3.

Compound 415: 4-Chloro-2-[5-(4-trifluoromethyl-pyridin-3-yl)-2H-[1,2,4]triazol-3-yl]-phenol (Example 125)

4-Chloro-2-[5-(4-trifluoromethyl-pyridin-3-yl)-2H-[1,2,4]triazol-3-yl]-phenol was prepared from N-amino-4-trifluoromethyl-nicotinamidine and 5-chloro-2-hydroxy-benzaldehyde using the protocol described in Example 36. [M+H]⁺ 341.3.

Compound 416: 6-Ethoxy-2-[5-(4-trifluoromethyl-pyridin-3-yl)-2H-[1,2,4]triazol-3-yl]-phenol (Example 126)

6-Ethoxy-2-[5-(4-trifluoromethyl-pyridin-3-yl)-2H-[1,2,4]triazol-3-yl]-phenol was prepared from N-amino-4-trifluoromethyl-nicotinamidine and 3-ethoxy-2-hydroxy-benzaldehyde using the protocol described in Example 36. [M+H]⁺ 351.2.

Compound 417: 5-Benzyloxy-2-[5-(4-trifluoromethyl-pyridin-3-yl)-2H-[1,2,4]triazol-3-yl]-phenol (Example 127)

5-Benzyloxy-2-[5-(4-trifluoromethyl-pyridin-3-yl)-2H-[1,2,4]triazol-3-yl]-phenol was prepared from N-amino-4-trifluoromethyl-nicotinamidine and 4-benzyloxy-2-hydroxy-benzaldehyde using the protocol described in Example 36. [M+H]⁺ 413.2.

Compound 418: 3-[5-(2-Fluoro-biphenyl-4-yl)-1H-[1,2,4]triazol-3-yl]-4-trifluoromethyl-pyridine (Example 128)

3-[5-(2-Fluoro-biphenyl-4-yl)-1H-[1,2,4]triazol-3-yl]-4-trifluoromethyl-pyridine was prepared from N-amino-4-trifluoromethyl-nicotinamidine and 2-fluoro-biphenyl-4-carbaldehyde using the protocol described in Example 36. [M+H]⁺ 385.2.

Compound 419: 4-Bromo-2-[5-(4-trifluoromethyl-pyridin-3-yl)-2H-[1,2,4]triazol-3-yl]-phenol (Example 129)

4-Bromo-2-[5-(4-trifluoromethyl-pyridin-3-yl)-2H-[1,2,4]triazol-3-yl]-phenol was prepared from N-amino-4-trifluoromethyl-nicotinamidine and 5-bromo-2-hydroxy-benzaldehyde using the protocol described in Example 36. [M+H]⁺ 386.1.

Compound 420: 4-Methyl-2-[5-(4-trifluoromethyl-pyridin-3-yl)-2H-[1,2,4]triazol-3-yl]-phenol (Example 130)

4-Methyl-2-[5-(4-trifluoromethyl-pyridin-3-yl)-2H-[1,2,4]triazol-3-yl]-phenol was prepared from N-amino-4-trifluoromethyl-nicotinamidine and 5-methyl-2-hydroxy-benzaldehyde using the protocol described in Example 36. [M+H]⁺ 321.2.

Compound 421: 5-Methoxy-2-[5-(4-trifluoromethyl-pyridin-3-yl)-2H-[1,2,4]triazol-3-yl]-phenol (Example 131)

5-Methoxy-2-[5-(4-trifluoromethyl-pyridin-3-yl)-2H-[1,2,4]triazol-3-yl]-phenol was prepared from N-amino-4-trifluoromethyl-nicotinamidine and 4-methoxy-2-hydroxy-benzaldehyde using the protocol described in Example 36. [M+H]⁺ 337.3.

Compound 422: 2-[5-(4-trifluoromethyl-pyridin-3-yl)-2H-[1,2,4]triazol-3-yl]-phenol (Example 132)

2-[5-(4-trifluoromethyl-pyridin-3-yl)-2H-[1,2,4]triazol-3-yl]-phenol was prepared from N-amino-4-trifluoromethyl-nicotinamidine and 2-hydroxy-benzaldehyde using the protocol described in Example 36. [M+H]⁺ 307.2.

Compound 423: 6-Methoxy-2-[5-(4-trifluoromethyl-pyridin-3-yl)-2H-[1,2,4]triazol-3-yl]-phenol (Example 133)

6-Methoxy-2-[5-(4-trifluoromethyl-pyridin-3-yl)-2H-[1,2,4]triazol-3-yl]-phenol was prepared from N-amino-4-trifluoromethyl-nicotinamidine and 3-methoxy-2-hydroxy-benzaldehyde using the protocol described in Example 36. [M+H]⁺ 337.3.

Compound 480: 3-[5-(3-Nitro-phenyl)-1H-[1,2,4]triazol-3-yl]-4-trifluoromethyl-pyridine (Example 134)

3-[5-(3-Nitro-phenyl)-1H-[1,2,4]triazol-3-yl]-4-trifluoromethyl-pyridine was prepared from N-amino-4-trifluoromethyl-nicotinamidine and 3-nitro-benzaldehyde using the protocol described in Example 36. [M+H]⁺ 336.3.

Compound 424: 5-Diethylamino-2-[5-(4-trifluoromethyl-pyridin-3-yl)-2H-[1,2,4]triazol-3-yl]-phenol (Example 135)

5-Diethylamino-2-[5-(4-trifluoromethyl-pyridin-3-yl)-2H-[1,2,4]triazol-3-yl]-phenol was prepared from N-amino-4-trifluoromethyl-nicotinamidine and 4-diethylamino-2-hydroxy-benzaldehyde using the protocol described in Example 36. [M+H]⁺ 378.2.

Compound 425: 1,1,7,7-Tetramethyl-9-[5-(4-trifluoromethyl-pyridin-3-yl)-2H-[1,2,4]triazol-3-yl]-2,3,6,7-tetrahydro-1H,5H-pyrido[3,2,1-ij]quinolin-8-ol (Example 136)

1,1,7,7-Tetramethyl-9-[5-(4-trifluoromethyl-pyridin-3-yl)-2H-[1,2,4]triazol-3-yl]-2,3,6,7-tetrahydro-1H,5H-pyrido[3,2,1-yl]quinolin-8-ol was prepared from N-amino-4-trifluoromethyl-nicotinamidine and 8-hydroxy-1,1,7,7-tetramethyl-2,3,6,7-tetrahydro-1H,5H-pyrido[3,2,1-yl]quinoline-9-carbaldehyde using the protocol described in Example 36. [M+H]⁺ 458.2.

Compound 426: 4-Hydroxy-2-[5-(4-trifluoromethyl-pyridin-3-yl)-2H-[1,2,4]triazol-3-yl]-phenol (Example 137)

4-Hydroxy-2-[5-(4-trifluoromethyl-pyridin-3-yl)-2H-[1,2,4]triazol-3-yl]-phenol was prepared from N-amino-4-trifluoromethyl-nicotinamidine and 2,5-dihydroxybenzaldehyde using the protocol described in Example 36. [M+H]⁺ 323.2.

Compound 427: 3-[5-(3-Methoxy-phenyl)-1H-[1,2,4]triazol-3-yl]-4-trifluoromethyl-pyridine (Example 138)

3-[5-(3-Methoxy-phenyl)-1H-[1,2,4]triazol-3-yl]-4-trifluoromethyl-pyridine was prepared from N-amino-4-trifluoromethyl-nicotinamidine and 3-methoxy-benzaldehyde using the protocol described in Example 36. [M+H]⁺ 321.3.

Compound 481: 3-[5-(2-Fluoro-5-methoxy-phenyl)-1H-[1,2,4]triazol-3-yl]-4-trifluoromethyl-pyridine (Example 139)

3-[5-(2-Fluoro-5-methoxy-phenyl)-1 H-[1,2,4]triazol-3-yl]-4-trifluoromethyl-pyridine was prepared from N-amino-4-trifluoromethyl-nicotinamidine and 2-fluoro-5-methoxy-benzaldehyde using the protocol described in Example 36. [M+H]⁺ 271.3.

Compound 429: 1-[5-(4-trifluoromethyl-pyridin-3-yl)-2H-[1,2,4]triazol-3-yl]-naphthalen-2-ol (Example 140)

1-[5-(4-trifluoromethyl-pyridin-3-yl)-2H-[1,2,4]triazol-3-yl]-naphthalen-2-ol was prepared from N-amino-4-trifluoromethyl-nicotinamidine and 2-hydroxy-naphthalene-1-carbaldehyde using the protocol described in Example 36. [M+H]⁺ 357.2.

Compound 473: 3-[5-(2-Bromo-phenyl)-1H-[1,2,4]triazol-3-yl]-4-trifluoromethyl-pyridine (Example 141)

3-[5-(2-Bromo-phenyl)-1H-[1,2,4]triazol-3-yl]-4-trifluoromethyl-pyridine was prepared from N-amino-4-trifluoromethyl-nicotinamidine and 2-bromo-benzaldehyde using the protocol described in Example 36. [M+H]⁺ 370.2.

Compound 474: 3-[5-(3-Bromo-phenyl)-1H-[1,2,4]triazol-3-yl]-4-trifluoromethyl-pyridine (Example 142)

3-[5-(3-Bromo-phenyl)-1H-[1,2,4]triazol-3-yl]-4-trifluoromethyl-pyridine was prepared from N-amino-4-trifluoromethyl-nicotinamidine and 3-bromo-benzaldehyde using the protocol described in Example 36. [M+H]⁺ 370.2.

Compound 430: 3-[5-(4-Bromo-phenyl)-1H-[1,2,4]triazol-3-yl]-4-trifluoromethyl-pyridine (Example 143)

3-[5-(4-Bromo-phenyl)-1H-[1,2,4]triazol-3-yl]-4-trifluoromethyl-pyridine was prepared from N-amino-4-trifluoromethyl-nicotinamidine and 4-bromo-benzaldehyde using the protocol described in Example 36. [M+H]⁺ 370.2.

Compound 475: 3-[5-(2-Fluoro-phenyl)-1H-[1,2,4]triazol-3-yl]-4-trifluoromethyl-pyridine (Example 144)

3-[5-(2-Fluoro-phenyl)-1H-[1,2,4]triazol-3-yl]-4-trifluoromethyl-pyridine was prepared from N-amino-4-trifluoromethyl-nicotinamidine and 2-fluoro-benzaldehyde using the protocol described in Example 36. [M+H]⁺ 309.2.

Compound 476: 3-[5-(2-Chloro-phenyl)-1H-[1,2,4]triazol-3-yl]-4-trifluoromethyl-pyridine (Example 145)

3-[5-(2-Chloro-phenyl)-1H-[1,2,4]triazol-3-yl]-4-trifluoromethyl-pyridine was prepared from N-amino-4-trifluoromethyl-nicotinamidine and 2-chloro-benzaldehyde using the protocol described in Example 36. [M+H]⁺ 325.3.

Compound 477: 3-[5-(2,6-Dichloro -phenyl)-1H-[1,2,4]triazol-3-yl]-4-trifluoromethyl-pyridine (Example 146)

3-[5-(2,6-Dichloro-phenyl)-1H-[1,2,4]triazol-3-yl]-4-trifluoromethyl-pyridine was prepared from N-amino-4-trifluoromethyl-nicotinamidine and 2,6-dichloro -benzaldehyde using the protocol described in Example 36. [M+H]⁺ 360.2.

Compound 478: 3-[5-(2,6-Difluoro -phenyl)-1H-[1,2,4]triazol-3-yl]-4-trifluoromethyl-pyridine (Example 147)

3-[5-(2,6-Difluoro-phenyl)-1H-[1,2,4]triazol-3-yl]-4-trifluoromethyl-pyridine was prepared from N-amino-4-trifluoromethyl-nicotinamidine and 2,6-difluoro -benzaldehyde using the protocol described in Example 36. [M+H]⁺ 327.2.

Compound 479: 3-[5-(2-Fluoro-6-chloro-phenyl)-1H-[1,2,4]triazol-3-yl]-4-trifluoromethyl-pyridine (Example 148)

3-[5-(2-Fluoro-6-chloro-phenyl)-1H-[1,2,4]triazol-3-yl]-4-trifluoromethyl-pyridine was prepared from N-amino-4-trifluoromethyl-nicotinamidine and 2-fluoro-6-chloro-benzaldehyde using the protocol described in Example 36. [M+H]⁺ 343.3.

Compound 460: 2-[5-(4-Isopropyl-phenyl)-1H-[1,2,4]triazol-3-yl]-3-methyl-pyridine 460 (Example 149)

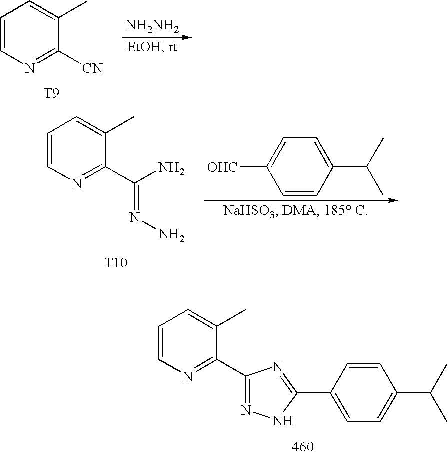

Step 1: Preparation of N-Amino-3-methyl-pyridine-2-carboxamidine

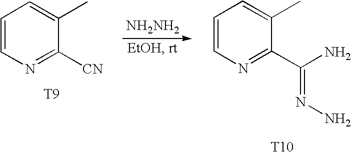

N-Amino-3-methyl-pyridine-2-carboxamidine 010 was prepared from 3-methyl-pyridine-2-carbonitrile O9 and hydrazine using the protocol described in Example 1, step 1. [M+H]⁺ 151.2; 1H NMR (400 MHz, DMSO-D6) δ ppm 2.49 (s, 3 H) 5.22 (s, 2 H) 5.62 (s, 2 H) 7.20 (dd, J=7.52, 4.58 Hz, 1 H) 7.57 (d, J=7.70 Hz, 1 H) 8.34 (dd, J=4.58, 0.92 Hz, 1 H).

Step 2: Preparation of 2-[5-(4-Isopropyl-phenyl)-1H-rl12,4ltriazol-3-yl]-3-methyl-pyridine

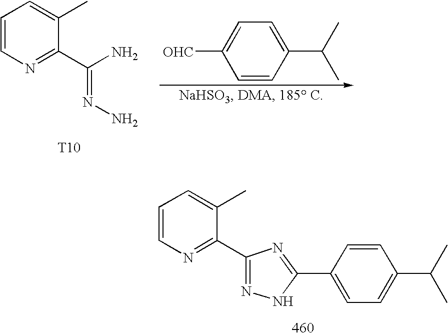

2-[5-(4-Isopropyl-phenyl)-1H-[1,2,4]triazol-3-yl]-3-methyl-pyridine was prepared from N-amino-6-methyl-pyridine-2-carboxamidine T10 and 4-isopropyl-benzaldehyde using the protocol described in Example 36. [M+H]⁺ 279.2.

Compound 440: 3-Methyl-2-(5-naphthalen-1-yl-1H-[1,2,4]triazol-3-yl)-pyridine (Example 150)

3-Methyl-2-(5-naphthalen-1-yl-1H-[1,2,4]triazol-3-yl)-pyridine was prepared from N-amino-3-methyl-pyridine-2-carboxamidine and naphthalene-1-carbaldehyde using the protocol described in Example 36. [M+H]⁺ 287.2.

Compound 441: 4-tert-Butyl-2-[5-(3-methyl-pyridin-2-yl)-2H-[1,2,4]triazol-3-yl]-phenol (Example 151 )

4-tert-Butyl-2-[5-(3-methyl-pyridin-2-yl)-2H-[1,2,4]triazol-3-yl]-phenol was prepared from N-amino-3-methyl-pyridine-2-carboxamidine and 5-tert-Butyl-2-hydroxy-benzaldehyde using the protocol described in Example 36. [M+H]⁺ 309.3.

Compound 442: 4-Nitro-2-(5-(3-methyl-pyridin-2-yl)-2H-[1,2,4]triazol-3-yl)-phenol (Example 152)

4-Nitro-2-(5-(3-methyl-pyridin-2-yl)-2H-[1,2,4]triazol-3-yl)-phenol was prepared from N-amino-3-methyl-pyridine-2-carboxamidine and 5-nitro-2-hydroxy-benzaldehyde using the protocol described in Example 36. [M+H]⁺ 298.1.

Compound 443: 4-Fluoro-2-(5-(3-methyl-pyridin-2-yl)-2H-[1,2,4]triazol-3-yl)-phenol (Example 153)

4-Fluoro-2-(5-(3-methyl-pyridin-2-yl)-2H-[1,2,4]triazol-3-yl)-phenol was prepared from N-amino-3-methyl-pyridine-2-carboxamidine and 5-fluoro-2-hydroxy-benzaldehyde using the protocol described in Example 36. [M+H]⁺ 271.1.

Compound 444: 4-Methoxy-2-(5-(3-methyl-pyridin-2-yl)-2H-[1,2,4]triazol-3-yl)-phenol (Example 154)

4-Methoxy-2-(5-(3-methyl-pyridin-2-yl)-2H-[1,2,4]triazol-3-yl)-phenol was prepared from N-amino-3-methyl-pyridine-2-carboxamidine and 5-methoxy-2-hydroxy-benzaldehyde using the protocol described in Example 36. [M+H]⁺ 283.3.

Compound 445: 4-Chloro-2-(5-(3-methyl-pyridin-2-yl)-2H-[1,2,4]triazol-3-yl)-phenol (Example 155)

4-Chloro-2-(5-(3-methyl-pyridin-2-yl)-2H-[1,2,4]triazol-3-yl)-phenol was prepared from N-amino-3-methyl-pyridine-2-carboxamidine and 5-chloro-2-hydroxy-benzaldehyde using the protocol described in Example 36. [M+H]⁺ 287.3.

Compound 446: 6-Ethoxy-2-(5-(3-methyl-pyridin-2-yl)-2H-[1,2,4]triazol-3-yl)-phenol (Example 156)

6-Ethoxy-2-(5-(3-methyl-pyridin-2-yl)-2H-[1,2,4]triazol-3-yl)-phenol was prepared from N-amino-3-methyl-pyridine-2-carboxamidine and 3-ethoxy-2-hydroxy-benzaldehyde using the protocol described in Example 36. [M+H]⁺ 297.2.

Compound 447: 5-Benzyloxy-2-(5-(3-methyl-pyridin-2-yl)-2H-[1,2,4]triazol-3-yl)-phenol (Example 157)

5-Benzyloxy-2-(5-(3-methyl-pyridin-2-yl)-2H-[1,2,4]triazol-3-yl)-phenol was prepared from N-amino-3-methyl-pyridine-2-carboxamidine and 4-benzyloxy-2-hydroxy-benzaldehyde using the protocol described in Example 36. [M+H]⁺ 359.2.

Compound 448: 2-[5-(2-Fluoro-biphenyl-4-yl)-1H-[1,2,4]triazol-3-yl]-3-methyl-pyridine (Example 158)

2-[5-(2-Fluoro-biphenyl-4-yl)-1H-[1,2,4]triazol-3-yl]-3-methyl-pyridine was prepared from N-amino-3-methyl-pyridine-2-carboxamidine and 2-fluoro-biphenyl-4-carbaldehyde using the protocol described in Example 36. [M+H]⁺ 331.2.

Compound 449: 4-Bromo-2-(5-(3-methyl-pyridin-2-yl)-2H-[1,2,4]triazol-3-yl)-phenol (Example 159)

4-Bromo-2-(5-(3-methyl-pyridin-2-yl)-2H-[1,2,4]triazol-3-yl)-phenol was prepared from N-amino-3-methyl-pyridine-2-carboxamidine and 5-bromo-2-hydroxy-benzaldehyde using the protocol described in Example 36. [M+H]⁺ 332.1.

Compound 450: 4-Methyl-2-(5-(3-methyl-pyridin-2-yl)-2H-[1,2,4]triazol-3-yl)-phenol (Example 160)

4-Methyl-2-(5-(3-methyl-pyridin-2-yl)-2H-[1,2,4]triazol-3-yl)-phenol was prepared from N-amino-3-methyl-pyridine-2-carboxamidine and 5-methyl-2-hydroxy-benzaldehyde using the protocol described in Example 36. [M+H]⁺ 267.2.

Compound 451: 5-Methoxy-2-(5-(3-methyl-pyridin-2-yl)-2H-[1,2,4]triazol-3-yl)-phenol (Example 161)

5-Methoxy-2-(5-(3-methyl-pyridin-2-yl)-2H-[1,2,4]triazol-3-yl)-phenol was prepared from N-amino-3-methyl-pyridine-2-carboxamidine and 4-methoxy-2-hydroxy-benzaldehyde using the protocol described in Example 36. [M+H]⁺ 283.3.

Compound 452: 2-(5-(3-methyl-pyridin-2-yl)-2H-[1,2,4]triazol-3-yl)-phenol (Example 162)

2-(5-(3-methyl-pyridin-2-yl)-2H-[1,2,4]triazol-3-yl)-phenol was prepared from N-amino-3-methyl-pyridine-2-carboxamidine and 2-hydroxy-benzaldehyde using the protocol described in Example 36. [M+H]⁺ 253.2.

Compound 453: 6-Methoxy-2-(5-(3-methyl-pyridin-2-yl)-2H-[1,2,4]triazol-3-yl)-phenol (Example 163)

6-Methoxy-2-(5-(3-methyl-pyridin-2-yl)-2H-[1,2,4]triazol-3-yl)-phenol was prepared from N-amino-3-methyl-pyridine-2-carboxamidine and 3-methoxy-2-hydroxy-benzaldehyde using the protocol described in Example 36. [M+H]⁺ 283.3.

Compound 454: 3-Methyl -2-[5-(3-nitro-phenyl)-1H-[1,2,4]triazol-3-yl]-pyridine (Example 164)

3-Methyl-2-[5-(3-nitro-phenyl)-1H-[1,2,4]triazol-3-yl]-pyridine was prepared from N-amino-3-methyl-pyridine-2-carboxamidine and 3-nitro-benzaldehyde using the protocol described in Example 36. [M+H]⁺ 282.3.

Compound 455: 5-Diethylamino-2-[5-(3-methyl-pyridin-2-yl)-2H-[1,2,4]triazol-3-yl]-phenol (Example 165)

5-Diethylamino-2-[5-(3-methyl-pyridin-2-yl)-2H-[1,2,4]triazol-3-yl]-phenol was prepared from N-amino-3-methyl-pyridine-2-carboxamidine and 4-diethylamino-2-hydroxy-benzaldehyde using the protocol described in Example 36. [M+H]⁺ 324.2.

Compound 456: 1,1,7,7-Tetramethyl-9-[5-(3-methyl-pyridin-2-yl)-2H-[1,2,4]triazol-3-yl]-2,3,6,7-tetrahydro-1H,5H-pyrido[3,2,1-ij]quinolin-8-ol (Example 166)

1,1,7,7-Tetramethyl-9-[5-(3-methyl-pyridin-2-yl)-2H-[1,2,4]triazol-3-yl]-2,3,6,7-tetrahydro-1H,5H-pyrido[3,2,1-ij]quinolin-8-ol was prepared from N-amino-3-methyl-pyridine-2-carboxamidine and 8-hydroxy-1,1,7,7-tetramethyl-2,3,6,7-tetrahydro-1H,5H-pyrido[3,2,1-ij]quinoline-9-carbaldehyde using the protocol described in Example 36. [M+H]⁺ 404.2.

Compound 457: 4-Hydroxy-2-[5-(3-methyl-pyridin-2-yl)-2H-[1,2,4]triazol-3-yl]-phenol (Example 167)

4-Hydroxy-2-[5-(3-methyl-pyridin-2-yl)-2H-[1,2,4]triazol-3-yl]-phenol was prepared from N-amino-3-methyl-pyridine-2-carboxamidine and 2,5-dihydroxybenzaldehyde using the protocol described in Example 36. [M+H]⁺ 269.2.

Compound 458: 2-[5-(3-Methoxy-phenyl)-1H-[1,2,4]triazol-3-yl]-3-methyl-pyridine (Example 168)

2-[5-(3-Methoxy-phenyl)-1H-[1,2,4]triazol-3-yl]-3-methyl-pyridine was prepared from N-amino-3-methyl-pyridine-2-carboxamidine and 3-methoxy-benzaldehyde using the protocol described in Example 36. [M+H]⁺ 267.3.

Compound 459: 2-[5-(2-Fluoro-5-methoxy-phenyl)-1H-[1,2,4]triazol-3-yl]-3-methyl-pyridine (Example 169)

2-[5-(2-Fluoro-5-methoxy-phenyl)-1H-[1,2,4]triazol-3-yl]-3-mehtyl-pyridine was prepared from N-amino-3-methyl-pyridine-2-carboxamidine and 2-fluoro-5-methoxy-benzaldehyde using the protocol described in Example 36. [M+H]⁺ 285.3.

Compound 461: 1-(5-(3-methyl-pyridin-2-yl)-2H-[1,2,4]triazol-3-yl)-naphthalen-2-ol (Example 170)

1-(5-(3-methyl-pyridin-2-yl)-2H-[1,2,4]triazol-3-yl)-naphthalen-2-ol was prepared from N-amino-3-methyl-pyridine-2-carboxamidine and 2-hydroxy-naphthalene-1-carbaldehyde using the protocol described in Example 36. [M+H]⁺ 303.2.

Compound 622: 4,6-Dichloro -2-[5-(3-methyl-pyridin-2-yl)-2H-[1,2,4]triazol-3-yl]-phenol (Example 171)

4,6-Dichloro-2-[5-(3-methyl-pyridin-2-yl)-2H-[1,2,4]triazol-3-yl]-phenol was prepared from N-amino-3-methyl-pyridine-2-carboxamidine and 4,6-dichloro-2-hydroxy-benzaldehyde using the protocol described in Example 36. [M+H]⁺ 322.2.

Compound 623: 4,6-Dibromo -2-[5-(3-methyl-pyridin-2-yl)-2H-[1,2,4]triazol-3-yl]-phenol (Example 172)

4,6-Dibromo-2-[5-(3-methyl-pyridin-2-yl)-2H-[1,2,4]triazol-3-yl]-phenol was prepared from N-amino-3-methyl-pyridine-2-carboxamidine and 4,6-dibromo-2-hydroxy-benzaldehyde using the protocol described in Example 36. [M+H]⁺ 412.2. 1H NMR (400 MHz, DMSO-D6) δ ppm 2.74 (s, 3 H) 7.85 (d, J=1.56 Hz, 1 H) 7.90 (d, J=7.80 Hz, 1 H) 8.10 (s, 1 H) 8.60 (d, J=4.29 Hz, 1 H) 12.09 (s, 1 H).

Compound 624: 4-Chloro-6-bromo-2-[5-(3-methyl-pyridin-2-yl)-2H-[1,2,4]triazol-3-yl]-phenol (Example 173)

4-Chloro-6-bromo-2-[5-(3-methyl-pyridin-2-yl)-2H-[1,2,4]triazol-3-yl]-phenol was prepared from N-amino-3-methyl-pyridine-2-carboxamidine and 4-chloro6-bromo-2-hydroxy-benzaldehyde using the protocol described in Example 36. [M+H]⁺ 368.2. 1H NMR (400 MHz, DMSO-D6) δ ppm 2.74 (s, 3 H) 7.52 (dd, J=7.22, 4.88 Hz, 1 H) 7.76 (d, J=1.56 Hz, 1 H) 7.90 (d, J=7.41 Hz, 1 H) 7.99 (s, 1 H) 8.61 (d, J=4.68 Hz, 1 H) 12.08 (s, 1 H).

Compound 504: 2-[5-(2-Bromo-phenyl)-1H-[1,2,4]triazol-3-yl]-3-mehtyl-pyridine (Example 174)

2-[5-(2-Bromo-phenyl)-1H-[1,2,4]triazol-3-yl]-3-mehtyl-pyridine was prepared from N-amino-3-methyl-pyridine-2-carboxamidine and 2-bromo-benzaldehyde using the protocol described in Example 36. [M+H]⁺ 316.2.

Compound 505: 2-[5-(3-Bromo-phenyl)-1H-[1,2,4]triazol-3-yl]-3-methyl-pyridine (Example 175)

2-[5-(3-Bromo-phenyl)-1H-[1,2,4]triazol-3-yl]-3-methyl-pyridine was prepared from N-amino-3-methyl-pyridine-2-carboxamidine and 3-bromo-benzaldehyde using the protocol described in Example 36. [M+H]⁺ 316.2.

Compound 502: 3-Bromo:2-[5-(4-isopropyl-phenyl)-1H-[1,2,4]triazol-3-yl]-pyridine (Example 176)

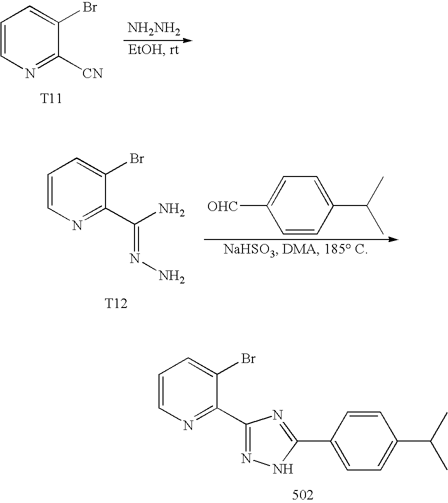

Step 1: Preparation of N-Amino-3-bromo-pyridine-2-carboxamidine

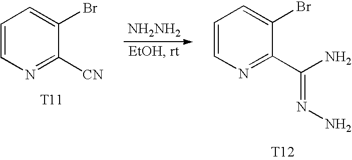

N-Amino-3-methyl-pyridine-2-carboxamidine T12 was prepared from 3-bromo-pyridine-2-carbonitrile T11 and hydrazine using the protocol described in Example 1, step 1. [M+H]⁺ 216.2.

Step 2: Preparation of 3-bromo2-[5-(4-isopropyl-phenyl)-1H-[l12,41triazol-3-yl]-pyridine

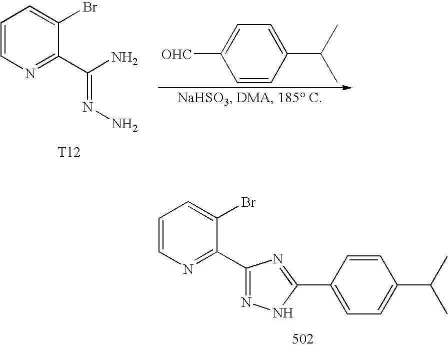

3-Bromo-2-[5-(4-isopropyl-phenyl)-1H-[1,2,4]triazol-3-yl]-pyridine was prepared from 15 N-amino-3-bromo-pyridine-2-carboxamidine T12 and 4-isopropyl-benzaldehyde using the protocol described in Example 36. [M+H]⁺ 343.2.

Compound 483: 3-Bromo-2-(5-naphthalen-1-yl-1H-[1,2,4]triazol-3-yl)-pyridine (Example 177)

3-Bromo-2-(5-naphthalen-1-yl-1H-[1,2,4]triazol-3-yl)-pyridine was prepared from N-amino-3-bromo-pyridine-2-carboxamidine and naphthalene-1-carbaldehyde using the protocol described in Example 36. [M+H]⁺ 352.2.

Compound 484: 2-[5-(3-Bromo-pyridin-2-yl)-2H-[1,2,4]triazol-3-yl]-4-tert-butyl-phenol (Example 178)

2-[5-(3-Bromo-pyridin-2-yl)-2H-[1,2,4]triazol-3-yl]-4-tert-butyl-phenol was prepared from N-amino-3-bromo-pyridine-2-carboxamidine and 5-tert-Butyl-2-hydroxy-benzaldehyde using the protocol described in Example 36. [M+H]⁺ 374.3.

Compound 485: 2-[5-(3-Bromo-pyridin-2-yl)-2H-[1,2,4]triazol-3-yl]-4-Nitro-phenol (Example 179)

2-[5-(3-Bromo-pyridin-2-yl)-2H-[1,2,4]triazol-3-yl]-4-Nitro-phenol was prepared from N-amino-3-bromo-pyridine-2-carboxamidine and 5-nitro-2-hydroxy-benzaldehyde using the protocol described in Example 36. [M+H]⁺ 363.1.

Compound 486: 2-[5-(3-Bromo-pyridin-2-yl)-2H-[1,2,4]triazol-3-yl]-4-Fluoro-phenol (Example 180)

2-[5-(3-Bromo-pyridin-2-yl)-2H-[1,2,4]triazol-3-yl]-4-Fluoro-phenol was prepared from N-amino-3-bromo-pyridine-2-carboxamidine and 5-fluoro-2-hydroxy-benzaldehyde using the protocol described in Example 36. [M+H]⁺ 336.1.

Compound 487: 2-[5-(3-Bromo-pyridin-2-yl)-2H-[1,2,4]triazol-3-yl]-4-Methoxy-phenol (Example 181)

2-[5-(3-Bromo-pyridin-2-yl)-2H-[1,2,4]triazol-3-yl]-4-Methoxy-phenol was prepared from N-amino-3-bromo-pyridine-2-carboxamidine and 5-methoxy-2-hydroxy-benzaldehyde using the protocol described in Example 36. [M+H]⁺ 348.3.

Compound 488: 2-[5-(3-Bromo-pyridin-2-yl)-2H-[1,2,4]triazol-3-yl]-4-Chloro-phenol (Example 182)

2-[5-(3-Bromo-pyridin-2-yl)-2H-[1,2,4]triazol-3-yl]-4-Chloro-phenol was prepared from N-amino-3-bromo-pyridine-2-carboxamidine and 5-chloro-2-hydroxy-benzaldehyde using the protocol described in Example 36. [M+H]⁺ 352.3.

Compound 489: 2-[5-(3-Bromo-pyridin-2-yl)-2H-[1,2,4]triazol-3-yl]-6-Ethoxy-phenol (Example 183)

2-[5-(3-Bromo-pyridin-2-yl)-2H-[1,2,4]triazol-3-yl]-6-Ethoxy-phenol was prepared from N-amino-3-bromo-pyridine-2-carboxamidine and 3-ethoxy-2-hydroxy-benzaldehyde using the protocol described in Example 36. [M+H]⁺ 362.2.

Compound 490: 2-[5-(3-Bromo-pyridin-2-yl)-2H-[1,2,4]triazol-3-yl]-5-Benzyloxy-phenol (Example 184)

2-[5-(3-Bromo-pyridin-2-yl)-2H-[1,2,4]triazol-3-yl]-5-Benzyloxy-phenol was prepared from N-amino-3-bromo-pyridine-2-carboxamidine and 4-benzyloxy-2-hydroxy-benzaldehyde using the protocol described in Example 36. [M+H]⁺ 424.2.

Compound 491: 3-Bromo-2-[5-(2-Fluoro-biphenyl-4-yl)-1H-[1,2,4]triazol-3-yl]-pyridine (Example 185)

3-Bromo-2-[5-(2-Fluoro-biphenyl-4-yl)-1H-[1,2,4]triazol-3-yl]-pyridine was prepared from N-amino-3-bromo-pyridine-2-carboxamidine and 2-fluoro-biphenyl-4-carbaldehyde using the protocol described in Example 36. [M+H]⁺ 396.2.

Compound 492: 2-[5-(3-Bromo-pyridin-2-yl)-2H-[1,2,4]triazol-3-yl]-4-Bromo-phenol (Example 186)

2-[5-(3-Bromo-pyridin-2-yl)-2H-[1,2,4]triazol-3-yl]-4-Bromo-phenol was prepared from N-amino-3-bromo-pyridine-2-carboxamidine and 5-bromo-2-hydroxy-benzaldehyde using the protocol described in Example 36. [M+H]⁺ 397.1.

Compound 493: 2-[5-(3-Bromo-pyridin-2-yl)-2H-[1,2,4]triazol-3-yl]-4-Methyl-phenol (Example 187)

2-[5-(3-Bromo-pyridin-2-yl)-2H-[1,2,4]triazol-3-yl]-4-Methyl-phenol was prepared from N-amino-3-bromo-pyridine-2-carboxamidine and 5-methyl-2-hydroxy-benzaldehyde using the protocol described in Example 36. [M+H]⁺ 332.2.

Compound 494: 2-[5-(3-Bromo-pyridin-2-yl)-2H-[1,2,4]triazol-3-yl]-5-Methoxy-phenol (Example 188)

2-[5-(3-Bromo-pyridin-2-yl)-2H-[1,2,4]triazol-3-yl]-5-Methoxy-phenol was prepared from N-amino-3-bromo-pyridine-2-carboxamidine and 4-methoxy-2-hydroxy-benzaldehyde using the protocol described in Example 36. [M+H]⁺ 348.3.

Compound 495: 2-[5-(3-Bromo-pyridin-2-yl)-2H-[1,2,4]triazol-3-yl]-phenol (Example 189)

2-(5-(3-Bromo-pyridin-2-yl)-2H-[1,2,4]triazol-3-yl)-phenol was prepared from N-amino-3-bromo-pyridine-2-carboxamidine and 2-hydroxy-benzaldehyde using the protocol described in Example 36. [M+H]⁺ 318.2.

Compound 496: 2-[5-(3-Bromo-pyridin-2-yl)-2H-[1,2,4]triazol-3-yl]-6-Methoxy-phenol (Example 190)

2-[5-(3-Bromo-pyridin-2-yl)-2H-[1,2,4]triazol-3-yl]-6-Methoxy-phenol was prepared from N-amino-3-bromo-pyridine-2-carboxamidine and 3-methoxy-2-hydroxy-benzaldehyde using the protocol described in Example 36. [M+H]⁺ 348.3.

Compound 497: 3-Bromo -2-[5-(3-nitro-phenyl)-1H-[1,2,4]triazol-3-yl]-pyridine (Example 191)

3-Bromo-2-[5-(3-nitro-phenyl)-1H-[1,2,4]triazol-3-yl]-pyridine was prepared from N-amino-3-bromo-pyridine-2-carboxamidine and 3-nitro-benzaldehyde using the protocol described in Example 36. [M+H]⁺ 347.3.

Compound 498: 2-[5-(3-Bromo-pyridin-2-yl)-2H-[1,2,4]triazol-3-yl]-5-Diethylamino-phenol (Example 192)

2-[5-(3-Bromo-pyridin-2-yl)-2H-[1,2,4]triazol-3-yl]-5-Diethylamino-phenol was prepared from N-amino-3-bromo-pyridine-2-carboxamidine and 4-diethylamino-2-hydroxy-benzaldehyde using the protocol described in Example 36. [M+H]⁺ 389.2.

Compound 499: 2-[5-(3-Bromo-pyridin-2-yl)-2H-[1,2,4]triazol-3-yl]-4-Hydroxy-phenol (Example 193)

2-[5-(3-Bromo-pyridin-2-yl)-2H-[1,2,4]triazol-3-yl]-4-Hydroxy-phenol was prepared from N-amino-3-bromo-pyridine-2-carboxamidine and 2,5-dihydroxybenzaldehyde using the protocol described in Example 36. [M+H]⁺ 334.2.

Compound 500: 3-Bromo-2-[5-(3-methoxy-phenyl)-1H-t1,2,4]triazol-3-yl]-pyridine (Example 500)

3-Bromo 2-[5-(3-Methoxy-phenyl)-1H-[1,2,4]triazol-3-yl]-pyridine was prepared from N-amino-3-bromo-pyridine-2-carboxamidine and 3-methoxy-benzaldehyde using the protocol described in Example 36. [M+H]⁺ 332.3.

Compound 501: 3-Bromo-2-[5-(2-fluoro-5-methoxy-phenyl)-1H-[1 ,2,4]triazol-3-yl]-pyridine (Example 195)

3-Bromo-2-[5-(2-Fluoro-5-methoxy-phenyl)-1H-[1,2,4]triazol-3-yl]-pyridine was prepared from N-amino-3-bromo-pyridine-2-carboxamidine and 2-fluoro-5-methoxy-benzaldehyde using the protocol described in Example 36. [M+H]⁺ 350.3.

Compound 503: 1-(5-(3-Bromo-pyridin-2-yl)-2H-1,2,4]triazol-3-yl)-naphthalen-2-ol (Example 196)

1-(5-(3-Bromo-pyridin-2-yl)-2H-[1,2,4]triazol-3-yl)-naphthalen-2-ol was prepared from N-amino-3-bromo-pyridine-2-carboxamidine and 2-hydroxy-naphthalene-1-carbaldehyde using the protocol described in Example 36. [M+H]⁺ 368.2.

Compound 567: 4-Methyl-2-(5-pyrimidin-2-yl-2H-[1,2,4]triazol-3-yl)-phenol (Example 197)

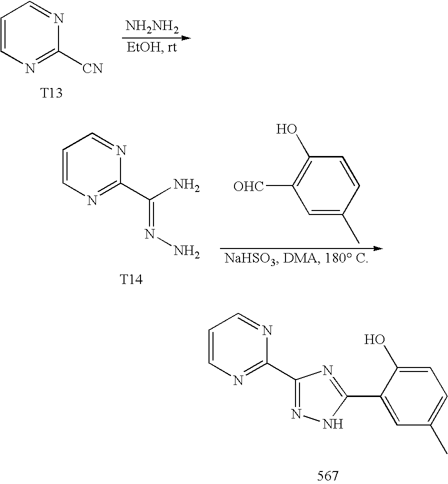

Step 1: Preparation of N-Amino-pyrimidine-2-carboxamidine

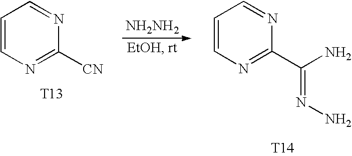

N-Amino-pyrimidine-2-carboxamidine T14 was prepared from pyrimidine-2-carbonitrile T13 and hydrazine using the protocol described in Example 1, step 1. [M+H]⁺ 138.2; 1H NMR (400 MHz, DMSO-D6) δ ppm 5.50 (s, 2 H) 5.61 (s, 2 H) 7.37 (t, J=4.77 Hz, 1 H) 8.74 (d, J=4.77 Hz, 2 H).

Step 2

Compound 567: 4-methyl-2-(5-pyrimidin-2-yl-2H-[1,2,4]triazol-3-yl)-phenol

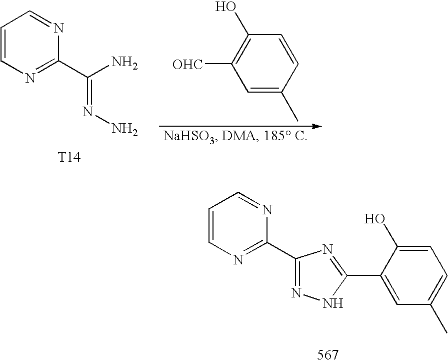

4-Methyl-2-(5-pyrimidin-2-yl-2H-[1,2,4]triazol-3-yl)-phenol was prepared from N-amino-pyrimidine-2-carboxamidine T14 and 2-hydroxy-5-methyl-benzaldehyde using the protocol described in Example 36. [M+H]⁺ 254.2.

Compound 559: 4-tert-Butyl-2-(5-pyrimidin-2-yl-2H-[1,2,4]triazol-3-yl)-phenol (Example 198)

4-tert-Butyl-2-(5-pyrimidin-2-yl-2H-[1,2,4]triazol-3-yl)-phenol was prepared from N-amino-pyrimidine-2-carboxamidine and 5-tert-Butyl-2-hydroxy-benzaldehyde using the protocol described in Example 36. [M+H]⁺ 296.3.

Compound 560: 4-Nitro-2-(5-pyrimidin-2-yl-2H-[1,2,4]triazol-3-yl)-phenol (Example 199)

4-Nitro-2-(5-pyrimidin-2-yl-2H-[1,2,4]triazol-3-yl)-phenol was prepared from N-amino-pyrimidine-2-carboxamidine and 5-nitro-2-hydroxy-benzaldehyde using the protocol described in Example 36. [M+H]⁺ 285.1.

Compound 561: 4-Fluoro-2-(5-pyrimidin-2-yl-2H-[1,2,4]triazol-3-yl)-phenol (Example 200)

4-Fluoro-2-(5-pyrimidin-2-yl-2H-[1,2,4]triazol-3-yl)-phenol was prepared from N-amino-pyrimidine-2-carboxamidine and 5-fluoro-2-hydroxy-benzaldehyde using the protocol described in Example 36. [M+H]⁺ 258.1.

Compound 562: 4-Methoxy-2-(5-pyrimidin-2-yl-2H-[1,2,4]triazol-3-yl)-phenol (Example 201)

4-Methoxy-2-(5-pyrimidin-2-yl-2H-[1,2,4]triazol-3-yl)-phenol was prepared from N-amino-pyrimidine-2-carboxamidine and 5-methoxy-2-hydroxy-benzaldehyde using the protocol described in Example 36. [M+H]⁺ 270.3.

Compound 563: 4-Chloro-2-(5-pyrimidin-2-yl-2H-[1,2,4]triazol-3-yl)-phenol (Example 202)

4-Chloro-2-(5-pyrimidin-2-yl-2H-[1,2,4]triazol-3-yl)-phenol was prepared from N-amino-pyrimidine-2-carboxamidine and 5-chloro-2-hydroxy-benzaldehyde using the protocol described in Example 36. [M+H]⁺ 274.3.

Compound 564: 6-Ethoxy-2-(5-pyrimidin-2-yl-2H-[1,2,4]triazol-3-yl)-phenol (Example 203)

6-Ethoxy-2-(5-pyrimidin-2-yl-2H-[1,2,4]triazol-3-yl)-phenol was prepared from N-amino-pyrimidine-2-carboxamidine and 3-ethoxy-2-hydroxy-benzaldehyde using the protocol described in Example 36. [M+H]⁺ 284.2.

Compound 565: 5-Benzyloxy-2-(5-pyrimidin-2-yl-2H-[1,2,4]triazol-3-yl)-phenol (Example 204)

5-Benzyloxy-2-(5-pyrimidin-2-yl-2H-[1,2,4]triazol-3-yl)-phenol was prepared from N-amino-pyrimidine-2-carboxamidine and 4-benzyloxy-2-hydroxy-benzaldehyde using the protocol described in Example 36. [M+H]⁺ 346.2.

Compound 566: 4-Bromo-2-(5-pyrimidin-2-yl-2H-[1,2,4]triazol-3-yl)-phenol (Example 205)

4-Bromo-2-(5-pyrimidin-2-yl-2H-[1,2,4]triazol-3-yl)-phenol was prepared from N-amino-pyrimidine-2-carboxamidine and 5-bromo-2-hydroxy-benzaldehyde using the protocol described in Example 36. [M+H]⁺ 319.1.

Compound 568: 5-Methoxy-2-(5-pyrimidin-2-yl-2H-[1,2,4]triazol-3-yl)-phenol (Example 206)

5-Methoxy-2-(5-pyrimidin-2-yl-2H-[1,2,4]triazol-3-yl)-phenol was prepared from N-amino-pyrimidine-2-carboxamidine and 4-methoxy-2-hydroxy-benzaldehyde using the protocol described in Example 36. [M+H]⁺ 270.3.

Compound 569: 2-(5-pyrimidin-2-yl-2H-[1,2,4]triazol-3-yl)-phenol (Example 207)

2-(5-pyrimidin-2-yl-2H-[1,2,4]triazol-3-yl)-phenol was prepared from N-amino-pyrimidine-2-carboxamidine and 2-hydroxy-benzaldehyde using the protocol described in Example 36. [M+H]⁺ 240.2.

Compound 570: 6-Methoxy-2-(5-pyrimidin-2-yl-2H-[1,2,4]triazol-3-yl)-phenol (Example 208)

6-Methoxy-2-(5-pyrimidin-2-yl-2H-[1,2,4]triazol-3-yl)-phenol was prepared from N-amino-pyrimidine-2-carboxamidine and 3-methoxy-2-hydroxy-benzaldehyde using the protocol described in Example 36. [M+H]⁺ 270.3.

Compound 571: 5-Diethylamino-2-(5-pyrimidin-2-yl-2H-[1,2,4]triazol-3-yl)-phenol (Example 209)

5-Diethylamino-2-(5-pyrimidin-2-yl-2H-[1,2,4]triazol-3-yl)-phenol was prepared from N-amino-pyrimidine-2-carboxamidine and 4-diethylamino-2-hydroxy-benzaldehyde using the protocol described in Example 36. [M+H]⁺ 311.2.

Compound 572: 4-Hydroxy-2-(5-pyrimidin-2-yl-2H-[1,2,4]triazol-3-yl)-phenol (Example 210)

4-Hydroxy-2-(5-pyrimidin-2-yl-2H-[1,2,4]triazol-3-yl)-phenol was prepared from N-amino-pyrimidine-2-carboxamidine and 2,5-dihydroxybenzaldehyde using the protocol described in Example 36. [M+H]⁺ 256.2.

Compound 573: 1-(5-pyrimidin-2-yl-2H-[1,2,4]triazol-3-yl)-naphthalen-2-ol (Example 211)

1-(5-pyrimidin-2-yl-2H-[1,2,4]triazol-3-yl)-naphthalen-2-ol was prepared from N-amino-pyrimidine-2-carboxamidine and 2-hydroxy-naphthalene-1-carbaldehyde using the protocol described in Example 36. [M+H]⁺ 290.2.

Compound 574: 2-(5-Pyrimidin-2-yl-2H-[1,2,4]triazol-3-yl)-naphthalen-1-ol (Example 212)

2-(5-Pyrimidin-2-yl-2H-[1,2,4]triazol-3-yl)-naphthalen-1-olwas prepared from N-amino-pyrimidine-2-carboxamidine and 1-hydroxy-naphthalene-2-carbaldehyde using the protocol described in Example 36. [M+H]⁺ 290.2.

Compound 583: 4-Methyl-2-(5-pyrazin-2-yl-2H-[1,2,4]triazol-3-yl)-phenol (Example 213)

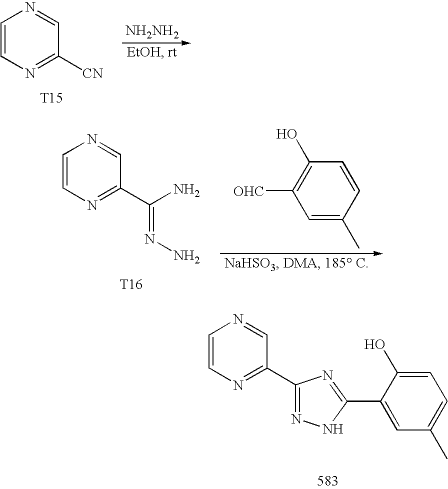

Step 1: Preparation of N-Amino-pyrazine-2-carboxamidine

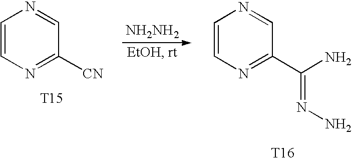

N-Amino-pyrazine-2-carboxamidine T16 was prepared from pyrazine-2-carbonitrile T15 and hydrazine using the protocol described in Example 1, step 1. [M+H]⁺ 138.2.

Step 2

Compound 583: 4-methyl-2-(5-pyrazin-2-yl-2H-[1,2,4]triazol-3-yl)-phenol

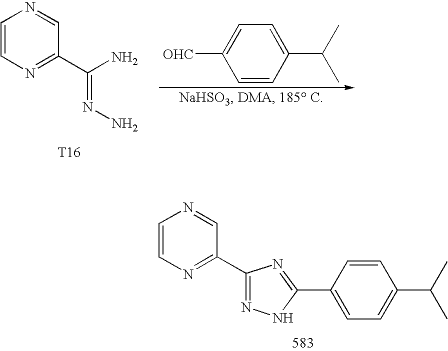

4-Methyl-2-(5-pyrazin-2-yl-2H-[1,2,4]triazol-3-yl)-phenol was prepared from N-amino-pyrazine-2-carboxamidine T16 and 2-hydroxy-5-methyl-benzaldehyde using the protocol described in Example 36. [M+H]⁺ 254.2.

Compound 575: 4-tert-Butyl-2-(5-pyrazin-2-yl-2H-[1,2,4]triazol-3-yl)-phenol (Example 214)

4-tert-Butyl-2-(5-pyrazin-2-yl-2H-[1,2,4]triazol-3-yl)-phenol was prepared from N-amino-pyrazine-2-carboxamidine and 5-tert-butyl-2-hydroxy-benzaldehyde using the protocol described in Example 36. [M+H]⁺ 296.3.

Compound 576: 4-Nitro-2-(5-pyrazin-2-yl-2H-[1,2,4]triazol-3-yl)-phenol (Example 215)

4-Nitro-2-(5-pyrazin-2-yl-2H-[1,2,4]triazol-3-yl)-phenol was prepared from N-amino-pyrazine-2-carboxamidine and 5-nitro-2-hydroxy-benzaldehyde using the protocol described in Example 36. [M+H]⁺ 285.1.

Compound 577: 4-Fluoro-2-(5-pyrazin-2-yl-2H-[1,2,4]triazol-3-yl)-phenol (Example 216)

4-Fluoro-2-(5-pyrazin-2-yl-2H-[1,2,4]triazol-3-yl)-phenol was prepared from N-amino-pyrazine-2-carboxamidine and 5-fluoro-2-hydroxy-benzaldehyde using the protocol described in Example 36. [M+H]⁺ 258.1.

Compound 578: 4-Methoxy-2-(5-pyrazin-2-yl-2H-[1,2,4]triazol-3-yl)-phenol (Example 217)

4-Methoxy-2-(5-pyrazin-2-yl-2H-[1,2,4]triazol-3-yl)-phenol was prepared from N-amino-pyrazine-2-carboxamidine and 5-methoxy-2-hydroxy-benzaldehyde using the protocol described in Example 36. [M+H]⁺ 270.3.

Compound 579: 4-Chloro-2-(5-pyrazin-2-yl-2H-[1,2,4]triazol-3-yl)-phenol (Example 218)

4-Chloro-2-(5-pyrazin-2-yl-2H-[1,2,4]triazol-3-yl)-phenol was prepared from N-amino-pyrazine-2-carboxamidine and 5-chloro-2-hydroxy-benzaldehyde using the protocol described in Example 36. [M+H]⁺ 274.3.

Compound 580: 6-Ethoxy-2-(5-pyrazin-2-yl-2H-[1,2,4]triazol-3-yl)-phenol (Example 219)

6-Ethoxy-2-(5-pyrazin-2-yl-2H-[1,2,4]triazol-3-yl)-phenol was prepared from N-amino-pyrazine-2-carboxamidine and 3-ethoxy-2-hydroxy-benzaldehyde using the protocol described in Example 36. [M+H]⁺ 284.2.

Compound 581: 5-Benzyloxy-2-(5-pyrazin-2-yl-2H-[1,2,4]triazol-3-yl)-phenol (Example 220)

5-Benzyloxy-2-(5-pyrazin-2-yl-2H-[1,2,4]triazol-3-yl)-phenol was prepared from N-amino-pyrazine-2-carboxamidine and 4-benzyloxy-2-hydroxy-benzaldehyde using the protocol described in Example 36. [M+H]⁺ 346.2.

Compound 582: 4-Bromo-2-(5-pyrazin-2-yl-2H-[1,2,4]triazol-3-yl)-phenol (Example 221)

4-Bromo-2-(5-pyrazin-2-yl-2H-[1,2,4]triazol-3-yl)-phenol was prepared from N-amino-pyrazine-2-carboxamidine and 5-bromo-2-hydroxy-benzaldehyde using the protocol described in Example 36. [M+H]⁺ 319.1.

Example 584: 5-Methoxy-2-(5-pyrazin-2-yl-2H-[1,2,4]triazol-3-yl)-phenol (Example 222)

5-Methoxy-2-(5-pyrazin-2-yl-2H-[1,2,4]triazol-3-yl)-phenol was prepared from N-amino-pyrazine-2-carboxamidine and 4-methoxy-2-hydroxy-benzaldehyde using the protocol described in Example 36. [M+H]⁺ 270.3.

Compound 585: 2-(5-pyrazin-2-yl-2H-[1,2,4]triazol-3-yl)-phenol (Example 223)

2-(5-pyrazin-2-yl-2H-[1,2,4]triazol-3-yl)-phenol was prepared from N-amino-pyrazine-2-carboxamidine and 2-hydroxy-benzaldehyde using the protocol described in Example 36. [M+H]⁺ 240.2.

Compound 586: 6-Methoxy-2-(5-pyrazin-2-yl-2H-[1,2,4]triazol-3-yl)-phenol (Example 224)

6-Methoxy-2-(5-pyrazin-2-yl-2H-[1,2,4]triazol-3-yl)-phenol was prepared from N-amino-pyrazine-2-carboxamidine and 3-methoxy-2-hydroxy-benzaldehyde using the protocol described in Example 36. [M+H]⁺ 270.3.

Compound 587: 5-Diethylamino-2-(5-pyrazin-2-yl-2H-[1,2,4]triazol-3-yl)-phenol (Example 225)

5-Diethylamino-2-(5-pyrazin-2-yl-2H-[1,2,4]triazol-3-yl)-phenol was prepared from N-amino-pyrazine-2-carboxamidine and 4-diethylamino-2-hydroxy-benzaldehyde using the protocol described in Example 36. [M+H]⁺ 311.2.

Compound 588: 4-Hydroxy-2-(5-pyrazin-2-yl-2H-[1,2,4]triazol-3-yl)-phenol (Example 226)

4-Hydroxy-2-(5-pyrazin-2-yl-2H-[1,2,4]triazol-3-yl)-phenol was prepared from N-amino-pyrazine-2-carboxamidine and 2,5-dihydroxybenzaldehyde using the protocol described in Example 36. [M+H]⁺ 256.2.

Compound 589: 1-(5-pyrazin-2-yl-2H-[1,2,4]triazol-3-yl)-naphthalen-2-ol (Example 227)

1-(5-pyrazin-2-yl-2H-[1,2,4]triazol-3-yl)-naphthalen-2-ol was prepared from N-amino-pyrazine-2-carboxamidine and 2-hydroxy-naphthalene-1-carbaldehyde using the protocol described in Example 36. [M+H]⁺ 290.2.

Compound 591: 2-(5-pyrazin-2-yl-2H-[1,2,4]triazol-3-yl)-naphthalen-1-ol (Example 228)

2-(5-pyrazin-2-yl-2H-[1,2,4]triazol-3-yl)-naphthalen-1-olwas prepared from N-amino-pyrazine-2-carboxamidine and 1-hydroxy-naphthalene-2-carbaldehyde using the protocol described in Example 36. [M+H]⁺ 290.2.

Compound 590: 2,4-Dibromo-6-(5-pyrazin-2-yl-2H-[1,2,4]triazol-3-yl)-phenol (Example 229)

2,4-Dibromo-6-(5-pyrazin-2-yl-2H-[1,2,4]triazol-3-yl)-phenol was prepared from N-amino-pyrazine-2-carboxamidine and 3,5-dibromo-2-hydroxy-carbaldehyde using the protocol described in Example 36. [M+H]⁺ 398.2.

Compound 540: 4-Methyl-2-[5-(3-trifluoromethyl-pyridin-2-yl)-2H-[1,2,4]triazol-3-yl]-phenol (Example 230)

Compound 516: 4-Methyl-2-[5-(5-trifluoromethyl-pyridin-2-yl)-2H-[1,2,4]triazol-3-yl]-phenol (Example 231)

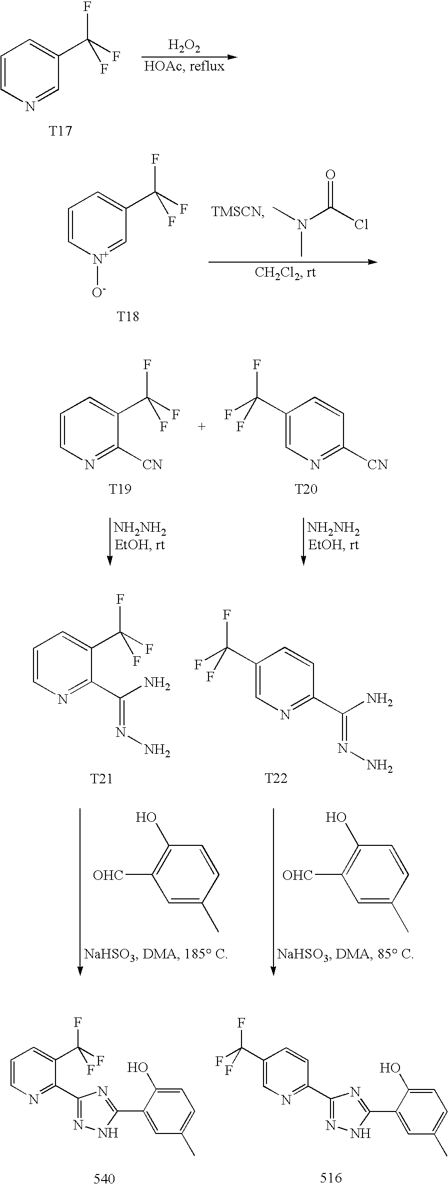

Step 1: Preparation of 3-trifluoromethyl-pyndine 1-oxide

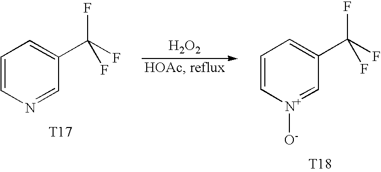

To a solution of 3-trifluoromethylpyridine T17 ( 4.44 g, 30 mmol) in glacial acetic acid (20 mL) was added 30% hydrogen peroxide (3.4 mL, 30 mmol). The reaction mixture was refluxed for 24 h. After removal of the solvent, the residue was repartitioned between ethyl acetate and water. The aqueous layer was separated and extracted with ethyl acetate four times. The combined organic phase was washed with brine, dried over anhydrous magnesium sulfate and concentrated in vacuo. The resulting solid T18 (4.50 g, 91%) was used without further purification. 1H NMR (400 MHz, CHLOROFORM-D) δ ppm 7.37-7.52 (m, 2 H) 8.36 (d, J=6.23 Hz, 1 H) 8.48 (s, 1 H).

Step 2: Preparation of 3-trifluoromethyl-pyridine-2-carbonitrile and 5-trifluoromethyl-pyridine-2-carbonitrile

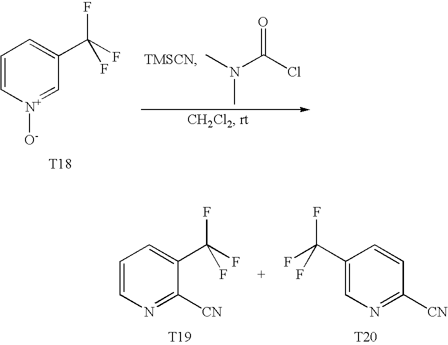

To a solution of 3-trifluoromethyl-pyridine 1-oxide T18 (4.92 g, 30 mmol) and trimethylsilyl cyanide (3.87 g, 39 mmol) in dichloromethane (30 mL) was added dimethylcarbamyl chloride (4.20 g, 39 mmol) in dichloromethane (5 mL) dropwise. The reaction mixture was stirred at room temperature for 48 h. A solution of 10% aqueous potassium carbonate (30 mL) was added. The aqueous layer was separated and extracted with dichloromethane. The combined organic layers were washed with brine and dried over anhydrous MgSO4.

After removal of the solvent, the residue was purified by column chromatography (10-20% EtOAc-Hexane) to give 3-rifluoromethyl-pyridine-2-carbonitrile T19 (1.92 g, 37%) and 5-tribluoromethyl-pyridine-2-carbonitrile T20 (2.83 g, 55%). 3-rifluoromethyl-pyridine-2-carbonitrile: 1H NMR (400 MHz, CHLOROFORM-D) δ ppm 7.69 (dd, J=8.07, 4.77 Hz, 1 H) 8.12 (d, J=7.33 Hz, 1 H) 8.90 (d, J=4.77 Hz, 1 H); 5-rifluoromethyl-pyridine-2-carbonitrile: 1H NMR (400 MHz, CHLOROFORM-D) δ ppm 7.85 (d, J=8.07 Hz, 1 H) 8.11 (dd, J=8.07, 2.20 Hz, 1 H) 8.98 (s, 1 H).

Step 3: Preparation of N-Amino3-trifluoromethyl-piridine-2-carboxamidine and N-Amino-5-trifluoromethyl-piridine-2-carboxamidine

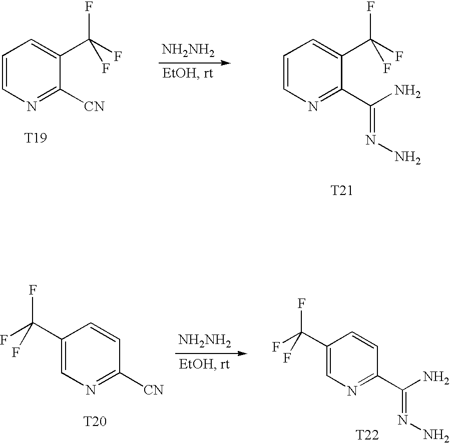

A mixture of 3-trifluoromethyl-pyridine-2-carbonitrile T19 (346 mg g, 2.0 mmol) and hydrazine hydrate (2.0 g, 40.0 mmol) in ethanol (2 mL) was stirred at room temperature for 24 hr. After removal of the solvent, the residue was partitioned between water and ethyl acetate. The aqueous layer was separated and extracted with ethyl acetate. The combined organic layers were washed with brine, dried over anhydrous magnesium sulfate and evaporated to dryness to yield 3-trifluoromethyl-pyridine-2-carbonitrile T21 (306 mg g, 75%). [M+H]⁺ 205.2.

A mixture of 5-trifluoromethyl-pyridine-2-carbonitrile T20 (519 mg g, 3.0 mmol) and hydrazine hydrate (3.1 g, 30.0 mmol) in ethanol (3 mL) was stirred at room temperature for 4 hr. After removal of the solvent, the residue was partitioned between water and ethyl acetate. The aqueous layer was separated and extracted with ethyl acetate. The combined organic layers were washed with brine, dried over anhydrous magnesium sulfate and evaporated to dryness to yield 5-trifluoromethyl-pyridine-2-carbonitrile T22 (490 mg g, 80%). [M+H]⁺ 205.2; 1H NMR (400 MHz, DMSO-D6) δ ppm 5.62 (s, 2 H) 5.77 (s, 2 H) 8.06 (d, J=1.83 Hz, 2 H) 8.82 (s, 1 H).

Step 4

Compound 540: 4-Methyl-2-[5-(3-trifluoromethyl-pyridin-2-yl)-2H-[1,2,4]triazol-3-yl]-phenol

Compound 516: 4-Methyl-2-[5-(5-trifluoromethyl-pyridin-2-yl)-2H-[1,2,4]triazol-3-yl]-phenol

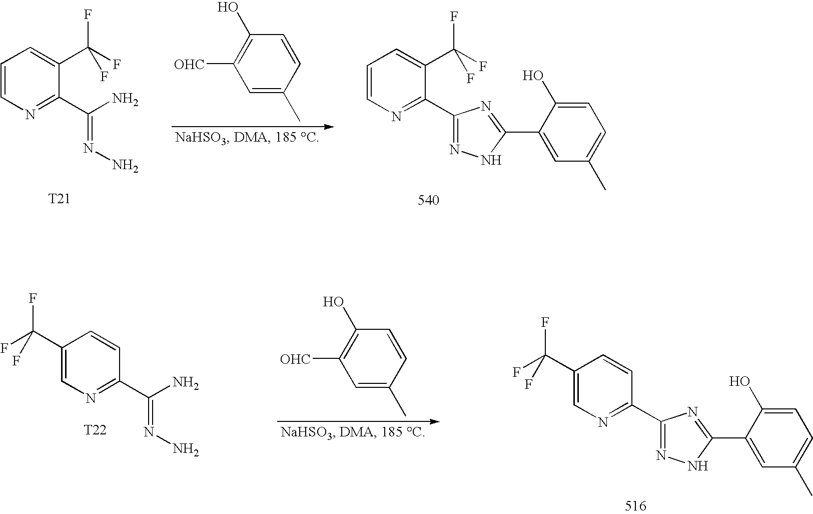

4-Methyl-2-[5-(3-trifluoromethyl-pyridin-2-yl)-2H-[1,2,4]triazol-3-yl]-phenol was prepared from N-amino-3-trifluoromethyl-pyridine-2-carboxamidine and 5-methyl-2-hydroxy-benzaldehyde using the protocol described in Example 36. [M+H]⁺ 321.2.

4-Methyl-2-[5-(5-trifluoromethyl-pyridin-2-yl)-2H-[1,2,4]triazol-3-yl]-phenol was prepared from N-amino-3-trifluoromethyl-pyridine-2-carboxamidine and 5-methyl-2-hydroxy-benzaldehyde using the protocol described in Example 36. [M+H]⁺ 321.2.

Compound 530: 2-(5-Naphthalen-1-yl-1H-[1,2,4]triazol-3-yl)-3-trifluoromethyl-pyridine (Example 232)

2-(5-Naphthalen-1-yl-1H-[1,2,4]triazol-3-yl)-3-trifluoromethyl-pyridine was prepared from N-amino-3-trifluoromethyl-pyridine-2-carboxamidine and naphthalene-1-carbaldehyde using the protocol described in Example 36. [M+H]⁺ 341.2.

Compound 531: 4-tert-Butyl-2-[5-(3-trifluoromethyl-pyridin-2-yl)-2H-[1,2,4]triazol-3-yl]-phenol (Example 233)

4-tert-Butyl-2-[5-(3-trifluoromethyl-pyridin-2-yl)-2H-[1,2,4]triazol-3-yl]-phenol was prepared from N-amino-3-trifluoromethyl-pyridine-2-carboxamidine and 5-tert-Butyl-2-hydroxy-benzaldehyde using the protocol described in Example 36. [M+H]⁺ 363.3.

Compound 532: 4-Nitro-2-[5-(3-trifluoromethyl-pyridin-2-yl)-2H-[1,2,4]triazol-3-yl]-phenol (Example 234)

4-Nitro-2-[5-(3-trifluoromethyl-pyridin-2-yl)-2H-[1,2,4]triazol-3-yl]-phenol was prepared from N-amino-3-trifluoromethyl-pyridine-2-carboxamidine and 5-nitro-2-hydroxy-benzaldehyde using the protocol described in Example 36. [M+H]⁺ 352.1.

Compound 533: 4-Fluoro-2-[5-(3-trifluoromethyl-pyridin-2-yl)-2H-[1,2,4]triazol-3-yl]-phenol (Example 235)

4-Fluoro-2-[5-(3-trifluoromethyl-pyridin-2-yl)-2H-[1,2,4]triazol-3-yl]-phenol was prepared from N-amino-3-trifluoromethyl-pyridine-2-carboxamidine and 5-fluoro-2-hydroxy-benzaldehyde using the protocol described in Example 36. [M+H]⁺ 325.1.

Compound 534: 4-Methoxy-2-[5-(3-trifluoromethyl-pyridin-2-yl)-2H-[1,2,4]triazol-3-yl]-phenol (Example 236)

4-Methoxy-2-[5-(3-trifluoromethyl-pyridin-2-yl)-2H-[1,2,4]triazol-3-yl]-phenol was prepared from N-amino-3-trifluoromethyl-pyridine-2-carboxamidine and 5-methoxy-2-hydroxy-benzaldehyde using the protocol described in Example 36. [M+H]⁺ 337.3.

Compound 535: 4-Chloro-2-[5-(3-trifluoromethyl-pyridin-2-yl)-2H-[1,2,4]triazol-3-yl]-phenol (Example 237)

4-Chloro-2-[5-(3-trifluoromethyl-pyridin-2-yl)-2H-[1,2,4]triazol-3-yl]-phenol was prepared from N-amino-3-trifluoromethyl-pyridine-2-carboxamidine and 5-chloro-2-hydroxy-benzaldehyde using the protocol described in Example 36. [M+H]⁺ 341.3.

Compound 536: 6-Ethoxy-2-[5-(3-trifluoromethyl-pyridin-2-yl)-2H-[1,2,4]triazol-3-yl]-phenol (Example 238)

6-Ethoxy-2-[5-(3-trifluoromethyl-pyridin-2-yl)-2H-[1,2,4]triazol-3-yl]-phenol was prepared from N-amino-3-trifluoromethyl-pyridine-2-carboxamidine and 3-ethoxy-2-hydroxy-benzaldehyde using the protocol described in Example 36. [M+H]⁺ 351.2.

Compound 537: 5-Benzyloxy-2-[5-(3-trifluoromethyl-pyridin-2-yl)-2H-[1,2,4]triazol-3-yl]-phenol (Example 239)

5-Benzyloxy-2-[5-(3-trifluoromethyl-pyridin-2-yl)-2H-[1,2,4]triazol-3-yl]-phenol was prepared from N-amino-3-trifluoromethyl-pyridine-2-carboxamidine and 4-benzyloxy-2-hydroxy-benzaldehyde using the protocol described in Example 36. [M+H]⁺ 413.2.

Compound 538: 2-[5-(2-Fluoro-biphenyl-4-yl)-1H-[1,2,4]triazol-3-yl]-3-trifluoromethyl-pyridine (Example 240)

2-[5-(2-Fluoro-biphenyl-4-yl)-1H-[1,2,4]triazol-3-yl]-3-trifluoromethyl-pyridine was prepared from N-amino-3-trifluoromethyl-pyridine-2-carboxamidine and 2-fluoro-biphenyl-4-carbaldehyde using the protocol described in Example 36. [M+H]⁺ 385.2.

Compound 539: 4-Bromo-2-[5-(3-trifluoromethyl-pyridin-2-yl)-2H-[1,2,4]triazol-3-yl]-phenol (Example 241)

4-Bromo-2-[5-(3-trifluoromethyl-pyridin-2-yl)-2H-[1,2,4]triazol-3-yl]-phenol was prepared from N-amino-3-trifluoromethyl-pyridine-2-carboxamidine and 5-bromo-2-hydroxy-benzaldehyde using the protocol described in Example 36. [M+H]⁺ 386.1.

Compound 541: 5-Methoxy-2-[5-(3-trifluoromethyl-pyridin-2-yl)-2H-[1,2,4]triazol-3-yl]-phenol (Example 242)

5-Methoxy-2-[5-(3-trifluoromethyl-pyridin-2-yl)-2H-[1,2,4]triazol-3-yl]-phenol was prepared from N-amino-3-trifluoromethyl-pyridine-2-carboxamidine and 4-methoxy-2-hydroxy-benzaldehyde using the protocol described in Example 36. [M+H]⁺ 337.3.

Compound 542: 2-[5-(3-trifluoromethyl-pyridin-2-yl)-2H-[1,2,4]triazol-3-yl]-phenol (Example 243)

2-[5-(3-trifluoromethyl-pyridin-2-yl)-2H-[1,2,4]triazol-3-yl]-phenol was prepared from N-amino-3-trifluoromethyl-pyridine-2-carboxamidine and 2-hydroxy-benzaldehyde using the protocol described in Example 36. [M+H]⁺ 307.2.

Compound 543: 6-Methoxy-2-[5-(3-trifluoromethyl-pyridin-2-yl)-2H-[1,2,4]triazol-3-yl]-phenol (Example 244)

6-Methoxy-2-[5-(3-trifluoromethyl-pyridin-2-yl)-2H-[1,2,4]triazol-3-yl]-phenol was prepared from N-amino-3-trifluoromethyl-pyridine-2-carboxamidine and 3-methoxy-2-hydroxy-benzaldehyde using the protocol described in Example 36. [M+H]⁺ 337.3.

Compound 544: 2-[5-(3-Nitro-phenyl)-1H-[1,2,4]triazol-3-yl]-3-trifluoromethyl-pyridine (Example 245)

2-[5-(3-Nitro-phenyl)-1H-[1,2,4]triazol-3-yl]-3-trifluoromethyl-pyridine was prepared from N-amino-3-trifluoromethyl-pyridine-2-carboxamidine and 3-nitro-benzaldehyde using the protocol described in Example 36. [M+H]⁺ 336.3.

Compound 545: 5-Diethylamino-2-[5-(3-trifluoromethyl-pyridin-2-yl)-2H-[1,2,4]triazol-3-yl]-phenol (Example 246)

5-Diethylamino-2-[5-(3-trifluoromethyl-pyridin-2-yl)-2H-[1,2,4]triazol-3-yl]-phenol was prepared from N-amino-3-trifluoromethyl-pyridine-2-carboxamidine and 4-diethylamino-2-hydroxy-benzaldehyde using the protocol described in Example 36. [M+H]⁺ 378.2.

Compound 546: 4-Hydroxy-2-[5-(3-trifluoromethyl-pyridin-2-yl)-2H-[1,2,4]triazol-3-yl]-phenol (Example 247)

4-Hydroxy-2-[5-(3-trifluoromethyl-pyridin-2-yl)-2H-[1,2,4]triazol-3-yl]-phenol was prepared from N-amino-3-trifluoromethyl-pyridine-2-carboxamidine and 2,5-dihydroxybenzaldehyde using the protocol described in Example 36. [M+H]⁺ 323.2.

Compound 547: 2-[5-(3-Methoxy-phenyl)-1H-[1,2,4]triazol-3-yl]-3-trifluoromethyl-pyridine (Example 248)

2-[5-(3-Methoxy-phenyl)-1H-[1,2,4]triazol-3-yl]-3-trifluoromethyl-pyridine was prepared from N-amino-3-trifluoromethyl-pyridine-2-carboxamidine and 3-methoxy-benzaldehyde using the protocol described in Example 36. [M+H]⁺ 321.3.

Compound 548: 2-[5-(4-Isopropyl-phenyl)-1H-[1,2,4]triazol-3-yl]-3-trifluoromethyl-pyridine (Example 249)

2-[5-(4-Isopropyl-phenyl)-1H-[1,2,4]triazol-3-yl]-3-trifluoromethyl-pyridine was prepared from N-anino-3-trifluoromethyl-pyridine-2-carboxamidine and 4-isopropyl-benzaldehyde using the protocol described in Example 36. [M+H]⁺ 333.2.

Compound 549: 1-[5-(3-trifluoromethyl-pyridin-2-yl)-2H-[1,2,4]triazol-3-yl]-naphthalen-2-ol (Example 250)

1-[5-(3-trifluoromethyl-pyridin-2-yl)-2H-[1,2,4]triazol-3-yl]-naphthalen-2-ol was prepared from N-amino-3-trifluoromethyl-pyridine-2-carboxamidine and 2-hydroxy-naphthalene-1-carbaldehyde using the protocol described in Example 36. [M+H]⁺ 357.2.

Compound 550: 2-[5-(3-Bromo-phenyl)-1H-[1,2,4]triazol-3-yl]-3-trifluoromethyl-pyridine (Example 251)

2-[5-(3-Bromo-phenyl)-1H-[1,2,4]triazol-3-yl]-3-trifluoromethyl-pyridine was prepared from N-amino-3-trifluoromethyl-pyridine-2-carboxamidine and 3-bromo-benzaldehyde using the protocol described in Example 36. [M+H]⁺ 370.2.

Compound 551: 2-[5-(2,6-Dichloro -phenyl)-1H-[1,2,4]triazol-3-yl]-3-trifluoromethyl-pyridine (Example 252)

2-[5-(2,6-Dichloro-phenyl)-1 H-[1,2,4]triazol-3-yl]-3-trifluoromethyl-pyridine was prepared from N-amino-3-trifluoromethyl-pyridine-2-carboxamidine and 2,6-dichloro-benzaldehyde using the protocol described in Example 36. [M+H]⁺ 360.2.

Compound 552: 2-[5-(4-Bromo-2-fluoro-phenyl)-1H-[1,2,4]triazol-3-yl]-3-trifluoromethyl-pyridine (Example 253)

2-[5-(4-Bromo-2-fluoro-phenyl)-1H-[1,2,4]triazol-3-yl]-3-trifluoromethyl-pyridine was prepared from N-amino-3-trifluoromethyl-pyridine-2-carboxamidine and 4-bromo-2-fluoro-benzaldehyde using the protocol described in Example 36. [M+H]⁺ 388.3.

Compound 553: 2-[5-(5-Bromo-2-methoxy-phenyl)-1H-[1,2,4]triazol-3-yl]-3-trifluoromethyl-pyridine (Example 254)

2-[5-(5-Bromo-2-methoxy-phenyl)-1H-[1,2,4]triazol-3-yl]-3-trifluoromethyl-pyridine was prepared from N-amino-3-trifluoromethyl-pyridine-2-carboxamidine and 5-bromo-2-methoxy-benzaldehyde using the protocol described in Example 36. [M+H]⁺ 400.3.

Compound 554: 2-(5-Naphthalen-1-yl-1H-[1,2,4]triazol-3-yl)-5-trifluoromethyl-pyridine (Example 255)

2-(5-Naphthalen-1-yl-1H-[ 1,2,4]triazol-3-yl)-5-trifluoromethyl-pyridine was prepared from N-amino-5-trifluoromethyl-pyridine-2-carboxamidine and naphthalene-1-carbaldehyde using the protocol described in Example 36. [M+H]⁺ 341.2.

Compound 507: 4-tert-Butyl-2-[5-(5-trifluoromethyl-pyridin-2-yl)-2H-[1,2,4]triazol-3-yl]-phenol (Example 256)

4-tert-Butyl-2-[5-(5-trifluoromethyl-pyridin-2-yl)-2H-[1,2,4]triazol-3-yl]-phenol was prepared from N-amino-5-trifluoromethyl-pyridine-2-carboxamidine and 5-tert-Butyl-2-hydroxy-benzaldehyde using the protocol described in Example 36. [M+H]⁺ 363.3.

Compound 508: 4-Nitro-2-[5-(5-trifluoromethyl-pyridin-2-yl)-2H-[1,2,4]triazol-3-yl]-phenol (Example 257)

4-Nitro-2-[5-(5-trifluoromethyl-pyridin-2-yl)-2H-[1,2,4]triazol-3-yl]-phenol was prepared from N-amino-5-trifluoromethyl-pyridine-2-carboxamidine and 5-nitro-2-hydroxy-benzaldehyde using the protocol described in Example 36. [M+H]⁺ 352.1.

Compound 509: 4-Fluoro-2-[5-(5-trifluoromethyl-pyridin-2-yl)-2H-[1,2,4]triazol-3-yl]-phenol (Example 258)

4-Fluoro-2-[5-(5-trifluoromethyl-pyridin-2-yl)-2H-[1,2,4]triazol-3-yl]-phenol was prepared from N-amino-S-trifluoromethyl-pyridine-2-carboxamidine and 5-fluoro-2-hydroxy-benzaldehyde using the protocol described in Example 36. [M+H]⁺ 325.1.

Compound 510: 4-Methoxy-2-[5-(5-trifluoromethyl-pyridin-2-yl)-2H-[1,2,4]triazol-3-yl]-phenol (Example 259)

4-Methoxy-2-[5-(5-trifluoromethyl-pyridin-2-yl)-2H-[1,2,4]triazol-3-yl]-phenol was prepared from N-amino-5-trifluoromethyl-pyridine-2-carboxamidine and 5-methoxy-2-hydroxy-benzaldehyde using the protocol described in Example 36. [M+H]⁺ 337.3.

Compound 511: 4-Chloro-2-[5-(5-trifluoromethyl-pyridin-2-yl)-2H-[1,2,4]triazol-3-yl]-phenol (Example 260)

4-Chloro-2-[5-(5-trifluoromethyl-pyridin-2-yl)-2H-[1,2,4]triazol-3-yl]-phenol was prepared from N-amino-5-trifluoromethyl-pyridine-2-carboxamidine and 5-chloro-2-hydroxy-benzaldehyde using the protocol described in Example 36. [M+H]⁺ 341.3.

Compound 512: 6-Ethoxy-2-[5-(5-trifluoromethyl-pyridin-2-yl)-2H-[1,2,4]triazol-3-yl]-phenol (Example 261)

6-Ethoxy-2-[5-(5-trifluoromethyl-pyridin-2-yl)-2H-[1,2,4]triazol-3-yl]-phenol was prepared from N-amino-5-trifluoromethyl-pyridine-2-carboxamidine and 3-ethoxy-2-hydroxy-benzaldehyde using the protocol described in Example 36. [M+H]⁺ 351.2.

Compound 513: 5-Benzyloxy-2-[5-(5-trifluoromethyl-pyridin-2-yl)-2H-[1,2,4]triazol-3-yl]-phenol (Example 262)

5-Benzyloxy-2-[5-(5-trifluoromethyl-pyridin-2-yl)-2H-[1,2,4]triazol-3-yl]-phenol was prepared from N-amino-5-trifluoromethyl-pyridine-2-carboxamidine and 4-benzyloxy-2-hydroxy-benzaldehyde using the protocol described in Example 36. [M+H]⁺ 413.2.

Compound 514: 2-[5-(2-Fluoro-biphenyl-4-yl)-1H-[1,2,4]triazol-3-yl]-5-trifluoromethyl-pyridine (Example 263)

2-[5-(2-Fluoro-biphenyl-4-yl)-1H-[1,2,4]triazol-3-yl]-5-trifluoromethyl-pyridine was prepared from N-amino-5-trifluoromethyl-pyridine-2-carboxamidine and 2-fluoro-biphenyl-4-carbaldehyde using the protocol described in Example 36. [M+H]⁺ 385.2.

Compound 515: 4-Bromo-2-[5-(5-trifluoromethyl-pyridin-2-yl)-2H-[1,2,4]triazol-3-yl]-phenol (Example 264)

4-Bromo-2-[5-(5-trifluoromethyl-pyridin-2-yl)-2H-[1,2,4]triazol-3-yl]-phenol was prepared from N-amino-5-trifluoromethyl-pyridine-2-carboxamidine and 5-bromo-2-hydroxy-benzaldehyde using the protocol described in Example 36. [M+H]⁺ 386.1.

Compound 517: 5-Methoxy-2-[5-(5-trifluoromethyl-pyridin-2-yl)-2H-[1,2,4]triazol-3-yl]-phenol (Example 265)

5-Methoxy-2-[5-(5-trifluoromethyl-pyridin-2-yl)-2H-[1,2,4]triazol-3-yl]-phenol was prepared from N-amino-5-trifluoromethyl-pyridine-2-carboxamidine and 4-methoxy-2-hydroxy-benzaldehyde using the protocol described in Example 36. [M+H]⁺ 337.3.

Compound 518: 2-[5-(5-trifluoromethyl-pyridin-2-yl)-2H-[1,2,4]triazol-3-yl]-phenol (Example 266)

2-[5-(5-trifluoromethyl-pyridin-2-yl)-2H-[1,2,4]triazol-3-yl]-phenol was prepared from N-amino-5-trifluoromethyl-pyridine-2-carboxamidine and 2-hydroxy-benzaldehyde using the protocol described in Example 36. [M+H]⁺ 307.2.

Compound 519: 6-Methoxy-2-[5-(5-trifluoromethyl-pyridin-2-yl)-2H-[1,2,4jtriazol-3-yl]-phenol (Example 267)

6-Methoxy-2-[5-(5-trifluoromethyl-pyridin-2-yl)-2H-[1,2,4]triazol-3-yl]-phenol was prepared from N-amino-5-trifluoromethyl-pyridine-2-carboxamidine and 3-methoxy-2-hydroxy-benzaldehyde using the protocol described in Example 36. [M+H]⁺ 337.3.

Compound 520: 2-[5-(3-Nitro-phenyl)-1H-[l,2,4]triazol-3-yl]-5-trifluoromethyl-pyridine (Example 268)

2-[5-(3-Nitro-phenyl)-1H-[1,2,4]triazol-3-yl]-5-trifluoromethyl-pyridine was prepared from N-amino-5-trifluoromethyl-pyridine-2-carboxamidine and 3-nitro-benzaldehyde using the protocol described in Example 36. [M+H]⁺ 336.3.

Compound 521: 5-Diethylamino-2-[5-(5-trifluoromethyl-pyridin-2-yl)-2H-[1,2,4]triazol-3-yl]-phenol (Example 269)

5-Diethylamino-2-[5-(5-trifluoromethyl-pyridin-2-yl)-2H-[1,2,4]triazol-3-yl]-phenol was prepared from N-amino-5-trifluoromethyl-pyridine-2-carboxamidine and 4-diethylamino-2-hydroxy-benzaldehyde using the protocol described in Example 36. [M+H]⁺ 378.2.

Compound 522: 4-Hydroxy-2-[5-(5-trifluoromethyl-pyridin-2-yl)-2H-[1,2,4]triazol-3-yl]-phenol (Example 270)

4-Hydroxy-2-[5-(5-trifluoromethyl-pyridin-2-yl)-2H-[1,2,4]triazol-3-yl]-phenol was prepared from N-amino-5-trifluoromethyl-pyridine-2-carboxamidine and 2,5-dihydroxybenzaldehyde using the protocol described in Example 36. [M+H]⁺ 323.2.

Compound 523: 2-[5-(3-Methoxy-phenyl)-1H-[1,2,4]triazol-3-yl]-5-trifluoromethyl-pyridine (Example 271)

2-[5-(3-Methoxy-phenyl)-1H-[1,2,4]triazol-3-yl]-5-trifluoromethyl-pyridine was prepared from N-amino-5-trifluoromethyl-pyridine-2-carboxamidine and 3-methoxy-benzaldehyde using the protocol described in Example 36. [M+H]⁺ 321.3.

Compound 524: 2-[5-(2-Fluoro-5-methoxy-phenyl)-1H-[1,2,4]triazol-3-yl]-5-trifluoromethyl-pyridine (Example 272)

2-[5-(2-Fluoro-5-methoxy-phenyl)-1H-[1,2,4]triazol-3-yl]-5-trifluoromethyl-pyridine was prepared from N-amino-5-trifluoromethyl-pyridine-2-carboxamidine and 2-fluoro-5-methoxy-benzaldehyde using the protocol described in Example 36. [M+H]⁺ 339.3.

Compound 525: 1-[5-(5-trifluoromethyl-pyridin-2-yl)-2H-[1,2,4]triazol-3-yl]-naphthalen-2-ol (Example 273)

1-[5-(5-trifluoromethyl-pyridin-2-yl)-2H-[1,2,4]triazol-3-yl]-naphthalen-2-ol was prepared from N-amino-5-trifluoromethyl-pyridine-2-carboxamidine and 2-hydroxy-naphthalene-1-carbaldehyde using the protocol described in Example 36. [M+H]⁺ 357.2.

Compound 526: 2-[5-(3-Bromo-phenyl)-1H-[1,2,4]triazol-3-yl]-5-trifluoromethyl-pyridine (Example 274)

2-[5-(3-Bromo-phenyl)-1H-[1,2,4]triazol-3-yl]-5-trifluoromethyl-pyridine was prepared from N-amino-5-trifluoromethyl-pyridine-2-carboxamidine and 3-bromo-benzaldehyde using the protocol described in Example 36. [M+H]⁺ 370.2.

Compound 527:Example 275: 2-[5-(2,6-Dichloro -phenyl)-1H-[1,2,4]triazol-3-yl]-5-trifluoromethyl-pyridine (Example 275)

2-[5-(2,6-Dichloro-phenyl)-1H-[1,2,4]triazol-3-yl]-5-trifluoromethyl-pyridine was prepared from N-amino-5-trifluoromethyl-pyridine-2-carboxamidine and 2,6-dichloro-benzaldehyde using the protocol described in Example 36. [M+H]⁺ 360.2.

Compound 528: 2-[5-(4-Bromo-2-fluoro-phenyl)-1H-[1,2,4]triazol-3-yl]-5-trifluoromethyl-pyridine (Example 276)

2-[5-(4-Bromo-2-fluoro-phenyl)-1H-[1,2,4]triazol-3-yl]-5-trifluoromethyl-pyridine was prepared from N-amino-5-trifluoromethyl-pyridine-2-carboxamidine and 4-bromo-2-fluoro-benzaldehyde using the protocol described in Example 36. [M+H]⁺ 388.3.

Compound 529: 2-[5-(5-Bromo-2-methoxy-phenyl)-1H-[1,2,4]triazol-3-yl]-5-trifluoromethyl-pyridine (Example 277)

2-[5-(5-Bromo-2-methoxy-phenyl)-1H-[ 1,2,4]triazol-3-yl]-5-trifluoromethyl-pyridine was prepared from N-amino-5-trifluoromethyl-pyridine-2-carboxamidine and 5-bromo-2-methoxy-benzaldehyde using the protocol described in Example 36. [M+H]⁺ 400.3.

Compound 556: 4-Bromo-2-[5-(3-nitro-pyridin-2-yl)-2H-[1,2,4]triazol-3-yl]-phenol (Example 278)

Compound 555: 2-[5-(3-amino-pyridin-2-yl)-2H-[1,2,4]triazol-3-yl]-4-bromo-phenol 556 & 555 (Example 279)

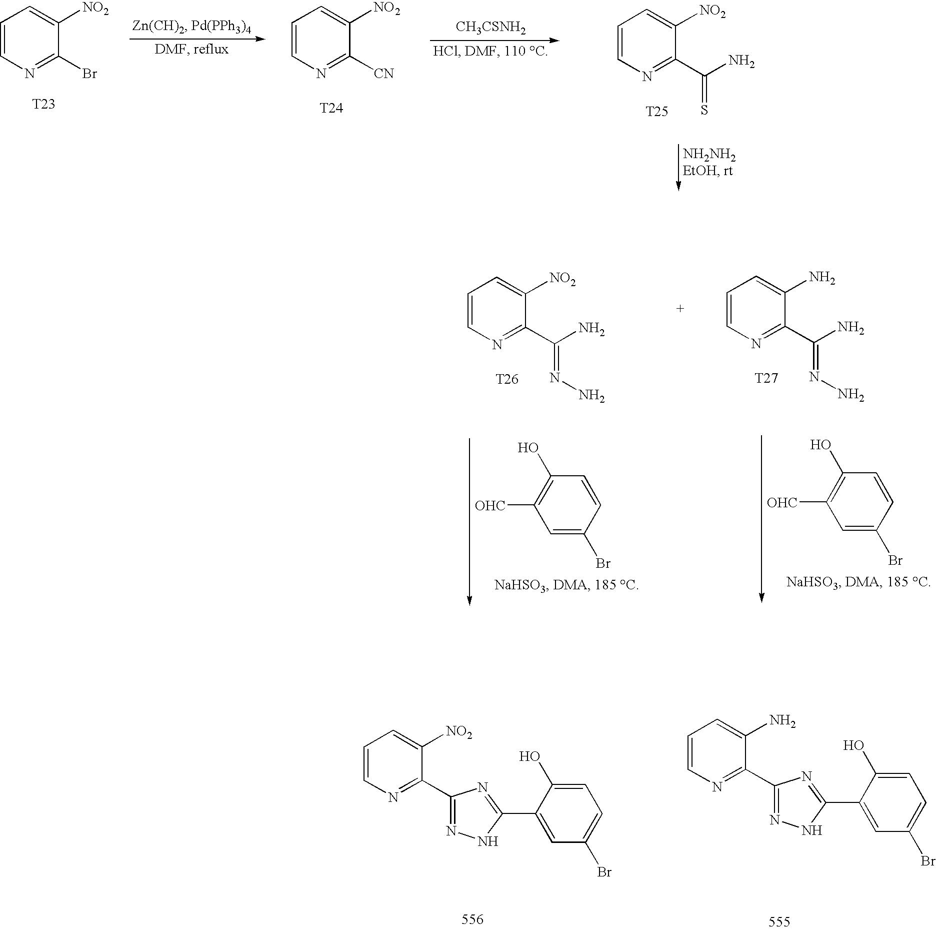

Step 1: Preparation of 3-nitro-pyridine-2-carbonitrile

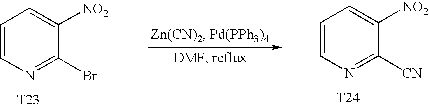

To a solution of 2-bromo-3-nitropyridine (6.1 g, 30 mmol) was added zinc cyanide (2.1 g, 18 mmol) and Pd(PPh₃)₄ (2.1 g, 1.8 mmol). The reaction mixture was purged with argon for 10 min and refluxed under argon for 3 h. The reaction mixture was re-partitioned between ethyl acetate and water. The aqueous layer was separated and extrated two times with ethyl acetate. The combined organic layers were washed with brine and dried over anhydrous MgSO4. After removal of the solvent, the residue was purified by column chromatography (30% EtOAc-Hexane) to give 3-Nitro-pyridine-2-carbonitrile (4.1 g, 92%). 1H NMR (400 MHz, CHLOROFORM-D) δ ppm 7.44-7.51 (m, 1 H) 8.13 (dd, J=8.07, 1.83 Hz, 1 H) 8.58 (dd, J=4.58, 1.65 Hz, 1 H).

Step 2: Preparation of 3-Nitro-3-pyridine-2-carbothioic acid amide

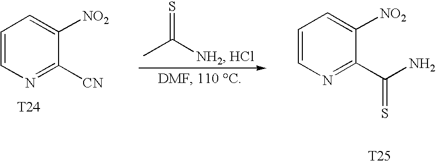

3-Nitro-pyridine-2-carbonitrile (1.5 g, 10 mmol) and thioacetamide ( 1.5 g, 20 mmol) was dissolved in DMF (20 mL). Hydrogen chloride was bubbled through the reaction solution for 10 min. The reaction mixture was heated at 100 C. for 1 h, cooled to room temperature and re-partitioned between ethyl acetate and water. The aqueous layer was separated and extracted with ethyl acetate two times. The combined organic layers were washed with brine and dried over anhydrous MgSO₄. After removal of the solvent, the residue was purified by column chromatography (40-50% EtOAc-Hexane) to give 3-nitro-pyridine-2-carbothioic acid amide (1.32 g, 72%). 1H NMR (400 MHz, DMSO-D6) δ ppm 7.65 (dd, J=8.43, 4.77 Hz, 1 H) 8.41 (d, J=8.07 Hz, 1 H) 8.75 (d, J=4.40 Hz, 1 H) 10.07 (s, 1 H) 10.38 (s, 1 H).

Step 3: Preparation of N-Amino-3-nitro-pyridine-2-carboxamidine and N-amino-3 amino-pyridine-2-carboxamidine

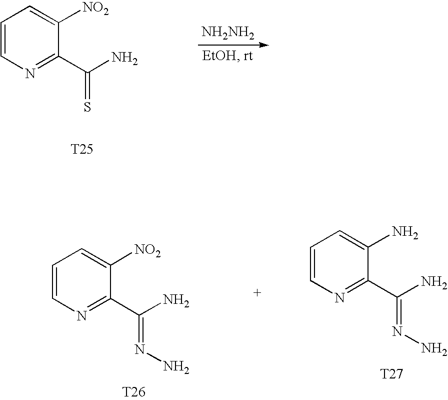

3-Nitro-pyridine-2-carboxamidine (183 mg, 1.0 mmol) and hydrazine hydrate (100 mg, 2.0 mmol) were dissolved in mthanol (5 mL). The reaction mixture was stirred at room temperature for 3 hr. After removal of the solvent, the residue was partitioned between water and ethyl acetate. The aqueous layer was separated and extracted with ethyl acetate. The combined organic layers were washed with brine, dried over anhydrous magnesium sulfate and evaporated to dryness to yield a mixture of N-amino-3-nitro-pyridine-2-carboxamidine and N-amino-3-amino-pyridine-2-carboxamidine that was used without further purification.

Step 4

Compound 556: 4-bromo-2-[5-(3-nitro-pyridin-2-yl)-2H-[1,2,4]triazol-3-yl]-phenol

Compound 555: 4-bromo-2-[5-(3-amino-pyridin-2-yl)-2H-[1,2,4]triazol-3-yl]-phenol

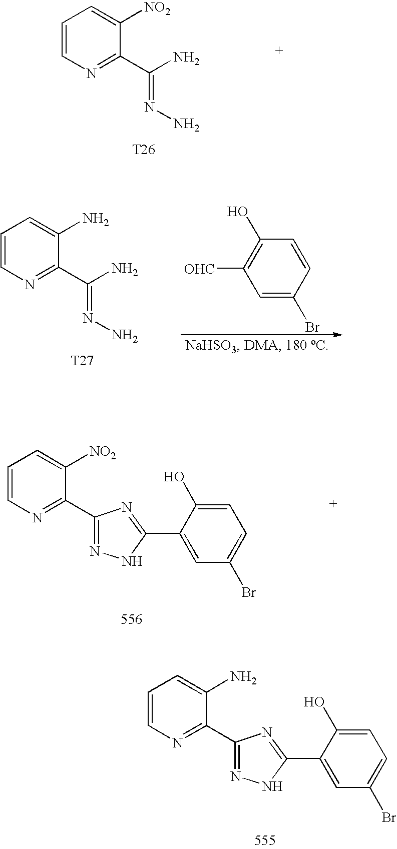

A mixture of N-amino-3-nitro-pyridine-2-carboxamidine and N-amino-3-amino-pyridine-2-carboxamidine, 5-Bromo-2-hydroxy-benzaldehyde (20 mg, 0.1 mmol) and sodium hydrogensulfite (15.6 mg, 0.15 mmol) was dissolved in N,N-dimethylacetamide (0.2 mL). The reaction mixture was heated at 185° C. for 2 h, cooled to room temperature and purified using LC/MS to give 4-bromo-2-[5-(3-nitro-pyridin-2-yl)-2H-[1,2,4]triazol-3-yl]-phenol ([M+H]⁺ 362.2) and 4-bromo-2-[5-(3-amino-pyridin-2-yl)-2H-[1,2,4]triazol-3-yl]-phenol ( [M+H]⁺ 332.2).

Compound 554: 4-Methoxy-2-[5-(3-nitro-pyridin-2-yl)-2H-[1,2,4]triazol-3-yl]-phenol (Example 280)

4-Methoxy-2-(5-(3-nitro-pyridin-2-yl)-2H-[1,2,4]triazol-3-yl)-phenol was prepared from N-amino-3-nitro-pyridine-2-carboxamidine and 5-methoxy-2-hydroxy-benzaldehyde using the protocol described in Example 36, step 4. [M+H]⁺ 314.3.

Compound 558: 1-[5-(3-Nitro-pyridin-2-yl)-2H-[1,2,4]triazol-3-yl]-naphthalen-2-ol (Example 281)

Compound 557: 1-[5-(3-amino-pyridin-2-yl)-2H-[1,2,4]triazol-3-yl]-naphthalen-2-ol (Example 282)

1-[5-(3-Nitro-pyridin-2-yl)-2H-[1,2,4]triazol-3-yl]-naphthalen-2-ol and 1-[5-(3-amino-pyridin-2-yl)-2H-[1,2,4]triazol-3-yl]-naphthalen-2-ol were prepared from the mixture of N-amino-3-nitro-pyridine-2-carboxamidine and N-amino-3-amino-pyridine-2-carboxamidine and 2-hydroxy-naphthalene-1-carbaldehyde using the protocol described in Example 36, step 4. 1-[5-(3-Nitro-pyridin-2-yl)-2H-[1,2,4]triazol-3-yl]-naphthalen-2-ol: [M+H]⁺ 334.2; 1-[5-(3-amino-pyridin-2-yl)-2H-[1,2,4]triazol-3-yl]-naphthalen-2-ol: [M+H]⁺ 304.2.

Compound 592: 2-[5-(5-Amino-pyridin-2-yl)-2H-[1,2,4]triazol-3-yl]-4-bromo-phenol (Example 283)

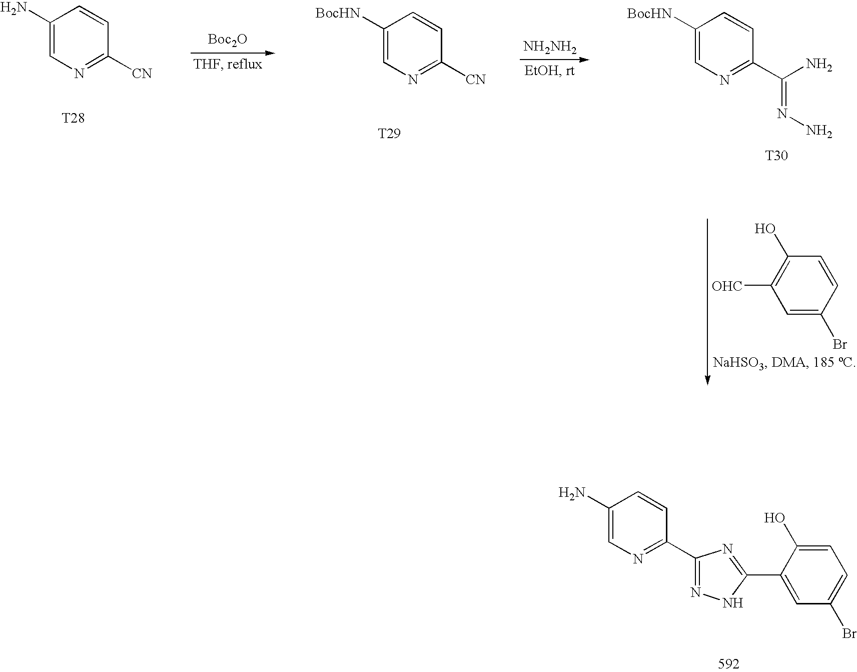

Step 1: Preparation of (6-cyano-pyridin-3-yl)-carbamic acid tert-butyl ester

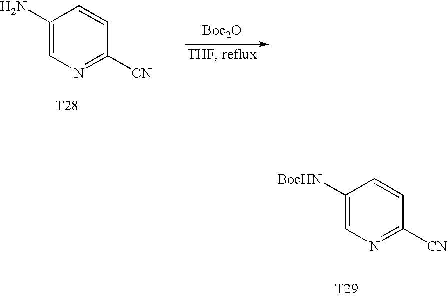

5-Amino-2-carbonitrile (4.8 g, 40 mmol) and di-t-butyl-dicarbonate (13.1 g, 60 mmol) was dissolved in THF (10 mL). The reaction mixture was refluxed overnight and concentrated. The residue was partitioned between ethyl acetate and water. The aqueous layer was separated and extracted two times with ethyl acetate. The combined organic layers were washed with brine and dried over anhydrous MgSO4. After removal of the solvent, the residue was purified by column chromatography (15% EtOAc-Hexane) to give (6-cyano-pyridin-3-yl)-carbamic acid tert-butyl ester ( 5.9 g, 67%). 1H NMR (400 MHz, CHLOROFORM-D) δ ppm 6.91 (s, 1 H) 7.62 (d, J=8.80 Hz, 1 H) 8.15 (d, J=8.43 Hz, 1 H) 8.49 (d, J=2.57 Hz, 1 H).

Step 2: Preparation of N-Amino-(6-Carbamimidoyl-pyridin-3-yl)-carbamic acid tert butyl ester

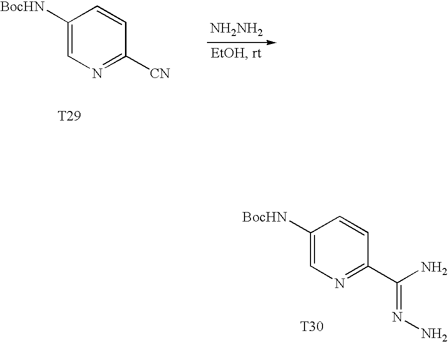

(6-cyano-pyridin-3-yl)-carbamic acid tert-butyl ester (219 mg, 1.0 mmol) and hydrazine hydrate (1.0 g, 20.0 mmol) were dissolved in CH₃CN (1 mL). The reaction mixture was stirred at room temperature for 48 hr. After removal of the solvent, the residue was partitioned between water and ethyl acetate. The aqueous layer was separated and extracted with ethyl acetate. The combined organic layers were washed with brine, dried over anhydrous magnesium sulfate and evaporated to dryness to yield N-amino-(6-carbamimidoyl-pyridin-3-yl)-carbamic acid tert-butyl ester that was used without further purification. [M+H]⁺ 252.2; 1H NMR (400 MHz, DMSO-D6) δ ppm 5.14 (s, 2 H) 5.61 (s, 2 H) 7.74-7.85 (m, 2 H) 8.48-8.54 (m, 1 H) 9.57 (s, 1 H).

Step 3

Compound 592: 2-15-(5-Amino-pyridin-2-yl)-2H-[1,2,4]triazol-3-yl]-4-bromo-phenol

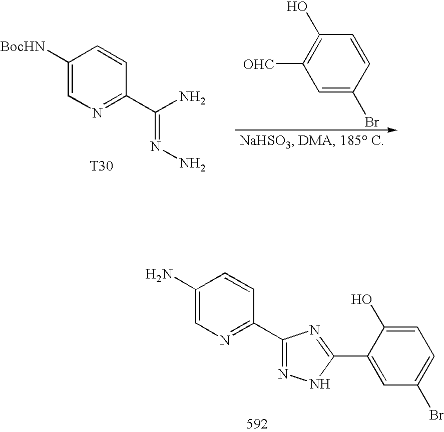

2-[5-(5-Amino-pyridin-2-yl)-2H-[1,2,4]triazol-3-yl]-4-bromo-phenol was prepared from N-amino-(6-carbamimidoyl-pyridin-3-yl)-carbamic acid tert-butyl ester and 5-bromo-2-hydroxy-benzaldehyde using the protocol described in Example 36. [M+H]⁺ 332.2; 1H NMR (400 MHz, DMSO-D6) δ ppm 5.99 (s, 2 H) 6.95 (d, J=8.80 Hz, 1 H) 7.05 (d, J=8.80 Hz, 1 H) 7.44 (d, J=8.80 Hz, 1 H) 7.85 (d, J=8.80 Hz, 1 H) 8.04 (s, 2 H) 11.21 (s, 1 H).

Compound 593: N-{6-[5-(5-Bromo-2-hydroxy-phenyl)-1H-[1,2,4]triazol-3-yl]-pyridin-3-yl}-acetamide (Example 284)

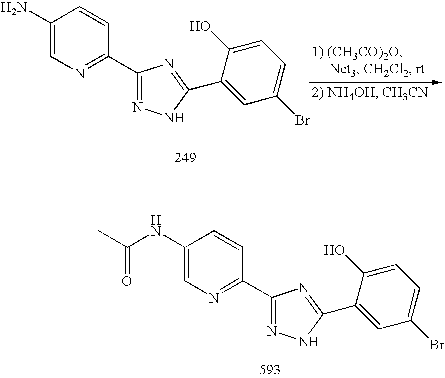

To a solution of 2-[5-(5-Amino-pyridin-2-yl)-2H-[1,2,4]triazol-3-yl]-4-bromo-phenol (33 mg, 0.1 mmol) in CH₂Cl₂ (1 mL) was added triethylamine (55 μL, 0.4 mmol) and acetic anhydride (40 μL, 0.4 mmol). The reaction mixture was stirred at room temperature overnight. After removal of the solvent, the residue was re-taken into CH₃CN (1 mL) and ammonium hydroxide ( 0.2 mL) was added. The reaction mixture was stirred at room temperature for 2 h and purified using preparative LC/MS. [M+H]⁺ 374.2; 1H NMR (400 MHz, DMSO-D6) δ ppm 2.12 (s, 3 H) 6.98 (d, J=8.80 Hz, 1 H) 7.46 (dd, J=9.16, 2.20 Hz, 1 H) 8.07-8.26 (m, 3 H) 8.86 (s, 1 H) 10.39 (s, 1 H).

Compound 594: 2-[5-(5-Amino-pyridin-2-yl)-2H-[1,2,4]triazol-3-yl]-4-methoxy-phenol (Example 285)

2-[5-(5-Amino-pyridin-2-yl)-2H-[1,2,4]triazol-3-yl]-4-methoxy-phenol was prepared from N-amino-(6-carbamimidoyl-pyridin-3-yl)-carbamic acid tert-butyl ester and 5-methoxy-2-hydroxy-benzaldehyde using the protocol described in Example 36. [M+H]⁺ 284.2; 1H NMR (400 MHz, DMSO-D6) δ ppm 3.75 (s, 3 H) 5.96 (s, 2 H) 6.89 (s, 2 H) 7.48 (s, 1 H) 7.83 (d, J=8.43 Hz, 1 H) 8.04 (s, 1 H) 10.74 (s, 1 H).

Compound 595: N-{6-[5-(5-methoxy-2-hydroxy-phenyl)-1H-[1,2,4]triazol-3-yl]-pyridin-3-yl}-acetamide (Example 286)

N-{6-[5-(5-methoxy-2-hydroxy-phenyl)-1H-[1,2,4]triazol-3-yl]-pyridin-3-yl}-acetamide was prepared from 2-[5-(5-Amino-pyridin-2-yl)-2H-[1,2,4]triazol-3-yl]-4-methoxy-phenol using the protocol described in Example 36. [M+H]⁺ 326.2.

Compound 596: 4-Bromo-2-(2-methyl-5-pyridin-2-yl-2H-[1,2,4]triazol-3-yl)-phenol (Example 287)

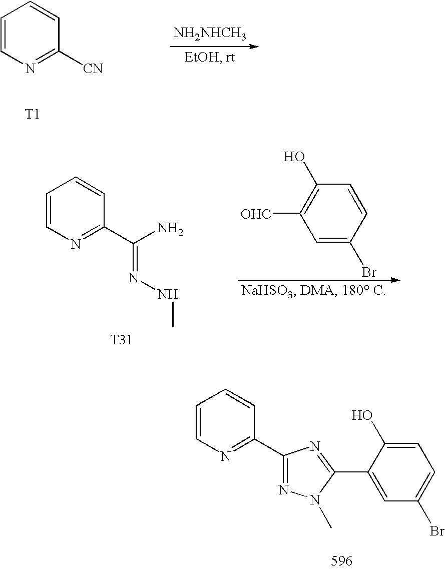

Step 1: Preparation of N-Methylamino-piridine-2-carboxamidine

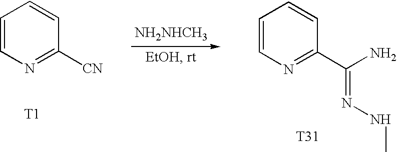

Pyridine-2-carbonitrile (2.1 g, 20.0 mmol) and methyl-hydrazine (4.6 g, 100.0 mmol) were dissolved in ethanol (10 mL). The reaction mixture was stirred at room temperature for 72 hr. After removal of the solvent, the residue was partitioned between water and ethyl acetate. The aqueous layer was separated and extracted with ethyl acetate. The combined organic layers 10 were washed with brine, dried over anhydrous magnesium sulfate and evaporated to dryness to yield N-methylamino-pyridine-2-carboxamidine (2.7 g, 90%) that was used without further purification. [M+H]⁺ 151.2; 1H NMR (400 MHz, DMSO-D6) δ ppm 2.79 (s, 3 H) 4.97 (s, 1 H) 5.69 (s, 2 H) 7.20-7.36 (m, 1 H) 7.61-7.75 (m, 1 H) 7.85-7.98 (m, 1 H) 8.38-8.53 (m, 1 H).

Step 2

Compound 596: Preparation 4-Bromo-2-(2-methyl-5-pyridin-2-yl-2H-[1,2,4]triazol-3-yl)-phenol

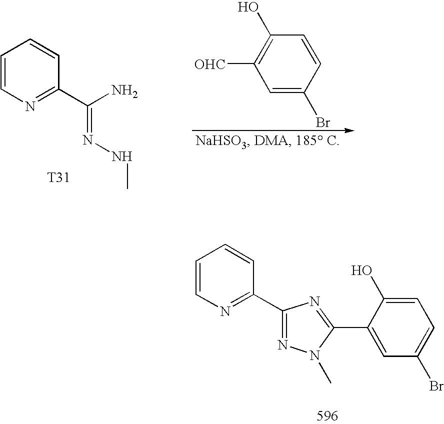

4-Bromo-2-(2-methyl-5-pyridin-2-yl-2H-[1,2,4]triazol-3-yl)-phenol was prepared from N-methylamino-pyridine-2-carboxamidine and 5-bromo-2-hydroxy-benzaldehyde using the protocol described in Example 36. [M+H]⁺ 331.2. 1H NMR (400 MHz, DMSO-D6) δ ppm 3.82 (s, 3 H) 6.99 (d, J=8.80 Hz, 1 H) 7.38-7.43 (m, 1 H) 7.53-7.59 (m, 2 H) 7.85-7.91 (m, 1 H) 8.01-8.05 (m, 1 H) 10.71 (s, 1 H).

Compound 597: 4-Nitro-2-(2-methyl-5-pyridin-2-yl-2H-[1,2,4]triazol-3-yl)-phenol (Example 288)

4-Nitro-2-(2-methyl-5-pyridin-2-yl-2H-[1,2,4]triazol-3-yl)-phenol was prepared from N-methylamino-pyridine-2-carboxamidine and 5-nitro-2-hydroxy-benzaldehyde using the protocol described in Example 36. [M+H]⁺ 298.1.

Compound 598: 4-Methoxy-2-(2-methyl-5-pyridin-2-yl-2H-[1,2,4]triazol-3-yl)-phenol (Example 289)

4-Methoxy-2-(2-methyl-5-pyridin-2-yl-2H-[1,2,4]triazol-3-yl)-phenol was prepared from N-methylamino-pyridine-2-carboxamidine and 5-methoxy-2-hydroxy-benzaldehyde using the protocol described in Example 36. [M+H]⁺ 283.3.

Compound 599: 4-Chloro-2-(2-methyl-5-pyridin-2-yl-2H-[1,2,4]triazol-3-yl)-phenol (Example 290)

4-Chloro-2-(2-methyl-5-pyridin-2-yl-2H-[1,2,4]triazol-3-yl)-phenol was prepared from N-methylamino-pyridine-2-carboxamidine and 5-chloro-2-hydroxy-benzaldehyde using the protocol described in Example 36. [M+H]⁺ 287.3; 1H NMR (400 MHz, DMSO-D6) δ ppm 3.83 (s, 3 H) 7.04 (d, J=8.43 Hz, 1 H) 7.38-7.48 (m, 3 H) 7.84-7.92 (m, 1 H) 8.01-8.06 (m, 1 H) 10.68-10.71 (m, 1 H).

Compound 600: 5-Benzyloxy-2-(2-methyl-5-pyridin-2-yl-2H-[1,2,4]triazol-3-yl)-phenol (Example 291)

5-Benzyloxy-2-(2-methyl-5-pyridin-2-yl-2H-[1,2,4]triazol-3-yl)-phenol was prepared from N-methylamino-pyridine-2-carboxamidine and 4-benzyloxy-2-hydroxy-benzaldehyde using the protocol described in Example 36. [M+H]⁺ 359.2.

Compound 601: 5-Methoxy-2-(2-methyl-5-pyridin-2-yl-2H-[1,2,4]triazol-3-yl)-phenol (Example 292)

5-Methoxy-2-(2-methyl-5-pyridin-2-yl-2H-[1,2,4]triazol-3-yl)-phenol was prepared from N-methylamino-pyridine-2-carboxamidine and 4-methoxy-2-hydroxy-benzaldehyde using the protocol described in Example 36. [M+H]⁺ 283.3.

Compound 602: 2-(2-methyl-5-pyridin-2-yl-2H-[1,2,4]triazol-3-yl)-phenol (Example 293)

2-(2-methyl-5-pyridin-2-yl-2H-[1,2,4]triazol-3-yl)-phenol was prepared from N-methylamino-pyridine-2-carboxamidine and 2-hydroxy-benzaldehyde using the protocol described in Example 36. [M+H]⁺ 253.2.

Compound 603: 6-Methoxy-2-(2-methyl-5-pyridin-2-yl-2H-[1,2,4]triazol-3-yl)-phenol (Example 294)

6-Methoxy-2-(2-methyl-5-pyridin-2-yl-2H-[1,2,4]triazol-3-yl)-phenol was prepared from N-methylamino-pyridine-2-carboxamidine and 3-methoxy-2-hydroxy-benzaldehyde using the protocol described in Example 36. [M+H]⁺ 283.3.

Compound 604: 4-Hydroxy-2-(2-methyl-5-pyridin-2-yl-2H-[1,2,4]triazol-3-yl)-phenol (Example 295)

4-Hydroxy-2-(2-methyl-5-pyridin-2-yl-2H-[1,2,4]triazol-3-yl)-phenol was prepared from N-methylamino-pyridine-2-carboxamidine and 2,5-dihydroxybenzaldehyde using the protocol described in Example 36. [M+H]⁺ 269.2.

Compound 605: 1-(2-methyl-5-Pyridin-2-yl-2H-[1,2,4]triazol-3-yl)-naphthalen-2-ol (Example 296)

1-(2-methyl-5-Pyridin-2-yl-2H-[ 1,2,4]triazol-3-yl)-naphthalen-2-ol was prepared from N-methylamino-pyridine-2-carboxamidine and 2-hydroxy-naphthalene-1-carbaldehyde using the protocol described in Example 36. [M+H]⁺ 303.2.

Compound 606: 2-(2-Methyl-5-pyridin-2-yl-2H-[1,2,4]triazol-3-yl)-naphthalen-1-ol (Example 297)

2-(2-Methyl-5-pyridin-2-yl-2H-[ 1,2,4]triazol-3-yl)-naphthalen-1-ol was prepared from N-methylamino-pyridine-2-carboxamidine and 1-hydroxy-naphthalene-2-carbaldehyde using the protocol described in Example 36. [M+H]⁺ 303.2.

Compound 625: 4,6-Dichloro-2-(2-methyl-5-pyridin-2-yl-2H-[1,2,4]triazol-3-yl)-phenol (Example 298)

4,6-Dichloro-2-(2-methyl-5-pyridin-2-yl-2H-[1,2,4]triazol-3-yl)-phenol was prepared from N-methylamino-pyridine-2-carboxamidine and 4,6-dichloro-2-hydroxy-benzaldehyde using the protocol described in Example 36. [M+H]⁺ 322.1. 1H NMR (400 MHz, DMSO-D6) δ ppm 3.91 (s, 3 H) 7.38-7.52 (m, 1 H) 7.58 (d, J=2.34 Hz, 1 H) 7.77 (d, J=2.73 Hz, 1 H) 7.87-8.01 (m, 1 H) 8.02-8.15 (m, 1 H) 8.58-8.74 (m, 1 H) 11.01 (s, 1 H).

Compound 626: 4,6-Dibromo -2-(2-methyl-5-pyridin-2-yl-2H-[1,2,4]triazol-3-yl)-phenol (Example 299)

4,6-Dibromo-2-(2-methyl-5-pyridin-2-yl-2H-[1,2,4]triazol-3-yl)-phenol was prepared from N-methylamino-pyridine-2-carboxamidine and 4,6-dibromo-2-hydroxy-benzaldehyde using the protocol described in Example 36. [M+H]⁺ 412.2. 1H NMR (400 MHz, DMSO-D6) δ ppm 3.93 (s, 3 H) 7.44-7.50 (m, 1 H) 7.74 (d, J=2.34 Hz, 1 H) 7.91-7.96 (m, 1 H) 7.98 (d, J=2.34 Hz, 1 H) 8.06-8.11 (m, 1 H) 8.64-8.70 (m, 1 H) 11.06 (bs, 1 H).

Compound 627: 6-Bromo-4-Chloro-2-(2-methyl-5-pyridin-2-yl-2H-[1,2,4]triazol-3-yl)-phenol (Example 300)

6-Bromo-4-chloro-2-(2-methyl-5-pyridin-2-yl-2H-[1,2,4]triazol-3-yl)-phenol was prepared from N-methylamino-pyridine-2-carboxamidine and 4-chloro6-bromo-2-hydroxy-benzaldehyde using the protocol described in Example 36. [M+H]⁺ 368.2; 1H NMR (400 MHz, DMSO-D6) δ ppm 3.94 (s, 3 H) 7.45-7.51 (m, 1 H) 7.64 (d, J=2.34 Hz, 1 H) 7.89 (d, J=2.73 Hz, 1 H) 7.92-7.99 (m, 1 H) 8.06-8.13 (m, 1 H) 8.64-8.70 (m, 1 H) 11.08 (s, 1 H).

Compound 612: 4-Bromo-2-[2-methyl-5-(3-methyl-pyridin-2-yl)-2H-[1,2,4]triazol-3-yl]phenol (Example 301)

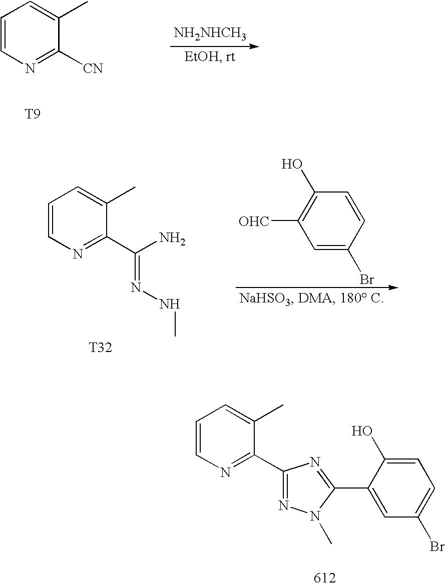

Step 1: Preparation of N-Methylamino-3-Methyl-pyridine-2-carboxamidine

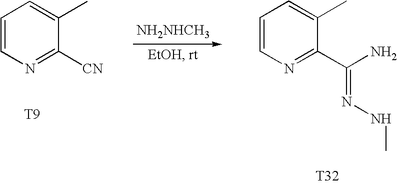

3-Methyl-pyridine-2-carbonitrile (1.18 g, 10.0 mmol) and methyl-hydrazine (9.2 g, 200.0 mmol) were dissolved in ethanol (5 mL). The reaction mixture was heated at 90° C. for 24 hr. After removal of the solvent, the residue was partitioned between water and ethyl acetate. The aqueous layer was separated and extracted with ethyl acetate. The combined organic layers were washed with brine, dried over anhydrous magnesium sulfate and evaporated to dryness to yield N-methylamino-3-methyl-pyridine-2-carboxamidine (890 mg, 54%) that was used without further purification. [M+H]⁺ 165.2.

Step 2

Compound 612: 4-Bromo-2-[2-methyl-5-(3-methyl-pyridin-2-yl)-2H-[1,2,4]triazol-3-yl]-phenol

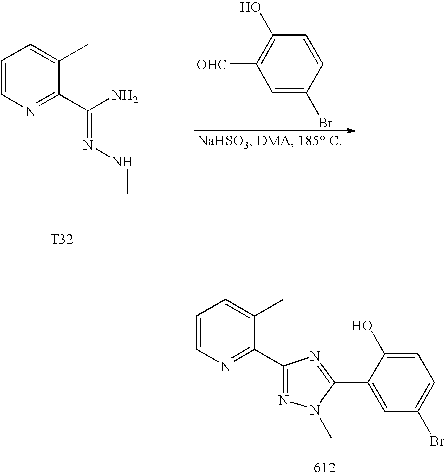

4-Bromo-2-[2-methyl-5-(3-methyl-pyridin-2-yl)-2H-[1,2,4]triazol-3-yl]-phenol was prepared from N-methylamino-3-methyl-pyridine-2-carboxamidine and 5-bromo-2-hydroxy-benzaldehyde using the protocol described in Example 36. [M+H]⁺ 346.2.

Compound 607: 4-Nitro-2-[2-methyl-5-(3-methyl-pyridin-2-yl)-2H-[1,2,4]triazol-3-yl]-phenol (Example 302)

4-Nitro-2-[2-methyl-5-(3-methyl-pyridin-2-yl)-2H-[1,2,4]triazol-3-yl]-phenol was prepared from N-methylamino-3-methyl-pyridine-2-carboxamidine and 5-nitro-2-hydroxy-benzaldehyde using the protocol described in Example 36. [M+H]⁺ 312.1. 1H NMR (400 MHz, DMSO-D6) δ ppm 2.55 (s, 3 H) 3.87 (s, 3 H) 7.15-7.28 (m, 1 H) 7.35 (dd, J=7.80, 4.68 Hz, 1 H) 7.74 (dd, J=7.80, 1.17 Hz, 1 H) 8.25-8.38 (m, 2 H) 8.48 (dd, J=4.68, 1.17 Hz, 1 H) 12.07 (s, 1 H).

Compound 608: 4-Fluoro-2-[2-methyl-5-(3-methyl-pyridin-2-yl)-2H-[1,2,4]triazol-3-yl]-phenol (Example 303)

4-Fluoro-2-[2-methyl-5-(3-methyl-pyridin-2-yl)-2H-[1,2,4]triazol-3-yl]-phenol was prepared from N-methylamino-3-methyl-pyridine-2-carboxamidine and 5-fluoro-2-hydroxy-benzaldehyde using the protocol described in Example 36. [M+H]⁺ 285.1; 1H NMR (400 MHz, DMSO-D6) δ ppm 2.54 (s, 3 H) 3.87 (s, 3 H) 7.03 (dd, J=8.97, 4.68 Hz, 1 H) 7.23-7.27 (m, 1 H) 7.27-7.32 (m, 1 H) 7.34 (dd, J=7.80, 4.68 Hz, 1 H) 7.73 (dd, J=6.83, 1.76 Hz, 1 H) 8.48 (dd, J=4.68, 1.17 Hz, 1 H) 10.48 (s, 1 H).

Compound 609: 4-Methoxy-2-[2-methyl-5-(3-methyl-pyridin-2-yl)-2H-[1,2,4]triazol-3-yl]-phenol (Example 304)

4-Methoxy-2-[2-methyl-5-(3-methyl-pyridin-2-yl)-2H-[1,2,4]triazol-3-yl]-phenol was prepared from N-methylamino-3-methyl-pyridine-2-carboxamidine and 5-methoxy-2-hydroxy-benzaldehyde using the protocol described in Example 36. [M+H]⁺ 297.3; 1H NMR (400 MHz, DMSO-D6) δ ppm 2.55 (s, 3 H) 3.73 (s, 3 H) 3.87 (s, 3 H) 6.94-7.04 (m, 3 H) 7.33-7.39 (m, 1 H) 7.75 (d, J=7.80 Hz, 1 H) 8.48 (d, J=4.29 Hz, 1 H) 9.98 (s, 1 H).

Compound 610: 4-Chloro-2-[2-methyl-5-(3-methyl-pyridin-2-yl)-2H-[1,2,4]triazol-3-yl]-phenol (Example 305)

4-Chloro-2-[2-methyl-5-(3-methyl-pyridin-2-yl)-2H-[1,2,4]triazol-3-yl]-phenol was prepared from N-methylamino-3-methyl-pyridine-2-carboxamidine and 5-chloro-2-hydroxy-benzaldehyde using the protocol described in Example 36. [M+H]⁺ 301.3; 1H NMR (400 MHz, DMSO-D6) δ ppm 2.54 (s, 3 H) 3.85 (s, 3 H) 7.05 (d, J=8.97 Hz, 1 H) 7.34 (dd, J=7.41, 4.68 Hz, 1 H) 7.42-7.49 (m, 2 H) 7.73 (dd, J=7.80, 1.56 Hz, 1 H) 8.47 (dd, J=5.07, 1.95 Hz, 1 H) 10.74 (s, 1 H).

Compound 611: 5-Benzyloxy-2-[2-methyl-5-(3-methyl-pyridin-2-yl)-2H-[1,2,4]triazol-3-yl]-phenol (Example 306)

5-Benzyloxy-2-[2-methyl-5-(3-methyl-pyridin-2-yl)-2H-[1,2,4]triazol-3-yl]-phenol was prepared from N-methylamino-3-methyl-pyridine-2-carboxamidine and 4-benzyloxy-2-hydroxy-benzaldehyde using the protocol described in Example 36. [M+H]⁺ 373.2; 1H NMR (400 MHz, DMSO-D6) δ ppm 2.57 (s, 3 H) 3.90 (s, 3 H) 5.15 (d, J=10.14 Hz, 2 H) 6.61-6.68 (m, 3 H) 7.32-7.48 (m, 6 H) 7.80 (dd, J=7.41, 0.78 Hz, 1 H) 8.48-8.52 (m, 1 H) 10.98 (s, 1 H).

Compound 613: 5-Methoxy-2-[2-methyl-5-(3-methyl-pyridin-2-yl)-2H-[1,2,4]triazol-3-yl]-phenol (Example 307)

5-Methoxy-2-[2-methyl-5-(3-methyl-pyridin-2-yl)-2H-[1,2,4]triazol-3-yl]-phenol was prepared from N-methylamino-3-methyl-pyridine-2-carboxamidine and 4-methoxy-2-hydroxy-benzaldehyde using the protocol described in Example 36. [M+H]⁺ 297.3; 1H NMR (400 MHz, DMSO-D6) δ ppm 2.49 (s, 3 H) 4.07 (s, 3 H) 6.97 (d, J=8.80 Hz, 1 H) 7.47 (dd, J=8.80, 2.57 Hz, 1 H) 7.52 (dd, J=7.70, 4.77 Hz, 1 H) 7.81-7.96 (m, 1 H) 8.03 (d, J=2.57 Hz, 1 H) 8.54-8.66 (m, 1 H) 10.83 (s, 1 H).

Compound 614: 2-[2-methyl-5-(3-methyl-pyridin-2-yl)-2H-[1,2,4]triazol-3-yl]-phenol (Example 308)

2-[2-methyl-5-(3-methyl-pyridin-2-yl)-2H-[1,2,4]triazol-3-yl]-phenol was prepared from N-methylamino-3-methyl-pyridine-2-carboxamidine and 2-hydroxy-benzaldehyde using the protocol described in Example 36. [M+H]⁺ 267.2; 1H NMR (400 MHz, DMSO-D6) δ ppm 6.96 (t, J=7.70 Hz, 1 H) 7.03 (d, J=8.07 Hz, 1 H) 7.31-7.42 (m, 2 H) 7.44-7.50 (m, 1 H) 7.72 (d, J=7.70 Hz, 1 H) 8.47 (d, J=6.23 Hz, 1 H) 10.52 (s, 1 H).

Compound 615: 6-Methoxy-2-[2-methyl-5-(3-methyl-pyridin-2-yl)-2H-[1,2,4]triazol-3-yl]-phenol (Example 309)

6-Methoxy-2-[2-methyl-5-(3-methyl-pyridin-2-yl)-2H-[1,2,4]triazol-3-yl]-phenol was prepared from N-methylamino-3-methyl-pyridine-2-carboxamidine and 3-methoxy-2-hydroxy-benzaldehyde using the protocol described in Example 36. [M+H]⁺ 297.3; 1H NMR (400 MHz, DMSO-D6) δ ppm 2.54 (s, 3 H) 3.85 (s, 3 H) 3.87 (s, 3 H) 6.93 (t, J=8.00 Hz, 1 H) 7.04-7.08 (m, 1 H) 7.15 (dd, J=8.19, 1.56 Hz, 1 H) 7.34 (dd, J=7.80, 4.68 Hz, 1 H) 7.73 (dd, J=7.22, 1.36 Hz, 1 H) 8.47 (dd, J=4.68, 1.56 Hz, 1 H) 9.85 (s, 1 H).

Compound 616: 4-Hydroxy-2-[2-methyl-5-(3-methyl-pyridin-2-yl)-2H-[1,2,4]triazol-3-yl]-phenol (Example 310)

4-Hydroxy-2-[2-methyl-5-(3-methyl-pyridin-2-yl)-2H-[1,2,4]triazol-3-yl]-phenol was prepared from N-methylamino-3-methyl-pyridine-2-carboxamidine and 2,5-dihydroxybenzaldehyde using the protocol described in Example 36. [M+H]⁺ 283.2. 1H NMR (400 MHz, DMSO-D6) δ ppm 2.54 (s, 3 H) 3.86 (s, 3 H) 6.78-6.83 (m, 1 H) 6.84 (s, 1 H) 6.85-6.87 (m, 1 H) 7.34 (dd, J=7.80, 4.68 Hz, 1 H) 7.71-7.75 (m, 1 H) 8.46-8.49 (m, 1 H) 9.05 (s, 1 H) 9.78 (s, 1 H).

Compound 617: 1-[2-methyl-5-(3-methyl-pyridin-2-yl)-2H-[1,2,4]triazol-3-yl]-naphthalen-2-ol (Example 311)

1-[2-methyl-5-(3-methyl-pyridin-2-yl)-2H-[1,2,4]triazol-3-yl]-naphthalen-2-ol was prepared from N-methylamino-3-methyl-pyridine-2-carboxamidine and 2-hydroxy-naphthalene-1-carbaldehyde using the protocol described in Example 36. [M+H]⁺ 317.2.

Compound 618: 2-[2-methyl-5-(3-methyl-pyridin-2-yl)-2H-[1,2,4]triazol-3-yl]-naphthalen-1-ol (Example 312)

2-[2-methyl-5-(3-methyl-pyridin-2-yl)-2H-[1,2,4]triazol-3-yl]-naphthalen-1-ol was prepared from N-methylamino-3-methyl-pyridine-2-carboxamidine and 1-hydroxy-naphthalene-2-carbaldehyde using the protocol described in Example 36. [M+H]⁺ 317.2; 1H NMR (400 MHz, DMSO-D6) δ ppm 2.63 (s, 3 H) 4.19 (s, 3 H) 7.41 (dd, J=7.80, 4.68 Hz, 1 H) 7.54-7.66 (m, 3 H) 7.81 (dd, J=8.58, 0.78 Hz, 1 H) 7.84 (d, J=8.58 Hz, 1 H) 7.92-7.96 (m, 1 H) 8.34 (dd, J=7.80, 1.17 Hz, 1 H) 8.55 (dd, J=4.88, 2.15 Hz, 1 H) 12.35 (s, 1 H).

Compound 633: 4-Bromo-2-[2-methyl-5-(3-trifluoromethyl-pyridin-2-yl)-2H-[1,2,4]triazol-3-yl]phenol (Example 313)

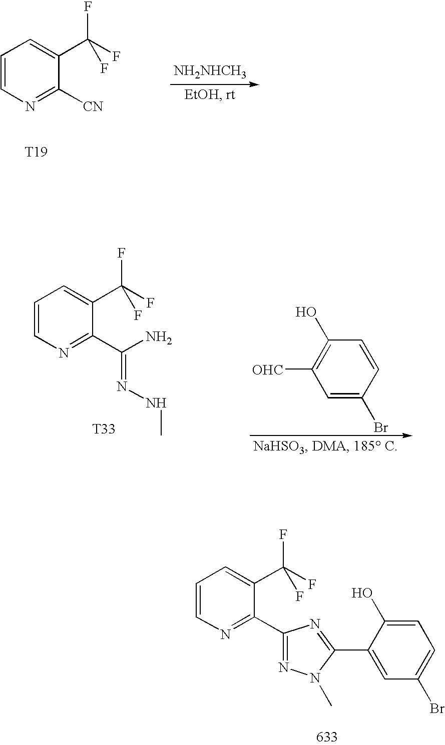

Step 1: Preparation of N-Methylamino-3-Trifluoromethyl-pyridine-2-carboxamidine

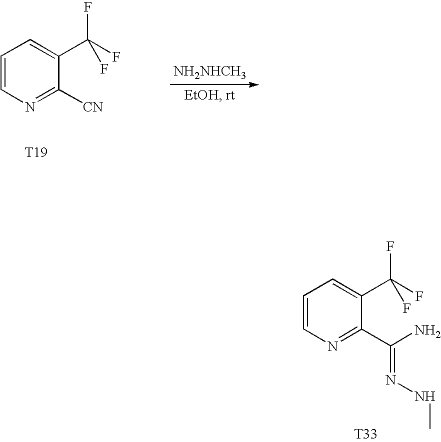

3-Trifluoromethyl-pyridine-2-carbonitrile (344 mg, 2.0 mmol) and methyl-hydrazine (165 gl, 4.0 mmol) were dissolved in ethanol (1 mL). The reaction mixture was heated at 90° C. for 24 hr. After removal of the solvent, the residue was partitioned between water and ethyl acetate. The aqueous layer was separated and extracted with ethyl acetate. The combined organic layers were washed with brine, dried over anhydrous magnesium sulfate and evaporated to dryness to yield N-methylamino-3-methyl-pyridine-2-carboxamidine (380 mg, 87%) that was used without further purification. [M+H]⁺ 219.2

Step 2:

Compound 633: 4-Bromo-2-[2-methyl-5-(3-trifluoromethyl-pyridin-2-yl)-2H-[1,2,4]triazol-3-yl]-phenol

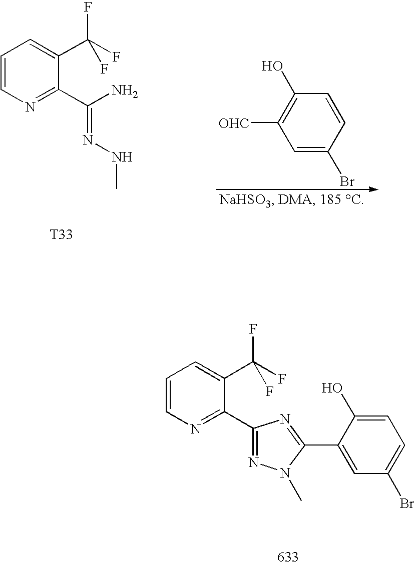

4-Bromo-2-[2-methyl-5-(3-trifluoromethyl-pyridin-2-yl)-2H-[1,2,4]triazol-3-yl]-phenol was prepared from N-methylamino-3-trifluoromethyl-pyridine-2-carboxamidine and 5-bromo-2-hydroxy-benzaldehyde using the protocol described in Example 36. [M+H]⁺ 399.2; 1H NMR (400 MHz, DMSO-D6) δ ppm 3.85 (s, 3 H) 7.01 (d, J=8.58 Hz, 1 H) 7.51 (d, J=2.73 Hz, 1 H) 7.56 (dd, J=8.78, 2.54 Hz, 1 H) 7.73 (dd, J=8.39, 4.49 Hz, 1 H) 8.34 (dd, J=8.19, 1.17 Hz, 1 H) 8.92 (dd, J=5.07, 1.17 Hz, 1 H) 10.75 (s, 1 H).

Compound 640: 4-Nitro-2-[2-methyl-5-(3-trifluoromethyl-pyridin-2-yl)-2H-[1,2,4]triazol-3-yl]-phenol (Example 314)

4-Nitro-2-[2-methyl-5-(3-trifluoromethyl-pyridin-2-yl)-2H-[1,2,4]triazol-3-yl]-phenol was prepared from N-methylamino-3-trifluoromethyl-pyridine-2-carboxamidine and 5-nitro-2-hydroxy-benzaldehyde using the protocol described in Example 36. [M+H]⁺ 312.1; 1H NMR (400 MHz, DMSO-D6) δ ppm 3.87 (s, 3 H) 7.23 (d, J=8.97 Hz, 1 H) 7.71-7.78 (m, 1 H) 8.26 (d, J=2.73 Hz, 1 H) 8.29-8.38 (m, 2 H) 8.94 (dd, J=4.68, 1.17 Hz, 1 H) 12.09 (bs, 1 H).

Compound 632: 4-Methoxy-2-[2-methyl-5-(3-trifluoromethyl-pyridin-2-yl)-2H-[1,2,4]triazol-3-yl]-phenol (Example 315)

4-Methoxy-2-[2-methyl-5-(3-trifluoromethyl-pyridin-2-yl)-2H-[1,2,4]triazol-3-yl]-phenol was prepared from N-methylamino-3-trifluoromethyl-pyridine-2-carboxamidine and 5-methoxy-2-hydroxy-benzaldehyde using the protocol described in Example 36. [M+H]⁺ 351.3. 1H NMR (400 MHz, DMSO-D6) δ ppm 3.72 (s, 3 H) 3.87 (s, 3 H) 6.92-7.04 (m, 3 H) 7.70-7.77 (m, 1 H) 8.33 (dd, J=8.19, 1.56 Hz, 1 H) 8.93 (dd, J=4.68, 1.17 Hz, 1 H) 9.94 (s, 1 H).

Compound 628: 2-(3-Phenyl-[1,2,4]triazol-1-yl)-pyridine (Example 316)

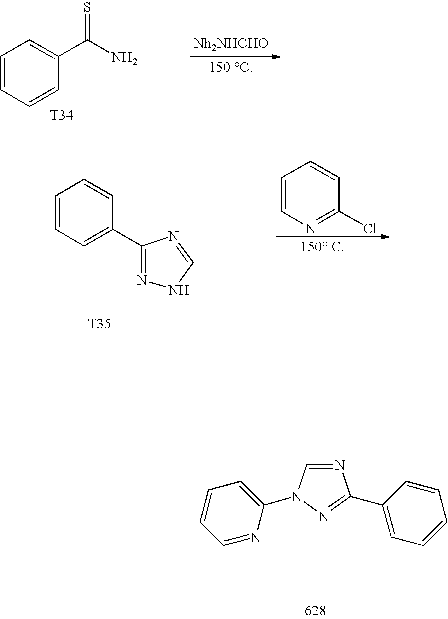

Step 1: Preparation of 3-Phenyl-1H-[1,2,4]triazole

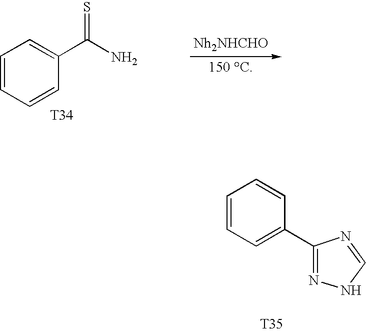

A mixture of thiobenzamide (2.74 g, 20 mmol) and formic acid hydrazide ( 2.4 g, 40 mmol) was purged with argon for 5 min and heated in a sealed tube at 150° C. for 4 hr. The reaction crude was used without further purification. [M+H]⁺ 147.1.

Step 2

Compound 628: 2-(3-Phenyl-[1,2,4]triazol-1-yl)-pyridine

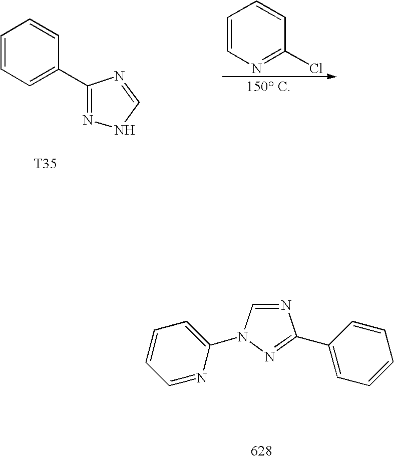

A mixture of 3-phenyl-1H-[1,2,4]triazole (219 mg, 1.5 mmol) and 2-chloropyridine (204 mg, 1.8 mmol) was purged with argon for 5 min and heated in a sealed tube at 130° C. for 24 hr. The reaction mixture was partitioned between water and ethyl acetate. The aqueous layer was separated and extracted three times with ethyl acetate. The combined organic layers were washed with brine, dried over anhydrous magnesium sulfate. After removal of the solvent, the residue was purified using column chromatography (20% EtOAc-Hexane) to give 2-(3-phenyl-[1,2,4]triazol-1-yl)-pyridine (238 mg, 71%). [M+H]⁺ 223.2; 1H NMR (400 MHz, DMSO-D6) δ ppm 5.73 (d, J=0.73 Hz, 1 H) 7.44-7.55 (m, 4 H) 7.92-7.97 (m, 1 H) 8.06-8.14 (m, 3 H) 8.50-8.58 (m, 1 H) 9.40 (s, 1 H).

Compound 631: 4-Bromo-2-(1-pyridin-2-yl-1H-[1,2,4]triazol-3-yl)-phenol (Example 317)

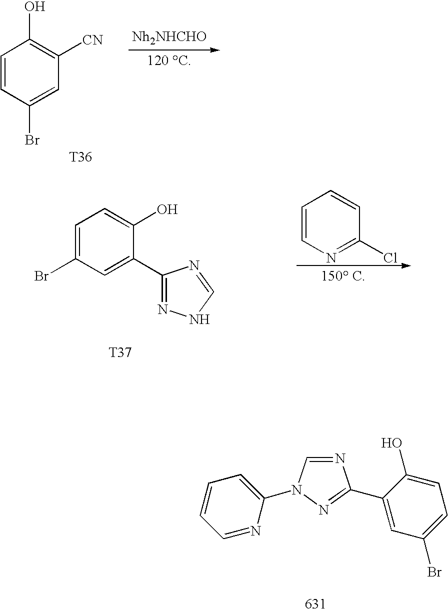

Step 1: Preparation of 4-Bromo-2-(1H-[1.2,4]triazol-3-yl)-phenol

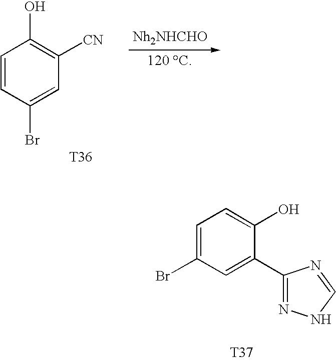

A mixture of 5-bromo-2-hydroxy-benzonitrile (1.97 g, 10 mmol) and formic acid hydrazide (0.6 g, 10 mmol) was purged with argon for 5 min and heated in a sealed tube at 120° C. for 1 hr. The reaction crude was purified using column chromatography (20% EtOAc-Hexane) to give 4-bromo-2-(1H-[1,2,4]triazol-3-yl)-phenol (980 mg, 41%). [M+H]⁺ 240.2; 1H NMR (400 MHz, DMSO-D6) δ ppm 6.86-7.03 (m, 2 H) 7.45 (dd, J=8.80, 2.20 Hz, 1 H) 7.62 (dd, J=9.16, 2.57 Hz, 1 H) 7.83 (d, J=2.57 Hz, 1 H) 8.05 (s, 1 H) 8.81 (s, 1 H) 11.35 (s, 2 H).

Step 2

Compound 631: 4-Bromo-2-(1-pyridin-2-yl-1H-[1,2,4]triazol-3-yl)-phenol

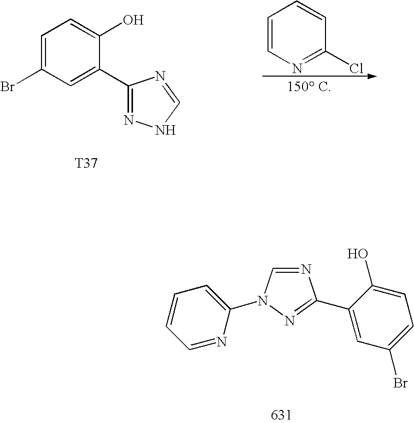

A mixture of 4-bromo-2-(1H-[1,2,4]triazol-3-yl)-phenol (239 mg, 1.0 mmol) and 2-chloropyridine (170 mg, 1.5 mmol) was purged with argon for 5 min and heated in a sealed tube at 130° C. for 24 hr. The reaction mixture was partitioned between water and ethyl acetate. The aqueous layer was separated and extracted three times with ethyl acetate. The combined organic layers were washed with brine, dried over anhydrous magnesium sulfate. After removal of the solvent, the residue was purified using column chromatography (0-5% EtOAc-Hexane) to give 4-bromo-2-(1-pyridin-2-yl-1H-[1,2,4]triazol-3-yl)-phenol (180 mg, 57%). [M+H]⁺ 317.0; 1H NMR (400 MHz, DMSO-D6) δ ppm 7.00 (d, J=8.80 Hz, 1 H) 7.48-7.55 (m, 2 H) 8.03-8.14 (m, 3 H) 8.54-8.60 (m, 1 H) 9.64 (s, 1 H) 10.71 (s, 1 H).

Compound 635: 4-Bromo-2-[1-(3-methyl-pyridin-2-yl)-1H-[1,2,4]triazol-3-yl]-phenol (Example 318)

4-Bromo-2-[1-(3-methyl-pyridin-2-yl)-1H-[1,2,4]triazol-3-yl]-phenol was prepared from 4-bromo-2-(1H-[1,2,4]triazol-3-yl)-phenol and 2-chloro3-methyl-pyridine using the protocol described in Example 317, step 2. [M+H]⁺ 331.3; 1H NMR (400 MHz, DMSO-D6) δ ppm 3.30 (s, 3 H) 7.00 (d, J=8.58 Hz, 1 H) 7.50 (dd, J=8.78, 2.54 Hz, 1 H) 7.54 (dd, J=7.80, 4.68 Hz, 1 H) 7.99 (dd, J=8.00, 2.15 Hz, 1 H) 8.07 (d, J=2.73 Hz, 1 H) 8.45 (dd, J=4.29, 1.56 Hz, 1 H) 9.38 (s, 1 H) 10.78 (s, 1 H).

Compound 634: 4-Bromo-2-[1-(3-trifluoromethyl-pyridin-2-yl)-1H-[1,2,4]triazol-3-yl]-phenol (Example 319)

4-Bromo-2-[1-(3-trifluoromethyl-pyridin-2-yl)-1H-[1,2,4]triazol-3-yl]-phenol was prepared from 4-bromo-2-(1H-[1,2,4]triazol-3-yl)-phenol and 2-chloro-3-trifluoromethyl-pyridine using the protocol described in Example 317, step 2. [M+H]⁺ 385.1; 1H NMR (400 MHz, DMSO-D6) δ ppm 7.01 (d, J=8.97 Hz, 1 H) 7.52 (dd, J=8.78, 2.54 Hz, 1 H) 7.90 (dd, J=8.39, 4.49 Hz, 1 H) 8.03 (d, J=2.34 Hz, 1 H) 8.59 (dd, J=8.19, 1.56 Hz, 1 H) 8.93 (dd, J=4.68, 1.56 Hz, 1 H) 9.48 (s, 1 H) 10.42 (s, 1 H).

Compound 629: 2-(1-Pyridin-2-yl-1H-[1,2,4]triazol-3-yl)-phenol (Example 320)

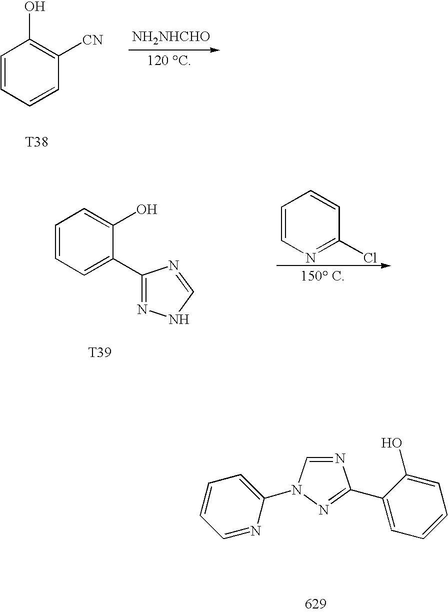

Step 1: Preparation of 2-(1H-[1,2,4]Triazol-3-yl)-phenol

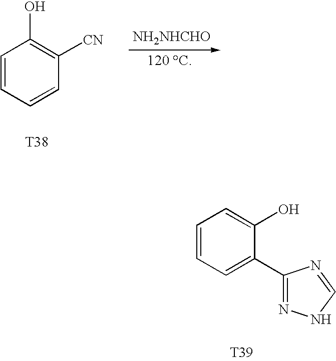

A mixture of 2-hydroxy-benzonitrile (1.19 g, 10 mmol) and formic acid hydrazide ( 0.6 g, 10 mmol) was purged with argon for 5 min and heated in a sealed tube at 120° C. for 1 hr. The reaction crude was used without further purification. [M+H]⁺ 162.2.

Step 2

Compound 629: Preparation 2-(1-Pyridin-2-yl-1H-[1,2,4]triazol-3-yl)-phenol

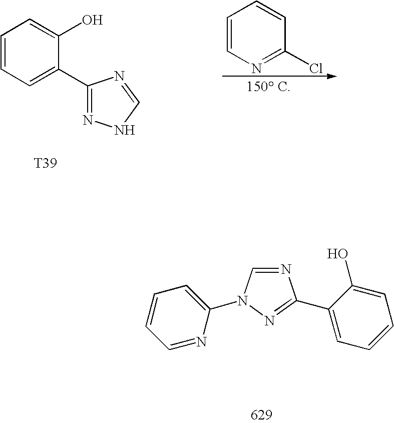

A mixture of 2-(1H-[1,2,4]triazol-3-yl)-phenol ( 161 mg, 1.0 mmol) and 2-chloropyridine (170 mg, 1.5 mmol) was purged with argon for 5 min and heated in a sealed tube at 130° C. for 24 hr. The reaction mixture was purified using preperative LC/MS to give 2-(1-Pyridin-2-yl-1H-[1,2,4]triazol-3-yl)-phenol. [M+H]⁺ 239.2. ¹H (CDCl₃), 1H NMR (400 MHz, DMSO-D6) δ ppm 6.96-7.03 (m, 2 H) 7.33-7.39 (m, 1 H) 7.50-7.55 (m, 1 H) 7.99-8.07 (m, 2 H) 8.08-8.14 (m, 1 H) 8.55-8.59 (m, 1 H) 9.61 (s, 1 H) 10.65 (s, 1 H).

Compound 630: 4-Chloro-2-(1-pyridin-2-yl-1H-[1,2,4]triazol-3-yl)-phenol (Example 321)

Step 1: Preparation of 4-chloro-2-( l H-f 1,2,41triazol-3-yl)-phenol

4-Chloro-2-(1H-[1,2,4]triazol-3-yl)-phenol was prepared from 5-chloro-2-hydroxy-benzonitrile and formic acid hydrazide using the protocol described in Example 320, step 1. The reaction crude was used without fuirther purification. [M+H]⁺ 196.1.

Step 2: Preparation of 4-chloro-2-(1-pyridin-2-yl-1H-[1,2,4]triazol-3-yl)-phenol

4-Chloro-2-(1-pyridin-2-yl-1H-[1,2,4]triazol-3-yl)-phenol was prepared from 4-chloro-2-(1H-[1,2,4]triazol-3-yl)-phenol and 2-chloropyridine using the protocol described in Example 320, step 2. [M+H]⁺ 273.2; 1H NMR (400 MHz, DMSO-D6) δ ppm 7.05 (d, J=8.80 Hz, 1 H) 7.40 (dd, J=8.80, 2.93 Hz, 1 H) 7.49-7.56 (m, 1 H) 7.96-8.15 (m, 3 H) 8.55-8.60 (m, 1 H) 9.64 (s, 1 H) 10.69 (s, 1 H).

Compound 639: 3-Pyridinyl-[1,2,4]triazol-1-yl)-pyridine (Example 322)

Step 1: Preparation of 2-(1H-[1,2,4]Triazol-3-yl)-pyridine

2-(1H-[1,2,4]Triazol-3-yl)-pyridine was prepared from Pyridine-2-carbonitrile and formic acid hydrazide using the protocol described in Example 320, step 1. The reaction crude was used without further purification. [M+H]⁺ 147.2

Step 2: Preparation of 3-pyridinyl-[1,2,4]triazol-1-yl)-pyridine

3-Pyridinyl-[1,2,4]triazol-1-yl)-pyridine was prepared from 2-(1H-[1,2,4]Triazol-3-yl)-pyridine and 2-chloropyridine using the protocol described in Example 320, step 2. [M+H]⁺ 224.2; 1H NMR (400 MHz, DMSO-D6) δ ppm 7.45-7.54 (m, 2 H) 7.92-8.00 (m, 2 H) 8.07-8.19 (m, 2 H) 8.53-8.59 (m, 1 H) 8.68-8.73 (m, 1 H) 9.46 (s, 1 H).

Compound 636: 4-Methoxy-2-(1-pyridin-2-yl-1H-[1,2,4]triazol-3-yl)-phenol (Example 323)

Step 1: Preparation of 4-methoxy-2-(1H-[1,2,4]triazol-3-yl)-phenol

4-Methoxy-2-(1H-[1,2,4]triazol-3-yl)-phenol was prepared from 5-methoxy-2-hydroxy-benzonitrile and formic acid hydrazide using the protocol described in Example 320, step 1. The reaction crude was used without further purification. [M+H]⁺ 192.2.

Step 2: Preparation of 4-Methoxy-2-(1-pyridin-2-yl-1H-[1,2,4]triazol-3-yl)-phenol

4-Methoxy-2-(1-pyridin-2-yl-1H-[1,2,4]triazol-3-yl)-phenol was prepared from 4-Methoxy-2-(1H-[1,2,4]triazol-3-yl)-phenol and 2-chloropyridine using the protocol described in Example 320, step 2. [M+H]⁺ 269.2; 1H NMR (400 MHz, DMSO-D6) δ ppm 3.78 (s, 3 H) 7.48-7.59 (m, 2 H) 8.00-8.07 (m, 1 H) 8.08-8.16 (m, 1 H) 8.51-8.63 (m, 1 H) 9.62 (s, 1 H) 10.22 (s, 1 H).

Compound 637: 4-Methoxy-2-[1-(3-methyl-pyridin-2-yl)-1H-[1,2,4]triazol-3-yl]-phenol (Example 324)

4-Methoxy-2-[1-(3-methyl-pyridin-2-yl)-1H-[1,2,4]triazol-3-yl]-phenol was prepared from 4-Methoxy-2-(1H-[1,2,4]triazol-3-yl)-phenol and 2-chloro3-methyl-pyridine using the protocol described in Example 320, step 2. [M+H]⁺ 283.1; 1H NMR (400 MHz, DMSO-D6) δ ppm 2.48 (s, 3 H) 3.75 (s, 3 H) 6.93-6.98 (m, 2 H) 7.49 (d, J=2.73 Hz, 1 H) 7.54 (dd, J=7.61, 4.88 Hz, 1 H) 7.99 (dd, J=8.00, 1.36 Hz, 1 H) 8.45 (dd, J=5.07, 1.56 Hz, 1 H) 9.35 (s, 1 H) 10.26 (s, 1 H).

Compound 638: 4-Methoxy-2-[1-(3-trifluoromethyl-pyridin-2-yl)-1H-[1,2,4]triazol-3-yl]-phenol (Example 325)

4-Methoxy-2-[1-(3-trifluoromethyl-pyridin-2-yl)-1H-[1,2,4]triazol-3-yl]-phenol was prepared from 4-Methoxy-2-(1H-[1,2,4]triazol-3-yl)-phenol and 2-chloro-3-trifluoromethyl-pyridine using the protocol described in Example 320, step 2. [M+H]⁺ 337.2.

Exemplary compounds are shown in the Table.

Biological Testing

Any method suitable for measuring HCV replication and/or infectivity can be used to evaluate the compounds of the invention. In vitro methods are particularly suitable for testing large numbers of compounds, but in vivo methods are also suitable.

The ability of compounds of Formulas I to inhibit HCV replication was demonstrated in the following two cell-based in vitro assays. The results of the EC₅₀ assay are summarized in the Table by compound number.

HCV Replicon Assays (Replicon EC₅₀ (μM))

HCV replication can be measured by a replicon assay such as described by Vrolijk et al, J. Virol. Methods 110:201(2003), which is hereby incorporated in its entirety by reference. The method was implemented as follows.

Luciferase Replicon Reporter Cell Line:

Human hepatocyte Huh7 cells containing the HCV luciferase reporter replicon were obtained from Ralf Bartenschlager at the University of Mainz, Germany. These cells were maintained under neomycin selection and passaged when 80-90% confluent.

This cell line contains an autonomously replicating RNA element (replicon) incorporating the non-structural HCV elements necessary for replication, and upon which the survival of the replicon in the cell depends. The replicon also encodes the synthesis of firefly luciferase. Inhibition of any of the critical HCV functions by a compound leads to loss of the replicon and subsequent loss of luciferase. The amount of luciferase remaining following a standard incubation with a compound is a measure of the anti-HCV activity of the compound.

The assay was conducted by preparing sufficient 96-well plates containing these cells, wherein cells were seeded at 6000 cells/well in a 96-well plate, in 200 μl of final media volume. Cells were then incubated for 24 hours at 37° C., 5% CO₂, and 95% humidity before compound was added.

Six point half-log concentration response assays were conducted to determine potency/EC₅₀ of Formula I compounds to inhibit HCV replicon replication. The final percent DMSO acceptable in this assay system was 0.6%. Compounds were diluted in media in an appropriate format and 5 μl of each drug dilution was added to each well. Cells were then incubated with compounds for 3 days at 37° C., 5% CO₂, and 95% humidity.

Following this incubation period, plates were washed twice with phosphate buffered saline to remove media, and cells were lysed by the addition of 25 il of lx Passive Lysis Buffer (Promega Cat#E1941) to each well, and gently shaken at room temperature for 20 minutes. The luciferase activity in each well was then determined, and the inhibition calculated by reference to appropriate controls.

Monocistronic Replicon Cell Line:

Human hepatocyte Huh7 cells containing the monocistronic NK5.1 replicon were obtained from Ralf Bartenschlager at the University of Mainz, Germany. These cells were maintained under hygromycin selection and passaged when 80-90% confluent.

This cell line contains an autonomously replicating RNA element (replicon) incorporating the non-structural HCV elements necessary for replication, and upon which the survival of the replicon in the cell depends. This replicon contains a single internal ribosome entry site (HCV IRES). Inhibition of any of the critical HCV finctions by a compound leads to loss of the replicon as measured by HCV replicon RNA copy number reduction. The amount of HCV RNA present following a standard incubation with a compound is a measure of the anti-HCV activity of the compound. The assay was conducted by preparing sufficient 96-well plates containing these cells, wherein cells are seeded at 6000 cells/well in a 96-well plate, in 200 μl of final media volume. Cells were then incubated for 24 hours at 37 C., 5% CO₂, and 95% humidity before compound was added. Six point half-log concentration response assays were conducted to determine potency/EC₅₀ of Formula I compounds to inhibit HCV replicon replication. The final percent DMSO acceptable in this assay system is 0.6%. Compounds were diluted in media in an appropriate format and 5 μl of each drug dilution is added to each well. Cells were then incubated with compounds for 3 days at 37 C., 5% CO₂, and 95% humidity. Following the three day incubation period, plates were washed twice with phosphate buffered saline to remove media and cells were lysed with Qiagen extraction buffer. RNA was extracted and isolated with the Qiagen RNAeasy kit according to the manufacturer's protocol (Qiagen). TaqMan One-Step RT-PCR was used in conjunction with an ABI 7700 Sequence Detector (Applied Biosystems) for the quantitation and analysis of HCV replicon RNA copy number. A GAPDH probe was used for internal normalization. The ratio of HCV RNA to cellular GAPDH RNA was determined and compared to the DMSO-only treated control, and the ratio of HCV RNA/GAPDH RNA was used for an EC₅₀ determination and as a measure of specificity for inhibition of the HCV replicon.

HCV Cell Cytotoxicity Assay (Replicon CC₅₀ (μM))

Cell toxicity assays for CC₅₀ determination were conducted on Huh7 cells in a protocol parallel to the HCV replicon assay described above. However, rather than measuring the luciferase activity, cell number was measured using XTT (Sigma) and detected with a fluorometer at 450 nM/650 nM using Genios/Tecan (Xfluor). For these studies, cells were incubated in 96 well microtiter plates for 3 days with several concentrations of each compound, wherein the concentrations encompassed the range previously found to be effective in inhibiting HCV replication. At the end of that time, the cells were treated with an XTT solution and toxicity measured and CC₅₀ determinations automatically recorded.

TABLE 1

| | | | Mono EC50 |
|---|---|---|---|
| | Structure | Class | Median [uM] |
| 1 | 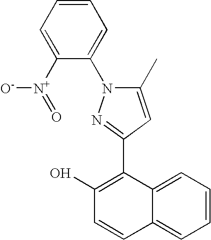 | Pyrazoles | >32 |
| 2 | 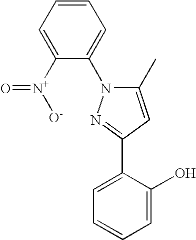 | Pyrazoles | >32 |
| 3 | 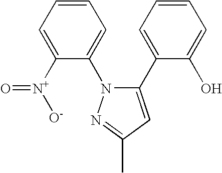 | Pyrazoles | 31 |
| 4 | 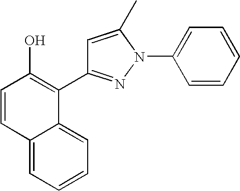 | Pyrazoles | >19 |
| 5 | 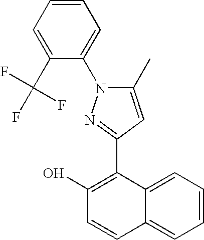 | Pyrazoles | >19 |
| 6 | 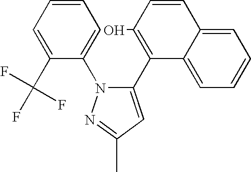 | Pyrazoles | 13 |
| 7 | 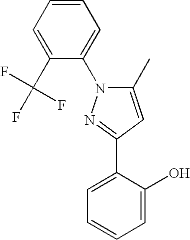 | Pyrazoles | 20 |
| 8 | 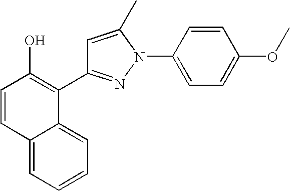 | Pyrazoles | >19 |
| 9 | 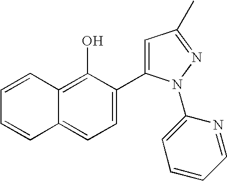 | Pyrazoles | >19 |
| 10 | 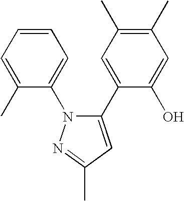 | Pyrazoles | >19 |
| 11 | 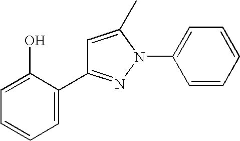 | Pyrazoles | 9 |
| 12 | 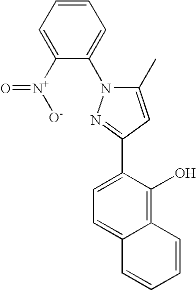 | Pyrazoles | 13 |
| 13 | 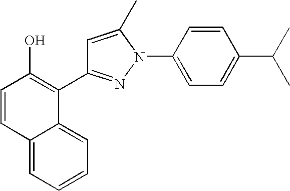 | Pyrazoles | >19 |
| 14 | 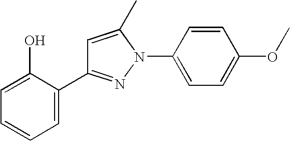 | Pyrazoles | 11 |
| 15 | 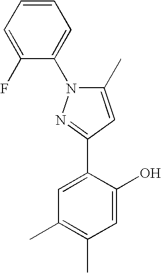 | Pyrazoles | >19 |
| 16 | 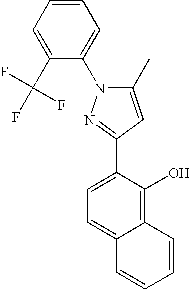 | Pyrazoles | 17 |
| 17 | 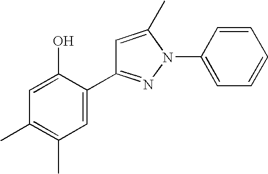 | Pyrazoles | >19 |
| 18 | 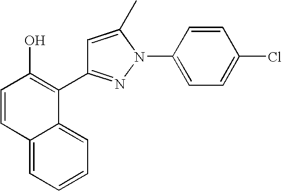 | Pyrazoles | 18 |
| 19 | 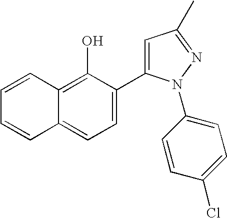 | Pyrazoles | 17 |
| 20 | 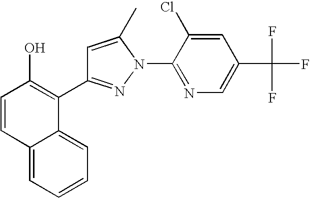 | Pyrazoles | 13 |
| 21 | 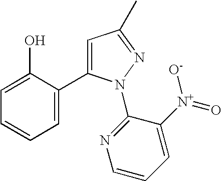 | Pyrazoles | >19 |
| 22 | 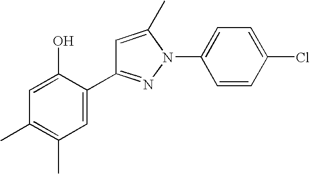 | Pyrazoles | >19 |
| 23 | 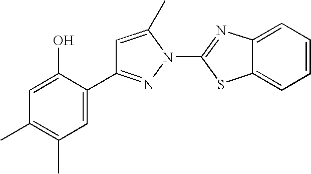 | Pyrazoles | >19 |
| 24 | 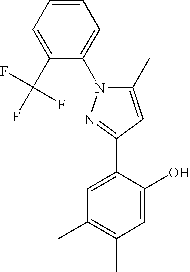 | Pyrazoles | >19 |
| 25 | 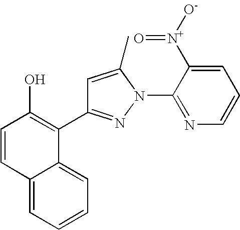 | Pyrazoles | >19 |
| 26 | 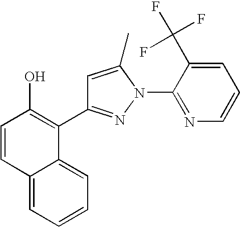 | Pyrazoles | >19 |
| 27 | 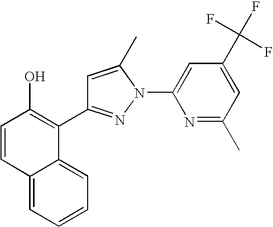 | Pyrazoles | 12 |
| 28 | 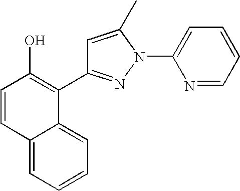 | Pyrazoles | >19 |
| 29 | 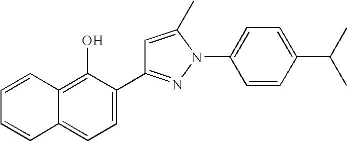 | Pyrazoles | >19 |
| 30 | 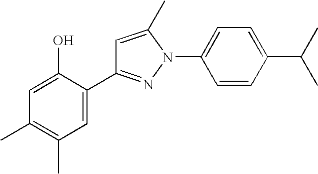 | Pyrazoles | 12 |
| 31 | 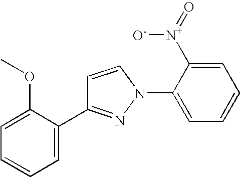 | Pyrazoles | 21 |
| 32 | 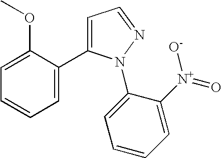 | Pyrazoles | 3.6 |
| 33 | 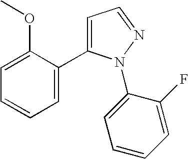 | Pyrazoles | 27 |
| 34 | 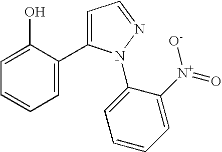 | Pyrazoles | 5.3 |
| 35 | 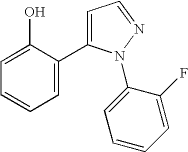 | Pyrazoles | 14 |
| 36 | 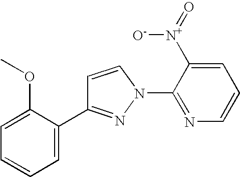 | Pyrazoles | 2.3 |
| 37 | 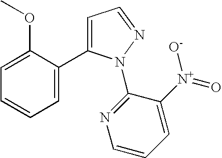 | Pyrazoles | >32 |
| 38 | 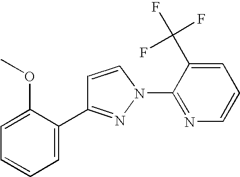 | Pyrazoles | 13 |
| 39 | 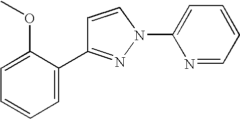 | Pyrazoles | >32 |
| 40 | 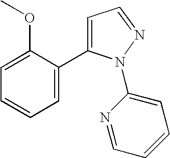 | Pyrazoles | >32 |
| 41 | 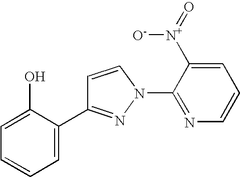 | Pyrazoles | 6 |
| 42 | 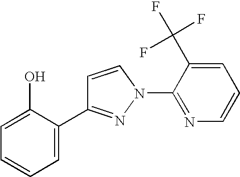 | Pyrazoles | 6 |
| 43 | 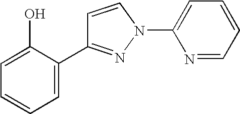 | Pyrazoles | 15 |
| 44 | 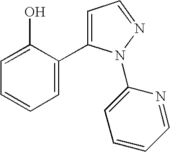 | Pyrazoles | >32 |
| 45 | 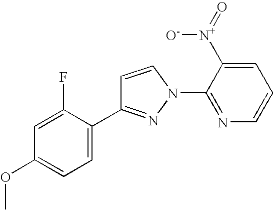 | Pyrazoles | 5.8 |
| 46 | 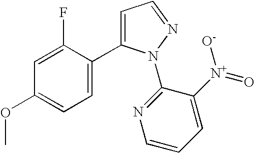 | Pyrazoles | 6 |
| 47 | 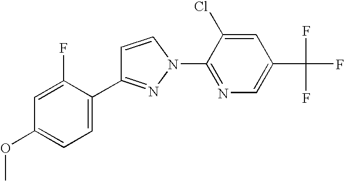 | Pyrazoles | 6 |
| 48 | 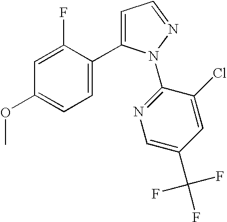 | Pyrazoles | 15 |
| 49 | 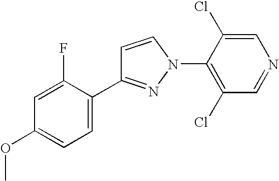 | Pyrazoles | >32 |
| 50 | 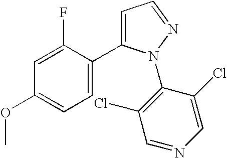 | Pyrazoles | 5.8 |
| 51 | 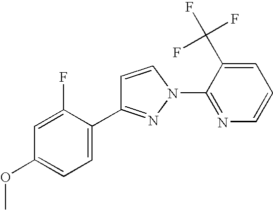 | Pyrazoles | 20 |
| 52 | 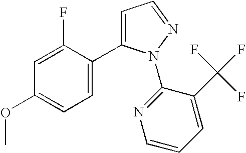 | Pyrazoles | 2.1 |
| 53 | 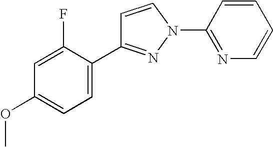 | Pyrazoles | >32 |
| 54 | 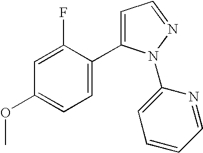 | Pyrazoles | 2.2 |
| 55 | 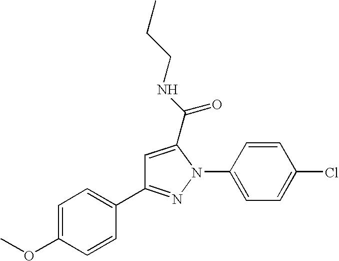 | Pyrazoles | 18 |
| 56 | 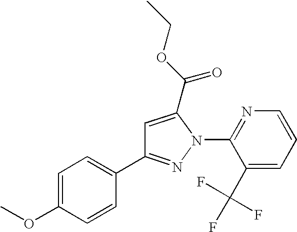 | Pyrazoles | >32 |
| 57 | 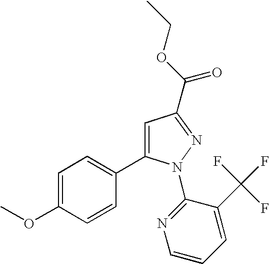 | Pyrazoles | >32 |
| 58 | 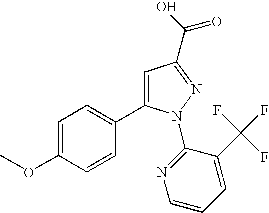 | Pyrazoles | >32 |
| 59 | 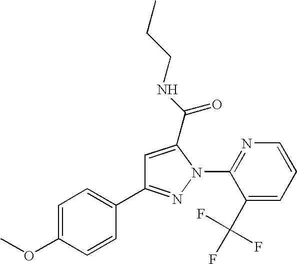 | Pyrazoles | >32 |
| 60 | 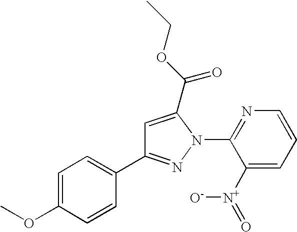 | Pyrazoles | >32 |
| 61 | 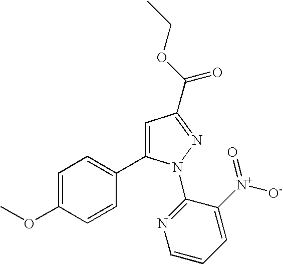 | Pyrazoles | 19 |
| 62 | 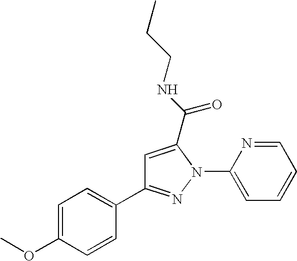 | Pyrazoles | >32 |
| 63 | 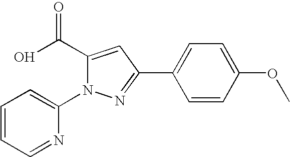 | Pyrazoles | >32 |
| 64 | 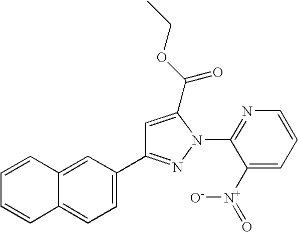 | Pyrazoles | >32 |
| 65 | 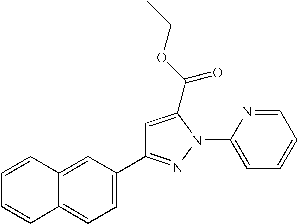 | Pyrazoles | >32 |
| 66 | 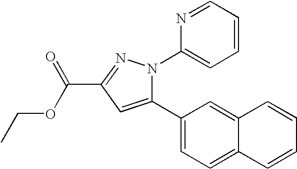 | Pyrazoles | 30 |
| 67 | 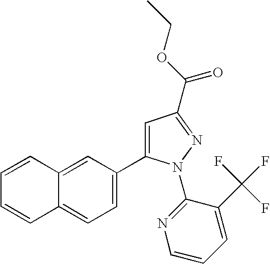 | Pyrazoles | 22 |
| 68 | 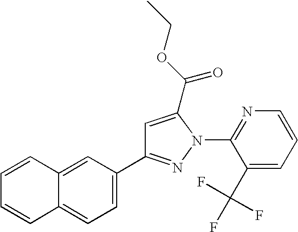 | Pyrazoles | 12 |
| 69 | 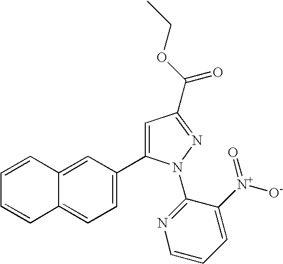 | Pyrazoles | 24 |
| 70 | 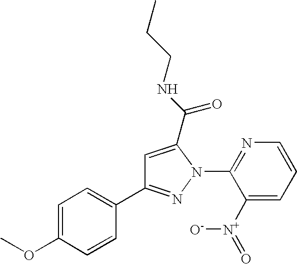 | Pyrazoles | >32 |
| 71 | 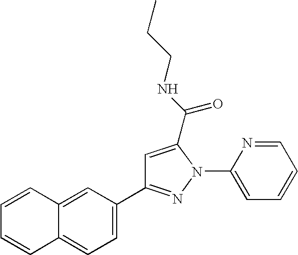 | Pyrazoles | 21 |
| 72 | 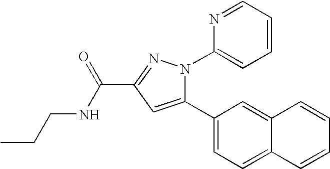 | Pyrazoles | >32 |
| 73 | 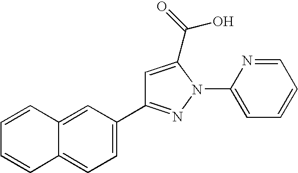 | Pyrazoles | >32 |
| 74 | 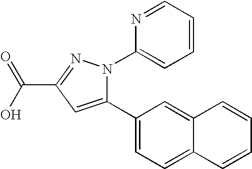 | Pyrazoles | >32 |
| 75 | 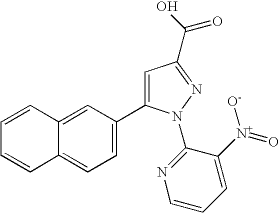 | Pyrazoles | >32 |
| 76 | 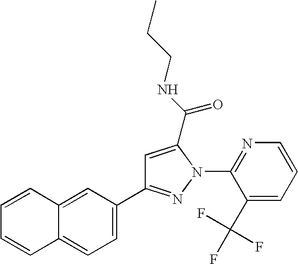 | Pyrazoles | 9.4 |
| 77 | 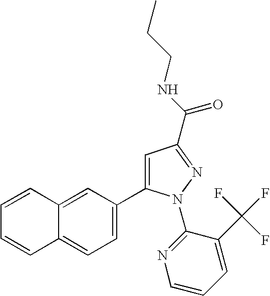 | Pyrazoles | >32 |
| 78 | 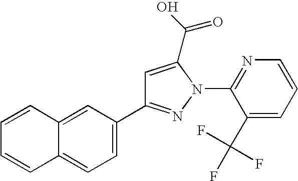 | Pyrazoles | >32 |
| 79 | 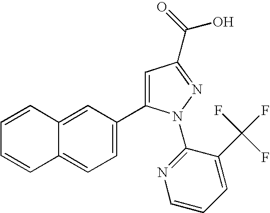 | Pyrazoles | >32 |
| 80 | 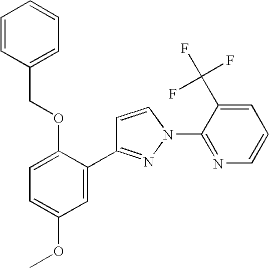 | Pyrazoles | 1.8 |
| 81 | 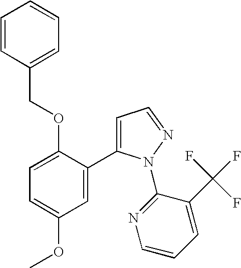 | Pyrazoles | 6.2 |
| 82 | 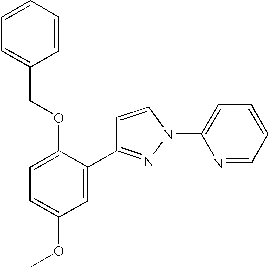 | Pyrazoles | 4.3 |
| 83 | 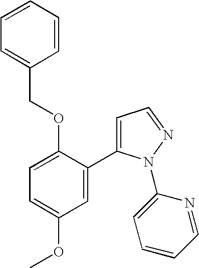 | Pyrazoles | 31 |
| 84 | 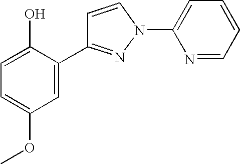 | Pyrazoles | 3.3 |
| 85 | 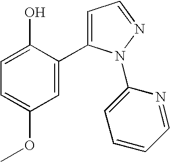 | Pyrazoles | >32 |
| 86 | 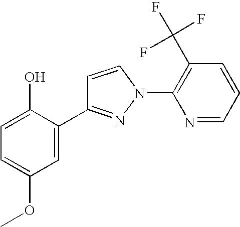 | Pyrazoles | 7.8 |
| 87 | 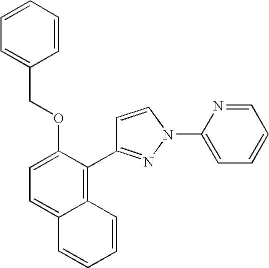 | Pyrazoles | >32 |
| 88 | 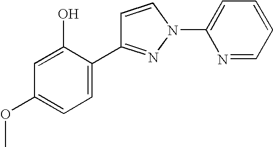 | Pyrazoles | >32 |
| 89 | 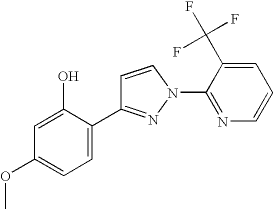 | Pyrazoles | 8.8 |
| 90 | 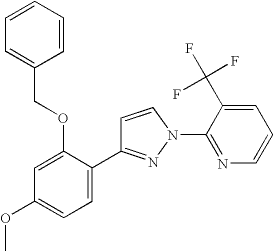 | Pyrazoles | 4.4 |
| 91 | 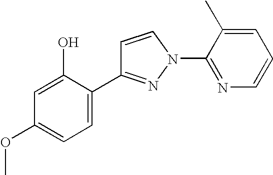 | Pyrazoles | 9.1 |
| 92 | 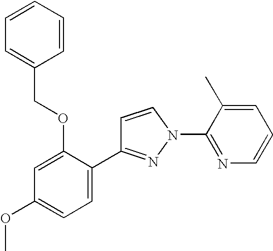 | Pyrazoles | 5.2 |
| 93 | 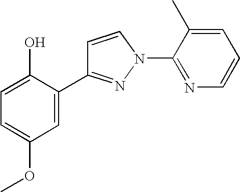 | Pyrazoles | 9.1 |
| 94 | 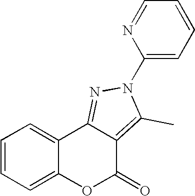 | Pyrazoles | 8.9 |
| 95 | 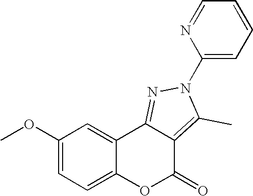 | Pyrazoles | 5.8 |
| 96 | 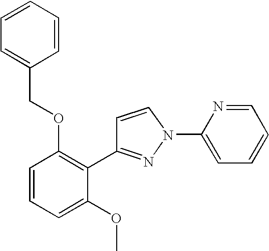 | Pyrazoles | 8.6 |
| 97 | 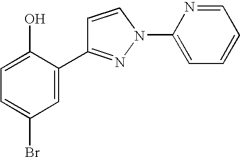 | Pyrazoles | 22 |
| 98 | 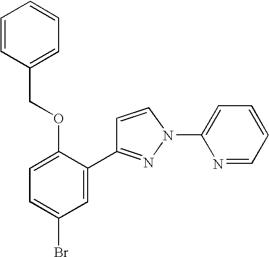 | Pyrazoles | 15 |
| 99 | 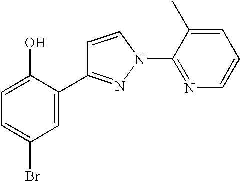 | Pyrazoles | 3.6 |
| 100 | 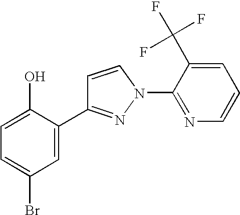 | Pyrazoles | 2.3 |
| 101 | 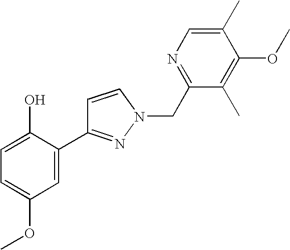 | Pyrazoles | 14.6 |
| 102 | 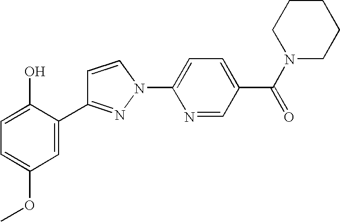 | Pyrazoles | >32 |
| 103 | 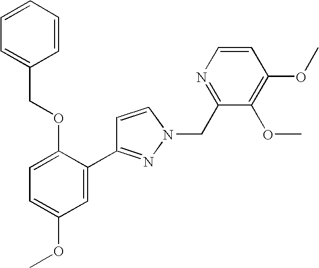 | Pyrazoles | >32 |
| 104 | 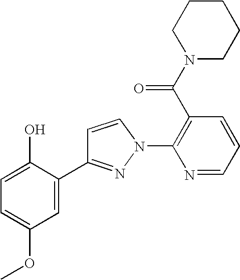 | Pyrazoles | >32 |
| 105 | 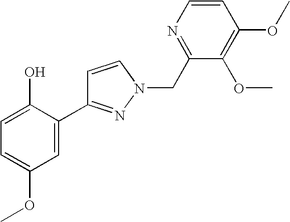 | Pyrazoles | >32 |
| 106 | 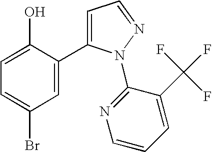 | Pyrazoles | 15 |
| 107 | 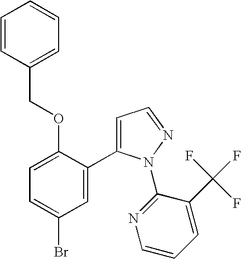 | Pyrazoles | 2.6 |
| 108 | 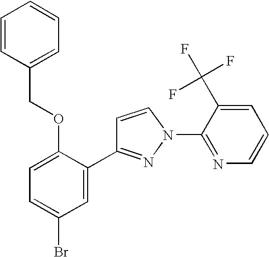 | Pyrazoles | >32 |
| 109 | 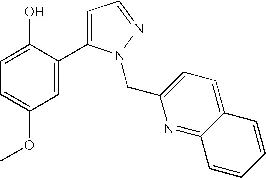 | Pyrazoles | >32 |
| 110 | 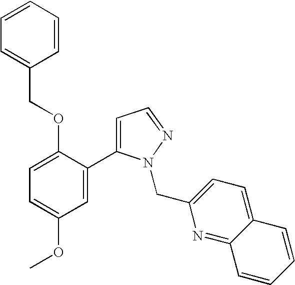 | Pyrazoles | 8 |
| 111 | 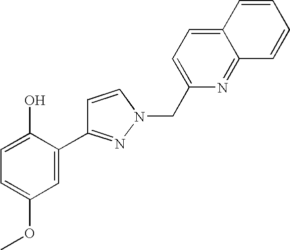 | Pyrazoles | 14 |
| 112 | 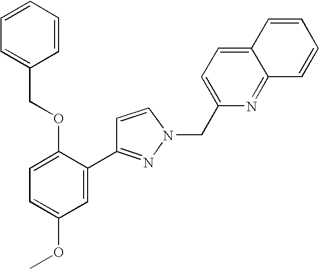 | Pyrazoles | 6 |
| 113 | 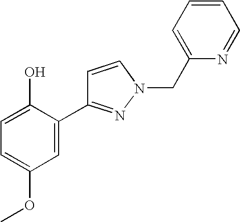 | Pyrazoles | >32 |
| 114 | 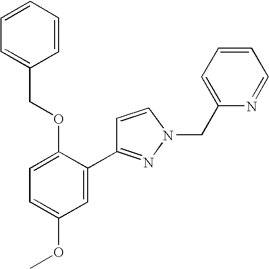 | Pyrazoles | 9.5 |
| 115 | 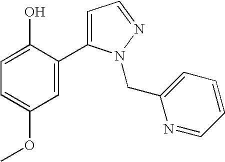 | Pyrazoles | >32 |
| 116 | 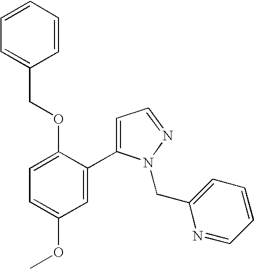 | Pyrazoles | 13 |
| 117 | 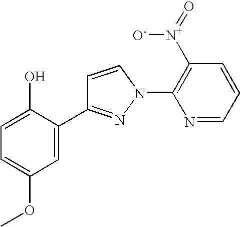 | Pyrazoles | 25.4 |
| 118 | 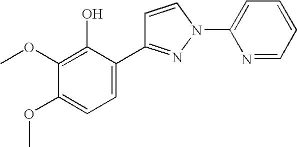 | Pyrazoles | 23.8 |
| 119 | 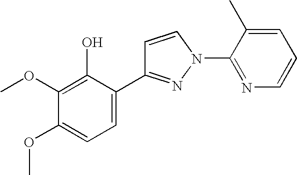 | Pyrazoles | 11.8 |
| 120 | 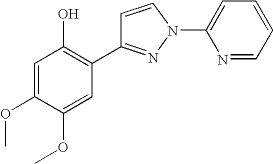 | Pyrazoles | 31.2 |
| 121 | 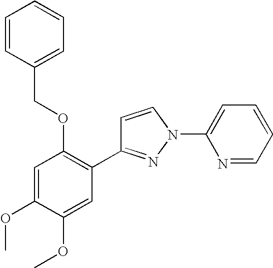 | Pyrazoles | 13.5 |
| 122 | 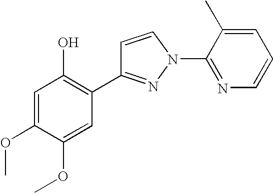 | Pyrazoles | 13 |
| 123 | 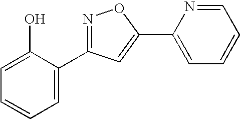 | Isoxazoles | >32 |
| 124 | 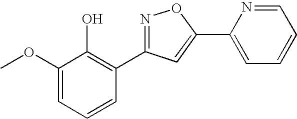 | Isoxazoles | 26 |
| 125 | 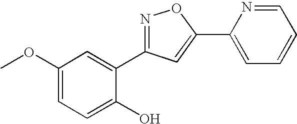 | Isoxazoles | 11 |
| 126 | 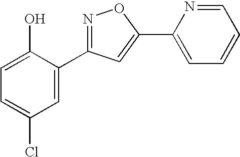 | Isoxazoles | >32 |
| 127 | 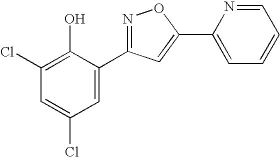 | Isoxazoles | 7.4 |
| 128 | 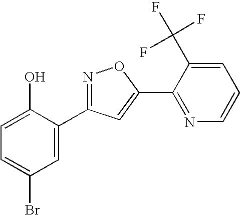 | Isoxazoles | >32 |
| 129 | 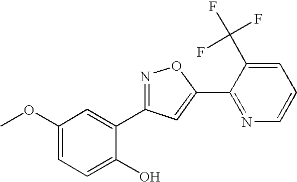 | Isoxazoles | 3.8 |
| 130 | 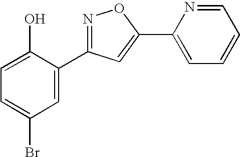 | Isoxazoles | 11 |
| 131 | 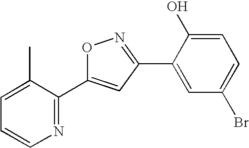 | Isoxazoles | 4.5 |
| 132 | 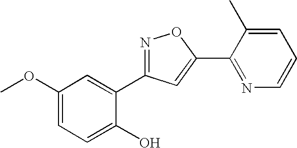 | Isoxazoles | 2 |
| 133 | 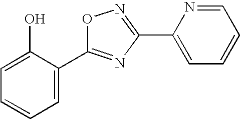 | 1,2,4 Oxadiazoles | >19 |
| 134 | 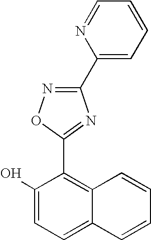 | 1,2,4 Oxadiazoles | 17 |
| 135 | 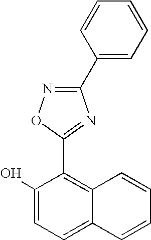 | 1,2,4 Oxadiazoles | >32 |
| 136 | 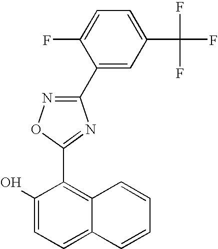 | 1,2,4 Oxadiazoles | >32 |
| 137 | 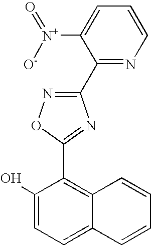 | 1,2,4 Oxadiazoles | 1.8 |
| 138 | 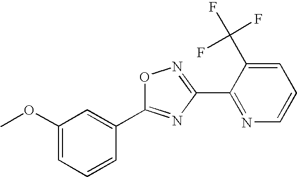 | 1,2,4 Oxadiazoles | >19 |
| 139 | 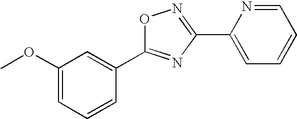 | 1,2,4 Oxadiazoles | 18 |
| 140 | 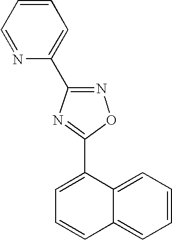 | 1,2,4 Oxadiazoles | 7.1 |
| 141 | 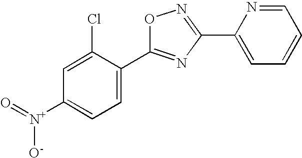 | 1,2,4 Oxadiazoles | 10 |
| 142 | 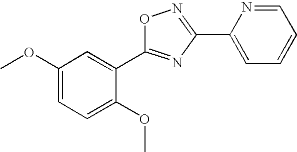 | 1,2,4 Oxadiazoles | 20 |
| 143 | 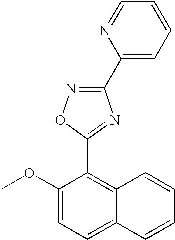 | 1,2,4 Oxadiazoles | 13 |
| 144 | 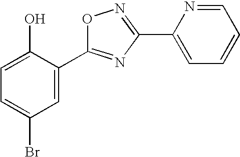 | 1,2,4 Oxadiazoles | 3.7 |
| 145 | 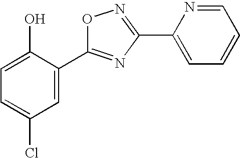 | 1,2,4 Oxadiazoles | 2.3 |
| 146 | 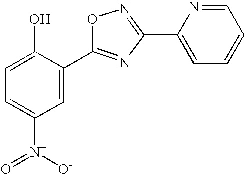 | 1,2,4 Oxadiazoles | >32 |
| 147 | 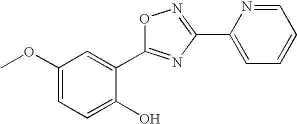 | 1,2,4 Oxadiazoles | 4.4 |
| 148 | 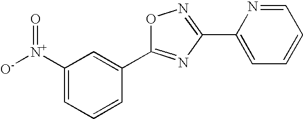 | 1,2,4 Oxadiazoles | >19 |
| 149 | 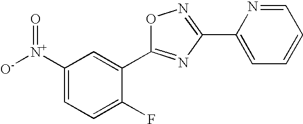 | 1,2,4 Oxadiazoles | 4.1 |
| 150 | 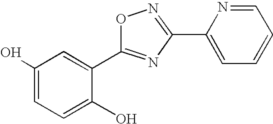 | 1,2,4 Oxadiazoles | 16 |
| 151 | 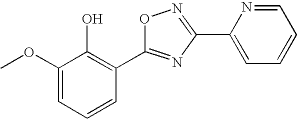 | 1,2,4 Oxadiazoles | 18 |
| 152 | 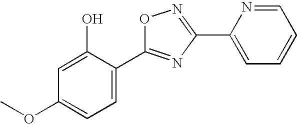 | 1,2,4 Oxadiazoles | 5.6 |
| 153 | 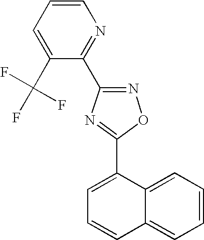 | 1,2,4 Oxadiazoles | 7.9 |
| 154 | 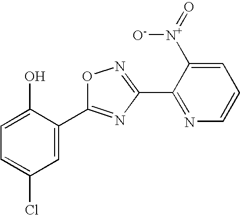 | 1,2,4 Oxadiazoles | >19 |
| 155 | 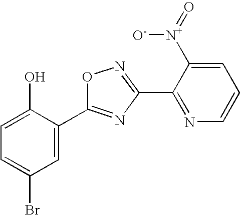 | 1,2,4 Oxadiazoles | >19 |
| 156 | 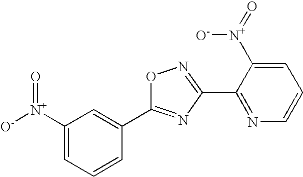 | 1,2,4 Oxadiazoles | >19 |
| 157 | 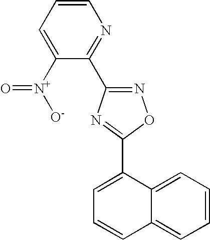 | 1,2,4 Oxadiazoles | 4.7 |
| 158 | 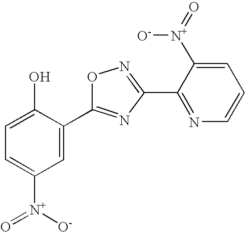 | 1,2,4 Oxadiazoles | 11 |
| 159 | 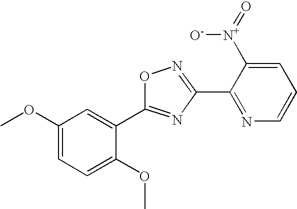 | 1,2,4 Oxadiazoles | 16 |
| 160 | 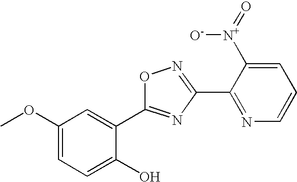 | 1,2,4 Oxadiazoles | >19 |
| 161 | 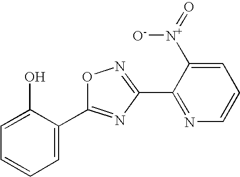 | 1,2,4 Oxadiazoles | >19 |
| 162 | 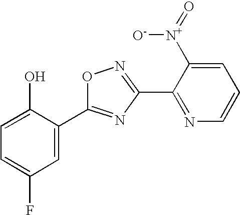 | 1,2,4 Oxadiazoles | 17 |
| 163 | 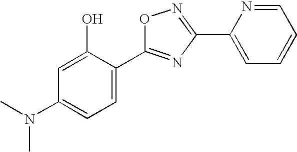 | 1,2,4 Oxadiazoles | 1.5 |
| 164 | 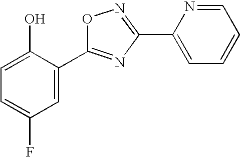 | 1,2,4 Oxadiazoles | 9 |
| 165 | 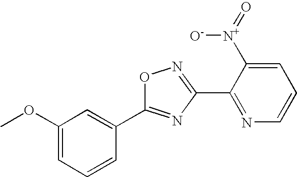 | 1,2,4 Oxadiazoles | 18 |
| 166 | 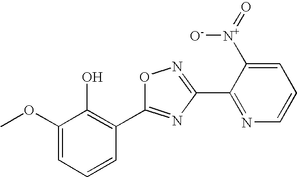 | 1,2,4 Oxadiazoles | >19 |
| 167 | 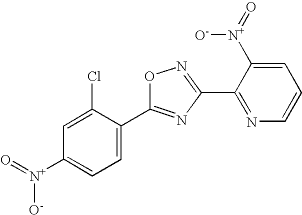 | 1,2,4 Oxadiazoles | 12 |
| 168 | 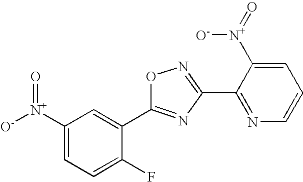 | 1,2,4 Oxadiazoles | 4.6 |
| 169 | 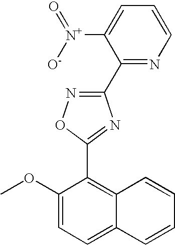 | 1,2,4 Oxadiazoles | >19 |
| 170 | 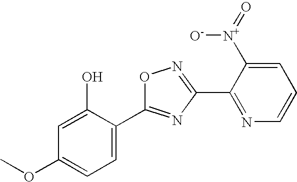 | 1,2,4 Oxadiazoles | 12 |
| 171 | 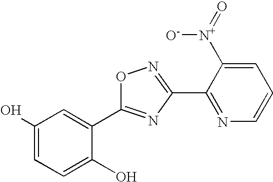 | 1,2,4 Oxadiazoles | 5.4 |
| 172 | 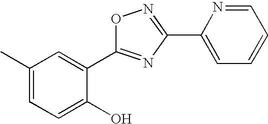 | 1,2,4 Oxadiazoles | 6 |
| 173 | 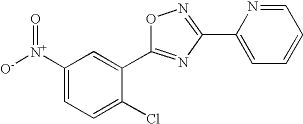 | 1,2,4 Oxadiazoles | 3.8 |
| 174 | 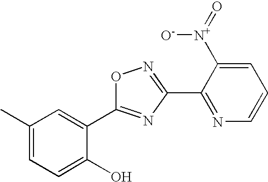 | 1,2,4 Oxadiazoles | 10 |
| 175 | 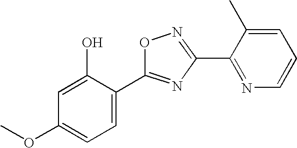 | 1,2,4 Oxadiazoles | 0.5 |
| 176 | 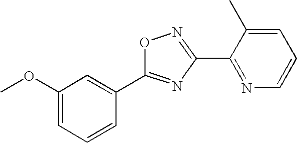 | 1,2,4 Oxadiazoles | 13 |
| 177 | 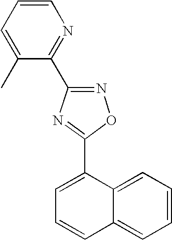 | 1,2,4 Oxadiazoles | 7.2 |
| 178 | 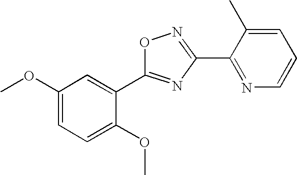 | 1,2,4 Oxadiazoles | >32 |
| 179 | 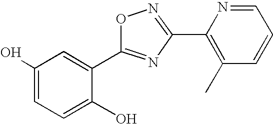 | 1,2,4 Oxadiazoles | 1.3 |
| 180 | 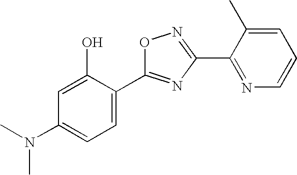 | 1,2,4 Oxadiazoles | 2.1 |
| 181 | 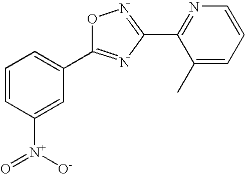 | 1,2,4 Oxadiazoles | >32 |
| 182 | 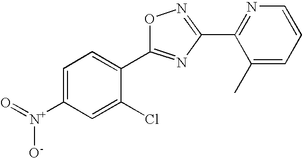 | 1,2,4 Oxadiazoles | 12 |
| 183 | 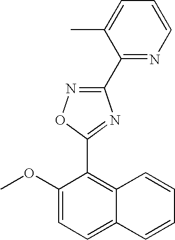 | 1,2,4 Oxadiazoles | >32 |
| 184 | 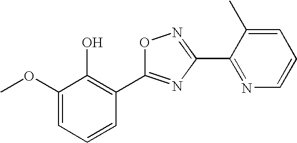 | 1,2,4 Oxadiazoles | 0.68 |
| 185 | 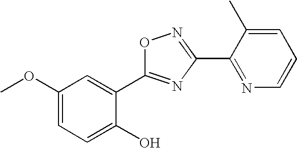 | 1,2,4 Oxadiazoles | 1.6 |
| 186 | 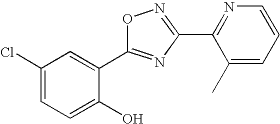 | 1,2,4 Oxadiazoles | 3.9 |
| 187 | 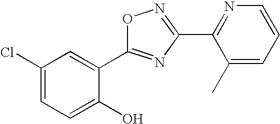 | 1,2,4 Oxadiazoles | 6.5 |
| 188 | 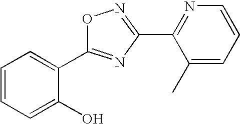 | 1,2,4 Oxadiazoles | >32 |
| 189 | 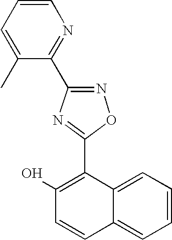 | 1,2,4 Oxadiazoles | 1.2 |
| 190 | 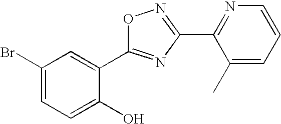 | 1,2,4 Oxadiazoles | 0.69 |
| 191 | 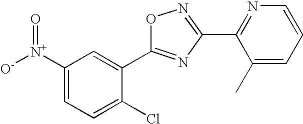 | 1,2,4 Oxadiazoles | 2 |
| 192 | 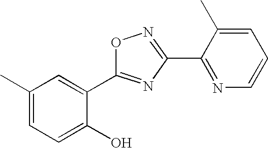 | 1,2,4 Oxadiazoles | >32 |
| 193 | 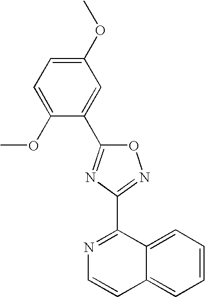 | 1,2,4 Oxadiazoles | 1.3 |
| 194 | 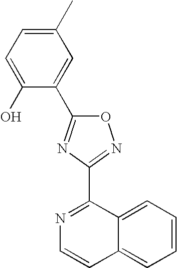 | 1,2,4 Oxadiazoles | >10 |
| 195 | 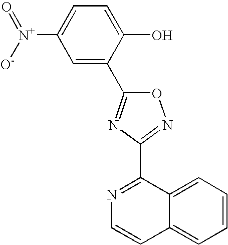 | 1,2,4 Oxadiazoles | >32 |
| 196 | 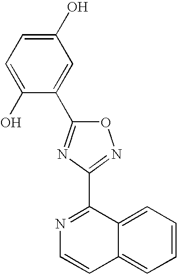 | 1,2,4 Oxadiazoles | >32 |
| 197 | 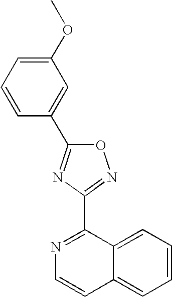 | 1,2,4 Oxadiazoles | >32 |
| 198 | 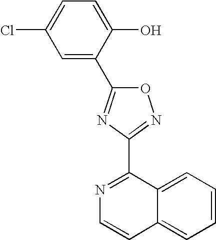 | 1,2,4 Oxadiazoles | >32 |
| 199 | 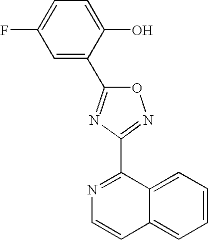 | 1,2,4 Oxadiazoles | 1.1 |
| 200 | 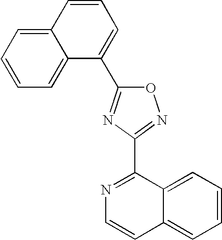 | 1,2,4 Oxadiazoles | >32 |
| 201 | 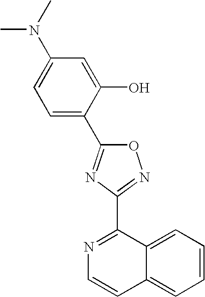 | 1,2,4 Oxadiazoles | >32 |
| 202 | 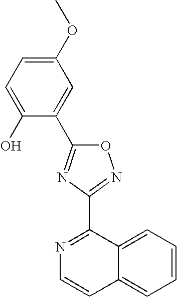 | 1,2,4 Oxadiazoles | 1.9 |
| 203 | 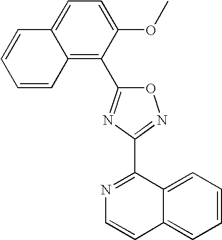 | 1,2,4 Oxadiazoles | 23 |
| 204 | 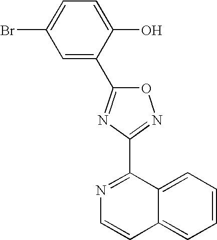 | 1,2,4 Oxadiazoles | 1.6 |
| 205 | 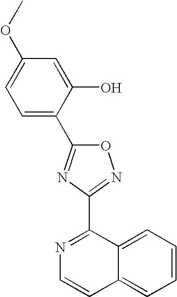 | 1,2,4 Oxadiazoles | 1 |
| 206 | 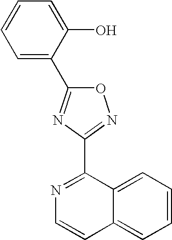 | 1,2,4 Oxadiazoles | 1.4 |
| 207 | 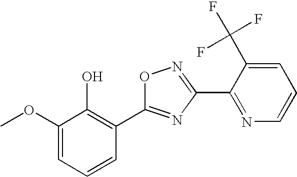 | 1,2,4 Oxadiazoles | 17 |
| 208 | 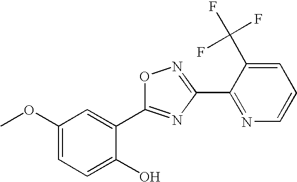 | 1,2,4 Oxadiazoles | 3.5 |
| 209 | 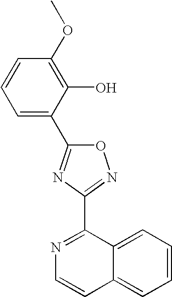 | 1,2,4 Oxadiazoles | 7.4 |
| 210 | 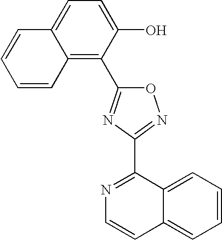 | 1,2,4 Oxadiazoles | 2.4 |
| 211 | 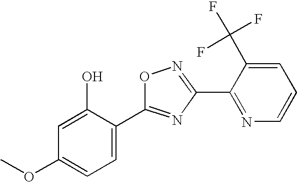 | 1,2,4 Oxadiazoles | 11 |
| 212 | 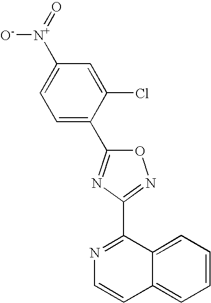 | 1,2,4 Oxadiazoles | >32 |
| 213 | 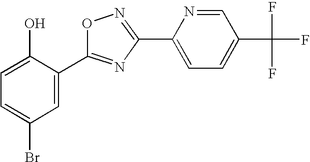 | 1,2,4 Oxadiazoles | 25 |
| 214 | 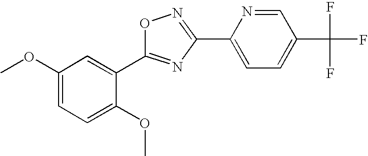 | 1,2,4 Oxadiazoles | 18 |
| 215 | 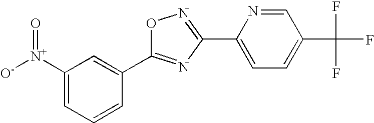 | 1,2,4 Oxadiazoles | 31 |
| 216 | 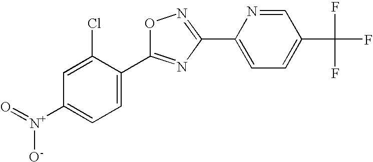 | 1,2,4 Oxadiazoles | 4.7 |
| 217 | 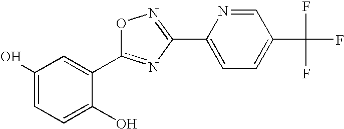 | 1,2,4 Oxadiazoles | 2.1 |
| 218 | 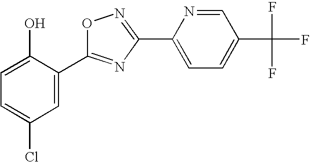 | 1,2,4 Oxadiazoles | 18 |
| 219 | 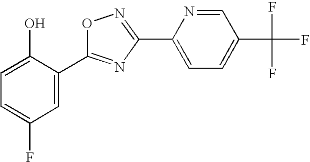 | 1,2,4 Oxadiazoles | >32 |
| 220 | 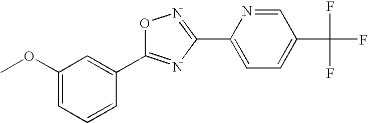 | 1,2,4 Oxadiazoles | >32 |
| 221 | 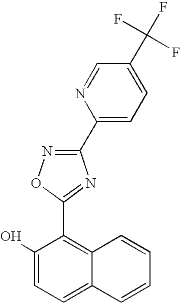 | 1,2,4 Oxadiazoles | 4.8 |
| 222 | 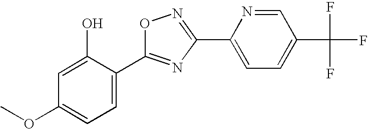 | 1,2,4 Oxadiazoles | >32 |
| 223 | 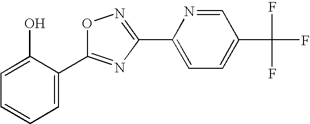 | 1,2,4 Oxadiazoles | 19 |
| 224 | 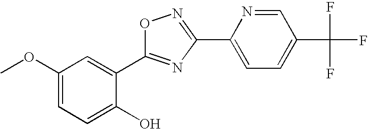 | 1,2,4 Oxadiazoles | 3.2 |
| 225 | 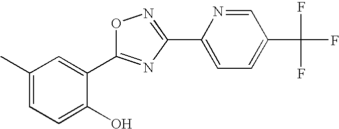 | 1,2,4 Oxadiazoles | 7.5 |
| 226 | 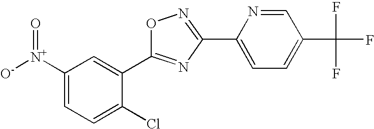 | 1,2,4 Oxadiazoles | 4.3 |
| 227 | 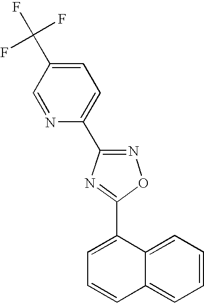 | 1,2,4 Oxadiazoles | >32 |
| 228 | 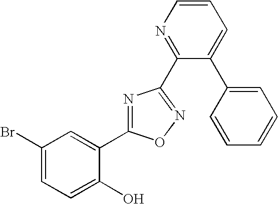 | 1,2,4 Oxadiazoles | 2.2 |
| 229 | 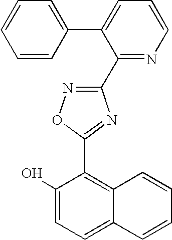 | 1,2,4 Oxadiazoles | >10 |
| 230 | 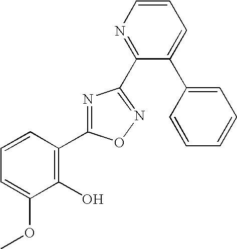 | 1,2,4 Oxadiazoles | 9.8 |
| 231 | 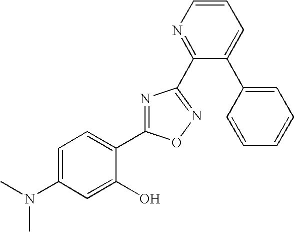 | 1,2,4 Oxadiazoles | 2.4 |
| 232 | 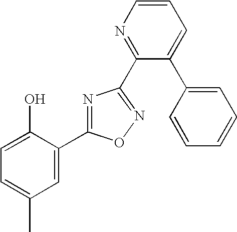 | 1,2,4 Oxadiazoles | >32 |
| 233 | 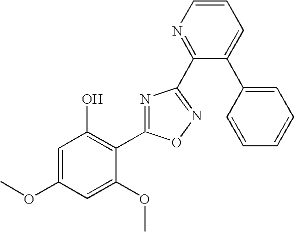 | 1,2,4 Oxadiazoles | 2.1 |
| 234 | 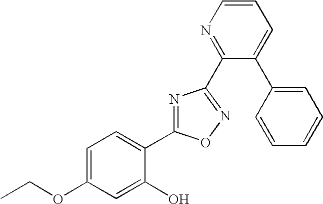 | 1,2,4 Oxadiazoles | >19 |
| 235 | 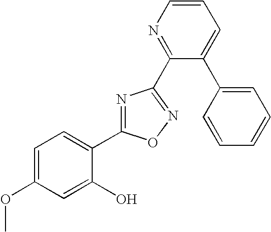 | 1,2,4 Oxadiazoles | 2.3 |
| 236 | 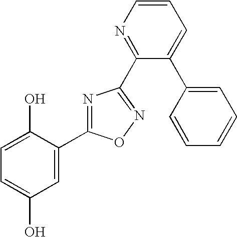 | 1,2,4 Oxadiazoles | 2.2 |
| 237 | 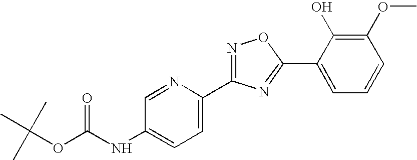 | 1,2,4 Oxadiazoles | >32 |
| 238 | 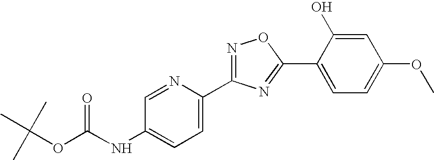 | 1,2,4 Oxadiazoles | 1.9 |
| 239 | 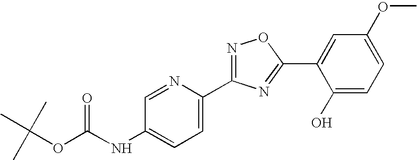 | 1,2,4 Oxadiazoles | 2.1 |
| 240 | 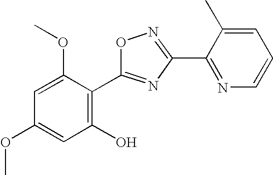 | 1,2,4 Oxadiazoles | 0.7 |
| 241 | 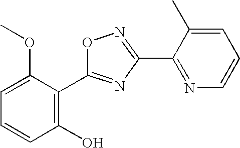 | 1,2,4 Oxadiazoles | >10 |
| 242 | 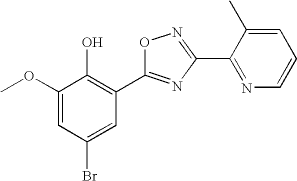 | 1,2,4 Oxadiazoles | 8.3 |
| 243 | 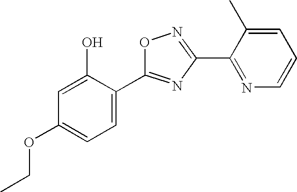 | 1,2,4 Oxadiazoles | 2.2 |
| 244 | 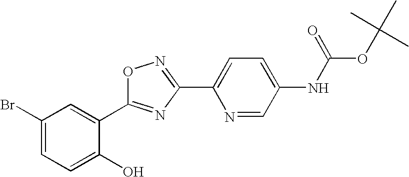 | 1,2,4 Oxadiazoles | 7.3 |
| 245 | 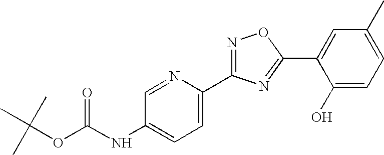 | 1,2,4 Oxadiazoles | >32 |
| 246 | 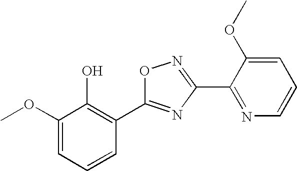 | 1,2,4 Oxadiazoles | 19 |
| 247 | 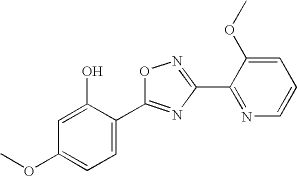 | 1,2,4 Oxadiazoles | 4.1 |
| 248 | 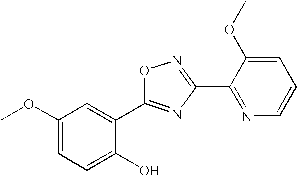 | 1,2,4 Oxadiazoles | 13 |
| 249 | 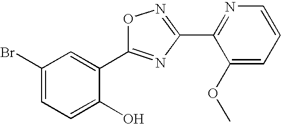 | 1,2,4 Oxadiazoles | 0.76 |
| 250 | 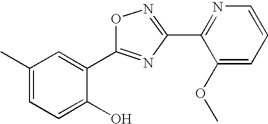 | 1,2,4 Oxadiazoles | 2 |
| 201 | 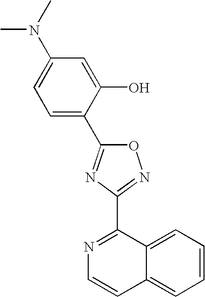 | 1,2,4 Oxadiazoles | >32 |
| 202 | 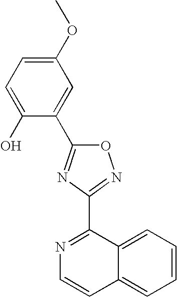 | 1,2,4 Oxadiazoles | 1.9 |
| 203 | 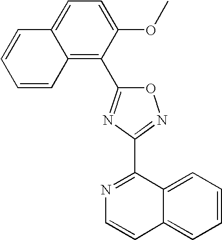 | 1,2,4 Oxadiazoles | 23 |
| 204 | 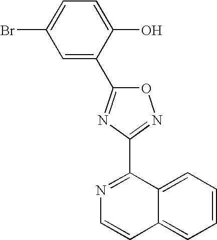 | 1,2,4 Oxadiazoles | 1.6 |
| 205 | 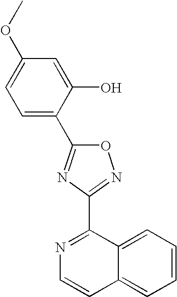 | 1,2,4 Oxadiazoles | 1 |
| 206 | 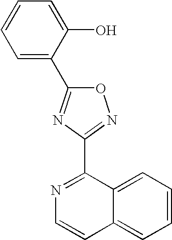 | 1,2,4 Oxadiazoles | 1.4 |
| 207 | 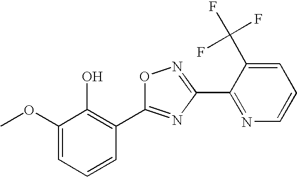 | 1,2,4 Oxadiazoles | 17 |
| 208 | 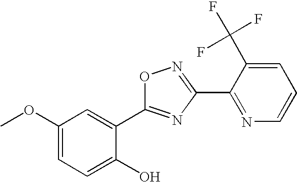 | 1,2,4 Oxadiazoles | 3.5 |
| 209 | 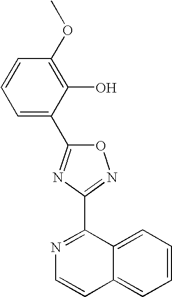 | 1,2,4 Oxadiazoles | 7.4 |
| 210 | 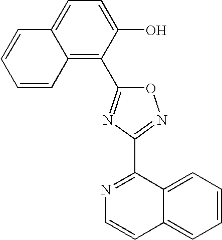 | 1,2,4 Oxadiazoles | 2.4 |

| | | | Mono |
|---|---|---|---|
| | | | EC50 |
| | | | Median |
| | Structure | Class | [uM] |
| 211 | 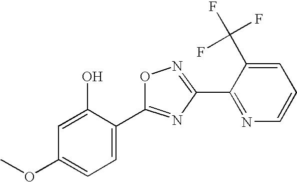 | 1,2,4 Oxadiazoles | 11 |
| 212 | 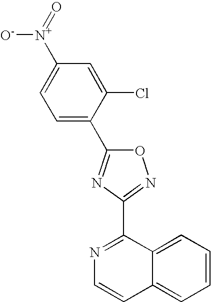 | 1,2,4 Oxadiazoles | >32 |
| 213 | 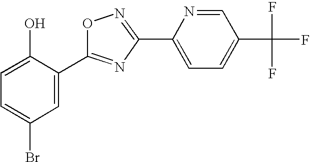 | 1,2,4 Oxadiazoles | 25 |
| 214 | 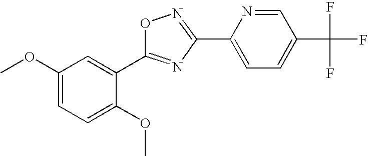 | 1,2,4 Oxadiazoles | 18 |
| 215 | 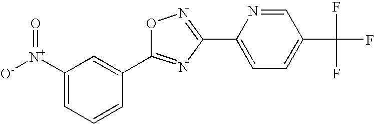 | 1,2,4 Oxadiazoles | 31 |
| 216 | 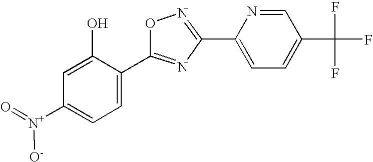 | 1,2,4 Oxadiazoles | 4.7 |
| 217 | 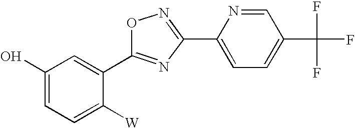 | 1,2,4 Oxadiazoles | 2.1 |
| 218 | 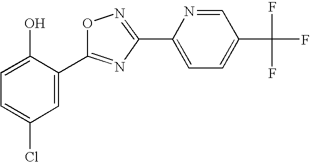 | 1,2,4 Oxadiazoles | 18 |
| 219 | 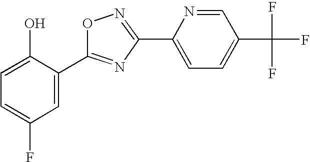 | 1,2,4 Oxadiazoles | >32 |
| 220 | 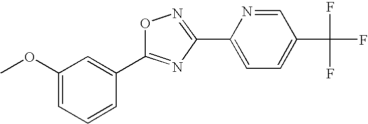 | 1,2,4 Oxadiazoles | >32 |
| 221 | 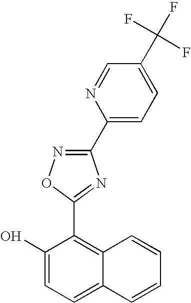 | 1,2,4 Oxadiazoles | 4.8 |
| 222 | 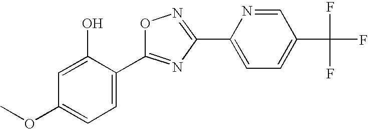 | 1,2,4 Oxadiazoles | >32 |
| 223 | 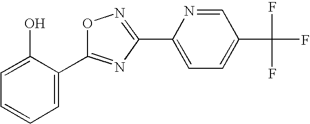 | 1,2,4 Oxadiazoles | 19 |
| 224 | 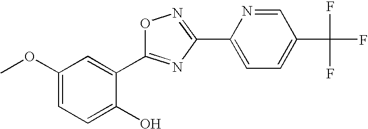 | 1,2,4 Oxadiazoles | 3.2 |
| 225 | 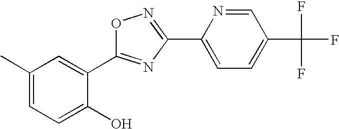 | 1,2,4 Oxadiazoles | 7.5 |
| 226 | 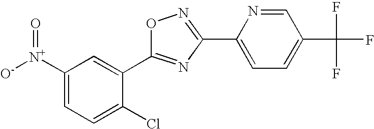 | 1,2,4 Oxadiazoles | 4.3 |
| 227 | 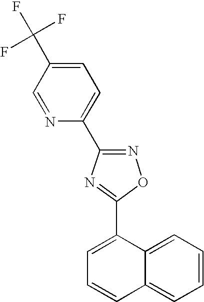 | 1,2,4 Oxadiazoles | >32 |
| 228 | 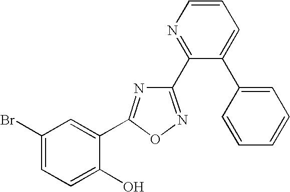 | 1,2,4 Oxadiazoles | 2.2 |
| 229 | 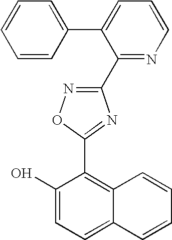 | 1,2,4 Oxadiazoles | >10 |
| 230 | 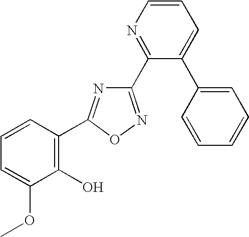 | 1,2,4 Oxadiazoles | 9.8 |
| 231 | 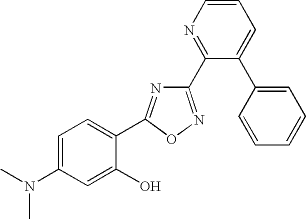 | 1,2,4 Oxadiazoles | 2.4 |
| 232 | 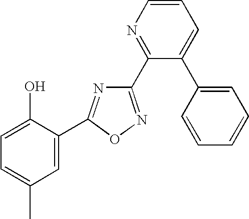 | 1,2,4 Oxadiazoles | >32 |
| 233 | 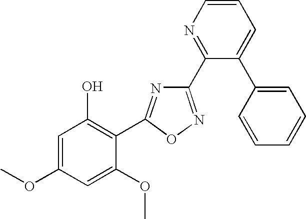 | 1,2,4 Oxadiazoles | 2.1 |
| 234 | 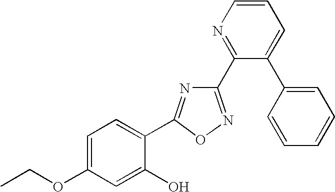 | 1,2,4 Oxadiazoles | >19 |
| 235 | 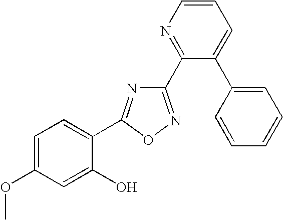 | 1,2,4 Oxadiazoles | 2.3 |
| 236 | 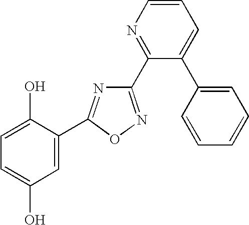 | 1,2,4 Oxadiazoles | 2.2 |
| 237 | 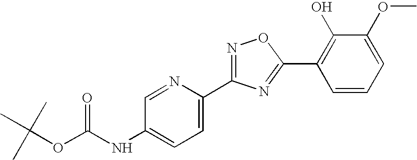 | 1,2,4 Oxadiazoles | >32 |
| 238 | 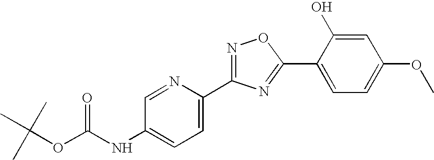 | 1,2,4 Oxadiazoles | 1.9 |
| 239 | 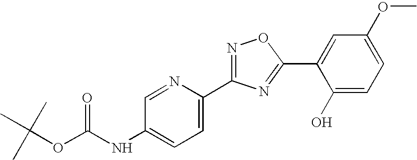 | 1,2,4 Oxadiazoles | 2.1 |
| 240 | 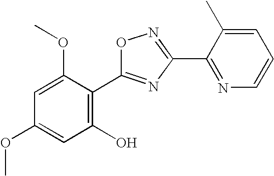 | 1,2,4 Oxadiazoles | 0.7 |
| 241 | 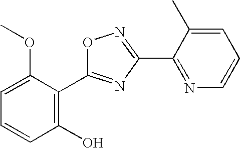 | 1,2,4 Oxadiazoles | >10 |
| 242 | 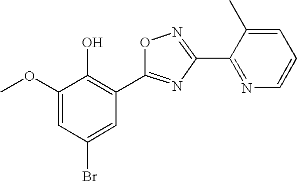 | 1,2,4 Oxadiazoles | 8.3 |
| 243 | 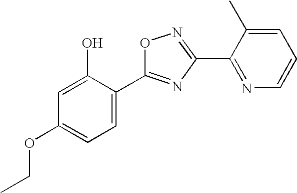 | 1,2,4 Oxadiazoles | 2.2 |
| 244 | 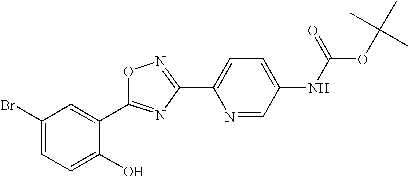 | 1,2,4 Oxadiazoles | 7.3 |
| 245 | 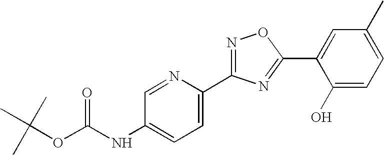 | 1,2,4 Oxadiazoles | >32 |
| 246 | 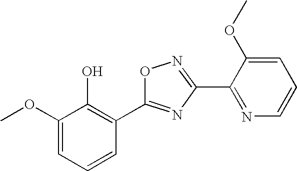 | 1,2,4 Oxadiazoles | 19 |
| 247 | 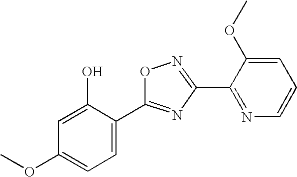 | 1,2,4 Oxadiazoles | 4.1 |
| 248 | 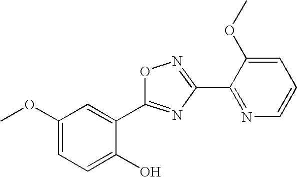 | 1,2,4 Oxadiazoles | 13 |
| 249 | 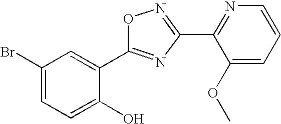 | 1,2,4 Oxadiazoles | 0.76 |
| 250 | 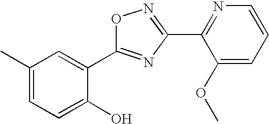 | 1,2,4 Oxadiazoles | 2 |
| 251 | 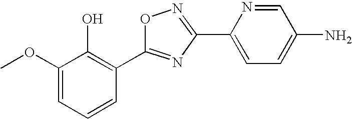 | 1,2,4 Oxadiazoles | >32 |
| 252 | 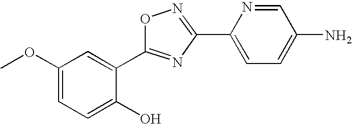 | 1,2,4 Oxadiazoles | 9.2 |
| 253 | 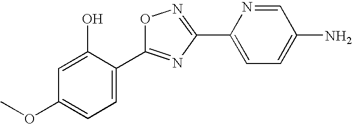 | 1,2,4 Oxadiazoles | >32 |
| 254 | 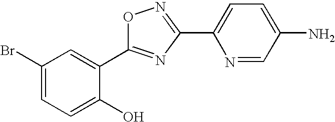 | 1,2,4 Oxadiazoles | 32 |
| 255 | 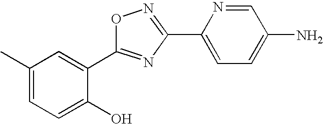 | 1,2,4 Oxadiazoles | 24 |
| 256 | 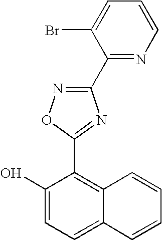 | 1,2,4 Oxadiazoles | 2.1 |
| 257 | 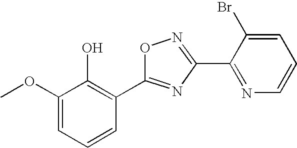 | 1,2,4 Oxadiazoles | >32 |
| 258 | 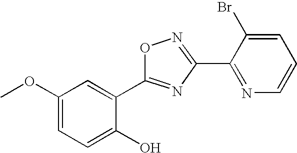 | 1,2,4 Oxadiazoles | >10 |
| 259 | 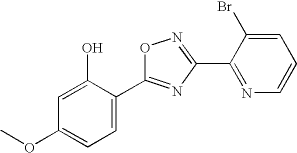 | 1,2,4 Oxadiazoles | 2.5 |
| 260 | 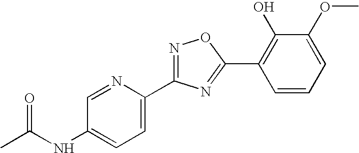 | 1,2,4 Oxadiazoles | 44.3 |
| 261 | 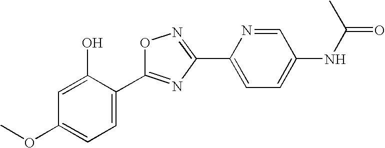 | 1,2,4 Oxadiazoles | 45.3 |
| 262 | 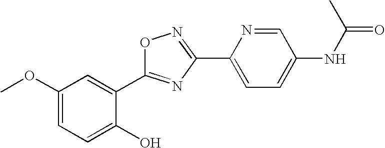 | 1,2,4 Oxadiazoles | 19.2 |
| 263 | 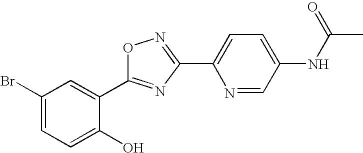 | 1,2,4 Oxadiazoles | >19 |
| 264 | 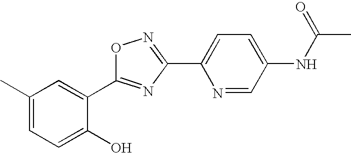 | 1,2,4 Oxadiazoles | >19 |
| 265 | 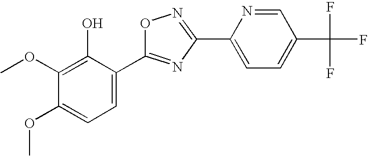 | 1,2,4 Oxadiazoles | 28.5 |
| 266 | 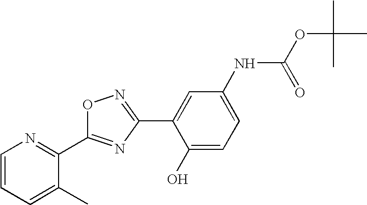 | 1,2,4 Oxadiazoles | 4.7 |
| 267 | 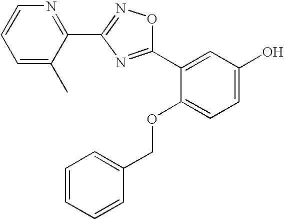 | 1,2,4 Oxadiazoles | 15.9 |
| 268 | 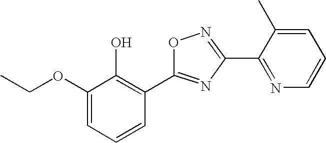 | 1,2,4 Oxadiazoles | 28.7 |
| 269 | 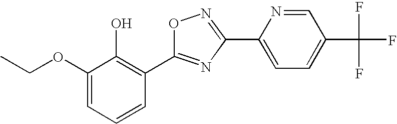 | 1,2,4 Oxadiazoles | 35.9 |
| 270 | 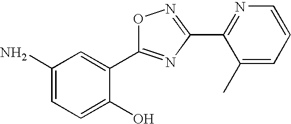 | 1,2,4 Oxadiazoles | 27.7 |
| 271 | 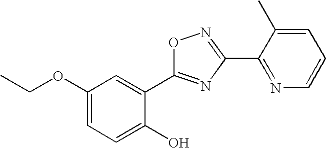 | 1,2,4 Oxadiazoles | 0.61 |
| 272 | 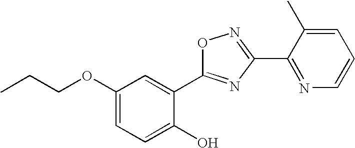 | 1,2,4 Oxadiazoles | >32 |
| 273 | 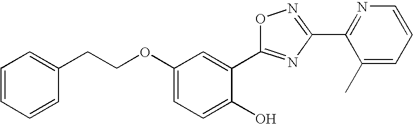 | 1,2,4 Oxadiazoles | >19 |
| 274 | 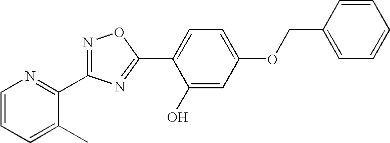 | 1,2,4 Oxadiazoles | 1.8 |
| 275 | 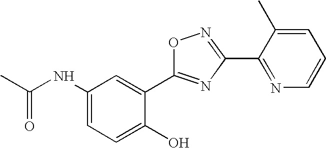 | 1,2,4 Oxadiazoles | 18 |
| 276 | 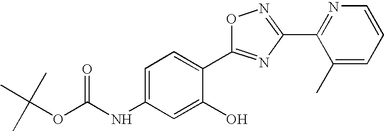 | 1,2,4 Oxadiazoles | 0.7 |
| 277 | 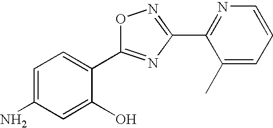 | 1,2,4 Oxadiazoles | 6 |
| 278 | 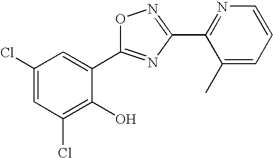 | 1,2,4 Oxadiazoles | 3.8 |
| 279 | 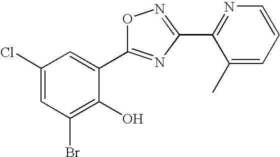 | 1,2,4 Oxadiazoles | 3.3 |
| 280 | 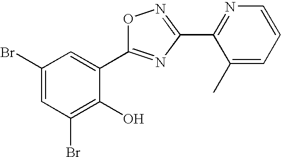 | 1,2,4 Oxadiazoles | 7.4 |
| 281 | 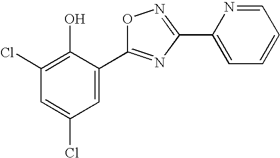 | 1,2,4 Oxadiazoles | 2.1 |
| 282 | 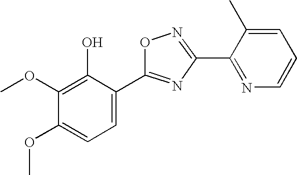 | 1,2,4 Oxadiazoles | 1.3 |
| 283 | 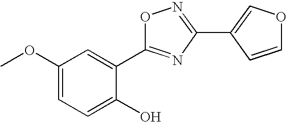 | 1,2,4 Oxadiazoles | 15 |
| 284 | 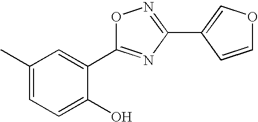 | 1,2,4 Oxadiazoles | 17 |
| 285 | 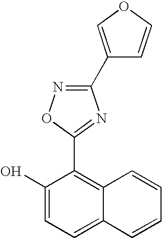 | 1,2,4 Oxadiazoles | 11 |
| 286 | 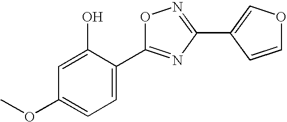 | 1,2,4 Oxadiazoles | 9.7 |
| 287 | 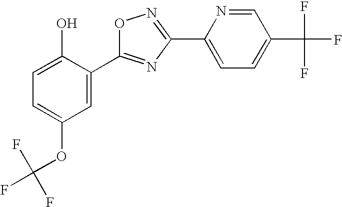 | 1,2,4 Oxadiazoles | 1.7 |
| 288 | 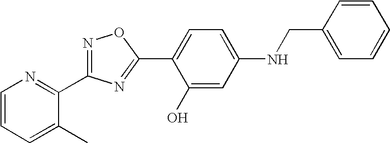 | 1,2,4 Oxadiazoles | 0.8 |
| 289 | 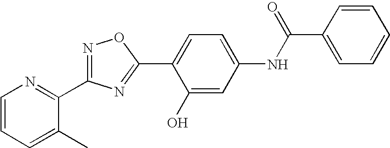 | 1,2,4 Oxadiazoles | 6.1 |
| 290 | 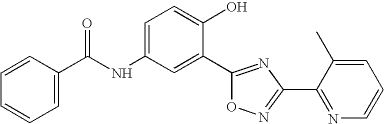 | 1,2,4 Oxadiazoles | 2.8 |
| 291 | 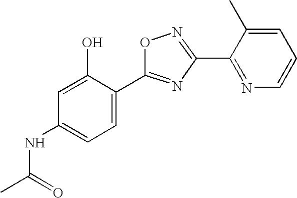 | 1,2,4 Oxadiazoles | 6 |
| 292 | 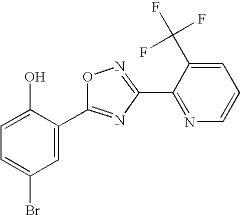 | 1,2,4 Oxadiazoles | 1.5 |
| 293 | 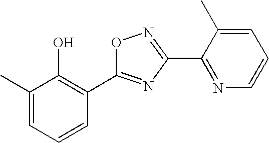 | 1,2,4 Oxadiazoles | 1.7 |
| 294 | 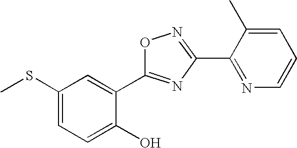 | 1,2,4 Oxadiazoles | 0.2 |
| 295 | 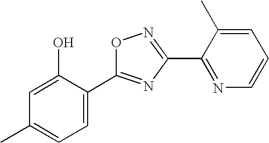 | 1,2,4 Oxadiazoles | 2.1 |
| 296 | 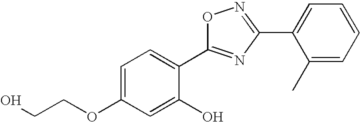 | 1,2,4 Oxadiazoles | 2.5 |
| 297 | 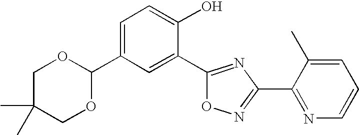 | 1,2,4 Oxadiazoles | 0.43 |
| 298 | 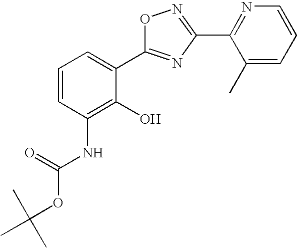 | 1,2,4 Oxadiazoles | 0.62 |
| 299 | 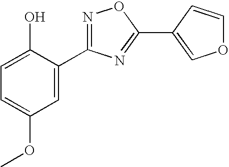 | 1,2,4 Oxadiazoles | 21.7 |
| 300 | 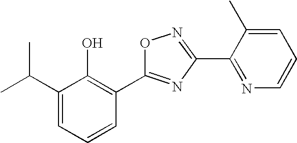 | 1,2,4 Oxadiazoles | 1.5 |
| 301 | 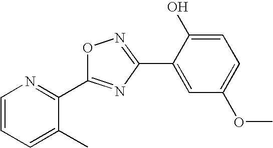 | 1,2,4 Oxadiazoles | >32 |
| 302 | 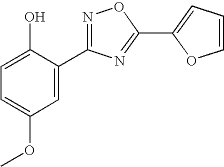 | 1,2,4 Oxadiazoles | 16 |
| 303 | 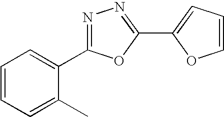 | 1,3,4 Oxadiazoles | >32 |
| 304 | 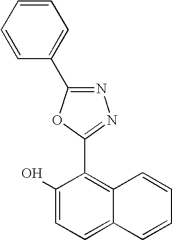 | 1,3,4 Oxadiazoles | >32 |
| 305 | 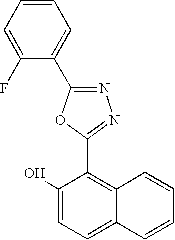 | 1,3,4 Oxadiazoles | >32 |
| 306 | 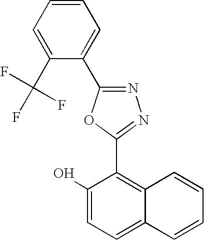 | 1,3,4 Oxadiazoles | 26 |
| 307 | 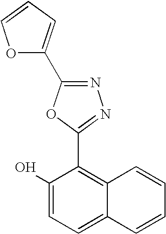 | 1,3,4 Oxadiazoles | >32 |
| 308 | 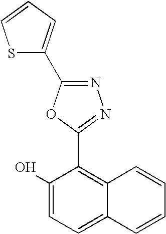 | 1,3,4 Oxadiazoles | 26 |
| 309 | 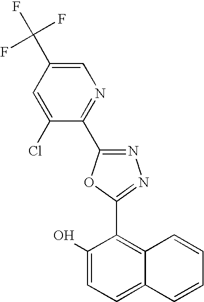 | 1,3,4 Oxadiazoles | 16 |
| 310 | 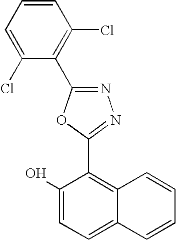 | 1,3,4 Oxadiazoles | 4.4 |
| 311 | 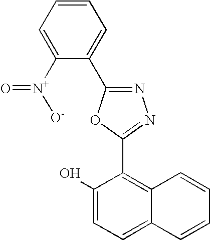 | 1,3,4 Oxadiazoles | 4 |
| 312 | 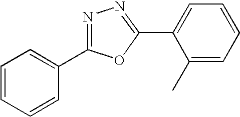 | 1,3,4 Oxadiazoles | >32 |
| 313 | 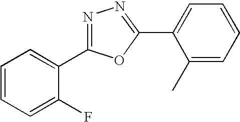 | 1,3,4 Oxadiazoles | >32 |
| 314 | 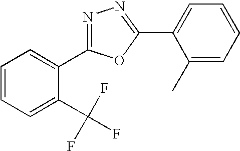 | 1,3,4 Oxadiazoles | 20 |
| 315 | 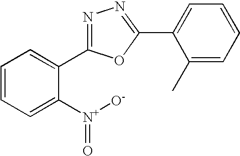 | 1,3,4 Oxadiazoles | >32 |
| 316 | 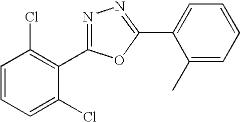 | 1,3,4 Oxadiazoles | >32 |
| 317 | 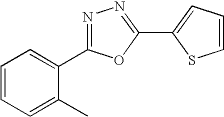 | 1,3,4 Oxadiazoles | >32 |
| 318 | 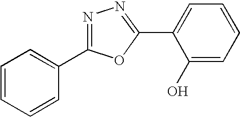 | 1,3,4 Oxadiazoles | 14 |
| 319 | 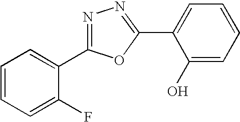 | 1,3,4 Oxadiazoles | 18 |
| 320 | 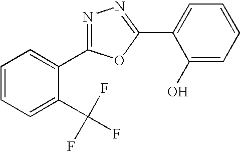 | 1,3,4 Oxadiazoles | >32 |
| 321 | 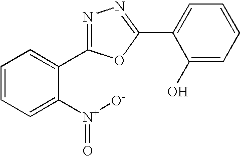 | 1,3,4 Oxadiazoles | >32 |
| 322 | 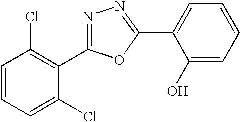 | 1,3,4 Oxadiazoles | 12 |
| 323 | 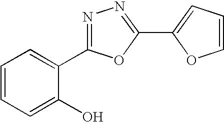 | 1,3,4 Oxadiazoles | >32 |
| 324 | 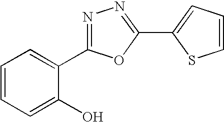 | 1,3,4 Oxadiazoles | >32 |
| 325 | 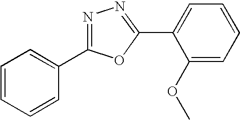 | 1,3,4 Oxadiazoles | >32 |
| 326 | 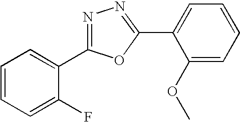 | 1,3,4 Oxadiazoles | >32 |
| 327 | 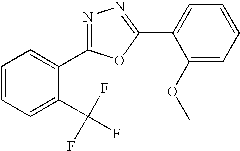 | 1,3,4 Oxadiazoles | 25 |
| 328 | 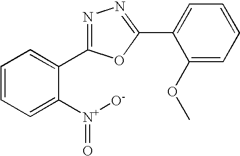 | 1,3,4 Oxadiazoles | >32 |
| 329 | 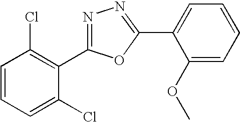 | 1,3,4 Oxadiazoles | >32 |
| 330 | 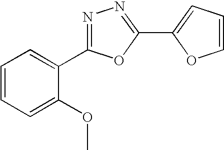 | 1,3,4 Oxadiazoles | >32 |
| 331 | 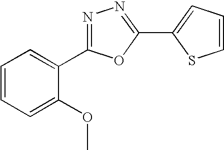 | 1,3,4 Oxadiazoles | >32 |
| 332 | 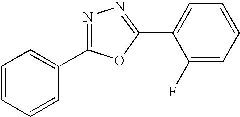 | 1,3,4 Oxadiazoles | >32 |
| 333 | 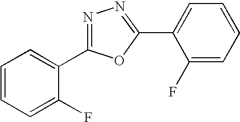 | 1,3,4 Oxadiazoles | >32 |
| 334 | 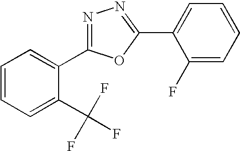 | 1,3,4 Oxadiazoles | >32 |
| 335 | 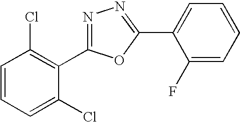 | 1,3,4 Oxadiazoles | >32 |
| 336 | 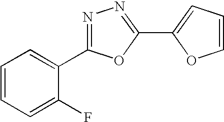 | 1,3,4 Oxadiazoles | >32 |
| 337 | 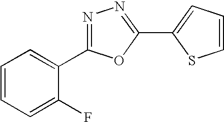 | 1,3,4 Oxadiazoles | >32 |
| 338 | 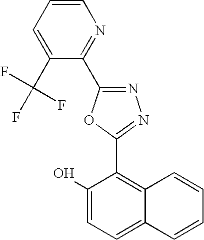 | 1,3,4 Oxadiazoles | >19 |
| 339 | 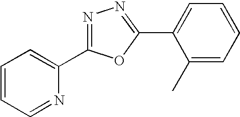 | 1,3,4 Oxadiazoles | >32 |
| 340 | 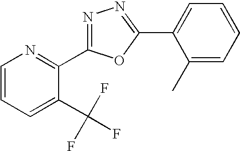 | 1,3,4 Oxadiazoles | >32 |
| 341 | 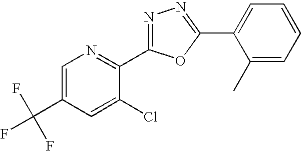 | 1,3,4 Oxadiazoles | 20 |
| 342 | 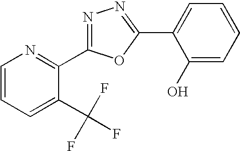 | 1,3,4 Oxadiazoles | >32 |
| 343 | 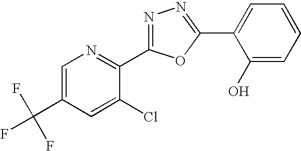 | 1,3,4 Oxadiazoles | 17 |
| 344 | 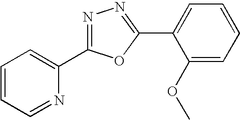 | 1,3,4 Oxadiazoles | >32 |
| 345 | 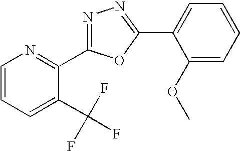 | 1,3,4 Oxadiazoles | 8.3 |
| 346 | 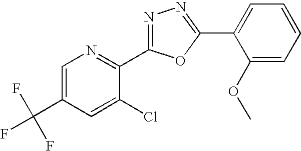 | 1,3,4 Oxadiazoles | >32 |
| 347 | 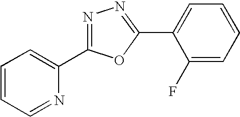 | 1,3,4 Oxadiazoles | >32 |
| 348 | 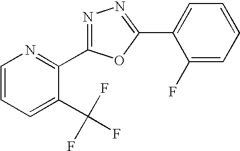 | 1,3,4 Oxadiazoles | >32 |
| 349 | 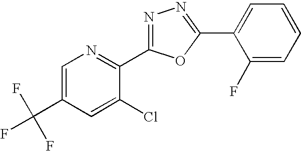 | 1,3,4 Oxadiazoles | >32 |
| 350 | 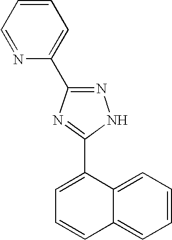 | Triazoles | >19 |
| 351 | 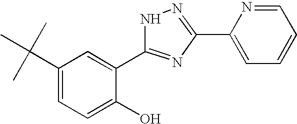 | Triazoles | 8.6 |
| 352 | 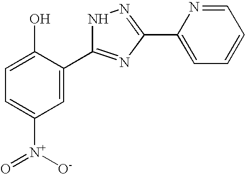 | Triazoles | 14 |
| 353 | 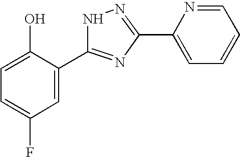 | Triazoles | 7.1 |
| 354 | 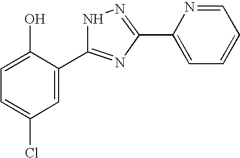 | Triazoles | 4.8 |
| 355 | 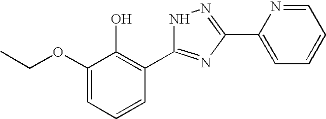 | Triazoles | >19 |
| 356 | 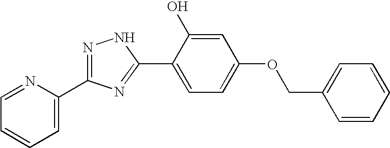 | Triazoles | >19 |
| 357 | 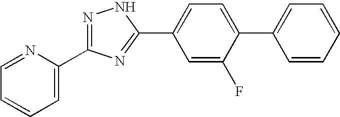 | Triazoles | 7.1 |
| 358 | 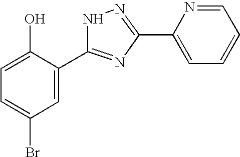 | Triazoles | 4.3 |
| 359 | 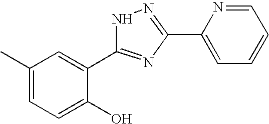 | Triazoles | >19 |
| 360 | 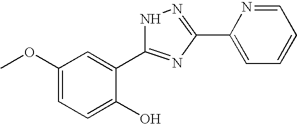 | Triazoles | 5.4 |
| 361 | 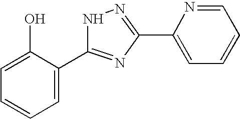 | Triazoles | >19 |
| 362 | 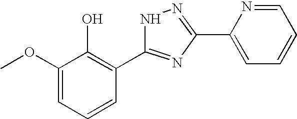 | Triazoles | >19 |
| 363 | 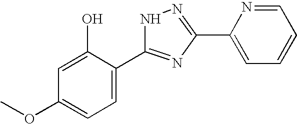 | Triazoles | >19 |
| 364 | 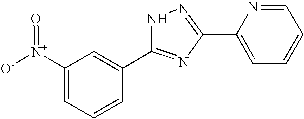 | Triazoles | >32 |
| 365 | 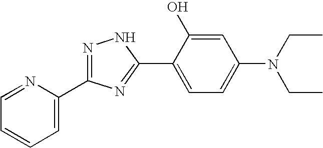 | Triazoles | >32 |
| 366 | 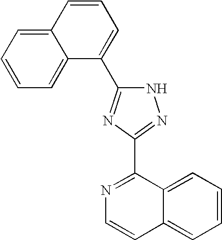 | Triazoles | >32 |
| 367 | 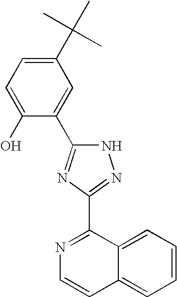 | Triazoles | >32 |
| 368 | 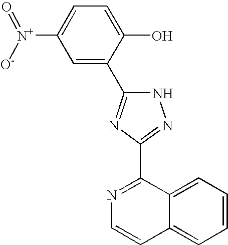 | Triazoles | >32 |
| 369 | 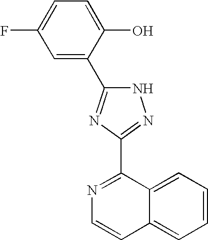 | Triazoles | 3.5 |
| 370 | 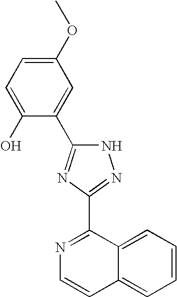 | Triazoles | 2.9 |
| 371 | 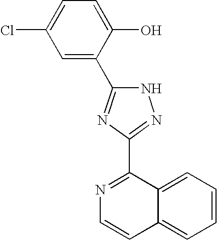 | Triazoles | >19 |
| 372 | 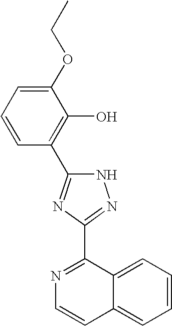 | Triazoles | >19 |
| 373 | 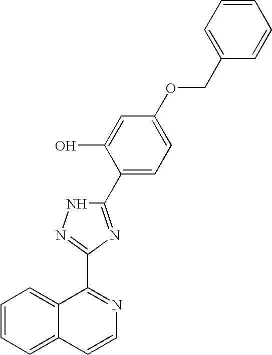 | Triazoles | >19 |
| 374 | 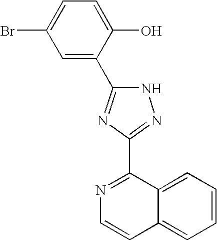 | Triazoles | >19 |
| 375 | 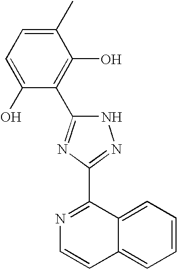 | Triazoles | >19 |
| 376 | 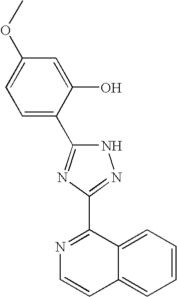 | Triazoles | >19 |
| 377 | 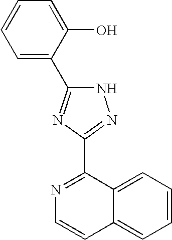 | Triazoles | >19 |
| 378 | 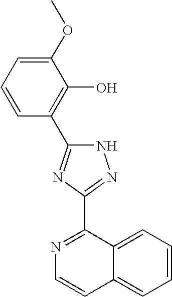 | Triazoles | 16 |
| 379 | 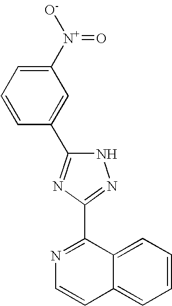 | Triazoles | >19 |
| 380 | 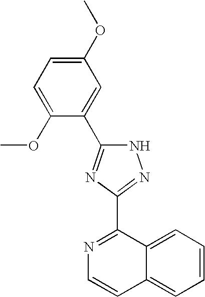 | Triazoles | >19 |
| 381 | 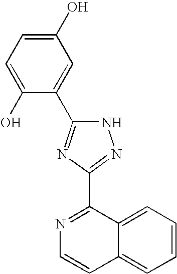 | Triazoles | 8.5 |
| 382 | 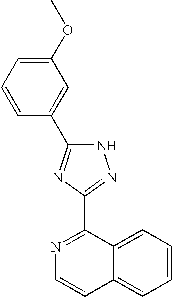 | Triazoles | >19 |
| 383 | 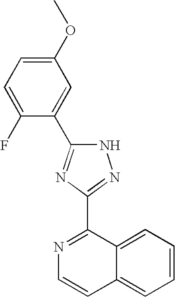 | Triazoles | >19 |
| 384 | 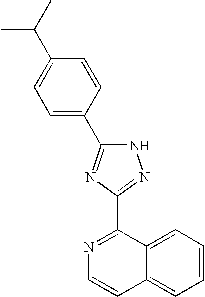 | Triazoles | 12 |
| 385 | 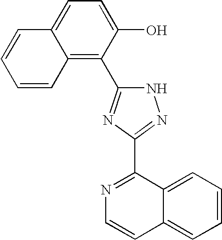 | Triazoles | >19 |
| 386 | 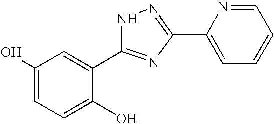 | Triazoles | >19 |
| 387 | 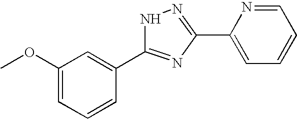 | Triazoles | >19 |
| 388 | 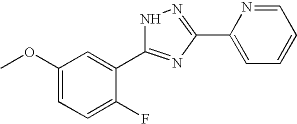 | Triazoles | >19 |
| 389 | 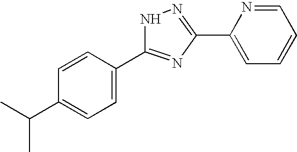 | Triazoles | >19 |
| 390 | 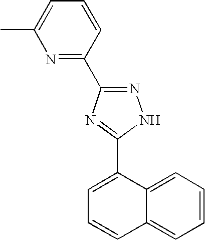 | Triazoles | >19 |
| 391 | 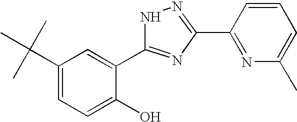 | Triazoles | >19 |
| 392 | 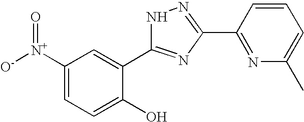 | Triazoles | >19 |
| 393 | 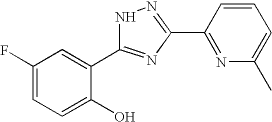 | Triazoles | >19 |
| 394 | 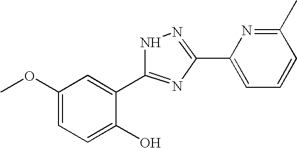 | Triazoles | >19 |
| 395 | 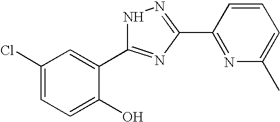 | Triazoles | >19 |
| 396 | 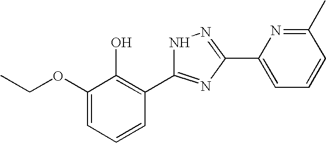 | Triazoles | >19 |
| 397 | 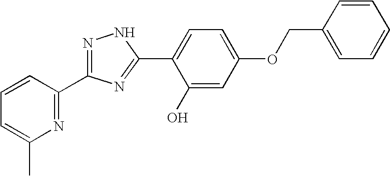 | Triazoles | >19 |
| 398 | 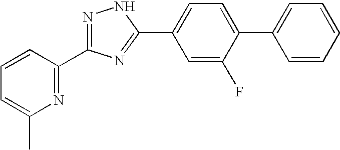 | Triazoles | >19 |
| 399 | 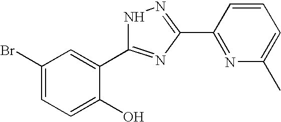 | Triazoles | >19 |
| 400 | 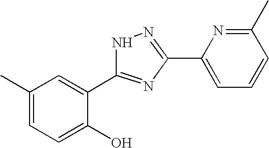 | Triazoles | >19 |
| 401 | 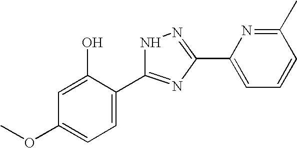 | Triazoles | >19 |
| 402 | 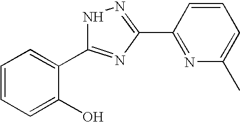 | Triazoles | >19 |
| 403 | 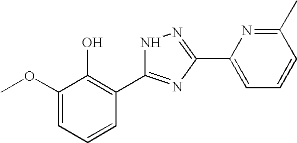 | Triazoles | >19 |
| 404 | 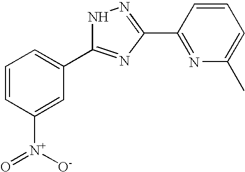 | Triazoles | >19 |
| 405 | 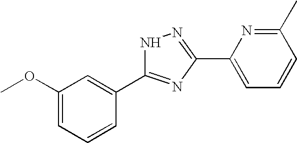 | Triazoles | >19 |
| 406 | 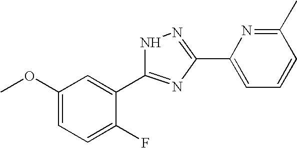 | Triazoles | >19 |
| 407 | 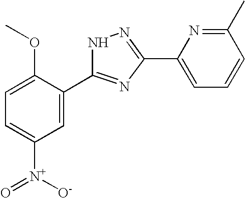 | Triazoles | >19 |
| 408 | 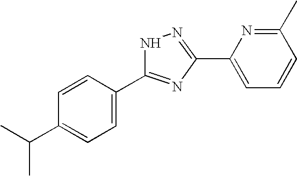 | Triazoles | >19 |
| 409 | 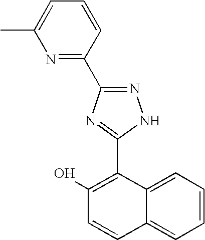 | Triazoles | >19 |
| 410 | 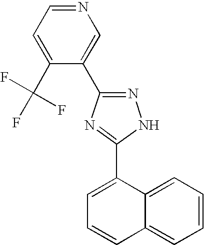 | Triazoles | 2.9 |
| 411 | 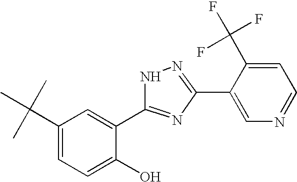 | Triazoles | 10 |
| 412 | 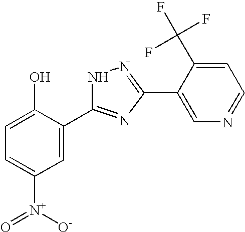 | Triazoles | >19 |
| 413 | 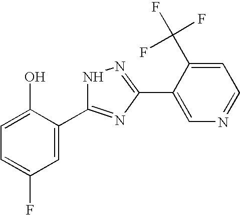 | Triazoles | >19 |
| 414 | 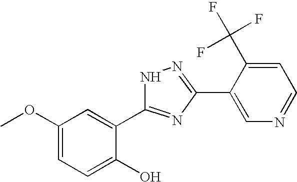 | Triazoles | >19 |
| 415 | 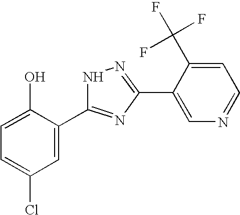 | Triazoles | 14 |
| 416 | 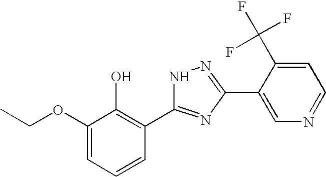 | Triazoles | >19 |
| 417 | 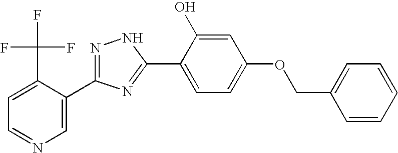 | Triazoles | >19 |
| 418 | 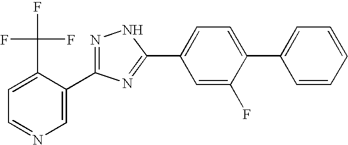 | Triazoles | 17 |
| 419 | 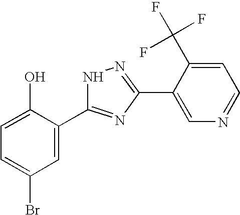 | Triazoles | 12 |
| 420 | 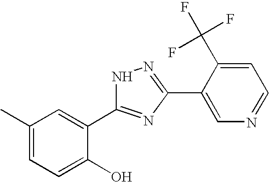 | Triazoles | 9.7 |
| 421 | 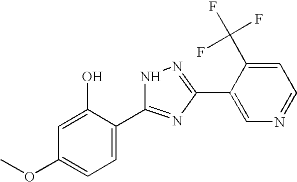 | Triazoles | >19 |
| 422 | 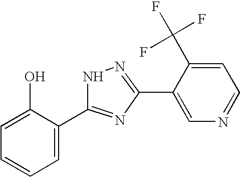 | Triazoles | >19 |
| 423 | 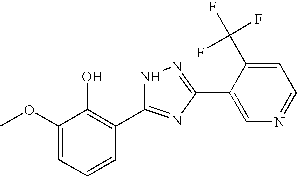 | Triazoles | >19 |
| 424 | 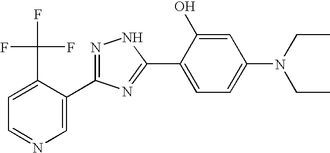 | Triazoles | >3.2 |
| 425 | 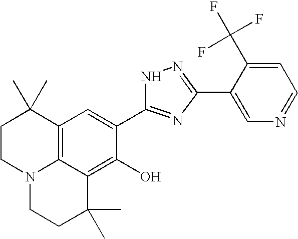 | Triazoles | 2.9 |
| 426 | 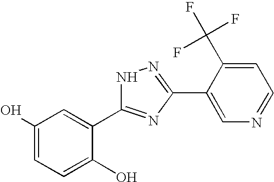 | Triazoles | 12 |
| 427 | 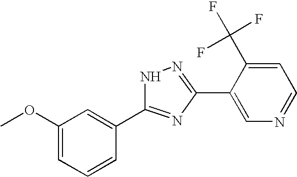 | Triazoles | >19 |
| 428 | 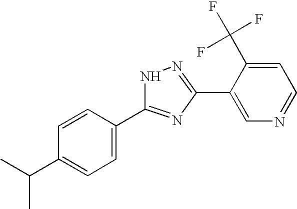 | Triazoles | >19 |
| 429 | 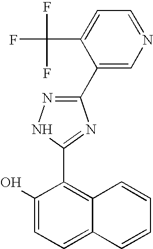 | Triazoles | >19 |
| 430 | 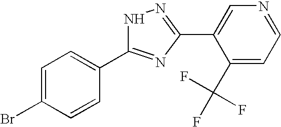 | Triazoles | >19 |
| 431 | 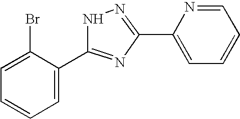 | Triazoles | >19 |
| 432 | 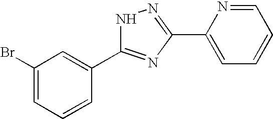 | Triazoles | 19 |
| 433 | 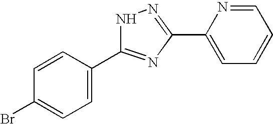 | Triazoles | >19 |
| 434 | 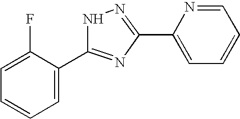 | Triazoles | >19 |
| 435 | 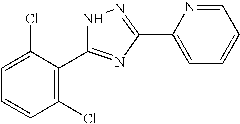 | Triazoles | >19 |
| 436 | 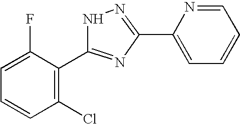 | Triazoles | 16 |
| 437 | 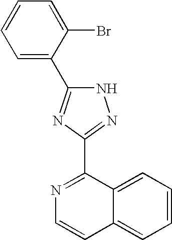 | Triazoles | 2.7 |
| 438 | 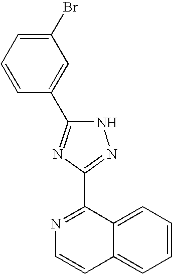 | Triazoles | >19 |
| 439 | 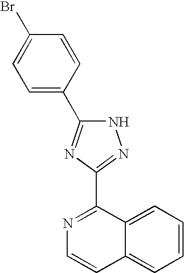 | Triazoles | >19 |
| 440 | 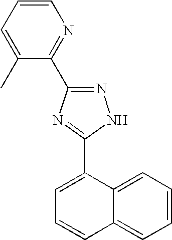 | Triazoles | 5.6 |
| 441 | 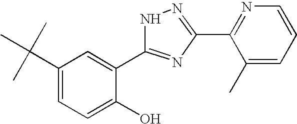 | Triazoles | >10 |
| 442 | 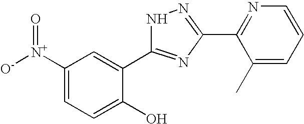 | Triazoles | 7.9 |
| 443 | 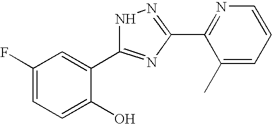 | Triazoles | >10 |
| 444 | 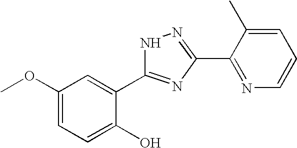 | Triazoles | 7.7 |
| 445 | 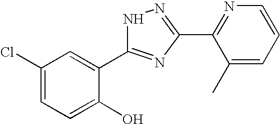 | Triazoles | 2.9 |
| 446 | 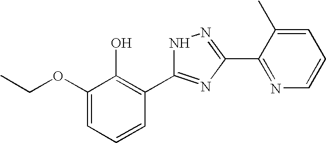 | Triazoles | 7.8 |
| 447 | 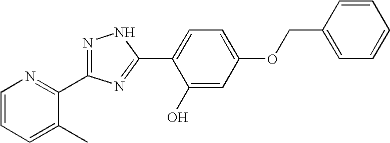 | Triazoles | >10 |
| 448 | 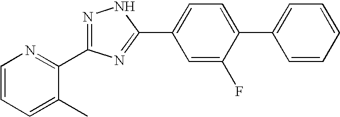 | Triazoles | 28 |
| 449 | 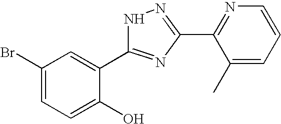 | Triazoles | 2.3 |
| 450 | 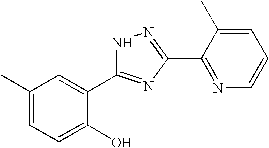 | Triazoles | 4.2 |
| 451 | 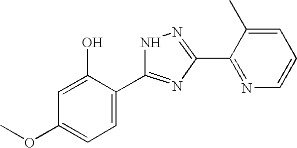 | Triazoles | 23 |
| 452 | 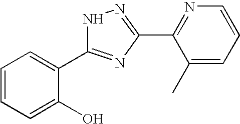 | Triazoles | 31 |
| 453 | 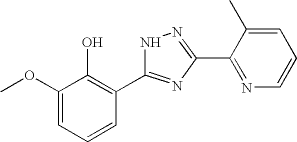 | Triazoles | 21 |
| 454 | 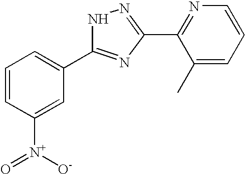 | Triazoles | 24 |
| 455 | 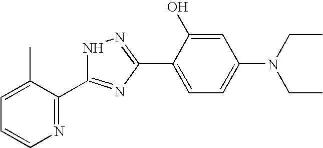 | Triazoles | 8.1 |
| 456 | 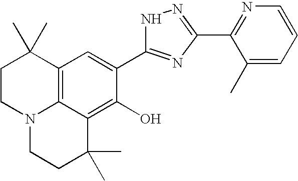 | Triazoles | 0.73 |
| 457 | 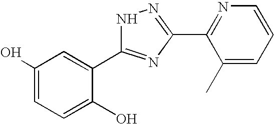 | Triazoles | 4.1 |
| 458 | 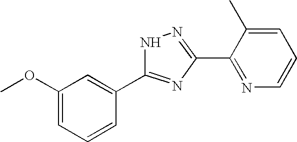 | Triazoles | >32 |
| 459 | 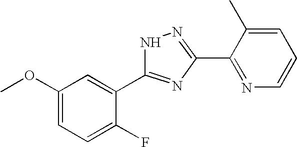 | Triazoles | >32 |
| 460 | 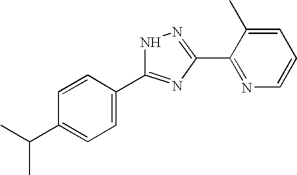 | Triazoles | 3 |
| 461 | 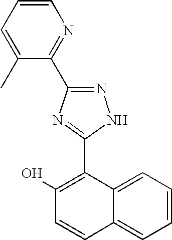 | Triazoles | 6.9 |
| 462 | 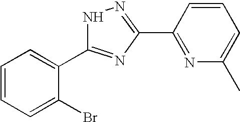 | Triazoles | >32 |
| 463 | 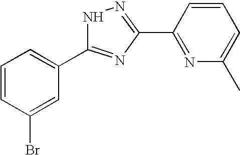 | Triazoles | 19 |
| 464 | 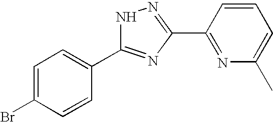 | Triazoles | >32 |
| 465 | 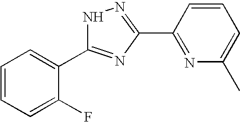 | Triazoles | >32 |
| 466 | 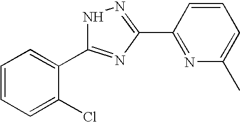 | Triazoles | >32 |
| 467 | 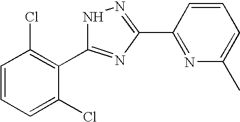 | Triazoles | >32 |
| 468 | 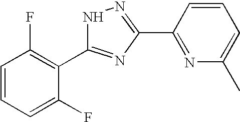 | Triazoles | >32 |
| 469 | 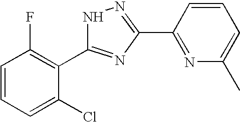 | Triazoles | >32 |
| 470 | 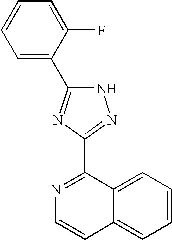 | Triazoles | 7.2 |

| | | | Mono EC50 |
|---|---|---|---|
| | Structure | Class | Median [uM] |
| 471 | 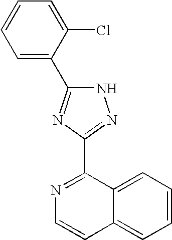 | Triazoles | 6.1 |
| 472 | 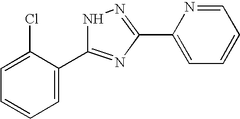 | Triazoles | >32 |
| 473 | 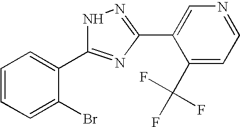 | Triazoles | >32 |
| 474 | 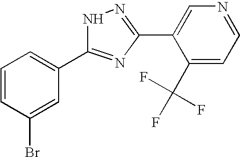 | Triazoles | >32 |
| 475 | 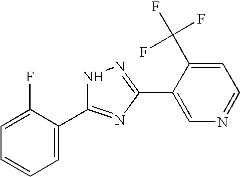 | Triazoles | >32 |
| 476 | 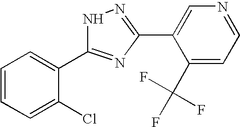 | Triazoles | >32 |
| 477 | 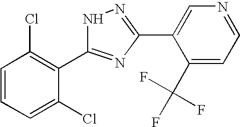 | Triazoles | >32 |
| 478 | 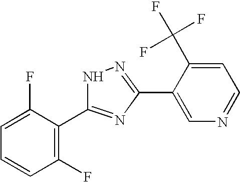 | Triazoles | >32 |
| 479 | 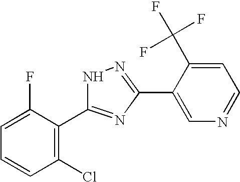 | Triazoles | >32 |
| 480 | 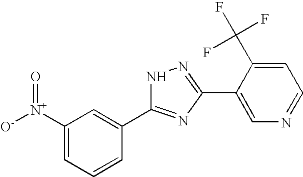 | Triazoles | >32 |
| 481 | 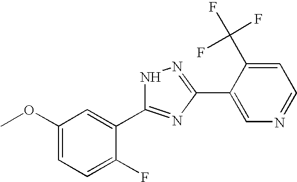 | Triazoles | >32 |
| 482 | 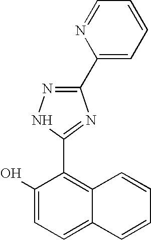 | Triazoles | >19 |
| 483 | 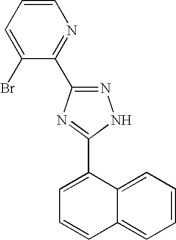 | Triazoles | >32 |
| 484 | 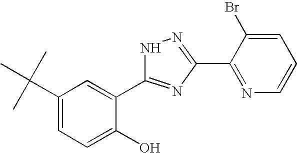 | Triazoles | 7.2 |
| 485 | 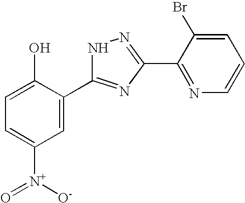 | Triazoles | >19 |
| 486 | 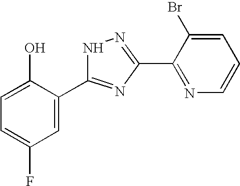 | Triazoles | 7.4 |
| 487 | 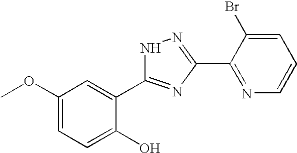 | Triazoles | >32 |
| 488 | 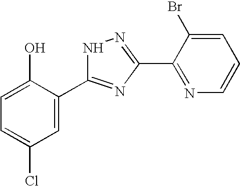 | Triazoles | 2.7 |
| 489 | 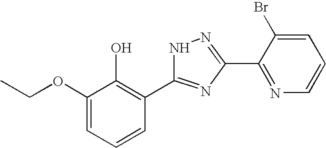 | Triazoles | 22 |
| 490 | 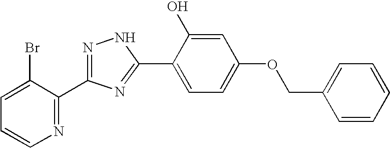 | Triazoles | 7 |
| 491 | 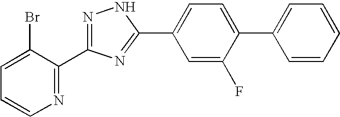 | Triazoles | 2.3 |
| 492 | 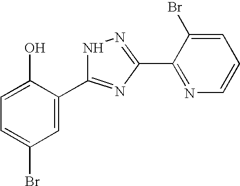 | Triazoles | 5.4 |
| 493 | 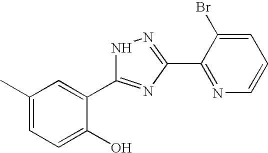 | Triazoles | 16 |
| 494 | 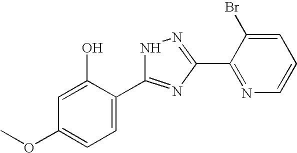 | Triazoles | >32 |
| 495 | 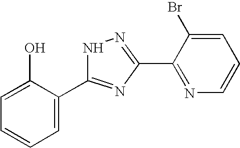 | Triazoles | 15 |
| 496 | 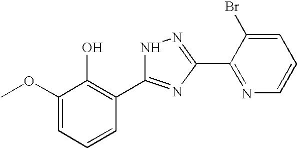 | Triazoles | >32 |
| 497 | 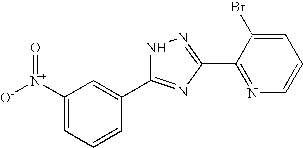 | Triazoles | >32 |
| 498 | 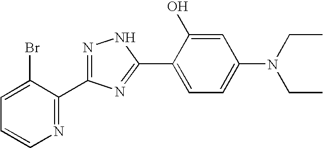 | Triazoles | 5.8 |
| 499 | 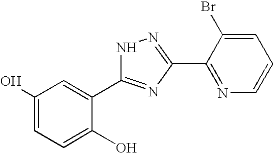 | Triazoles | 6.2 |
| 500 | 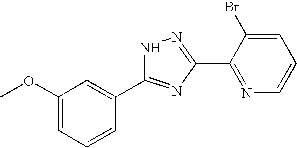 | Triazoles | >32 |
| 501 | 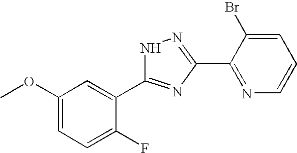 | Triazoles | >32 |
| 502 | 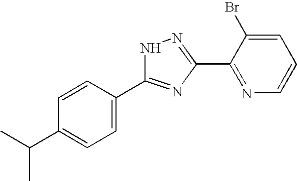 | Triazoles | 12 |
| 503 | 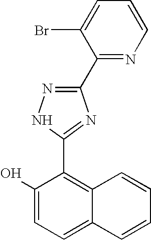 | Triazoles | 6.9 |
| 504 | 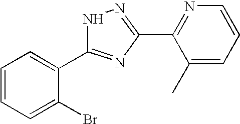 | Triazoles | 7.3 |
| 505 | 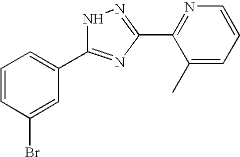 | Triazoles | 4 |
| 506 | 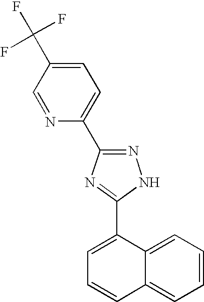 | Triazoles | >19 |
| 507 | 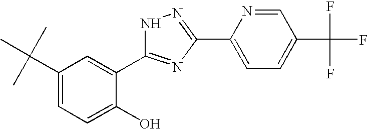 | Triazoles | >19 |
| 508 | 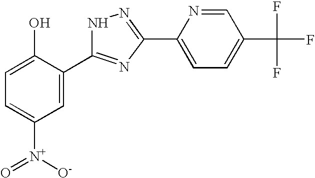 | Triazoles | >19 |
| 509 | 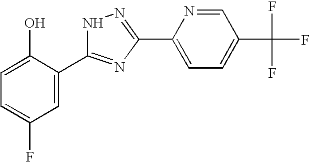 | Triazoles | >19 |
| 510 | 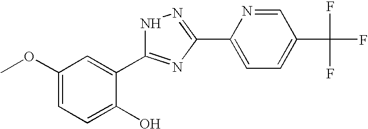 | Triazoles | >19 |
| 511 | 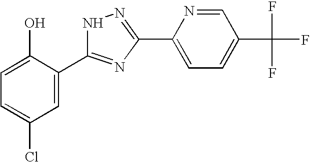 | Triazoles | >19 |
| 512 | 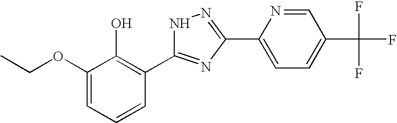 | Triazoles | >19 |
| 513 | 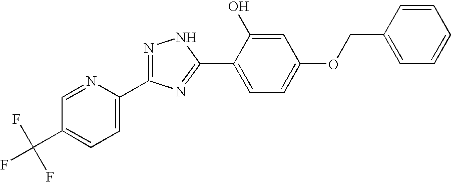 | Triazoles | >19 |
| 514 | 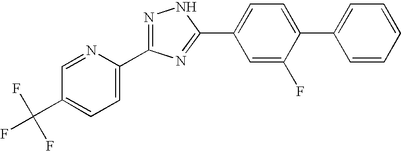 | Triazoles | >19 |
| 515 | 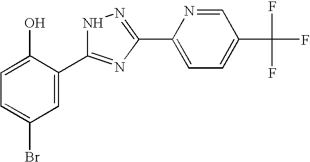 | Triazoles | >19 |
| 516 | 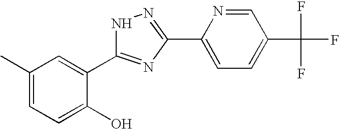 | Triazoles | >19 |
| 517 | 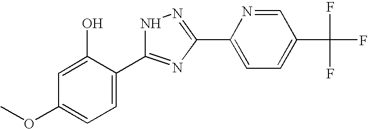 | Triazoles | >19 |
| 518 | 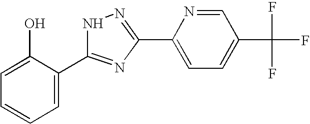 | Triazoles | >19 |
| 519 | 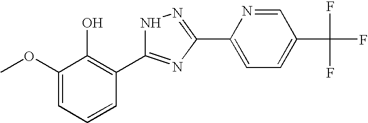 | Triazoles | >19 |
| 520 | 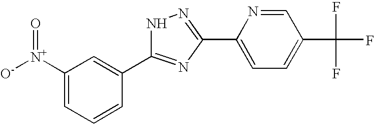 | Triazoles | >19 |
| 521 | 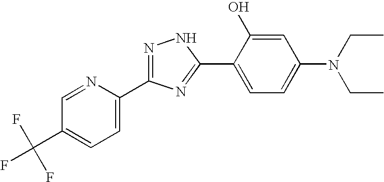 | Triazoles | >19 |
| 522 | 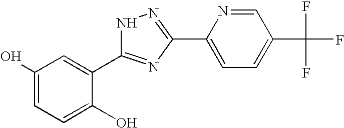 | Triazoles | >19 |
| 523 | 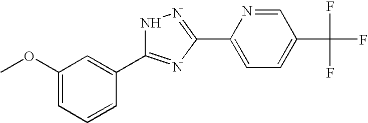 | Triazoles | >19 |
| 524 | 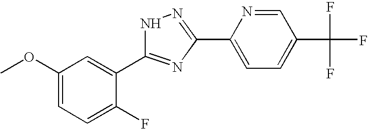 | Triazoles | >19 |
| 525 | 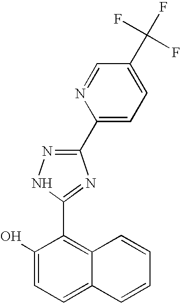 | Triazoles | >19 |
| 526 | 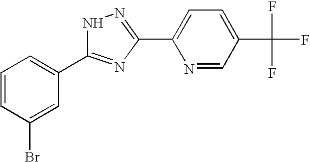 | Triazoles | >19 |
| 527 | 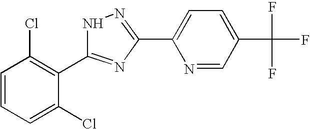 | Triazoles | >19 |
| 528 | 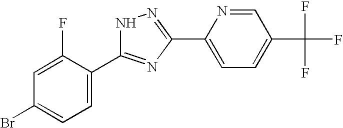 | Triazoles | >19 |
| 529 | 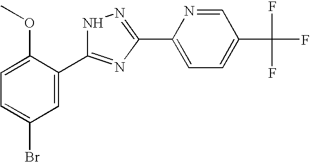 | Triazoles | >19 |
| 530 | 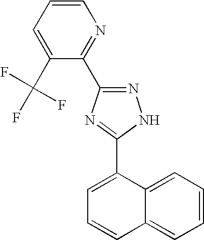 | Triazoles | >19 |
| 531 | 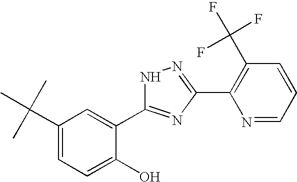 | Triazoles | >19 |
| 532 | 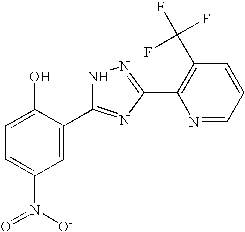 | Triazoles | >19 |
| 533 | 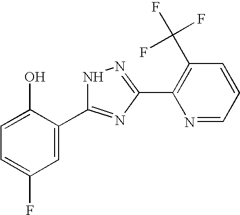 | Triazoles | >19 |
| 534 | 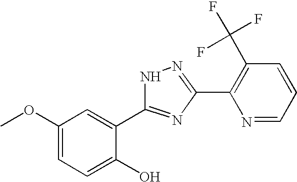 | Triazoles | 2.3 |
| 535 | 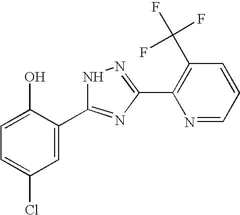 | Triazoles | 4.1 |
| 536 | 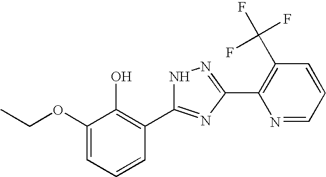 | Triazoles | >19 |
| 537 | 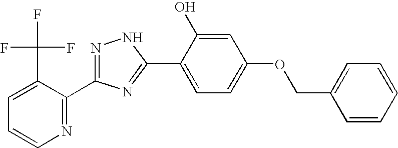 | Triazoles | >19 |
| 538 | 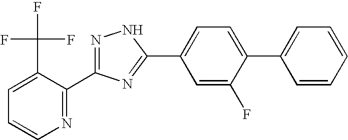 | Triazoles | >19 |
| 539 | 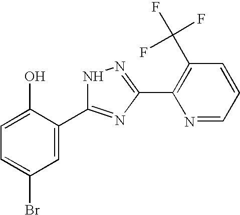 | Triazoles | 0.35 |
| 540 | 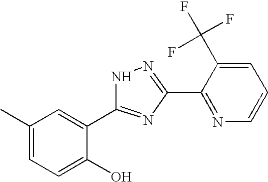 | Triazoles | >19 |
| 541 | 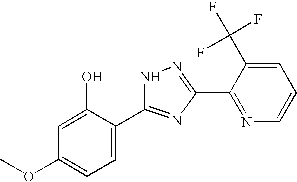 | Triazoles | >19 |
| 542 | 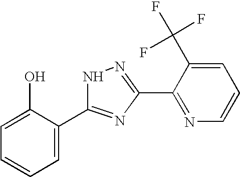 | Triazoles | >19 |
| 543 | 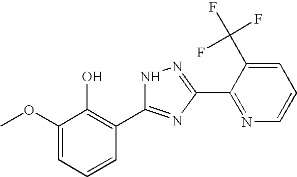 | Triazoles | >19 |
| 544 | 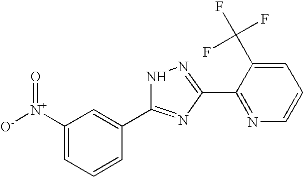 | Triazoles | >19 |
| 545 | 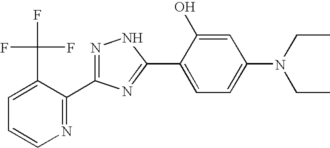 | Triazoles | >19 |
| 546 | 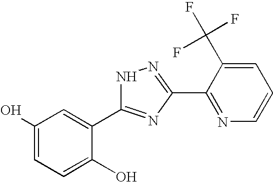 | Triazoles | >19 |
| 547 | 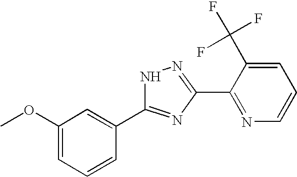 | Triazoles | >19 |
| 548 | 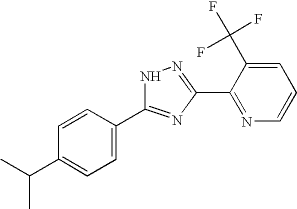 | Triazoles | >19 |
| 549 | 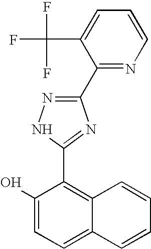 | Triazoles | >19 |
| 550 | 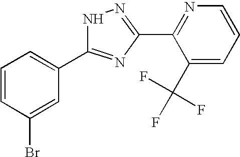 | Triazoles | >19 |
| 551 | 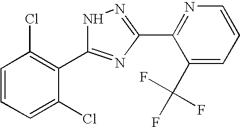 | Triazoles | >19 |
| 552 | 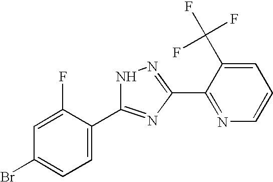 | Triazoles | >19 |
| 553 | 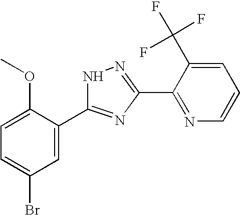 | Triazoles | >19 |
| 554 | 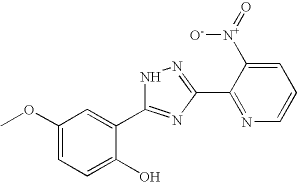 | Triazoles | >19 |
| 555 | 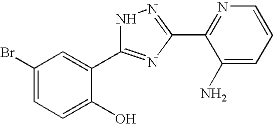 | Triazoles | >3.2 |
| 556 | 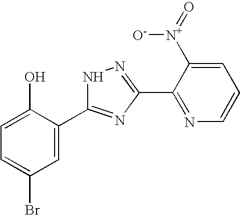 | Triazoles | >3.2 |
| 557 | 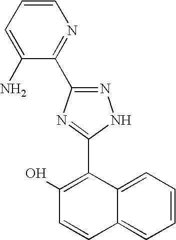 | Triazoles | >3.2 |
| 558 | 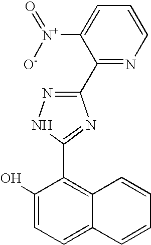 | Triazoles | >3.2 |
| 559 | 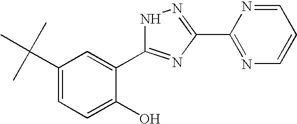 | Triazoles | >32 |
| 560 | 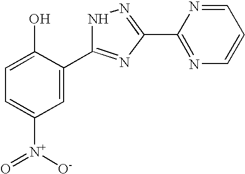 | Triazoles | >32 |
| 561 | 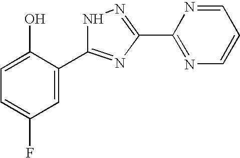 | Triazoles | >32 |
| 562 | 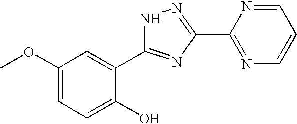 | Triazoles | >32 |
| 563 | 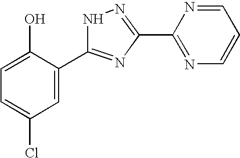 | Triazoles | 6.4 |
| 564 | 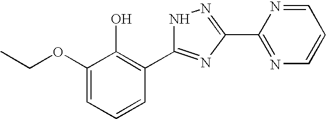 | Triazoles | >32 |
| 565 | 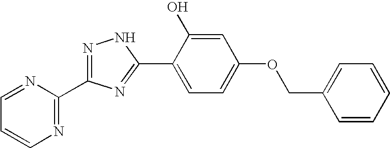 | Triazoles | 26 |
| 566 | 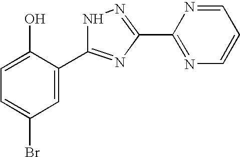 | Triazoles | 5.9 |
| 567 | 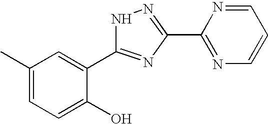 | Triazoles | >32 |
| 568 | 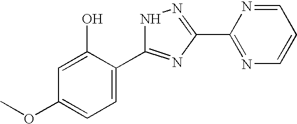 | Triazoles | >32 |
| 569 | 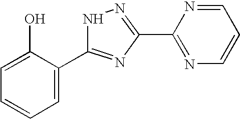 | Triazoles | >32 |
| 570 | 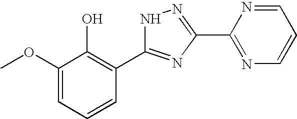 | Triazoles | >32 |
| 571 | 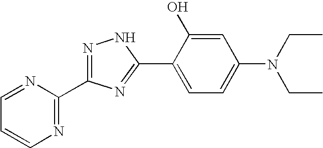 | Triazoles | 18 |
| 572 | 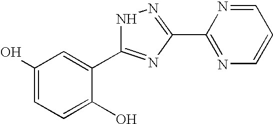 | Triazoles | >32 |
| 573 | 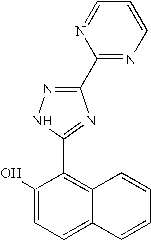 | Triazoles | 3.5 |
| 574 | 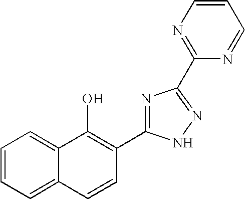 | Triazoles | >32 |
| 575 | 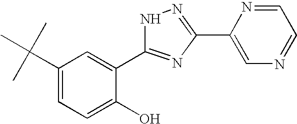 | Triazoles | 9 |
| 576 | 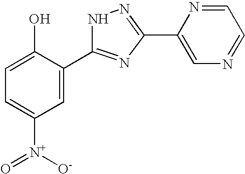 | Triazoles | 16 |
| 577 | 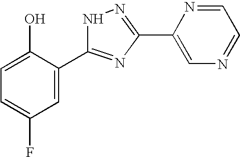 | Triazoles | 18 |
| 578 | 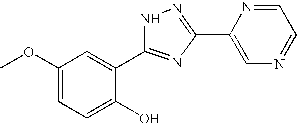 | Triazoles | >32 |
| 579 | 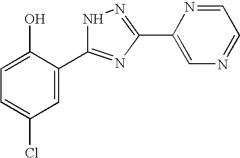 | Triazoles | 5.8 |
| 580 | 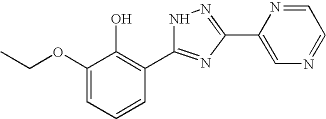 | Triazoles | >32 |
| 581 | 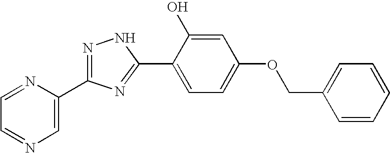 | Triazoles | >32 |
| 582 | 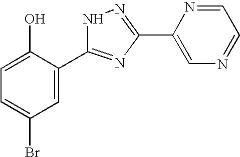 | Triazoles | 18 |
| 583 | 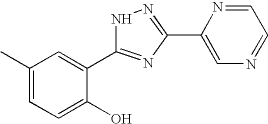 | Triazoles | >32 |
| 584 | 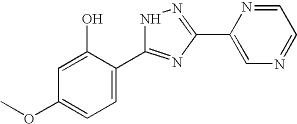 | Triazoles | >32 |
| 585 | 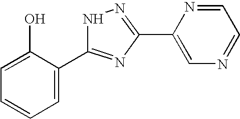 | Triazoles | >32 |
| 586 | 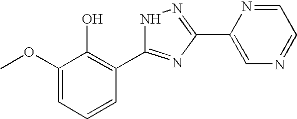 | Triazoles | >32 |
| 587 | 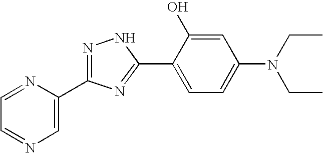 | Triazoles | 23 |
| 588 | 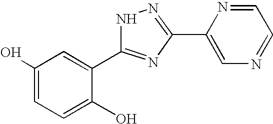 | Triazoles | >32 |
| 589 | 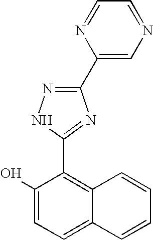 | Triazoles | 7 |
| 590 | 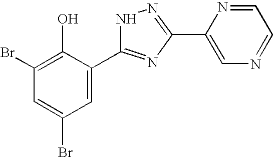 | Triazoles | 14 |
| 591 | 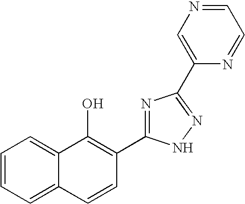 | Triazoles | 16 |
| 592 | 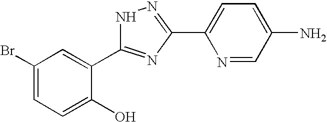 | Triazoles | >32 |
| 593 | 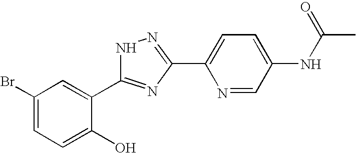 | Triazoles | 48.7 |
| 594 | 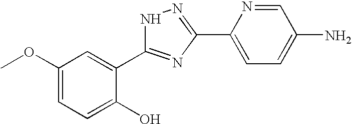 | Triazoles | >19 |
| 595 | 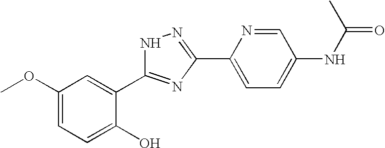 | Triazoles | 41.8 |
| 596 | 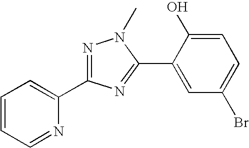 | Triazoles | 15 |
| 597 | 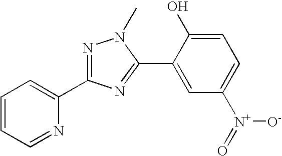 | Triazoles | 11 |
| 598 | 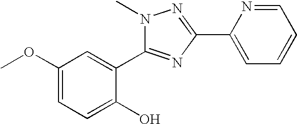 | Triazoles | >32 |
| 599 | 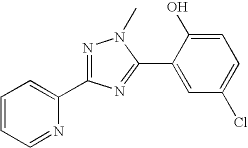 | Triazoles | 12 |
| 600 | 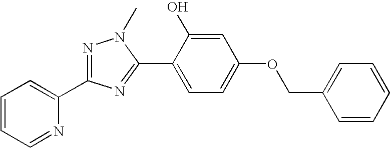 | Triazoles | 5 |
| 601 | 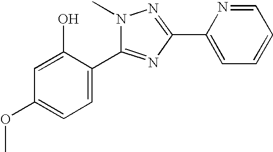 | Triazoles | >32 |
| 602 | 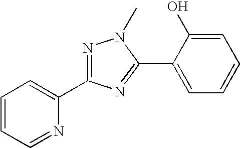 | Triazoles | >32 |
| 603 | 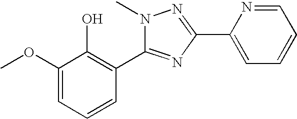 | Triazoles | >32 |
| 604 | 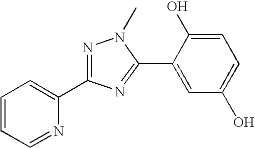 | Triazoles | 37.6 |
| 605 | 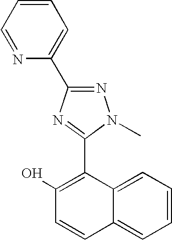 | Triazoles | 9 |
| 606 | 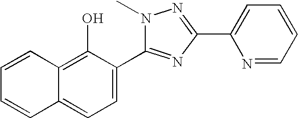 | Triazoles | 2.4 |
| 607 | 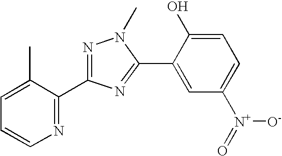 | Triazoles | 2.8 |
| 608 | 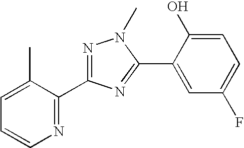 | Triazoles | 8.1 |
| 609 | 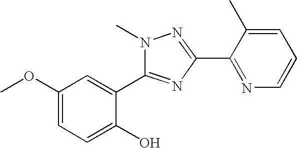 | Triazoles | 17 |
| 610 | 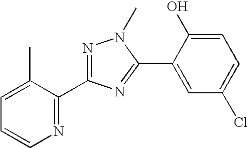 | Triazoles | 2.5 |
| 611 | 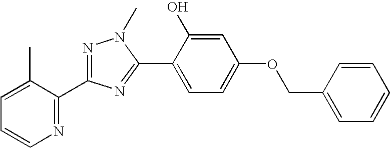 | Triazoles | >3.2 |
| 612 | 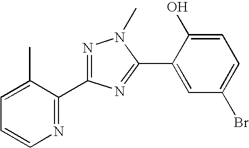 | Triazoles | 2.4 |
| 613 | 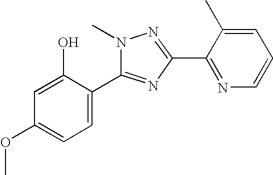 | Triazoles | 6.4 |
| 614 | 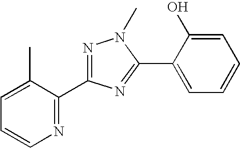 | Triazoles | 6.9 |
| 615 | 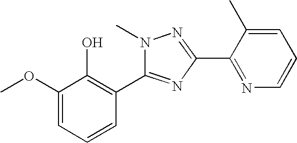 | Triazoles | 18 |
| 616 | 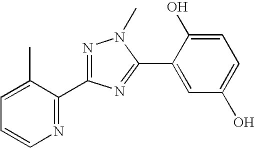 | Triazoles | 35.8 |
| 617 | 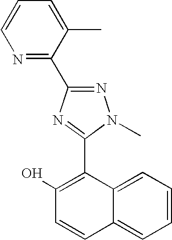 | Triazoles | >19 |
| 618 | 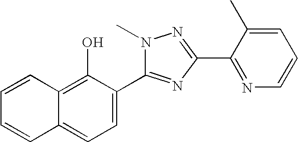 | Triazoles | 1.5 |
| 619 | 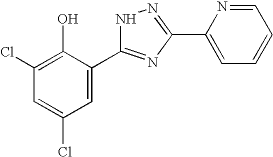 | Triazoles | 0.3 |
| 620 | 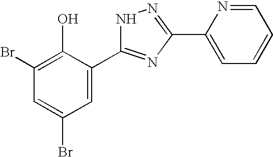 | Triazoles | 0.6 |
| 621 | 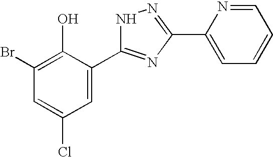 | Triazoles | 0.5 |
| 622 | 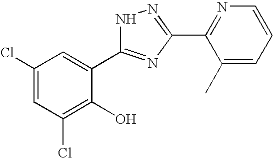 | Triazoles | 1.4 |
| 623 | 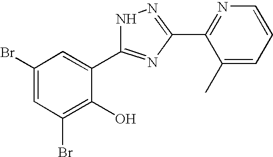 | Triazoles | 1.5 |
| 624 | 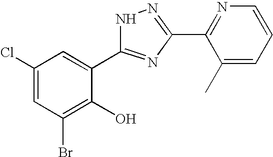 | Triazoles | 2.2 |
| 625 | 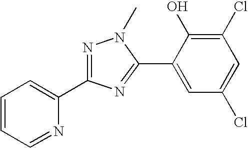 | Triazoles | 3.2 |
| 626 | 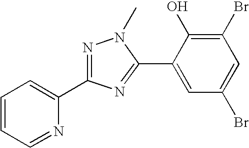 | Triazoles | 4.8 |
| 627 | 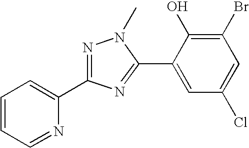 | Triazoles | 2.5 |
| 628 | 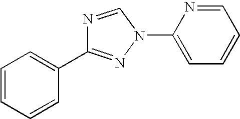 | Triazoles | >32 |
| 629 | 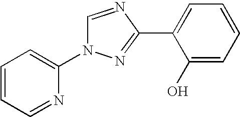 | Triazoles | 16 |
| 630 | 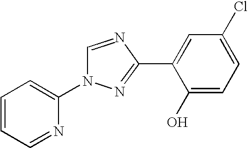 | Triazoles | 11 |
| 631 | 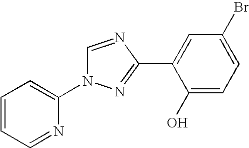 | Triazoles | 1.4 |
| 632 | 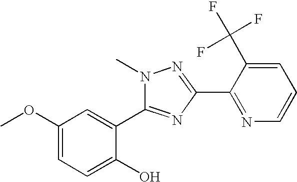 | Triazoles | 31 |
| 633 | 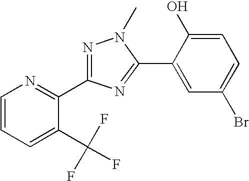 | Triazoles | 3.6 |
| 634 | 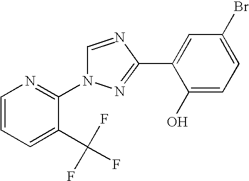 | Triazoles | 3.8 |
| 635 | 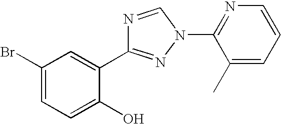 | Triazoles | 6.2 |
| 636 | 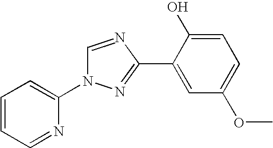 | Triazoles | 15 |
| 637 | 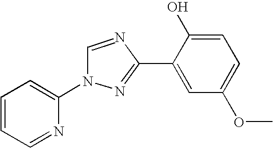 | Triazoles | 12 |
| 638 | 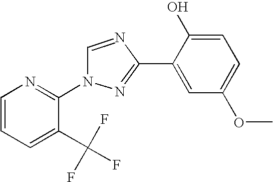 | Triazoles | 11 |
| 639 | 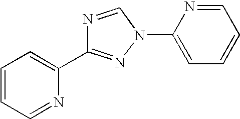 | Triazoles | >32 |
| 640 | 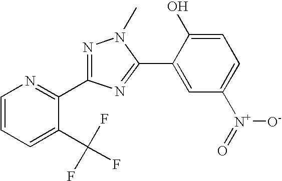 | Triazoles | >32 |

1. The compound according to Formula I: 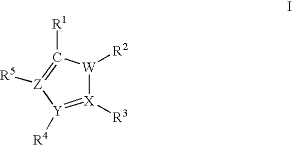
wherein:
X, Y, and Z are independently selected from C and N atoms;
W is selected from N, O, and S atoms;
R¹, R³, R⁴, and R⁵ are independently selected from hydrogen, halogen, nitro, or an unsubstituted or substituted alkyl, alkoxy, aryl, heteroaryl, Ring A, and Ring B, or R⁴ and R⁵ combine to form, together with Y and Z, a substituted five- or six-membered heterocycloalkyl;
R² is selected from hydrogen or an unsubstituted or substituted alkyl, Ring A, and Ring B when W is N;
wherein at least two of W, X, Y, and Z are heteroatoms;
wherein one of the R¹-R⁵ groups is Ring A and one of the R¹-R⁵ groups is Ring B, and the remaining R¹-R⁵ groups are selected from hydrogen, halogen, nitro, or a unsubstituted or substituted alkyl and alkoxy; and
wherein Ring A and Ring B are independently selected from an unsubstituted or substituted aryl, alkylaryl, heterocycloalkyl, heteroaryl or alkylheteroaryl.

2. A compound of claim 1 selected from the group consisting of 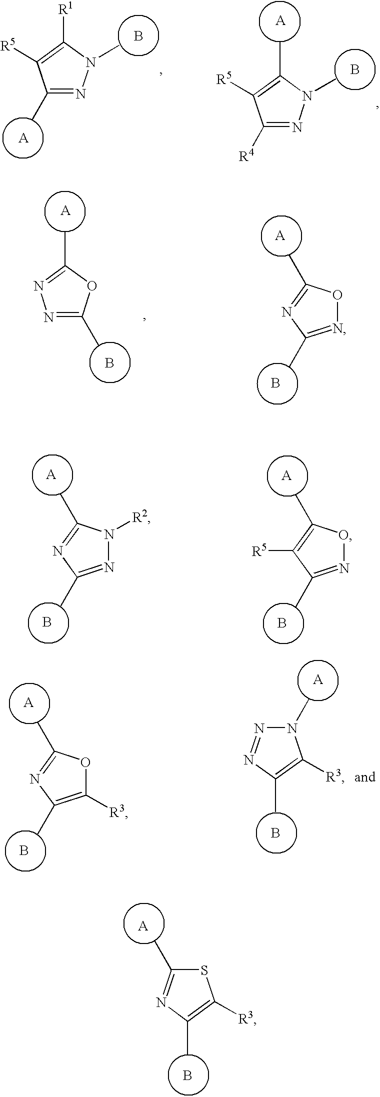
wherein Ring A is an unsubstituted or substituted aryl or heteroaryl selected from the group consisting of 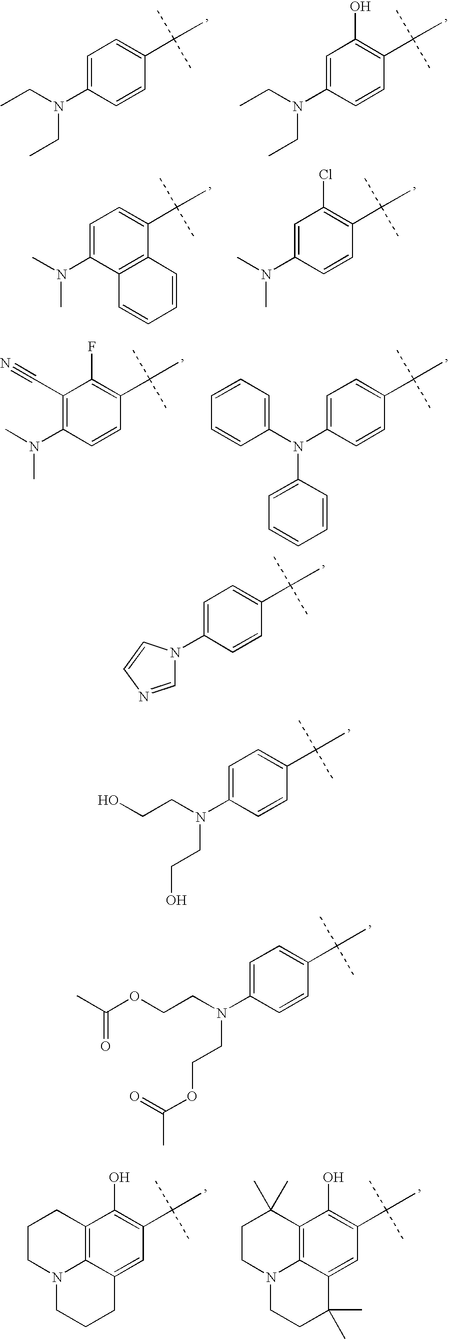 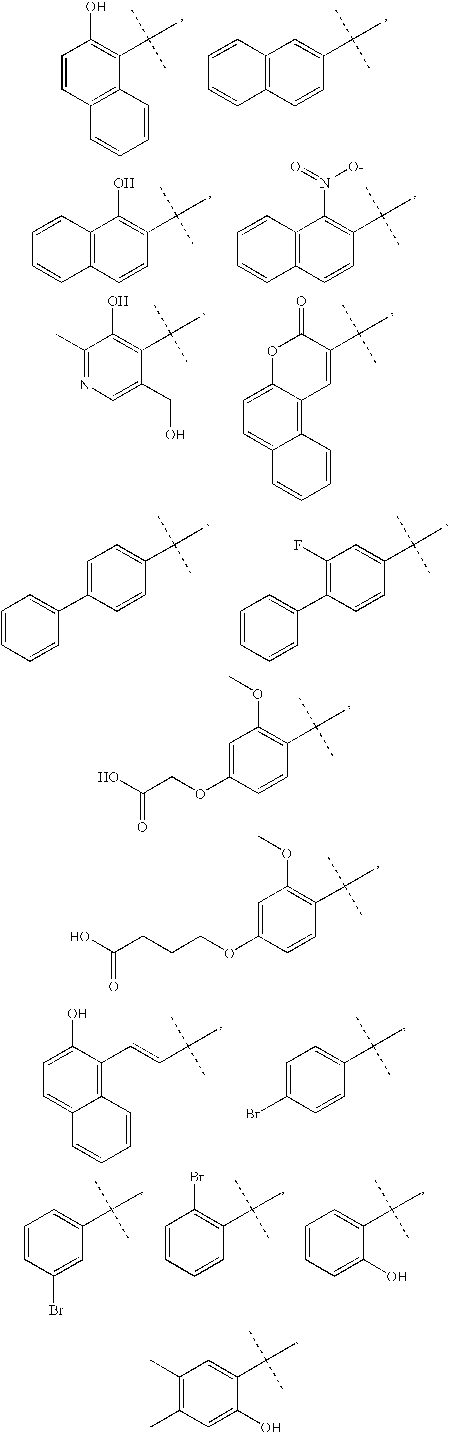 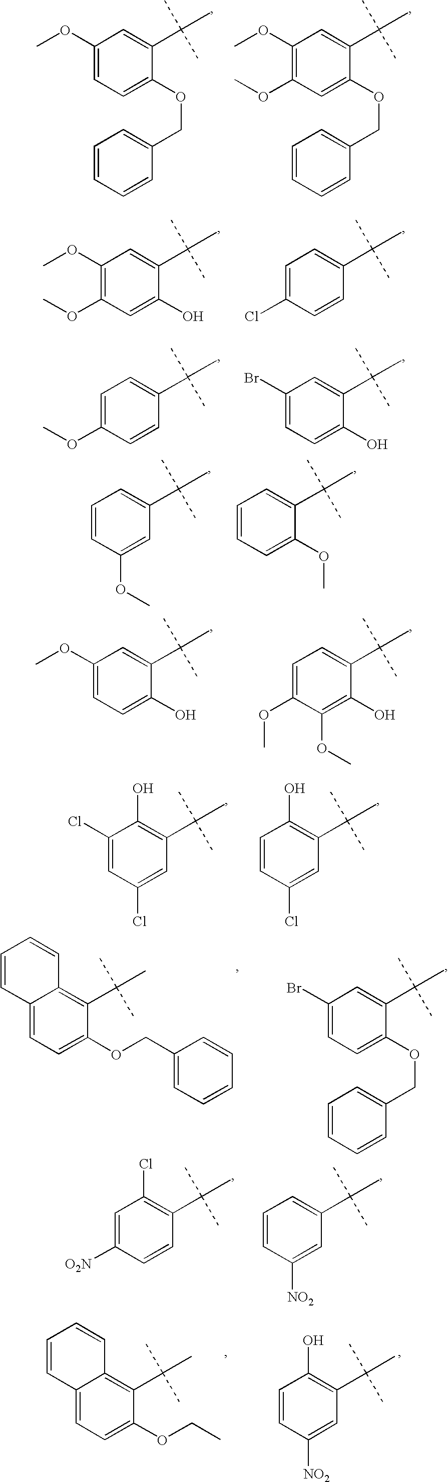 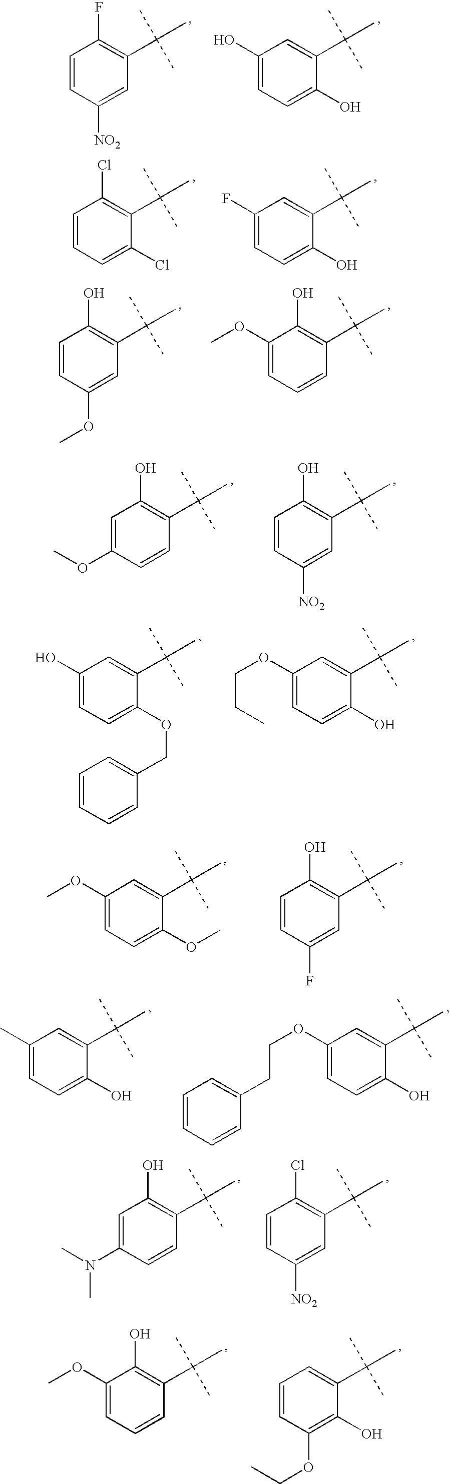 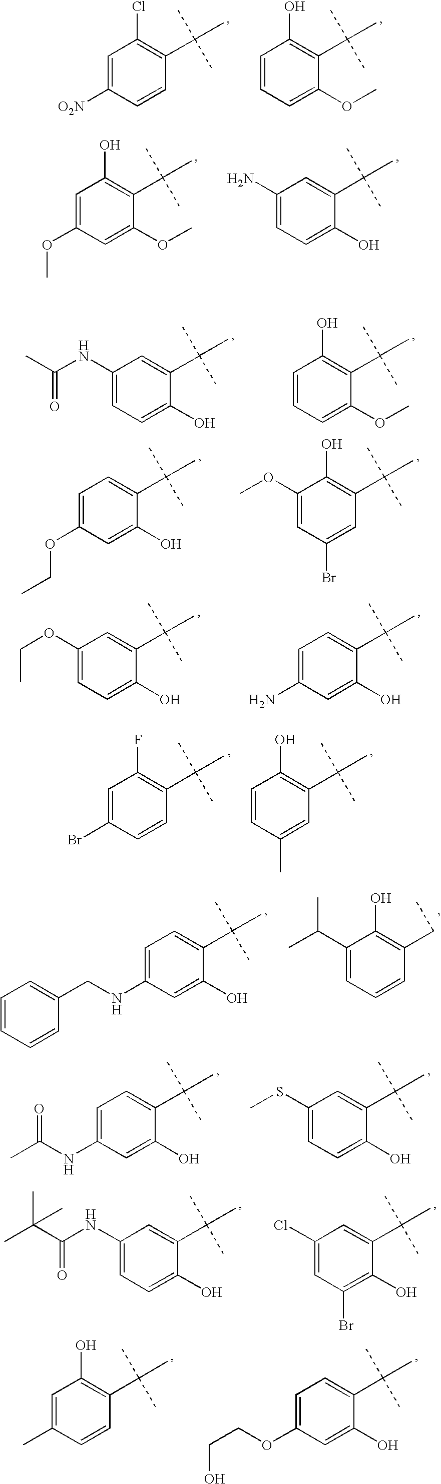 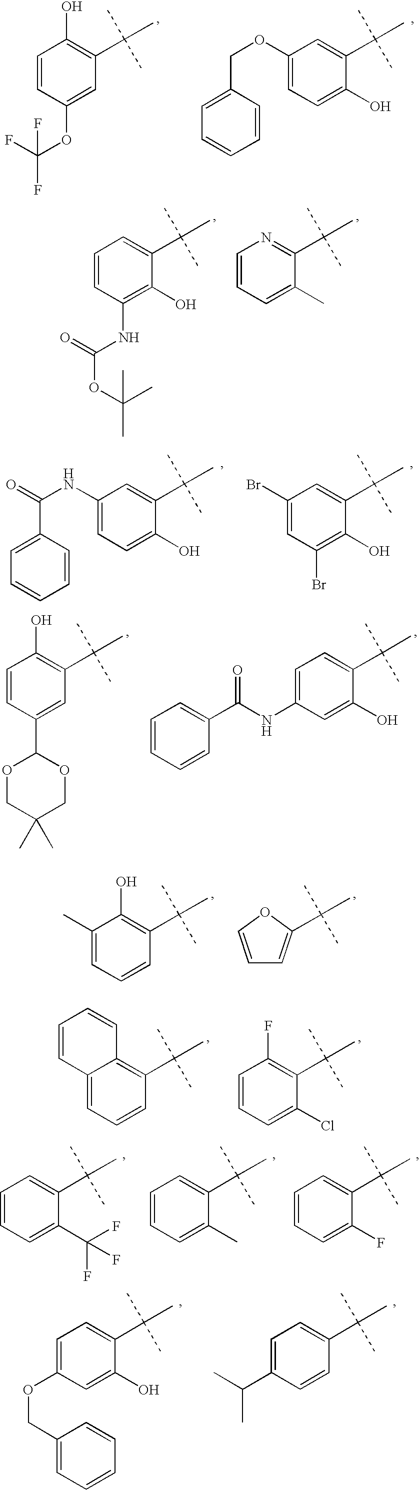 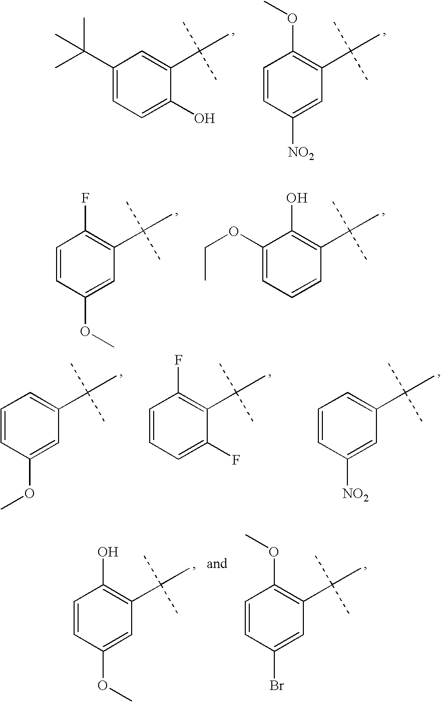
and Ring B is selected from the group consisting of 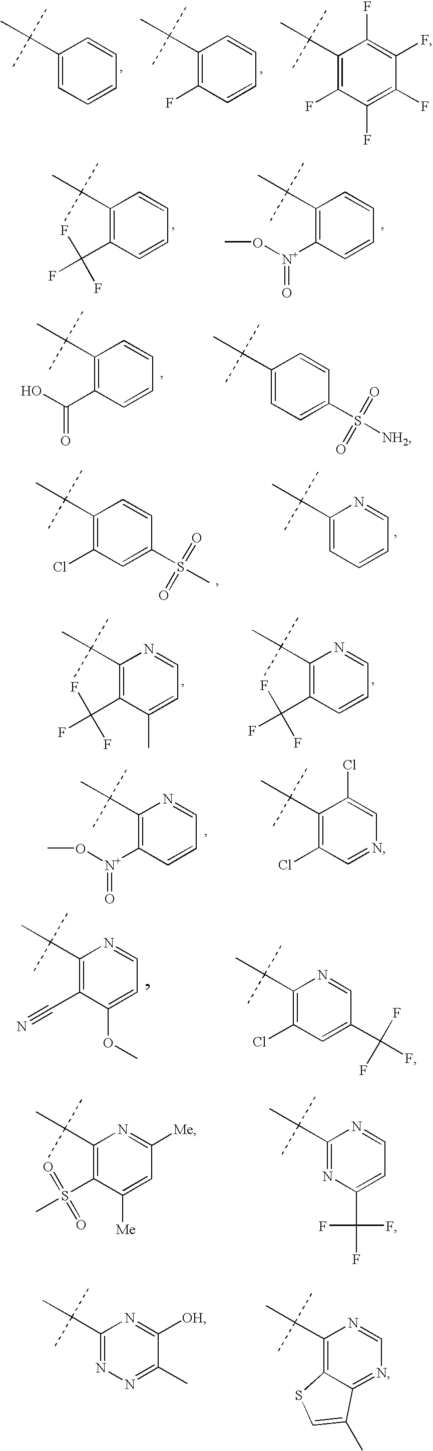 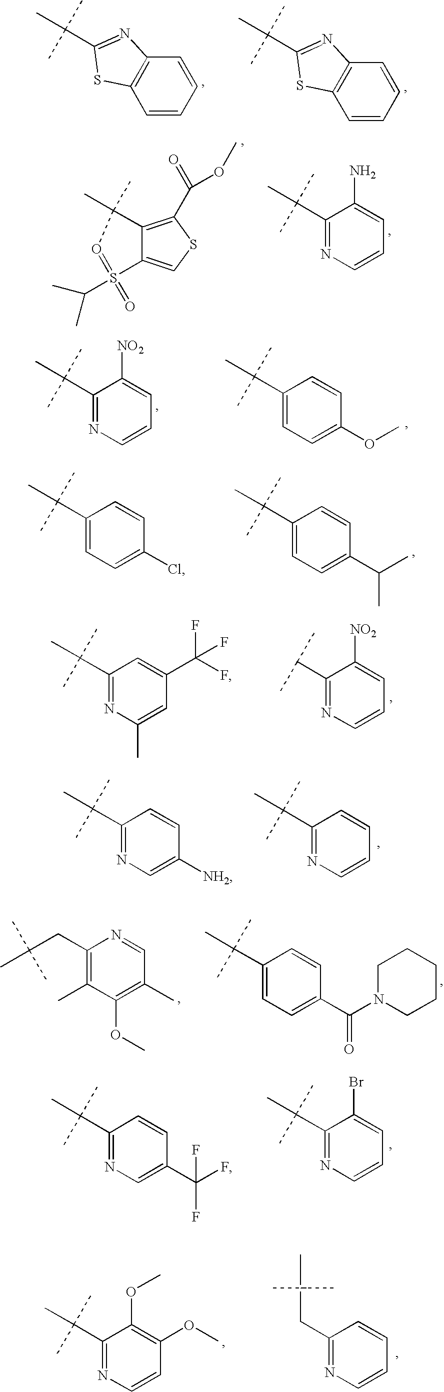 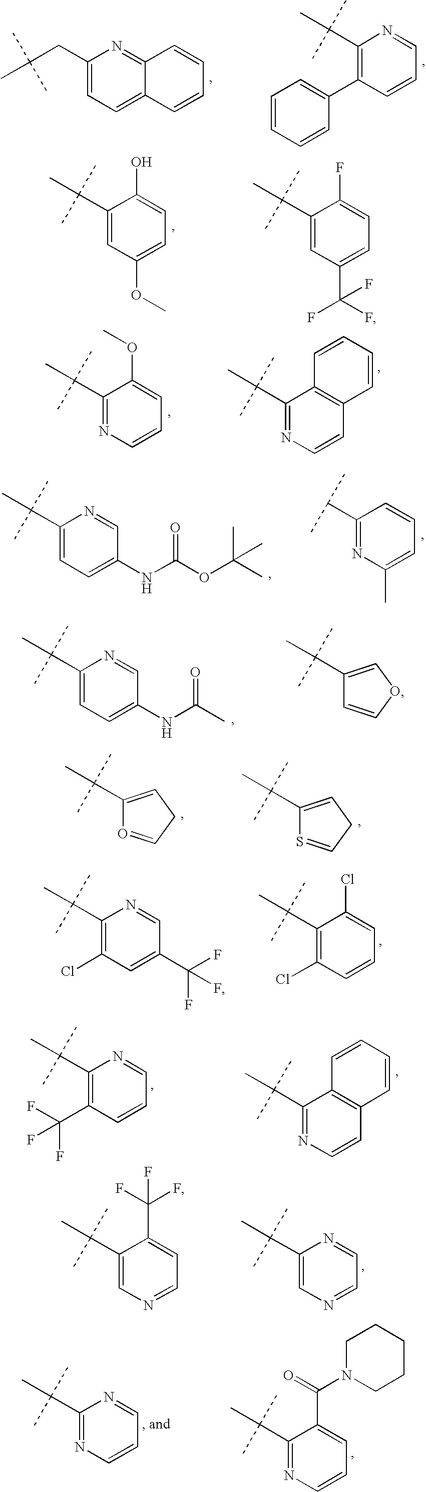
and R¹, R³, R⁴, and R⁵ other than Ring A and Ring B are selected from hydrogen, halogen, nitro, or an unsubstituted or substituted alkyl, or alkoxy, or R⁴ and R⁵ combine to form, together with Y and Z, a substituted five- or six-member heterocycolalkyl.

3. A compound of claim 1 selected from the group consisting of 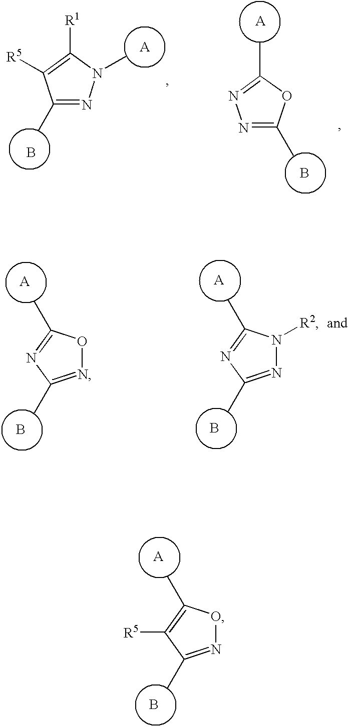
wherein Ring A is selected from the group consisting of 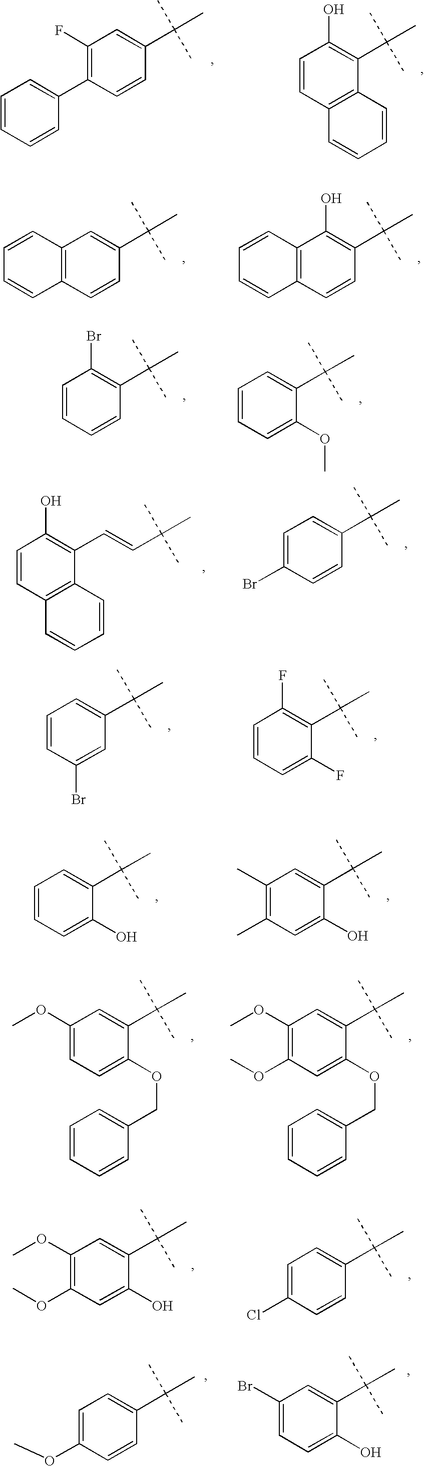 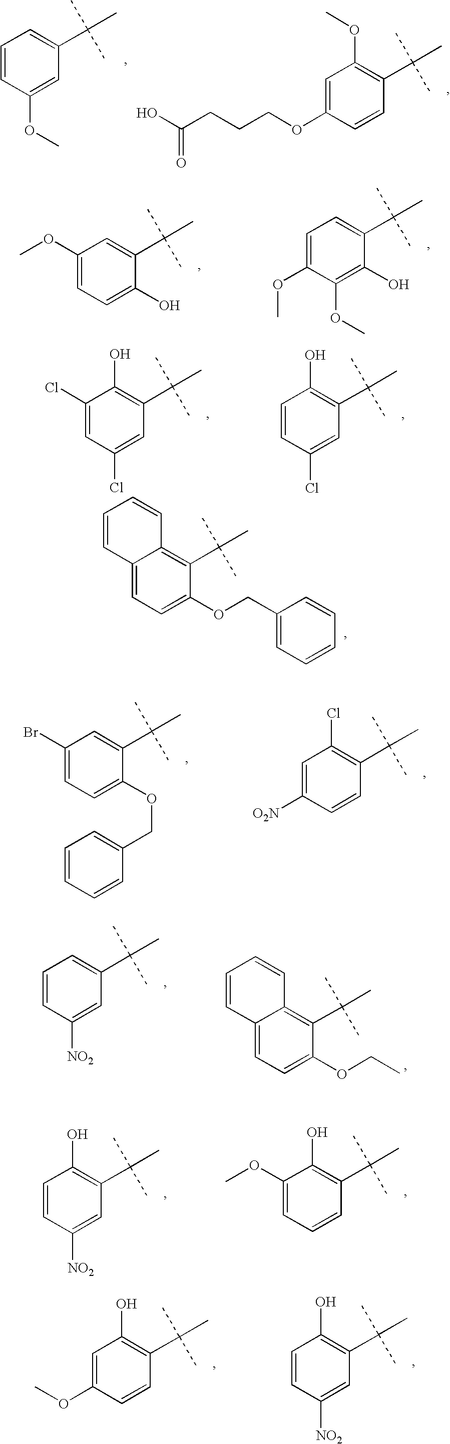 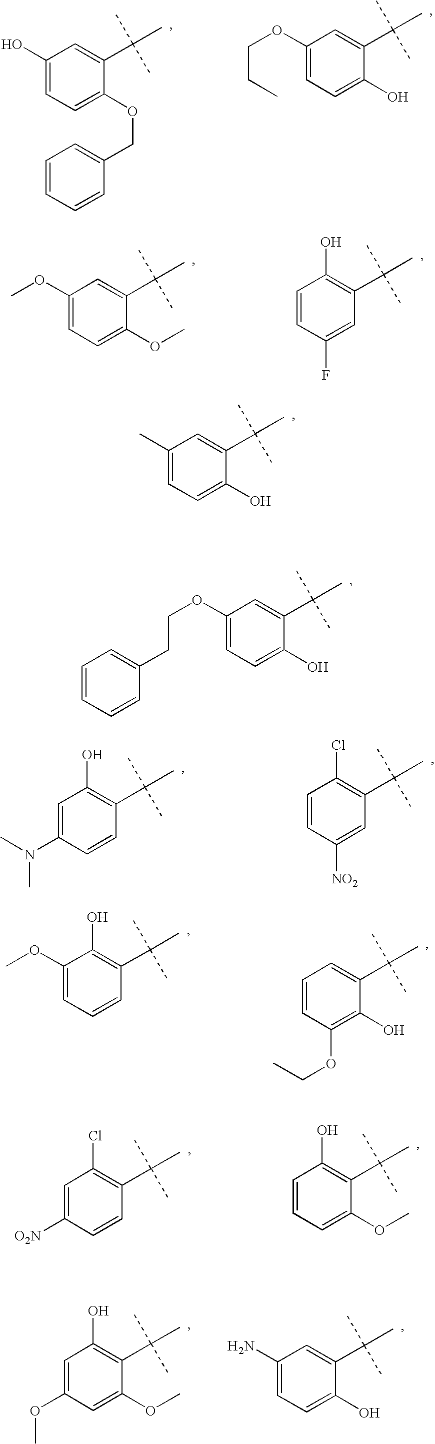 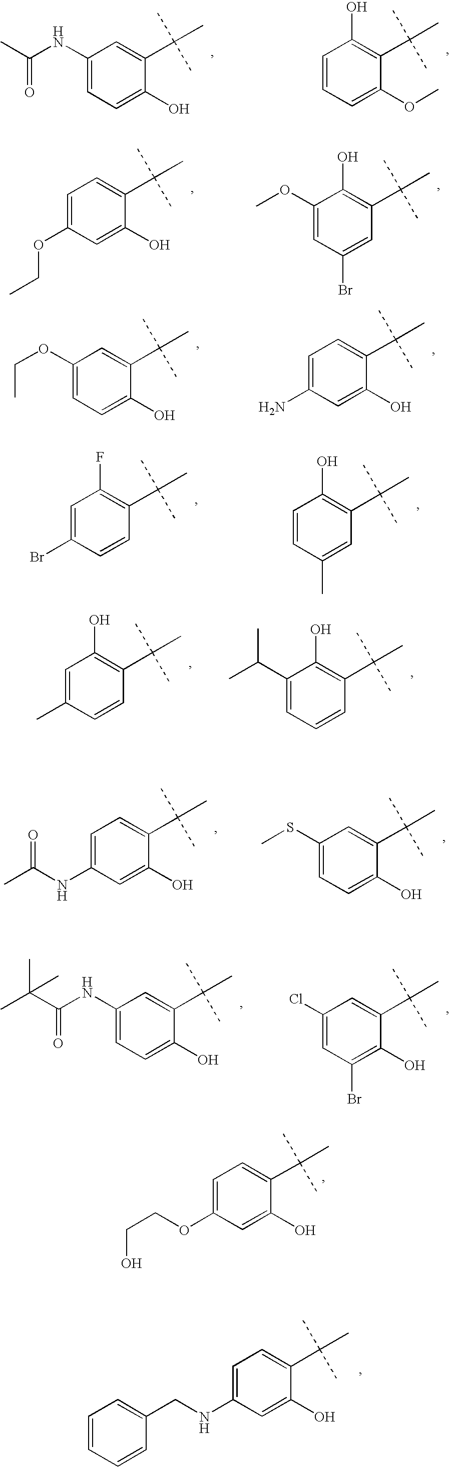 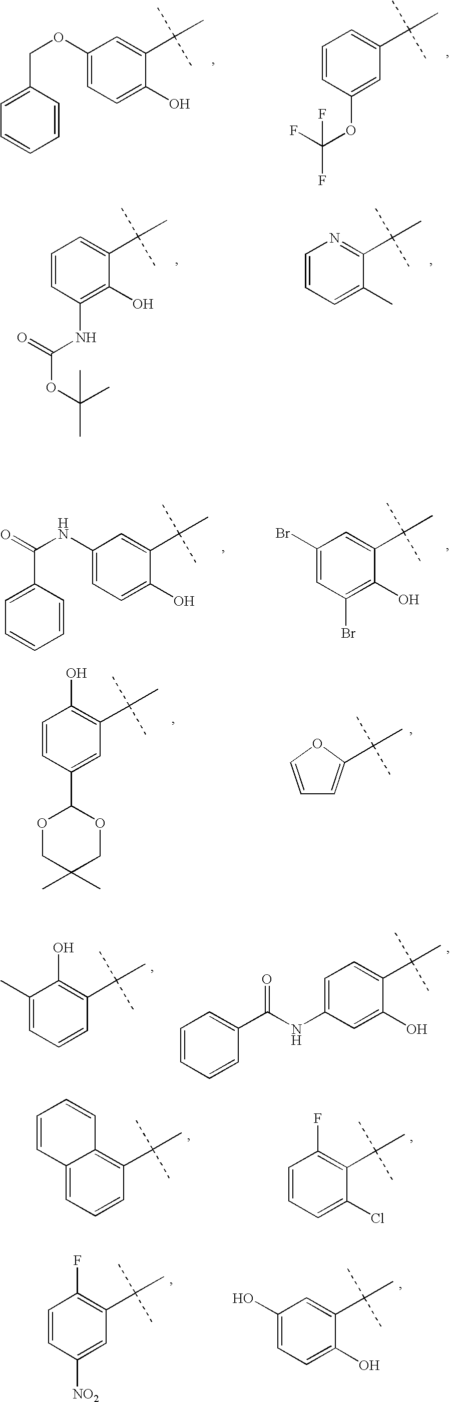 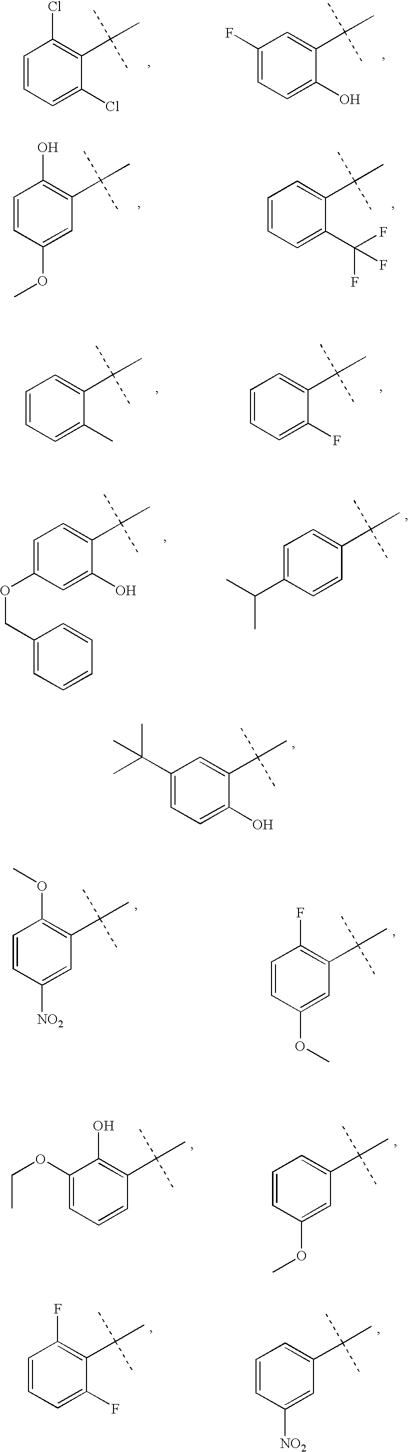 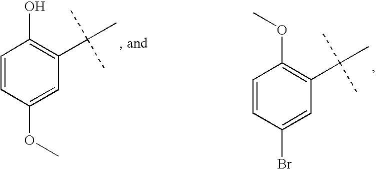
and wherein Ring B is selected from the group consisting of 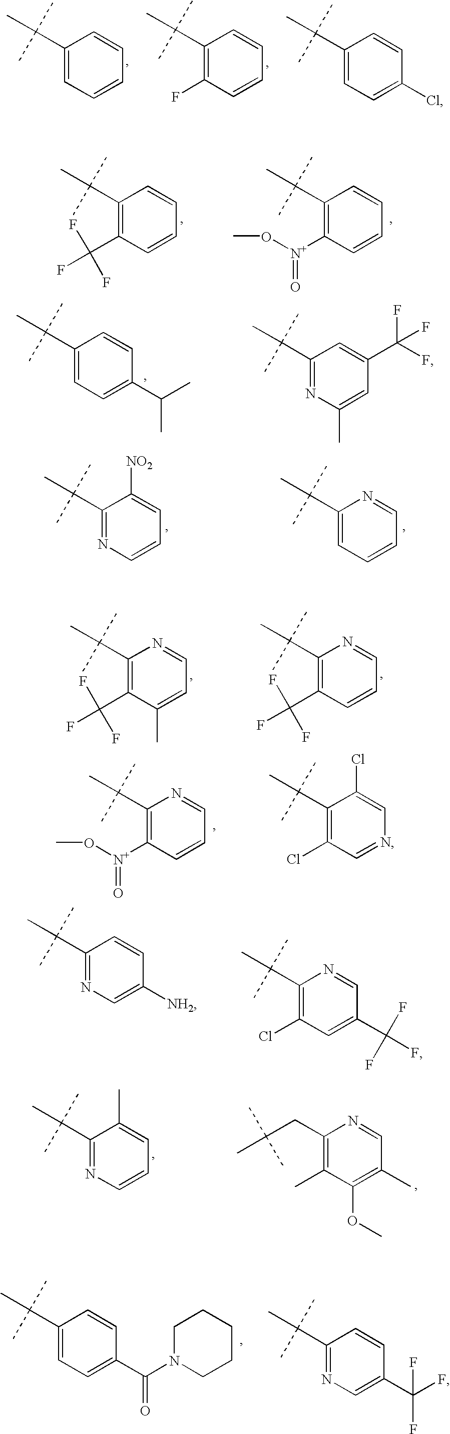 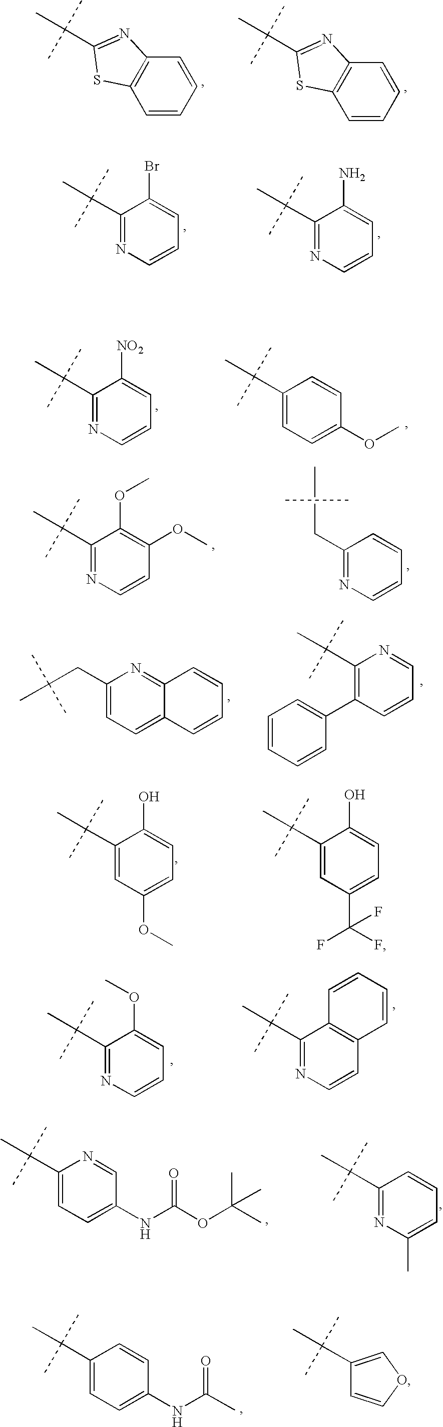 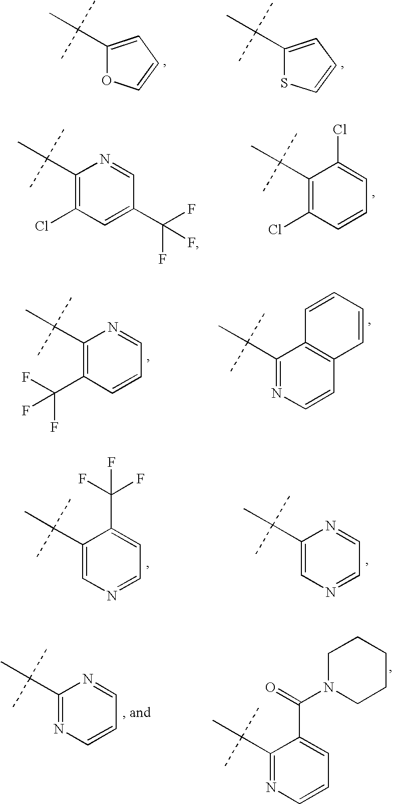
and R¹, R³, R⁴, and R⁵ other than Ring A and Ring B are selected from hydrogen or an unsubstituted or substituted alkyl, or R⁴ and R⁵ combine to form, together with Y and Z, a substituted five- or six-member heterocycolalkyl.

4. A compound selected from the group consisting of

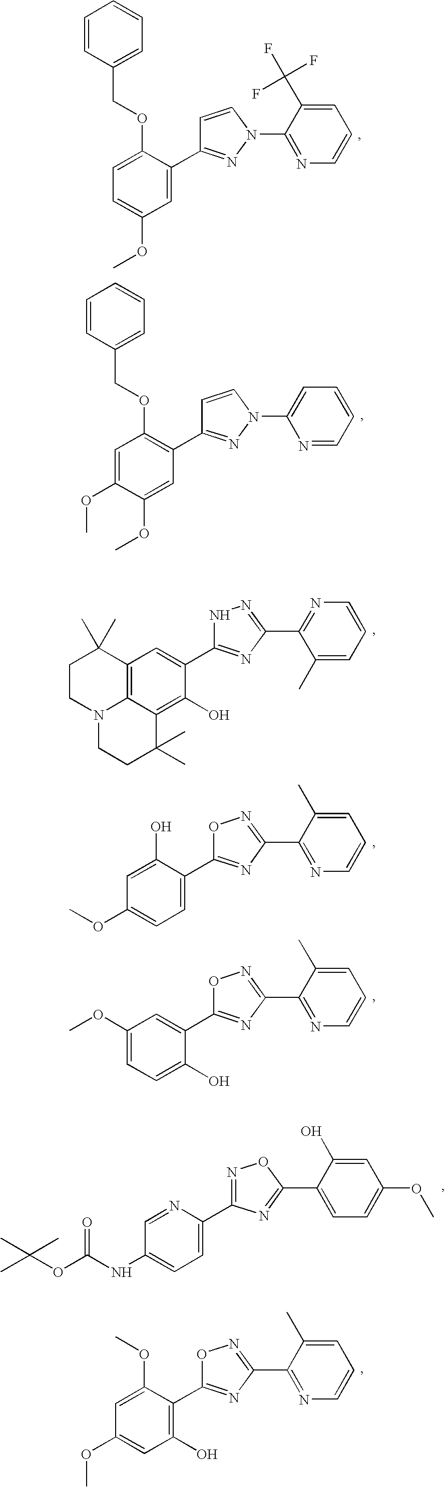

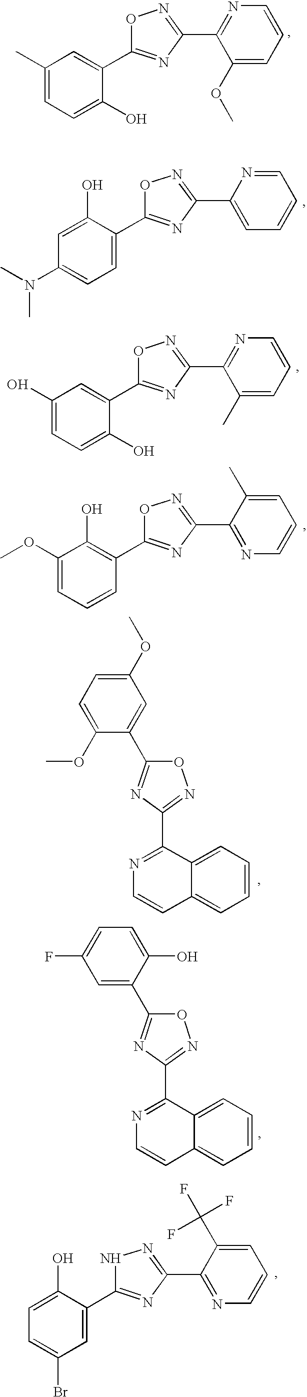

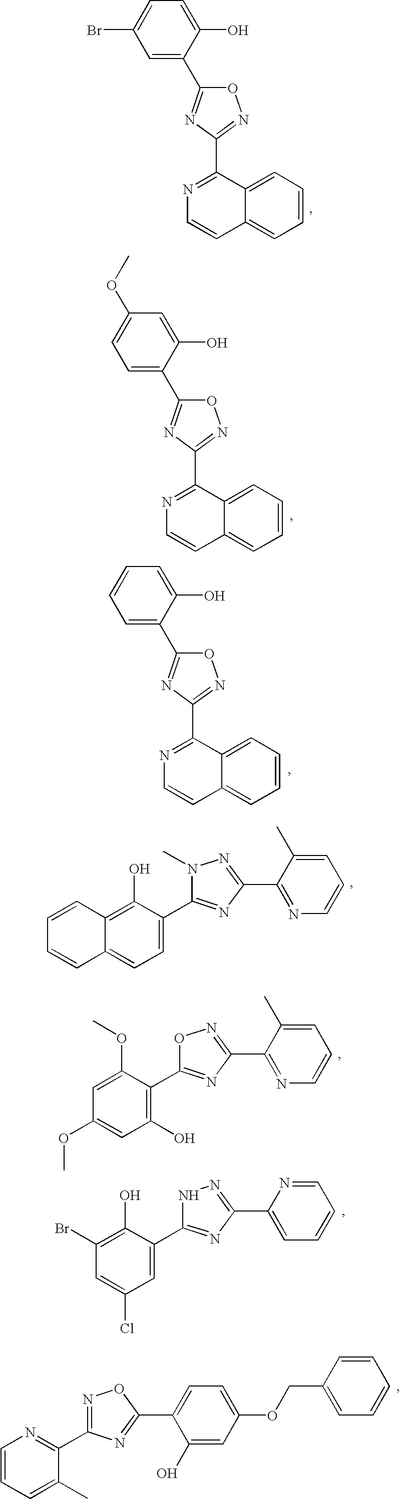

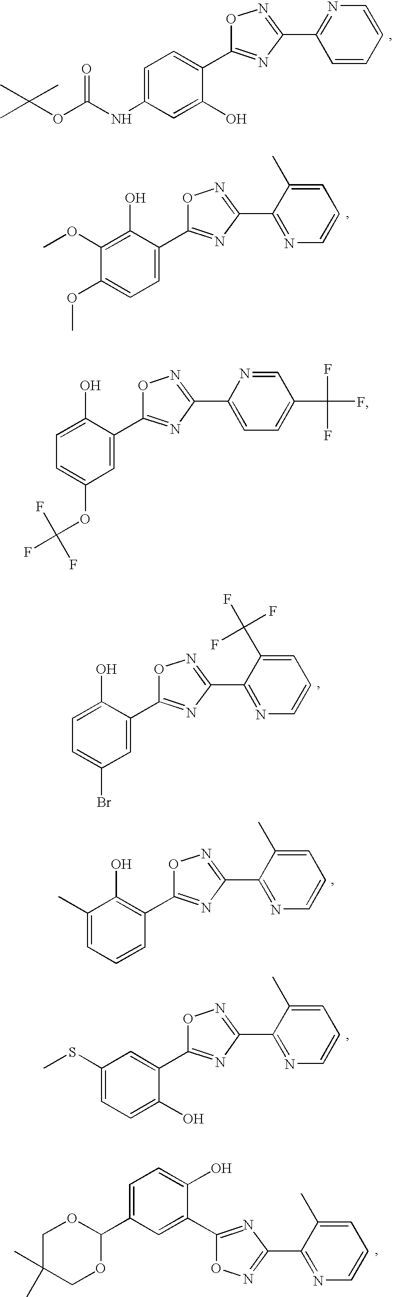

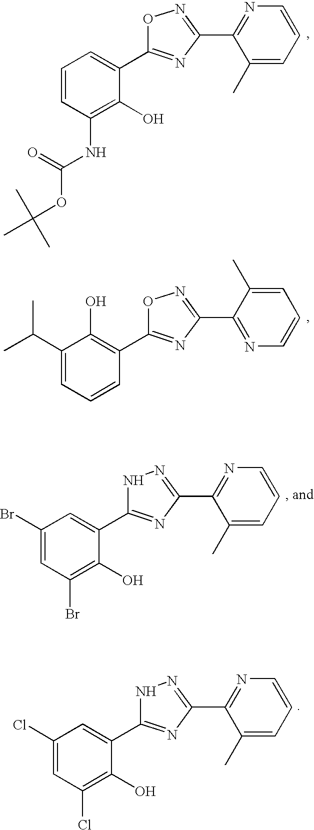

5. A compound selected from the group consisting of

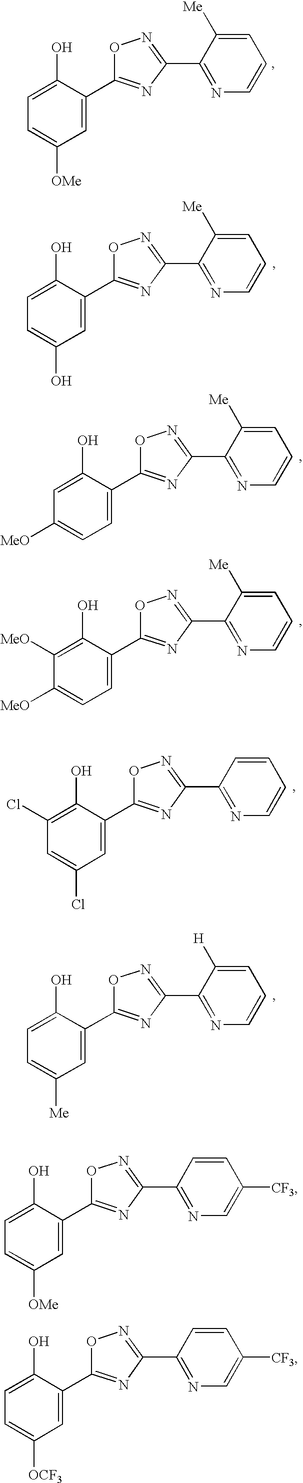

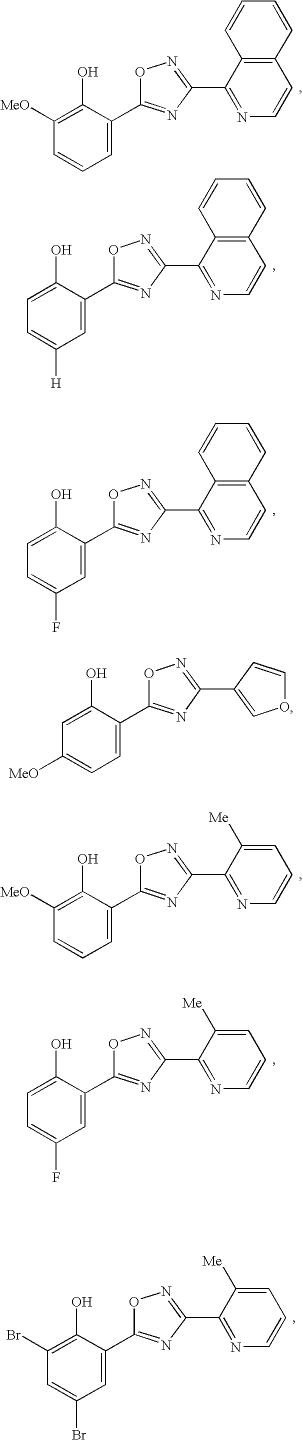

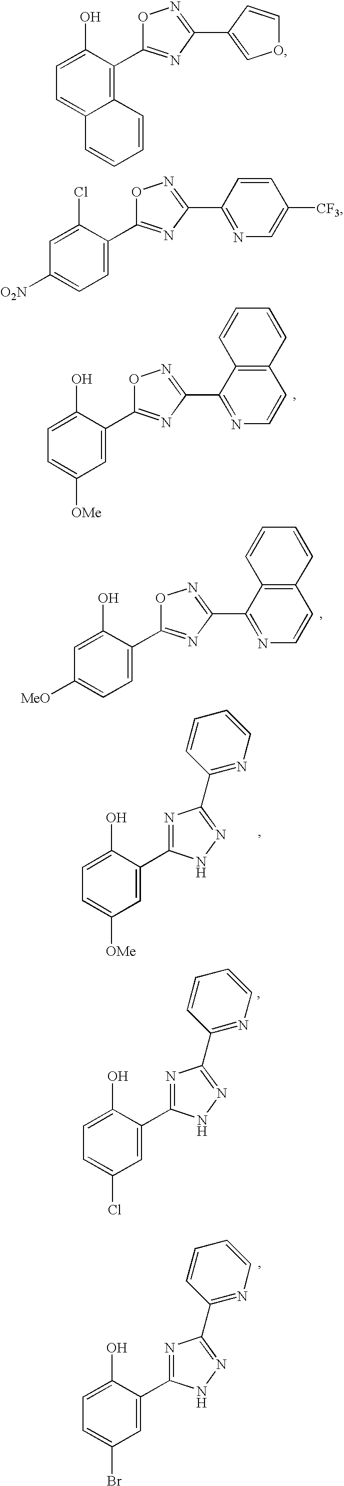

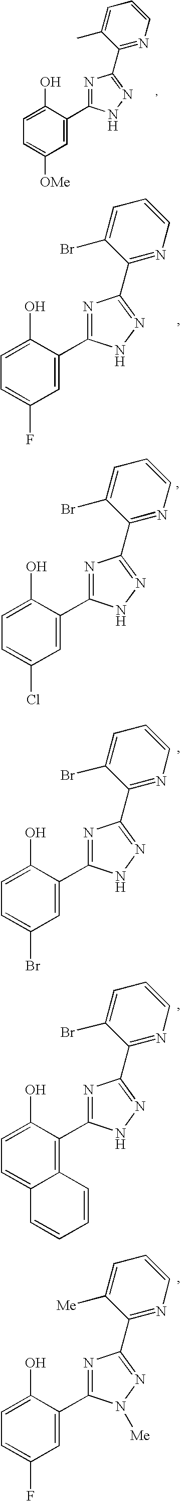

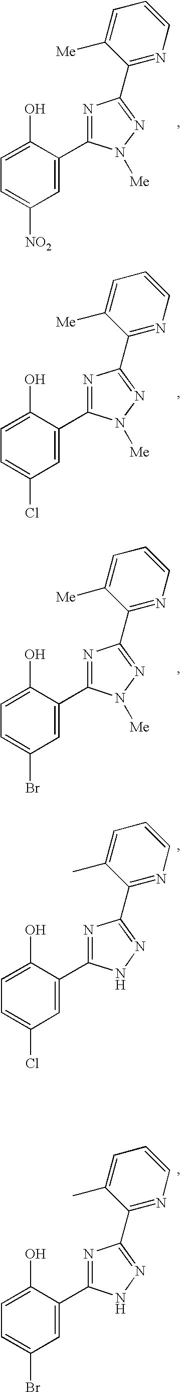

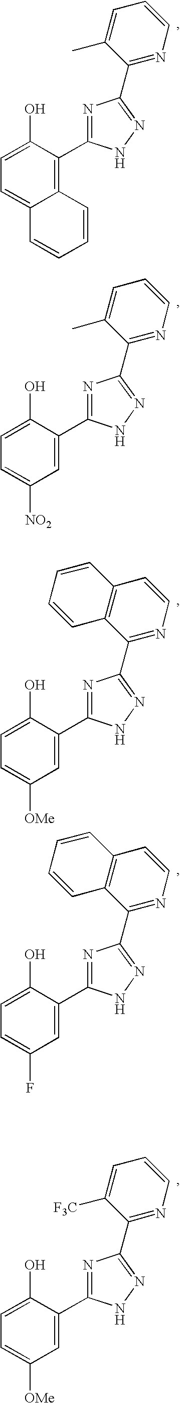

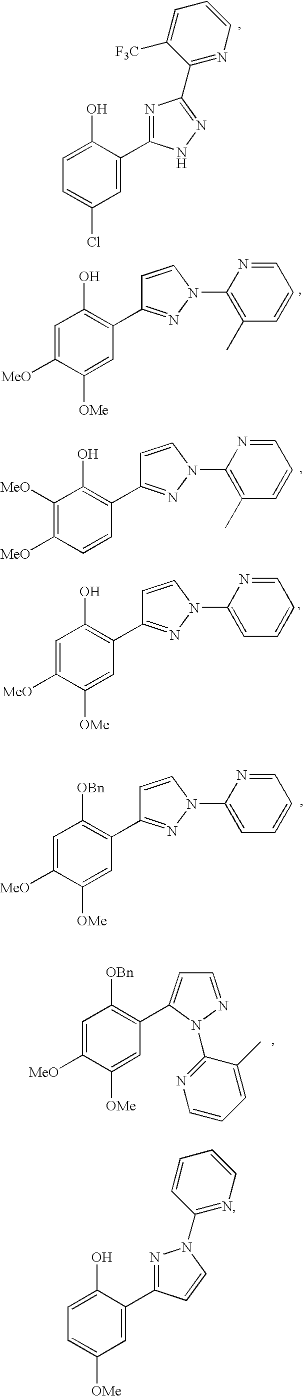

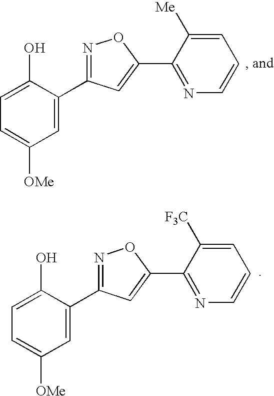

6. A pharmaceutical composition comprising a compound of Formula I and a pharmaceutically acceptable carrier.

7. A method of inhibiting Hepatitis C Virus replication in a cell comprising administering to the cell a composition comprising an effective amount of a compound of Formula I.

8. A method of inhibiting Hepatitis C Virus replication comprising administering to a subject in need thereof a composition comprising an effective amount of a compound of Formula I.
